SEQUENCE LISTING (320 sequences)

SEQ ID NO: 1 - DNA, 145 bp, Adeno-associated virus 2
  source 1..145: mol_type = genomic DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 2 - DNA, 113 bp, synthetic construct
  source 1..113: mol_type = other DNA
tccctctctgcgcgctcgctcgctcactgaggccgcccgggcaaagcccgggcgtcgggcgacctttggtcgcccggcctcagtgagcgagcgagcgcgcagagagggagtgg

SEQ ID NO: 3 - DNA, 138 bp, synthetic construct
  source 1..138: mol_type = other DNA
tccctctctgcgcgctcgctcgctcactgaggccgcccgggcaaagcccgggcgtcgggcgacctttggtcgcccggcctcagtgagcgagcgagcgcgcagagagggagtggactagctccatcactaggggttcct

SEQ ID NO: 4 - DNA, 20 bp, synthetic construct
  source 1..20: mol_type = other DNA
ctccatcactaggggttcct

SEQ ID NO: 5 - DNA, 145 bp, Adeno-associated virus 2
  source 1..145: mol_type = genomic DNA
aggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 6 - protein, 736 aa, Adeno-associated virus 1
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 7 - protein, 735 aa, Adeno-associated virus 2 [sequence omitted]

SEQ ID NO: 8 - protein, 736 aa, Adeno-associated virus 3
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKGAVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVRGVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTGTVNHQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 9 - protein, 736 aa, Adeno-associated virus 3
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 10 - protein, 734 aa, Adeno-associated virus 4
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQQRLQGDTSFGGNLGRAVFQAKKRVLEPLGLVEQAGETAPGKKRPLIESPQQPDSSTGIGKKGKQPAKKKLVFEDETGAGDGPPEGSTSGAMSDDSEMRAAAGGAAVEGGQGADGVGNASGDWHCDSTWSEGHVTTTSTRTWVLPTYNNHLYKRLGESLQSNTYNGFSTPWGYFDFNRFHCHFSPRDWQRLINNNWGMRPKAMRVKIFNIQVKEVTTSNGETTVANNLTSTVQIFADSSYELPYVMDAGQEGSLPPFPNDVFMVPQYGYCGLVTGNTSQQQTDRNAFYCLEYFPSQMLRTGNNFEITYSFEKVPFHSMYAHSQSLDRLMNPLIDQYLWGLQSTTTGTTLNAGTATTNFTKLRPTNFSNFKKNWLPGPSIKQQGFSKTANQNYKIPATGSDSLIKYETHSTLDGRWSALTPGPPMATAGPADSKFSNSQLIFAGPKQNGNTATVPGTLIFTSEEELAATNATDTDMWGNLPGGDQSNSNLPTVDRLTALGAVPGMVWQNRDIYYQGPIWAKIPHTDGHFHPSPLIGGFGLKHPPPQIFIKNTPVPANPATTFSSTPVNSFITQYSTGQVSVQIDWEIQKERSKRWNPEVQFTSNYGQQNSLLWAPDAAGKYTEPRAIGTRYLTHHL

SEQ ID NO: 11 - protein, 724 aa, Adeno-associated virus 5
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 12 - protein, 736 aa, Adeno-associated virus 6
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 13 - protein, 737 aa, Adeno-associated virus 7
MAADGYLPDWLEDNLSEGIREWWDLKPGAPKPKANQQKQDNGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPAKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 14 - protein, 738 aa, Adeno-associated virus 8
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 15 - protein, 736 aa, Adeno-associated virus 9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 16 - protein, 738 aa, Adeno-associated virus 10
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEAAKTAPGKKRPVEPSPQRSPDSSTGIGKKGQQPAKKRLNFGQTGESESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRDSLVNPGVAMATHKDDEERFFPSSGVLMFGKQGAGRDNVDYSSVMLTSEEEIKTTNPVATEQYGVVADNLQQANTGPIVGNVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 17 - protein, 733 aa, Adeno-associated virus 11
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPLESPQEPDSSSGIGKKGKQPARKRLNFEEDTGAGDGPPEGSDTSAMSSDIEMRAAPGGNAVDAGQGSDGVGNASGDWHCDSTWSEGKVTTTSTRTWVLPTYNNHLYLRLGTTSSSNTYNGFSTPWGYFDFNRFHCHFSPRDWQRLINNNWGLRPKAMRVKIFNIQVKEVTTSNGETTVANNLTSTVQIFADSSYELPYVMDAGQEGSLPPFPNDVFMVPQYGYCGIVTGENQNQTDRNAFYCLEYFPSQMLRTGNNFEMAYNFEKVPFHSMYAHSQSLDRLMNPLLDQYLWHLQSTTSGETLNQGNAATTFGKIRSGDFAFYRKNWLPGPCVKQQRFSKTASQNYKIPASGGNALLKYDTHYTLNNRWSNIAPGPPMATAGPSDGDFSNAQLIFPGPSVTGNTTTSANNLLFTSEEEIAATNPRDTDMFGQIADNNQNATTAPITGNVTAMGVLPGMVWQNRDIYYQGPIWAKIPHADGHFHPSPLIGGFGLKHPPPQIFIKNTPVPANPATTFTAARVDSFITQYSTGQVAVQIEWEIEKERSKRWNPEVQFTSNYGNQSSMLWAPDTTGKYTEPRVIGSRYLTNHL

SEQ ID NO: 18 - protein, 742 aa, Adeno-associated virus 12
MAADGYLPDWLEDNLSEGIREWWALKPGAPQPKANQQHQDNGRGLVLPGYKYLGPFNGLDKGEPVNEADAAALEHDKAYDKQLEQGDNPYLKYNHADAEFQQRLATDTSFGGNLGRAVFQAKKRILEPLGLVEEGVKTAPGKKRPLEKTPNRPTNPDSGKAPAKKKQKDGEPADSARRTLDFEDSGAGDGPPEGSSSGEMSHDAEMRAAPGGNAVEAGQGADGVGNASGDWHCDSTWSEGRVTTTSTRTWVLPTYNNHLYLRIGTTANSNTYNGFSTPWGYFDFNRFHCHFSPRDWQRLINNNWGLRPKSMRVKIFNIQVKEVTTSNGETTVANNLTSTVQIFADSTYELPYVMDAGQEGSFPPFPNDVFMVPQYGYCGVVTGKNQNQTDRNAFYCLEYFPSQMLRTGNNFEVSYQFEKVPFHSMYAHSQSLDRMMNPLLDQYLWHLQSTTTGNSLNQGTATTTYGKITTGDFAYYRKNWLPGACIKQQKFSKNANQNYKIPASGGDALLKYDTHTTLNGRWSNMAPGPPMATAGAGDSDFSNSQLIFAGPNPSGNTTTSSNNLLFTSEEEIATTNPRDTDMFGQIADNNQNATTAPHIANLDAMGIVPGMVWQNRDIYYQGPIWAKVPHTDGHFHPSPLMGGFGLKHPPPQIFIKNTPVPANPNTTFSAARINSFLTQYSTGQVAVQIDWEIQKEHSKRWNPEVQFTSNYGTQNSMLWAPDNAGNYHELRAIGSRFLTHHL

SEQ ID NO: 19 - protein, 733 aa, Adeno-associated virus 13
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQERLQEDTSFGGNLGRAVFQAKKRILEPLGLVEEAAKTAPGKKRPVEQSPAEPDSSSGIGKSGQQPARKRLNFGQTGDTESVPDPQPLGQPPAAPSGVGSTTMASGGGAPMADNNEGADGVGNSSGNWHCDSQWLGDRVITTSTRTWALPTYNNHLYKQISSQSGATNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQTASGTQQSRLLFSQAGPTSMSLQAKNWLPGPCYRQQRLSKQANDNNNSNFPWTGATKYHLNGRDSLVNPGPAMASHKDDKEKFFPMHGTLIFGKEGTNANNADLENVMITDEEEIRTTNPVATEQYGTVSNNLQNSNAGPTTGTVNHQGALPGMVWQDRDVYLQGPIWAKIPHTDGHFHPSPLMGGFGLKHPPPQIMIKNTPVPANPPTNFSAAKFASFITQYSTGQVSVEIEWELQKENSKRWNPEIQYTSNYNKSVNVDFTVDTNGVYSEPRPIGTRYLTRNL

SEQ ID NO: 20 - protein, 737 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRLLEPLGLVEEAAKTAPGKKRPVEHSPVEPDSSSGTGKAGQQPARKRLNFGQTGDADSVPDPQPIGEPPAAPSGVGSLTMAAGGGAPMADNNEGADGVGNSSGNWHCDSTWMGDRVITTSTRTWALPTYNNHLYKQISNSTSGGSSNDNAYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTTNTQTLGFSQGGPNTMANQAKNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 21 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSDGTLAVPFKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 22 - protein, 736 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKKGQQPARKRLNFGQTGDSESVPDPQPLGEPPAAPSGVGSNTMAAGGGAPMADNNEGADGVGNASGNWHCDSTWLGDRVITTSTRTWALPTYNNHLYKQISSQSGGSTNDNTYFGYSTPWGYFDFNRFHCHFSPRDWQRLINNNWGFRPKKLNFKLFNIQVKEVTTNDGTTTIANNLTSTVQVFTDSEYQLPYVLGSAHQGCLPPFPADVFMIPQYGYLTLNNGSQAVGRSSFYCLEYFPSQMLRTGNNFQFSYTFEDVPFHSSYAHSQSLDRLMNPLIDQYLYYLSRTQTTSGTAGNRTLQFSQAGPSSMANQAKNWLPGPCYRQQRVSKTTNQNNNSNFAWTGATKYHLNGRDSLVNPGPAMATHKDDEDKFFPMSGVLIFGKQGAGNSNVDLDNVMITNEEEIKTTNPVATEEYGTVATNLQSANTAPATGTVNSQGALPGMVWQDRDVYLQGPIWAKIPHTDGHFHPSPLMGGFGLKHPPPQILIKNTPVPANPPTTFSPAKFASFITQYSTGQVSVEIEWELQKENSKRWNPEIQYTSNYNKSTNVDFAVDTNGVYSEPRPIGTRYLTRNL

SEQ ID NO: 23 - protein, 736 aa, synthetic construct; identical to SEQ ID NO: 22

SEQ ID NO: 24 - protein, 745 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVTTTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLALGETTRPARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSINVDFTVDTNGVYSEPRPIGTRYLTRNL

SEQ ID NO: 25 - DNA, 143 bp, Adeno-associated virus 1
  source 1..143: mol_type = genomic DNA
ttgcccactccctctctgcgcgctcgctcgctcggtggggcctgcggaccaaaggtccgcagacggcagagctctgctctgccggccccaccgagcgagcgagcgcgcagagagggagtgggcaactccatcactaggggtaa

SEQ ID NO: 26 - DNA, 143 bp, Adeno-associated virus 1
  source 1..143: mol_type = genomic DNA
ttacccctagtgatggagttgcccactccctctctgcgcgctcgctcgctcggtggggcctgcggaccaaaggtccgcagacggcagagctctgctctgccggccccaccgagcgagcgagcgcgcagagagggagtgggcaa

SEQ ID NO: 27 - DNA, 145 bp, Adeno-associated virus 2
  source 1..145: mol_type = genomic DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 28 - DNA, 145 bp, Adeno-associated virus 2
  source 1..145: mol_type = genomic DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 29 - DNA, 143 bp, Adeno-associated virus 3
  source 1..143: mol_type = genomic DNA
ttggccactccctctatgcgcactcgctcgctcggtggggcctggcgaccaaaggtcgccagacggacgtgctttgcacgtccggccccaccgagcgagcgagtgcgcatagagggagtggccaactccatcactagaggtat

SEQ ID NO: 30 - DNA, 143 bp, Adeno-associated virus 3
  source 1..143: mol_type = genomic DNA
atacctctagtgatggagttggccactccctctatgcgcactcgctcgctcggtggggcctggcgaccaaaggtcgccagacggacgtgctttgcacgtccggccccaccgagcgagcgagtgcgcatagagggagtggccaa

SEQ ID NO: 31 - DNA, 146 bp, Adeno-associated virus 4
  source 1..146: mol_type = genomic DNA
ttggccactccctctatgcgcgctcgctcactcactcggccctggagaccaaaggtctccagactgccggcctctggccggcagggccgagtgagtgagcgagcgcgcatagagggagtggccaactccatcatctaggtttgccc

SEQ ID NO: 32 - DNA, 144 bp, Adeno-associated virus 4
  source 1..144: mol_type = genomic DNA
ggcaaaccagatgatggagttggccacattagctatgcgcgctcgctcactcactcggccctggagaccaaaggtctccagactgccggcctctggccggcagggccgagtgagtgagcgagcgcgcatagagggagtggccaa

SEQ ID NO: 33 - DNA, 167 bp, Adeno-associated virus 5
  source 1..167: mol_type = genomic DNA
ctctcccccctgtcgcgttcgctcgctcgctggctcgtttgggggggtggcagctcaaagagctgccagacgacggccctctggccgtcgcccccccaaacgagccagcgagcgagcgaacgcgacaggggggagagtgccacactctcaagcaagggggttttgta

SEQ ID NO: 34 - DNA, 167 bp, Adeno-associated virus 5
  source 1..167: mol_type = genomic DNA
tacaaaacctccttgcttgagagtgtggcactctcccccctgtcgcgttcgctcgctcgctggctcgtttgggggggtggcagctcaaagagctgccagacgacggccctctggccgtcgcccccccaaacgagccagcgagcgagcgaacgcgacaggggggagag

SEQ ID NO: 35 - DNA, 145 bp, Adeno-associated virus 6; identical to SEQ ID NO: 27
  source 1..145: mol_type = genomic DNA

SEQ ID NO: 36 - DNA, 143 bp, Adeno-associated virus 6
  source 1..143: mol_type = genomic DNA
atacccctagtgatggagttgcccactccctctatgcgcgctcgctcgctcggtggggccggcagagcagagctctgccgtctgcggacctttggtccgcaggccccaccgagcgagcgagcgcgcatagagggagtgggcaa

SEQ ID NO: 37 - DNA, 145 bp, Adeno-associated virus 7
  source 1..145: mol_type = genomic DNA
ttggccactccctctatgcgcgctcgctcgctcggtggggcctgcggaccaaaggtccgcagacggcagagctctgctctgccggccccaccgagcgagcgagcgcgcatagagggagtggccaactccatcactaggggtaccg

SEQ ID NO: 38 - DNA, 145 bp, Adeno-associated virus 7
  source 1..145: mol_type = genomic DNA
cggtacccctagtgatggagttggccactccctctatgcgcgctcgctcgctcggtggggcctgcggaccaaaggtccgcagacggcagagctctgctctgccggccccaccgagcgagcgagcgcgcatagagggagtggccaa

SEQ ID NO: 39 - DNA, 80 bp, synthetic construct
  source 1..80: mol_type = other DNA
gccacggcggaactcatcgccgcctgccttgcccgctgctggacaggggctcggctgttgggcactgacaattccgtggt

SEQ ID NO: 40 - DNA, 145 bp, synthetic construct
  source 1..145: mol_type = other DNA
  misc_difference 70: note = a, c, g or t
ttggccactccctcthtgcgcgctcgctcgctcgctggggcctggvgaccaaaggtcgccagacggccgngctctgcccggccggccccagcgagcgagcgagcgcgcadagagggagtggccaactccatcactaggggtdccb

SEQ ID NO: 41 - DNA, 144 bp, synthetic construct
  source 1..144: mol_type = other DNA
  misc_difference 89: note = a, c, g or t
rdtacccctagtgatggagttggccactccctctatgcgcgctcgctcgctcggtggggcctgcvgaccaaaggtcbccagacggcvgngctctgchcggccggccccaccgagcgagcgagcgcgcatagagggagtggccaa

SEQ ID NO: 42 - DNA, 22 bp, Adeno-associated virus 2
  source 1..22: mol_type = genomic DNA
ctgcgcgctcgctcgctcactg

SEQ ID NO: 43 - DNA, 22 bp, Adeno-associated virus 2
  source 1..22: mol_type = genomic DNA
cagtgagcgagcgagcgcgcag

SEQ ID NO: 44 - protein, 4 aa, synthetic construct
GSSS

SEQ ID NO: 45 - protein, 5 aa, synthetic construct
GSSSS

SEQ ID NO: 46 - protein, 6 aa, synthetic construct
GSSGSS

SEQ ID NO: 47 - skipped

SEQ ID NO: 48 - DNA, 387 bp, synthetic construct
  source 1..387: mol_type = other DNA; note = MCP nucleic acid sequence
gcttctaactttactcagttcgttctcgtcgacaatggcggaactggcgacgtgactgtcgccccaagcaacttcgctaacggggtcgctgaatggatcagctctaactcgcgttcacaggcttacaaagtaacctgtagcgttcgtcagagctctgcgcagaatcgcaaatacaccatcaaagtcgaggtgcctaaagtggcaacccagactgttggtggagtagagcttcctgtagccgcatggcgttcgtacttaaatatggaactaaccattccaattttcgctacgaattccgactgcgagcttattgttaaggcaatgcaaggtctcctaaaagatggaaacccgattccctcagcaatcgcagcaaactccggcatctac

SEQ ID NO: 49 - protein, 129 aa, synthetic construct
  source 1..129: note = MCP nucleic acid sequence
ASNFTQFVLVDNGGTGDVTVAPSNFANGVAEWISSNSRSQAYKVTCSVRQSSAQNRKYTIKVEVPKVATQTVGGVELPVAAWRSYLNMELTIPIFATNSDCELIVKAMQGLLKDGNPIPSAIAANSGIY

SEQ ID NO: 50 - DNA, 363 bp, synthetic construct
  source 1..363: mol_type = other DNA; note = PCP nucleic acid sequence
tccaaaacaatagtcctctccgtaggggaggcaacacggactttgaccgaaatccagtcaaccgctgaccgacaaatctttgaagagaaagtagggcctcttgtgggccgactgcgcttgactgcaagcttgcgacaaaacggcgcaaagactgcctatagggtcaaccttaaactcgaccaagccgacgtggtcgatagcggtctccctaaggttcggtatacgcaggtctggagtcatgacgtaacaatcgtagcaaacagcacagaagcctcccgaaaaagcctctacgatctgacgaaatccttggtggctacgtcacaggtggaagacctcgttgtcaaccttgtacctctgggtcga

SEQ ID NO: 51 - protein, 121 aa, synthetic construct
  source 1..121: note = PCP nucleic acid sequence
SKTIVLSVGEATRTLTEIQSTADRQIFEEKVGPLVGRLRLTASLRQNGAKTAYRVNLKLDQADVVDSGLPKVRYTQVWSHDVTIVANSTEASRKSLYDLTKSLVATSQVEDLVVNLVPLGR

SEQ ID NO: 52 - DNA, 186 bp, synthetic construct
  source 1..186: mol_type = other DNA; note = COM nucleic acid sequence
atgaaatcaattcgctgtaaaaactgcaacaaactgttatttaaggcggattcctttgatcacattgaaatcaggtgtccgcgttgcaaacgtcacatcataatgctgaatgcctgcgagcatcccacggagaaacattgtgggaaaagagaaaaaatcacgcattctgacgaaaccgtgcgttat

SEQ ID NO: 53 - protein, 62 aa, synthetic construct
  source 1..62: note = COM nucleic acid sequence
MKSIRCKNCNKLLFKADSFDHIEIRCPRCKRHIIMLNACEHPTEKHCGKREKITHSDETVRY

SEQ ID NO: 54 - RNA, 19 nt, synthetic construct
  source 1..19: mol_type = other RNA; note = MS2
acatgaggatcacccatgt

SEQ ID NO: 55 - RNA, 128 nt, synthetic construct
  source 1..128: mol_type = other RNA; note = MS2 X3(MS2-linker-MS2-linker-MS2)
acatgaggatcacccatgtctgcaggtcgactctagaaaacatgaggatcacccatgtctgcagtattcccgggttcattagatcctaaggtacctaattgcctagaaaacatgaggatcacccatgt

SEQ ID NO: 56 - RNA, 25 nt, synthetic construct
  source 1..25: mol_type = other RNA; note = PP7
ggagcagacgatatggcgtcgctcc

SEQ ID NO: 57 - RNA, 146 nt, synthetic construct
  source 1..146: mol_type = other RNA; note = PP7 X3(PP7-linker-PP7-linker-PP7)
ggagcagacgatatggcgtcgctccctgcaggtcgactctagaaaggagcagacgatatggcgtcgctccctgcagtattcccgggttcattagatcctaaggtacctaattgcctagaaaggagcagacgatatggcgtcgctcc

SEQ ID NO: 58 - RNA, 18 nt, synthetic construct
  source 1..18: mol_type = other RNA; note = com
gaatgcctgcgagcatcc

SEQ ID NO: 59 - RNA, 125 nt, synthetic construct
  source 1..125: mol_type = other RNA; note = com X3(com-linker-com-linker-com)
gaatgcctgcgagcatccctgcaggtcgactctagaaagaatgcctgcgagcatccctgcagtattcccgggttcattagatcctaaggtacctaattgcctagaaagaatgcctgcgagcatcc

SEQ ID NO: 60 - DNA, 1866 bp, synthetic construct
  source 1..1866: mol_type = other DNA; note = Rep2 nucleic acid sequence
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 61 - protein, 621 aa, synthetic construct
  source 1..621: note = Rep2 amino acid sequence
MPGFYEIVIKVPSDLDEHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 62 - DNA, 2289 bp, synthetic construct
  source 1..2289: mol_type = other DNA; note = MCP-Rep nucleic acid sequence (linker-MCP-linker-Rep)
atgcccggcagctccggcagtagcgcttctaactttactcagttcgttctcgtcgacaatggcggaactggcgacgtgactgtcgccccaagcaacttcgctaacggggtcgctgaatggatcagctctaactcgcgttcacaggcttacaaagtaacctgtagcgttcgtcagagctctgcgcagaatcgcaaatacaccatcaaagtcgaggtgcctaaagtggcaacccagactgttggtggagtagagcttcctgtagccgcatggcgttcgtacttaaatatggaactaaccattccaattttcgctacgaattccgactgcgagcttattgttaaggcaatgcaaggtctcctaaaagatggaaacccgattccctcagcaatcgcagcaaactccggcatctacggcagtagtgggtcctctgggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 63 - protein, 762 aa, synthetic construct
  source 1..762: note = MCP-Rep amino acid sequence(linker-MCP-linker-Rep)
MPGSSGSSASNFTQFVLVDNGGTGDVTVAPSNFANGVAEWISSNSRSQAYKVTCSVRQSSAQNRKYTIKVEVPKVATQTVGGVELPVAAWRSYLNMELTIPIFATNSDCELIVKAMQGLLKDGNPIPSAIAANSGIYGSSGSSGFYEIVIKVPSDLDEHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 64 - DNA, 2289 bp, synthetic construct
  source 1..2289: mol_type = other DNA; note = MCP-Rep-Y156F nucleic acid sequence (linker-MCP-linker-Rep)
atgcccggcagctccggcagtagcgcttctaactttactcagttcgttctcgtcgacaatggcggaactggcgacgtgactgtcgccccaagcaacttcgctaacggggtcgctgaatggatcagctctaactcgcgttcacaggcttacaaagtaacctgtagcgttcgtcagagctctgcgcagaatcgcaaatacaccatcaaagtcgaggtgcctaaagtggcaacccagactgttggtggagtagagcttcctgtagccgcatggcgttcgtacttaaatatggaactaaccattccaattttcgctacgaattccgactgcgagcttattgttaaggcaatgcaaggtctcctaaaagatggaaacccgattccctcagcaatcgcagcaaactccggcatctacggcagtagtgggtcctctgggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaatttcttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 65 - protein, 762 aa, synthetic construct
  source 1..762: note = MCP-Rep-Y156F amino acid sequence (linker-MCP-linker-Rep)
MPGSSGSSASNFTQFVLVDNGGTGDVTVAPSNFANGVAEWISSNSRSQAYKVTCSVRQSSAQNRKYTIKVEVPKVATQTVGGVELPVAAWRSYLNMELTIPIFATNSDCELIVKAMQGLLKDGNPIPSAIAANSGIYGSSGSSGFYEIVIKVPSDLDEHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNFLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 66 - DNA, 2289 bp, synthetic construct
  source 1..2289: mol_type = other DNA; note = MCP-Rep-KDE-mu nucleic acid sequence (linker-MCP-linker-Rep)
atgcccggcagctccggcagtagcgcttctaactttactcagttcgttctcgtcgacaatggcggaactggcgacgtgactgtcgccccaagcaacttcgctaacggggtcgctgaatggatcagctctaactcgcgttcacaggcttacaaagtaacctgtagcgttcgtcagagctctgcgcagaatcgcaaatacaccatcaaagtcgaggtgcctaaagtggcaacccagactgttggtggagtagagcttcctgtagccgcatggcgttcgtacttaaatatggaactaaccattccaattttcgctacgaattccgactgcgagcttattgttaaggcaatgcaaggtctcctaaaagatggaaacccgattccctcagcaatcgcagcaaactccggcatctacggcagtagtgggtcctctgggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacgcggtggtggctgcgtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 67 - protein, 762 aa, synthetic construct
  source 1..762: note = MCP-Rep-KDE-muamino acid sequence (linker-MCP-linker-Rep)
MPGSSGSSASNFTQFVLVDNGGTGDVTVAPSNFANGVAEWISSNSRSQAYKVTCSVRQSSAQNRKYTIKVEVPKVATQTVGGVELPVAAWRSYLNMELTIPIFATNSDCELIVKAMQGLLKDGNPIPSAIAANSGIYGSSGSSGFYEIVIKVPSDLDEHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNAVVAACYIPNYLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 68 - DNA, 2289 bp, synthetic construct
  source 1..2289: mol_type = other DNA; note = MCP-Rep-EKE-mu nucleic acid sequence (linker-MCP-linker-Rep)
atgcccggcagctccggcagtagcgcttctaactttactcagttcgttctcgtcgacaatggcggaactggcgacgtgactgtcgccccaagcaacttcgctaacggggtcgctgaatggatcagctctaactcgcgttcacaggcttacaaagtaacctgtagcgttcgtcagagctctgcgcagaatcgcaaatacaccatcaaagtcgaggtgcctaaagtggcaacccagactgttggtggagtagagcttcctgtagccgcatggcgttcgtacttaaatatggaactaaccattccaattttcgctacgaattccgactgcgagcttattgttaaggcaatgcaaggtctcctaaaagatggaaacccgattccctcagcaatcgcagcaaactccggcatctacggcagtagtgggtcctctgggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgcggcgggagcgagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 69 - protein, 762 aa, synthetic construct
  source 1..762: note = MCP-Rep-EKE-muamino acid sequence (linker-MCP-linker-Rep)
MPGSSGSSASNFTQFVLVDNGGTGDVTVAPSNFANGVAEWISSNSRSQAYKVTCSVRQSSAQNRKYTIKVEVPKVATQTVGGVELPVAAWRSYLNMELTIPIFATNSDCELIVKAMQGLLKDGNPIPSAIAANSGIYGSSGSSGFYEIVIKVPSDLDEHLPGISDSFVNWVAEKEWELPPDSDMDLNLIEQAPLTVAEKLQRDFLTEWRRVSKAPEALFFVQFAAGASYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 70 - DNA, 2616 bp, synthetic construct
  source 1..2616: mol_type = other DNA; note = 2XMCP-Rep nucleic acid sequence (linker-2XMCP-linker-Rep)
atgcccggcagctccggcagtagcgcttctaactttactcagttcgttctcgtcgacaatggcggaactggcgacgtgactgtcgccccaagcaacttcgctaacgggatcgctgaatggatcagctctaactcgcgttcacaggcttacaaagtaacctgtagcgttcgtcagagctctgcgcagaatcgcaaatacaccatcaaagtcgaggtgcctaaaggcgcctggcgttcgtacttaaatatggaactaaccattccaattttcgccacgaattccgactgcgagcttattgttaaggcaatgcaaggtctcctaaaagatggaaacccgattccctcagcaatcgcagcaaactccggcatctacggtggtggaggaggaatggcgtccaatttcacgcagttcgtcctggttgacaacggggggactggggacgttacggtcgctccgagcaactttgccaatggtattgcggagtggatttcttctaattcacggtcccaagcttacaaagtgacctgttccgtgcggcaaagttctgctcagaatagaaagtacactataaaggtcgaagtccctaagggggcctggcgatcatatctcaatatggagcttaccatcccaatatttgccactaattctgattgtgaattgattgtcaaagcaatgcaaggactcttgaaagacggaaacccaatccccagcgcaatcgcagccaactccggtatatacggcagtagtgggtcctctgggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 71 - protein, 871 aa, synthetic construct
  source 1..871: note = 2XMCP-Rep amino acid sequence (linker-2XMCP-linker-Rep)
MPGSSGSSASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGGGGMASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGSSGSSGFYEIVIKVPSDLDEHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 72 - DNA, 2265 bp, synthetic construct
  source 1..2265: mol_type = other DNA; note = PCP-Rep-Y156F nucleic acid sequence (linker-PCP-linker-Rep)
atgcccggcagctccggcagtagctccaaaacaatagtcctctccgtaggggaggcaacacggactttgaccgaaatccagtcaaccgctgaccgacaaatctttgaagagaaagtagggcctcttgtgggccgactgcgcttgactgcaagcttgcgacaaaacggcgcaaagactgcctatagggtcaaccttaaactcgaccaagccgacgtggtcgatagcggtctccctaaggttcggtatacgcaggtctggagtcatgacgtaacaatcgtagcaaacagcacagaagcctcccgaaaaagcctctacgatctgacgaaatccttggtggctacgtcacaggtggaagacctcgttgtcaaccttgtacctctgggtcgaggcagtagtgggtcctctgggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaatttcttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 73 - protein, 754 aa, synthetic construct
  source 1..754: note = PCP-Rep-Y156F amino acid sequence (linker-PCP-linker-Rep)
MPGSSGSSSKTIVLSVGEATRTLTEIQSTADRQIFEEKVGPLVGRLRLTASLRQNGAKTAYRVNLKLDQADVVDSGLPKVRYTQVWSHDVTIVANSTEASRKSLYDLTKSLVATSQVEDLVVNLVPLGRGSSGSSGFYEIVIKVPSDLDEHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNFLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 74 - DNA, 2088 bp, synthetic construct
  source 1..2088: mol_type = other DNA; note = COM-Rep-Y156F nucleic acid sequence (linker-COM-linker-Rep)
atgcccggcagctccggcagtagcatgaaatcaattcgctgtaaaaactgcaacaaactgttatttaaggcggattcctttgatcacattgaaatcaggtgtccgcgttgcaaacgtcacatcataatgctgaatgcctgcgagcatcccacggagaaacattgtgggaaaagagaaaaaatcacgcattctgacgaaaccgtgcgttatggcagtagtgggtcctctgggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaatttcttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 75 - protein, 695 aa, synthetic construct
  source 1..695: note = COM-Rep-Y156F amino acid sequence (linker-COM-linker-Rep)
MPGSSGSSMKSIRCKNCNKLLFKADSFDHIEIRCPRCKRHIIMLNACEHPTEKHCGKREKITHSDETVRYGSSGSSGFYEIVIKVPSDLDEHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNFLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 76 - DNA, 1020 bp, synthetic construct
  source 1..1020: mol_type = other DNA; note = MCP-AAP(DJ) nucleic acid sequence (linker-MCP-linker-AAP)
atgggcagctccggcagtagcgcttctaactttactcagttcgttctcgtcgacaatggcggaactggcgacgtgactgtcgccccaagcaacttcgctaacggggtcgctgaatggatcagctctaactcgcgttcacaggcttacaaagtaacctgtagcgttcgtcagagctctgcgcagaatcgcaaatacaccatcaaagtcgaggtgcctaaagtggcaacccagactgttggtggagtagagcttcctgtagccgcatggcgttcgtacttaaatatggaactaaccattccaattttcgctacgaattccgactgcgagcttattgttaaggcaatgcaaggtctcctaaaagatggaaacccgattccctcagcaatcgcagcaaactccggcatctacggcagtagtgggtcctctctggagacgcagactcagtcccagaccctcaaccaatcggagaacctcccgcagccccctcaggtgtgggatctcttacaatggctgcaggcggtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaagcaaatctccaacagcacatctggaggatcttcaaatgacaacgcctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatga

SEQ ID NO: 77 - protein, 339 aa, synthetic construct
  source 1..339: note = MCP-AAP(DJ) amino acid sequence (linker-MCP-linker-AAP)
MGSSGSSASNFTQFVLVDNGGTGDVTVAPSNFANGVAEWISSNSRSQAYKVTCSVRQSSAQNRKYTIKVEVPKVATQTVGGVELPVAAWRSYLNMELTIPIFATNSDCELIVKAMQGLLKDGNPIPSAIAANSGIYGSSGSSLETQTQSQTLNQSENLPQPPQVWDLLQWLQAVAHQWQTITRAPTEWVIPREIGIAIPHGWATESSPPAPEPGPCPPTTTTSTSKSPTAHLEDLQMTTPTSATAPPGGILTLTDSTATFHHVTGSDSSTTTGDSGPRDSASSSSTSRSRRSRRMKAPRPSPITSPAPSRCLRTRSTSCRTFSALPTRAACLRSRRTCS

SEQ ID NO: 78 - DNA, 1020 bp, synthetic construct
  source 1..1020: mol_type = other DNA; note = AAP-MCP(DJ) nucleic acid sequence (AAP-linker-MCP-linker)
atgctggagacgcagactcagtcccagaccctcaaccaatcggagaacctcccgcagccccctcaggtgtgggatctcttacaatggctgcaggcggtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaagcaaatctccaacagcacatctggaggatcttcaaatgacaacgcctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcaggcagctccggcagtagcgcttctaactttactcagttcgttctcgtcgacaatggcggaactggcgacgtgactgtcgccccaagcaacttcgctaacggggtcgctgaatggatcagctctaactcgcgttcacaggcttacaaagtaacctgtagcgttcgtcagagctctgcgcagaatcgcaaatacaccatcaaagtcgaggtgcctaaagtggcaacccagactgttggtggagtagagcttcctgtagccgcatggcgttcgtacttaaatatggaactaaccattccaattttcgctacgaattccgactgcgagcttattgttaaggcaatgcaaggtctcctaaaagatggaaacccgattccctcagcaatcgcagcaaactccggcatctacggcagtagtgggtcctcttga

SEQ ID NO: 79 - protein, 339 aa, synthetic construct
  source 1..339: note = AAP-MCP(DJ) amino acid sequence (AAP-linker-MCP-linker)
MLETQTQSQTLNQSENLPQPPQVWDLLQWLQAVAHQWQTITRAPTEWVIPREIGIAIPHGWATESSPPAPEPGPCPPTTTTSTSKSPTAHLEDLQMTTPTSATAPPGGILTLTDSTATFHHVTGSDSSTTTGDSGPRDSASSSSTSRSRRSRRMKAPRPSPITSPAPSRCLRTRSTSCRTFSALPTRAACLRSRRTCSGSSGSSASNFTQFVLVDNGGTGDVTVAPSNFANGVAEWISSNSRSQAYKVTCSVRQSSAQNRKYTIKVEVPKVATQTVGGVELPVAAWRSYLNMELTIPIFATNSDCELIVKAMQGLLKDGNPIPSAIAANSGIYGSSGSS

SEQ ID NO: 80 - DNA, 23 bp, synthetic construct
  source 1..23: mol_type = other DNA; note = WPRE-F
cccgtatggctttcattttctcc

SEQ ID NO: 81 - DNA, 19 bp, synthetic construct
  source 1..19: mol_type = other DNA; note = WPRE-R
ggcaatgccccaaccagtg

SEQ ID NO: 82 - DNA, 20 bp, synthetic construct
  source 1..20: mol_type = other DNA; note = Cre-F
ccagtagatgccactagcga

SEQ ID NO: 83 - DNA, 20 bp, synthetic construct
  source 1..20: mol_type = other DNA; note = Cre-R
gcctggagatacagcaggta

SEQ ID NO: 84 - DNA, 20 bp, synthetic construct
  source 1..20: mol_type = other DNA; note = CAG-F
cttctcctccgggctgtaat

SEQ ID NO: 85 - DNA, 20 bp, synthetic construct
  source 1..20: mol_type = other DNA; note = CAG-R
ctttcacgcagccacagaaa

SEQ ID NO: 86 - DNA, 3266 bp, synthetic construct
  source 1..3266: mol_type = other DNA; note = Stuffer nucleic acid sequence
atatttggagggcagcttgatttcgacttcgggagggaagctgcgccatgcgatgttatcggtgcggtgaatgcaaagaagataaccgcttccgaccaaatcaaccttactggaatcgatggtgtctccggtgtgaaagaacaccaacaggggtgttaccactaccgcaggaaaaggaggacgtgccgcgagacagcgacgaagtatcaccgacataatctgcgaaaactgcaaataccttccaacgaaacgcaccagaaataaacccaagccaatcccaaaagaatctgacgtaaaaaccttcaactacacggctcacctgtgggatatccggtggctaagacgtcgtgcgaggaaaacaaggccattgaccaaaatcgaagttacgaacaagaaagcgtcgagcgagctttaacgtgcgctaactgcggtcagaagctgcatgtgctggaagttcacgtgtgtgagcactgctgcgcagaactgatgagcgatccgaatagctcgatgcacgaggaagaaggccgccgctaaaccagcgcgaagacgatgtaaaaacgatgaatgccgggaatggtttcaccctgcattcgctaatcagtggtggtgctctccagagtgtggaaccaagatagcactcgaacgacgaagtaaagaacgcgaaaaagcggaaaaagcagcagagaagaaacgacgacgagaggagcagaaacagaaagataaacttaagattcgaaaactcgccttaaagccccgcagttactggattaaacaagcccaacaagccgtaaacgccttcatcagagaaagagaccgcgacttaccatgtatctcgtgcggaacgctcacgtctgctcagtgggatgccggacattaccggacaactgctgcggcacctcaactccgatttaatgaacgcaatattcacaagcaatgcgtggtgtgcaaccagcacaaaagcggaaatctcgttccgtatcgcgtcgaactgattagccgcatcgggcaggaagcagtagacgaaatcgaatcaaaccataaccgccatcgctggactatcgaagagtgcaaggcgatcaaggcagagtaccaacagaaactcaaagacctgcgaaatagcagaagtgaggccgcgccacgttctcagtaaaaaccattccagacatgctcgttgaagcatacggaaatcagacagaagtagcacgcagactgaaatgtagtcgcggtacggtcagaaaatacgttgatgataaagacgggaaaatgcacgccatcgtcaacgacgttctcatggttcatcgcggatggagtgaaagaggcccgctattacgaaaaaattgatggcagcaaataccgaaatatttgggtagttggcgatctgcacggatgctacacgaacctgatgaacaaactggatacgattggattcgacaacaaaaaagacctgcttatctcggtgggcgatttggttgatcgtggtgcagagaacgttgaatgcctggaattaatcacattcccctggttcagagctgtacgtggaaaccatgagcaaatgatgattgatggcttatcagagcgtggaaacgttaatcactggctgcttaatggcggtggctggttctttaatctcgattacgacaaagaaattctggctaaagctcttgcccataaagcagatgaacttccgttaatcatcgaactggtgagcaaagataaaaaatatgttatctgccacgccgattatccctttgacgaatacgagtttggaaagccagttgatcatcagcaggtaatctggaaccgcgaacgaatcagcaactcacaaaacgggatcgtgaaagaaatcaaaggcgcggacacgttcatctttggtcatacgccagcagtgaaaccactcaagtttgccaaccaaatgtatatcgataccggcgcagtgttctgcggaaacctaacattgattcaggtacagggagaaggcgcgccagactcgaaagcgtagctaaatttcattcgccaaaaagcccgatgatgagcgactcaccacgggccacggcttctgactctctttccggtactgatgtgatggctgctatggggatggcgcaatcacaagccggattcggtatggctgcattctgcggtaagcacgaactcagccagaacgacaaacaaaaggctatcaactatctgatgcaatttgcacacaaggtatcggggaaataccgtggtgtggcaaagcttgaaggaaatactaaggcaaaggtactgcaagtgctcgcaacattcgcttatgcggattattgccgtagtgccgcgacgccgggggcaagatgcagagattgccatggtacaggccgtgcggttgatattgccaaaacagagctgtgggggagagttgtcgagaaagagtgcggaagatgcaaaggcgtcggctattcaaggatgccagcaagcgcagcatatcgcgctgtgacgatgctaatcccaaaccttacccaacccacctggtcacgcactgttaagccgctgtatgacgctctggtggtgcaatgccacaaagaagagtcaatcgcagacaacattttgaatgcggtcacacgttagcagcatgattgccacggatggcaacatattaacggcatgatattgacttattgaataaaattgggtaaatttgactcaacgatgggttaattcgctcgttgtggtagtgaggccaaaagaggcggcgcttactaccgattccgcctagttggtcacttcgacgtatcgtctggaactccaaccatcgcaggcagagaggtctgcaaaatgcaatcccgaaacagttcgcaggtaatagttagagcctgcataacggtttcgggattttttatatctgcacaacaggtaagagcattgagtcgataatcgtgaagagtcggcgagcctggttagccagtgctctttccgttgtgctgaattaagcgaataccggaagcagaaccggatcaccaaatgcgtacaggcgtcatcgccgcccagcaacagcacaacccaaactgagccgtagccactgtctgtcctaaattcattagtaatagttacgctgcggccttttacacatgaccttcgtgaaagcgggtggcaggaggtcgcgctaacaacctcctgccgttttgcccgtgcatatcggtcacgaacaaatctgattactaaacacagtagcctggatttgttctatcagtaatcgaccttattcctaattaaatagagcaaatcccctt

SEQ ID NO: 87 - protein, 120 aa, synthetic construct
  VARIANT 1..120: note = This sequence may encompass 1-20 (Gly-Ser)p repeats where p is 1-5
GSSSSSGSSSSSGSSSSSGSSSSSGSSSSSGSSSSSGSSSSSGSSSSSGSSSSSGSSSSSGSSSSSGSSSSSGSSSSSGSSSSSGSSSSSGSSSSSGSSSSSGSSSSSGSSSSSGSSSSS

SEQ ID NO: 88 - protein, 621 aa, synthetic construct; identical to SEQ ID NO: 61

SEQ ID NO: 89 - protein, 671 aa, synthetic construct
MELFRGVLQVSSNVLDCANDNWWCSLLDLDTSDWEPLTHTNRLMAIYLSSVASKLDFTGGPLAGCLYFFQVECNKFEEGYHIHVVIGGPGLNPRNLTVCVEGLFNNVLYHLVTENVKLKFLPGMTTKGKYFRDGEQFIENYLMKKIPLNVVWCVTNIDGYIDTCISATFRRGACHAKKPRITTAINDTSSDAGESSGTGAEVVPINGKGTKASIKFQTMVNWLCENRVFTEDKWKLVDFNQYTLLSSSHSGSFQIQSALKLAIYKATNLVPTSTFLLHTDFEQVMCIKDNKIVKLLLCQNYDPLLVGQHVLKWIDKKCGKKNTLWFYGPPSTGKTNLAMAIAKSVPVYGMVNWNNENFPFNDVAGKSLVVWDEGIIKSTIVEAAKAILGGQPTRVDQKMRGSVAVPGVPVVITSNGDITFVVSGNTTTTVHAKALKERMVKLNFTVRCSPDMGLLTEADVQQWLTWCNAQSWDHYENWAINYTFDFPGINADALHPDLQTTPIVTDTSISSSGGESSEELSESSFFNLITPGAWNTETPRSSTPIPGTSSGESFVGSSVSSEVVAASWEEAFYTPLADQFRELLVGVDYVWDGVRGLPVCCVQHINNSGGGLGLCPHCINVGAWYNGWKFREFTPDLVRCSCHVGASNPFSVLTCKKCAYLSGLQSFVDYE

SEQ ID NO: 90 - protein, 671 aa, synthetic construct
MELFRGVLQVSSNVLDCANDNWWCSLLDLDTSDWEPLTHTNRLMAIYLSSVASKLDLTGGPLAGCLYFFQAECNKFEEGYHIHVVIGGPGLNPRNLTVCVEGLFNNVLYHFVTENVKLKFLPGMTTKGKYFRDGEQFIENYLMKKIPLNVVWCVTNIDGYIDTCISATFRRGACHAKKPRITTAINDTSSDAGESSGTGAEVVPFNGKGTKASIKFQTMVNWLCENRVFTEDKWKLVDFNQYTLLSSSHSGSFQIQSALKLAIYKATNLVPTSTFLLHTDFEQVMCIKDNKIVKLLLCQNYDPLLVGQHVLKWIDKKCGKKNTLWFYGPPSTGKTNLAMAIAKSVPVYGMVNWNNENFPFNDVAGKSLVVWDEGIIKSTIVEAAKAILGGQPTRVDQKMRGSVAVPGVPVVITSNGDITFVVSGNTTTTVHAKALKERMVKLNFTVRCSPDMGLLTEADVQQWLTWCNAQSWDHYENWAINYTFDFPGINADALHPDLQTTPIVTDTSISSSGGESSEELSESSFFNLITPGAWNTETPRSSTPIPGTSSGESFVGSPVSSEVVAASWEEAFYTPLADQFRELLVGVDYVWDGVRGLPVCCVQHINNSGGGLGLCPHCINVGAWYNGWKFREFTPDLVRCSCHVGASNPFSVLTCKKCAYLSGLQSFVDYE

SEQ ID NO: 91 - protein, 622 aa, synthetic construct
MPGFYEIVIKVPSDLDEHLPGISDSFVNWVAEKEWELPPDSDMDRNLIEQAPLTVAEKLQRDFLVHWRRVSKAPEALFFVQFEKGESYFHLHVLVETTGVKSMVLGRFLSQIRDRLVQTIYRGVEPTLPNWFAVTKTRNGAGGGNKVVDECYIPNYLLPKTQPELQWAWTNMEEYISACLNLAERKRLVAQHLTHVSQTQEQNKENLNPNSDAPVIRSKTSARYMELVGWLVDRGITSEKQWIQEDQASYISFNAASNSRSQIKAALDNAGKIMALTKSAPDYLVGPSLPADIKANRIYRILELNGYDPAYAGSVFLGWAQKKFGKRNTIWLFGPATTGKTNIAEAIAHAVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLEHDFGKVTKQEVKEFFRWAQDHVTEVTHEFYVRKGGATKRPAPSDADISEPKRACPSVAEPSTSDAEAPVDFADRYQNKCSRHAGMLQMLFPCKTCERMNQNFNVCFTHGVRDCSECFPGASESQPVVRKKTYQKLCAIHHLLGRAPEIACSACDLVNVDLDDCVSEQ

SEQ ID NO: 92 - protein, 622 aa, synthetic construct
MPGFYEIVIKVPSDLDEHLPGISDSFVNWVAEKEWELPPDSDMDRNLIEQAPLTVAEKLQRDFLVHWRRVSKAPEALFFVQFEKGESYFHLHVLVETTGVKSMVLGRFLSQIRDRLVQTIYRGVEPTLPNWFAVTKTRNGAGGGNKVVDECYIPNYLLPKTQPELQWAWTNMEEYISACLNLAERKRLVAQHLTHVSQTQEQNKENLNPNSDAPVIRSKTSARYMELVGWLVDRGITSEKQWIQEDQASYISFNAASNSRSQIKAALDNAGKIMALTKSAPDYLVGPSLPADIKANRIYRILELNGYDPAYAGSVFLGWAQKKFGKRNTIWLFGPATTGKTNIAEAIAHAVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLEHDFGKVTKQEVKEFFRWAQDHVTEVAHEFYVRKGGATKRPAPSDADISEPKRACPSVPEPSTSDAEAPVDFADRYQNKCSRHAGMLQMLFPCKTCERMNQNFNVCFTHGVRDCSECFPGASESQPVVRKKTYQKLCAIHHLLGRAPEIACSACDLVNVDLDDCVSEQ

SEQ ID NO: 93 - protein, 623 aa, synthetic construct
MPGFYEIVIKVPSDLDEHLPGISDSFVNWVAEKEWELPPDSDMDLNLIEQAPLTVAEKLQRDFLVQWRRVSKAPEALFFVQFEKGESYFHLHVLVETTGVKSMVLGRFLSQIREKLVQTIYRGVEPTLPNWFAVTKTRNGAGGGNKVVDECYIPNYLLPKTQPELQWAWTNMEEYISACLNLAERKRLVAQHLTHVSQTQEQNKENLNPNSDAPVIRSKTSARYMELVGWLVDRGITSEKQWIQEDQASYISFNAASNSRSQIKAALDNAGKIMALTKSAPDYLVGPSLPADIKTNRIYRILELNGYDPAYAGSVFLGWAQKKFGKRNTIWLFGPATTGKTNIAEAIAHAVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLEHDFGKVTKQEVKEFFRWASDHVTEVAHEFYVRKGGASKRPAPDDADISEPKRACPSVADPSTSDAEGAPVDFADRYQNKCSRHAGMIQMLFPCKTCERMNQNFNICFTHGVRDCLECFPGVSESQPVVRKKTYRKLCAIHHLLGRAPEIACSACDLVNVDLDDCVSEQ

SEQ ID NO: 94 - protein, 625 aa, synthetic construct
MPGFYEIVIKVPSDLDEHLPGISDSFVNWVAEKEWELPPDSDMDRNLIEQAPLTVAEKLQRDFLVQWRRVSKAPEALFFVQFEKGESYFHLHVLVETTGVKSMVLGRFLSQIREKLGPDHLPAGSSPTLPNWFAVTKDAVMAPAGGNKVVDECYIPNYLLPKTQPELQWAWTNMEEYISACLNLAERKRLVAQHLTHVSQTQEQNKENLNPNSDAPVIRSKTSARYMELVGWLVDRGITSEKQWIQEDQASYISFNAASNSRSQIKAALDNAGKIMALTKSAPDYLVGPSLPADITQNRIYRILALNGYDPAYAGSVFLGWAQKKFGKRNTIWLFGPATTGKTNIAEAIAHAVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLEHDFGKVTKQEVKEFFRWASDHVTEVAHEFYVRKGGASKRPAPDDADKSEPKRACPSVADPSTSDAEGAPVDFADRYQNKCSRHAGMLQMLFPCKTCERMNQNFNICFTHGVRDCSECFPGVSESQPVVRKRTYRKLCAIHHLLGRAPEIACSACDLVNVDLDDCVSEQ

SEQ ID NO: 95 - protein, 623 aa, synthetic construct
MPGFYEIVIKVPSDLDEHLPGISDSFVNWVAEKEWELPPDSDMDRNLIEQAPLTVAEKLQRDFLVQWRRVSKAPEALFFVQFEKGESYFHLHVLVETTGVKSMVLGRFLSQIREKLVQTIYRGIEPTLPNWFAVTKTRNGAGGGNKVVDECYIPNYLLPKTQPELQWAWTNMEEYISACLNLAERKRLVAQHLTHVSQTQEQNKENLNPNSDAPVIRSKTSARYMELVGWLVDRGITSEKQWIQEDQASYISFNAASNSRSQIKAALDNAGKIMALTKSAPDYLVGPSLPVDITQNRIYRILQLNGYDPAYAGSVFLGWAQKKFGKRNTIWLFGPATTGKTNIAEAIAHAVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLEHDFGKVTKQEVKEFFRWASDHVTEVAHEFYVRKGGASKRPAPDDADKSEPKRACPSVADPSTSDAEGAPVDFADRYQNKCSRHAGMLQMLLPCKTCERMNQNFNICFTHGVRDCSECFPGVSESQPVVRKRTYRKLCAIHHLLGRAPEIACSACDLVNVDLDDCVSEQ

SEQ ID NO: 96 - protein, 623 aa, synthetic construct
MPGFYEIVIKVPSDLDEHLPGISDSFVSWVAEKEWELPPDSDMDLNLIEQAPLTVAEKLQRDFLVQWRRVSKAPEALFFVQFEKGESYFHLHILVETTGVKSMVLGRFLSQIRDKLVQTIYRGIEPTLPNWFAVTKTRNGAGGGNKVVDECYIPNYLLPKTQPELQWAWTNMEEYISACLNLAERKRLVAQHLTHVSQTQEQNKENLNPNSDAPVIRSKTSARYMELVGWLVDRGITSEKQWIQEDQASYISFNAASNSRSQIKAALDNAGKIMALTKSAPDYLVGPAPPADIKTNRIYRILELNGYEPAYAGSVFLGWAQKRFGKRNTIWLFGPATTGKTNIAEAIAHAVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLEHDFGKVTKQEVKEFFRWAQDHVTEVAHEFYVRKGGANKRPAPDDADKSEPKRACPSVADPSTSDAEGAPVDFADRYQNKCSRHAGMLQMLFPCKTCERMNQNFNICFTHGTRDCSECFPGVSESQPVVRKRTYRKLCAIHHLLGRAPEIACSACDLVNVDLDDCVSEQ

SEQ ID NO: 97 - protein, 623 aa, synthetic construct
MPGFYEIVIKVPSDLDEHLPGISDSFVNWVAEKEWELPPDSDMDLNLIEQAPLTVAEKLQRDFLVQWRRVSKAPEALFFVQFEKGESYFHLHILVETTGVKSMVLGRFLSQIRDKLVQTIYRGIEPTLPNWFAVTKTRNGAGGGNKVVDECYIPNYLLPKTQPELQWAWTNMEEYISACLNLAERKRLVAHDLTHVSQTQEQNKENLNPNSDAPVIRSKTSARYMELVGWLVDRGITSEKQWIQEDQASYISFNAASNSRSQIKAALDNAGKIMALTKSAPDYLVGPAPPADIKTNRIYRILELNGYDPAYAGSVFLGWAQKRFGKRNTIWLFGPATTGKTNIAEAIAHAVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLEHDFGKVTKQEVKEFFRWAQDHVTEVAHEFYVRKGGANKRPAPDDADKSEPKRACPSVADPSTSDAEGAPVDFADRYQNKCSRHAGMLQMLFPCKTCERMNQNFNICFTHGTRDCSECFPGVSESQPVVRKRTYRKLCAIHHLLGRAPEIACSACDLVNVDLDDCVSEQ

SEQ ID NO: 98 - protein, 621 aa, synthetic construct
MPGFYEVVIKVPSDLDEHLPGISDSFVNWVAEKEWELPPDSDMDQNLIEQAPLTVAEKLQREFLVEWRRVSKFLEAKFFVQFEKGDSYFHLHILIEITGVKSMVVGRYVSQIRDKLIQRIYRGVEPQLPNWFAVTKTRNGAGGGNKVVDECYIPNYLLPKVQPELQWAWTNMEEYISACLNLAERKRLVAQHLTHVSQTQEGDKENLNPNSDAPVIRSKTSARYMELVGWLVDKGITSEKQWIQEDQASYISFNAASNSRSQIKAALDNASKIMSLTKTAPDYLIGQQPVGDITTNRIYKILELNGYDPQYAASVFLGWAQKKFGKRNTIWLFGPATTGKTNIAEAIAHAVPFYGCVNWTNENFPFNDCVDKMVIWWEEGKMTAKVVESAKAILGGSKVRVDQKCKASAQIDPTPVIVTSNTNMCAVIDGNSTTFEHQQPLQDRMFKFELTRRLDHDFGKVTKQEVKDFFRWAADHVTDVAHEFYVTKGGAKKRPAPSDEDISEPKRPRVSFAQPETSDAEAPGDFADRYQNKCSRHAGMLQMLFPCKTCERMNQNSNVCFTHGQKDCGECFPGSESQPVSVVRKTYQKLCILHQLRGAPEIACSACDQLNPDLDDCQFEQ

SEQ ID NO: 99 - protein, 623 aa, synthetic construct
MPGFYEIVLKVPSDLDEHLPGISDSFVNWVAEKEWELPPDSDMDLNLIEQAPLTVAEKLQREFLVEWRRVSKAPEALFFVQFEKGDSYFHLHILVETVGVKSMVVGRYVSQIKEKLVTRIYRGVEPQLPNWFAVTKTRNGAGGGNKVVDDCYIPNYLLPKTQPELQWAWTNMDQYLSACLNLAERKRLVAQHLTHVSQTQEQNKENQNPNSDAPVIRSKTSARYMELVGWLVDRGITSEKQWIQEDQASYISFNAASNSRSQIKAALDNASKFMSLTKTAPDYLVGNNPPEDITSNRIYKILEMNGYDPQYAASVFLGWAQKKFGKRNTIWLFGPATTGKTNIAEAIAHAVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTKRLEHDFGKVTKQEVKDFFRWASDHVTEVSHEFYVRKGGARKRPAPNDADISEPKRACPSVAQPSTSDAEAPVDYADRYQNKCSRHVGMNLMLFPCRQCERMNQNVDICFTHGVMDCAECFPVSESQPVSVVRKRTYQKLCPIHHIMGRAPEVACSACDLANVDLDDCDMEQ

SEQ ID NO: 100 - protein, 623 aa, synthetic construct
MPGFYEIVLKVPSDLDEHLPGISDSFVSWVAEKEWELPPDSDMDLNLIEQAPLTVAEKLQREFLVEWRRVSKAPEALFFVQFEKGDSYFHLHILVETVGVKSMVVGRYVSQIKEKLVTRIYRGVEPQLPNWFAVTKTRNGAGGGNKVVDDCYIPNYLLPKTQPELQWAWTNMDQYISACLNLAERKRLVAQHLTHVSQTQEQNKENQNPNSDAPVIRSKTSARYMELVGWLVDRGITSEKQWIQEDQASYISFNAASNSRSQIKAALDNASKIMSLTKTAPDYLVGQNPPEDISSNRIYRILEMNGYDPQYAASVFLGWAQKKFGKRNTIWLFGPATTGKTNIAEAIAHAVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTKRLEHDFGKVTKQEVKDFFRWASDHVTEVTHEFYVRKGGARKRPAPNDADISEPKRACPSVAQPSTSDAEAPVDYADRYQNKCSRHVGMNLMLFPCRQCERMNQNVDICFTHGVMDCAECFPVSESQPVSVVRKRTYQKLCPIHHIMGRAPEVACSACELANVDLDDCDMEQ

SEQ ID NO: 101 - protein, 624 aa, synthetic construct
MPGFYEIVLKVPSDLDERLPGISNSFVNWVAEKEWDVPPDSDMDPNLIEQAPLTVAEKLQREFLVEWRRVSKAPEALFFVQFEKGETYFHLHVLIETIGVKSMVVGRYVSQIKEKLVTRIYRGVEPQLPNWFAVTKTRNGAGGGNKVVDDCYIPNYLLPKTQPELQWAWTNMDQYLSACLNLAERKRLVAQHLTHVSQTQEQNKENQNPNSDAPVIRSKTSARYMELVGWLVDRGITSEKQWIQEDQASYISFNAASNSRSQIKAALDNASKIMSLTKTAPDYLVGSNPPEDITKNRIYQILELNGYDPQYAASVFLGWAQKKFGKRNTIWLFGPATTGKTNIAEAIAHAVPFYGCVNWTNENFPFNDCVDKMVIWWEEGKMTAKVVESAKAILGGSKVRVDQKCKSSAQIEPTPVIVTSNTNMCAVIDGNSTTFEHQQPLQDRMFEFELTRRLDHDFGKVTKQEVKDFFRWASDHVTDVAHEFYVRKGGAKKRPASNDADVSEPKRECTSLAQPTTSDAEAPADYADRYQNKCSRHVGMNLMLFPCKTCERMNQISNVCFTHGQRDCGECFPGMSESQPVSVVKKKTYQKLCPIHHILGRAPEIACSACDLANVDLDDCVSEQ

SEQ ID NO: 102 - protein, 624 aa, synthetic construct
MPGFYEIVLKVPSDLDEHLPGISNSFVNWVAEKEWELPPDSDMDPNLIEQAPLTVAEKLQREFLVEWRRVSKAPEALFFVQFEKGETYFHLHVLIETIGVKSMVVGRYVSQIKEKLVTRIYRGVEPQLPNWFAVTKTRNGAGGGNKVVDDCYIPNYLLPKTQPELQWAWTNMDQYLSACLNLAERKRLVAQHLTHVSQTQEQNKENQNPNSDAPVIRSKTSARYMELVGWLVDRGITSEKQWIQEDQASYISFNAASNSRSQIKAALDNASKIMSLTKTAPDYLVGSNPPEDITKNRIYQILELNGYDPQYAASVFLGWAQKKFGKRNTIWLFGPATTGKTNIAEAIAHAVPFYGCVNWTNENFPFNDCVDKMVIWWEEGKMTAKVVESAKAILGGSKVRVDQKCKSSAQIEPTPVIVTSNTNMCAVIDGNSTTFEHQQPLQDRMFKFELTRRLDHDFGKVTKQEVKDFFRWASDHVTDVAHEFYVRKGGAKKRPASNDADVSEPKRQCTSLAQPTTSDAEAPADYADRYQNKCSRHVGMNLMLFPCKTCERMNQISNVCFTHGQRDCGECFPGMSESQPVSVVKKKTYQKLCPIHHILGRAPEIACSACDLANVDLDDCVSEQ

SEQ ID NO: 103 - protein, 610 aa, synthetic construct
MATFYEVIVRVPFDVEEHLPGISDSFVDWVTGQIWELPPESDLNLTLVEQPQLTVADRIRRVFLYEWNKFSKQESKFFVQFEKGSEYFHLHTLVETSGISSMVLGRYVSQIRAQLVKVVFQGIEPQINDWVAITKVKKGGANKVVDSGYIPAYLLPKVQPELQWAWTNLDEYKLAALNLEERKRLVAQFLAESSQRSQEAASQREFSADPVIKSKTSQKYMALVNWLVEHGITSEKQWIQENQESYLSFNSTGNSRSQIKAALDNATKIMSLTKSAVDYLVGSSVPEDISKNRIWQIFEMNGYDPAYAGSILYGWCQRSFNKRNTVWLYGPATTGKTNIAEAIAHTVPFYGCVNWTNENFPFNDCVDKMLIWWEEGKMTNKVVESAKAILGGSKVRVDQKCKSSVQIDSTPVIVTSNTNMCVVVDGNSTTFEHQQPLEDRMFKFELTKRLPPDFGKITKQEVKDFFAWAKVNQVPVTHEFKVPRELAGTKGAEKSLKRPLGDVTNTSYKSLEKRARLSFVPETPRSSDVTVDPAPLRPLNWNSRYDCKCDYHAQFDNISNKCDECEYLNRGKNGCICHNVTHCQICHGIPPWEKENLSDFGDFDDANKEQ

SEQ ID NO: 104 - protein, 577 aa, synthetic construct
MLNSNPSVWGDLPRVSANRIDADDAEARSRPIKLSRKQKIMAEVIQRATDGLLLTYNDLVVHLSDLMLMLEGMPGGSKTAEQLLTMIHIKLCAKYNAYEFMLMKTPATQNMNPGAPHYDCQGNLVFKLLNLQGYNPWQVGHWLVMMLSKKTGKRNSTLFYGPASTGKTNLAKAICHAVGLYGCVNHNNKQFPFNDAPNKMILWWEECIMTTDYVEAAKCVLGGTHVRVDVKHKDSRELPQIPVLLSSNHDVYTVVGGNATFGVHAAPLKERITQMNFMKQLPNTFGEITPGMISNWLSHCAHIHQEHLSLEGFAIKWDVQSVGNSFPLQTLCPGHSQNWTFSENGVCWHCGGFIQPTPESDTDSDGDPDPDGAVAGDSDTSANSESTVSFSSNDSGLGSVTSSAPSVPDRAEEIEEIPSECLEWMREEVDRLSAHDINSLAHQATGFILDPIPEEPEERERDLAREDAEPEASTSHTPATKRARVEEGEPWDGTQPITEGDWIDFESRQKRRRLEREEKGGEDEDMEVQESDPSAWGEKLGIVEKPGEEPIVLYCFETLPESDEEGDSDKENKTHTV

SEQ ID NO: 105 - protein, 781 aa, synthetic construct
MAFNPPVIRAFSQPAFTYVFKFPYPQWKEKEWLLHALLAHGTEQSMIQLRNCAPHPDEDIIRDDLLISLEDRHFGAVLCKAVYMATTTLMSHKQRNMFPRCDIIVQSELGEKNLHCHIIVGGEGLSKRNAKSSCAQFYGLILAEIIQRCKSLLATRPFEPEEADIFHTLKKAEREAWGGVTGGNMQILQYRDRRGDLHAQTVDPLRFFKNYLLPKNRCISSYSKPDVCTSPDNWFILAEKTYSHTLINGLPLPEHYRKNYHATLDNEVIPGPQTMAYGGRGPWEHLPEVGDQRLAASSVSTTYKPNKKEKLMLNLLDKCKELNLLVYEDLVANCPELLLMLEGQPGGARLIEQVLGMHHINVCSNFTALTYLFHLHPVTSLDSDNKALQLLLIQGYNPLAVGHALCCVLNKQFGKQNTVCFYGPASTGKTNMAKAIVQGIRLYGCVNHLNKGFVFNDCRQRLVVWWEECLMHQDWVEPAKCILGGTECRIDVKHRDSVLLTQTPVIISTNHDIYAVVGGNSVSHVHAAPLKERVIQLNFMKQLPQTFGEITATEIAALLQWCFNEYDCTLTGFKQKWNLDKIPNSFPLGVLCPTHSQDFTLHENGYCTDCGGYLPHSADNSMYTDRASETSTGDITPSDLGDSDGEDTEPETSQVDYCPPKKRRLTAPASPPNSPASSVSTITFFNTWHAQPRDEDELREYERQASLLQKKRESRKRGEEETLADNSSQEQEPQPDPTQWGERLGFISSGTPNQPPIVLHCFEDLRPSDEDEGEYIGEKRQ

SEQ ID NO: 106 - protein, 668 aa, synthetic construct
MSGNQYTEEVMEGVNWLKKHAENEAFSFVFKCDNVQLNGKDVRWNNYTKPIQNEELTSLIRGAQTAMDQTEEEEMDWESEVDSLAKKQVQTFDALIKKCLFEVFVSKNIEPNECVWFIQHEWGKDQGWHCHVLLHSKNLQQATGKWLRRQMNMYWSRWLVTLCSVNLTPTEKIKLREIAEDSEWVTILTYRHKQTKKDYVKMVHFGNMIAYYFLTKKKIVHMTKESGYFLSTDSGWKFNFMKYQDRQIVSTLYTEQMKPETVETTVTTAQETKRGRIQTKKEVSIKCTLRDLVSKRVTSPEDWMMLQPDSYIEMMAQPGGENLLKNTLEICTLTLARTKTAFELILEKADNTKLTNFDLANSRTCQIFRMHGWNWIKVCHAIACVLNRQGGKRNTVLFHGPASTGKSIIAQAIAQAVGNVGCYNAANVNFPFNDCTNKNLIWIEEAGNFGQQVNQFKAICSGQTIRIDQKGKGSKQIEPTPVIMTTNENITIVRIGCEERPEHTQPIRDRMLNIKLVCKLPGDFGLVDKEEWPLICAWLVKHGYESTMANYTHHWGKVPEWDENWAEPKIQEGINSPGCKDLETQAASNPQSQDQVLTPLTPDVVDLALEPWSTPDTPIAETANQQSNQLGVTHKDVQASPTWSEIEADLRAIFTSEQLEEDFRDDLD

SEQ ID NO: 107 - protein, 672 aa, synthetic construct
MAGNAYSDEVLGVTNWLKDKSSQEVFSFVFKNENVQLNGKDIGWNSYRKELQDDELKSLQRGAETTWDQSEDMEWESAVDDMTKKQVFIFDSLVKKCLFEVLSTKNIAPSNVTWFVQHEWGKDPGWHCHVLIGGKDFSQPQGKWWRRQLNVYWSRWLVTACNVQLTPAERIKLREIAEDSEWVTLLTYKHKHTKKDYTKCVLFGNMIAYYFLSKKKICTSPPRDGGYFLSSDSGWKTNFLKEGERHLVSKLYTDEMKPETVETTVTTAQEAKRGRIQTREEVSIKTTLKELVHKRVTSPEDWMMMQPDSYIEMMAQPGGENLLKNTLEICTLTLARTKTAFDLILEKAETSKLANFSMASTRTCRIFAEHGWNYIKVCHAICCVLNRQGGKRNTVLFHGPASTGKSIIAQAIAQAVGNVGCYNAANVNFPFNDCTNKNLIWVEEAGNFGQQVNQFKAICSGQTIRIDQKGKGSKQIEPTPVIMTTNENITVVRIGCEERPEHTQPIRDRMLNIHLTRTLPGDFGLVDKHEWPLICAWLVKNGYQSTMACYCAKWGKVPDWSEDWAEPKLDTPINSLGSMRSPSLTPRSTPLSQNYALTPLASDLADLALEPWSTPNTPVAGTAASQNTGEAGSTACQGAQRSPTWSEIEADLRACFSQEQLESDFNEELTLD

SEQ ID NO: 108 - protein, 662 aa, synthetic construct
MAAGNTYSEEVLKATNWLQDNAQKEAFSYVFKTQKVNLNGKEIAWNNYNKDTTDAEMINLQRGAETSWDQATDMEWESEIDSLTKRQVLIFDSLVKKCLFEGILQKNLSPSDCYWFIQHEHGQDTGYHCHVLLGGKGLQQAMGKWFRKQLNNLWSRWLIMQCKVPLTPVERIKLRELAEDGEWVSLLTYTHKQTKKQYTKMTHFGNMIAYYFLNKKRKTTEREHGYYLSSDSGFMTNFLKEGERHLVSHLFTEANKPETVETTVTTAQEAKRGRIQTKKEVSIKCTIRDLVNKRCTSIEDWMMTDPDSYIEMMAQTGGENLIKNTLEITTLTLARTKTAYDLILEKAKPSMLPTFNISNTRTCKIFSMHNWNYIKVCHAITCVLNRQGGKRNTILFHGPASTGKSIIAQHIANLVGNVGCYNAANVNFPFNDCTNKNLIWIEEAGNFSNQVNQFKAICSGQTIRIDQKGKGSKQIEPTPVIMTTNEDITKVRIGCEERPEHTQPIRDRMLNINLTRKLPGDFGLLEETEWPLICAWLVKKGYQATMASYMHHWGNVPDWSEKWEEPKMQTPINTPTDSQISTSVKTSPADNNYAATPIQEDLDLALALEPWSEPTTPTFTNLHLTPTPPDSAIRTPSPTWSEIETDIRACFGENCAPTTNLE

SEQ ID NO: 109 - protein, 590 aa, synthetic construct
MAQAQIDEQRRLQDLYVQLKKEINDGEGVAWLFQQKTYTDKDNKPTKATPPLRTTSSDLRLAFDSIEENLTASNEHLTNNEINFCKLTLGKTLLLIDKHVKSHRWDSNKVNLIWQIEKGKTQQFHIHCCLGYFDKNEDPKDVQKSLGWFMKRLNKDLAVIYSNHHCDIQDIKDPEDRAKNLKVWIEDGPTKPYKYFNKQTKQDYNKPVHLRDYTFIYLFNKDKINTDSMDGYFAAGNGGIVDNLTNKERKTLRKMYLDEQSSDIMDANIDWEDGQDAPKVTDQTDSATTKTGTSLIWKSCATKVTSKKEVANPVQQPSKKLYSAQSTLDALFNVGCFTPEDMIIKQSDKYLELSLEPNGPQKINTLLHMNQVKTSTMITAFDCIIKFNEEEDDKPLLATIKDMGLNEQYLKKVLCTILTKQGGKRGCIWFYGPGGTGKTLLASLICKATVNYGMVTTSNPNFPWTDCGNRNIIWAEECGNFGNWVEDFKAITGGGDVKVDTKNKQPQSIKGCVIVTSNTNITKVTVGCVETNAHAEPLKQRMIKIRCMKTINPKTKITPGMLKRWLNTWDRQPIQLSHEMPELYLGKCRW

SEQ ID NO: 110 - protein, 2580 aa, synthetic construct
MSEKTQTFVQNETHVLDMTSDFKSDLSLEKVTSSVEQTDDLVSKIINNNDLDIKDLSFLRNLLLSTLQYLGIAKFVAINITLSILSILMLLINSCAKFTRIVNLSSHILNIITTLGLYFQVSSMEIEEITQTFENEFGTYDDDKILSHYIKICNLPNRKDVYEYISLNDLKYKIKLPDISFYELKNDILSKNKNLHLWIFQKFTDEFLAMWFGVQPYRISNLREMLVISRQGFIPKDLFNEIRKLCNMGVSVIISFIQSKLFDEPFKKRDCTQALKDASVISSPFDTLWNLISKQVCDNSAEERFTQTILDFTSEFDNFLGIPNYKFAKNQKLVNTISKSLDACAKFIRDCPKDKQTEIFPLQGLHTATVKRRNEILTNVMPKFARQEPFVVLFQGPGGIGKTHLVQQLATKCVNSFYQDHEDDYIEISPDDKYWPPLSGQRVAFFDEAGNLNDLTEDLLFRNIKSICSPAYFNCAAADIEHKISPCPFELVFATVNTDLDTLQSKISSTFGQASVFPIWRRCIVVECSWNEKELGPFNYKNPSGHRSDYSHITMNYMSYDDKTQKLALEKEINFDTLFDMIRLRFRKKQQEHDTKISILNNEIQRQSNSKQHFSVCLYGEPGQGKTYNLNKLITTFANATNLKIGSEEKPSIHIFDDYIKDENDENCSKFMDIYNNKLPNNSVIFSATNVYPKTHFFPTFFLTNLIYAFIQPFKQVGLYRRLGFDGYTDIPNSSVNAPIFVQNFKFYERKQHICYFLSLEFLKNIICYIFFFLYFPLKFIKKIDLIEIKDVNKYVYDRYINFLSLSKQIEIVEYPPNLENVEFDFRFNMNKFHRVSFNNPFELDKYIHFNKNSYENLLHFDWKMYLSPRVKHRLALSYEKFFITISEVNKEIIIEELKRYVLLFKQFNIDPNMEINLGEYGSFYYINGKIHLMTINIESNVSEIPVFTDGDYVYISEHKIPVIDLFDNININSKYNLSFDQSIALNSFKTGDSFYSNAKVRKSLSKFVLLNYQTKFKLYLKEAKDKVKNFIETPIGHLLSILLTIFVICYASFKIYSKFSNFFSKDQAIEDQRKGEKKIKKITNYDSDGVQPQRKGEKKIKKVTNYDSDGVQPQSNVKVEEEIKLVFDPTGQKLLFGNDFTSELETLVELEKDDEEFTKSKIDNKSMAGLRREVRRRRYARSKKAQIEKQEVLTLPDVNGFEGGKPYFQIAEEKARKNLCQIYMIANNENCIASKFSDHIVCYGLFVFKKRLASVGHIVEALKCAPGYNLYAGCDQFNGKLYKMNLVRNYRKRELSVWDVDCPNDFVDLTSFFIPKEELYDAENCNTVLGRFGMNKREVYLYGNCEFIQEFFKVDNKGAQEFGYIDWATVDITLTTGGDCGLPYYICERKKFHNKIMGLHFAGNNVNHKTIGMSALIYKEDLVVWKGAERQSKCKFCDVKDIIIAQPDIPKEKYKGYNHEIVWNSLHESSPTTLNEELEHYLNIFPKFTGTIIKHSGDKFYGSVKHSHTQFISKFKTELTVTNGWKLSTAGDCQFESNHISPNTEVMYRVVDVQFNSIFKAFKSQPYIKNFRLIANVYEKDGKQRVTILTIIPVSDFNVKQQTVRQALVPLHLNEDEEVYVTEDVSDIFKTAIKRKQRGILPDVPYETVENETVEILGITHRNMTPEPAQMYKPTPFYKLALKFNLDHKLPVNFNMKDCPQEQKDMMVLDRLGQPNPRITQSLKWAHKDYSPDYELRKYVKEQYMCNIMEYYAGCNLLTEEQILKGYGPNHRLYGALGGMEIDSSIGWTMKELYRVTKKSDVINLDSNGNYSFLNNEAAQYTQELLKISMEQAHNGQRYYTAFNELMKMEKLKPSKNFIPRTFTAQDLNGVLMERWILGEFTARALAWDENCAVGCNPYATFHKFATKFFKFKNFFSCDYKNFDRTIPKCVFEDFRDMLIQANPHMKNEIYACFQTIIDRIQVSGNSILLVHGGMPSGCVPTAPLNSKVNDIMIYTAYVNILRRADRGDITSYRYYRDLVCRLFYGDDVIIAVDDSIADIFNCQTLSEEMKILFGMNMTDGSKSDIIPKFETIETLSFISRFFRPLKHQENFIVGALKKISIQTHFYYATDDTPEHFGQVFKTIQEEAALWEEEYFNKIQSYIQEIIRKFPEISKFFNFESYKSIQKRYIMNGWNEFVKLEKLDLNLNKKKSSKVTGIHSKQYSKFLKFLSRIENEKAALEGNFNKESVNTWYFKMSKAMHLNEIFQKGLISKPLAEFYFNEGQKMWDCNITFRRSKDDLPFTFSGSGTTKACAREQAAEEALVLFSQEDEIVRQINDIQSDCKFCKKMIRYKKLLSGVSIQRQMNVSKITENHVPSAGMMATDPSVAPDSGIATNTQTPSISRVLNPIARALDNPAGTGAPFDKHTYVYNVFTRWPEMSTVVNKSLAAGAEVFKISLDPNKLPKRILQYIQFHKTIIPQIEVQILIGGAAGTVGWLKVGWVPDASTAKKYSLDDLQLVASETINLNSTITMSMIINDSRRNGMFRLTKSDPEPWPGIVCLVEHPITNVQRNDDVNYPVIVSVRLGPDCQLMQPYNDLN

SEQ ID NO: 111 - protein, 1906 aa, synthetic construct
  MOD_RES 600: note = XXXXX
  MOD_RES 1371: note = XXXXX
MNFTKQPASLKYLSSMKLITSQDQDFFNFGEVSREFILEQAYVNGYDFHMLHQDMNCIGFVLSIFDEDARDEYEYKDLNCEYHENLFDAVVDSEFDKIWPHLVLKYITYYPCSLNWRGMPTIPIVYVSKHFWYELYRTGFLNKLYHCGSWTDILLLLSGDVETNPGPVETYKDLCRRKNIRKRKSRIREEIKMQQHIDKIIGQENEEYKIINVNMQGIFSFNEEKEIIKSTAWKFNSTLDKTNSIIDNLIPQLEETLAGFRKTYSKCESKIFGTISVVDVCVDLISALLQVSFAKPAMKIASLAVEVFRLIKKYVSNININIDKIKELLSYGKVALNNNNPIIHVTMQSNSPILEVLLQPNIIVSAIFIALSVVFHKKFTYKKLGIEAMIKRLGDLGRAAKGCSDLNVVLNQAITNHMLEHFGKNVLGLKQEDELKVLVEGYRNWCDEVRDLVGHKINSDGELDSKSIVENIMKDVYEIQRIENMYKKGLEISRNIAELKLPTKLTISFNTHMRYLTEVFKSVDTSGAFGNKPRTQPIVIWLFGESGRGKSGMTWPLAIDLNNSLLDNVDEMRNFSKNIYMRNVEQEFWDNYQGQNIVCXDDFGQMRDSSSNPNPEFMELIRTANIAPYPLHMAHLEDKRKTKFTSKVIIMTSNVFEQDVNSLTFPDAFRRRVDLCAEVKNKDEFTKMCWSKSAGKMVQRLDKGKVKKITGDIHSTVPYIVDLIDPESGEVYKTGLEYEEFLDMCLEKTSQCRDDSAKLNDFLMDYAEKRANRSREIDEVCARTMDEAFVDAYDDVIDVNMQIETVDEMELIEPNKLREMIEQCSNKIVYTYEGIAVKITSLAFKLATLNYEEQWEQIKEMKYYVKVSSGVNYLKRVLSQGMKVCEEWMKEMINYVKEHPWMTVSLILGTLIGILTVVGFWKWLCSGDKKKNPIKRHFINTGNVLILPDRELNTFWKNQESLDLRDMYINRVEEHIISLLKLQHKVVLVPKVTKYILTTVENHAKISDKIILITRNRYLNYQGKFVELICGEINQFFIDPETLDTNVEAFASADLKTFVQRKPIVIEGPEFVEAQTSGDQITLRKQTQKVIEAFASSDAITMARKTPKFVESDDVVEVSMQMWKDQVAQKLITNRVLTNLYKICLVKENGDMVPLLNGLFVRSNIMLAPGHLVGFLSDSDTIEIRNLFDVVFRVPWKDVKKVDVVNAFGESKEAVLLCFPKFVCQHTDLVKHFQDSESMSKFKRCEVTLPVLRYSDKMNRFLATLIECDKVEAYDRPYTLNDSSKGQYILRQGLEYTMPTTNGDCGAPLVINETQVIRKIAGIHVAGDARGKAYAESISQKDLIRAFSKIDVSMQIQLDLDQTLNFNQQQXIIPPNAEFGPEDLDFCDLPSLKMIPVGRLSEPLFEPGKTDIRPSLVYGKISEIKTKPAILRNVIVDGKIVNIKHKNLKKCAMDTPYVSKEMTEEAFQLVKSVWLKGMRNELKKVLTYEEAICGNDSSEFISAINRSSSPGFPWIRDRIKGTKGKQGWFGAEGEYILDEDVFEAVKTRIQNAKNGVRTPVMWVDTLKDERRPIEKVDQLKTRVFSNGPMDFSITFRMYYLGFIAHLMENRITNEVSIGTNVYSQDWNKTVRKLKTMGPKVIAGDFSTFDGSLNVCIMEKFADLANEFYDDGSENALIRHVLLMDVYNSTHICGDSVYMMTHSQPSGNPATTPLNCFINSMGLRMVFELCSKKYSALNGTKCYVMKDFSKHVSIVSYGDDNVINFSDEVSEWFNMETITEAFEKLGFTYTDELKGKNGEVPKWRTIEDVQYLKRKFRYDSKRKVWEAPLCMDTILEMPNWCRGSLDIQEGTKVNCENAIMELSMHEEYVFDKWSKVISKAYQKATGDCLDISTYNGYAQERFLNYYL

SEQ ID NO: 112 - protein, 1771 aa, synthetic construct
MSFQQTNNNATNNINSLEELAAQELIAAQFEGNLDGFFCTFYVQSKPQLLDLESECYCMDDFDCGCDRIKREEELRKLIFLTSDVYGYNFEEWKGLVWKFVQNYCPEHRYGSTFGNGLLIVSPRFFMDHLDWFQQWKLVSSNDECRAFLRKRTQLLMSGDVESNPGPVQSRPVYACDNDPRAIRLEKALQRRDEKISTLIKKLRQEIKNNRIYTQGFFDDLKGAKGEVGQLNGNLTRICDFLENSLPTLTAQIQTTVLTTTDKYVNLKEDLLKVAILLVLVRLLMVWKKYRAALIVIILFVMHFYGFDKQILDIVLDLKDKILQTTTQAGTETLEEVVYHPWFDTCGKLIFAVLAFFAIKKIPGKQDWDNYISRLDRIPKAIEGSKKIVDYCSEYFNLSVDEVKKVVLGKELKGTQGLYDEIHVWAKEIRHYLDLDERNKITLDTETAAKVEDLYKRGLKYSEEKIPDRDIARFITTMLFPAKSLYEQVLLSPVKGGGPKMRPITVWLTGESGIGKTQMIYPLCIDILREMGIVKPDAYKHQAYARQVETEYWDGYNGQKIVIYDDAFQLKDDKTKPNPEIFEVIRTCNTFPQHLHMAALQDKNMYSQAEVLLYTTNQFQVQLESITFPDAFYNRMKTHAYRVQIKQEKSIWVRNARGEEYNALDVTKLNKDEAIDLSVYEFQKMRFDDESATKWIDDGEPISYDEFARTICKAWKEEKEKTFHQLQWLEAYASRTVAQGGSETSEYYDVWDETYFSNLLSQGFMAGKSLIEMEAEFASDAETFNAYIEYKKNIPKETKWSKWMTILDEQISALSTKIRELKNKAYKFISEHPYLTALGFIGVMISAFAMYSFFERTLTDDTITSEVGSSGDNKTQKISKRVVEVGGSGDVKTTKPAKTAVEVGSSGDSKTMKNKITKVEVGSSGDSKTQKQRNTKVEVGKELEKEAETQGCSDPAAHALVLDVLQKNTYCLYYERMVKGEMKRYRLATATFLRGWVCMMPYHFIETLYARKVAPSTNIYFSQPNCDDVIVVPVSHFIAPNAERVELTTACTRIHYKDETPRDCVLVNLHRRMCHPHRDILKHFVKKSDQGNLRGVFQGTLATFHQSANELCRAYQWLQAIRPLDQEITIYHEDTDMFDYESESYTQRDCYEYNAPTQTGNCGSIVGLYNKRMERKLIGMHIPGNVSECHGYACPLTQEAIMDGLNRLEKLDPVNNITVQCCFEPPSDIKDTMSGETPEGKFCAIGKSNIKVGQAVKTTLLKSCIYGMLSKPITKPAHLTRTRLPNGEIVDPLMKGLKKCGVDTAVLDAEIVESAALDVKQVVLTQYNSMLDVNKYRRFLTYEEATQGTGDDDFMKGIARQTSPGYRYFQMPRKLPGKQDWMGSGEQYDFTSQRAQELRRDVEELIDNCAKGIIKDVVFVDTLKDERRPIEKVDAGKTRVFSAGPQHFVVAFRKYFLPFAAYLMNNRIDNEIAVGTNVYSTDWERIAKRLKKHGNKVIAGDFGNFDGSLVAQFFGQSCGKSFYPWFKTFNDVNTEDGKRNLMICIGLWTHIVHSVHSYGDNVYMWTHSQPSGNPFTVIINCLYNSMIMRIVWILLARKLAPEMQSMKKFRENVSMISYGDDNCLNISDRVVEWFNQITISEQMKEIKHEYTDEGKTGDMVKFPSLSEIHFLKKRFVFSHQLQRTVAPLQKDVIYEMLNWTRNTIDPNEILMMNINTAFREIVYHGKSEYQKLRSGIEDLAMKGILPQQPQILTFKAYLWDATMLADEVYDF

SEQ ID NO: 113 - protein, 1759 aa, synthetic construct
MESDKSMACLNRILMNKMMFVEDKISTLKMVADYYQKEVKYDFDAVESPREAPVFRCTCRFLGYTIMTQGIGKKNPKQEAARQMLLLLSGDVETNPGPVQSRPVYYRYNDPRYTRLEKAIERRDDKIKTLIKELRRQIKNRKIYSQGMFDKLTKQISDGIKDGVGSEQMNGNLTRICDFLENTLPGLQANIQATVIDTTDKYVSLKEDIMKIVLVILLVRLLMVWKKYRASLCVILIFIFKFYGFDQKLIDLIMDLKNKIFSQGALEDTVEEVVYHPWFHTCGKIIFAVMAFLTIKKIPGKQDWDSYITRLDRIPKSIEGAKKITDYCSEYFNIANDQIKMMVLGKTKEELQRANGLYGEIQAWAQEVRQYLELDQRNKIDLDTETANRVEQLWIKGLKFKSEPLLSKEMSALVHTTLLPAKQLYEYVSCSPVKGGGPRMRPICLWLVGESGVGKTEMVYPLCIDVLREMGMIKKDDFHHQVYGRQVETEFWDGYKGQKIVIYDDAFQKKDDKTAANPEIFEVIRSCNTFPQHLHMAALHDKNTFSAAELLLYTTNDYNVKLESITFPDAFFNRMGDMAYKVSPKKEYGIETEKGNSGKTYLKLDKSKLDKTKAIDLSVYEFQKIVRDEKSDAGWIDSGSPLDYEDFAKLVCSKWKEAKQSSMNKLKFLEEYAIRAQVGSEENSEYGDCIDFVDDIAKRLQKGETLEEIEFDYASDPEMFTQYYHFKSTIKPASRWQKYKDRMDICLSDCKTYLAKKYEEIKKILAEHPILTILGMIGVALSALAMYYWFSKSLDPVEAEVAPSGDAKTVRLPRKLVEIGASGDVKTQKIVKPVVETEWHRNNKGEIEISCDECGMHRMSAFNNMTDEEFDNCTYEDLNKDQKRELAQWSTKDSWLGRFFLSRDRKNKVGIWAEVGQSGDVKTNKAQIKRVEAGAEELVTVALTQGCSDDAAHNLMIDVFQKNTYRMSYFRGDKRYQLGNCTFVRGWSFIMPYHFVQAVFARRLPPNTIISLSQQMSEDLMQIPLSHFFSAGVDNFYLTDNCVRLPFKNGDFRDCVMVNLHSRMCTPHRDLVRHFILTSDQGKLKGSFSGAMATFHVNNMGLYRVYNWLNAVRPCDKKIEIFHPEDGFEYPEESYIQRDCYEYNAPTRTGDCGSIIGLYNKYLERKIIGMHIAGNDAEEHGYACPLTQECLETAFSALVNKNKKNISSQFYYEIPNMVDPLGDSSVPEGKFYALGKSSIRVGQAVNSSIIPSRIYGKLSVPTMKPALLKPTILNNKVHNPLLSGLKKCGVDTAVLSDDEVLSASQDVCRVMLNQYNKNLNKTKYQRILTYEEAIRGTQDDEFMCAINRTTSPGFPYAQMKRNAPGKQQWMGFGEEFDFTSNYALALRKDVEQLIEDCASGKISNVIFVDTLKDERRDIAKVNVGKTRVFSAGPQHFVVAFRQYFLPFAAWLMHNRISNEVAVGTNVYSSDWERIAKRLKTKGSHVIAGDFGNFDGSLVAQILWAIFWEIFVVWLKQFIDIENSEGKRILCICLGLWSHLVHSVHIYEDNVYMWTHSQPSGNPFTVIINCLYNSIIMRLSWIRVMEKFQPRLKSMKWFNEYVALITYGDDNVLNIDAKVVEWFNQINISEVMTEMRHEYTDEAKTGDIVKSRKLEDIFFLKRKFRFSPELQRHVAPLKIEVIYEMLNWSRRSIDPDEILMSNIETAFREVVYHGKEEYDKLRSAVLALKVPQELPENPQILTYNQYLHDIEYLADPLYDF

SEQ ID NO: 114 - protein, 2987 aa, synthetic construct
MMTTQTNQLFNRSLNDELGNRTDTITLHPWNVSEYVTSTGLAEAEFEACTGKDLEIQKYLVTVSEECPCGHPTTFSEIDMSRGMVMASSDNIYSDDIVTLVPTENGYEDSDPCFCENQKEDCDNCMYLQILRMAPGKWNMTYGLRHNHQAQLTYYLTEERGFTCEGYTLEADTSDSEGFGDTADYTLYCIQYSGKYKQRRQWHVLLDDESCGFCKYRVTTGLIVPEVPRHWTNELGRRQNLLVPKVLGQYVDFIEAKSPLRLDWLSMSKLVSGKCSLPNFYVNLTTLRTFQVAGGKFLPYLYNGSAANNDLKLPIQVTAQGDEDTPAGELSIEQDTHENTTLAESTDASTAYVATEEFSMMPWITDGPHTYPDLTERWTKAFQFQWTTSQAQGEIIQRFDLPIEAIQNFINSPNALPWRQHAFYKSDIELKVQVNSQPGQSGYLILGAMYEASEGTAIGNRVDHAANIVAMPHMRISAGSSNSGDMVIPFIRHFPVGCILNNAFDVPQYFVTLFVAPLLQLRTGADGPQVVDVTIMIRFPNCEFYGQRTTEQIVTAQGWAPDLTQDGDVESNPGPFLSGLLGTVAAVGKTVAGAGSSIGSIATGVSGVANGIGSIGSAAGKVIGGVESLLRPLFPKKDMDRPQNIIEPTNFYLQQNTSLSLATGTNNVKLLQLQAENSVSHPPGFVPVDDQFNNRFILSVFGLSDYFQWFSGSASGTLLYSFDVTPLKSFTRGIPLQPEFYLTPMAALSGQYGGYHGDMEMRLTFAVSKFHSGRVFIVYSPDVVPTFDNIGAYYNVLLDVQDQSVYTFKIPYQVPTPYAPIFESLQGDTGTFLTPAVSAGNVRCMAYGFVSIFVENQLRVMQTAAPTIDVLVELRGADNFHLVLPAGGKFRSLSTITTTEAPVVTAMGDERREPHTVNPPPRRIMPVWAAQLNESYDCRDVVKRYHDWFDIVSPAVVAGRGFTDMYMNVTVFHVPVPAFDAAPIQNVQFTDMLVGSSILNKSMVLARERINLATNNTISIRVPWTNYASMISNSINPASGRSNTMAPYSNGRVVVYIEYLSSYTTTLGAFRVVWDVTAGSASTRPDTYTPDMLTLLHDGFRFAKGDFNYQMDFTPVPSGANTAIRMRCFRAYGDGGNLYVFQGFPKMLGTYTPRQAISGVTPTRGGLQRQNIIGGGQRDLTQDGDIESNPGPTQSKPTGAQPPPDDILTDEDREGLEGGSIRISFFEKLGDYCKRALGWSGERIVEFIRELRSIKKHVGSIPGMTRISELITTIKELSVVTNGLNRMSAAVEELNEQIKKTREKVETFVGGVGGKLNSLDTGDLVSNGIEYVAYILNIYNSKSVGMTLINVAALLSKMGLGRYLVNDLVDRLGTFAKGEDTEEIEREYTSLIITGVLGAFGLGTMNVEKEGFVKPFLSNVKDFFRNGFAVKKFLDSHFKCINDICGWVRSKIFGKVDKGGLTVDLLVWCERVQVLAEVYNYDTILNDPEFAETLLSLQDEAFEFDRLFIASRIRPPNQYSMYRTKLQRAIDLLGQQGTMQKSKPVPFCLWVYGHSGCGKSHVCDNVLTEIGSALGINTANPIYTRSPDVEFWNGYTGQKLISWQDFAKITTGETYRKQVSELSSLIEPTPFNPPFAALEDKRKIADAWAVYVSSNKAFPEVQNMGMEDSALFYRRRHALVKMRINPEIIQEYARREPPISLEDKYKGQTVYYPSNIPSEDFATRGPYFHVQFAFHVTSLSTAEPTEWLGYDGFIAEVTRRAIDHRQREVDACIQRRGIYSKLRRSAPTGVEFREEVRTLKEELDRLTQERVAEAHGSDNAVKNLATTMDYEKRATFEETEALGSVILQYRLDPNCDCTNQAIMKAATSNEPRKICEVSFCEKCEPVNQRKKNVYKAMSEQLEGVAVSNPFGGIAMIERGDSARIPRAGRQYTPLDYARQATIGTLKWIQGKGLPAGYAYWSEIMPVCCHKKEILELAYFENVGDNIELVLDLSSMKARHGVSFPLFHKLEWFDLRKLDAWATTGFKVILPVVCEQSKFNWNNCIEVKPGERGRIPITPKWAPELIGRVTLEEFKRSWSCDAVQKPSVSYYKIEKDLIRFLVSGTYVQIEGVENMTAGLNHLMSLHTKDTVAFCNSFFYWKSGGKLRAAKFYPFVNGMLKDMSFIFSNIMIAVGAVMLAYKVYRLIRNSSECVEAQAKDYDQKTEAPKIPKVNFVQSTPVVVAAKGDEELSIIHNSLCQLRKGSMRLLGVRLCSNFVLAPQHLAWVPGEFGISLHSSGAWSTELIFEATSVKYSCVKGFDYAVYRFVTLPAGRNIVNYFTTRAQGATLKSEATLMTLSGASLQLRPVRISQYTGALTYDNASFPGTGTSMIGWQYWLGAQSVRCGSLIMSNNLLCGFHVAQNLKTGDAYAVSICKEMLVEALRILGATPFDMKMKRTTPITTVGEPKMQPPETAVVIPLGKAIEPVRYSGKSNLEKSIMHGELAEPFRIPAAQSATAKGECIGYDIVMKGCEKQFKPPKPIDPEEVEKIGDYLIERLVPQSIPLIPTISEPLSLGEAIAGIDGIPLMCGMKLNTSIGWPLCNEYPKGTKKSNIIRVDREEGEVHVDVVAFDDYAKANTLRKQAILPPTTFMDFPKDELLKPGKDTRLINGAPLHHTLDMRRYLMEFFAAITTINNKIAVGIDVHSGDWALIHGGADDVVDEDYSGFGPGFHSQWLTVVCPIAVAWCKHHKKVDKEYEDVVNCLIMELQNAYHVAGDLVYQVLCGSPSGAFATDRINSLANLCYHCLCHLRKYGTLTGFWSHYTLVYGDDTRRRETAYTGDEFQECMASVGIVVNRDKSGVTSFLKRQFIPIDHRDVRVMLAPLPRPIVEDILNWVRKPYASKLSALEETVGSYLSEIFHHGQDEFNNSRSKIQAILARHGSHPELPTFDDLFRQKYLSNGVWPVLMPLANALGPIPAAESEPTHKVTSGQVVCVPHEAGECCAITLGG

SEQ ID NO: 115 - protein, 2210 aa, synthetic construct
MAPVVSRDRHRHKIPKPHQPAPPHRCTVWCPEDCGWYVGRCSCPKSCQREGWDDFFVADKVKPPSYVASKTSVADVVDWLLEEDPATDGPSEFDLTQFFQAYTDKSHQIHRDYAPDQLAQALDMAYILSVDPPDIKLPEYEATRFTHDTSYKGKLPRWLRVYGFKSRELAKKAITNIKGGAHWAKGLFKQAWDTLPGWSEVEAYFKAFFAGIITGVEDALSKSPSSVWTSLKLTPLLYIWRNINECSDIPVILGAFWATLELYNIPSKVYDLVSTALGPMVQDLARRVINIIKGDGNGPKQEGGRPSFSIPGVLLATFLSAIILGSMPSDGIIKKVLRGCATAAGLVGGFNAVKSIITTVQGASACKDVKKLASQLMCVTTMAATVSTRGERQVLASMLNDLNESVRERLVDPAFAALVPQLSAMSSKIMELSTINAAALSAARKRVPAKIVVLCGPPGHGKSVAAHKLAKMLNPNEPSIWNPFSDHHDEYTAEDVVIIDETPAEPGQWVEDLIAMGSNSPFVPNYDRVENKTRCFDSKYVLITTNHNPLINPTHSRAAALARRLTWVYVNSPDVADFLRQHPGVAPPATLFKADCSHLNFDIHPYNSIGTTAVVGHNGTTPLPRAKRTSLEGLCKHIKDLPDREGPPDGVPERMVLVAPDKGTARFVEAVINTYHNSGLVAQPAAWDTTPQKYQLAVTWQGSTSSVVGQRWDCNPQTPFVAPHFTRNMFKRVLGTEVPEYHLLAYACRITSSSLGDKSLPVPNPTVVINDPSPTRLALALMRHLKNPIASGLRVVWDLFRGCATGPKRLFTWALSQEWNPMPVTTAFTFPAGTVILHTAGGVRVVVLPPGPQFGLTEVTHLADHSGQDDPVVPDMFGQTWTELLWRLLKVIGTFLANYGVAIAGLTLSIAAFKTANKSTRNDRQGWLSGSGVALSDEEYDEWMKYSKKKGKKINADEFLQLRHRAAMGNDDDDARDYRSFYTAYQLGREGNNCEDLPLHPAVGPTTGGGYYVHIGNGVGITLKHVASGEDVIKELGNDLVKIRARHHKMGDPAMVVGEGAPVKFVTGHLVVDARNESVVFDQTRLSVVRVKVPGLETQRGYCGLPYVNSAGHVVGLHQGSYGVGDKVFTPITDAPAASPDTIMWRGLECTRSDIVTHLPHGTKYSISPGMREEAHKCTHQPASLGRNDPRCNQTQVAMVVKALTPYTSAPAIEKLDPCMVAAITEVRTAIQSLTPKGGFRPLTFAAAWQSLDLSTSAGALAPGKTKRDLCDPDTGMPAGKYREMLLAAWSRAGTGTPLDHTYIVALKDELRPVEKVAEGKRRLIWGADARVALIASAALTPVANALKTVTNLLPIQVGVDPSSANCVSSWVGRLQRHDHCLELDYSKWDSTMSPVIINIAIDILCNTCGSDSLRMAVAQTLKSRPTALVEGVSVPTKSGLPSGMPFTSQINSIVHWILWSATVRKCSLPLHIGSVNELAPFLTYGDDGLYTIPSHLTKSIDEIISTLKGYGLSPTAPDKGANVEIKRTSFTYLSGPVFLKRRIVLTPGGHRALLDLTSLARQPVWVNGPRRSVWNHEAQPIEIDAETRTIQLQNVLIELAWHQPQDFDHVLALVVKSAEASGLTIPRYSQEEARAIYDGRYYGIQHVSLPNNSDLIREGNMSDNKSTPEQQHESSRAMDAGATGAAAAAPAPPVAAAPASGLVGALVAEPQSGPSAEQWRTAYTLFGTVSWNANAGPGTILTVGRLGPGMNPYTQHIAAMYGGWAGGMDIRITIAGSGFIGGTLAVAAIPPGVDPESVNVLRMPHVLIDARGGVPLEVTLEDIRTSLYHPMGDTNTASLVIAVMTGLINPLGTDTLSVTVQLETRPGRDWVFFSLLPPTAGVASADPSQLLTRVALATSPEVRFGTGVLGILGLPSNPSVNRVYDVQSRTRGWSFPIPSSSVFMGDARNVEHNRRVMVQSSAPNNPLSDVFPDGFPDFVPQSDTEPDGGAVIAGQVLPHPGDNDNFWRLTPVVRGNTTAAINTIPERFNQVYFINLADEEAVSAATEELRFNGIQGIFGQRTNARAVQVMQGYVPRAEHIIRPAGFAGVGPQGPNVPIGFAGTMPNFNATASGADDLVPVWGPTLVHTASLLAGTTYELAENSMYVFSVSTSTSTFELGMLANGTWLGPAQLAGTGITWTEVLSVTYMGMRFAYNPLSGQGIGGESRRL

SEQ ID NO: 116 - protein, 2210 aa, synthetic construct
MAPVVSRDQCKPKTPKPHRPAPPHRCTTRCPEDCGWYVGRCSCPNVCQREGWDDFFVADKVKPPSYVASKTSVADVVDWLLEEDPATDGPSEFDLTQFFQAYTDKSHQIHRDYAPDQLAQALDMAYILSVDPPDIKLPEYEATRFTHDTSYKGKLPKWLRVYGIKSRELAKKAVTNIRGGAHWAKGLFKQMWDSLPGWSEVEAYFKAFFAGIITGVEDALSKSPSSVWTSLKLTPLLYIWRNINECSDIAVILGAFWATLELYNIPSKVYDLVSTALGPMVQELARKVINVVKGDGSGPKQEGGRPSFSIPGVLLATFLSAIILGSMPSDGLIKKILRGCATAAGLVGGFNAVKSIITTVQGASACKDVKKLASQLMCVTTMAATVSTRGERQVLASMLNDLNESVRERLVDPAYASLVPQLSAMSNKIVELSTMNASALSAARKRTPAKIIVLCGPPGHGKSVAAHKLAKMLNPNEPSIWNPFSDHHDEYTAEEVMVIDETPAEPGQWIEDLIAMGSNSPFVPNYDRVENKTRCFDSKYVIITTNHNPLINPTHTRAAALARRLTLVYVNSPDVADFLRQHPGVPPPATLFKADCSHLHFDIHPYNSIGTTAIVGHNGTTPVPRAKRVSLEGLCKHVKEMPDREGPPDGVPERMVLVAPDKGTARFVEAVINTYHNSGLVAQPAAWDTTPQPYQLAVTWQGSNSTVTGQRWDCNPQTPFVAPHFTRNMFKRVLGTEVPEYHLLAYACRITSSSLGDKSLPVPNPTVVINDPSPTRLALALMRHLKNPIASGLRVVWDLFRGCATGPKRLFTWALSQEWNPMPVTTAFTFPAGTVILHTAGGVRVVVLPPGPQFGLTEVARLADHSGQDDPVVPDMFGATWTELLWRLLKVIGTFLANYGVAIAGLTLSIAAFKTANKSAKNDRQGWLSGSGVALSDEEYDEWMKYSKKKGKKINADEFLQLRHRAAMGNDDDDARDYRSFYTAYQLGREGNNCDDIPLHPAVGPTTGGGYYVHIGNGVGVTLKHVASGEDVIKELGNDLVKIRTKHHKVGDPAMVVGDGMPVKFVTGHLVVDTRSESVVFDQTRLNVIRVKVPGLETRRGYCGLPYVNSAGQVVGLHQGSYGVGDKVITPITPEPTAPPDTIMWRGLECARSDIVTHLPHGTKYSVSPGMKEEATKCSHQPAPLGRNDPRCGQTQVAMVVKALSPYTGSPAVEKLDGCLVAAISEVRTAIQSLTPKGGFRPLTFAAAWQSLDLSTSAGALAPGKTKRDLCDPDTGMPTGKYKEELLRAWSRAGTGTALDHTYIVALKDELRPVEKVAEGKRRLIWGADARVALIASAALSPIANALKTVTNLLPVQVGVDPSSASCVSAWVNRLNRHDHCLELDYSKWDSTMSPVLINIAIDILCNTCASDGLRVAVCQTLKTRPTALVEGVAVPTKSGLPSGMPFTSQINSIVHWILWSATVRKCSLPLNIGSVNELAPFLTYGDDGLYTIPSHLTKSIDEIVSTLKGYGLSPTAPDKGMNIEIKKTSFTYMSGPVFLKRRIVLTPGGHRALLDLTSLARQPVWVNGPRRSVWDHEAQPIEIDSEVRTIQLQNVLIESAWHQPQDFNQVAALVYKSAEASGITIPRYSLEEARAIYDGRFYGIQHVSMPCNSDLIREGNMSDNKSIPEQQHESSRAMDAGATGAAAAAPAPPVAAAPASGLVGALVAEPQSGPSTEQWRTAYTLFGTVSWNANAGPGTILTVGRLGPGMNPYTQHIAAMYGGWAGGMDIRITIAGSGFIGGTLAVAAIPPGVDPESVNVLRMPHVLIDARGGVPLEVTLEDIRTSLYHPMGDANTASLVIAVMTGLINPLGTDTLSVTVQLETRPGRDWVFFSLLPPTAGVASADPSQLLTRVALATSPEVRFGTGVLGILGLPSNPSVNRVYDVQSRTRGWSFPIPSSSVFMGDARNVEHTRRVMVQSSAPNNPLSDVFPDGFPDFIPQSDTEPDGGAVIAGQVLPHPGDNDNFWRLTPVVRGNTTAAINTIPERFNQVYFINLADEEAVSAATEELRFNGIQGIFGQRTTARAVQVMQGYVPRAEHIIRPAGFAGVGPQGPNVPIGFAGTMPNFNATASGADDLVPVWGPTLVHTASLLAGTTYELAENSMYVFSVSTSTSTFELGMLANGTWLGPAQLAGTGITWTEVLSVTYMGMRFAYNPLSGQGIGGESRRL

SEQ ID NO: 117 - protein, 1929 aa, synthetic construct
MASAIALSSSTAQNKITLKSVASRLQQTDDPDIRVWSQSVGFHLQFSNWKCANAFCRFVTDAYNLTPYKECARSITRQLTSLSNYLSAQTGVSVSGTQFLLSPSDVEVPVAKTGESVSDIMVPSYSVNGTSMEFDSMAQLAQALTTGFTFSVNDAQIGNAPAQTGESVSGTGFIAEACPSCALYDKCPNCTSELINDDGSSQSPGDIPHWTHHKIASGIVNILSSDMSSMEDDDFANIAAHVKKALGTNSHPANNDMSKDQLNWLLNIAEASLIRKADRTALPMNAARIAARRGWREKLFNEPADKLYTLLRKSKDSFQKSAIWGILFEKASNAKHYTEIVFQDIVKLIKEECNPSNNFYFKVMAQSFLDHFRMLVIDNPDPVANLPKFILKLKPLNLKMIIENHENTAEGWIVTLTAVAELYGWLEFAVDLVPKIVSELYDLLTSATQKCFSMVRELLTNLNILKAESFDFTNPFWYALAALLSYFVTGFLPNNAKCSAIKQTLNGATTLVAGITAIQKLAAMFSAWSNESVVDDLSTKVIGLTEADNPTVTQDIDAVTNLQIMAEQLKDQIKLKTLDPTFQPYLPVLRNLMSTTDSVISHCAKRKALATQRTAPVCIILTGPAGCGKTTLAYAIANRLSAQKPSVLNLNIDHHDAYTGNEVCIIDEFDSNPDSKFVEFVVEMVNTNPMLLNCDLIENKGKTFSSKYVIMTSNNETPVKPNSTRAPPFYRRVRIIDVTNPGVMSFKYENPGQEVPSYLFSNDFNHLSMSMRGFGAFSKTRVIDPEGRKTCGLEGPPGQRVDVDDIVRYMQRMYRENQMNFKSEAGNNRLKTPRFAFVTQRKHVDTVYKILAAAKTTYNGYYSLTKDSFDVNEGHNIGSSVFVVGDDKEIPHNCKIFRCNHLAMFRHPELAHIEGDNFRAALGVTMSDQDVTLMFYHIRGKHIQDEVRLDELPANHHIVTVHSVYDMAWALNRHLSLTGKWQALKAVYDLYMTPDILPAALRHWMDNTKFSSDHVVTQFIVPGGTIILETCNGARMWATSRRLIRAGGISNNNGPEGGFRFGSIAPRDIPWSEILREFLNLISLIWSRVKGATIVLTALLLYMKRYKPRSEAKGKTKGGRGAIRHGGKGIVLSDDEYDEWREFNMEKRMDMSVDEFLMLKHRAALGSDDTGAIQFRSWWTARQMRESTGLDHDDVTVIGKGGVRHEVHRTEIMKAPKQKKKSFAWGEDMYAEGDGKIVNHVNAIVPVTGLCGEHIGYAVHIGHGKCISLKHVLKTGSYVFNQKPIDVTFDGELAHFQIQQPPSSAAPVTFSSKPTRDPWGRSVSTEWKHDTYNTTAGKMYGSICWTATRTQPGDCGLPYVDRAGQVVGLHAGSGGDSAPGRKIVIPVTKFKLPSNTVLSNRFWKEEAPTISYKGLTVQETGVNKAVLKGTNYHVSPAHVDDYQDCTHQPANLGAQDERYPVSLTSIVINNLEPYKQPTQGPPTEVLNKAYNMLVQHYEPLIPKATTHLEMGDAFAALNVKTSCGPYITGRKKDHIDPETGKWDETLRNHINARWSLATQGVPIPHEYQLGLKDELRPKDKIAVGKRRLIWGCDVGVAVVAASAFKEVSSAIMAMSEFDFIQVGINMDGTAVETLYKRLYTPGTHRYCVDYSKWDSTQPPNVTRMSLELLRHFTDKSPVVDSAVATLSSPSIAVFGGVSFKTNGGLPSGMPLTSILNSLNHCLLVGSAIIQVLESKGVDVNWNIYDTIDLFTYGDDGVYIVPNFVHSVMPEVFSCLSSYGLKPTRTDKSSAPITEIPLSEPIEFLKRQFVRNQFGVRALLDRSSLIRQFYYIKGKNTMEWTKPPEQIDLTSRTAQLQVVMLYASQHGREFYKKCLDYYQLAMEYEGIKLDAPTYDEALAKYNANFNGVEDCDLLPAGYDEHRLDKIVFEN

SEQ ID NO: 118 - protein, 1763 aa, synthetic construct
MSQTLSFVLKTHSVRKDFVHSVKLTLARRRDLQYIYNKLSRTIRAEACPSCASYDVCPNCTSGDVPDDGSSTMSIPSWEDVTKSSTYSLLLSEDTSDELCPEDLVNVAAHIRKALSTQSHPANAEMCKEQLTFLLVMAEAMLPQRSRASIPLHQQHTAARLEWREKFFSKPLDFLLERVGVSKDILQTTAIWKIILEKACYCKSYGEQWFTAAKQKLREMKNFESDTLKPLIGGFIDGLRFLTVDNPNPMGFLPKLIGLVKPLNLAMIIDNHENTISGWIITLTAIMELYNITECTIDIITSVITAFYDKIGKATKFYSCVKALFTGFRSEDVANSFWYMAAAILCYLITGLIPNNGRFSKIKACLAGATTLVSGIVATQKLAAMFATWNSESIVNELSARTVALSELNNPTTTSDTDSVERLLELAKILHEEIKIHTLNPIMQSYNPILRNLMSTLDGVITSCNKRKAIARKRQVPVCYILTGPPGCGKTTAAQALAKKLSDQEPSVINLDVDHHDTYTGNEVCIIDEFDSSDKVDYANFVIGMVNSAPMVLNCDMLENKGKLFTSKYIIMTSNSETPVKPSSKRAGAFYRRVTIIDVTNPFVESHKRARPGTSVPRSCYKKNFSHLSLAKRGAECWCKEYVLDPKGLQHQSMKAPPPTFLNIDSLAQTMKQDFLLKNMAFEAEDGCAEHRYGFVCQQEEVETVRRLLNAVRARMNATFTVCVGPETSHSIGCTAHVLTPNETFNGKKFVVSRCNEASLSALEGNCVKSALGVCMSDKDLTHLCHFIKGKIVNDSVRLDELPANQHVVTVNSVFDLAWAVRRHLTLAGQFQAIRAAYDVLTVPDKIPAMLRHWMDETSFSDDHVVTQFVTPGGIVILESCGGARIWALGRNVIRAGGVTATPTGGCVRLMGLSAPTMPWSEIFRELFSLLGRIWSSVKVSALVLTALGMYASRFRPKSEAKGKTKLKIGTYRGRGVALTDDEYDEWREHNASRKLDLSVEDFLMLRHRAALGADDNDAVKFRSWWNSRTKMANDYEDVTVIGKGGVKHEKIRTNTLKAVDRGYDVSFAEESGPGTKFHKNAIGSVTDVCGEHKGYCIHMGHGVYASVAHVVKGDSFFLGERIFDLKTNGEFCCFRSTKILPSAAPFFSGKPTRDPWGSPVATEWKPKMYTTTSGKILGCFATTSTETHPGDCGLPYIDDNGRVTGLHTGSGGPKTPSAKLVVPYVHIDMKTKSVTAQKYDVTKPDISYKGLICKQLDEIRIIPKGTRLHVSPAHTEDYQECSHQPASLGSGDPRCPKSLTAIVVDSLKPYCENVEGPPHDVLHRVQKMLIDHLSGFVPMNISSETSMLSAFHKLNHDTSCGPYLGGRKKDHMANGEPDKQLLDLLSAKWKLATQGIALPHEYTIGLKDELRPVEKVSEGKRRMIWGCDVGVATVCAAAFKGVSDAITANHQYGPIQVGINMDSPSVEALFQRIKSAAKVFAVDYSKWDSTQSPRVSAASIDILRYFSDRSPIVDSASNTLKSPPVAIFNGVAVKVSSGLPSGMPLTSVINSLNHCLYVGCAILQSLEAKAIPVTWNLFSTFDIMTYGDDGVYMFPIMYASISDQIFGNLSSYGLKPTRVDKSVGAIEPIDPDSVVFLKRTITRTPQGIRGLLDRSSIIRQFYYIKGENSDDWKSPPKHIDPTSRGQQLWNACLYASQHGLEFFNKVYRLAERAVEYEELHFEPPTYASALDHYNSQFNGVEARSDQIDSSGMTALHCDVFEV

SEQ ID NO: 119 - protein, 1763 aa, synthetic construct
MSQTLSFVLKTHSVRKDFVHSVKRTLQRRRDLQYLYNKLSRPIRAEACPSCASYDVCPNCTSGSIPDDGSSKGQIPSWEDVTKTSTYSLLLSEDTSDELHPDDLVNVAAHIRKALSTQSHPANVDMCKEQLTSLLVMAEAMLPQRSRSTLPLHQKYVAARLEWREKFFSKPLDFLLEKIGTSRDILQITAVWKIIIEKACYCKSYGEHWFEAAKQKLREIKSYEHNTLKPLIGAFIDGLRLMTIDNPNPMGFLPKLIGLIKPLNLAMIIDNHENTLSGWVITLTAIMELYNITECTIDVITSIITGFYDKIGKATKFYSQIKALFTGFRSEDVANSFWYMAAAILCYLITGLIPNNGRLSKIKACLAGATTLVSGIVATQKLAAMFATWNSESIVNELSARTVAISELNNPTTTSDTDSVERLLELAKILHEEIKIHTLNPIMQSYNPILRNLMSTLDGVITSCNKRKAIAKKRPVPVCYILTGPPGCGKTTAALALAKKLSDQEPSVINLDVDHHDTYTGNEVCIVDEFDSSDKVDYANFVIGMVNSAPMVLNCDMLENKGKLFTSKYIIMTSNSETPVKPSSRRAGAFYRRVTIIDVANPLAESHKRARPGTSVPRSCYKKNFSHLSLAKRGAECWCKEYVLDPKGLQHQSIKAPPPTFLNIDSLAQTMKQDFTLKNMAFEAENGHSEHRYGFVCQQGEVETVRRLLNAVRTRLNATFTVCVGSEASSSIGCTAHVLTPDEPFNGKKYVVSRCNEASLSALEGNCVQSALGVCMSTKDLTHLCHFIRGKIVNDSVRLDELPANQHVVTVNSVFDLAWALRRHLTLAGQFQAIRAAYDVLTAPDKVPAMLRHWMDETSFSDEHVVTQFVTPGGIVILESCGGARIWALGHNVIRAGGVTATPTGGCIRFMGLSAQTMPWSEIFRELFSLLGRIWSSIKVSTLVLTALGMYASRFRPKSEAKGKTKSKVGPYRGRGVALTDDEYDEWREHNATRKLDLSVEDFLMLRHRAALGADDADAVKFRSWWNSRSRLADDYEDVTVIGKGGVKHEKIRTNTLRAVDRGYDVSFAEESGPGTKFHKNAIGSVTDVCGEHKGYCVHMGHGVYATVAHVAKGDSFFLGERIFDLKTNGEFCCFRSTKILPSAAPFFPGKPTRDPWGSPVATEWKPKPYTTTSGKIVGCFATTSTETHPGDCGLPYIDDNGRVTGLHTGSGGPKTPSAKLVVPYVHIDMKTKSVTAQKYDVTKPDISYKGLICKQLDEIRIIPKGTRLHVSPAHTEDFEECSHQPASLGSGDPRCPKSLTAIVVDSLKPYCDKVEGPPHDILHRVQKMLIDHLSGFVPVNISSETSMLSAFHKLNHDTSCGPYLGGRKKDHMTNGEPDKPLLDLLSAKWKLATQGIALPHEYTIGLKDELRPVEKVAEGKRRMIWGCDVGVATVCAAAFKGVSDAITANHQYGPVQVGINMDSPSVEALHQRIKSAAKVYAVDYSKWDSTQSPRVSAASIDILRYFSDRSPIVDSAANTLKSPPIAIFNGVAVKVSSGLPSGMPLTSVINSLNHCLYVGCAILQSLEARGVPVTWNLFSTFDMMTYGDDGVYMFPMMFASVSDQIFANLSAYGLKPTRVDKSVGSIEPIDPESVVFLKRTITRTPQGIRGLLDRSSIIRQFYYIKGENSDDWKTPPKSIDPTSRGQQLWNACLYASQHGVEFYNKIYKLAQKAVEYEELHLEPPTYHSALEHYNNQFNGVEARSDQIDSSGMTALHCDVFEV

SEQ ID NO: 120 - protein, 1879 aa, synthetic construct
MAQTLSKISNKENASVGLWPKRFKPHQPTPTWMVRCGPLDHDSRHGRDPVRASPQAKRVRTPNPYPRHLKPAASAVVRSGTNPSHLKPTSTDVVRSGPETPCCEAKDGGVVRSCKTCNLKPAHDSKAVSFFPAQTDGLTGDEPEFIAEACPSCVLYDTCPNCTSRAINDDGSTDGTIPSWDQIETTPAFLSLLSNTDEEMSADELTNLAAHLRKAFETGSHPPNVDYSKDQLQGLLEMAEAALPPARRQTLPFYQQRLEARRTWREKIFNLPLDELSKILTTSKDRFQRCAAWKVVLEKAVLAKEYGEEAYAYAQEALKNINSFDVNLVLKMAAGTFIGHLRMMTVDNPDMVSYLPKLIVKLKPLTLKMIIDNHENTKEGWLVTLTSLAELYGMVEVAIDFVPTVIGNLFDLLMKTTSKVYSMFKSVILATFTSESLDFTNPFWYAIAAILCFLITGAIPHNGKMKIIKNILSNATGIVAGVKAIQTLGAMFSTWSNERLVNDLSSRTIAITELNNPTITADIDAVINLQRLAETLREEVKSHTLNPLMQPYTPILRNLMSALDNVISCCTRRKAIATKRTAPVAVILTGPPGCGKTTAAFALAKRLSQQKPSIISLDVDHHDTYTGNEVCIIDEFDSSDKVDYANFVVNMVNTNPMVLNCDLVENKGKTFRSKYVIMKSNSETPVKPTSRRAGAFYRRVMIVDVKNTAVENWKRENPGKPVPKWCFNKDFSHLHLSMRGTEAYWREYVLDPTGRNHQSQKAPPDQHVTLEQLDQKMVVQHTTNTSEFVTQAGEVPVFGFVCQNNEIDTVYNLLAAVKARYGANFNLYKGMTRTAHENSGCGAHVHVISREDNFRGKAFTVNRSRLESVPHLEGDSFRRSLGVVMSDKDVTTMFYYIKGKVINDQVNLTELPANQHVVTVHTVYDMAWALRRHLKWTGQWQLIKAAYEIMCYPDTAACALRNWMDSTDFSEEHVVTQFIAPGGTIILESCYGARMWATGQRLIRAGGLTEAGGPQGGVRFAGLGARNVPWSEILREFMTLISHIWSQIKGATVVLTALTFYLKRFRPRVEAKGKNKNKGPRKNTGVALTDDEYDEWRQYKAEKKLDLTVEDFLQLRHRAAMGADDTDAVKFRCWYSERQRNYHDLEDVTIIGRGGVKRELIRKGPLRPRGNDFYDEPDDWYSEGVIDGVTHKNAIVSVDDVDGMHKGYALHIGHGVYMSLKHVVSGNAKILSEEPKNLTFNGELATFRLNTTLPTAAPVGTSKPIKDPWGNPVSTDWQFKNYNTTSGNIYGACGSSCSLTRQGDCGLPYVDDHGVVVGLHAGSGGDKCPSRKLIVPYVKVDMRIRDTCTKEYYKDNVPMISYKGLLVKETGEPRTIMKGTRLHVSPAHTDDYEECTHQPASLGAGDPRCPMSLTGIMVNNLQPYTEAPRTDTATLNRVTKMLISHMEGYVPKIHKTEEDMISAFYMLNHDTSCGPYIGGRKKDHVKDGVLDKNLLDLLSSKWNRAKCGLALPHEYALGLKDELRPKDKVAVGKRRLIWGCDVGVSTVCRAAFKRVSESIMANHALGFIQVGINMDGPAVEDPFKRLERPKHDRYCVDYSKWDSTQPPKVTSQSIDILRHFTDKSPIVDSACATLKSNPIGIFNGVAFKVAGGLPSGMPLTSIINSLNHCLMVGSAVVKALEDSGVQVTWNIFDSMDLFTYGDDGVYIVPPLISSVMPKVFSNLRQFGLKPTRTDKTDAEITPIPADEPVEFLKRTIVRTENGVRALLDKSSIIRQFYYIKAENTENWTVPPKKIDTSSRGQQLYNAGLYASQHGEEFYTNKIIPLVQRAIEFEGLHIEVPEFHQAVQAYNGYFNGTEDQPSQIALASGGTGFGGEVFEN

SEQ ID NO: 121 - protein, 1214 aa, synthetic construct
NKGKTFTSKYVIMTSNTETPVKPTSRRAGAFYRRVMIVDVTNNAVEKWKSDNPGKAVPKWCFNKDFSHLSLSLRGTEAYCKEYVLDPTGRNHQSRRAPPPQQVTLEQLAQKMVVQHTTNTSEFVTQAGDVPVFGFVCQNNEIDTVYNLLAAVKARYGANFNLYKGMVRTAHENSGCGAHVHVISREDNFRGKVFTVSRSRLESVPHLEGDSFRRSLGVVMSDKDVTTMFYYIKGKVINDQVNLTELPANQHVVTVHTVYDMAWALRRHLKWSGQWQLIKAAFEIMCYPDAAACALRNWMDSTDFSEEHVVTQFIAPGGTIILESCHGARMWATGKRLIRAGGITEAGGPQGGVRFAGLGARNVPWSEILREFMTLISHIWSQIKGATVVLTALTFYLKRFRPRIEAKGKNKNKGARKNTGVALTDDEYDEWRQYRTEKNLDLTVEDFLQLRHRAAMGADDSDAVKFRCWYSERQRNYHDLEDVTIIGRGGVKRELIRKGPLRPRGNDYYDEPDDWYSEGVIDGVAHKNAIVSVDDVDGMHKGYAIHIGHGVYISLKHVVSGNARILSEEPKNLTFTGELATFRLNNILPTAVPVGTSKPIKDPWGNPVSTDWQFKNYNTTSGNIYGACGSSCSLTRQGDCGLPYVDDHGVVVGLHAGSGGDKCPSRKLIVPYAKVDMRVRDTCTKQCYKDNSPTISYKGLLVKETGEPRTIMKGTRLHVSPAHTDDYEECTHQPASLGAGDPRCPISLTGIMVNNLQPYTEASPGPDTATLNRVSKMLTSHMEGYVQKSQTEEGYGSAFYMLNHDTSCGPYIGGRKKDHVKDGVLDKNLLDLLSSKWNRAKLGLALPHEYALGLKDELRPKDKVAVGKRRLIWGCDVGVSTVCRAAFKRVSESIMANHALGFIQVGINMDGPAVEDLFKRLERPKHDRYCVDYSKWDSTQPPKVTSQSIDILRHFTDKSPIVDSACATLKSNPIGIFNGVAFKVAGGLPSGMPLTSIINSLNHCLMVGSAVVKALEDSGVQVSWNIFDSMDLFTYGDDGVYIVPPLISSVMPNVFANLKQFGLKPTRTDKSDAEITPIPADEPVEFLKRTIVRTENGIRALLDRSSIIRQFYYIKAENTENWTIPPKKIDTPSRGQQLYNACLYASQHGEEFYTNKIIPLVQRAIEFEGLHIEVPEFHHAVAAYNGYFNGTEGQPNQIAFASGGLGLSSEVFEN

SEQ ID NO: 122 - protein, 1881 aa, synthetic construct
MAQTLSKISNKENASSGLRPKRFKPHQPIPTWMVRCEPLDHDSRRGRDPVRASPQAKRVRTPTPYPRHLKPAASAVVRSGNNPSHLKPASTDVVRSGPQPLCCEAKDGGVVRSCKTYNLKPAHESKAVAFSLPKTDGPTGNEPEFIAEACPSCALYDTCPNCTSKVINDDGSTDGTIPSWDQIETTPAFLSLLSNTDEEMSADELTNLAAHLRKAFETGSHPANVDYSKDQLQGLLEMAEAAVPPARRQTLPFYQQRLEARRTWREKIFNQPLEEINKILTTSKDRFQRCAAWKVILEKAVLAKEYGEEAYAYAQQALKNINSFDVNLVLKMAAATFIDHIRMMTVDNPDLVSYIPKLIVKLKPLTLKMIIDNHENTKEGWLVTLTSLAELYGMVEVAIDFVPTVVGKLFDLLMKTTSKMYSMFKSVVLATFTSESLDFTNPFWYAIAAILCFLITGAIPHNGKMKIHKNILSNATGIVAGIKAIQALAAMFSTWSNERLVNDLSSRTIALTELNNPTITADIDAVINLQRLAEVLRDEVKSHTLNPLMQPYNPILRNLMSALDKVISCCTRRKAIATKRTAPVAVILTGPPGCGKTTAAFALAKRLSQQKPSIISLDVDHHDTYTGNEVCIIDEFDSSDKVDYANFVVNMVNTNPMVLNCDLIENKGKTFTSKYVIMTSNTETPVKPTSRRAGAFYRRVMIVDVTNNAVDKWKSDNPGKAVPKWCFNKDFSHLSLSLRGTEPYSKEYVLDPTGRNHQSRRAPPPQQITLEQLAQKMVVQHTTNTSEFVTQAGDVPVFGFVCQNNEIDTVYNLLAAVKARYGANFNLYKGMVRTAHENSGCGAHVHVISREDNFRGKAFTVNRSRLESVPHLEGDSFRRSLGVVMSDKDVTTMFYYIKGKVINDQVSLTELPANQHVVTVHTVYDMAWALRRHLKWSGQWQLIKAAYEIMCYPDTAACALRNWMDSTDFSEEHVVTQFIAPGGTIILESCYGARMWATGQRLIRAGGLTEAGGPQGGVRFAGLGARNVPWSEILREFMTLISHIWSQIKGATVVLTALTFYLKRFRPRVEAKGKNKNKGPRKNTGVALTDDEYNDWKQSKAEKNLDLTVKDFLQLRHRAAMGADNTDAVKFRYWYSKKQKIYHDLENFPIIGRGGLKRELIRKGPLRPRGNDFYDEPDDWYSEGVIDGVTHKNAIVSVDDVDGMHKGYAIHIGHGVYISLKHVLTGNARILSEEPKGITISGELATFRLNNILPTAVPVGTNKPIKDPWGNPVSTDWQFKNYNTTSGNIYGACGSSCSLTRQGDCGLPYVDDHGVVVGLHAGSGGDKCPSRKLIVPYVKVDMRIRDTCTKESPTKTHKPTFSYRGLLGKETGEPRTIMKGTRLHVSPAHVDDYEECTHQPASLGAGDPRCPISLTGIMVNNLQPYTEASPGPDTATLNRVSKMLTSHMEGYVPKVHKTEEDSISAFYMLNHDTLCGPYIGARKKDHVKDGVLDKNLLDLLSSKWNRAKLGLALPHEYALGLKDELRPKDKVAVGKRRLIWGCDVGVSTVCAAAFKRVSESIMANHALGFIQVGINMDGPAVEDLFKRLERPKHDRYCVDYSKWDSTQPPKVTSQSIDILRHFTDKSPIVDSACATLKSNPIGIFNGVAFKVAGGLPSGMPLTSIINSLNHCLMVGSAVVKALEDSGVRVTWNIFDSMDLFTYGDDGVYIVPPLISSVMPKVFANLRQFGLKPTRTDKSDAEITPIPADEPVEFLKRTIVRTENGVRALLDRSSIIRQFYYIKAENTENWTVPPKRIDTPSRGQQLYNACLYASQHGEEFYTSKIVPLIERAVKLEGLHIEVPEFHQAVAAYNGYFNGTEGQPNQIAHASGGLGLSGEVFEN

SEQ ID NO: 123 - protein, 2254 aa, synthetic construct
MANCRPLPIGQLPNRIFDTPRLTPGWVWACTSEATFKLEWLQDPVVIRPPDVFVAQGVVDDFFRPKRVLQGDPQLIAQVLLGDANGPLVGPVSMQQLTSLLHEVSQALSDHKHPLANRYTRASLQRYADTLSNYIPLVDILTGPKDLTPRDVLEQLAAGREWECVPDSALKKVFRDMWQYICEGCDSVYIKLQDVKRKMPHIDTTVLKQFIITLTDTISMATALDTKTWLAHILGWLKPTCLVMIMQQHVNSPQGWAATLTALAELYYGIMPLTETLGSVASWVTDKFADMATSTWGKFKSWWDSLYTPQAGNDLIILGGVVGLVYFMVFGDAPTQMFTKKLMRVCGFITSTVAAIKAAMWIVDYFKQREHEHQVRITLARWAALQEVIKQNRCAGLSEVTKLKECCEVLLNEVTELMYKLGASPLAGLIRSTSDVIQTTINELAQLMAYDTQRKPPAMVVFGGPPGIGKTRLVEALAKQLGEVSHFTMTVDHYDTYTGNTVAIWDEFDVDSKQAFIEATIGIVNCAPYPLNCDRPEAKGRVFTSKYVLATTNCPTPVMPDHPRAMAFWRRITFIDVTAPTIEQWLVDNPGRKAPTSLFKDDFSHLQCSVRGYTAYDEKGNTLSGKVARARYVSVNNLLDLIKEKYNSEAADVKHLWFTVPQAIHKQARDIILGWLRFHSYPNTVADNIPLSEVRDPTCFGYVVISDVDPPRHVAEHVAHIEVESILRTDIVGLLREGGGGLFRALKVKSAPRNCIINKVMMQAHHTTLQVLTSQEPHPPNLPRPRRLVFVESPIDIISALRHHVGFCTIPGIVKLITSGVGLGVENLGNFLQSIAGNVRFPLQSECSLLRTPSGDVLFYTSGQAAVWATPARFPIVTPGEASVGKEVCSESSWWDILKALFSTLVVAFGPIATLVLTAHNLAYLNTRENTLSEAKGKNKRGRGARRAIALRDDEYDEWQDIIRDWRKEMTVQQFLDLKERALSGASDPDSQRYNAWLELRAKRLSAGAYQHAVVDIIGKSGHRREVIRTQVMRAPREPKGDTYDSEGRGYVVPMTAQEKHTGWAVHIGNGRLVTCTHVANMCDRVAEVEFKVAETDRDTCIITAPLGHLPSVALGNGPPAFYTTNFHPIRVLDEGSWDTTTTRVTGWRVVINNGTTTAPGDCGQPYLNARRQLVGVHAATSTCGVKKLVSRVQTKKTAKATFPWKGLPVTTMPDAGGLPTGTRYHRSIAWPKLLPEETHAPAPYGVNDPRHPFSQHQMIANNLQPYINTPVALDQTLLQRAVKHTKGYLDQIIGTHRSPNLTYAAAVESMAHDTACGPNLPGRKKDYMTDQGEPIGPLKQMLEEAWDMAHRGVPRRHEYKLALKDELRPIEKNDQGKKRLLWGCDAGVSMIANAVFKPVTERLVDTVPMHPVAVGICMDSPQIEQMNQALTGRVLYCLDYSKWDSTQNPAVTCASVDILASYAEDTPLSSAAIATLCSPAVGRLDDIGLTVTSGLPSGMPFTSVINSVNHMIYFAMAVLEAYEEFKVPYMGNIFDNETIYTYGDDCVYGLTPATASIMPVVVKNLTSYGLVPTAADKSQSIEPTDTPVFLKRTFSQTPFGLRALLDETSLARQCYWVKANRTTDLFEPAAVDVDIRKNQLEVMLAYASQHPRSVFDKLAGMAEVTASAEGYQLVNVNWANAVATYNAWYGGTDGGRAPTNEDEEPEVFVMEAPAPTRSVASNPEGTQNSNESRPVQPAGPMPVAAAQALEMAVATGQINDTIPSVVRETFSTYTNVTWTTRQPTGTLLARMSLGPGLNPYTLHLSAMWAGWGGSFEIKVIISGSGMYAGKLLCALIPPGVDPSAVDQPGAFPHALVDARITDGVTFTLGDVRAVDYHETGVGGAIASLALYVYQPLINPFETAVSAAMVTIETRPGPDFGFTLLKPPNQAMEVGFDPRSLLPRTARTLRGNRFGRPITAVVIVGVAQQINRHFSAEGTTLGWSTAPIGPCVARVNGKHTDNTGRAVFQLGPLSNGPLYPNIINHYPDVAASTIFNTGTAVNDNTTGGGGPMVIFNDVGDVVEDVAYQMRFIASHATSQSPTLIDQINATSMAVCSFGNSRADLNQNQLNVGIELTYTCGNTAINGIVTSFMDRQYTFGPQGPNNIMLWVESVLGTHTGNNTVYSSQPDTVSAALQGQPFNIPDGYMAVWNVNADSADFQIGLRRDGYFVTNGAIGTRMVISEDTTFSFNGMYTLTTPLIGPSGTSGRSIHSSR

SEQ ID NO: 124 - protein, 2278 aa, synthetic construct
MASKPFYPIEFNPSVELQVLRSAHLRVGGREQMFETINDLNDHVRGVVAKLWCKHLHRSLAAAPTFTEEGLLDSFLSKPPVDINPDTTFRELFGINPHEQFPLSIHDLAKLQGELVDAARNPGHVLRRHYSTDSLTALINKITKFVPVHATLQEMQARRAFERERAELFRELPHADLDVSRQQKSYFYAMWRQVVKKSKEFFIPLVKCTSWRKKFTEPAEIVRQVLVHFCEGMRSQFSTNANYINLSLIAKLRPTVLTMILQQHKNTYRGWLATVTALVEVYSNLFQDMRDTAVSAVSAITLVFETIKDFVVNVIDLVKSTFQSQGPTSCGWAAIIAGALLILMKLSGCSNTTSYWHRLLKVCGGVTTIAAAARAVVWVRDIIAEADGKARLKKYMARTAALLELAASRDVTGTDELKRLLDCFTQLIEEGTELIQEFGTSPLAGLTRSYVSELESTANSIRSTILLDTPRKTPVAIILTGPPGIGKTRLAQHLAAGFGKVSNFSVTLDHHDSYTGNEVAIWDEFDVDTQGKFVETMIGVVNTAPYPLNCDRVENKGKVFTSDYIICTSNYPTSVLPDNPRAGAFYRRVTTIDVSSPTIEDWKKKNPGKKPPPDLYKNDFTHLRLSVRPFLGYNPEGDTLDGVRVKPVLTSVDGLSRLMETKFKEQGNEHRNLWITCPRDLVAPAASGLKAYMAANRALAQVFQEPSSQDIGETCTSRVYVSCNNPPPTYSGRVVKITAINPWDASLANSMLSMFETTSHIPASIQREIMYRVWDPLVHLQTREPNTQMLPYINRVVPVSSAFDFIRGLRHHLGLCSVKGMWRAYQGWNSSSSILEFLSKHMADVAFPHNPECTVFRAPDGDVIFYTFGSYACFVSPARVPFVGEPPKNVHSNITRNMTWAETLRLLAETITESLVHFGPFLLMMHNVSYLATRSGREEEAKGKTKHGRGAKHARRGGVSLSDDEYDEWRDLVRDWRQDMTVGEFVELRERYALGMDSEDVQRYRAWLELRAMRMGAGAYQHATIIGRGGVQDTIIRTQPMRAPRAPRNQGYDEEAPTPIVTFTSGGDHIGYGCHMGNGVVVTVTHVASASDQVEGQDFAIRKTEGETTWVNTNLGHLPHYQIGDGAPVYYSARLHPVTTLAEGTYETPNITVQGYHLRIINGYPTKRGDCGTPYFDSCRRLVGLHAATSTNGETKLAQRVTKTSKVENAFAWKGLPVVRGPDCGGMPTGTRYHRSPAWPNPVEGETHAPAPFGSGDERYKFSQVEMLVNGLKPYSEPTPGIPPALLQRAATHTRTYLETIIGTHRSPNLSFSEACSLLEKSTSCGPFVAGQKGDYWDEDKQCYTGVLAEHLAKAWDAANRGVAPQNAYKLALKDELRPIEKNAQGKRRLLWGCDAGATLVATAAFKGVATRLQAVAPMTPVSVGINMDSYQVEVLNESLKGGVLYCLDYSKWDSTQHPAVTAASLGILERLSEATPITTSAVELLSSPARGHLNDIVFITKSGLPSGMPFTSVINSLNHMTYFAAAVLKAYEQHGAPYTGNVFQVETVHTYGDDCLYSVCPATASIFQTVLANLTSFGLKPTAADKSETIAPTHTPVFLKRTLTCTPRGVRGLLDITSIKRQFLWIKANRTVDINSPPAYDRDARGIQLENALAYASQHGHAVFEEVAELARHTAKAEGLVLTNVNYDQALATYESWFIGGTGLVQGSPSEETTKLVFEMEGLGQPQPQGGEKTSPQPVTPQDTIGPTAALLLPTQIETPNASAQRLELAMATGAVTSNVPNCIRECFASVTTIPWTTRQAANTFLGAIHLGPRINPYTAHLSAMFAGWGGGFQVRVTISGSGLFAGRAVTAILPPGVNPASVQNPGVFPHAFIDARTTEPILINLPDIRPVDFHRVDGDDATASVGLWVAQPLINPFQTGPVSTCWLSFETRPGPDFDFCLLKAPEQQMDNGISPASLLPRRLGRSRGNRMGGRIVGLVVVAAAEQVNHHFDARSTTLGWSTLPVEPIAGDISWYGDAGNKSIRGLVSAQGKGIIFPNIVNHWTDVALSSKTSNTTTIPTDTSTLGNLPGASGPLVTFADNGDVNESSAQNAILTAANQNFTSFSPTFDAAGIWVWMPWATDRPGASDSNIYISPTWVNGNPSHPIHEKCTNMIGTNFQFGGTGTNNIMLWQEQHFTSWPGAAEVYCSQLESTAEIFQNNIVNIPMNQMAVFNVETAGNSFQIAILPNGYCVTNAPVGTHQLLDYETSFKFVGLFPQSTSLQGPHGNSGRAVRFLE

SEQ ID NO: 125 - protein, 2208 aa, synthetic construct
MFGTKAPDSVQEGTLFRELFGVDQTEQFPLSLADLARLQGELVDATRTPGHALRQKYTMTTIQDLINKITKVVPVQATLNEMHARRQFERERADLFHELPLVDEDAVAQPKTYFYTMWRQVVKKGKAYFCPLVKTSAWRTKISAITEPIKDFLIAFWQAVQQEMGVNPQYLQLAWLQKLKPTTLTIILQQHKHTVSGWLATMTALVEVYSNLFDDLRKSSVTIVSSIGAFFDICKDFVSQVVELVKTTFTAQGPTDLGWAAVLAGAAMILLKMSGCPGVIGMWTKVLKICGGITTITAAARGVRWLKDLYEEAEGRRLPKMYMARGAALIELAASREVTGVDELKGLLDCFTILIEEGTELIHKFGTSPLAGLVRTYVSELETQANNIRSTIKLDTPRRVPVVIILTGAPGIGKTRLAQYIGQRFGKTSNFSVAVDHHDGYTGNTVCIWDEFDVDSKGAFVETMIGIANTAPFPLNCDRVENKGRVFTSDYVICTSNYPTSVIPDNPRAAAFYRRVLTVDVSAPDLEEWKKRNPGKRPTPDLYQDDFSHLKLMLRPYLGYNPDGDTLEGPRVAPTQISIAGLITLMERRFKEQAGPLQNLWLQVPKTLVEQSTNMVKAFMYANRAVCDVIPNPATRDITETALSKVFVCGTAPPPEFVGKHIVITGIEVGDASIANSLLSMFTTTTRLSAAAQREYMYRVWSPLIHIQDRSMNTQNLPYINRVIPVTSHWDFLRGLRHHLGFTSIPGMWKAFQGWRTSQGIVDFVAHHMADVTFPSNPECTIFRTPDADVVFYTFGSYVCFATPARVPYVGTPPTTIHSNTPRCMTWGETLALLCEVVAEFVLHFGPVILSAANIAYLMTRGSRTEEAKGKTKHGRGMRHGHRAGVSLSDDEYDEWRDLMRDWRRDMSVNDFLMLRERSALGMDDEDVARYRAWLEIRAMRMAGGAYTHATIIGRGGVRDEIIRTSPRRAPTRPQQHYEEEGPTAIVEFTQGGDHIGYGVHIGNGNVITVTHVASTSDEVNGSAFKITRTVGETTWVQGPFSQLPHMQIGSGSPVYFTTRLHPVFTISEGTFETPNITVNGFHVRIMNGYPTKKGDCGLPYFNSNRQLVALHAGTDTQGETKVAQRVVKEVTTQDEFQWKGLPVVKSGLDVGGMPTGTRYHRSPAWPEEQPGETHAPAPFGSGDKRYTFSQTEMLVNGLKPYTEPTAGVPPQLLSRAVTHVRSYIETIIGTHRSPVLTYHQACELLERTTSCGPFVQGLKGDYWDEEQQQYTGVLANHLEQAWDKANKGIAPRNAYKLALKDELRPIEKNKAGKRRLLWGCDAATTLIATAAFKAVATRLQVVTPMTPVAVGINMDSVQMQVMNDSLKGGVLYCLDYSKWDSTQNPAVTAASLAILERFAEPHPIVSCAIEALSSPAEGYVNDIKFVTRGGLPSGMPFTSVVNSINHMIYVAAAILQAYESHNVPYTGNVFQVETIHTYGDDCMYSVCPATASIFHTVLANLTSYGLKPTAADKSDAIKPTNTPVFLKRTFTQTPHGIRALLDITSITRQFYWLKANRTSDPSSPPAFDRQARSAQLENALAYASQHGPVMFDTVRQIAIKTAQGEGLVLVNTNYDQALATYNAWFIGGTVPDPVGHTEGTHKIVFEMEGNGSNPEPKQSNNPMVVDPPGTTGPTTSHVVVANPEQPNGAAQRLELAVATGAIQSNVPEAIRNCFAVFRTFAWNDRMPTGTFLGSISLHPNINPYTSHLSGMWAGWGGSFEVRLSISGSGVFAGRIIASVIPPGVDPSSIRDPGVLPHAFVDARITEPVSFMIPDVRAVDYHRMDGAEPTCSLGFWVYQPLLNPFSTTAVSTCWVSVETKPGGDFDFCLLRPPGQQMENGVSPEGLLPRRLGYSRGNRVGGLVVGMILVAEHKQVNRHFNSNSVTFGWSTAPVNPMAAEIVTNQAHSTSRHAWLSIGAQNKGPLFPGIPNHFPDSCASTVVGAMDTSLGGRPSTGVCGPAISFQNNGDVYENDTPSVMFATYDPLTSGTGVALTNSINPASLALVRISNNDFDTSGFANDKNVVVQMSWEMYTGTNQIRGQVTPMSGTNYTFTSTGANTLVLWQERMLSYDGHQAILYSSQLERTAEYFQNDIVNIPENSMAVFNVETNSASFQIGIRPDGYMVTGGSIGVNVPLEPETRFQYVGILPLSAALSGPSGNMGRARRVFQ

SEQ ID NO: 126 - protein, 2280 aa, synthetic construct
MVSKPFKPIVLNATFEWQVFKRCYLRVAPREAFCENLSELHHYFARRVNAWLKHATRTLPDGYTFVEEGLLDMFGTKAPDSVQEGTLFRELFGVDQTEQFPLSLADLAKLQGELVDATRTPGHALRQKYTMTTIQDLINKITKVVPVQATLTEMHARRQFERERADLFHELPLVDEDAVSQPKTYFYTMWRQVVKKGKAYFCPLVKTSAWRTKISAITEPIKDFLIAFCQAVQQEMGVNPQYLQLAWLQKLKPTTLTIILQQHKYTVSGWLATMTALVEVYSNLFDDLRKSSVAIVSSIGAFFDICKDFVSQVVELVKTTFTAQGPTDLGWAAVLAGAAMILLKMSGCPGVIGMWTKVLKICGGITTITAAARGVRWLKDLYEEAEGRRLAKMYMARGAALIELAASREVTGIDELKGLLDCFTILIEEGTELIHKFGTSPLAGLVRTYVSELETQANNIRSTIKLDTPRRVPVVIILTGAPGIGKTRLAQYVGQRFGKTSNFSVAVDHHDGYTGNTVCIWDEFDVDSKGAFVETMIGIANTAPFPLNCDRVENKGRVFTSDYVICTSNYPTSVIPDNPRAAAFYRRVLTVDVSAPDLEEWKKRNPGKRPTPDLYQDDFSHLKLMLRPYLGYNPDGDTLEGPRVAPTQISIAGLITLMERRFKEQAGPLQNLWLQVPKTLVEQSTNMVKAFMYANRAVCDVIPNPATRDIAETALTKIFVCGTAPPPEFVGKHIVITGIEVGDASIANSLLSMFTTTTRLSAAAQREYMYRVWSPLIHIQDRSINTQNLPYINRVIPVTSHWDFLRGLRHHLGFTSIPGMWKAFQGWRTSQGIVDFVAHHMADVTFPSNPECTIFRTPDADVVFYTFGSYVCFATPARVPYVGTPPTTIHSNTPRCMTWGETIALLCEVVAEFVLHFGPVILSAANIAYLMTRGSRTEEAKGKTKHGRGMRHGHRAGVSLSDDEYDEWRDLMRDWRRDMSVNDFLMLRERSALGVDDEDEARYRAWLEIRAMRMAGGAYTHATIIGRGGVRDEIIRTAPRRAPTRPQQHYEEEAPTAIVEFTQGGDHIGYGVHIGNGNVITVTHVASTSDEVNGSAFKITRTVGETTWVQGPFSQLPHMQIGSGSPVYFTTRLHPVFTISEGTFETPNITVNGFHVRIMNGYPTKKGDCGLPYFNSNRQLVALHAGTDTQGETKVAQRVVKEVTTQDEFQWKGLPVVKSGLDVGGMPTGTRYHRSPAWPEEQPGETHAPAPFGAGDKRYTFSQTEMLVNGLKPYTEPTAGVPPQLLSRAVTHVRSYIETIIGTHRSPVLTYHQACELLERTTSCGPFVQGLKGDYWDEEQQQYTGVLANHLEQAWDKANKGIAPRNAYKLALKDELRPIEKNKAGKRRLLWGCDAATTLIATAAFKAVATRLQVVTPMTPVAVGINMDSVQMQVMNDSLKGGVLYCLDYSKWDSTQNPAVTAASLAILERFAEPHPIVSCAIEALSSPAEGYVNDIKFVTRGGLPSGMPFTSVVNSINHMIYVAAAILQAYESHNVPYTGNVFQVETVHTYGDDCMYSVCPATASIFHAVLANLTSYGLKPTAADKSDAIKPTNTPVFLKRTFTQTPHGVRALLDITSITRQFYWLKANRTSDPSSPPAFDRQARSAQLENALAYASQHGPVVFDTVRQIAIKTAQGEGLVLVNTNYDQALATYNAWFIGGTVPDPVGHTEGTHKIVFEMEGNGSNPEPKQSNNPMVVDPPGTTGPTTSHVVVANPEQPNGAAQRLELAVATGAIQSNVPEAIRNCFAVFRTFAWNDRMPTGTFLGSISLHPNINPYTAHLSGMWAGWGGSFEVRLSISGSGVFAGRIIASVIPPGVDPSSIRDPGVLPHAFVDARITEPVSFMIPDVRAVDYHRMDGAEPTCSLGFWVYQPLLNPFSTTAVSTCWVSVETKPGGDFDFCLLRPPGQQMENGVSPEGLLPRRLGYSRGNRVGGLVVGMVLVAEHKQVNRHFNSNSVTFGWSTAPVNPMAAEIVTNQAHSTSRHAWLSIGAQNKGPLFPGIPNHFPDSCASTIVGAMDTSLGGRPSTGVCGPAISFQNNGDVYENDTPSVMFATYDPLTSGTGVALTNSINPASLALVRISNNDFDTSGFANDKNVVVQMSWEMYTGTNQIRGQVTPMSGTNYTFTSTGANTLVLWQERMLSYDGHQAILYSSQLERTAEYFQNDIVNIPENSMAVFNVETNSASFQIGIRPDGYMVTGGSIGINVPLEPETRFQYVGILPLSAALSGPSGNMGRARRVFQ

SEQ ID NO: 127 - protein, 2334 aa, synthetic construct
MAVASRPCGVATSVLPAKKPLSFFTDLVGKTPPRCIRAPHTLAWPVFADLDNEEESPEICRKCGKYANGFGVFDLTDLGDVCLCSIRPQRHVGGPCCLCNKQYIRACGRYCARVLKHYKAFNKVIPCLHSRQVKPVFEGEVEDLFVELGAPTRMNFTEAELASQGASIMDRFVDLVEPCLSTEDSNFLDNICSDASIRKRLEDEYDVDMIAAARARKDFAKTLKLALQDRERKPDKWYSKLGCITTKGRQWAKKVVHGAKKLSDPLKTLAAILLVALHNCVAVDTTTMLSHFKPVNLLAILLDWTNDLPGFLTTLIRFMELYGVVQSTVNLVVDAIKSFWDRVMCATERCCDLLKRLFDKFEDSVPTGPTAGCLIFMSFVFSVIVGYLPNNSVISTFMKGAGKLTTFAGVIGAIRTLWITINQHMVAKDITSIQEKVMAVVKMANEAATLNQLEIVSVLCSELESTLTNRCTLPSYNQHMGVLNAAQKVVADIHTLVLGKINMTKQRPQPVAVVFKGAPGIGKTYLVHRLAKDLGCPHPSNINFGLDHFDSYTGEDVAIADEFNTSGDERWVELFIQMVNTNPCPLNCDKVENKNKVFSSKYLLCTTNSSMVLNATHPRATAFYRRVIIVDVRNKAVEGWQSTRHGSKPGKHCYTKDMSHLTFQVYPHNMPAPGFVFVGEKLVKSQVAPRELKYNELLDMIKNEHPDANFEGATKHEFVYPDVQYEQALLMWKQYFLMYGCTARLAKVFVDDIPYNQVHVARKSDPRSPGAVHHECELKYIWRMVPHFALGCVNMTNQLGTDLTQSQLDRITCGVEGITVTTVDNILPFHSQNTLINPSFLKLIWALRRHLRGLRGITQVATFIWKVMCNPVCAYDTLIRTLTGAATFSEDPVTTTIVCPNCTIQIHTCGGLLVRYSGDPAPVASDNVDRGNQGIDCLTNPNLIAGFSWRQIADLFSTVMTSLCNNHLVNLATMAAIGAVATKALQGVKGKTKRGRGARINLGNDEYDEWQQMRREFNNAHDMTAEEFLELRNRAAMGSDDADAIKFRSWWTNRQLRQDEAHVTVVGKGGVRNEVIRTRVRNAPKGPRTLDDGGFYDNDYEGLPGYLRFNGSGWMIHIGNGMYLSNTHTARSSCSEIVTCSPTTDLCLVKAEPIRSVAQIAEGTPVRDWKRASITTYGLKKTFSDSTKIDVLAYDGPTQTTHGDCGLPLFDEAGKVVAIHTGKLLGFSKMCTLIDCTITKGVYENTDLFCGDPIDYRGLVAFRVAGVEPRPPVSGTRYAKVPGVPEEYHTGYRPANLGRGDPDSHCTLMNIAVKNLQVYQQEPKLTKVDTFIERAAADVLGFLRFLTKGERQMNLNFSAAFNVLDLSTSCGPFVPGKKIDHVKDGKLDEVLSKHLYKCWSVANSGKALHHVYACGLKDELRPLDKVKEGKKRLLWGCNVGVALCAAAVFHNLCFKLKTVARFGPIAVGIDMTSRDVDVMITQLTSKAGDFLCLDYSKWDSTMSPCVVRLAIDILADCCEQTELTKSVVLTLKSLPMTVLDAMIVPTKRGLPSGMPFTSVINSICHWLLWSAAVYKACDEIGLFCSNLYEDAPFFVYGDDGVYAMTPMMVSLLPAILDNLRDYGLSPTAADKTEFIDVCPLKDISFLKRKFVMSELGWLSQLDRSSILRQLEWTKTAKRHMCIEECSELDKDERGVQLEELQIHAAAHGEEFFELVKKELRRQQAFTRFSVFDYQTARKTLGDRKRIVSVVPDDSFVNVMEGKPRADAPGTATTASVPGTTTDGMDPGVVASTDVVTADNVAASVATAGIGGPPQQASPQESWRVNFFYNDVFTWSVTDAPGSILYTVQHSPQNNPFTQVLSQMYAGWAGGMQFRFIVAGSGIFGGRLVCAIIPPGIQIQPGLEVRQFPHVVIDARSLEPVTITMPDLRPEMYHPTGNPGLVPTLVVSVYNNLINPFGGTTSAIQVTVETRPSEDFEFVLIRAPSSKTVDSVNPSWLLTTPVLTGAGSDNRWGAPIVGLQPVPGGFSTSNRHWNMNGETYGWSSPRFDDIDHPSGNVSYPSGSATNTIETWYANAGTATTNPISNIAPDGFPDMGAIPFSGTTIPTGAWVGFGQVWNASNGTPYVGTVQAYELGFANGAPSSIRPVTTTTGAQLVAKSIYGVAIAQNQTSAGIIFLSKGMVSTPGVAATTYTPQPSAIVTTPGTPVAAPIGKNTPIMFSAVVRRTGDVNAGPGSANGTQYGVGSQPLSVTLGLSLTNYSSALQPGQFFVWQLNFASGFMEVGMNTDGYFYAGTGAYSGMIDLTDLIDVRPVGVRPNTSTLVFNLAGVATTGYSYV

SEQ ID NO: 128 - protein, 2344 aa, synthetic construct
MAAMSRLTGMTTAILPEKKPLSFFLDLRDKTPPCCIRATGRLAWPVFPGQNGKEGPLETCNMCGKWLNGFGNFGLEDLGDVCLRSIAQQKHKFGPVCLCNRVYLHDCGRWRRRSRFLKHYKALNKVIPCAYQFVESFPTPIFEGEVDDLFVELGAPTSMGFMDKKLLKKGKKLMDKFVDVDEPCLTSRDASLLDSIASDNTIRAKLEEEYGVEMVQAARDRKDFMKNLRLALDNWPANPVTWYRKLGNITKKGKQWAKKVVYGARKVTDPLKTLASILLVGLHNVIAVDTTVMLSTFNPVNLLAILMDWNNDLTGFFATLVRLLELYGVVQATVNLIIEGVKSFWDKVVCATDRCFDLLKRLFDTFEDSVPTGPTAGCLIFMAFVFSTVVGYLPNNSVITTFMKGAGKLTTFAGVVGAIRTLWITINQHMVAKDLTSVQQKVMTVVKMANEAATLDQLEIVSCLCSDLETTLTNRCTLPSYNQHLGILNASQKVISDLHTMVLGKINTSKQRPQPVAVIFKGAPGIGKTYLVHRIARDLGCQHPSTINFGLDHFDSYTGEEVAIADESNTCGDGESWVELFIQMVNTNPCPLNCDKAENKNKVFNSKYLLCTTNSNMILNATHPRAGAFYRRVMIVEARNKAVESWQATRHGSKPGKSCYNKDMSHLTFQVYPHNMPAPGFVFVGDKLVKSQVTPREYKYSELLDLIKSEHPDVASFEGANKFNFVYPDAQYDQALLMWKQYFVMYGCVARLAKNFVDDIPYNQVHISRASDPKIEGCVEYQCKFQHLWRMVPQFVLGCVNMTNQLGTPLTQQQLDRITNGVEGVTVTTVNNILPFHSQTTLINPSFIKLIWAVRKHLKGLSGVTKVAQFIWRVMTNPVDAYGTLVRTLTGAATFSDDPVSTTIICSNCTIQLHSCGGLLVRYSRDPVPVASDNVDRGDQGVDVFTDPNLISGFSWRQIAHLFVEVISHLCANHLVNLATMAALGAVATKAFQGVKGKTKRGRGARVNLGNDEYDEWQAARREFVNAHDMTAEEYLAMKNKAAMGSDDQDSIMFRSWWTRRQLRPDEDQVTIVGRSGVRNEVIRTRVRQTPRGPKTLDDGGFYDNDYEGLPGFMRHNGSGRMIHIGNGLYISNTHTARSSCSEIVTCSPTTDLCLVKGEAIRSVAQIAEGTPVCDWKKSPISTYGIKKTLSDSTKIDVLAYDGCTQTTHGDCGLPLYDSSGKIVAIHTGKLLGFSKMCTLIDLTITKGVYETSNFFCGEPIDYRGITAHRLVGAEPRPPVSGTRYAKVPGVPDEYKTGYRPANLGRSDPDSDKSLMNIAVKNLQVYQQEPKLDKVDEFIERAAADVLGYLRFLTKGERQANLNFKAAFNTLDLSTSCGPFAPGKKIDHVKDGVMDQVLAKHLYKCWSVANSGKALHHIYACGLKDELRPLDKVKEGKKRLLWGCDVGVAVCAAAVFHNICYELKMVARFGPIAVGVDMTSRDVDVIINNLTSKASDFLCLDYSKWDSTMSPCVVRLAIDILADCCEQTELTKSVVLTLKSHPMTILDAMIVQTKRGLPSGMPFTSVINSICHWLLWSAAVYKSCAEIGLHCSNLYEDAPFYTYGDDGVYAMTPMMVSLLPAIIENLRDYGLSPTAADKTEFIDVCPLNKISFLKRTFELTDIGWVSKLDKSSILRQLEWSKTTSRHMVIEETYDLAKEERGVQLEELQVAAAAHGQEFFNFVCGELVRQQAYTQFSVYSYDAARKILADRKRVVSVVPDDEFVNVMEGKARAAPQGEAARTATTASVPGTTTDGMDPGVVATTSVITAENSSASIATAGIGGPPQQVDQQETWRTNFYYNDVFTWSVADAPGSILYTVQHSPQNNPFTAVLSQMYAGWAGGMQFRFIVAGSGVFGGRLVRAVIPPGIEIGPGLEVRQFPHVVIDARSLEPVTITMPDLRPNMYHPTGDPGLVPTLVLSVYNNLINPFGGSTSAIQVTVETRPSEDFEFVMIRAPSSKTVDSISPAGLLTTPDLTGVGNDNRWNGQIVGLQPVPGGFSTCNRHWNLNGSTYGWSSPRFADIDHRKGSASYPGSNATNVLQFWYANAGSAIDNPISQVAPDGLPDMSFVPFNGPGIPAAGWVGFGAIWNSNSGAPNVTTVQAYELGFATGAPGNLQPTTNTSGAQTVAKSIYAVVTGTAQNPAGLFVMASGIISTPNASAITYTPQPDRIVTTPGTPAAAPVGKNTPIMFASVVRRTGDVNATAGSANGTQYGTGSQPLPVTIGLSLNNYSSALMPGQFFVWQLTFASGFMEIGLSVDGYFYAGTGASTTLIDLTELIDVRPVGPRPSKSTLVFNLGGTANGFSYV

SEQ ID NO: 129 - protein, 2344 aa, synthetic construct
MAAMSRLTGMTTAILPEKKPLNFFLDLRDKTPPCCIRATGKLAWPVFLGQNGKEGPLETCNKCGKWLNGFGCFGLEDLGDVCLCSIAQQKHKFGPVCLCNRAYIHDCGRWRRRSRFLKHYKALNKVIPCAYQFDESFSTPVFEGEVDDLFVELGAPTSMGFMDKKLLKKGKKLMDKFVDVDEPCLTSRDASLLDSIASDNTIRAKLEEEYGVEMVQAARDRKDFMKNLRLALDNRPANPVTWYTKLGNITEKGKQWAKKVVYGACKVTDPLKTLASILLVGLHNVIAVDTTVMLSTFKPVNLLAILMDWTNDLTGFVTTLVRLLELYGVVQATVNLIVEGVKSFWDKVVCATDRCFDLLKRLFDTFEDSVPTGPTAGCLIFMAFVFSTVVGYLPNNSVITTFMKGAGKLTTFAGVIGAIRTLWITINQHMVAKDLTSIQQKVMTVVKMANEAATLDQLEIVSCLCSDLENTLTNRCTLPSYNQHLGILNASQKVISDLHTMVLGKINMTKQRPQPVAVIFKGAPGIGKTYLVHRIARDLGCQHPSTINFGLDHFDSYTGEEVAIADEFNTCGDGESWVELFIQMVNTNPCPLNCDKAENKNKVFNSKYLLCTTNSNMILNATHPRAGAFYRRVMIVEARNKAVESWQATRHGSKPGRSCYSKDMSHLTFQVYPHNMPAPGFVFVGDKLVKSQVAPREYKYSELLDLIKSEHPDVASFEGANRFNFVYPDAQYDQALLMWKQYFVMYGCVARLAKNFVDDIPYNQVHISRASDPKIEGCVEYQCKFQHLWRMVPQFVLGCVNMTNQLGTPLTQQQLDRITNGVEGVTVTTVNNILPFHSQTTLINPSFIKLIWAVRKHLKGLSGVTKVAQFIWRVMTNPVDAYGSLVRTLTGAATFSDDPVSTTIICSNCTIQIHSCGGLLVRYSRDPVPVASDNVDRGDQGVDVFTDPNLISGFSWRQIAHLFVEVISHLCANHLVNLATMAALGAVATKAFQGVKGKTKRGRGARVNLGNDEYDEWQAARREFVNAHDMTAEEYLAMKNKAAMGSDDQDSVMFRSWWTRRQLRPDEDQVTVVGRGGVRNEVIRTRVRQTPKGPKTLDDGGFYDNDYEGLPGFMRHNGSGWMIHIGNGLYISNTHTARSSCSEVVTCSPTTDLCLVKGEAIRSVAQIAEGTPVCDWKKSPISTYGIKKTLSDSTKIDVLAYDGCTQTTHGDCGLPLYDSSGKIVAIHTGKLLGFSKMCTLIDLTITKGVYETSNFFCGEPIDYRGITAHRLVGAEPRPPVSGTRYAKVPGVPEEYKTGYRPANLGRSDPDSDKSLMNIAVKNLQVYQQEPKLDKVDEFIERAAADVLGYLRFLTKGERQANLNFKAAFNTLDLSTSCGPFVPGKKIDHVKDGVMDQVHAKHLYKCWSVANSGKALHHIYACGLKDELRPLDKVKEGKKRLLWGCDVGVAVCAAAVFHNICYKLKMVARFGPIAVGVDMTSRDVDVIINNLTSKASDFLCLDYSKWDSTMSPCVVRLAIDILADCCEQTELTKSVVLTLKSHPMTILDAMIVQTKRGLPSGMPFTSVINSICHWLLWSAAVYKSCAEIGLHCSNLYEDAPFYTYGDDGVYAMTPMMVSLLPAIIENLRDYGLSPTAADKTEFIDVCPLNKISFLKRTFELTDIGWVSKLDKSSILRQLEWSKTTSRHMMIEETYDLAKEERGVQLEELQVAAAAHGQEFFNFVCKELERQQAYTQFSVYSYDAARKILADRKRVVSVVPDDEFVNVMEGKARTAPQGEAAGTATTASVPGTTTDGMDPGVVATTSVVTAENSSASIATAGIGGPPQQVDQQETWRTNFYYNDVFTWSVADAPGSILYTVQHSPQNNPFTAVLSQMYAGWAGGMQFRFIVAGSGVFGGRLVAAVIPPGIEIGPGLEVRQFPHVVIDARSLEPVTITMPDLRPNMYHPTGDPGLVPTLVLSVYNNLINPFGGSTSAIQVTVETRPSEDFEFVMIRAPSSKTVDSISPAGLLTTPVLTGVGNDNRWNGQIVGLQPVPGGFSTCNRHWNLNGSTYGWSSPRFADIDHRRGSASYPGNNATNVLQFWYANAGSAIDNPISQVAPDGFPDMSFVPFNGPGIPAAGWVGFGAIWNSNSGAPNVTTVQAYELGFATGAPGNLQPTTNTSGSQTVAKSIYAVVTGTAQNPAGLFVMASGVISTPSANAITYTPQPDRIVTTPGTPAAAPVGKNTPIMFASVVRRTGDVNATAGSANGTQYGTGSQPLPVTIGLSLNNYSSALMPGQFFVWQLTFASGFMEIGLSVDGYFYAGTGASTTLIDLTELIDVRPVGPRPSKSTLVFNLGGTANGFSYV

SEQ ID NO: 130 - protein, 1699 aa, synthetic construct
MKMASNDASAAAVVNSNNDTAKSSSDGVLSSMAVTFKRALGGRAKQPPPRETPQRPPRPPTPELVKKIPPPPPNGEDELVVSYSVKDGVSGLPELSTVRQPDEANTAFSVPPLNQRENRDAKEPLTGTILEMWDGEIYHYGLYVERGLVLGVHKPPAAISLAKVELTPLSLFWRPVYTPQYLISPDTLKRLHGESFPYTAFDNNCYAFCCWVLDLNDSWLSRRMIQRTTGFFRPYQDWNRKPLPTMDDSKLKKVANVFLCALSSLFTRPIKDIIGKLRPLNILNILASCDWTFAGIVESLILLAELFGVFWTPPDVSAMIAPLLGDYELQGPEDLAVELVPIVMGGIGLVLGFTKEKIGKMLSSAASTLRACKDLGAYGLEILKLVMKWFFPKKEEANELAMVRSIEDAVLDLEAIENNHMTTLLKDKDSLATYMRTLDLEEEKARKLSTKSASPDIVGTINALLARIAAARSLVHRAKEELSSRLRPVVVMISGKPGIGKTHLARELAKRIAASLTGDQRVGLIPRNGVDHWDAYKGERVVLWDDYGMSNPIHDALRLQELADTCPLTLNCDRIENKGKVFDSDAIIITTNLANPAPLDYVNFEACSRRIDFLVYAEAPEVEKAKRDFPGQPDMWKNAFSPDFSHIKLALAPQGGFDKNGNTPHGKGVMKTLTTGSLIARASGLLHERLDEYELQGPALTTFNFDRNKVLAFRQLAAENKYGLMDTMKVGRQLKDVRTMPELKQALKNISIKRCQIVYSGCTYTLESDGKGNVKVDRVQSATVQTNHELAGALHHLRCARIRYYVKCVQEALYSIIQIAGAAFVTTRIVKRMNIQDLWSKPQVEDTEDTANKDGCPKPKDDEEFVVSSDDIKTEGKKGKNKTGRGKKHTAFSSKGLSDEEYDEYKRIREERNGKYSIEEYLQDRDKYYEEVAIARATEEDFCEEEEAKIRQRIFRPTRKQRKEERASLGLVTGSEIRKRNPDDFKPKGKLWADDDRSVDYNEKLDFEAPPSIWSRIVNFGSGWGFWVSPSLFITSTHVIPQGAQEFFGVPVKQIQIHKSGEFCRLRFPKPIRTDVTGMILEEGAPEGTVVTLLIKRSTGELMPLAARMGTHATMKIQGRTVGGQMGMLLTGSNAKSMDLGTTPGDCGCPYIYKRENDYVVIGVHTAAARGGNTVICATQGSEGEATLEGGDNKGTYCGAPILGPGSAPKLSTKTKFWRSSTAPLPPGTYEPAYLGGKDPRVKGGPSLQQVMRDQLKPFTEPRGKPPKPSVLEAAKRTIINVLEQTIDPPQKWSFTQACASLDKTTSSGHPHHMRKNDCWNGESFTGKLADQASKANLMFEEGKNMTPVYTGALKDELVKTDKIYGKIKKRLLWGSDLATMIRCARAFGGLMDELKAHCVTLPIRVGMNMNEDGPIIFERHSRYKYHYDADYSRWDSTQQRAVLAAALEIMVKFSPEPHLAQIVAEDLLSPSVMDVGDFKISINEGLPSGVPCTSQWNSIAHWLLTLCALSEVTNLSPDIIQANSLFSFYGDDEIVSTDINLNPEKLTAKLKEYGLKPTRPDKTEGPLIISEDLNGLTFLRRTVTRDPAGWFGKLDQSSILRQMYWTRGPNHEDPSETMIPHSQRPIQLMSLLGEAALHGPAFYSKISKLVIAELKEGGMDFYVPRQEPMFRWMRFSDLSTWEGDRNLAPSFVNEDGVE

SEQ ID NO: 131 - protein, 1789 aa, synthetic construct
MMMASKDVVPTAASSENANNNSSIKSRLLARLKGSGGATSPPNSIKITNQDMALGLIGQVPAPKATSVDVPKQQRDRPPRTVAEVQQNLRWTERPQDQNVKTWDELDHTTKQQILDEHAEWFDAGGLGPSTLPTSHERYTHENDEGHQVKWSAREGVDLGISGLTTVSGPEWNMCPLPPVDQRSTTPATEPTIGDMIEFYEGHIYHYAIYIGQGKTVGVHSPQAAFSITRITIQPISAWWRVCYVPQPKQRLTYDQLKELENEPWPYAAVTNNCFEFCCQVMCLEDTWLQRKLISSGRFYHPTQDWSRDTPEFQQDSKLEMVRDAVLAAINGLVSRPFKDLLGKLKPLNVLNLLSNCDWTFMGVVEMVVLLLELFGIFWNPPDVSNFIASLLPDFHLQGPEDLARDLVPIVLGGIGLAIGFTRDKVSKMMKNAVDGLRAATQLGQYGLEIFSLLKKYFFGGDQTEKTLKDIESAVIDMEVLSSTSVTQLVRDKQSARAYMAILDNEEEKARKLSVRNADPHVVSSTNALISRISMARAALAKAQAEMTSRMRPVVIMMCGPPGIGKTKAAEHLAKRLANEIRPGGKVGLVPREAVDHWDGYHGEEVMLWDDYGMTKIQEDCNKLQAIADSAPLTLNCDRIENKGMQFVSDAIVITTNAPGPAPVDFVNLGPVCRRVDFLVYCTAPEVEHTRKVSPGDTTALKDCFKPDFSHLKMELAPQGGFDNQGNTPFGKGVMKPTTINRLLIQAVALTMERQDEFQLQGPTYDFDTDRVAAFTRMARANGLGLISMASLGKKLRSVTTIEGLKNALSGYKISKCSIQWQSRVYIIESDGASVQIKEDKQALTPLQQTINTASLAITRLKAARAVAYASCFQSAITTILQMAGSALVINRAVKRMFGTRTAAMALEGPGKEHNCRVHKAKEAGKGPIGHDDMVERFGLCETEEEESEDQIQMVPSDAVPEGKNKGKTKKGRGRKNNYNAFSRRGLSDEEYEEYKKIREEKNGNYSIQEYLEDRQRYEEELAEVQAGGDGGIGETEMEIRHRVFYKSKSKKHQQEQRRQLGLVTGSDIRKRKPIDWTPPKNEWADDDREVDYNEKINFEAPPTLWSRVTKFGSGWGFWVSPTVFITTTHVVPTGVKEFFGEPLSSIAIHQAGEFTQFRFSKKMRPDLTGMVLEEGCPEGTVCSVLIKRDSGELLPLAVRMGAIASMRIQGRLVHGQSGMLLTGANAKGMDLGTIPGDCGAPYVHKRGNDWVVCGVHAAATKSGNTVVCAVQAGEGETALEGGDKGHYAGHEIVRYGSGPALSTKTKFWRSSPEPLPPGVYEPAYLGGKDPRVQNGPSLQQVLRDQLKPFADPRGRMPEPGLLEAAVETVTSMLEQTMDTPSPWSYADACQSLDKTTSSGYPHHKRKNDDWNGTTFVGELGEQAAHANNMYENAKHMKPIYTAALKDELVKPEKIYQKVKKRLLWGADLGTVVRAARAFGPFCDAIKSHVIKLPIKVGMNTIEDGPLIYAEHAKYKNHFDADYTAWDSTQNRQIMTESFSIMSRLTASPELAEVVAQDLLAPSEMDVGDYVIRVKEGLPSGFPCTSQVNSINHWIITLCALSEATGLSPDVVQSMSYFSFYGDDEIVSTDIDFDPARLTQILKEYGLKPTRPDKTEGPIQVRKNVDGLVFLRRTISRDAAGFQGRLDRASIERQIFWTRGPNHSDPSETLVPHTQRKIQLISLLGEASLHGEKFYRKISSKVIHEIKTGGLEMYVPGWQAMFRWMRFHDLGLWTGDRDLLPEFVNDDGV

SEQ ID NO: 132 - protein, 1788 aa, synthetic construct
MMMASKDVVATNVASNNNANNTSATSRFLSRFKGLGGGASPPSPIKIKSTEMALGLIGRTTPEPTGTAGPPPKQQRDRPPRTQEEVQYGMGWSDRPIDQNVKSWEELDTTVKEEILDNHKEWFDAGGLGPCTMPPTYERVKDDSPPGEQVKWSARDGVNIGVERLTTVSGPEWNLCPLPPIDLRNMEPASEPTIGDMIEFYEGHIYHYSIYIGQGKTVGVHSPQAAFSVARVTIQPIAAWWRVCYIPQPKHRLSYDQLKELENEPWPYAAITNNCFEFCCQVMNLEDTWLQRRLVTSGRFHHPTQSWSQQTPEFQQDSKLELVRDAILAAVNGLVSQPFKNFLGKLKPLNVLNILSNCDWTFMGVVEMVILLLELFGVFWNPPDVSNFIASLLPDFHLQGPEDLARDLVPVILGGIGLAIGFTRDKVTKVMKSAVDGLRAATQLGQYGLEIFSLLKKYFFGGDQTERTLKGIEAAVIDMEVLSSTSVTQLVRDKQAAKAYMNILDNEEEKARKLSAKNADPHVISSTNALISRISMARSALAKAQAEMTSRMRPVVIMMCGPPGIGKTKAAEHLAKRLANEIRPGGKVGLVPREAVDHWDGYHGEEVMLWDDYGMTKILDDCNKLQAIADSAPLTLNCDRIENKGMQFVSDAIVITTNAPGPAPVDFVNLGPVCRRVDFLVYCSAPEVEQIRRVSPGDTSALKDCFKLDFSHLKMELAPQGGFDNQGNTPFGKGTMKPTTINRLLIQAVALTMERQDEFQLQGKMYDFDDDRVSAFTTMARDNGLGILSMAGLGKKLRGVTTMEGLKNALKGYKISACTIKWQAKVYSLESDGNSVNIKEERNILTQQQQSVCTASVALTRLRAARAVAYASCIQSAITSILQIAGSALVVNRAVKRMFGTRTATLSLEGPPREHKCRVHMAKAAGKGPIGHDDVVEKYGLCETEEDEEVAHTEIPSATMEGKNKGKNKKGRGRRNNYNAFSRRGLNDEEYEEYKKIREEKGGNYSIQEYLEDRQRYEEELAEVQAGGDGGIGETEMEIRHRVFYKSKSRKHHQEERRQLGLVTGSDIRKRKPIDWTPPKSAWADDEREVDYNEKISFEAPPTLWSRVTKFGSGWGFWVSPTVFITTTHVIPTSAKEFFGEPLTSIAIHRAGEFTLFRFSKKIRPDLTGMILEEGCPEGTVCSVLIKRDSGELLPLAVRMGAIASMRIQGRLVHGQSGMLLTGANAKGMDLGTIPGDCGAPYVYKRANDWVVCGVHAAATKSGNTVVCAVQASEGETTLEGGDKGHYAGHEIIKHGCGPALSTKTKFWKSSPEPLPPGVYEPAYLGGRDPRVTVGPSLQQVLRDQLKPFAEPRGRMPEPGLLEAAVETVTSSLEQVMDTPVPWSYSDACQSLDKTTSSGFPYHRRKNDDWNGTTFVRELGEQAAHANNMYEQAKSMKPMYTGALKDELVKPEKVYQKVKKRLLWGADLGTVVRAARAFGPFCDAIKSHTIKLPIKVGMNSIEDGPLIYAEHSKYKYHFDADYTAWDSTQNRQIMTESFSIMCRLTASPELASVVAQDLLAPSEMDVGDYVIRVKEGLPSGFPCTSQVNSINHWLITLCALSEVTGLSPDVIQSMSYFSFYGDDEIVSTDIEFDPAKLTQVLREYGLRPTRPDKSEGPIIVRKSVDGLVFLRRTISRDAAGFQGRLDRASIERQIYWTRGPNHSDPFETLVPHQQRKVQLISLLGEASLHGEKFYRKISSKVIQEIKTGGLEMYVPGWQAMFRWMRFHDLGLWTGDRNLLPEFVNDDGV

SEQ ID NO: 133 - protein, 3027 aa, synthetic construct
MSSSNSQNSVNMVDGVDLNDPTAVAIAAASGTWGELDRASTCMYNFFDVSDVERNPGESSKQLVSRIKKRAGALLGVSRAFKDTEQVLSAERCAFKSFSDDEEIMATTFEPLKDRAEITPTAASKLDKTLEAHRAKFNYMAIDSIRVAVTSLMHQGDSRECIMYLCDRRFKDPLLGAIALIGFTLPGMQTHVYKTGRMMAFSRKEAIAADRLQLYLYVKGAKLERTQNTPITVNVRTSLIFGSNAENLLKCDSQIDTDMNIVSFMAQNQDLFGWLEAAQGGGYVPQSLRSATTHTSNSVLLHKWNNPVGSVSQRIASRMIFTKAIDAGTSYEDSDVVGSNAKCPNSIGKNLRIGVAQASIQNTKDDSTPYSLADFVQDGPIAPVVASLESFMSSPSISQTLPLINDQFTRPIYSRTFEWKATDTVGASIFQLELPGDVVGPQASSLFSDTMQRAFCFSSDFELSILLTGNESYMGALKIVTDQLRRFHEAKQDDARVFHSMPGRTVFAKDSDGIKIPIEFMSIHKAVSAHDSNSHNALSRVEARVVTPLSHISLSSPVLSITIQVFAKNVKADYMMWRSLETTFPTANATLPSAVGDNFGRLRTSQSEILSTSQILGLLTERAFLGTAKVQQDTGARVIIAEFALHPMSSRNVDGTLLLSQLAALSAMYAFWRGSLVLTFEINCSASTRGKLIVSVTPKGGVALAGITASHQGYGAEFDLGTSSTRSFTMPFVSTDEWESIGDDGIMSAFEGVWDCPVANLLVLHPITSIAESTPSVDIRCYLHPGPDFQLRGRRHIGLRAASRPLSIAQASPVLSQVDFSLMASISIDATEESVVVAVPCAPWYSKEEVDYTLLQNPLHWASRMFTLWRGDIEYRFVVKEEALGDGWQSPISVWHNPNTQLSKCKITKISNKKISKETYHGKKFCLMQLKSIDIVAVDDRRFSWRLCKILDTTKDTAGDSTSPSVTQITYTGHPPMSSQTGVVCIKFPKNSIKGKLKVYSKPGENFEFRHLGGVPSLQVSQMVKYKKPFQNSVPDVFITPSKESSKKELGFKPKVVESAAVPKLAVGQAQGLVSKIKGFGSLWKFDEEEETLNSELQKMAVEVSGEIDPIQDEGWAKRKINEIVSSVSTKLIEASTSILSNAATRAISTLFDVMIGKVRGVLSSLVDSISGAFKMCLGDPKCLCLIGISISAVLGYCTLKLVENSVPDALGIFKALMMVAITSISALYWPKAAISIVTKYEEQFKDIENYCSTIYKHIFLGVSEDKMEGATPAKACATNFEDLAHGKAQAGGKSFLELAGLIAYIRLCVVLCKAMNTSFLEPFTPSNMEKQCRTVGGISIGVKTLCEFKDYIYRMIVGGITPTSSYVKVSGLTGFDIREWFEEVESVTLQETRYTQMGSDEKIKQIRALYDKGVNVMGKLTMIDSPHLSRVCERSFRLCKELLDETHRCKGASSTRVDPFHVSLYGSPGVGKSFVMGKLLDDVLDFMSEPQADRCYSKTPNEEYWSGYIGQTAVKCDDLGQDLSKGFSPTYNQIIQMKTNNCFIVPMADLANKGRTFTSKYIFSTTNVPGCGTKHGLADPGAFMRRRNIFVEVETEGDMIPGSTNHMRFTLLNPLNPDERIMKYPARMKYVDFLCVCVAEARVYFETQNLVMETLNGTTKNQEEPSKDVIAILEELGDGVVEGILEKRKELLSQFGVMDPPPFDAIELEPGKAQASVCFSTDAFGNPLKNPFVELFGKLRDEFERATKQEMPDDILTKFGASLTLGEPTVFGYENQCGMHSAKDSNLMSSFFTFIFGKNLIYKQEQEFLRHIDTLSSMGVLRLVDAVTTTTKGDKKILSFANIYDNQAFEQLGVLERLIFHLVLATRAAKKGRINGIRERLANWFTSARILSNNILEELPSPIKMLLVLATSVGSLYLAFKGLSGIGSMILGFTGNFTAKEEDFEMISLNALMGQAKSKGRNFITSGDELTTRLSRMMSRASLATGRAQGGRSHMDTCEALLARQGQITNMATGLHLVATDLGGGFLLAPLHTFAGAEKDDIFRFQNGADYYFAFEPKDVSQLSEYDACIIRTDAIPMKSSIVSIFAKESQIELLVDMSAHFVCGPWKVPFGGEFISEQTVAKRIASFKYFMDEKLYMAINGWSSPFKTEDGQCGSCLVSTSDKLDGKVFCSLVAGTYDRVTGKYVSTYVPITCDMIKKSISLLTGAEFSESQSSICDSPISDTVAETIKVDQLFSSKPGASGKFGVFGVNDTIGIIDVVGRTFPETTPKSITKSTIVPSLIQPYMPRKPLTEPAILDPRDVRLGENRYDPMIDGIKKYEEQARPIKISWRNQIIESMAAQMQDWETFMVREGYMTMDLPMSVVINGIDGVEYYEPLNMSTSEGYPLILNRPKDAHGKEYLFETMESGERRIKSAKLEAHYESYGHALQSTEPFPLICIECPKDERRALDKIYEKPKTRLFSILPVEFNMHARRLFLDFNVFVMANRHKHGIMVGINPHSREWSDLAISLASFSPYGFNGDFANFDGMFHPSSFSMVSELANIFYGNFLSTERDNLTRMLTNRFSLMKGAILRVPGGGPSGFPMTVIFNSFINLFYLQSAWIMLARFNGRQDISHPCNFPKYVRACVYGDDNIVAIKMEVLPWYNLQTVSEALFDYFGVTMTDGAKNKASEAKPYGKILEFDFLKRHFKADELIPSLFHAPLHKRSIEEQVYWIREGGNSLELLEANIENALYEAHHHGREYYEELKDQIKKAMNRAGYMSFVAPSYLMCRQRWLQQDLGEVATSSLPSHVGLLKEATKNHFSALTGQEEIKAIFEEIDNGNGGTTKHGNMQQILPNIFIGPTRIFETKYGNSLFNLVCDNSLSKGQTRYGVKHGIQSLSKPDFTYISESLPCLTTPNFRMVCLDPIGGELALATALCLLHAAGIINTKTFTMFMRIHIKQWKHVLQAYFRVCETFVSKEWQNFKRDIKRLSQDDVGCSRTTPVCGRFLTLDGQLPQHIKSLDKIDFKKTRRIKIAQDEDFIIQID

SEQ ID NO: 134 - protein, 3473 aa, synthetic construct
MQSFLLSSKNQAKLLHAGLEFVGGVRCAHQGWVSGKAVVFCNYCNFAHRLYRFYTKNHCVLNKELLKISVEGLLCHCIEQAFLFRRFYDRRFAWQRKYAKGFLFDNLSIPFDDCALCPNAGTRLSQTGVSHDHFVCNYVEHLFECASFSRETGGKFFRACSEGWHWNATCTTCGASCRFANPRENIVIAIFMNFLRVMYDGNKYYVSLHCDTEWIPVHPLFARLVLMVRGFAPLDNSHVIEEDEMDICGHSSEVTYEDPSKFAFTHQHVTRGVGMGHLAFCRDANGVDRGEHKFYLHGPFDLKMTHAMFRVFMILLNCHGYVQSEFRDEFPDIKDRSLCGLLSVAGLRGVNVSCNEEFIHLHSQFHNGSFRSQRPIPMVYAEPEMYPPLGYVHLTESWVPRGRLLIDDLPSLMSRVYAESSQAQAGEIYEETFDEDDLFELDGEEGTSTRGLLDLGRRLGGLLLGATKCVKGLHSVIEWPVDVLTKEAEDLGTWLADNKKYVSESTWSCQVCPEVQDALEKSMREQAKLNAQMISGIKKLATTMDSATLKLRDNLKELEQRISVLEQGADDTQQVRITNLENFCEDAAKAFEALRNDIEALKKKPAQSVTPLPSPSGNSGTAGEQRPPPRRRRRPPVVEMSEAQAGETVIVGGDEEQEAHQDSSVAAAGPADEHNAMLQKIYLGSFKWKVSDGGGSILKTFSLPSDIWAANDRMKNFLSYFQYYTCEGMTFTLTITSIGLHGGTLLVAWDALSSATRRGIVSMIQLSNLPSMTLHASGSSIGTLTVTSPAIQHQICTSGSEGSLANLGSLVISVANVLCADSASAQELNVNAWVQFNKPKLSYWTAQHSIAQSGGFEESQDLGDLQAIIATGKWSTTSDKNLMEIIVHPTACYVSEKLIYQTNLSVVAHMFAKWSGSMRYTFVFGASMFDRGKIMVSAVPVQFRNSKLTLSQMAAFPSMVCDLSMETREFTFEVPYISIGKMSLVCKDYLFDISSYNADLVVSRLHVMILDPLVKTGNASNSIGFYVVAGPGKGFKLHQMCGVKSQFAHDVLTAQDFGRSLSCSRLLGNGFKEWCSRESLLMRVPLKSGKKRAFKYAVTPRMRTLPPEATSLSWLSQIFVEWRGSLTYTIHVQSGSAIQHSYMRIWYDPNGKTDEKEVKFLDSAHPPAGIKVYHWDLKIGDSFRFTVPYCARTEKLQIPKAYASTPYEWLTMYNGAVTFDLRSGADMELFVSIAGGDDFEMFEQTVPPKCGSVSDSYTVLSYADDVKSVTEVPNKTTYLADEQPTTSAPRTSIVNTEDDPPTEGEIARTTNGTLVQYRGGAWKPMVERTPTMSKKQVGPELTVSDPQMYKCIKNMNKNVKILTDRQCTAKLANIVDSAQELVGSNSTFVEDLAVGAKQIRKFGESLDVFEGSMSAAKTAELIDNTHAAFSGPADGSPISNVVQLLLPMLSSIKGMSGKMESKMASLTAMFQPCKKAITHLIERSFPYLACKGFKTDKWIWAALASILVGAALLHYYRSDLKFVKKWSVMCMIIWAPLLAEKAYHLGTWIKEKFLKSLPRTRTIKDSCRKHSLAGAFECLASASCAYIKDNWAKTMSSLLTILSVVASLVMWGKIPDDKEITSFADKFHSIGKKGRSITNIIGGFEKITSVCKKWSETLVSWIVSNVSGGIPKEDLAMTAYLGFKIHDWVRETRDMALMENRFRGFGGDEHLVKVRRLYGHSLKIDNALMEKQIVPDMQLSLIIKECRQKCLELMNESYTYKGMKQSRIDPLHVCMLGAPGVGKSTIAHVVINHLLDHRGEPEVDRIYTRSCADAYWSNYHQEPVILYDDLGAIKSNLRLSDYAEIMGIKTNDPFSVPMAAVEDKGKHCTSKYVFSCTNVLNLDDTGDVVTKMAYYRRRNVLVKVERDPDVPKNEANPTEGLVFTVLGHDQNCQGDPQFVVKENWDEPFLREVDTEGWRFERVEYRTFLRFLCMYTDAYMYSQEQVLQGIKTFKMNPFAPEPEFAQAQNGEAAECEIVEEMQEVPGEAPQEAKELVKIETAPNMDELVEAFNKLRVTPGHLNDILRDGSGCYIDEWAIAGPRWLSFHELLPFTCGCHHTRVCDFNIVYNNMCKAVRSQSVHFKYRANQAIKYAYTHKLHSQCRYSIDFEKLRECNPLDVFVCVLSKYTADDHSFERRCPKKMNVVRMQRPPVFELKMRPPSDSVVVEDEQGQRIFEWPHLYIFLRYRAIEFKDDKGSLTVREDAGADVCPWNEFLKLPWLDGDQLKSVLPAHLHRMVQARLEQVEIMEENGNYSGEMRNAIAEIKEYLDQDHQWVAALVLVACAVKERRRMTHDKLHRKSFNALDKLDAWYTTTAPKTSKKMKILLAIGASVAVAGVAVGAVILLQKTNLFGSKEDEEIEGEEGETQASGAHESDGIVTQHLKRDIRPKMRVTYTDHHVAEAHEEKDAEKPRKSGNPTRKSYLGLSPGFAERGMGVTYEEHTPLKDALLDESNKVFRRKIVASVESAVKQGGKASKDTVLSQIGDWQDKVKATGVIAARQLEASGSLKKIHNLNSRRTSSHVMPGLVVHDGAFERSDEVDAELHRITIDEVKSCPKMIKEGVSTLSVKKASVGMLALQKAESQLSFPFTSRAGVDRDLSMTNLIDTHMAGMSCIIISELGNVFRTFGVLRLCGTYVCMPAHYLDEITSEHTLYFVCPSKITQIQLERHRVCLVNGFQETVVWDLGPSVPPSRNYIDFIANADDWKNYKATSGALVMSKYSVDSMLQCVHFLDSIELTEANVSVPTSYYEANGGIHTIISGLRYRVHCMPGFCGRAIMRADATCFRKIIGMHVSGLRNKCMGYAETLTQEHLMQAIETLKETGLLKHIPKGAIGAGEEKLPEHSKKQSLSLEGKGNLGIVGQLTAQLVPTSVTKTTICKSMIHGLIGEIKTEPSVLSAWDRRLPFPPGEWDPMKDAVKKYGSYILPFPTEEIQEVENFLIKKFRRKENSRRTRNVNSLEVGINGIDGSDFWSPIEMKTSPGYPYILKRPSGAQGKKYLFEELEPYPSGRPKYAMKDPELIENYERIKEEVTSGVKPSIMTMECLKDERRKLAKIYEKPATRTFTILSPEVNILFRQYFGDFAAMVMSTRREHFSQVGINPESMEWSDLINSLLRVNTKGFAGDYSKFDGIGSPAIYHSIVNVVNAWYNDGEVNARARHSLISSIVHRDGICGDLILRYSQGMPSGFAMTVIFNSFVNYYFMALAWMSTVGSSLLSPQGSCKDFDTYCKIVAYGDDNVVSVHEEFLDVYNLQTVAAYLSHFGVTYTDGDKNPIHMSKPYEDITKMSFLKRGFERVESSGFLWKAPLDKTSIEERLNWIRDCPTPVEALEQNIESALHEAAIHGRDYFDDLVRRLNSALTRVMLPPTDISFEECQARWWASVTGALRAADYTSLVRRASSGHVEFNKKYRDMFRQQDLPLKEILMKSKPVALLDLEV

SEQ ID NO: 135 - protein, 3471 aa, synthetic construct
MQSFLLSSKNQAKLLHAGLEFVGGVRCAHQGWVSGKAVVFCNYCNFAHRLYRFYTKNHCVLNKETIENLCGRSFVSLYRAGLLLDDFTIDDLLGKGKYAKGSIDNLSIPFDDCALCPNAGTRLSQTGVSHDHFVCNYVEHLFECASFSRETGGKFIRACSKGWHWNATCTTCGASCRFANPRENVVIAIFMNFLRVMYDGNKYYVSLHCDTEWIPVHPLFARLVLMVRGFAPLDNSHVIEEDEMDICGHPSEVTYDDPSNYAFSHQHVTRGVGMGHLAFCRDANGVDRGEHKFYLHGPFDLKMTHAMFRVFMILLNCHGYVQSEFREEHPAVKDRSLCALLSVAGLRGVNIACNEEFIHLHSQFHNGSFRSQRPIPMVYAEPEMYPPLEYVRLTESWVPRGRVMIDDLPSLLSRVYAESSQPHAGEIYEEIFDEDDLFELGDDEGTSTRGLLDLGRRLGGLLLGATKCVKGLHAVIEWPVDVLTKEAEDLGTWLADNKKYVSESTWSCQVCPEVQDALEKSMREQAKLNAQVIGGIKKLATTMDSATSKLKDSLKELERRISVLEQGVDETQQARITNLENFCEDAAKAFDALRADIDALKKKPAQSVTPLPSPSGNSGTAGEQRPPPRRRPPVVEMSEAQAGETVIVGGDEEQEAHQDSSVAAAGPTDEHNAMLQKIYLGSFKWKVSDGGGSILKTFSLPSDIWAANDRMKNFLSYFQYYTCEGMTFTLTITSIGLHGGTLLVAWDALSSATRRGIVSMIQLSNLPSMTLHASGSSIGTLTVTSPAIQHQICTSGSEGSIANLGSLVISVANVLCADSASAQELNVNAWVQFDKPKLSYWTAQHTIAQSGGFEESQDLGDLQAIIATGKWSTTSDKNLMEIIVHPTACYVSEKLIYQTNLSVVAHMFAKWSGSMKYTFVFGASMFDRGKIMVSAVPVQFRNSKLTLSQMAAFPSMVCDLSVETREFTFEVPYISIGKMSLVCKDYLFDISSYNADLVVSRLHVMILDPLVKTGNASNSIGFYVVAGPGKDFKLHQMCGVKSQFAHDVLTAQDFGRSLSCSRLLGNGFKEWCSRESLLMRIPLKNGKKRAFKYAVTPRMRTLPPEATSLSWLSQIFVEWRGSLTYTIHVQSGSAIQHSYMRIWYDPNGKTDEKEIKFLDSAHPPAGIKVYHWDLKIGDSFRFTVPYCARTEKLQIPKAYASTPYEWLTMYNGAVTIDLRSGADMELFVSIAGGDDFEMFEQTVPPKCGSVSDSYTVLSYADDIKSVTEVPNKTTYLADEQPTTSAPRTSTVDTEEDPPTEGEIARTSNGTLVQYRGGAWKPMVERTPTMSKKQVGPELVASDSHMYKCIKNMNKNVKILTDRQCTAKLADIVDSTQGLVGSNSTFVEDLAVGAKQIRKFGESLEVFEGSMSAAKTAELIDNTHAAFSGPADGSPISNVVQLLLPMLSSIKGMSGKMESKMASLTAMFQPCKKAITHLIERSFPYLACKGFKTDKWIWAALASILVGAALLHYYRSDLKFVKKWSVMCMIIWAPLLAEKAYHLGTWIKEKFLKSLPRTRTIKDSCRKHSLAGAFECLASASCAYIKDNWAKTMSSLLTILSVVASLVMWGKIPDDKEITSFADKFHSIGKKGRSITNIIGGFEKITSVCKKWSETLVGWIVSNVSGGIPKEDLAMTAYLGFKIHDWVRETRDMALMENRFQGFGGDEHLVRVRRLYGHSLKIDNALMEKQIVPDMQLSLIIKECRQKCLELMNESYTYKGMKQSRIDPLHVCMLGAPGVGKSTIAHVVINNLLDHRGEPEVDRIYTRCCADAYWSNYHQEPVILYDDLGAIKSNLRLSDYAEIMGIKTNDPFSVPMAAVEDKGKHCTSKYVFSCTNVLNLDDTGDVVTKMAYYRRRNVLVKVERDPDVPKNEANPTEGLVFTVLGHDQNCQGDPQFVVKENWDEPFLREVDTEGWRFERVEYRTFLRFLCMYTDAYMYSQEQVLQGIKTFKMNPFAPEPEFAQAQSGEAAECEIVEETQEIPGEAPQEVKELAKIETAPNMDELVEAFNKLRVTPGHLNEILRDGSGCYIDEWAIAGPRWLSFHELLPFTCGCHHTRVCDFNIVYNNMCKAVRSQSVHFKYRANQAIKYAYTHKLHSQCRYSIDFEKLRECNPLDVFVCVLSKYTADDHSFERRCPKKMNVVRMQRPPVFELKMRPPSDSVVVEDDQGQRAFEWPHLYTFLRYRAIEFKDDKGSLTVREDASADVCPWNEFLKLPWLDGDQLKSVLPAHLHRMVQARLEQVEIMEENGNYSGEMRNAIAEIKEYLDQDHQWVAALVLVACAVKERRKMTHDKLHRKSFNALDRLDKWYTTTAPKTSKKMKILLAIGASVAVAGVAVGAVILLQKTNLFGSKEDEEIEGEEGETQASGAHESDGIVTQHLKRDIRPKMRVTYTDHHVAEAHEEKSTEKPRKPGNPTRKNFLGLSPGFAERGMGVTYEEHTPLKDALLDESNKVFRRKIVASVESAVKQGGKASKDSVLSQISEWQDKVRATGVIAARQLEASGSLKKIHNLNSRRTSSHVMPGLVVHDGTFERSDEVDAELHRITIDEVKSCPKMIKEGVSTLSVKKASVGVLALQKAESQLSFPFTSRAGVDRDLSMTNLIDTHMAGMSCIIISELGNVFRTFGVLRLCGTYVCMPAHYLDEITSEHTLYFVCPSKITQIQLERHRVCLVNGFQETVVWDLGPSVPPSRNYIDFTAKADDWKNYKATSGALVMSKYLVDSMLQCVHFLDSIELTEANVSVPTSYYEANGGIHTIISGLRYRVHCMPGFCGAAIMRADATCYRKIIGMHVSGLRNKCMGYAETLTQEHLMRAIETLKETGLLKHIPRGAIGAGEEKLPEHSKKQSLSLEGKGNLGIVGQLPAQLVPTSVTKTTICKSMIHGLIGEIKTEPSVLSAWDRRLPFPPGEWDPMKDAVKKYGSYILPFPTEEIQEVENFLIKKFRRKENSRRTRNVNSLEVGINGIDGSDFWSPIEMKTSPGYPYILKRPSGAQGKKYLFEELEPYPSGRPKYAMKDPELIENYERIKEEVTSGVKPSIMTMECLKDERRKLAKIYEKPATRTFTILSPEVNILFRQYFGDFAAMVMSTRREHFSQVGINPESMEWSDLINSLLRVNTKGFAGDYSKFDGIGSPAIYHSIVNVVNAWYDDGEVNARARHSLISSIVHRDGICGDLILRYSQGMPSGFAMTVIFNSFVNYYFMALAWMSTVGSSLLSPQGSCKDFDTYCKIVAYGDDNVVSVHEEFLDVYNLQTVAAYLSHFGVTYTDGDKNPIHMSKPYEDITKMSFLKRGFERVESSGFLWKAPLDKTSIEERLNWIRDCPTPVEALEQNIESALHEAAIHGRDYFDDLVQRLNSALKRVMLPPTDISFEECQARWWASVTGDALRAADYSSLVRRASSGHVEFNKKYRDMFRQQDLPLKEILMKSKPVALLDLEV

SEQ ID NO: 136 - protein, 2432 aa, synthetic construct
MAATRVSRSVLAAVAHSAAHRTYHTVLDCYDRLYLNTNPHLSYPLPKNSSFPCPFCQYDEQNEVLSPESLRGEGAEPCWKCSQDKPRRKYNTTPPEDWLYDSDVQSWFYPETYYSDLQQKFFDKLALLSLPGAYQAKTPEERALAGALTQLLNFPSTPPLTLPTTNLQRQGNSVTNIYGNGNNVTTDVGANGWAPTVSTGLGDGPVSASADSLPGRSGGASSEKTHTVSGSSNKVGSRFSKWWEPAAARASESATDSAIEGIDAAGKAASKAITRKLDRPAAPSSTANPQPSLIALNPSATQSGNASILTGSTAPSLLAYPTATPVPLPNPDEPSQPGPSGDRTWLLDTVTWSQEFTRGWNIAGSNGMQWTGLESLIFPVSTDTNWTSTSSPTAYPLPFSFVRAYPDSSWAAMYNTHSMWNCGWRVQVTVNGSQFHAGALILYMVPEATTHAIQTARDNAGFVFPYVILNLYESNTATIEVPYISPTPNTSSGLHAPWTFYLQVLSPLNPPPSLPTSLSCSIYVTPVDSSFHGLRYLAPQHWKTRAVPGAGTFGSAVAGQELPLCGVRAYYPPNAYIPAQVRDWLEFAHRPGLMATVPWTMADEPAERLGIFPVSPSAIAGTGAPISYVISLFSQWRGELAAHLLFTGSAQHYGRLVVCYTPAAPQPPSTMQEAMRGTYTVWDVNAASTLEFTIPFISNSYWKTVDVNNPDALLSTTGYVSIWVQNPLVGPHTAPASALVQAFISAGESFNVRLMQNPALTSQTLTEDLDAPQDTGNIENGAADNSPQPRTTFDYTGNPLPPDTKLENFFSFYRLLPMGGSGAPSLSFPADEGTIIPLNPINWLKGADVSGIAAMLSCFTYIAADLRITLRFSNPNDNPATMLVAFAPPGATIPLKPTRQMLSNFYMAEVPVSAATSTMVSFSIPYTSPLSAIPTSYFGWEDWSGTNFGQLSSGSWGNLMLIPSLSVDSAIPFDFQLSCWVAFGNFKAWVPRPPPPLPPLPTPAANAERTVAVIKQGAASATPDVDPDDRVYIVRAQRPTYVHWAIRKVAPDGSAKQISLSRSGIQALVALEPPEGEPYLEILPSHWTLAELQLGNKWEYSATNNCTHFVSSITGESLPNTGFSLALGIGALTAIAASAAVAVKALPGIRRQGLLTLSADTETNQTLNKITESVNQAAQVVSQFDLSGPANSVSLAASDIREAAHKVASSLNGFTDVIADIKDSLFTRVSDAVESGVATFLTWLVKLFGYLLVLFGSPTPMSISGLLVIICADLAPHAREFFTASGNVLSSLYYWIASKLGLSVTPQECERATLEPQGLKDFNDGALAMRNVEWIGETAWKWAHRLLDWIRGKAKTDPQAKLADVHDEIMLHYSDSILALGSEKLPIDHITKSISRCRELVSIAQEAKSGPHSSFLNQAIKNYTLAISQHRKCQTGPRPEPVVVYLYGPPGTGKSLLASLLAQTLSQRLAGTPDDVYSPSSASCEYFDGYTGQTVHFIDDIGQDPEGRDWANFPNLVSSAPFIVPMASLEEKGTHYTSKVIVVTSNFHEPNERAARSMGALRRRVHLRINVTSNGVPFDPTNALNPIPGTQSKYFTAQTPLTLFQSNTVRLDRDSIWTPTFTNMDELVDAIVTRLDRSTGVSNSLASLIRRQGNRVIDAEPREIPLEYADDLLEAMAHHRPVPCSLGLSQAIANNTPIQQISETFWKYRKPIFTCTTFLAVLGFLCSVIPLARSLWKSKQDTPQEPQAAYSAISHQKPKPKSQKPVPTRHIQRQGISPAVPGISNNVVHVESGNGLNKNVMSGFYIFSRFLLVPTHLREPHHTTLTVGADTYDWATLQTQEFGEITIVHTPTSRQYKDMRRFIGAHPHPTGLLVSQFKAAPLYVRISDNRILDLDFPGVVVCKQAYGYRAATFEGLCGSPLVTDDPSGVKILGLHVAGVAGTSGFSAPIHPILGQITQFATTQQSLIVPTAEVRPGVNVNRMSRLHPSPAYGAFPVKKQPAPLKRNDKRLQEGVDLDTQLFLKHGKGDVTEPWPGLEAAADLYFSTFPTSLPVLTQEQAIHGTPNMEGLDMGQAAGYPWNTLGRSRRSLFDEVEPGVFVPKPELQAEINQTLEDPDYVYSTFLKDELRPTAKVEQGLTRIVEAAPIHAIVAGRMLLGGLIDYMQGRPGEHGSAVGCNPDVHWTSFFYAFSEFSQVYDLDYKCFDATLPSAVFTLVADHLTRITGDPRVGRYIHSIRHSHHIYGNRMYDMIGGNPSGCVATSILNTIINNICVLSALIQHPDFSPSRFHILAYGDDVIYATEPPIHPSFLREFYQKHTPLVVTPANKGQDFPPTSTIYEVTFLKRWFVPDDVRPIYIHPVMDPDTYEQSVMWLRDGDFQDVVTSLCHLAFHSGPKTYAAWCMKVREQCLKSGFAPNFLPYSYLQLRWLNLLAA

SEQ ID NO: 137 - protein, 2370 aa, synthetic construct
MMEGSNGFSSSLAGLSSSRSSLRLLTHLLSLPPPNRDARRHSGWYRSPPTLPVNVYLNEQFDNLCLAALRYPGCKLYPSVYTLFPDVSPFKIPQSIPAFAHLVQRQGLRRQGNPTTNIYGNGNEVTTDVGANGMSLPIAVGDMPTASSSEAPLGSNKGGSSTSPKSTSNGNVVRGSRYSKWWEPAAARALDRALDHAVDATDAVAGAASKGIKAGATKLSNKLAGSQTTALLALPGNIAGGAPSATVNANNTSISSQALLPSVNPYPSTPAVSLPNPDAPTQVGPAADRQWLVDTIPWSETTPPLTVFSGPKALTPGTYPPTIEPNTGVYPLPAALCVSHPESVFTTAYNAHAYFNCGFDVTVVVNASQFHGGSLIVLAMAEGLGDITPADSSTWFNFPHAIINLANSNSATLKLPYIGVTPNTSTEGLHNYWTILFAPLTPLAVPTGSPTSVKVSLFVSPIDSAFYGLRFPIPFPTPQHWKTRAVPGAGSYGSVVAGQEIPLVGYAPAAPPRDYLPGRVRNWLEYAARHSWERNLPWTAADEVGDQLVSYPIQPETLANTQTNTAFVLSLFSQWRGSLQISLIFTGPAQCYGRLLLAYTPPSANPPTTIEEANNGTYDVWDVNGDSTYTFTIPFCSQAYWKTVDIGTSSGLVSNNGYFTIFVMNPLVTPGPSPPSATVAAFLHVADDFDVRLPQCPALGFQSGADGAEVQPAPTSDLSDGNPTTDPAPRDNFDYPHHPVDPSTDLAFYFSQYRWFGLNEDLTPLNVTGGLFYHVSLNPVNFQQNSLLSVLGAFTYVYANLSLNINVSAPLQACTFYIFYAPPGASVPSTQTLAELSFFTHTATPLNLAAPTNITVSIPYASPQSVLCTSFGGFGLQNGGDPGNLHSNTWGTLILYVDLPQSDSVSVSAYISFRDFEAYVPRQTPGVGPIPTSTSIVRVARPTPKPRTVRRQGGTLADLILTPESRCFIVAHTTAPYYSILLVNPDEEYAISMFTHGDESILRYSSRGGTRLAPTAPAFFLCAAASVDTILPYPISQSHLWLSDLTGIPLRAVPPLTLFLSAGAALCAGAQTLIAVAQGGSAPDTPPTPNRALFRRQGLGDLPDAAKGLSAALENVAKVAGDADIATSSQAIASSINSLSNSIDGATTFMQNFFSGLAPKNPTSPLQHLFAKLIKWVTKIIGSLIIICNNPTPSALIGVSLMLCGDLAEDITEFFSNLGNPLAAVFYRCARALGLSPTPQSAAQAAGGRQGVRDYNDIMSALRNTDWFFEKIMSHIKNLLEWLGVLVKDDPRTKLNSQHEKILELYTDSVTASSTPPSELSADAIRSNLDLAKQLLTLSHAANSVTHIQLCTRAITNYSTALSAISLVGTPGTRPEPLVVYLYGPPGTGKSLLASLLASTLAQALSGDPNNYYSPSSPDCKFYDGYSGQPVHYIDDIGQDPDGADWADFVNIVSSAPFIVPMADVNDKGRFYTSRVVIVTSNFPGPNPRSARCVAALERRLHIRLNVTARDGAAFSAAAALKPSEPLAATRYCKFSNPLTQFSMFNLAVDYKSIVLPNTPLSCFDELIDFILGSLRDRASVNSLLSGMVRTDVARQGGNADAPAPSAAPLPSVLPSVPSQDPFVRAVNENRPVSFLSKIWSWRAPIFAASSFLSLIAATLTIVRCLRDLRSTQGAYSGTPVPKPRKKDLPKQPVYSGPVRRQGFDPAVMKIMGNVDSFVTLSGSKPIWTMSCLWIGGRNLIAPSHAFVSDDYEITHIRVGSRTLDVSRVTRVDDGELSLISVPDGPEHKSLIRYIRSASPKSGILASKFSDTPVFVSFWNGKPHSTPLPGVVDEKDSFTYRCSSFQGLCGSPMIATDPGGLGILGIHVAGVAGYNGFSARLTPERVQAFLSNLATPQSVLHFHPPMGPPAHVSRRSRLHPSPAFGAFPITKEPAALSRKDPRLPEGTDLDAITLAKHDKGDIATPWPCMEEAADWYFSQLPDSLPVLSQEDAIRGLDHMDAIDLSQSPGYPWTTQGRSRRSLFDEDGNPVPELQKAIDSVWDGGSYIYQSFLKDELRPTAKARAGKTRIVEAAPIQAIVVGRRLLGSLINHLQGNPLQYGSAVGCNPDIHWTQIFHSLTPFSNVWSIDYSCFDATIPSVLLSAIASRIASRSDQPGRVLDYLSYTTTSYHVYDSLWYTMVGGNPSGCVGTSILNTIANNIAIISAMMYCNKFDPRDPPVLYCYGDDLIWGSNQDFHPRELQAFYQKFTNFVVTPADKASDFPDSSSIYDITFLKRYFVPDDIHPHLIHPVMDEATLTNSIMWLRGGEFEEVLRSLETLAFHSGPNNYSTWCEKIKAKIRENGCDATFTPYSVLQRGWVSTCMTGPYPLTG

SEQ ID NO: 138 - protein, 2374 aa, synthetic construct
MEGSNGFSSSLAGLSSSRSSLRLLTHFLSLPTLPVNIYLNARRHSGWYRSPPTLPVNIYLNEQFDNLCLAALRYPGHKLYPSVHTLFPDVSPLKIPHSVPAFAHLVQRQGLRRQGNSITNIYGNGNDVTTDVGANGMSLPIAVGDMPTASTSEAPLGSNKGGSSTSPKSTSNGNVVRGSRYSKWWEPAAARALDRALDHAVDATDAVAGAASKGIKAGAAKLSNKLSGSQTTALLALPGNIAGGAPSATVNANNTSISSQALLPSVNPYPSTPAVSLPNPDAPTQVGPAADRQWLVDTLSWSETIAPLTVFSGPKALTPGVYPPTIEPNTGVYPLPAALCVSHPESVFSTAYNAHAYFNCGFDVTVVVNASQFHGGSLIVLAMAEGLGDITPADSSTWFNFPHTIINLANSNAATLKLPYIGVTPNTSTEGLHNYWTILFAPLTPLAVPTGSPTTVKVSLFVSPIDSAFYGLRFPVPFPAPQHWKTRAVPGAGTYGSVVAGQEIPLVGYAPAAPPRDYLPGRVHNWLEYAARHSWERNLTWTSADEVGDQLVSYPIQPEALANTQTNTAFVLSLFSQWRGSLQISLIFTGPAQCYGRLLLAYTPPSANPPTTIDEANNGTYDVWDVNGDSTYTFTIPFCSQAYWKTVDIGTSSGLVSNNGYFTVFVMNPLVTPGPSPPSATVAAFLHVADDFDVRLPQCPALGFQSGADGAEVQPAPTSDLSDGNPTTDPAPRDNFDYPHHPVDPSTDLAFYFSQYRWFGLNESLTPLDATGGLFYHISLNPINFQQSSLLSVLGAFTYVYANLSLNINVSAPSQPCTFYVFYAPPGASVPSVQTLAELSFFTHTATPLNLAAPTNITVSIPYSSPQSVLCTSFGGFGLQNGGDAGNLHSNTWGTLILYVDLPQSDSVSVSAYISFRDFEAYVPRQTPGVGPVPTSTSIVRVARPTPKPRTARRQGGTLADLILSPESRCFIVAHTTAPFYSILLVNPDEEYAISMFSHGDESILQYSSRSGTRLTPTAPAFFLCAAASVDTVLPYSISQSHLWLTDLTGIPLRAVPPLTLFLSAGAALCAGAQTLIAVAQGGSTPETPPTPNRALLRRQGLGDLPDAAKGLSAALESVARVAGDANIATSSQAIATSINSLSNSIDGATSFMQNFFSGLAPRNPTSPLQHLFAKLIKWVTKIIGSLIIICNNPTPSALIGVSLMLCGDLAEDITEFFSNLGNPLAAVFYRCARALGLSPTPQSAAQAAGGRQGVRDYNDIMSALRNTDWFFEKIMTHIKNLLEWLGVLVKDDPRTKLNGQHEKILELYTDSVTASSTPPSELSADAIRSNLDLAKQLLTLSHAANSVTHIQLCTRAITNYSTALSAISLVGTPGTRPEPLVVYLYGPPGTGKSLLASLLASTLAQALSGDPNNYYSPSSPDCKFYDGYSGQPVHYIDDIGQDPDGADWADFVNIVSSAPFIVPMADVNDKGRFYTSRVVIVTSNFPGPNPRSARCVAALERRLHIRLNVTARDGVAFSAAAALQPSNPPSATRYCKFANPLTQFSMFNLAVDYKSVVLPNTPLTCFDELVDFVLSSLRDRASVNSLLSGMVRTDVTRQGGNADAPAPSAAPLPSVIPSVPSQDPFTRAVNENRPVSFLSKIWSWRAPIFAASSFLSLIAATLTIVRCLRDLRSTQGAYSGTPVPKPRKKDLPKQPVYSGPVRRQGFDPAVMKIMGNVDSFVTLSGTKPIWTMSCLWIGGRNLIAPSHAFVSDEYEITHIRVGSRTLDVSRVTRVDDGELSLLSVPDGPEHKSLIRYIRSASPKSGILASKFSDTPVFVSFWNGKSHSTPLPGVVDEKDSFTYRCSSFQGLCGSPMIATDPGGLGILGIHVAGVAGYNGFSARLTPERVQAFLSHLATPQSVLYFHPPMGPPAHVSRRSRLHPIPPAFGAFPITKEPAALSRKDPRLPEGTDLDAITLAKHDKGDIATPWPCMEEAADWYFSQLPDNLPVLSQEDAIRGLDHMDAIDLSQSPGYPWTTQGRSRRSLFDEDGNPLPELQEAIDSVWDGGSYIYQSFLKDELRPTAKARAGKTRIVEAAPIQAIVVGRRLLGSLINHLQGNPLQHGSAVGCNPDIHWTQIFHSLTSFSNVWSIDYSCFDATIPSVLLSAIASRIAARSDQPGRVLDYLSYTTTSYHVYDSLWYTMIGGNPSGCVGTSILNTIANNIAVISAMMYCNKFDPRDPPVLYCYGDDLIWGSNQDFHPRELQAFYQKFTNFVVTPADKASDFPDSSSIFDITFLKRYFVPDDIHPHLIHPVMDEQTLTNSIMWLRGGEFEEVLRSLETLAFHSGPKNYSAWCEKIKAKIRENGCDATFTPYSVLQRGWVSTCMTGPYPLTG

SEQ ID NO: 139 - protein, 2180 aa, synthetic construct
METIKSIADMATGVVSSVDSTINAVNEKVESVGNEIGGNLLTKVADDASNILGPNCFATTAEPENKNVVQATTTVNTTNLTQHPSAPTMPFSPDFSNVDNFHSMAYDITTGDKNPSKLVRLETHEWTPSWARGYQITHVELPKVFWDHQDKPAYGQSRYFAAVRCGFHFQVQVNVNQGTAGSALVVYEPKPVVTYDSKLEFGAFTNLPHVLMNLAETTQADLCIPYVADTNYVKTDSSDLGQLKVYVWTPLSIPTGSANQVDVTILGSLLQLDFQNPRVFAQDVNIYDNAPNGKKKNWKKIMTMSTKYKWTRTKIDIAEGPGSMNMANVLCTTGAQSVALVGERAFYDPRTAGSKSRFDDLVKIAQLFSVMADSTTPSENHGVDAKGYFKWSATTAPQSIVHRNIVYLRLFPNLNVFVNSYSYFRGSLVLRLSVYASTFNRGRLRMGFFPNATTDSTSTLDNAIYTICDIGSDNSFEITIPYSFSTWMRKTNGHPIGLFQIEVLNRLTYNSSSPSEVYCIVQGKMGQDARFFCPTGSVVTFQNSWGSQMDLTDPLCIEDDTENCKQTMSPNELGLTSAQDDGPLGQEKPNYFLNFRSMNVDIFTVSHTKVDNLFGRAWFFMEHTFTNEGQWRVPLEFPKQGHGSLSLLFAYFTGELNIHVLFLSERGFLRVAHTYDTSNDRVNFLSSNGVITVPAGEQMTLSAPYYSNKPLRTVRDNNSLGYLMCKPFLTGTSTGKIEVYLSLRCPNFFFPLPAPKVTSSRALRGDMANLTNQSPYGQQPQNRMMKLAYLDRGFYKHYGIIVGDHVYQLDSDDIFKTALTGKAKFTKTKLTSDWVIEEECELDYFRIKYLESAVDSEHIFSVDKNCETIAKDIFGTHTLSQHQAIGLVGTILLTAGLMSTIKTPVNAVTIKEFFNHAIDGDEQGLSLLVQKCTTFFSSAATEILDNDLVKFIVKILVRILCYMVLYCHKPNILTTACLSTLLIMDVTSSSVLSPSCKALMQCLMDGDVKKLAEIVAESMSNTDDDEVKEQICDTVKYTKTILSNQGPFKGFNEVSTAFRHIDWWIHTLLKIKDMVLSVFKPSIESKAIQWLERNKEHVCSILDYASDIIVESKDQSKMKTQDFYQRYSDCLAKFKPIMAICFRSCHNSISNTVYRLFQELARIPNRISTNNDLIRIEPIGIWIQGEPGQGKSFLTHTLSRQLQKSCKLNGVFTNPTASEFMDGYDNQDIHLIDDLGQTRKEKDIEMLCNCISSVPFIVPMAHLEEKGKFYTSKLVVATTNKSDFSSTVLQDSGALKRRFPYIMHIRAAKAYSKAGKLNVSQAMATMSTGECWEVSKNGRDWETLKLKDLVDKITIDYNERVKNYNAWKQQLENQTLDDLDDAVSYIKHNFPDAIPYVDEYLNIEMSTLIEQMEAFIEPKPSVFKCFANKIGSKISKASREVVDWFSDKIKSMLSFVERNKAWLTVVSAVTSAISILLLVTKIFKKEESKDERAYNPTLPVAKPKGTFPVSQREFKNEAPYDGQLEHIISQMAYITGSTTGHMTHCAGYQHDEIILHGHSIKYLEQEDELTLHYKNKVFPIEQPSVTQVTLGGKPMDLAILKCKLPFRFKKNSKYYTNKIGTESMLIWMTEQGIITKEVQRVHHSGGIKTREGTESTKTISYTVKSCKGMCGGLLISKVEGNFKILGMHIAGNGEMGVAIPFNFLKNDMSDQGIVTEITPIQPMYINTKTQIHKSPVYGAVEVKMGPAVLSKSDTRLEEPVECLIKKSASKYRVNKFQVNNELWQGVKACVKSKFREIFGMNGIVDMKTAILGTSHVNSMDLSTSAGYSFVKSGYKKKDLICLEPFSVAPLLERLVQDKFHNLLKGNQITTTFNTCLKDELRKLDKIASGKTRCIEACEVDYCIVYRMIMMEIYDKIYQTPCYYSGLAVGINPYKDWHFMINALNDYNYEMDYSQYDGSLSSMLLWEAVEVLAYCHDSPDLVMQLHKPVIDSDHVVFNERWLIHGGMPSGSPCTTVLNSLCNLMMCIYTTNLISPGIDCLPIVYGDDVILSLDKEIEPEKLQSIMADSFGAEVTGSRKDEPPSLKPRMEVEFLKRKPGYFPESTFIVGKLDTENMIQHLMWMKNFSTFKQQLQSYLMELCLHGKDTYQHYIKILEPYLQEWNITVDDYDVVITKLMPMVFD

SEQ ID NO: 140 - protein, 2188 aa, synthetic construct
METIKSIADMATGFTNTIDSTVNAVTEGVSKIGNDSGGEILTKVADDASNLLGPNCVASTSQPENKDVVQATTTVNTLTNLTQHPSAPTMPFTPDFSNVDVFHSMAYDITTGDKNPSKLIRLDTTTWQHTWPRQHLINDVELPKAFWDKNSKPAYGQSRYFAAVRCGFHFQVQINVNQGTAGCALVVYEPKPIVTHGGHLEFGSYTNLPHVLMNLAETTQADLCIPYVSDTNYVKTDSSDLGRLRVYVWTPLTIPSSATNDVDVTVLGSLLQLDFQNPRTYDTDVNIYDNSPLDTKTKYGKLRFSKKILSMSTKYKWTRNKIDIAEGPGSMNMANVLSTTGAQSIALVGERAFYDPRTAGSKSRFGDMIHIAQLFSVMSDTTTPSTSSGIDDLGYLDWSATYVPQQVIHRNVVKLNQFSNLKPFVNAYTYFRGSLVLRMSVYASTFNRGRLRMGFFPNFTTNTTSEMDNAIYTICDIGSDNSFEITIPYTFSTWMRKTNGRPIGLFQVEVLNRLTYNSSCPNKVHCIVQGRLGNDARFYCPTGSLVEFQNSWGSQMDLTDPLCVEDDEAEDCKQTISPDELGLTSAQDDGPLGVEKPNYFLNFRAINVDIFTVSHTKVDNIFGRAWLALEHTFADDGTWRADLNFPTQGHGTLTRLFTYYSGELNVHVLYLSDNGFLRVTHAYDHDNDRSNFLSSNGVITVPAGEQMTLSVPFYSSKPLRTIRETGALGKLICKPLLSGTHSGKIEVYLSLRCPNLFFPSPAPKEKTSRALRGDLANFIDQSPYGQQQQTQMMKLAYLDRGFYKHYGIIVGGYVYQLDSDDIFKTALTGKARFTKTRLTPDWIVEEECELDYFRVKYLESSVNSEHIFSVDSNCETIAKDIFGTHTLSQHQAIGLVGAILLTAGLMSTIKTPVNATTIKEFFNHAIDGDEQGLSLLVQKCTTFFSSAATEILDNDLVKFIVKILVRILCYMVLYCHKPNILTTACLSTLLIMDVTSSSVLSPSCKALMQCLMDGDVKKLAEVVAESMSNTDDDEIKEQICDTVKYTKTILSNQGPFKGFNEVSTAFRHVDWWIHTLLKIKDMVLSVFKPSIESKAIQWLERNKEHVCSILDYASDIIVESKDQTKMKTQEFYQRYSDCLAKFKPIMAICFRSCHNSISNTVYRLFQELARIPNRISTQNDLIRVEPIGVWIQGEPGQGKSFLTHTLSRQLQKSCKLNGVYTNPTASEFMDGYDNQDIHLIDDLGQTRKEKDIEMLCNCISSVPFIVPMAHLEEKGKFYTSKLVIATTNKSDFSSTVLQDSGALKRRFPYIMHIRAAKAYSKSGKLNVSQAMSTMSTGECWEVSKNGRDWETLKLKDLVQKITEDYQERQKNYNAWKQQLENQTLDDLDDAVSYIKHNFPDAIPYIDEYLNIEMSTLIEQMEAFIEPRPSVFKCFAVKLPHKPGKQPRKLWAGSAGKIKSMLSFIERNKAWLTVVSAVTSAISILLLVTKIFKKEESKDERAYNPTLPITKPKGTFPVSQREFKNEAPYDGQLEHIISQMAYITGSTTGHLTHCAGYQHDEIILHGHSIKYLEQEEDLTLHYKNKVFPIENPSVTQVTLGGKPMDLAILKCKLPFRFKKNSKYYTNKIGTESMLIWMTEQGIITKEVQRVHHSGGIKTREGTESTKTISYTVKSCKGMCGGLLISKVEGNFKILGMHIAGNGEMGVAIPFNFLKNDMSDQGIITEVTPIQPMYINTKSQIHKSPVYGAVEVKMGPAVLSKSDTRLEEPVDCLIKKSASKYRVNKFQVNNELWQGVKACVKSKFREIFGVNGIVDMKTAILGTSHVNSMDLSTSAGYSLVKSGYKKKDLICLEPFSVSPMLEKLVQDKFHNLLKGNQITTIFNTCLKDELRKLDKIAAGKTRCIEACEVDYCIVYRMIMMEIYDKIYQTPCYYSGLAVGINPYKDWHFMINALNDYNYEMDYSQYDGSLSSMLLWEAVEVLAYCHDSPDLVMQLHKPVIDSDHVVFNERWLIHGGMPSGSPCTTVLNSLCNLMMCIYTTNLISPGVDCLPIVYGDDVILSLDREIEPERLQSIMADSFGAEVTGSRKDEPPSLKPRMEVEFLKRKPGYFPESTFIVGKLDTENMIQHLMWMKNFSTFKQQLQSYLMELCLHGKDIYQRYIKILDPYLKEWNIVVDDYDVVIAKLMPMVFD

SEQ ID NO: 141 - protein, 2179 aa, synthetic construct
METIKSIADMATGVTKTIDATINSVNEIITNTDNASGGDILTKVADDASNILGPNCYATTSEPENKDVVQATTTVNTTNLTQHPSAPTLPFTPDFSNVDTFHSMAYDTTTGSKNPNKLVRLTTHAWASTLQRGHQIDHVNLPVDFWDEQRKPAYGHAKYFAAVRCGFHFQVQVNVNQGTAGSALVVYEPKPVVDYDKDLEFGAFTNLPHVLMNLAETTQADLCIPYVADTNYVKTDSSDLGQLKVYVWTPLSIPSGSSNQVDVTILGSLLQLDFQNPRVYGQNVDIYDTAPSKPIPLRKTKYLTMSTKYKWTRNKVDIAEGPGSMNMANVLSTTAAQSVALVGERAFYDPRTAGSKSRFDDLVKISQLFSVMADSTTPSANHGIDQKGYFKWSANSDPQAIVHRNLVHLNLFPNLKVFENSYSYFRGSLIIRLSVYASTFNRGRLNGFFPNSSTDETSEIDNAIYTICDIGSDNSFEITIPYSFSTWMRKTHGKPIGLFQIEVLNRLTYNYSSPNEVYCIVQGKMGQDAKFFCPTGSLVTFQNSWGSQMDLTDPLCIEDSVEDCKQTITPTELGLTSAQDDGPLGNDKPNYFLNFKSMNVDIFTVSHTKVDNIFGRAWFAHVHDFTNDGLWRQGLEFPKEGHGALSLLFAYFTGELNIHVLFLSDRGFLRVGHTYDTETNRTNFLSSSGIITVPAGEQMTLSVPSYSNKPLRTVRSSNALGYLLCKPLLTGTSSGRIEIFLSLRCPNFFFPLPAPKPATRKYRGDLATWSDQSPYGRQGKKQLMKLAYLDRGFYKHYGIVVGDDVYQLDSDDIFKTALTGKAKFTKTRLTPDWVVEEECELDYFRIKYLESSVNSEHIFSVDNNCETIAKDIFGSHSLSQHQQIGLIGTILLTAGLMSTIKTPVNPTTIKEFFNHAIEGDEQGLSLLVQKCTTFFSSAATELLDNDLVKFIIKILVRILCYMVLYCHKPNILTTACLSTLLVMDVTSSSVLSPSCKALMQCLMDGDVKKLAEVVAESMSNTDDDEIKEQICDTVKYTKQILSNQGPFKGFNEISTAFRHIDWWIQTLLKIKDMVLSVFKPSVEKRAVEWLERNKEHVCSILDYASDIIVKSKDQTKMKTQEFYQRYNDCLSKFKPIMAMCFRSCHNSISNTVYRLFQELARIPNRMATQNDLIRVEPIGIWIQGEPGQGKSFLTHTLSKQLQKTCGLQGIYTNPTASEFMDGYDNQDIHLIDDLGQTRKERDIEMLCNCISSDPDIVPMAHLEEKGKFYTSKLVIATTNKPDFSSTVLLDSGALRRRFPYIMHIRAAKHYSKSGKLNVSQAMPHMSTGECWEVSKNGRDWETLKLKELIDKITVDYKERIANYNTWKKQLEDQTLDDLDDAVSYIKHNYPDAIPYIDEYLNIEMSTLIEQMEAFIEPKPSVFKCFASRVGDKIKEASREVVKWFSDKLKSMLNFVERNKAWLTVVSAVTSAIGILLLVTKIFKKEESKDERAYNPTLPVAKPKGTFPVSQREFKNEAPYDGQLEHIISQMAYITGSTTGHITHCAGYQHDEIILHGHSIKYLEQEEELTLHYKNKVFPIEQPSVTQVTLGGKPMDLAIVKCKLPFRFKKNSKYYTNKIGTESMLIWMTEQGIITKEVQRVHHSGGIKTREGTESTKTISYTVKSCKGMCGGLLISKVEGNFKILGMHIAGNGEMGVAIPFNFLKNDMSDQGIVTEVTPIQPMYINTKSQIHKSPVYGAVEVKMGPAVLSKSDTRLEEPVDCLVKKSASKYRVNKFQVNNELWQGVKACVKSKFREIFGVNGIVDMKTAILGTSHVNSMDLSTSAGYSFVKSGYKKKDLICLEPFSVSPMLEKLVQEKFHNLLKGNQITTIFNTCLKDELRKLDKIATGKTRCIEACEIDYCIVYRMIMMEIYDKIYQTPCYYSGLAVGINPYRDWHFMINALNDYNYEMDYSQYDGSLSSMLLWEAVQVLAYCHDSPDLVMQLHKPVIDSDHVVFNERWLIHGGMPSGSPCTTVLNSLCNLMMCIYTTNLISPGIDCLPIVYGDDVILSLDKEIEPERLQSIMAESFGAEVTGSRKDEPPSLKPRMEVEFLKRKPGYFPESTFIVGKLDTENMIQHLMWMKNFSTFKQQLQSYLMELCLHGKDTYQHYVKILNPYLKEWNIPVDDYEVVIGKLVPMVFD

SEQ ID NO: 142 - protein, 2253 aa, synthetic construct
MAASKMNPVGNLLSTVSSTVGSLLQNPSVEEKEMDSDRVAASTTTNAGNLVQASVAPTMPVKPDFKNTDDFLSMSYRSTTAPTNPTKMVHLAHGTWTTNQHRQALVASITLPQAFWPNQDFPAWGQSRYFAAVRCGFHIQVQLNVNIGSAGCLIAAYMPKTAHDHMNTYTFGSYTNLPHVLMNAATTSQADLYIPYVFNHNYARTDSDDLGGIYIWVWSALTVPSGSPTTVDVTIFGSLLDLDFQCPRPPGADTVIYTQGKRTVRKTKTSKFKWVRNKIDIAEGPGAMNIANVLSTTGGQTIALVGERAFYDPRTAGAAVRCKDLMEIARMPSVFLGESTEPDGRRGYFTWSHTISPVNWVFDDHIYLENMPNLRLFSSCYNYWRGSFVIKLTVYASTFNKGRLRMAFFPNREGAYTQDEAQNAIFVVCDIGLNNTFEMTIPYTWGNWMRPTRGNSLGHLRIDVLNRLTYNSSSPNAVNCILQIKMGDDAMFMVPTTSNLVWQGLHSWGSEMDLVDSLDNPDEIQDNEEIQTQNVEAAQGEEAATEVGLRATENDGSLSEQLNMSQPMFLNFKKHKVNIYAASHTKVDHIFGRAWAVGVFNTETAAIQKFDLHFPTSTHGALSRFFCFWTGELNIHILNVSTTNAFLKVAHTWFGTDSGIARTATLESNGTMIIPPNEQMTLCVPYYSEVPLRCVKGSDRNSAGLGSLFTQAVGRTISNRVQIFVSFRCPNFFFPLPAPREATSRSILERVDEANAEELEAVLEARTPDAPLRLKFNPEDPLKQLREAAKAYFNIMHSDEMDFAGGKFLNQCGDVETNPGPDIELVYKNRGFYKHYGVRFGGHIYHLNSQDILSTAITGKSDFIKEEDDGKWVHAMTAPLDYFTEKYINSMVGSKHIFSATSNCETIARDLFPGRKEITQSKALGIIGVILLSASLLSLLAVPWDYSSLQTVYNQSIEGDASGLTLLSQRCMTFFSNTMCETFNNDLVKFIIKILVRLLCYIVLYCHAPNMLTTMCLGTLLVLDITTCEILSANTKALFQALVDGDVKSLVWKIAENMQFAQSKDEQAEDMAATFNFASDMVNFVPMEQMRQEGWREFNDVSMSFRHVEWWLTMFKKVYNVLKSIFAPSIEQKAVDWIDRNQEYIADVLDHASNIIIKMKDPKEQRRASTISEYFEVLKQLKPIVSLCMKVAPSTKFSSQVFRIYSEMMRVNVRVPANTDLTRLEPIGIWVSSEPGQGKSFFTHMLSTCLLKSCNLEGIYTNPTGSEFMDGYIGQDIHIIDDAGQNREEKDLALLCQCISSVPFTVPMADLTEKGTFYTSKIVIATTNKFDFTSMVLTDPAALERRFPFHLRIRAVASYSRNNKLDVARSMAAMADGSCWEYSTDGGRAWKTLSMDELVKQITAVYTQRSDALMVWKRKLNTIRNEMSPGSSTGRIFEPLEETLCALERRFGQLADSLKDNYHKTADELIEAIEDMMAPSQSPFACFAESYRPTIKYTASDKVKSWVKNHMNRWKEFVMRNKGWFTLFSVLSSFLSILTLVYLHYKKEKKEEERQERAYNPQTAISKKGGKPKLSLVKTTNFVNEAPYMQDLEHCFAQTAYISSPETQDIIHCAALSEDTILVYGHSQFYFNRYEDLRLHFKGAIFPIEGGKISQVTVNGQPMDLILVKIDKLPITFKNYTKYYTTEVGKETLLIWNSEKGRLAMPVQCVAPAGPVETMEGTITHKTYSYKVASKKGMCGGLLVTRVHGTFKVLGMHIAGNGQVARAAAVHFISNGAAGFMDQGVVVAKEKLQKPIYLPSKTALNPSPLNGVVPVKMEPAVLSPHDTRLEVIMPSVVKTAAAKYRVNIFNPDFEIWERVVDELKSKFRTKLGIHKHVSFQKAVQGFSSLSSLDLSTSPGQKYVEKGMKKRDLLSTEPFWMHPQLEGDVKDILGAVYSGKKPHTFFAAHLKDELRKKEKIAQGKTRCIEACSIDYVIAYRVVMSSLYEAIYQTPAQELGLAVGMNPWTDWDPMINVLQPYNYGLDYSSYDGSLSEQLMRYGVEILAYCHEQPEAVMILHEPVINSQHLVMDEIWHVNGGMPSGAPCTTVLNSICNLLVCTYLAYEQSLDIEVLPIVYGDDVIFSVSSPLDAEYLVQSAAQNFGMEVTSSDKSGPPKLLKMDEIEFLKRTTKFFPGSTYKVGALSLDTMEQHIMWMKNLETFPEQLVSFENELVLHGKEIYDDYKNRFNPILNQWRVCMQDYEVALHRMLRYVFD

SEQ ID NO: 143 - protein, 2104 aa, synthetic construct
MLIEAFINSLLQNLGIMMSFRDLVASPWILLVIAIPLCAFASSASMVREMLFRHKITENILKGTGVEELFNPFGIIIKYFLYFAILYAFIKYIRNNINVITEKVNFIRRVVSNPTGTTGRRGVLGRCVEQIVEYPTFFITMVYELQQIKNKKDLISKITMISSILKLPLGIWESTVGRMLDRPAIEGTEEMLEDVLPIVAMGLTITKTQIGDVPVESFLVNLDRNQKACENIIKRMQPLMIKMGMMKDSSYDTILQVAKEVNELSEAETWMKTTLKLNPNEFLQTQGAVRVGEIREKVATLRNKLNTLQTKELRSDKVVTECQKHLASLEVLLIEVKVLENSNQTRVKPVGVTIQGEKQIGKTNLVAILSRKICEYVQEHGDISFRNATKWTTWSRQCRDEFDTGYTGQEITYVDDAFQQKDNKDHLMWFTFISNTAVGTNQADLKQKGLPYRSKLVFTTCNKSPDKSVTIEDIEALHARFPHTICLRRNKNKMPKRGAIDESYDWVDFYYGPMSKAVSAIGSNTTSTLKTMSLSEIVKIIGDDLIIQNNFYNSTIKDVGITGQEQMDGAQLERRQRMRELRDHLLRIRSGDENMPFLDETFELNSRPIQTDEKFIPLKDNLDEEVMYGGISDQLLTRFDNIIERSLEGYDVESRELGVEPLTTLNHVRSNMLSYRAWNLINSMCINKTETFSAWLGRYITECVEGVAENLVTTKVKIRINPFTGLQLIAAKRMLQENKLIDMDEIPSTSANSYETVYDQIKNFVNDELSLMETDVVDLALAKISLSQIRSNIKRSTWLDVNDWILALKHKISGKSFAKHMDLYPSSLDSFLLTLKDWEVEDRIKFNSIYKQKVLFVQSRFSLYCWSPFISRGTRFVKVTSRFRELVDKLETGILFHEIKSVTNGIRWLGGAGNNGHVGERVRVIAHTAQFPKKSYPQNGFPINEELHREWIQLVINSDYKYHSLIGEEKVNILWNLIRLQPQREVENFKVYLEDLQASPPKTGTICAKVVNDIKAEVTSSYRQFNNYYTRLTKDGMHTLLSMLSRIGVPISDYWNDLLVDKAPAITAVTVGAITSLAIITIVKTFQYAIAGEEQSKGEKRAKQKNIATTKLQKLKFTLGKEQAEGDSIEHVKEFDGDVKFETIEKLFDHIDEHPNLNIVGLNLVAPENPIAIYAAREESYDFSFSEPRPPQWKKVVTFKEDSKRIISLQLRGEDTEDNILDEIEHAIKVSHGMPYAEWIFNGWFKKESNDNILYCVELDLVTAKTQSGPVGWTRAQTKNLKDLEIQLNKGKPIDVKSVVLGAPQASTQATDTMDVLVNKHLVKVHCLSYENLNNLALNGTQVFALASDNILIVPAHAARQNKWIRFSRATQTGHYGVAKVDERRIDFTRDIAIAIILTRAEAEQKLCELGYSIQLTNISKEKFHFPLITKYLLTADQSEVEWMNCTTLHYFAKNKTVGLGRTTSFQVSEFLCGNEYISKKLVACAQGLQSSVELSRLGDCGSPIVLASGKKAGKLIGFHGYHSPNLQTWYGAMLTVEDLGIINGVEEHFDDPWAKLITQGLPVDLPIGPEVEYVGNLIRPSLPVTNDSLDHWHKSPFADQFEEQLAPGRLNPYDSYIEGDLPTNLEGRKSLILGPNSEMAKTLPELDQGILDWIVDQLVVEQVATFKAENLLTKVSDDIDEMLDYALNGNVDNTYVRGMEVNKASGLPWSLSGSPKKSDFIDVDEATGVRSFKVNANGDALKNRVILKLQQAKMGNRILSFSSSKLKDQPIKIAQAKSGRTRVFHCIPVDLILFSGALYGPYKEAYTKAGLKCYHAVGIDPKSVGWQQLATYMTKHPNYFDADYKNYDKYLHRQVFKAVRKIQRSVIQQVCPDKWDKARAVEELDAIDTYVVDYQTVYKTNRGNKSGSYTTTIDNCLANDIYGLYAWVKTTGLRSLWDYRQNVSSVAFGDDIIKSVSDEYKDKYNYCTYRDVLNATGHIMTPGSKDGEEKPFTSFENLQFLKRGFKLENGMVLAPLLQRSIEGPFVWTDIREDQITVWVNLVQEQLIEAALWGEEYYNELCQKLKCGTNRTLNGALAVLLNTSWEVTFQKFCNRYYGIKRGDL

SEQ ID NO: 144 - protein, 2227 aa, synthetic construct
MNMSKQGIFQTVGSGLDHILSLADIEEEQMIQSVDRTAVTGASYFTSVDQSSVHTAEVGSHQIEPLKTSVDKPGSKKTQGEKFFLIHSADWLTTHALFHEVAKLDVVKLLYNEQFAVQGLLRYHTYARFGIEIQVQINPTPFQQGGLICAMVPGDQSYGSIASLTVYPHGLLNCNINNVVRIKVPFIYTRGAYHFKDPQYPVWELTIRVWSELNIGTGTSAYTSLNVLARFTDLELHGLTPLSTQMMRNEFRVSTTENVVNLSNYEDARAKMSFALDPEDWKSDPSQGGGIKITHFTTWTSIPTLAAQFPFNASDSVGQQIKVIPVDPYFFQMTNTNPDQKCITALASICQMFCFWRGDLVFDFQVFPTKYHSGRLLFCFVPGNELIDVTGITLKQATTAPCAVMDITGVQSTLRFRVPWISDTPYRVNRYTKSAHQKGEYTAIGKLIVYCYNRLTSPSNVASHVRVNVYLSAINLECFAPLYHAMDVTTQVGDDSGGFSTTVSTEQNVPDPQVGITTMRDLKGKANRGKMDVSGVQAPVGAITTIEDPVLAKKVPETFPELKPGESRHTSDHMSIYKFMGRSHFLCTFTFNSNNKEYTFPITLSSTSNPPHGLPSTLRWFFNLFQLYRGPLDLTIIITGATDVDGMAWFTPVGLAVDTPWVEKESALSIDYKTALGAVRFNTRRTGNIQIRLPWYSYLYAVSGALDGLGDKTDSTFGLVSIQIANYNHSDEYLSFSCYLSVTEQSEFYFPRAPLNSNAMLSTESMMSRIAAGDLESSVDDPRSEEDRRFESHIECRKPYKELRLEVGKQRLKYAQEELSNEVLPPPRKMKGLFSQAKISLFYTEEHEIMKFSWRGVTADTRALRRFGFSMAAGRSVWTLEMDAGVLTGRLVRLNDEKWTEMKDDKIVSLIEKFTSNKYWSKVSFPHGMLDLEEIAANSTDFPNMSETDLCFLLHWLNPKKINLADRMLGLSGVQEIKEQGVGLIAECRTFLDSIAGTLKSMMFGFHHSVTVEIINTVLCFVKSGILLYVIQQLNQDEHSHIIGLLRVMNYADIGCSVISCGKVFSKMLETVFNWQMDSRMMELRTQSFSNWLRDICSGITIFKSFKDAIYWLYTKLKDFYEVNYGKKKDILNILKDNQQKIEKAIEEADNFCILQIQDVEKFDQYQKGVDLIQKLRTVHSMAQVDPNLGVHLSPLRDCIARVHQKLKNLGSINQAMVTRCEPVVCYLYGKRGGGKSLTSIALATKICKHYGVEPEKNIYTKPVASDYWDGYSGQLVCIIDDIGQNTTDEDWSDFCQLVSGCPMRLNMASLEEKGRHFSSPFIIATSNWSNPSPKTVYVKEAIDRRLHFKVEVKPASFFKNPHNDMLNVNLAKTNDAIKDMSCVDLIMDGHNISLMDLLSSLVMTVEIRKQNMSEFMELWSQGISDDDNDSAVAEFFQSFPSGEPSNSKLSSFFQSVTNHKWVAVGAAVGILGVLVGGWFVYKHFSRKEEEPIPAEGVYHGVTKPKQVIKLDADPVESQSTLEIAGLVRKNLVQFGVGEKNGCVRWVMNALGVKDDWLLVPSHAYKFEKDYEMMEFYFNRGGTYYSISAGNVVIQSLDVGFQDVVLMKVPTIPKFRDITQHFIKKGDVPRALNRLATLVTTVNGTPMLISEGPLKMEEKATYVHKKNDGTTVDLTVDQAWRGKGEGLPGMCGGALVSSNQSIQNAILGIHVAGGNSILVAKLVTQEMFQNIDKKIESQRIMKVEFTQCSMNVVSKTLFRKSPIHHHIDKTMINFPAVMPFSKAEVDPMAVMLSKYSLPIVEEPEDYKEASIFYQNKIVGKTQLVDDFLDLDMAITGAPGIDAINMDSSPGFPYVQEKLTKRDLIWLDENGLLLGVHPRLAQRILFNTVMMENCSDLDVVFTTCPKDELRPLEKVLESKTRAIDACPLDYTILCRMYWGPAISYFHLNPGFHTGVAIGIDPDRQWDELFKTMIRFGDVGLDLDFSAFDASLSPFMIREAGRIMSELSGTPSHFGTALINTIIYSKHLLYNCCYHVCGSMPSGSPCTALLNSIINNINLYYVFSKIFGKSPVFFCQALRILCYGDDVLIVFSRDVQIDNLDLIGQKIVDEFKKLGMTATSADKNVPQLKPVSELTFLKRSFNLVEDRIRPAISEKTIWSLIAWQRSNAEFEQNLENAQWFAFMHGYEFYQKFYYFVQSCLEKEMIEYRLKSYDWWRMRFYDQCFICDLS

SEQ ID NO: 145 - protein, 2227 aa, synthetic construct
MNMSRQGIFQTVGSGLDHILSLADIEEEQMIQSVDRTAVTGASYFTSVDQSSVHTAEVGSHQVEPLRTSVDKPGSKKTQGEKFFLIHSADWLTTHALFHEVAKLDVVKLLYNEQFAVQGLLRYHTYARFGIEIQVQINPTPFQQGGLICAMVPGDQSYGSIASLTVYPHGLLNCNINNVVRIKVPFIYTRGAYHFKDPQYPVWELTIRVWSELNIGTGTSAYTSLNVLARFTDLELHGLTPLSTQMMRNEFRVSTTENVVNLSNYEDARAKMSFALDQEDWKSDPSQGGGIKITHFTTWTSIPTLAAQFPFNASDSVGQQIKVIPVDPYFFQMTNTNPDQKCITALASICQMFCFWRGDLVFDFQVFPTKYHSGRLLFCFVPGNELIDVSGITLKQATTAPCAVMDITGVQSTLRFRVPWISDTPYRVNRYTKSAHQKGEYTAIGKLIVYCYNRLTSPSNVASHVRVNVYLSAINLECFAPLYHAMDVTTQVGDDSGGFSTTVSTEQNVPDPQVGITTMKDLKGKANRGKMDVSGVQAPVGAITTIEDPVLAKKVPETFPELKPGESRHTSDHMSIYKFMGRSHFLCTFTFNSNNKEYTFPITLSSTSNPPHGLPSTLRWFFNLFQLYRGPLDLTIIIIGATDVDGMAWFTPVGLAVDTPWVEKESALSIDYKTALGAVRFNTRRTGNIQIRLPWYSYLYAVSGALDGLGDKTDSTFGLVSIQIANYNHSDEYLSFSCYLSVTEQSEFYFPRAPLNSNAMLSTESMMSRIAAGDLESSVDDPRSEEDKRFESHIECRKPYKELRLEVGKQRLKYAQEELSNEVLPPPRKKKGLFSQAKISLFYTEEHEIMKFSWRGVTADTRALRRFGFSLAAGRSVWTLEMDAGVLTGRLIRLNDEKWTEMKDDKIVSLIEKFTSNKYWSKVNFPHGMLDLEEIAANSKDFPNMSETDLCFLLHWLNPKKINLADRMLGLSGVQEIKEQGVGLIAECRTFLDSIAGTLKSMMFGFHHSVTVEIINTVLCFVKSGILLYVMQQLNQDEHSHIIGLLRVMNYVDIGCSVISCGKVFSKMLETVFNWQMDSRMMELRTQSFSNWLRDICSGITIFKNFKDAIYWLYTKLNDFYEVNYGKKKDILNILKDNQQKIEKAIEEADKFSILQIQDVEKFEQYQKGVDLIQKLRTVHSMAQVDPNLMVHLSPLRDCIARVHQKLKNLGSINQAMVTRCEPVVCYLYGKRGGGKSLTSIALATKICKHYGVEPEKNIYTKPVASDYWDGYSGQLVCIIDDIGQNTTDEDWSDFCQLVSGCPLRLNMASLEEKGRHFSSPFIIATSNWSNPSPKTVYVKEAIDRRLHFKVEVNPASFSKNPHNDMLNVNLAKTNDAIKDMSCVDLIMDGHNVSLMDLLSSLVMTVEIRKQNMTAFMELWSQGISDDDNDSAMAEFFQSFPSGEPSNSKLSGFFQSVTNHKWVAVGAAVGILGVLVGGWFVYKHFSRKEEEPIPAEGVYHGVTKPKQVIKLDADPVESQSTLEIAGLVRKNLVQFGVGEKNGCVRWVMNALGVKDDWLLVPSHAYKFEKDYEMMEFYFNRGGTYYSISAGNVVIQSLDVGFQDVVLMKVPTIPKFRDITQHFIKKGDVPRALNRLATLVTTVNGTPMLISEGPLKMEEKATYVHKKNDGTTVDLTVDQAWRGKGEGLPGMCGGALVSSNQSIQNAILGIHVAGGNSILVAKLVTQEMFQNIDKKIESQRIMKVEFTQCSMNVVSKTLFRKSPIHHHIDKTMINFPAAMPFSKAEIDPMAMMLSKYSLPIVEEPEDYKEASIFYQNKIVGKTQLVDDFLDLDMAITGAPGIDAINMDSSPGFPYVQERLTKRDLIWLDENGLLLGVHPRLAQRILFNTVMMENCSDLDVVFTTCPKDELRPLEKVLESKTRAIDACPLDYTILCRMYWGPAISYFHLNPGFHTGVAIGIDPDCQWDELFKTMIRFGDVGLDLDFSAFDASLSPFMIREAGRIMSELSGTPSHFGTALMNTIIYSKHLLYNCCYHVCGSMPSGSPCTALLNSIINNVNLYYVFSKIFGKSPVFFCQALKILCYGDDVLIVFSRDVQIDNLDLIGQKIVDEFKKLGMTATSADKNVPQLKPVSELTFLKRSFNLVEDRIRPAISEKTIWSLIAWQRSNAEFEQNLENAQWFAFMHGYEFYQKFYYFVQSCLEKEMIEYRLKSYDWWRMRFYDQCFICDLS

SEQ ID NO: 146 - protein, 2230 aa, synthetic construct
MFMMMNMSKQGIFQTVGSGLDHILSLADVEEEQMIQSVDRTAVTGASYFTSVDQSSVHTAEVGAHQTEPLKTSVDKPGSKKTQGEKFFLIHSADWLTTHALFHEVAKLDVVSLLYNEQFAVQGLLRYHTYARFGIEIQVQINPTPFQQGGLICAMVPGDQGYGSIASLTVYPHGLLNCNINNVVRIKVPFIYTRGAYHFKDPQYPVWELTIRVWSELNIGTGTSAYTSLNVLARFTDLELHGLTPLSTQMMRNEFRVSTTENVVNLSNYEDARAKMSFALDQEDWKTDPSQGGGIKITHFTTWTSIPTLAAQFAFNASASVGQQIKVIPVDPYFYQMTNSNPDQKCITALASVCQMFCFWRGDLVFDFQVFPTKYHSGRLLFCFVPGNELIDVSGITLKQATTAPCAVMDITGVQSTLRFRVPWISDTPYRVNRYTKSAHQKGEYTAIGKLIVYCYNRLTSPSNVASHVRVNVYLSAINLECFAPLYHAMDVTSQTGDDSGGFSTTVSTEQNAPDPQVGITTIKDLKGKANRGKMDVSGIQAPVGAITTIEDPVLAKKVPETFPELRPGESRHTSDHMSIYKFMGRSHFLCTFTFNANNREYTFPITLSSTSNPPHGLPSTLRWFFNLFQLYRGPLDLTIIITGATDVDGMAWFTPVGLAVDTPWVEKQSALTIDYKTALGAIRFNTRRTGNIQIRLPWYSYLYAVSGALDGLGDTTDSTFGLVSIQIANYNHSDEYLSFSCYLSVTEQSEFYFPRAPLNSNAMMVSESMLDRIASGDLESSVDDPRSAEDKRFESHIEQGKPYKELRMEVGKQRLKYAMEELSNEILPPPRKVKGLFSQAKISLFYTEDHEIVKLSWKGLTADTRALRRYGFSLAAGRSVWTLEMEAGVLTGRMIRLNDEKWTEIKDDKIVALVEKFTSNKNWSKVNFPHGMLDLEEIASNSKDFPNMSETDLCFLLHWLNPKKINLADRMLGLSGVQEIKEQGVGLIAECRTFLDSIAGTLKSMMFGFHQSVTVEIINTVLCFVKSGILLYVIQQLNQNEHSHIIGLLQVMNYADIGCSVISCGKIFSKMLETVFNWQMDSRMMALRTQSFSNWLRDICSGITIFKNFKDAIFWLYTKLKDYYDSNYGKKKDVLNVLKENQHRIEKAIEEADQFCVLQIQDVEKSEQYQKGVELIQKLRTVHSLAQVDSSLMSHLSPLRDCIARVHQKLKNLGSINQAMVTRCEPVVCYLYGKRGGGKSLTSIALATKICKHYGVEPEKNIYTKPVASDYWDGYSGQLVCIIDDIGQNTTDEDWSDFCQLVSGCPMRLNMASLEEKGRHFSSPFIIATSNWSNPSPKTVYVKEAIDRRLHYKIEVKPASFYKNAHNDMLNVNLARNNDAIKDMSCVDLLMDGHTVSLSELLNSLVMTVEIRKQNMSEFMKLWSQGVSDDDNDSAVAEFFQSFPSGEPSNSKLSSFFKAVTNHKWVAIGAAVGVLGVLVGGWFVYKHFTKEEPIPTEGVYHGVTKPKQVIKLDADPVDSQSTLEIAGLVRKNLVQFGVGEKNGCVRWVMNALGIKDDWLLVPSHAYKFEKDYQMMEFYFNRGGTYYSISAGNVVIQSLDVGFQDVVLMKVPTIPKFRDITEHFIKKNDVPRALNRLATLVTTVNGTPMLISEGPLKMEEKATYVHKRNDGTTVDLTVDQAWRGKGEGLPGMCGGALISSNQSIQNAILGIHVAGGNSILVAKLVTQEMFQNIEQKAIESQRIMKVEFTQCSMNVVSKTLFKKSPIHHHIDRNMINFPAVMPFSKAEIDPMAVMLSKYSLPIVEEPDDYKMASIYFQNKVMGKTFLVDDFLDIDMAITGAPGIDAINMDSSPGFPYVQEKLTKKDLIWLDENGLLLGVHPRLAQRILYNTVMMENCSDLDVVFTTCPKDELRPLDKVLESKTRAIDACPLDYTILCRIYWGPAISYFQLNPGFHTGVAIGIDPDRHWDELFKTMVRFGDVGLDLDFSSFDASLSPFMIREAGRILSEMSGTPSHFGEALINTIIYSKHLLYNCCYHVYGSMPSGSPCTALLNSIVNNVNLYYVFSKIFRKSPVFFGDALKILCYGDDVLIVFSRNVQIDNLESIGQKIVDEFGKLGMTATSADKSVPKLKPISELTFLKRSFNLVEDRIRPAISEKTIWSLVAWQRSNAEFEQNLENAQWFAFMHGFEFYQKFYHFVQSCLEKEMVEYRLKSYDWWRMKFYDQCFVCDLT

SEQ ID NO: 147 - protein, 2134 aa, synthetic construct
MSKLFSTVGKTVDEVLSVLNDENTESYAGPDRTAVVGGGFLTTVDQSSVSTATMGSLQDVQYRTAVDIPGSRVTQGERFFLIDQREWNSTQSEWQLLGKIDIVKELLDQSYAVDGLLKYHSYARFGLDVIVQINPTSFQAGGLIAALVPYDQVDIESIAAMTTYCHGKLNCNINNVVRMKVPYIYSRGCYNLRNSAYSIWMLVIRVWSQLQLGSGTSTQITVTTLARFVDLELHGLSPLVAQMMRNEFRLSSSSNIVNLANYEDARAKVSLALGQEEFSRDSSSTGGELLHHFSQWTSIPCLAFTFTFPGTVGPGTQIWSTTVDPFSCNLRASSTVHPTNLSSIAGMFCFWRGDIVFEFQVFCTKYHSGRLMFVYVPGDENTKISTLTAKQASSGLTAVFDINGVNSTLVFRCPFISDTPYRVNPTTHKSLWPYATGKLVCYVYNRLNAPASVSPSVSINVYKSAVDLELYAPVYGVSPTNTSVFAQGKEDEGGFSSVPEVEQHVVEDKEPQGPLHVTPFGAVKAMEDPQLARKTPGTFPELAPGKPRHTVDHMDLYKFMGRAHYLWGHKFTKTDMQYTFQIPLSPIKEGFVTGTLRWFLSLFQLYRGSLDITMTFAGKTNVDGIVYFVPEGVAIETEREEQTPLLTLNYKTSVGAIRFNTGQTTNVQFRIPFYTPLEHIATHSKNAMDSVLGAITTQITNYSAQDEYLQVTYYISFNEDSQFSVPRAVPVVSSFTDTSSKTVMNTYWLDDDELVEESSHSSFDEIEEAQCSKCKMDLGDIVSCSGEKAKHFGVYVGDGVVHVDPEGNATNWFMKRKATVKKSKNLDKWCFALSPRIDRTLICETANLMVGREVEYDIFVKNCETYARGIASGDYGTKEGEKWKTLLSAVGVAAMTTTMMAMRHELLDTSLTKLPQKVGEVTNEVRKILEDTSAGVREFKEKVSSILRKTWPGKTSIKIMKWTCRIVKMCVGVGLCYAHGWDSKTVTAVVTMFSMDFLDLVIDGIEIGRMIIDELTTPKAQGLSEINQVLSIAKNAKDVIKMLIEIFCKVIERITGEHGKKIQWAQDKKEEIMNVLERAEKWITTSDDHSEGIECLKLVRSIQSVIRGEESLKELAGELRAVGTHVLNKLGRLDKPNAPILVRAEPTVLYLYGNRGGGKSLASMAIAVKLCKELGISHVEGIYTKPIMSDFWDGYAGQPVVIMDDLGQSTSDEDWTNFCQLVSSCPLRLNMANLEKKGTQFNSPFIIASSNLSHPCPKTVYCTDAIARRLHIKVKVSPKEEFSTHAMLDVAKAKKAGAYCNLDCLDFQKISDLASTPVSVQDIVLEMLHTNVDKQTLMGDIIQYWAQSNPREVFDTMAEGKNSGKYLWLFEKIKTSKWYILGCVGAVLSVSVLGVFAYHMIKNHFRDQQHDQSAYSAAIKPLRVVRLEQSDAQSVVDISNVVHGNLVRVGVGPNEARIHWLYNGLGVYDTYILMPYHGIKDADVDDDLYIERAGTIYSTNMKMVQVLFLESREGDLVLINVPRLPKFRDIRNHFSTEENIRRAEGMPGTLCTLDHERFTLVTESDLKMVEAATYVCEDDKGVRTDISVGRSWKAKACTVAGMCGGALVTSNNKMQNAIVGIHVAGGAHAISRVITKEMIEEMLKTRAQCSRIWKTEFVEEKISVGSKTKYHKSPLYDFCPQEVIKCPTKLFYQGEIDVMQVMLAKYSSPIVSEPLGYATVVEAYTNRMVSFFSEPRQLTYDECINGIEGLDAIDLKTSAGFPYNTLGLRKSDLIINGKMAQRLQQDVEKMEEDLHMNRSIQVVFTTCAKDELRPLSKVMLGKTRAIEACPVSFTILFRRYLGYALAQIQSHPGFHTGIAVGVDPDQDWHCMWYSIVTQCDLVVGLDFSNYDASLSPFMIYHAGRVLGQICGLDPRLVDRIMEPIVNSVHQLGSMRYYVHGSMPSGTPATSVLNSIINVVNICHVLCALEKISVFEVFKLFKILTYGDDVLLCIKKEYLDQKSFPLSSFVQGLEELGLSPTGADKMEVKVTPVHKMSFLKRTFYVDEWSICHPRISEETVYSMLAWKSDNASMKDLIETSIWFMFHHGPRKYVRFCTWLRGVLCRVGIGLYIPTYKELEVRYDRLVKYRFIDDSF

SEQ ID NO: 148 - protein, 2134 aa, synthetic construct
MSKLFSTVGRTVDEVLSVLNDEDTESYAGPDRTAVVGGGFLTTVDQSSVSTATMGSLQDVQYRTAVDIPGSRVTQGERFFLIDQREWNSTQSEWQLLGKIDIVKELLDQSYAVDGLLKYHSYARFGLDVIVQINPTSFQAGGLIAALVPYDQVDIESIVAMTTYCHGKVNCNINYVVRMKVPYIYSRGCYNLRNSAYSIWMLVIRVWSRLQLGSGTSTQITITTLARFVDLELHGLSPLVAQMMRNEFRLSSSSNIVNLANYDDARAKVSLALGQEEFSRDSSSTGGELVHHFSQWTSIPCLAFTFTFPGTVGPGTHIWSTTVDPFSCNLRASSTVHPTNLSSIAGMFCFWRGDIVFEFQVFCTKYHSGRLMFVYVPGDENTKISTLTAKQASTGLTAVFDINGVNSTLVFRCPFISDTPYRVNPTTHKSLWPYATGKLVCYVYNILNAPASVSPSVSINVYKSAADLELYAPVYGVSPTNTSIFAQGKEDEGGFFSVPEVEQHVVEDKEPQGPLHVTPFGAVKAMEDPQLARKTPGTFPELAPGKPRHTVDHMDLYKFMGRAHYLWGHEFTKTDMQYTFQIPLSPIKEGFVTGTLRWFLSLFQLYRGSLDITMTFAGKTNVDGIVYFVPEGVAIETEREEQTPLLTLNYKTSVGAIRFNTGQTTNVQFRIPFYTPLEHIATHSKNAMDSVLGAITTQITNYSAQDEYLQVTYYISFNEDSQFSVPRAVPVVSSFTDTSSKTVMNTYWLDDDELVEESSHSSFDEIEEAQCSKCKMDLGDIVICSGEKAKHFGVYVGDGVVHVDPEGNATNWFMKRKATVKKSKNLDKWCFALSPRIDRTLICETANLMVGREVEYDIFVKNCETYARGIASGDYGTKEGEKWKTLLSAVGVAAMTTTMMAMRHQLLDTSLTKLPQKVGEVTNEVRKILEDTSAGVREFKEKVSSILRKTWPGKTSIKIMKWTFRIVKMCVGVGLCYAHGWDSKPVTAVVTMFSMDFLDLVIDGIEIGRMIIHELTTPKAQGLSEINQVLSIAKNAKDVIKMLIEIFCKVIERITGEHGKKIQWAQDKKEEIMNVLERAEKWITTSDDHSEGIECLKLVRSIQSVIRGEESLKELAGELRAVGTHVLNKLGRLDKPNAPILVRAEPTVLYLYGNRGGGKSLASMAIAVKLCKELGISHVEGIYTKPIMSDFWDGYAGQPVVIMDDLGQSTSDEDWTNFCQLVSSCPLRLNMANLEKKGTQFNSPFIIASSNLSHPCPKTVYCTDAIARRLHIKVKVSPKEEFSTHAMLDVAKAKKAGAYCNLDCLDFQKISDLASTPVSVQDIVLALLHTNVDKQTVMGNIIQYWAQSNPREVFDTMAEGKNSGKYLWLFEKIKTSKWYILGCVGAALSVSVLGVFAYHMIKNHFRDQQHDQSAYSAAIKPLRVVRLEQSDAQSVVDISNVVHGNLVRVGVGPNEARIHWLNNGWGVYNTYILMPYHGIKDADVDDDLYIERAGTIYSTNMKMVQVLFLESREGDLVLINVPRLPKFRDIRNHFSTEENIRRAEGMPGTLCTLDHERFTLVTESDLKMVEAATYVCEDDKGVRTDISVGRSWKAKACTVAGMCGGALVTSNNKMQNAIVGIHVAGGAPAISRVITKEMIEEMLKTRAQCSRIWKTEFVEKKISVGSKTKYHKSPLYDFCPQKVIKCPTKLFYQGEIDVMQVMLAKYSSPIVSEPLGYATVVEAYTNRMVSFFSEPRQLTYDECINGIEGLDAIDLKTSAGFPYNTLSLKKSDLIINGKKAQRLQQDVEKMEEDLHMNRSIQVVFTTCAKDELRPLSKGMLGKTRAIKACPVSFTILFRKYLGYALAQIQSHPGFHTGIAVGVDPDQDWHCMWYSIVTQCDLVVGLDFSNYDASLSPFMIYHAGRVLGQICGLDPRLVDRIMEPIVNSVHQLGSMRYYVDGSMPSGTPATSVLNSIINVVNISHVLCALEKISVFEVFKLSKILTYGDDVLFCIKKESLDQKSFPLSSFVQGLKELGMSPTGADKMEVKVTPVHKMSFLKRTFYVDEWSICHPRISEETVYSMLAWKSDNASMKHVIETSIWFMFHHGPRKYVIFCTCLRGVLCRVGIGLYIPTYKELEVRYDRLVKDRVIDDSF

SEQ ID NO: 149 - protein, 2124 aa, synthetic construct
MGANNSKESVSSNGNEGTIVNNFYSNQYYASIDASAQGVGTSTTPENGNVSGFLGLAGSAFNALSLLASPRTETGMMMEDRVLSRTAGNTSVNSQAAEGVLQAYGTETDSNSPTSCGDDPSKGTHATDRAFVIQLLPWKQTTNSYFAQWVRLTQKLSNNLHGNVMAKNIKSHAFAKMGFEVMLQANTSPFHNGILGLFLVPEFVRKGEITDEWIDLTPTSSLVSNTELYNPQTYANFPFDAKHSFDYSDITPEQFMIFPHQLINPKDTNVATVRVPYINIAPTNDTTVHTVWTAVVMVLVPLNFSSGASPTVSLTLTITPINSVFNGLHHTAQGPIPVRPFHNFQQFSTTVPLRTEPCYGMTVTPPVDYMPLPITDLVELAKVPSFVTVANSDTTSERSFPYFSVSNTEQGRNLFKSSVVLSDLHYQHTLVANLARYFCNYRGSLQFDFIAATTAMTRGKLLISYTPPGAGEPQSIDQAMMGTYAIWDLGLQSTFNFVVPFISASDFRFNTSSVSNALNSDGWITVWLMNPLTYPPSTPPTQQILMLMSAGSDFSYRLPISPGFAEGETSEHPMDNAECGKIDDKDAGMFSGHSVGLPTPHTSTSFFYDRYRFVGIVKSVVNNTPKPVNIYDDTGKVKNLQQVFPTSDTLLPHSLMSLSPCASVCGQPISSFLFAQRANPKKTLKLRSGDEFLYRCCPFSYIKCDLEFTVVPPANSTRDYIVHWYPPGATLDAGEVAVGNTSGSNGFDDNGMNAGSSLFSYNPTFHARAPSKVSAVIPFCLPVSLLPLYFDGFPDYSTTKGMYGCSPSFSFGTIYIESGLQETYSVYIRYKDFKGYAPRPLIRTPHIRLSERARYIMADSVLPRPLTRAERDVARDLLLIAGDIESNPGPAFNPEYTAHGPVTELIQLARKPETVDNVNRLLTTLNTLMAKWNNLKDTVTDAVFLRDMVCLLVKLTSLMYLVHGQGPGAYFAAASILLADGITFFDWYEKIKIFMARKLRVSPPFFPAAQGPDLRDFVTFFNAARGAQWMIDSLKSLITCIKQWLELEEENEAVQLEKMLIDSPRHCKAINDYNRGDSFQRPTNSFEFMDRLVECATKLGKVQIATYFRNFTTADSDTSRPEPVVVVLRGKPGVGKSAAATVMAAAVSKLLVGSQSVYTLSPDTEHMDGYHGQFVTLMDDLGQNPDGEDFRCFCQMVSCAQYRPAMADLKDKGILFTSRLLIATTNLPDFNPVTISDPRALDRRITFDILVTPGSAATKNGKLDLAAALKPDGPGEHPYTSDCPILHTTGLLLKNLRNNQTMNLKDLVDMIVKRIKHKKEVGNMLDSLVAQGPTMIVGYTKDDDGIAIVDCLEEWNKIKDKKKKQLALEMVAQELKDKHEEHKGTIKLLKMFVTGLGVVAAVAGAYATMKYFTKDKPKEEEEEPEEKKEKKTEESKEAAGPYNGPTKKEIKTLKLKAQSPLMDMEKKIAQNVMPFQIFYNGKRYTQSCLAIGKRVILVNKHAFESVEHKFVVDQKEYTLDQVTAISLDCGSGVTDVCAVCLPPGPDFKSIKKHFLPFNTTMFPGTRLTILSNDHYPMSREGSFLRFEDEVPTNVGNMPFVMLYKSTSYFGMCGSVVCSRFVDGGGIIGMHCAGGGGVSVGTRLTARMIESVFDYFYPPVAQGIIENTETGPRVHVPRTSKLKRTNATYPATEKYGPAALSRYDPRLNEGVNLDEVIFSKHTQNTLVEKGSTFRSALDMAAEIYGEKFRGNDFSPLSVEDAILGIPGLDRLDPNTASGLPYTKTRRQMIDFNTGQILDDTLKCRLGQWLAGRPPQEVHYQTFLKDEIRPIEKVKAGKTRIIDVPPLDHVIAFRMLFGRFIAHYHLNFGFKTGSAIGCDPDVAWASFGFELSGFPYLYDFDYSNFDASHSTSIFEILEQKFFSPELGFDPRCSLLLKSLAVSTHCYENKRLQIAGGLPSGTAGTSVLNTVINNIIFHGALYHTYTNFERDDISMLAYGDDIVVASKFELDLVMVKAFMNRIGYKITPADKSDEFRPKCMDDICFLKRRFVKVAGVWAPVMETENLEAMLSWYKPGTLNEKLQSVSRLAHFSGRDVYDHLFKPFIRDGFDVTPWKQLHLEWLNKLSA

SEQ ID NO: 150 - protein, 2181 aa, synthetic construct
MQNSHFSFDTASGTFEDVTGTKVKIVEYPRSVNNGVYDSSTHLEILNLQGEIEILRSFNEYQIRAAKQQLGLDIVYELQGNVQTTSKNDFDSRGNNGNMTFNYYANTYQNSVDFSTSSSASGAGPGNSRGGLAGLLTNFSGILNPLGYLKDHNTEEMENSADRVTTQTAGNTAINTQSSLGVLCAYVEDPTKSDPPSSSTDQPTTTFTAIDRWYTGRLNSWTKAVKTFSFQAVPLPGAFLSRQGGLNGGAFTATLHRHFLMKCGWQVQVQCNLTQFHQGALLVAMVPETTLDVKPDGKAKSLQELNEEQWVEMSDDYRTGKNMPFQSLGTYYRPPNWTWGPNFINPYQVTVFPHQILNARTSTSVDINVPYIGETPTQSSETQNSWTLLVMVLVPLDYKEGATTDPEITFSVRPTSPYFNGLRNRYTAGTDEEQGPIPTAPRENSLMFLSTLPDDTVPAYGNVRTPPVNYLPGEITDLLQLARIPTLMAFERVPEPVPASDTYVPYVAVPTQFDDRPLISFPITLSDPVYQNTLVGAISSNFANYRGCIQITLTFCGPMMARGKFLLSYSPPNGTQPQTLSEAMQCTYSIWDIGLNSSWTFVVPYISPSDYRETRAITNSVYSADGWFSLHKLTKITLPPDCPQSPCILFFASAGEDYTLRLPVDCNPSYVFHSTDNAETGVIEAGNTDTDFSGELAAPGSNHTNVKFLFDRSRLLNVIKVLEKDAVFPRPFPTQEGAQQDDGYFCLLTPRPTVASRPATRFGLYANPSGSGVLANTSLDFNFYSLACFTYFRSDLEVTVVSLEPDLEFAVGWFPSGSEYQASSFVYDQLHVPFHFTGRTPRAFASKGGKVSFVLPWNSVSSVLPVRWGGASKLSSATRGLPAHADWGTIYAFVPRPNEKKSTAVKHVAVYIRYKNARAWCPSMLPFRSYKQKMLMQSGDIETNPGPASDNPILEFLEAENDLVTLASLWKMVHSVQQTWRKYVKNDDFWPNLLSELVGEGSVALAATLSNQASVKALLGLHFLSRGLNYTDFYSLLIEKCSSFFTVEPPPPPAENLMTKPSVKSKFRKLFKMQGPMDKVKDWNQIAAGLKNFQFVRDLVKEVVDWLQAWINKEKASPVLQYQLEMKKLGPVALAHDAFMAGSGPPLSDDQIEYLQNLKSLALTLGKTNLAQSLTTMINAKQSSAQRVEPVVVVLRGKPGCGKSLASTLIAQAVSKRLYGSQSVYSLPPDPDFFDGYKGQFVTLMDDLGQNPDGQDFSTFCQMVSTAQFLPNMADLAEKGRPFTSNLIIATTNLPHFSPVTIADPSAVSRRINYDLTLEVSEAYKKHTRLNFDLAFRRTDAPPIYPFAAHVPFVDVAVRFKNGHQNFNLLELVDSICTDIRAKQQGARNMQTLVLQSPNENDDTPVDEALGRVLSPAAVDEALVDLTPEADPVGRLAILAKLGLALAAVTPGLIILAVGLYRYFSGSDADQEETESEGSVKAPRSENAYDGPKKNSKPPGALSLMEMQQPNVDMGFEAAVAKKVVVPITFMVPNRPSGLTQSALLVTGRTFLINEHTWSNPSWTSFTIRGEVHTRDEPFQTVHFTHHGIPTDLMMVRLGPGNSFPNNLDKFGLDQMPARNSRVVGVSSSYGNFFFSGNFLGFVDSITSEQGTYARLFRYRVTTYKGWCGSALVCEAGGVRRIIGLHSAGAAGIGAGTYISKLGLIKALKHLGEPLATMQGLMTELEPGITVHVPRKSKLRKTTAHAVYKPEFEPAVLSKFDPRLNKDVDLDEVIWSKHTANVPYQPPLFYTYMSEYAHRVFSFLGKDNDILTVKEAILGIPGLDPMDPHTAPGLPYAINGLRRTDLVDFVNGTVDAALAVQIQKFLDGDYSDHVFQTFLKDEIRPSEKVRAGKTRIVDVPSLAHCIVGRMLLGRFAAKFQSHPGFLLGSAIGSDPDVFWTVIGAQLEGRKNTYDVDYSAFDSSHGTGSFEALISHFFTVDNGFSPALGPYLRSLAVSVHAYGERRIKITGGLPSGCAATSLLNTVLNNVIIRTALALTYKEFEYDMVDIIAYGDDLLVGTDYDLDFNEVARRAAKLGYKMTPANKGSVFPPTSSLSDAVFLKRKFVQNNDGLYKPVMDLKNLEAMLSYFKPGTLLEKLQSVSMLAQHSGKEEYDRLMHPFADYGAVPSHEYLQARWRALFD

SEQ ID NO: 151 - protein, 2290 aa, synthetic construct
MATTMEQETCAHSLTFEECPKCSALQYRNGFYLLKYDEEWYPEELLTDGEDDVFDPELDMEVVFELQGNSTSSDKNNSSSEGNEGVIINNFYSNQYQNSIDLSANAAGSDPPRLRSIFESLSGAVNAFSNMLPLLADQNTEEMENLSDRGLKTLPAIRSQTPSQQWAVLSVMVPFMMESIRHHVLTLLQKRFWRWKGTTPSRLMIGHQHKSPLSTSAFPFLTSCPVKMVVSLVALRRHYLVKTGWRVQVQCNASQFHAGGLLVFMAPEYPTLDAFAMDNRWSKDNLPNGTRTQTNKKGPFAMDHQNFWQWTLYPHQFLNLRTNTTVDLEVPYVNIAPTSSWTQHASWTLVIAVVAPLTYSTGASTSLDITASIQPVRPVFNGLRHETLSRQSPIPVTIREHAGTWYSTLPDSTVPIYGKTPVAPSNYMVGEYKDFLEIAQIPTFIGNKIPNAVPYIEASNTAVKTQPLATYQVTLSCSCLANTFLAALSRNFAQYRGSLVYTFVFTGTAMMKGKFLIAYTPPGAGKPTSRDQAMQATYAIWDLGLNSSYSFTVPFISPTHFRMVGTDQVNITNADGWVTVWQLTPLTYPPGCPTSAKILTMVSAGKDFSLKMPISPAPWSPQGVENAEKGVTENTNATADFVAQPVYLPENQTKVAFFYNRSSPIGAFTVKSGSLESGFAPFSNGTCPNSVILTPGPQFDPAYDQLRPQRLTEIWGNGNEETSKVFPLKSKQDYSFCLFSPFVYYKCDLEVTLSPHTSGNHGLLVRWCPTGTPTKPTTQVLHEVSSLSEGRTPQVYSAGPGISNQISFVVPYNSPLSVLSAVWYNGHKRFDNTGSLGIAPNSDFGTLFFAGTKPDIKFTVYLRYKNKRVFCPRPTVFFPWPTSGDKIDMTPRAGVLMLESPNALDISRTYPTLHVLIQFNHRGLEVRLFRHGHFWAETRADVILRSKTKQVSFLSNGNYPSMDSRAPWNPWKNTYQAVLRAEPCRVTMDIYYKRVRPFRLPLVQKEWPVREENVFGLYRIFNAHYAGYFADLLIHDIETNPGPFMFRPRKQVFQTQGAAVSSMAQTLLPNDLASKAMGSAFTALLDANEDAQKAMKIIKTLSSLSDAWENVKETLNNPEFWKQLLSRCVQLIAGMTIAVMHPDPLTLLCLGTLTAAEITSQTSLCEEIAAKFKTIFITPPPRFPTISLFQQQSPLKQVNDIFSLAKNLDWAVKTVEKVVDWFGTWIVQEEKEQTLDQLLQRFPEHAKRISDLRNGMAAYVECKESFDFFEKLYNQAVKEKRTGIAAVCEKFRQKHDHATARCEPVVIVLRGDAGQGKSLSSQVIAQAVSKTIFGRQSVYSLPPDSDFFDGYENQFAAIMDDLGQNPDGSDFTTFCQMVSTTNFLPNMASLERKGTPFTSQLVVATTNLPEFRPVTIAHYPAVERRITFDYSVSAGPVCSKTEAGYKVLDVERAFRPTGEAPLPCFQNNCLFLEKAGLQFRDNRTKEIISLVDVIERAVARIERKKKVLTTVQTLVAQGPVDEVSFHSVVQQLKARQQATDEQLEELQEAFAKVQERNSVFSDWLKISAMLCAATLALSQVVKMAKAVKQMVKPDLVRVQLDEQEQGPYNETARVKPKTLQLLDIQGPNPVMDFEKYVAKHVTAPIGFVYPTGVSTQTCLLVRGRTLVVNRHMAESDWTSIVVRGVTHARSTVKILAIAKAGKETDVSFIRLSSGPLFRDNTSKFVKAGDVLPTGAAPVTGIMNTDIPMMYTGTFLKAGVSVPVETGQTFNHCIHYKANTRKGWCGSALLADLGGSKKILGIHSAGSMGIAAASIVSQEMIRAVVNAFEPQGALERLPDGPRIHVPRKTALRPTVARQVFQPAYAPAVLSKFDPRTEADVDEVAFSKHTSNQESLPPVFRMVAKEYANRVFTLLGKDNGRLTVKQALEGLEGMDPMDRNTSPGLPYTALGMRRTDVVDWESATLIPFAAERLRKMNEGDFSEVVYQTFLKDELRPIEKVQAAKTRIVDVPPFEHCILGRQLLGKFASKFQTQPGLELGSAIGCDPDVHWTAFGVAMQGFERVYDVDYSNFDSTHSVAMFRLLAEEFFTPENGFDPLTREYLESLAISTHAFEEKRFLITGGLPSGCAATSMLNTIMNNIIIRAGLYLTYKNFEFDDVKVLSYGDDLLVATNYQLDFDKVRASLAKTGYKITPANTTSTFPLNSTLEDVVFLKRKFKKEGPLYRPVMNREALEAMLSYYRPGTLSEKLTSITMLAVHSGKQEYDRLFAPFREVGVVVPSFESVEYRWRSLFW

SEQ ID NO: 152 - protein, 2293 aa, synthetic construct
MATTMEQEICAHSMTFEECPKCSALQYRNGFYLLKYDEEWYPEELLTDGEDDVFDPDLDMEVVFETQGNSTSSDKNNSSSEGNEGVIINNFYSNQYQNSIDLSANATGSDPPKTYGQFSNLLSGAVNAFSNMLPLLADQNTEEMENLSDRVSQDTAGNTVTNTQSTVGRLVGYGTVHDGEHPASCADTASEKILAVERYYTFKVNDWTSTQKPFEYIRIPLPHVLSGEDGGVFGATLRRHYLVKTGWRVQVQCNASQFHAGSLLVFMAPEYPTLDVFAMDNRWSKDNLPNGTRTQTNRKGPFAMDHQNFWQWTLYPHQFLNLRTNTTVDLEVPYVNIAPTSSWTQHASWTLVIAVVAPLTYSTGASTSLDITASIQPVRPVFNGLRHEVLSRQSPIPVTIREHAGTWYSTLPDSTVPIYGKTPVAPANYMVGEYKDFLEIAQIPTFIGNKVPNAVPYIEASNTAVKTQPLAVYQVTLSCSCLANTFLAALSRNFAQYRGSLVYTFVFTGTAMMKGKFLIAYTPPGAGKPTSRDQAMQATYAIWDLGLNSSYSFTVPFISPTHFRMVGTDQANITNVDGWVTVWQLTPLTYPPGCPTSAKILTMVSAGKDFSLKMPISPAPWSPQGVENAEKGVTENTDATADFVAQPVYLPENQTKVAFFYDRSSPIGAFAVKSGSLESGFAPFSNKACPNSVILTPGPQFDPAYDQLRPQRLTEIWGNGNEETSEVFPLKTKQDYSFCLFSPFVYYKCDLEVTLSPHTSGAHGLLVRWCPTGTPTKPTTQVLHEVSSLSEGRTPQVYSAGPGTSNQISFVVPYNSPLSVLPAVWYNGHKRFDNTGDLGIAPNSDFGTLFFAGTKPDIKFTVYLRYKNMRVFCPRPTVFFPWPTSGDKIDMTPRAGVLMLESPNPLDVSKTYPTLHILLQFNHRGLEARIFRHGQLWAETHAEVVLRSKTKQISFLSNGSYPSMDATTPLNPWKSTYQAVLRAEPHRVTMDVYHKRIRPFRLPLVQKEWRTCEENVFGLYHVFETHYAGYFSDLLIHDVETNPGPFTFKPRQRPVFQTQGAAVSSMAQTLLPNDLASKAMGSAFTALLDANEDAQKAMKIIKTLSSLSDAWENVKGTLNNPEFWKQLLSRCVQLIAGMTIAVMHPDPLTLLCLGVLTAAEITSQTSLCEEIAAKFKTIFTTPPPRFPVISLFQQQSPLKQVNDVFSLAKNLDWAVKTVEKVVDWFGTWVAQEEREQTLDQLLQRFPEHAKRISDLRNGMAAYVECKESFDFFEKLYNQAVKEKRTGIAAVCEKFRQKHDHATARCEPVVIVLRGDAGQGKSLSSQIIAQAVSKTIFGRQSVYSLPPDSDFFDGYENQFAAIMDDLGQNPDGSDFTTFCQMVSTTNLLPNMASLERKGTPFTSQLVVATTNLPEFRPVTIAHYPAVERRITFDYSVSAGPVCSKTEAGCKVLDVERAFRPTGDAPLPCFQNNCLFLEKAGLQFRDNRSKEILSLVDVIERAVTRIERKKKVLTAVQTLVAQGPVDEVSFYSVVQQLKARQEATDEQLEELQEAFARVQERSSVFSDWMKISAMLCAATLALTQVVKMAKAVKQMVRPDLVRVQLDEQEQGPYNETTRIKPKTLQLLDVQGPNPTMDFEKFVAKFVTAPIGFVYPTGVSTQTCLLVKGRTLAVNRHMAESDWTSIVVRGVSHTRSSVKIIAIAKAGKETDVSFIRLSSGPLFRDNTSKFVKASDVLPHSSSPLIGIMNVDIPMMYTGTFLKAGVSVPVETGQTFNHCIHYKANTRKGWCGSAILADLGGSKKILGFHSAGSMGVAAASIISQEMIDAVVQAFEPQGALERLPDGPRIHVPRKTALRPTVARQVFQPAFAPAVLSKFDPRTDADVDEVAFSKHTSNQETLPPVFRMVAREYANRVFALLGRDNGRLSVKQALDGLEGMDPMDKNTSPGLPYTTLGMRRTDVVDWETATLIPFAAERLEKMNNKDFSDIVYQTFLKDELRPIEKVQAAKTRIVDVPPFEHCILGRQLLGKFASKFQTQPGLELGSAIGCDPDVHWTAFGVAMQGFERVYDVDYSNFDSTHSVAVFRLLAEEFFSEENGFDPLVKDYLESLAISKHAYEEKRYLITGGLPSGCAATSMLNTIMNNIIIRAGLYLTYKNFEFDDVKVLSYGDDLLVATNYQLNFDRVRTSLAKTGYKITPANKTSTFPLESTLEDVVFLKRKFKKEGPLYRPVMNREALEAMLSYYRPGTLSEKLTSITMLAVHSGKQEYDRLFAPFREVGVIVPTFESVEYRWRSLFW

SEQ ID NO: 153 - protein, 2295 aa, synthetic construct
MACKHGYPLLCPLCTALDITPDGSFTLLFDNEWYPTDLLTVNLDDDVFYPLDTNMDWTDLPLIQDIVMEPQGNSNSSDKNNSQSSGNEGVIINNYYSNQYQNSIDLSANANGVGKENSKPQGQLMNILGSAADAFKNIAPLLMDQNTEEMTNLSDRVSSDTAGNTATNTQSTVGRLFGFGQRHKGKHPASCADTATDKVLAAERYYTIKLASWTKTQESFDHIRVPLPHALAGENGGVFSSTLRRHYLCKCGWRIQVQCNASQFHAGSLLVFMAPEFDTSNHSTEVEPRADTAFKVDANWQKHAQILTGHAYVNTTTKVNVPLALNHQNFWQWTTYPHQILNLRTNTTCDLEVPYVNVCPTSSWTQHANWTLVIAVLTPLQYSQGSATTIEITASIQPVKPVFNGLRHTVVNPQSPFPVTVREHAGTFFSTTPDTTVPVYGNTISTPFDYMCGEFTDLLSLCKIPTFLGNLDSNKKRIPYFSATNSTPATPLVTYQVTLSCSCMANSMLAAVARNFNQYRGSLNYLFVFTGSAMTKGKFLISYTPPGAGEPKTLDQAMQATYAIWDLGLNSSYNFTVPFISPTHYRQTSYNTPTITSVDGWLTVWQLTPLTYPLGVPNDSHILTLVSGGDDFTLRMPVTFTKYVPQGVDNAEKGKVSDDNASTDFVAEPVKLPENQTRVSFVYDRSTLSSVLQSTSDVSSKFTPSTAKNLQNSILLTPLPSDIVNNSVLPEQERWISFASPTTQKPPYKTKQDWNFIMFSPFTYYKCDLEVTLSKNDRETISSVVRYVPCGAPSDLSDQTMPQTPSLADTRDPHMWVVGQGTTNQISFVIPYTSPLSVLPSVWFNGFSNFDNSSRFGVAPNADFGRLLLQGQGTFSVHYRYKKMRVFCPRPTVFIPWPNPQDTKIKSVRPTPTLELQNPISIYRVDLFINFSDEIIQFTYKVHGRTVCQYEIPGFGLSRSGRLLVCMGEKPCQLPISTPKCFYHIVFTGSRNSFGVSIYKARYRPWKQPLHDELHDYGFSTFTDFFKAVRDYHASYYKQRLQHDIETNPGPVQSVFQLQGGVLTKSQAPMSGLQSMLLRAIGIEADCTEFTRAVNLITDLCNTWESAKTTLSSPEFWTKMVMRIVKMFAASVLYLHNPDLTTTVCLSLMAGIDILTNDSVFNWLSTKLSKFFHTPAPPIVPLLQQQSPIREANDSFNLAKNIEWAIKTIKRIVEWITSWFKQEETSPQAKLDKMLTDFPEHCNSILAMRNGRKAYTDCASAFKYFEQLYNLAVQCKRIPLATLCEKFKNKHDHAVARPEPVVVVLRGNAGQGKSVTSQIIAQAVSKLSFGRQSVYSLPPDSDYLDGYENQYSVIMDDLGQNPDGEDFKVFCQMVSSTNFLPNMAHLEKKGTPFTSNFIIATTNLPKFRPVTVAHYPAVDRRITFDLTVEAGDECVTHNGMLDVEKAFEEIPGKPQLDCFNTDCRLLHKRGVRFVCNRTKNIYNLQQVVKMVKSTIDNKVENLKKMNTLVAQSPGNDMDYVLTCLRQTNAALQDQIDELQEAFNQAQERQNFLSDWLKVSAIVFASIASLSAVCKLVSRFKNLVCPAPVQIQLSEGEQAAYSGGKKGEKQTLQVLDVQGGGKIVAQAGNPVMDYEVNIAKNMVNPITFFYADKAQVTQSCLLIKGHLFVVNRHVAETDWCAFELKGTRHERDSVQMRSVNKSGMEVDLTFVKVVKGPLFKDNSKKFCSKDDDFPARNETVTGIMNTGVPFVFTGKFLIGNQPVNTTTGACFNHCIHYRATTHRGWCGSALICHVNGKKAVYAMHSAGGGGMAAATIITQEMIEAAEKALDCLTPQGAIVEIGIDTVVHVPRKTKLRRTVAHPCFQPKFEPAVLSRYDPRTTKDVDQVAFSKHTTNLEELPSVFSMVAREYATRVFTTIGKENKILTPEQAILGLPGMDPMEKDTSPGLPYTQQGLKRAQLVNFEQGTMVQNLKEAHTKLTEGNYEDILYQSFLKDEIRPIEKIHEAKTRIVDVPPFHHCIWGRQLLGRFASRFQTNPGLDLGSAIGTDPDTDWTAFAFQLLQYKYVYDVDYSNFDASHSTAMFEVLIENFFTTENGFDERIGDYLRSLAVSRHAFEERRVLVRGGLPSGCAATSMLNTIINNIVIRAALHLTYSNFEFDDIKVLSYGDDLLIATNYQINFNLVKQRLAPFNYKITPANKTVEFPEISNLYEVTFLKRKFVRYNSCLFKPQMDTENLKAMVSYCRPGTLKEKLNSIALLAVHSGKSVYDEIFDPFRRIGIIIPEHGTMLYRWLNLFR

SEQ ID NO: 154 - protein, 2303 aa, synthetic construct
MACKHGYPDVCPICTAVDATPGFEYLLMADGEWYPTDLLCVDLDDDVFWPSDTSNQSQTMDWTDVPLIRDIVMEPQGNSSSSDKSNSQSSGNEGVIINNFYSNQYQNSIDLSASGGNAGDAPQTNGQLSNILGGAANAFATMAPLLLDQNTEEMENLSDRVASDKAGNSATNTQSTVGRLCGYGKSHHGEHPASCADTATDKVLAAERYYTIDLASWTTSQEAFSHIRIPLPHVLAGEDGGVFGATLRRHYLCKTGWRVQVQCNASQFHAGSLLVFMAPEFYTGKGTKTGTMEPSDPFTMDTEWRSPQGAPTGYRYDSRTGFFATNHQNQWQWTVYPHQILNLRTNTTVDLEVPYVNVAPSSSWTQHANWTLVVAVLSPLQYATGSSPDVQITASLQPVNPVFNGLRHETVIAQSPIPVTVREHKGCFYSTNPDTTVPIYGKTISTPSDYMCGEFSDLLELCKLPTFLGNPNTNNKRYPYFSATNSVPATSMVDYQVALSCSCMANSMLAAVARNFNQYRGSLNFLFVFTGAAMVKGKFLIAYTPPGAGKPTTRDQAMQSTYAIWDLGLNSSFNFTAPFISPTHYRQTSYTSPTITSVDGWVTVWKLTPLTYPSGTPTNSDILTLVSAGDDFTLRMPISPTKWVPQGVDNAEKGKVSNDDASVDFVAEPVKLPENQTRVAFFYDRAVPIGMLRPGQNMETTFNYQENDYRLNCLLLTPLPSFCPDSSSGPQKTKAPVQWRWVRSGGVNGANFPLMTKQDYAFLCFSPFTFYKCDLEVTVSALGMTRVASVLRWAPTGAPADVTDQLIGYTPSLGETRNPHMWLVGAGNSQVSFVVPYNSPLSVLPAAWFNGWSDFGNTKDFGVAPNADFGRLWIQGNTSASVRIRYKKMKVFCPRPTLFFPWPTPTTTKINADNPVPILELENPAALYRIDLFITFTDEFITFDYKVHGRPVLTFRIPGFGLTPAGRMLVCMGEQPAHGPFTSSRSLYHVIFTATCSSFSFSIYKGRYRSWKKPIHDELVDRGYTTFGEFFKAVRGYHADYYRQRLIHDVETNPGPVQSVFQPQGAVLTKSLAPQAGIQNLLLRLLGIDGDCSEVSKAITVVTDLVAAWEKAKTTLVSPEFWSKLILKTTKFIAASVLYLHNPDFTTTVCLSLMTGVDLLTNDSVFDWLKQKLSSFFRTPPPACPNVMQPQGPLREANEGFTFAKNIEWAMKTIQSVVNWLTSWFKQEEDHPQSKLDKLLMEFPDHCRNIMDMRNGRKAYCECTASFKYFDELYNLAVTCKRIPLASLCEKFKNRHDHSVTRPEPVVVVLRGAAGQGKSVTSQIIAQSVSKMAFGRQSVYSMPPDSEYFDGYENQFSVIMDDLGQNPDGEDFTVFCQMVSSTNFLPNMAHLERKGTPFTSSFIVATTNLPKFRPVTVAHYPAVDRRITFDFTVTAGPHCKTPAGMLDVEKAFDEIPGSKPQLACFSADCPLLHKRGVMFTCNRTQTVYNLQQVVKMVNDTITRKTENVKKMNSLVAQSPPDWEHFENILTCLRQNNAALQDQLDELQEAFAQARERSDFLSDWLKVSAIIFAGIASLSAVIKLASKFKESIWPTPVRVELSEGEQAAYAGRARAQKQALQVLDIQGGGKVLAQAGNPVMDFELFCAKNIVAPITFYYPDKAEVTQSCLLLRAHLFVVNRHVAETDWTAFKLKDVRHERHTVALRSVNRSGAKTDLTFIKVTKGPLFKDNVNKFCSNKDDFPARNDTVTGIMNTGLAFVYSGNFLIGNQPVNTTTGACFNHCLHYRAQTRRGWCGSAIICNVNGKKAVYGMHSAGGGGLAAATIITKELIEAAEKSMLALEPQGAIVDIATGSVVHVPRKTKLRRTVAHDVFQPKFEPAVLSRYDPRTDKDVDVVAFSKHTTNMESLPPIFDVVCGEYANRVFTILGKENGLLTVEQAVLGLPGMDPMEKDTSPGLPYTQQGLRRTDLLNFITAKMTPQLDYAHSKLVIGVYDDVVYQSFLKDEIRPIEKIHEAKTRIVDVPPFAHCIWGRQLLGRFASKFQTKPGLELGSAIGTDPDVDWTRYAVELSGFNYVYDVDYSNFDASHSTAMFECLINNFFTEQNGFDRRIAEYLRSLAVSRHAYEDRRVLIRGGLPSGCAATSMLNTIMNNVIIRAALYLTYSNFDFDDIKVLSYGDDLLIGTNYQIDFNLVKERLAPFGYKITPANKTTTFPLTSHLQDVTFLKRRFVRFNSYLFRPQMDAVNLKAMVSYCKPGTLKEKLMSIALLAVHSGPDIYDEIFLPFRNVGIVVPTYSSMLYRWLSLFR

SEQ ID NO: 155 - protein, 2303 aa, synthetic construct
MACKHGYPDVCPICTAVDATPDFEYLLMADGEWFPTDLLCVDLDDDVFWPSDTSTQPQTMEWTDVPLVCDTVMEPQGNASSSDKSNSQSSGNEGVIINNFYSNQYQNSIDLSASGGNAGDAPQNNGQLSSILGGAANAFATMAPLLMDQNTEEMENLSDRVASDKAGNSATNTQSTVGRLCGYGKSHHGEHPTSCADAATDKVLAAERYYTIDLASWTTSQEAFSHIRIPLPHVLAGEDGGVFGATLRRHYLCKTGWRVQVQCNASQFHAGSLLVFMAPEFYTGKGTKSGTMEPSDPFTMDTTWRSPQSAPTGYRYDRQAGFFAMNHQNQWQWTVYPHQILNLRTNTTVDLEVPYVNVAPSSSWTQHANWTLVVAVLSPLQYATGSSPDVQITASLQPVNPVFNGLRHETVLAQSPIPVTVREHQGCFYSTNPDTTVPIYGKTISTPSDYMCGEFSDLLELCKLPTFLGNPSTDNKRYPYFSATNSVPATSLVDYQVALSCSCTANSMLAAVARNFNQYRGSLNFLFVFTGAAMVKGKFRIAYTPPGAGKPTTRDQAMQATYAIWDLGLNSSFNFTAPFISPTHYRQTSYTSPTITSVDGWVTVWQLTPLTYPSGTPTHSDILTLVSAGDDFTLRMPISPTKWVPQGIDNAEKGKVSNDDASVDFVAEPVKLPENQTRVAFFYDRAVPIGMLRPGQNMETTFSYQENDFRLNCLLLTPLPSYCPDSSSGPVRTKAPVQWRWVRSGGANGANFPLMTKQDYAFLCFSPFTYYKCDLEVTVSAMGAGTVSSVLRWAPTGAPADVTDQLIGYTPSLGETRNPHMWIVGSGNSQISFVVPYNSPLSVLPAAWFNGWSDFGNTKDFGVAPTSDFGRIWIQGNSSASVRIRYKKMKVFCPRPTLFFPWPTPTTTKINADNPVPILELENPASLYRIDLFITFTDELITFDYKVHGRPVLTFRIPGFGLTPAGRMLVCMGAKPAHSPFTSSKSLYHVIFTSTCNSFSFTIYKGRYRSWKKPIHDELVDRGYTTFREFFKAVRGYHADYYKQRLIHDVEMNPGPVQSVFQPQGAVLTKSLAPQAGIQNILLRLLGIEGDCSEVSKAITVVTDLVAAWEKAKTTLVSPEFWSELILKTTKFIAASVLYLHNPDFTTTVCLSLMTGVDLLTNDSVFDWLKSKLSSFFRTPPPACPNVMQPQGPLREANEGFTFAKNIEWATKTIQSIVNWLTSWFKQEEDHPQSKLDKLLMEFPDHCRNIMDMRNGRKAYCECTASFKYFDDLYNLAVTCKRIPLASLCEKFKNRHDHSVTRPEPVVAVLRGAAGQGKSVTSQIIAQSVSKMAFGRQSVYSMPPDSEYFDGYENQFSVIMDDLGQNPDGEDFTVFCQMVSSTNFLPNMAHLERKGTPFTSSFIVATTNLPKFRPVTVAHYPAVDRRITFDFTVTAGPHCKTPAGMLDIEKAFDEIPGSKPQLACFSADCPLLHKRGVMFTCNRTKTVYNLQQVVKMVNDTITRKTENVKKMNSLVAQSPPDWQHFENILTCLRQNNAALQDQVDELQEAFTQARERSDFLSDWLKVSAIIFAGIVSLSAVIKLASKFKESIWPTPVRVELSEGEQAAYAGRARAQKQALQVLDIQGGGKVLAQAGNPVMDFELFCAKNMVSPITFYYPDKAEVTQSCLLLRAHLFVVNRHVAETEWTAFKLRDVRHERDTVVMRSVNRSGAETDLTFVKVTKGPLFKDNVNKFCSNKDDFPARNDTVTGIMNTGLAFVYSGNFLIGNQPVNTTTGACFNHCLHYRAQTRRGWCGSAIICNVNGKKAVYGMHSAGGGGLAAATIITRELIEAAEKSMLALEPQGAIVDISTGSVVHVPRKTKLRRTVAHDVFQPKFEPAVLSRYDPRTDKDVDVVAFSKHTTNMESLPPIFDIVCGEYANRVFTILGKDNGLLTVEQAVLGLSGMDPMEKDTSPGLPYTQQGLRRTDLLDFNTAKMTPQLDYAHSKLVLGVYDDVVYQSFLKDEIRPLEKIHEAKTRIVDVPPFAHCIWGRQLLGRFASKFQTKPGFELGSAIGTDPDVDWTRYAAELSGFNYVYDVDYSNFDASHSTAMFECLINNFFTEQNGFDRRIAEYLRSLAVSRHAYEDRRVLIRGGLPSGCAATSMLNTIMNNVIIRAALYLTYSNFEFDDIKVLSYGDDLLIGTNYQIDFNLVKERLAPFGYKITPANKTTTFPLTSHLQDVTFLKRRFVRFNSYLFRPQMDAVNLKAMVSYCKPGTLKEKLMSIALLAVHSGPDIYDEIFLPFRNVGIVVPTYDSMLYRWLSLFR

SEQ ID NO: 156 - protein, 2327 aa, synthetic construct
MNTTDCFIAVVNAIKEVRALFLPRTAGKMEFTLHDGEKKVFYSRPNNHDNCWLNTILQLFRYVDEPFFDWVYNSPENLTLEAIKQLEELTGLELREGGPPALVIWNIKHLLHTGIGTASRPSEVCMVDGTDMCLADFHAGIFMKGREHAVFACVTSNGWYAIDDEDFYPWTPDPSDVLVFVPYDQEPLNEGWKASVQRKLKGAGQSSPATGSQNQSGNTGSIINNYYMQQYQNSMDTQLGDNAISGGSNEGSTDTTSTHTTNTQNNDWFSKLASSAFSGLFGALLADKKTEETTLLEDRILTTRNGHTTSTTQSSVGVTFGYATAEDSTSGPNTSGLETRVHQAERFFKMALFDWVPSQNFGHMHKVVLPHEPKGVYGGLVKSYAYMRNGWDVEVTAVGNQFNGGCLLVALVPEMGDISDREKYQLTLYPHQFINPRTNMTAHITVPYVGVNRYDQYKQHRPWTLVVMVVAPLTTNTAGAQQIKVYANIAPTNVHVAGELPSKEGIFPVACSDGYGNMVTTDPKTADPAYGKVYNPPRTALPGRFTNYLDVAEACPTFLMFENVPYVSTRTDGQRLLAKFDVSLAAKHMSNTYLAGLAQYYTQYTGTINLHFMFTGPTDAKARYMVAYVPPGMDAPDNPEEAAHCIHAEWDTGLNSKFTFSIPYISAADYAYTASHEAETTCVQGWVCVYQITHGKADADALVVSASAGKDFELRLPVDARQQTTTTGESADPVTTTVENYGGETQVQRRHHTDVAFVLDRFVKVTVSDNQHTLDVMQAHKDNIVGALLRAATYYFSDLEIAVTHTGKLTWVPNGAPVSALNNTTNPTAYHKGPVTRLALPYTAPHRVLATAYTGTTTYTASARGDLAHLTTTHARHLPTSFNFGAVKAETITELLVRMKRAELYCPRPILPIQPTGDRHKQPLVAPAKQLLNFDLLKLAGDVESNPGPFFFSDVRSNFSKLVETINQMQEDMSTKHGPDFNRLVSAFEELASGVKAIRTGLDEAKPWYKLIKLLSRLSCMAAVAARSKDPVLVAIMLADTGLEILDSTFVVKKISDSLSSLFHVPAPAFSFGAPILLAGLVKVASSFFRSTPEDLERAEKQLKARDINDIFAILKNGEWLVKLILAIRDWIKAWIASEEKFVTMTDLVPGILEKQRDLNDPSKYKDAKEWLDNTRQVCLKSGNVHIANLCKVVAPAPSKSRPEPVVVCLRGKSGQGKSFLANVLAQAISTHLTGRTDSVWYCPPDPDHFDGYNQQTVVVMDDLGQNPDGKDFKYFAQMVSTTGFIPPMASLEDKGKPFSSKVIIATTNLYSGFTPKTMVCPDALNRRFHFDIDVSAKDGYKINNKLDIIKALEDTHTNPVAMFQYDCALLNGMAVEMKRLQQDMFKPQPPLQNVYQLVQEVIERVELHEKVSSHPIFKQISIPSQKSVLYFLIEKGQHEAAIEFFEGMVHDSIKEELRPLIQQTSFVKRAFKRLKENFEIVALCLTLLANIVIMIRETHKRQKMVDDAVNEYIEKANITTDDQTLDEAEKNPLETSGASTVGFRERTLPGQKARDDVNSEPAQPTEEQPQAEGPYAGPLERQRPLKVRAKLPRQEGPYAGPMERQKPLKVKARAPVVKEGPYEGPVKKPVALKVKAKNLIVTESGAPPTDLQKMVMGNTKPVELILDGKTVAICCATGVFGTAYLVPRHLFAEKYDKIMLDGRALTDSDYRVFEFEIKVKGQDMLSDAALMVLHRGNRVRDITKHFRDVARMKKGTPVVGVINNADVGRLIFSGEALTYKDIVVCMDGDTMPGLFAYKAATKAGYCGGAVLAKDGADTFIVGTHSAGGNGVGYCSCVSRSMLLKMKAHIDPEPHHEGLIVDTRDVEERVHVMRKTKLAPTVAHGVFNPEFGPAALSNKDPRLNEGVVLDEVIFSKHKGDTKMSAEDKALFRRCAADYASRLHSVLGTANAPLSIYEAIKGVDGLDAMEPDTAPGLPWALQGKRRGALIDFENGTVGPEVEAALKLMEKREYKFACQTFLKDEIRPMEKVRAGKTRIVDVLPVEHILYTRMMIGRFCAQMHSNNGPQIGSAVGCNPDVDWQRFGTHFAQYRNVWDVDYSAFDANHCSDAMNIMFEEVFRTEFGFHPNAEWILKTLVNTEHAYENKRITVEGGMPSGCSATSIINTILNNIYVLYALRRHYEGVELDTYTMISYGDDIVVASDYDLDFEALKPHFKSLGQTITPADKSDKGFVLGHSITDVTFLKRHFHMDYGTGFYKPVMASKTLEAILSFARRGTIQEKLISVAGLAVHSGPDEYRRLFEPFQGLFEIPSYRSLYLRWVNAVCGDA

SEQ ID NO: 157 - protein, 2327 aa, synthetic construct
MNTTDCFIAVVNAIREIRALFLPRTTGKMEFTLHDGEKKVFYSRPNNHDNCWLNTILQLFRYVDEPFFDWVYNSPENLTLEAIKQLEELTGLELREGGPPALVIWNIKHLLHTGIGTASRPSEVCMVDGTDMCLADFHAGIFMKGQEHAVFACVTSNGWYAIDDEDFYPWTPDPSDVLVFVPYDQEPLNEGWKANVQRKLKGAGQSSPATGSQNQSGNTGSIINNYYMQQYQNSMDTQLGDNAISGGSNEGSTDTTSTHTTNTQNNDWFSKLASSAFSGLFGALLADKKTEETTLLEDRILTTRNGHTTSTTQSSVGVTFGYATAEDSTSGPNTSGLETRVHQAERFFKMALFDWVPSQNFGHMHKVVLPHEPKGVYGGLVKSYAYMRNGWDVEVTAVGNQFNGGCLLVALVPEMGDISDREKYQLTLYPHQFINPRTNMTAHITVPYVGVNRYDQYKQHRPWTLVVMVVAPLTTNTAGAQQIKVYANIAPTNVHVAGELPSKEGIFPVACSDGYGNMVTTDPKTADPAYGKVYNPPRTALPGRFTNYLDVAEACPTFLMFENVPYVSTRTDGQRLLAKFDVSLAAKHMSNTYLAGLAQYYTQYTGTINLHFMFTGPTDAKARYMVAYVPPGMDAPDNPEEAAHCIHAEWDTGLNSKFTFSIPYISAADYAYTASHEAETTCVQGWVCVYQITHGKADADALVVSASAGKDFELRLPVDARQQTTATGESADPVTTTVENYGGETQVQRRHHTDVAFVLDRFVKVTVSGNQHTLDVMQAHKDNIVGALLRAATYYFSDLEIAVTHTGKLTWVPNGAPVSALDNTTNPTAYHKGPLTRLALPYTAPHRVLATAYTGTTTYTASTRGDSAHLTATRARHLPTSFNFGAVKAETITELLVRMKRAELYCPRPILPIQPTGDRHKQPLVAPAKQLLNFDLLKLAGDVESNPGPFFFSDVRSNFSKLVETINQMQEDMSTKHGPDFNRLVSAFEELASGVKAIRTGLDEAKPWYKLIKLLSRLSCMAAVAARSKDPVLVAIMLADTGLEILDSTFVVKKISDSLSSLFHVPAPAFSFGAPILLAGLVKVASSFFRSTPEDLERAEKQLKARDINDIFAILKNGEWLVKLILAIRDWIKAWIASEEKFVTMTDLVPGILEKQRDLNDPSKYKDAKEWLDNTRQACLKSGNVHIANLCKVVAPAPSKSRPEPVVVCLRGKSGQGKSFLANVLAQAISTHLTGRTDSVWYCPPDPDHFDGYNQQTVVVMDDLGQNPDGKDFKYFAQMVSTTGFIPPMASLEDKGKPFSSKVIIATTNLYSGFTPKTMVCPDALNRRFHFDIDVSAKDGYKINNKLDIIKALEDTHTNPVAMFQYDCALLNGMAVEMKRLQQDMFKPQPPLQNVYQLVQEVIERVELHEKVSSHPIFKQISIPSQKSVLYFLIEKGQHEAAIEFFEGMVHDSIKEELRPLIQQTSFVKRAFKRLKENFEIVALCLTLLANIVIMIRETHKRQKMVDDAVNEYIEKANITTDDKTLDEAEKNPLETSGASTVGFRERTLPGQKARDDVNSEPAQPTEEQPQAEGPYAGPLERQRPLKVRAKLPQQEGPYAGPMERQKPLKVKARAPVVKEGPYEGPVKKPVALKVKAKNLIVTESGAPPTDLQKMVMGNTKPVELILDGKTVAICCATGVFGTAYLVPRHLFAEKYDKIMLDGRALTDSDYRVFEFEIKVKGQDMLSDAALMVLHRGNRVRDITKHFRDVARMKKGTPVVGVINNADVGRLIFSGEALTYKDIVVCMDGDTMPGLFAYKAATKAGYCGGAVLAKDGADTFIVGTHSAGGNGVGYCSCVSRSMLLKMKAHIDPEPHHEGLIVDTRDVEERVHVMRKTKLAPTVAHGVFNPEFGPAALSNKDPRLNEGVVLDEVIFSKHKGDTKMSEEDKALFRRCAADYASRLHSVLGTANAPLSIYEAIKGVDGLDAMEPDTAPGLPWALQGKRRGALIDFENGTVGPEVEAALKLMEKREYKFACQTFLKDEIRPMEKVRAGKTRIVDVLPVEHILYTRMMIGRFCAQMHSNNGPQIGSAVGCNPDVDWQRFGTHFAQYRNVWDVDYSAFDANHCSDAMNIMFEEVFRTEFGFHPNAEWILKTLVNTEHAYENKRITVEGGMPSGCSATSIINTILNNIYVLYALRRHYEGVELDTYTMISYGDDIVVASDYDLDFEALKPHFKSLGQTITPADKSDKGFVLGHSITDVTFLKRHFHMDYGTGFYKPVMASKTLEAILSFARRGTIQEKLISVAGLAVHSGPDEYRRLFEPFQGLFEIPSYRSLYLRWVNAVCGDA

SEQ ID NO: 158 - protein, 2336 aa, synthetic construct
MNTTDCFIALLYALREIKAFLLSRTQGKMELTLYNGEKKTFYSRPNNHDNCWLNTILQLFRYVDEPFFDWVYDSPENLTCEAIRQLEEITGLELHEGGPPALVIWNIKHLLHTGIGTASRPSEVCMVDGTDMCLADFHAGIFLKGQEHAVFACVTSDGWYAIDDEDFYPWTPDPSDVLVFVPYDQEPLNGEWKAKVQKRLKGAGQSSPATGSQNQSGNTGSIINNYYMQQYQNSMDTQLGDNAISGGSNEGSTDTTSTHTTNTQNNDWFSKLASSAFSGLFGALLADKKTEETTLLEDRILTTRNGHTTSTTQSSVGVTYGYSTQEDHVSGPNTSGLETRVVQAERFFKKYLFDWTPDKAFGHLEKLELPTDHKGVYGHLVDSFAYMRNGWDVEVSAVGNQFNGGCLLVAMVPEWKELTPREKYQLTLFPHQFISPRTNMTAHIVVPYLGVNRYDQYKKHKPWTLVVMVVSPLTTNTVSAGQIKVYANIAPTHVHVAGELPSKEGIVPVACSDGYGGLVTTDPKTADPVYGMVYNPPRTNYPGRFTNLLDVAEACPTFLCFDDGKPYVVTRTDEQRLLAKFDLSLAAKHMSNTYLSGIAQYYAQYSGTINLHFMFTGSTDSKARYMVAYVPPGVETPPDTPEKAAHCIHAEWDTGLNSKFTFSIPYVSAADYAYTASDVAETTNVQGWVCIYQITHGKAEQDTLVVSVSAGKDFELRLPIDPRSQTTSTGESADPVTTTVENYGGETQVQRRQHTDVTFIMDRFVKIQNLNPIHVIDLMQTHQHGLVGALLRAATYYFSDLEIVVRHDGNLTWVPNGAPEAALSNMGNPTAYPKAPFTRLALPYTAPHRVLATVYNGTGKYSAGGMGRRGDLEPLAARVAAQLPTSFNFGAIQATTIHELLVRMKRAELYCPRPLLAVEVSSQDRHKQKIIAPAKQLLNFDLLKLAGDVESNPGPFFFSDVRSNFSKLVETINQMQEDMSTKHGPDFNRLVSAFEELATGVKAIRTGLDEAKPWYKLIKLLSRLSCMAAVAARSKDPVLVAIMLADTGLEILDSTFVVKKISDSLSSLFHVPAPVFSFGAPILLAGLVKVASSFFRSTPEDLERAEKQLKARDINDIFAILKNGEWLVKLILAIRDWIKAWIASEEKFVTMTDLVPGILEKQRDLNDPSKYKEAKEWLDSARQACLKNGNVHIANLCKVVTPAPSKSRPEPVVVCLRGKSGQGKSFLANVLAQAISTHFTGRIDSVWYCPPDPDHFDGYNQQTVVVMDDLGQNPDGKDFKYFAQMVSTTGFIPPMASLEDKGKPFNSKVIITTTNLYSGFTPRTMVCPDALNRRFHFDIDVSAKDGYKVNNKLDITKALEDTHTNPVAMFKYDCALLNGMAVEMKRMQQDMFKPQPPLQNVYQLVQEVIERVELHEKVSSHQIFKQISIPSQKSVLYFLIEKGQHEAAIEFFEGLVHDSIKEELRPLIQQTSFVKRAFKRLKENFEIVALCLTLLANIVIMIRETRKRQQMVDDAVNEYIEKANITTDDKTLDEAEKNPLETSGVSIVGFRERTLPGHRASDDVNSEPARPVEEQPQAEGPYTGPLERQKPLKVKAKLPQQEGPYAGPMERQKPLKVKVKAPVVKEGPYEGPVKKPVALKVKAKNLIVTESGAPPTDLQKMVMGNTKPVELILDGKTVAICCATGVFGTAYLVPRHLFAEKYDKIMLDGRAMTDSDYRVFEFEIKVKGQDMLSDAALMVLHRGNRVRDITKHFRDTARMKKGTPVVGVINNADVGRLIFSGEALTYKDIVVCMDGDTMPGLFAYKAATKAGYCGGAVLAKDGADTFIVGTHSAGGNGVGYCSCVSRSMLLKMKAHIDPEPHHEGLIVDTRDVEERVHVMRKTKLAPTVAHGVFNPEFGPAALSNKDPRLNEGVVLDEVIFSKHKGDTKMTEEDKALFRRCAADYASRLHNVLGTANAPLSIYEAIKGVDGLDAMEPDTAPGLPWALQGKRRGTLIDFENGTVGPEVASALELMEKRQYKFTCQTFLKDEVRPMEKVRAGKTRIVDVLPVEHILYTRMMIGRFCAQMHSNNGPQIGSAVGCNPDVDWQRFGTHFAQYKNVWDVDYSAFDANHCSDAMNIMFEEVFRTEFGFHPNAEWILKTLVNTEHAYENKRITVEGGMPSGCSATSIINTILNNIYVLYALRRHYEGVELDTYTMISYGDDIVVASDYDLDFEALKPHFKSLGQTITPADKSDKGFVLGQSITDVTFLKRHFRMDYGTGFYKPVMASKTLEAILSFARRGTIQEKLISVAGLAVHSGPDEYRRLFEPFQGLFEIPSYRSLYLRWVNAVCGDAQSL

SEQ ID NO: 159 - protein, 2157 aa, synthetic construct
  MOD_RES 1378: note = XXXXX
  MOD_RES 1423: note = XXXXX
  MOD_RES 1430: note = XXXXX
MGAQVSRQNVGTHSTQNSVSNGSSLNYFNINYFKDAASSGASRLDFSQDPSKFTDPVKDVLEKGIPTLQSPSVEACGYSDRIMQITRGDSTITSQDVANAVVGYGVWPHYLTPQDATAIDKPTQPDTSSNRFYTLESKHWNGSSKGWWWKLPDALKDMGIFGENMYYHFLGRSGYTVHVQCNASKFHQGTLLVAMIPEHQLASAKHGSVTAGYKLTHPGEAGRDVSQERDASLRQPSDDSWLNFDGTLLGNLLIFPHQFINLRSNNSATLIVPYVNAVPMDSMLRHNNWSLVIIPISPLRSETTSSNIVPITVSISPMCAEFSGARAKNIKQGLPVYITPGSGQFMTTDDMQSPCALPWYHPTKEISIPGEVKNLIEMCQVDTLIPVNNVGNNVGNVSMYTVQLGNQTGMAQKVFSIKVDITSQPLATTLIGEIASYYTHWTGSLRFSFMFCGTANTTLKLLLAYTPPGIDEPTTRKDAMLGTHVVWDVGLQSTISLVVPWVSASHFRLTADNKYSMAGYITCWYQTNLVVPPSTPQTADMLCFVSACKDFCLRMARDTDLHIQSGPIEQNPVENYIDEVLNEVLVVPNIKESHHTTSNSAPLLDAAETGHTSNVQPEDAIETRYVITSQTRDEMSIESFLGRSGCVHISRIKVDYTDYNGQDINFTKWKITLQEMAQIRRKFELFTYVRFDSEITLVPCIAGRGDDIGHIVMQYMYVPPGAPIPSKRNDFSWQSGTNMSIFWQHGQPFPRFSLPFLSIASAYYMFYDGYDGDNTSSKYGSVVTNDMGTICSRIVTEKQKHSVVITTHIYHKAKHTKAWCPRPPRAVPYTHSHVTNYMPETGDVTTAIVRRNTITTAGPSDLYVHVGNLIYRNLHLFNSEMHDSILISYSSDLIIYRTNTIGDDYIPNCNCTEATYYCRHKNRYYPIKVTPHDWYEIQESEYYPKHIQYNLLIGEGPCEPGDCGGKLLCRHGVIGIITAGGEGHVAFIDLRQFHCAEEQGITDYIHMLGEAFGNGFVDSVKEQINAINPINNISKKVIKWLLRIISAMVIIIRNSSDPQTIIATLTLIGCNGSPWRFLKEKFCKWTQLTYIHKESDSWLKKFTEMCNAARGLEWIGNKISKFIDWMKSMLPQAQLKVKYLNEIKKLSLLEKQIENLRAADSATQEKIKCEIDTLHDLSCKFLPLYAHEAKRIKVLYNKCSNIIKQRKRSEPVAVMIHGPPGTGKSITTNFLARMITNESDVYSLPPDPKYFDGYDNQSVVIMDDIMQNPDGEDMTLFCQMVSSVTFIPPMADLPDKGKPFDSRFILCSTNHSLLAPPTISSLPAMNRRFFFDLDIVVHDNYKDTQGKLDVSKAFRPCNVNTKIGNAKCCPFVCGKAVXFKDRSTCSTYTLAQVYNHILEEDKRRRQVVDVMSAIFQGPISLDXPPPPAIXDLLQSVRTPEVIKYCQDNKWVIPAECQVERDLNIANSIIAIIANIISIAGIIFVIYKLFCSLQGPYSGEPKPKTKVPERRVVAQGPEEEFGRSILKNNTCVITTGNGKFTGLGIHDRILIIPTHADPGREVQVNGVHTKVLDSYDLYNRDGVKLEITVIQLDRNEKFRDIRKYIPETEDDYPECNLALSANQDEPTIIKVGDVVSYGNILLSGNQTARMLKYNYPTKSGYCGGVLYKIGQILGIHVGGNGRDGFSAMLLRSYFTDTQGQIKVNKHATECGLPTIHTPSKTKLQPSVFYDVFPGSKEPAVLTDNDPRLEVNFKEALFSKYKGNVECNLNEHMEIAIAHYSAQLMTLDIDSRPIALEDSVFGIEGLEALDLNTSAGFPYVTMGIKKRDLINNKTKDISRLKEALDKYGVDLPMITFLKDELRKKEKISTGKTRVIEASSINDTILFRTTFGNLFSKFHLNPGVVTGSAVGCDPETFWSKIPVMLDGDCIMAFDYTNYDGSIHPVWFQALKKVLENLSFQSNLIDRLCYSKHLFKSTYYEVAGGVPSGCSGTSIFNTMINNIIIRTLVLDAYKNIDLDKLKIIAYGDDVIFSYKYTLDMEAIANEGKKYGLTITPADKSNEFKKLDYSNVTFLKRGFKQDERHTFLIHPTFPVEEIHESIRWTKKPSQMQEHVLSLCHLMWHNGRKVYEDFSSKIRSVSAGRALYIPPYDLLKHEWYEKF

SEQ ID NO: 160 - protein, 2153 aa, synthetic construct
MGAQVSRQNVGTHSTQNMVSNGSSLNYFNINYFKDAASSGASRLDFSQDPSKFTDPVKDVLEKGIPTLQSPSVEACGYSDRIIQITRGDSTITSQDVANAVVGYGVWPHYLTPQDATAIDKPTQPDTSSNRFYTLDSKMWNSTSKGWWWKLPDALKDMGIFGENMFYHFLGRSGYTVHVQCNASKFHQGTLLVVMIPEHQLATVNKGNVNAGYKYTHPGEAGREVGTQVENEKQPSDDNWLNFDGTLLGNLLIFPHQFINLRSNNSATLIVPYVNAVPMDSMVRHNNWSLVIIPVCQLQSNNISNIVPITVSISPMCAEFSGARAKTVVQGLPVYVTPGSGQFMTTDDMQSPCALPWYHPTKEIFIPGEVKNLIEMCQVDTLIPINSTQSNIGNVSMYTVTLSPQTKLAEEIFAIKVDIASHPLATTLIGEIASYFTHWTGSLRFSFMFCGTANTTLKVLLAYTPPGIGKPRSRKEAMLGTHVVWDVGLQSTVSLVVPWISASQYRFTTPDTYSSAGYITCWYQTNFVVPPNTPNTAEMLCFVSGCKDFCLRMARDTDLHKQTGPITQNPVERYVDEVLNEVLVVPNINQSHPTTSNAAPVLDAAETGHTNKIQPEDTIETRYVQSSQTLDEMSVESFLGRSGCIHESVLDIVDNYNDQSFTKWNINLQEMAQIRRKFEMFTYARFDSEITMVPSVAAKDGHIGHIVMQYMYVPPGAPIPTTRDDYAWQSGTNASVFWQHGQPFPRFSLPFLSIASAYYMFYDGYDGDTYKSRYGTVVTNDMGTLCSRIVTSEQLHKVKVVTRIYHKAKHTKAWCPRPPRAVQYSHTHTTNYKLSSEVHNDVAIRPRTNLTTVGPSDMYVHVGNLIYRNLHLFNSDIHDSILVSYSSDLIIYRTSTQGDGYIPTCNCTEATYYCKHKNRYYPINVTPHDWYEIQESEYYPKHIQYNLLIGEGPCEPGDCGGKLLCKHGVIGIITAGGEGHVAFIDLRHFHCAEEQGITDYIHMLGEAFGSGFVDSVKDQINSINPINNISSKMVKWMLRIISAMVIIIRNSSDPQTIIATLTLIGCNGSPWRFLKEKFCKWTQLTYIHKESDSWLKKFTEMCNAARGLEWIGNKISKFIDWMKSMLPQAQLKVKYLSELKKLNFLEKQVENLRAADTNTQEKIKCEIDTLHDLSCKFLPLYASEAKRIKVLYHKCTNIIKQKKRSEPVAVMIHGPPGTGKSITTSFLARMITNESDIYSLPPDPKYFDGYDNQSVVIMDDIMQNPGGEDMTLFCQMVSSVTFIPPMADLPDKGKPFDSRFVLCSTNHSLLAPPTISSLPAMNRRFYLDLDILVHDNYKDNQGKLDVSRAFRLCDVDSKIGNAKCCPFVCGKAVTFKDRNTCRTYSLSQIYNQILEEDKRRRQVVDVMSAIFQGPISMDKPPPPAITDLLRSVRTPEVIKYCQDNKWIVPADCQIERDLNIANSIITIIANIISIAGIIYIIYKLFCSLQGPYSGEPKPKTKVPERRVVAQGPEEEFGMSIIKNNTCVVTTTNGKFTGLGIYDRILILPTHADPGSEIQVNGIHTKVLDSYDLFNKEGVKLEITVLKLDRNEKFRDIRKYIPESEDDYPECNLALVANQTEPTIIKVGDVVSYGNILLSGTQTARMLKYNYPTKSGYCGGVLYKIGQILGIHVGGNGRDGFSSMLLRSYFTEQQGQIQISKHVKDVGLPSIHTPTKTKLQPSVFYDIFPGSKEPAVLTEKDPRLKVDFDSALFSKYKGNTECSLNEHIQVAVAHYSAQLATLDIDPQPIAMEDSVFGMDGLEALDLNTSAGYPYVTLGIKKKDLINNKTKDISKLKLALDKYDVDLPMITFLKDELRKKDKIAAGKTRVIEASSINDTILFRTVYGNLFSKFHLNPGVVTGCAVGCDPETFWSKIPLMLDGDCIMAFDYTNYDGSIHPIWFKALGMVLDNLSFNPTLINRLCNSKHIFKSTYYEVEGGVPSGCSGTSIFNSMINNIIIRTLVLDAYKHIDLDKLKIIAYGDDVIFSYKYKLDMEAIAKEGQKYGLTITPADKSSEFKELDYGNVTFLKRGFRQDDKYKFLIHPTFPVEEIYESIRWTKKPSQMQEHVLSLCHLMWHNGPEIYKDFETKIRSVSAGRALYIPPYELLRHEWYEKF

SEQ ID NO: 161 - protein, 2150 aa, synthetic construct
MGAQVSRQNVGTHSTQNSVSNGSSLNYFNINYFKDAASNGASKLEFTQDPSKFTDPVKDVLEKGIPTLQSPTVEACGYSDRIIQITRGDSTITSQDVANAIVAYGVWPHYLSSKDASAIDKPSQPDTSSNRFYTLRSVTWSSSSKGWWWKLPDALKDMGIFGENMFYHYLGRSGYTIHVQCNASKFHQGTLIVALIPEHQIASALHGNVNVGYNYTHPGETGREVKAETRLNPDLQPTEEYWLNFDGTLLGNITIFPHQFINLRSNNSATIIAPYVNAVPMDSMRSHNNWSLVIIPICPLETSSAINTIPITISISPMCAEFSGARAKRQGLPVFITPGSGQFLTTDDFQSPCALPWYHPTKEISIPGEVKNLVEICQVDSLVPINNTDTYINSENMYSVVLQSSINAPDKIFSIRTDVASQPLATTLIGEISSYFTHWTGSLRFSFMFCGTANTTVKLLLAYTPPGIAEPTTRKDAMLGTHVIWDVGLQSTISMVVPWISASHYRNTSPGRSTSGYITCWYQTRLVIPPQTPPTARLLCFVSGCKDFCLRMARDTNLHLQSGAIAQNPVENYIDEVLNEVLVVPNINSSNPTTSNSAPALDAAETGHTSSVQPEDVIETRYVQTSQTRDEMSLESFLGRSGCIHESKLEVTLANYNKENFTVWAINLQEMAQIRRKFELFTYTRFDSEITLVPCISALSQDIGHITMQYMYVPPGAPVPNSRDDYAWQSGTNASVFWQHGQAYPRFSLPFLSVASAYYMFYDGYDEQDQNYGTANTNNMGSLCSRIVTEKHIHKVHIMTRIYHKAKHVKAWCPRPPRALEYTRAHRTNFKIEDRSIQTAIVTRPIITTAGPSDMYVHVGNLIYRNLHLFNSEMHESILVSYSSDLIIYRTNTVGDDYIPSCDCTQATYYCKHKNRYFPITVTSHDWYEIQESEYYPKHIQYNLLIGEGPCEPGDCGGKLLCKHGVIGIVTAGGDNHVAFIDLRHFHCAEEQGVTDYIHMLGEAFGNGFVDSVKEHIHAINPVGNISKKIIKWMLRIISAMVIIIRNSSDPQTILATLTLIGCSGSPWRFLKEKFCKWTQLNYIHKESDSWLKKFTEACNAARGLEWIGNKISKFIEWMKSMLPQAQLKVKYLNELKKLNLYEKQVESLRVADMKTQEKIKMEIDTLHDLSRKFLPLYASEAKRIKTLYIKCDNIIKQKKRCEPVAIVIHGPPGAGKSITTNFLAKMITNDSDIYSLPPDPKYFDGYDQQSVVIMDDIMQNPAGDDMTLFCQMVSSVTFIPPMADLPDKGKAFDSRFVLCSTNHSLLTPPTITSLPAMNRRFFLDLDIIVHDNFKDPQGKLNVAAAFRPCDVDNRIGNARCCPFVCGKAVSFKDRNSCNKYSLAQVYNIMIEEDRRRRQVVDVMTAIFQGPIDMKNPPPPAITDLLQSVRTPEVIKYCEGNRWIIPAECKIEKELNLANTIITIIANVIGMARIIYVIYKLFCTLQGPYSGEPKPKTKIPERRVVTQGPEEEFGMSLIKHNSCVITTENGKFTGLGVYDRFVVVPTHADPGKEIQVDGITTKVIDSYDLYNKNGIKLEITVLKLDRNEKFRDIRRYIPNNEDDYPNCNLALLANQPEPTIINVGDVVSYGNILLSGNQTARMLKYSYPTKSGYCGGVLYKIGQVLGIHVGGNGRDGFSAMLLRSYFTDVQGQITLSKKTSECNLPSIHTPCKTKLQPSVFYDVFPGSKEPAVLSEKDARLQVDFNEALFSKYKGNTDCSINDHIRIASSHYAAQLITLDIDPKPITLEDSVFGTDGLEALDLNTSAGFPYIAMGVKKRDLINNKTKDISKLKEAIDKYGVDLPMVTFLKDELRKHEKVIKGKTRVIEASSVNDTLLFRTTFGNLFSKFHLNPGIVTGSAVGCDPEVFWSKIPAMLDDKCIMAFDYTNYDGSIHPIWFEALKQVLVDLSFNPTLIDRLCKSKHIFKNTYYEVEGGVPSGCSGTSIFNTMINNIIIRTLVLDAYKNIDLDKLKIIAYGDDVIFSYIHELDMEAIAIEGVKYGLTITPADKSNTFVKLDYSNVTFLKRGFKQDEKYNFLIHPTFPEDEIFESIRWTKKPSQMHEHVLSLCHLMWHNGRDAYKKFVEKIRSVSAGRALYIPPYDLLLHEWYEKF

SEQ ID NO: 162 - protein, 2164 aa, synthetic construct
MGAQVSRQNVGTHSTQNSVSNGSSLNYFNINYFKDAASSGASRLDFSQDPSKFTDPVKDVLEKGIPTLQSPTVEACGYSDRLIQITRGDSTITSQDTANAVVAYGVWPSYLTPDDATAIDKPTQPDTSSNRFYTLDSRSWTSASSGWWWKLPDALKNMGIFGENMFYHFLGRSGYTIHVQCNSSKFHQGLLIVAAIPEHQLASATSGNVSVGYNHTHPGEQGREVVPSRTSSDNKRPSDDSWLNFDGTLLGNLPIYPHQYINLRTNNSATLILPYVNAVPMDSMLRHNNWSLVIIPICPLQVQPGGTQSIPITVSISPMFSEFSGPRSKVVFSTTQGLPVMLTPGSGQFLTTDDTQSPSAFPYFHPTKEIFIPGQVRNLIEMCQVDTLIPVNNTQENVRSVNMYTVDLRTQVDLAKEVFSIPVDIASQPLATTLIGELASYYTHWTGSLRFSFMFCGSASSTLKLLIAYTPPGVGKPKSRREAMLGTHLVWDVGLQSTASLVVPWVSASHFRFTTPDTYSSAGYITCWYQTNFVVPDSTPDNAKMVCMVSACKDFCLRLARDTNLHTQEGVLTQNPVENYIDSVLNEVLVVPNIQPSTSVSSHAAPALDAAETGHTSSVQPEDMIETRYVITDQTRDETSIESFLGRSGCIAMIEFNTSSDKTEHDKIGKGFKTWKVSLQEMAQIRRKYELFTYTRFDSEITIVTAAAAQGNDSGHIVLQFMYVPPGAPVPEKRDDYTWQSGTNASVFWQEGQPYPRFTIPFMSIASAYYMFYDGYDGDSAASKYGSVVTNDMGTICVRIVTSNQKHDSNIVCRIYHKAKHIKAWCPRPPRAVAYQHTHSTNYIPSNGEATTQIKTRPDVFTVTNVGPSSMFVHVGNLIYRNLHLFNSDLDDSILVSYSSDLIIYRTNTEGNDVIPNCDCTECTYYCHHKDRYFPIRVTAHDWYEIQESEYYPKHIQYNLLIGEGPCEPGDCGGKLLCKHGVIGMITAGGEGHVAFIDLRKFQCAEEQGLSDYVEHLGQVFGVGFVDSIKQQVNFINPTSKIGSKVIKWLLRIVSAMIIMVRNSSDPQTVIATLTLLGCSGSPWRFLKEKLCAWLQLSYVHKQSDSWLKKFTEACNAARGLEWIGQKISKFIDWIKSMLPQAQLKIDYLTKLKQLNLLEKQIETIRLAPASVQEKIFIEINTLHDLSLKFLPLYASEARRIKNLYIKCSNVIKGGKRNEPVAVLIHGSPGTGKSLATSVLARMLTVETDIYSLPPDPKYFDGYDQQSVVIMDDIMQNPSGEDMTLFCQMVSSVPFIPPMADLPDKGKPFTSKFVLASTNHTLLTPPTVSSLPAMARRFYFDLDIQVKKEYLLDGKLDIAKSFRPCDVNIKIGNAKCCPFICGKAVEFKDRNSCTTLSLSQLYSHIKEEDRRRSSAAQAMEAIFQGIDLQSPPPPAIADLLRSVKTPEIIKYCQDNNWIVPAECSIERDLGIANMTIGIIANVVSIVGVIYIIYKLFCTLQGPYSGEPKPKSRAPERRVVTQGPEEEFGRSLLKHNCCVVTTDKGKFTGLGIYDQVMVLPTHSDPGSEILVDGVKVKVSDSYDLHNHEGVKLEITVVKLIRNEKFKDIRKYLPSREDDYPACNLALLANQDEPTIISVGDAVSYGNILLSGTNTARMIKYHYPTKAGYCGGVLYKVGSILGIHVGGNGRDGFSAMLLKSYFGETQGLITKELPVSVKNLPSVHVSSKTRLQPSVFHDVFPGTKEPAVLSSNDPRLETDFDSALFSKYKGNPACQVTPHMKIAVAHYAAQLSTLDINPQPLSLEESVFGIEGLEALDLNTSAGFPYVSLGIKKKDLIDKKTKDITKLRKAIDEYGIDLPMVTFLKDELRKKEKIKDGKTRVIEANSVNDTVLFRSVFGNLFSAFHKNPGIVTGSAVGCDPEVFWSTIPLMLDGECLMAFDYSNYDGSLHPVWFKCLSMLLEDIGFSSQLINQICNSKHIYKSKYYEVEGGMPSGCAGTSIFNTIINNIIIRTLVLDAYKNIDLDKLKILAYGDDVIFSYNFKLDMAVLAKEGEKYGLTITPADKSDVFQELTYKNVTFLKRGFRADERHSFLIHPTFPVAEIHDSIRWTKNPSCMQEHVLSLCHLMWHNGRHAYQEFIKGIRSVSAGRALYIPAYEVLEHEWYEKF

SEQ ID NO: 163 - protein, 2179 aa, synthetic construct
MGAQVSTQKSGSHENQNILTNGSNQTFTVINYYKDAASTSSAGQSLSMDPSKFTEPVKDLMLKGAPALNSPNVEACGYSDRVQQITLGNSTITTQEAANAVVCYAEWPEYLPDVDASDVNKTSKPDTSVCRFYTLDSKTWTTGSKGWCWKLPDALKDMGVFGQNMFFHSLGRSGYTVHVQCNATKFHSGCLLVVVIPEHQLASHEGGNVSVKYTFTHPGERGIDLSSANEVGGPVKDVIYNMNGTLLGNLLIFPHQFINLRTNNTATIVIPYINSVPIDSMTRHNNVSLMVIPIAPLTVPTGATPSLPITVTIAPMCTEFSGIRSKSIVPQGLPTTTLPGSGQFLTTDDRQSPSALPNYEPTPRIHIPGKVHNLLEIIQVDTLIPMNNTHTKDEVNSYLIPLNANRQNEQVFGTNLFIGDGVFKTTLLGEIVQYYTHWSGSLRFSLMYTGPALSSAKLILAYTPPGARGPQDRREAMLGTHVVWDIGLQSTIVMTIPWTSGVQFRYTDPDTYTSAGFLSCWYQTSLILPPETTGQVYLLSFISACPDFKLRLMKDTQTISQTVALTEGLGDELEEVIVEKTKQTVASISSGPKHTQKVPILTANETGATMPVLPSDSIETRTTYMHFNGSETDVECFLGRAACVHVTEIQNKDATGIDNHREAKLFNDWKINLSSLVQLRKKLELFTYVRFDSEYTILATASQPDSANYSSNLVVQAMYVPPGAPNPKEWDDYTWQSASNPSVFFKVGDTSRFSVPYVGLASAYNCFYDGYSHDDAETQYGITVLNHMGSMAFRIVNEHDEHKTLVKIRVYHRAKHVEAWIPRAPRALPYTSIGRTNYPKNTEPVIKKRKGDIKSYGLGPRYGGIYTSNVKIMNYHLMTPEDHHNLIAPYPNRDLAIVSTGGHGAETIPHCNCTSGVYYSTYYRKYYPIICEKPTNIWIEGNPYYPSRFQAGVMKGVGPAEPGDCGGILRCIHGPIGLLTAGGSGYVCFADIRQLECIAEEQGLSDYITGLGRAFGVGFTDQISTKVTELQEVAKDFLTTKVLSKVVKMVSALVIICRNHDDLVTVTATLALLGCDGSPWRFLKMYISKHFQVPYIERQANDGWFRKFNDACNAAKGLEWIANKISKLIEWIKNKVLPQAKEKLEFCSKLKQLDILERQITTMHISNPTQEKREQLFNNVLWLEQMSQKFAPLYAVESKRIRELKNKMVNYMQFKSKQRIEPVCVLIHGTPGSGKSLTTSIVGRAIAEHFNSAVYSLPPDPKHFDGYQQQEVVIMDDLNQNPDGQDISMFCQMVSSVDFLPPMASLDNKGMLFTSNFVLASTNSNTLSPPTILNPEALVRRFGFDLDICLHTTYTKNGKLNAGMSTKTCKDCHQPSNFKKCCPLVCGKAISLVDRTTNIRYSVDQLVTAIISDFKSKMQITDSLETLFQGPVYKDLEIDVCNTPPPECINDLLKSVDSEEIREYCKKKKWIIPEIPTNIERAMNQASMIINTILMFVSTLGIVYVIYKLFAQTQGPYSGNPPHNKLKAPTLRPVVVQGPNTEFALSLLRKNIMTITTSKGEFTGLGIHDRVCVIPTHAQPGDDVLVNGQKIRVKDKYKLVDPENINLELTVLTLDRNEKFRDIRGFISEDLEGVDATLVVHSNNFTNTILEVGPVTMAGLINLSSTPTNRMIRYDYATKTGQCGGVLCATGKIFGIHVGGNGRQGFSAQLKKQYFVEKQGQVIARHKVREFNINPVNTPTKSKLHPSVFYDVFPGDKEPAVLSDNDPRLEVKLTESLFSKYKGNVNTEPTENMLVAVDHYAGQLLSLDIPTSELTLKEALYGVDGLEPIDITTSAGFPYVSLGIKKRDILNKETQDTEKMKFYLDKYGIDLPLVTYIKDELRSVDKVRLGKSRLIEASSLNDSVNMRMKLGNLYKAFHQNPGVLTGSAVGCDPDVFWSVIPCLMDGHLMAFDYSNFDASLSPVWFVCLEKVLTKLGFAGSSLIQSICNTHHIFRDEIYVVEGGMPSGCSGTSIFNSMINNIIIRTLILDAYKGIDLDKLKILAYGDDLIVSYPYELDPQVLATLGKNYGLTITPPDKSETFTKMTWENLTFLKRYFKPDQQFPFLVHPVMPMKDIHESIRWTKDPKNTQDHVRSLCMLAWHSGEKEYNEFIQKIRTTDIGKCLILPEYSVLRRRWLDLF

SEQ ID NO: 164 - protein, 2178 aa, synthetic construct
MGAQVSTQKSGSHENQNILTNGSNQTFTVINYYKDAASSSSAGQSFSMDPSKFTEPVKDLMLKGAPALNSPNVEACGYSDRVQQITLGNSTITTQEAANAIVCYAEWPEYLSDNDASDVNKTSKPDISVCRFYTLDSKTWKATSKGWCWKLPDALKDMGVFGQNMFYHSLGRTGYTIHVQCNATKFHSGCLLVVVIPEHQLASHEGGTVSVKYKYTHPGDRGIDLDTVEVAGGPTSDAIYNMDGTLLGNLLIFPHQFINLRTNNTATIVVPYINSVPIDSMTRHNNVSLMVVPIAPLNAPTGSSPTLPVTVTIAPMCTEFTGIRSRSIVPQGLPTTTLPGSGQFLTTDDRQSPSALPSYEPTPRIHIPGKVRNLLEIIQVGTLIPMNNTGTNDNVTNYLIPLHADRQNEQIFGTKLYIGDGVFKTTLLGEIAQYYTHWSGSLRISLMYTGPALSSAKIILAYTPPGTRGPEDRKEAMLGTHVVWDIGLQSTIVMTIPWTSGVQFRYTDPDTYTSAGYLSCWYQTSLILPPQTSGQVYLLSFISACPDFKLRLMKDTQTISQTDALTEGLSDELEEVIVEKTKQTLASVSSGPKHTQSVPALTANETGATLPTRPSDNVETRTTYMHFNGSETDVESFLGRAACVHVTEIKNKNAAGLDNHRKEGLFNDWKINLSSLVQLRKKLELFTYVRFDSEYTILATASQPEASSYSSNLTVQAMYVPPGAPNPKEWDDYTWQSASNPSVFFKVGETSRFSVPFVGIASAYNCFYDGYSHDDPDTPYGITVLNHMGSMAFRVVNEHDVHTTIVKIRVYHRAKHVEAWIPRAPRALPYVSIGRTNYPRDSKTIIKKRTNIKTYGLGPRFGGVFTSNVKIINYHLMTPDDHLNLVAPYPNRDLAVVATGAHGAETIPHCNCTSGVYYSRYYRKFYPIICERPTNIWIEGSSYYPSRYQAGVMKGVGPAEPGDCGGILRCIHGPIGLLTAGGGGYVCFADIRQLDFIADEQGLGDYITSLGRAFGTGFTDQISAKVCELQDVAKDFLTTKVLSKVVKMISALVIICRNHDDLVTVTATLALLGCDGSPWRFLKMYISKHFQVPYIERQANDGWFRKFNDACNAAKGLEWIANKISKLIEWIKNKVLPQAREKLEFCSKLKQLDILERQIASIHDSNPTQEKREQLFNNVLWLEQMSQKFSPLYASEAKRIRDLKNKITNYMQFKSKQRTEPVCVLIHGTPGSGKSLTTSIVGRALAEHFNSSVYSLPPDPKHFDGYQQQEVVIMDDLNQNPDGQDISMFCQMVSSVDFLPPMASLDNKGMLFTSNFVLASTNSNTLSPPTILNPEALIRRFGFDLDICMHSTYTKNGKLNAAMATSLCKDCHQPSNFKKCCPLVCGKAISLVDRVSNVRFSIDQLVTAIINDYKNKVKITDSLEVLFQGPVYKDLEIDICNTPPPECISDLLKSVDSEEVREYCKKKKWIIPQISTNIERAVNQASMIINTILMFVSTLGIVYVIYKLFAQTQGPYSGNPVHNKLKPPTLKPVVVQGPNTEFALSLLRKNILTITTEKGEFTSLGIHDRICVLPTHAQPGDNVLVNGQKIQIKDKYKLVDPDNTNLELTIIELDRNEKFRDIRGFISEDLEGLDATLVVHSNGFTNTILDVGPITMAGLINLSNTPTTRMIRYDYPTKTGQCGGVLCTTGKIFGIHVGGNGRRGFSAQLKKQYFVEKQGLIVSKQKVRDIGLNPINTPTKTKLHPSVFYNVFPGSKQPAVLNDNDPRLEVKLAESLFSKYKGNVQMEPTENMLIAVDHYAGQLMSLDISTKELTLKEALYGVDGLEPIDVTTSAGYPYVSLGIKKRDILNKETQDVEKMKFYLDKYGIDLPLVTYIKDELRSVDKVRLGKSRLIEASSLNDSVNMRMKLGNLYKAFHQNPGIITESAVGCDPDVFWSVIPCLMDGHLMAFDYSNFDASLSPVWFECLEKVLNKLGFKQPSLIQSICNTHHIFRDEIYRVEGGMPSGCSGTSIFNSMINNIIIRTLILDAYKGIDLDSLRILAYGDDLIVSYPFELDSNILAAIGKNYGLTITPPDKSDAFTKITWENITFLKRYFRPDPQFPFLIHPVMPMQDIYESIRWTRDPRNTQDHVRSLCMLAWHSGEKDYNDFITKIRTTDIGKCLNLPEYSVLRRRWLDLF

SEQ ID NO: 165 - protein, 2175 aa, synthetic construct
MGAQLSRNTAGSHTTGTYATGGSTINYNNINYYSHAASAAQNKQDFTQDPSKFTQPIADVIKETAVPLKSPSAEACGYSDRVAQLTLGNSTITTQEAANICVAYGCWPAKLSDTDATSVDKPTEPGVSADAFYTLRSKPWQADSKGWYWKLPDALNNTGMFGQNAQFHYIYRGGWAVHVQCNATKFHQGTLLVLAIPEHQIATQEQPAFDRTMPGSEGGTFQEPFWLEDGTSLGNSLIYPHQWINLRTNNSATLILPYVNAIPMDSAIRHSNWTLAIIPVAPLKYAAETTPLVPITVTIAPMETEYNGLRRAIASNQGLPTKPGPGSYQFMTTDEDCSPCILPDFQPTLEIFIPGKVNNLLEIAQVESILEANNREGVEGVERYVIPVSVQDALDAQIYALRLELGGSGPLSSSLLGTLAKHYTQWSGSVEITCMFTGTFMTTGKVLLAYTPPGGDMPRNREEAMLGTHVVWDFGLQSSITLVIPWISASHFRGVSNDDVLNYQYYAAGHVTIWYQTNMVIPPGFPNTAGIIMMIAAQPNFSFRIQKDREDMTQTAILQNDPGKMLKDAIDKQVAGALVAGTTTSTHSVATDSTPALQAAETGATSTARDESMIETRTIVPTHGIHETSVESFFGRSSLVGMPLLATGTSITNWRIDFREFVQLRAKMSWFTYMRFDVEFTIIATSSTGQNVTTEQHTTYQVMYVPPGAPVPSNQDSFQWQSGCNPSVFADTDGPPAQFSVPFMSSANAYSTVYDGYARFMDTDPDRYGILPSNFLGFMYFRTLEDAAHQVRFRICAKIKHTSCWIPRAPRQAPYKKRYNLVFSGDSDRICSNRASLTSYGPFGQQQGAAYVGSYKILNRHLATYADWENEVWQSYQRDLLVTRVDAHGCDTIARCNCRSGIYYCKSTAKHYPIVVTPPSIYKIEANDYYPERMQTHILLGIGFAEPGDCGGLLRCEHGVMGILTVGGGDHVGFADVRDLLWIEDDAMEQGITDYVQQLGNAFGAGFTAEIANYTNQLRDMLMGSDSVVEKIIRSLVRLVSALVIVVRNHQDLITVGATLALLGCEGSPWKWLKRKVCQILGINMAERQSDNWMKKFTEMCNAFRGLDWIAAKISKFIDWLKQKILPELKERAEFVKKLKQLPLLEAQVNTLEHSSASQERQEQLFGNVQYLAHHCRKNAPLYAAEAKRVYHLEKRVLGAMQFKTKNRIEPVCALIHGSPGTGQSLATMIVGRKLAEYEGSDVYSLPPDPDHFDGYQQQAVVVMDDLLQNPDGKDMTLFCQMVSTAPFTVPMAALEDKGKLFTSKFVLASTNAGQVTPPTVADYKALQRRFFFDCDIEVQKEYKRDGVTLDVAKATETCEDCSPANFKKCMPLICGKALQLKSRKGDGMRYSLDTLISELRRESNRRYNIGNVLEALFQGPVCYKPLRIEVHEEEPAPSAISDLLQAVDSEEVREYCRSKGWIVEERVTELKLERNVNRALAVIQSVSLIAAVAGTIYIVYRLFSGMQGPYSGIGTNYATKKPVVRQVQTQGPLFDFGVSLLKKNIRTVKTGAGEFTALGVYDTVVVLPRHAMPGKTIEMNGKDIEVLDAYDLNDKTDTSLELTIVKLKMNEKFRDIRAMVPDQITDYNEAVVVVNTSYYPQLFTCVGRVKDYGFLNLAGRPTHRVLMYEFPTKAGQCGGVVISMGKIVGVHVGGNGAQGFAASLLRRYFTAEQGQIEYIEKSKDAGYPVINAPTQTKLEPSVFFDVFPGVKEPAVLHKKDKRLETNFEEALFSKYIGNVQRDMPEELLIAIDHYSEQLKMLNIDPRPISMEDAIYGTEGLEALDLGTSASYPYVAMGIKKRDILNKETRDVTKMQECIDKYGLNLPMVTYVKDELRAPDKIRKGKSRLIEASSLNDSVAMRCYFGNLYKVFHTNPGTISGCAVGCDPETFWSKIPVMMDGELFGFDYTAYDASLSPMWFHALAEVLRRIGFVECKHFIDQLCCSHHLYMDKHYYVVGGMPSGCSGTSIFNSMINNLIIRTLVLTVYKNIDLDDLKIIAYGDDVLASYPYEIDASLLAEAGKSFGLIMTPPDKSAEFVKLTWDNVTFLKRKFVRDARYPFLVHPVMDMSNIHESIRWTKDPRHTEDHVRSLCLLAWHCGEEEYNEFVTKIRSVPVGRALHLPSFKALERKWYDSF

SEQ ID NO: 166 - protein, 2168 aa, synthetic construct
MGMQMSKNTAGSHTTVTQASGGSHINYTNINYYSHSASASQNKQDITQDPSKFTQPMVDIMKESAVPLKSPSAEACGYSDRVAQLTLGNSTITTQEAANITVAYGEWPSYLSDLDATAVDKTTKPGVSCDRFYTLPGKKWEATTKGWEWKLPDALTELGVFGQNCQFHFLYRCGWSIHVQCNATKFHQGTLLVVAVPDHQLGTTYQPEFDNVMPGKAGREVKYPYNFEDGTSLANSLIYPHQWINLRTNNSATLVLPYANAIPMDSPIRHSSWSLLVIPVVPLACATGTTPFVGITITLAPMFSEFSGLRRAIAQGIPTTNTPGSYQFLTTDEDSSACILPDFTPTQEIHIPGEVKNLQALCQVESLLEINNVDGKTGIERLRLEVSTQSELDRQLFALKVSFTEGEIMSKTLCGVMCSYYTQWSGSLEITFMFTGSFMTTGKLLLAYTPPGGSAPASREDAMLGTHVIWDFGLQSSITLVVPWICGGYYRDVNRANNYYAAGYVTGWFQTNMVIPPDFPSTAYILCFLAAQPNFSLRILKDRPDITQTAALQAPVETALNSAISSVIAGITAQDTQPSSHNISTSETPALQAAETGASSNASDEGMMETRHVVNTNTVSETSIESFYGRCGLVSIKEIADNKQVEKWLVNFNEFVQLRAKIELFTYMRFDIEFTLVATFTKGNSASQHPVQVQVMYLPPEQLLQLQQDSYAWQSAANPSAIFSANTVPARFSVPFVGTANAYTIMYDGYNVFGSNRPSADYGMINSSHMGSMAFRAISQLQATEKVKFMDLCQVKDVRAWCPRAPRMAPYKYIRNPVFETQDRIVPNRNNITTTGAFGQQSGAIYVGNYKIMNRHLATHEDWENVEWEDYNRDILVARTTAHGADKLARCHCNTGVYYCKSRNKHYPVTSRVQASIGSRLVSTTQLDTRPICSLPSGISEPGDCGGILRCQHGVIGIVTAGGQGVVGFADVRDLFWVEHEAMEQGLTDYIQQLGNSFGQGFTAEITNYASQLSEMLIGADGMVERCLQTFVKVISAVVIATRSQGDVPTILATLALIGCDGSPWRWLKRQFCGIFKIPYVEKQGDDWLKKFTSYVNAFKGLDWVAEKIMKFIDWMKNKLIPQARERQEFTTNLKTLPLLEAQVATLEHSCPTTEQQETIFGNIQYLAHHCRRYAPLYAAEARRVYALEKRILGYIQFKSKQRIEPVCLLIHGTAGTGKSLATSIIGRKLAEYEHSEVYAIPPDSDHFDGYQQQAVVVMDDLNQNPDGKDMVAFCQMVSTVPYHVPMAAIEEKGMLFTSSYVLASTNSGSIHPPTVSNSKALSRRFAFDVDIEVSEHYKTHNGTLDVVNATQRCEDCCPANFKTCMPLICGEAYQLVDRRNGMRYSIDTMISAMRAEWKRRNQVGLCYVRLFQGPPQFKPLKISVDPEIPAPPAIADLLASVDSEEVREYCKKKGWIVEVPVTATTLERNVSIATTILSSLVLLTSVITLVYLVYRLFAGYQGPYTGLPNAKPKPPVLREVRAQGPLMDFGVGMMKKNIVTVRTGAGEFTGLGVHDHVLVLPKHSHPAEIVVVDGKETPVEDAYNLTDEQGVSLELTLVTLKRNEKFRDIRAMIPENPCGTNEAVVCVNTSNFPNAFLPVGKVEYYGYLNLAGSPTHRTMMYNFPTKAGQCGGVVLSTGKVLGIHIGGNGAQGFCAALKRSYFTKPQGKIDWVEPSKKHGFPVINAPSKTKLEPSVFFDVFEGVKEPAALHPKDPRLEVNLEEALFSKYTGNVDIEMPEEMKEAVDHYANQLLALDIPTEPLSMEEAIYGTEGLEALDLTTSAGYPYVTMGIKKRDILNKETRDVKKMQECIDKYGLNLPMVTYIKDELRSKEKVKKGKSRLIEASSLNDSVAMRCYFGNLYKAFHQNPGTLTGCAVGCDPDTFWSKIPVMMDGELFGFDYTAYDASLSPLMFQALQMVLEKIGFGEGKHFIDNLCYSHHLFRDKYYFVKGGMPSGCSGTSIFNSMINNIIIRTVVLQTYKGIELDQLKIIAYGDDVIASYPYRIDPAELAKAGAKLGLHMTPPDKSETYVDLDWTNVTFLKRNFVPDEKYPFLVHPVMPMKEIYESIRWTRDARNTQDHVRSLCLLAWHNGRKEYEEFCRKIRSVPVGRALHLPSYSSLLREWYEKF

SEQ ID NO: 167 - protein, 2188 aa, synthetic construct
MGAQVTRQQTGTHENANIATNGSHITYNQINFYKDSYAASASKQDFSQDPSKFTEPVVEGLKAGAPVLKSPSAEACGYSDRVLQLKLGNSAIVTQEAANYCCAYGEWPNYLPDHEAVAIDKPTQPETSTDRFYTLRSVKWESNSTGWWWKLPDALNNIGMFGQNVQYHYLYRSGFLIHVQCNATKFHQGALLVVAIPEHQRGAHDTTTSPGFNDIMKGERGGTFNHPYVLDDGTSIACATIFPHQWINLRTNNSATIVLPWMNVAPMDFPLRHNQWTLAVIPVVPLGTRTMSSVVPITVSIAPMCCEFNGLRHAITQGVPTYLLPGSGQFLTTDDHSSAPVLPCFNPTPEMHIPGQIRNMLEMIQVESMMEINNTDGANGMERLRVDISVQADLDQLLFNIPLDIQLDGPLRNTLVGNISRYYTHWSGSLEMTFMFCGSFMATGKLILCYTPPGGSCPTTRETAMLGTHIVWDFGLQSSITLIIPWISGSHYRMFNSDAKSTNANVGYVTCFMQTNLIVPSESSDTCSLIGFIAAKDDFSLRLMRDSPDIGQSNHLHGAEAAYQVESIIKTATDTVKSEINAELGVVPSLNAVETGATSNTEPEEAIQTRTVINQHGVSETLVENFLGRAALVSKKSFEYKNHASSSAGTHKNFFKWTINTKSFVQLRRKLELFTYLRFDAEITILTTVAVNGNNDSTYMGLPDLTLQAMFVPTGALTPKEQDSFHWQSGSNASVFFKISDPPARMTIPFMCINSAYSVFYDGFAGFEKNGLYGINPADTIGNLCVRIVNEHQPVGFTVTVRVYMKPKHIKAWAPRPPRTMPYMSIANANYKGRDTAPNTLNAIIGNRASVTTMPHNIVTTGPGFGGVFVGSFKIINYHLATIEERQSAIYVDWQSDVLVTPIAAHGRHQIARCKCNTGVYYCRHRDRSYPICFEGPGIQWIEQNEYYPARYQTNVLLAAGPAEAGDCGGLLVCPHGVIGLLTAGGGGIVAFTDIRNLLWLDTDVMEQGITDYIQNLGNAFGAGFTETISNKAKEVQDMLIGESSLLEKLLKALIKIISALVIVIRNSEDLITVTATLALLGCHDSPWSYLKQKVCSYLGIPYVPRQSESWLKKFTEACNALRGLDWLSQKIDKFINWLKTKILPEAREKYEFVQRLKQLPVIEKQVSTIEHSCPTTERQQALFNNVQYYSHYCRKYAPLYAVESKRVAALEKKINNYIQFKSKSRIEPVCLIIHGSPGTGKSVASNLIARAITEKLGGDIYSLPPDPKYFDGYKQQTVVLMDDLMQNPDGNDISMFCQMVSTVDFIPPMASLEEKGTLYTSPFLIATTNAGSIHAPTVSDSKALSRRFKFDVDIEVTDSYKDSNKLDMSRAVEMCKPDNCTPTNYKRCCPLICGKAIQFRDRRTNARSTVDMLVTDIIKEYRTRNSTQDKLEALFQGPPQFKEIKISVAPDTPAPDAINDLLRSVDSQEVRDYCQKKGWIVIHPSNELVVEKHISRAFITLQAIATFVSIAGVVYVIYKLFAGIQGPYTGIPNPKPKVPSLRTAKVQGPGFDFAQAIMKKNTVIARTEKGEFTMLGVYDRVAVIPTHASVGEIIYINDVETRVLDACALRDLTDTNLEITIVKLDRNQKFRDIRHFLPRCEDDYNDAVLSVHTSKFPNMYIPVGQVTNYGFLNLGGTPTHRILMYNFPTRAGQCGGVVTTTGKVIGIHVGGNGAQGFAAMLLHSYFTDTQGEIVSNEKSGMCINAPAKTKLQPSVFHQVFEGSKEPAVLNSKDPRLKTDFEEAIFSKYTGNKIMLMDEYMEEAVDHYVGCLEPLDISVDPIPLENAMYGMEGLEALDLTTSAGFPYLLQGKKKRDIFNRQTRDTSEMTKMLEKYGVDLPFVTFVKDELRSREKVEKGKSRLIEASSLNDSVAMRVAFGNLYATFHNNPGTATGSAVGCDPDIFWSKIPILLDGEIFAFDYTGYDASLSPVWFACLKKVLIKLGYTHQTSFIDYLCHSVHLYKDRKYVINGGMPSGSSGTSIFNTMINNIIIRTLLIKVYKGIDLDQFKMIAYGDDVIASYPHKIDPGLLAEAGKHYGLVMTPADKGTSFIDTNWENVTFLKRYFRADDQYPFLIHPVMPMKEIHESIRWTKDPRNTQDHVRSLCYLAWHNGEEAYNEFCRKIRSVPVGRALTLPAYSSLRRKWLDSF

SEQ ID NO: 168 - protein, 2194 aa, synthetic construct
  MOD_RES 1185: note = XXXXX
  MOD_RES 1957: note = XXXXX
MGAQVSRQQTGTHENANVATGGSSITYNQINFYKDSYAASASKQDFSQDPSKFTEPVAEALKAGAPVLKSPSAEACGYSDRVLQLKLGNSSIVTQEAANICCAYGEWPTYLPDNEAVAIDKPTQPETSTDRFYTLKSKKWESNSTGWWWKLPDALNQIGMFGQNVQYHYLYRSGFLCHVQCNATKFHQGTLLIVAIPEHQIGKKGTGTSASFAEVMKGAEGGVFEQPYLLDDGTSLACALVYPHQWINLRTNNSATIVLPWMNSAPMDFALRHNNWTLAVIPVCPLAGGTGNTNTYVPITISIAPMCAEYNGLRNAITQGVPTCLLPGSNQFLTTDDHSSAPAFPDFSPTPEMHIPGQVHSMLEIVQIESMMEINNVNDASGVERLRVQISAQSDMDQLLFNIPLDIQLEGPLRNTLLGNISRYYTHWSGSLEMTFMFCGSFMTTGKLIICYTPPGGSSPTDRMQAMLATHVVWDFGLQSSITIIIPWISGSHYRMFNTDAKAINANVGYVTCFMQTNLVAPVGAADQCYIVGMVAAKKDFNLRLMRDSPDIGQSAILPEQAATTQIGEIVKTVANTVESEIKAELGVIPSLNAVETGATSNTEPEEAIQTRTVINMHGTAECLVENFLGRSALVCMRSFEYKNHSTSTSSIQKNFFIWTLNTRELVQIRRKMELFTYLRFDTEITIVPTLRLFSSSNVSFSGLPNLTLQAMYVPTGARKPSSQDSFEWQSACNPSVFFKINDPPARLTIPFMSINSAYANFYDGFAGFEKKATVLYGINPANTMGNLCLRVVNSYQPVQYTLTVRVYMKPKHIKAWAPRAPRTMPYTNILNNNYAGRSAAPNAPTAIVSHRSTIKTMPNDINLTTAGPGYGGAFVGSYKIINYHLATDEEKERSVYVDWQSDVLVTTVAAHGKHQIARCRCNTGVYYCKHKNRSYPVCFEGPGIQWINESDYYPARYQTNTLLAMGPCQPGDCGGLLVCSHGVIGLVTAGGEGIVAFTDIRNLLWLEDDAMEQGITDYIQNLGSAFGTGFTETISEKAKEIQNMLVGEDSLLEKLLKALIKIVSAMVIVIRNSEDLVTVTATLALLGCNDSPWAFLKQKVCSYLGIPYTIRQSDSWLKKFTEACNALRGLDWLAQKIDKFINWLKTKILPEAREKHEFVQKLKQLPVIESQINTIEHSCPNSEXQQALFNNVQYYSHYCKKYAPLYALEAKRVSALERKINNYIQFKSKSRIEPVCLIIHGSPGTGKSVASNLIARAITEKLGGDSYSLPPDPKYFDGYKQQTVVLMDDLMQNPDGNDIAMFCQMVSTVDFIPPMASLEEKGTLYTSPFLIATTNAGSIHAPTVSDSKALARRFKFDMEIESMESYKDGVRLDMFKAVELCNPEKCRPTNYKKCCPLICGKAIQFRDKRTNVRYSVDMLVTEMIKEYRIRNSTQDKLEALFQGPPTFKEIKISVTPETPAPDAINDLLRSIDSQEVRDYCQKKGWIVMHPPTELVVDKHISRAFIALQAITTFVSIAGVVYVIYKLFAGIQGPYTGLPNQKPKVPTLRTAKVQGPSLDFAQAIMRKNTVIARTSKGEFTMLGIYDRIAVVPTHASVEEEIYINDVPVKVKDAYALRDINDVNLEITVVELDRNEKFRDIRGFLPKYEDDYNDAILSVNTSKFPNMYIPVGQTLNYGFLNLGGTPTHRILMYNFPTRAGQCGGVVTTTGKVIGIHVGGNGAQGFAAMLLQNYFTEKQGEIVSIEKTGVFINAPAKTKLEPSVFHEVFEGVKEPAVLHSKDKRLKVDFEEAIFSKYVGNKTMLMDEYMEEAVDHYVGCLEPLDISTEPIKLEEAMYGMDGLEALDLTTSAGYPYLLQGKKKRDIFNRQTRDTTEMTKMLDKYGVDLPFVTFVKDELRSREKVEKGKSRLIEASSLNDSVAMRVAFGNLYATFHKNPGVATGSAVGCDPDLFWSKIPVXLDGKIFAFDYTGYDASLSPVWFACLKKTLVKLGYTHQTAFVDYLCHSVHLYKDRKYIVNGGMPSGSSGTSIFNTMINNIIIRTLLLKVYKGIDLDQFKMIAYGDDVIASYPHEIDPGLLAKAGKEYGLIMTPADKSSGFTETTWENVTFLKRYFRADEQYPFLIHPVMPMKEIHESIRWTKDPRNTQDHVRSLCLLAWHNGEETYNEFCRKIRTVPVGRALALPVYSSLRRKWLDSF

SEQ ID NO: 169 - protein, 2193 aa, synthetic construct
MGSQVSTQRSGSHENSNSATEGSTINYTTINYYKDSYAATAGKQSLKQDPDKFANPVKDIFTEMAAPLKSPSAEACGYSDRVAQLTIGNSTITTQEAANIIVGYGEWPSYCSDSDATAVDKPTRPDVSVNRFYTLDTKLWEKSSKGWYWKFPDVLTETGVFGQNAQFHYLYRSGFCIHVQCNASKFHQGALLVAVLPEYVIGTVAGGTGTEDSHPPYKQTQPGADGFELQHPYVLDAGIPISQLTVCPHQWINLRTNNCATIIVPYINALPFDSALNHCNFGLLVVPISPLDYDQGATPVIPITITLAPMCSEFAGLRQAVTQGFPTELKPGTNQFLTTDDGVSAPILPNFHPTPCIHIPGEVRNLLELCQVETILEVNNVPTNATSLMERLRFPVSAQAGKGELCAVFRADPGRNGPWQSTLLGQLCGYYTQWSGSLEVTFMFTGSFMATGKMLIAYTPPGGPLPKDRATAMLGTHVIWDFGLQSSVTLVIPWISNTHYRAHARDGVFDYYTTGLVSIWYQTNYVVPIGAPNTAYIIALAAAQKNFTMKLCKDASDILQTGTIQGDRVADVIESSIGDSVSRALTQALPAPTGQNTQVSSHRLDTGKVPALQAAEIGASSNASDESMIETRCVLNSHSTAETTLDSFFSRAGLVGEIDLPLEGTTNPNGYANWDIDITGYAQMRRKVELFTYMRFDAEFTFVACTPTGEVVPQLLQYMFVPPGAPKPDSRESLAWQTATNPSVFVKLSDPPAQVSVPFMSPASAYQWFYDGYPTFGEHKQEKDLEYGACPNNMMGTFSVRTVGTSKSKYPLVVRIYMRMKHVRAWIPRPMRNQNYLFKANPNYAGNSIKPTGTSRTAITTLGKFGQQSGAIYVGNFRVVNRHLATHNDWANLVWEDSSRDLLVSSTTAQGCDTIARCNCQTGVYYCNSRRKHYPVSFSKPSLIYVEASEYYPARYQSHLMLAQGHSEPGDCGGILRCQHGVVGIVSTGGNGLVGFADVRDLLWLDEEAMEQGVSDYIKGLGDAFGTGFTDAVSREVEALKSYLIGSEGAVEKILKNLIKLISALVIVIRSDYDMVTLTATLALIGCHGSPWAWIKAKTASILGIPIAQKQSASWLKKFNDMANAAKGLEWVSNKISKFIDWLKEKIVPAAKEKVEFLNNLKQLPLLENQISNLEQSAASQEDLEVMFGNVSYLAHFCRKFQPLYATEAKRVYALEKRMNNYMQFKSKHRIEPVCLIIRGSPGTGKSLATGIIARAIADKYHSSVYSLPPDPDHFDGYKQQVVTVMDDLCQNPDGKDMSLFCQMVSTVDFIPPMASLEEKGVSFTSKFVIASTNATNIIVPTVSDSDAIRRRFYMDCDIEVTDSYKTDLGRLDAGRAAKLCSENNTANFKRCSPLVCGKAIQLRDRKSKVRYSVDTVVSELIREYSNRSAIGNTIEALFQGPPKFRPIRIGLEEKPAPDAISDLLASVDSEEVRQYCRDQGWIIPETPTNVERHLNRAVLVMQSIATVVAVVSLVYVIYKLFAGFQGAYSGAPKQVLKKPALRTATVQGPSLDFALSLLRRNVRQVQTDQGHFTMLGVRDRLAVLPRHSQPGKTIWIEHKLVNVLDAVELVDEQGVNLELTLITLDTNEKFRDITKFIPENISTASDATLVINTEHMPSMFVPVGDVVQYGFLNLSGKPTHRTMMYNFPTKAGQCGGVVTSVGKVIGIHIGGNGRQGFCAGLKRSYFASEQGEIQWVKPNKETGRLNINGPTRTKLEPSVFHDVFEGSKEPAVLHSKDPRLEVDFEQALFSKYVGNTLHVPDEYIREAALHYANQLKQLDIDTTQMSMEEACYGTDNLEAIDLHTSAGYPYSALGIKKRDILDPTTRDVSKMKFYMDKYGLDLPYSTYVKDELRSIDKIKKGKSRLIEASSLNDSVYLRMAFGHLYETFHANPGTVTGSAVGCNPDVFWSKLPILLPGSLFAFDYSGYDASLSPVWFRALELVLREIGYSEEAVSLIEGINHTHHVYRNKTYCVLGGMPSGCSGTSIFNTMINNIIIRALLIKTFKGIDLDELNMVAYGDDVLASYPFPIDCLELAKTGKEYGLTMTPADKSPCFNEVNWENATFLKRGFLPDEQFPFLIHPTMPMKEIHESIRWTKDARNTQDHVRSLCLLAWHNGKQEYEKFVSSIRSVPIGKALAIPNYENLRRNWLELF

SEQ ID NO: 170 - protein, 2193 aa, synthetic construct
MGSQVSTQRSGSHENSNSASEGSTINYTTINYYKDAYAASAGRQDMSQDPKKFTDPVMDVIHEMAPPLKSPSAEACGYSDRVAQLTIGNSTITTQEAANIIIAYGEWPEYCKDADATAVDKPTRPDVSVNRFFTLDTKSWAKDSKGWYWKFPDVLTEVGVFGQNAQFHYLYRSGFCVHVQCNASKFHQGALLVAILPEYVLGTIAGGDGNENSHPPYVTTQPGQVGAVLTNPYVLDAGVPLSQLTVCPHQWINLRTNNCATIIVPYMNTVPFDSALNHCNFGLIVVPVVPLDFNAGATSEIPITVTIAPMCAEFAGLRQAIKQGIPTELKPGTNQFLTTDDGVSAPILPGFHPTPAIHIPGEVRNLLEICRVETILEVNNLQSNETTPMQRLCFPVSVQSKTGELCAVFRADPGRNGPWQSTILGQLCRYYTQWSGSLEVTFMFAGSFMATGKMLIAYTPPGGGVPADRLTAMLGTHVIWDFGLQSSVTLVIPWISNTHYRAHAKDGYFDYYTTGTITIWYQTNYVVPIGAPTTAYIVALAAAQDNFTMKLCKDTEDIEQSANIQGDGIADMIDQAVTSRVGRALTSLQVEPTAANTNASEHRLGTGLVPALQAAETGASSNAQDENLIETRCVLNHHSTQETTIGNFFSRAGLVSIITMPTTGTQNTDGYVNWDIDLMGYAQMRRKCELFTYMRFDAEFTFVAAKPNGELVPQLLQYMYVPPGAPKPTSRDSFAWQTATNPSIFVKLTDPPAQVSVPFMSPASAYQWFYDGYPTFGAHPQSNDADYGQCPNNMMGTFSIRTVGTEKSPHSITLRVYMRIKHVRAWIPRPLRNQPYLFKTNPNYKGNDIKCTSTSRDKITTLGKFGQQSGAIYVGNYRVVNRHLATHNDWANLVWEDSSRDLLVSSTTAQGCDTIARCDCQTGVYYCSSRRKHYPVSFSKPSLIFVEASEYYPARYQSHLMLAVGHSEPGDCGGILRCQHGVVGIVSTGGNGLVGFADVRDLLWLDEEAMEQGVSDYIKGLGDAFGTGFTDAVSREVEALKNHLIGSEGAVEKILKNLIKLISALVIVIRSDYDMVTLTATLALIGCHGSPWAWIKAKTASILGIPIAQKQSASWLKKFNDMANAAKGLEWISNKISKFIDWLKEKIIPAAKEKVEFLNNLKQLPLLENQISNLEQSAASQEDLEAMFGNVSYLAHFCRKFQPLYATEAKRVYALEKRMNNYMQFKSKHRIEPVCLIIRGSPGTGKSLATGIIARAIADKYHSSVYSLPPDPDHFDGYKQQVVTVMDDLCQNPDGKDMSLFCQMVSTVDFIPPMASLEEKGVSFTSKFVIASTNASNIIVPTVSDSDAIRRRFYMDCDIEVTDSYKTDLGRLDAGRAARLCSENNTANFKRCSPLVCGKAIQLRDRKSKVRYSVDTVVSELIREYNNRYAIGNTIEALFQGPPKFRPIRISLEEKPAPDAISDLLASVDSEEVRQYCRDQGWIIPETPTNVERHLNRAVLIMQSIATVVAVVSLVYVIYKLFAGFQGAYSGAPKQTLKKPILRTATVQGPSLDFALSLLRRNIRQVQTDQGHFTMLGVRDRLAVLPRHSQPGKTIWVEHKLINILDAVELVDEQGVNLELTLVTLDTNEKFRDITKFIPENISAASDATLVINTEHMPSMFVPVGDVVQYGFLNLSGKPTHRTMMYNFPTKAGQCGGVVTSVGKVIGIHIGGNGRQGFCAGLKRSYFASEQGEIQWVKPNKETGRLNINGPTRTKLEPSVFHDVFEGNKEPAVLHSRDPRLEVDFEQALFSKYVGNTLHEPDEYIKEAALHYANQLKQLDINTSQMSMEEACYGTENLEAIDLHTSAGYPYSALGIKKRDILDPTTRDVSKMKFYMDKYGLDLPYSTYVKDELRSIDKIKKGKSRLIEASSLNDSVYLRMAFGHLYETFHANPGTITGSAVGCNPDTFWSKLPILLPGSLFAFDYSGYDASLSPVWFRALELVLREVGYSEEAVSLIEGINHTHHVYRNKTYCVLGGMPSGCSGTSIFNSMINNIIIRTLLIKTFKGIDLDELNMVAYGDDVLASYPFPIDCLELARTGKEYGLTMTPADKSPCFNEVNWGNATFLKRGFLPDEQFPFLIHPTMPMKEIHESIRWTKDARNTQDHVRSLCLLAWHNGKQEYEKFVSTIRSVPVGKALAIPNYENLRRNWLELF

SEQ ID NO: 171 - protein, 2193 aa, synthetic construct
MGSQVSTQRSGSHENSNSATEGSTINYTTINYYKDSYAATAGKQSLKQDPDKFANPVKDIFTEMAAPLKSPSAEACGYSDRVAQLTIGNSTITTQEAANIIVGYGEWPSYCSDDDATAVDKPTRPDVSVNRFYTLDTKLWEKSSKGWYWKFPDVLTETGVFGQNAQFHYLYRSGFCIHVQCNASKFHQGALLVAILPEYVIGTVAGGTGTEDSHPPYKQTQPGADGFELQHPYVLDAGIPISQLTVCPHQWINLRTNNCATIIVPYMNTLPFDSALNHCNFGLLVVPISPLDFDQGATPVIPITITLAPMCSEFGGLRQAVTQGFPTELKPGTNQFLTTDDGVSAPILPNFHPTPCIHIPGEVRNLLELCQVETILEVNNVPTNATSLMERLRFPVSAQAGKGELCAVFRADPGRDGPWQSTMLGQLCGYYTQWSGSLEVTFMFTGSFMATGKMLIAYTPPGGPLPKDRATAMLGTHVIWDFGLQSSVTLVIPWISNTHYRAHARDGVFDYYTTGLVSIWYQTNYVVPIGAPNTAYILALAAAQKNFTMKLCKDTSHILQTASIQGDRVADVIESSIGDSVSRALTQALPAPTGQNTQVSSHRLDTGEVPALQAAEIGASSNTSDESMIETRCVLNSHSTAETTLDSFFSRAGLVGEIDLPLEGTTNPNGYANWDIDITGYAQMRRKVELFTYMRFDAEFTFVACTPTGEVVPQLLQYMFVPPGAPKPESRESLAWQTATNPSVFVKLTDPPAQVSVPFMSPASAYQWFYDGYPTFGEHKQEKDLEYGACPNNMMGTFSVRTVGSSKSKYPLVVRIYMRMKHVRAWIPRPMRNQNYLFKANPNYAGNSIKPTGTSRNAITTLGKFGQQSGAIYVGNFRVVNRHLATHNDWANLVWEDSSRDLLVSSTTAQGCDTIARCNCQTGVYYCNSKRKHYPVSFSKPSLIYVEASEYYPARYQSHLMLAAGHSESGDCGGILRCQHGVVGIASTGGNGLVGFADVRDLLWLDEEAMEQGVSDYIKGLGDAFGTGFTDAVSREVEALRNHLIGSDGAVEKILKNLIKLISALVIVIRSDYDMVTLTATLALIGCHGSPWAWIKAKTASILGIPIAQKQSASWLKKFNDMASAAKGLEWISNKISKFIDWLREKIVPAAKEKAEFLTNLKQFPLLENQITHLEQSAASQEDLEAMFGNVSYLAHFCRKFQPLYATEAKRVYVLEKRMNNYMQFKSTHRIEPVCLIIRGSPGTGKSLATGIIARAIADKYHSSVYSLPPDPDHFDGYKQQVVTVMDDLCQNPDGKDMSLFYQMVSTVDIIPPMASLEEKGVSFTSKFVIASTNASNIIVPTVSDSDAIRRRFYMDCDIEVTDSSKTDLGRLDAGRAAKLCSENNTANFKRCSPLVCGKAIQLRDRKSKVRYSVDTVVSELIREYNSRSAIGNTIEALFQGPPKFRPIRISLEEKPAPDAISDLLASVDSEEVRQYCREQGWIIPETPTNVERHLNRAVLVMQSIATVVAVVSLVYVIYKLFAGFQGAYSGAPNQVLKKPVLRTATVQGPSLDFALSLLRRNIRQVQTDQGHFTMLGVRDRLAVLPRHSQPGKTIWVEHKLVNILDAAELVDEQGVNLELTLVTLDTNEKFRDITKFIPETISGASDATLVINTEHMPSMFVPVGDVVQYGFLNLSGKPTHRTMMYNFPTKAGQCGGVVTSVGKIIGIHIGGNGRQGFCAGLKRSYFASEQGEIQWVKSNKETGRLNINGPTRTKLEPSVFHDVFEANKEPAVLTSKDPRLEVDFEQALFSKYVGNVLHEPDEYVHQAALHYANQLKQLDINTKKMSMEEACYGTDNLEAIDLHTSAGYPYSALGIKKRDILDPATRDVSKMKSYMDKYGLDLPYSTYVKDELRSLDKIKKGKSRLIEASSLNDSVYLRMTFGHLYEVFHANPGTVTGSAVGCNPDVFWSKLPILLPGSLFAFDYSGYDASLSPVWFRALEVVLREIGYSEEAVSLIEGINHTHHIYRNKTYCVLGGMPSGCSGTSIFNSMINNIIIRTLLIKTFKGIDLDELNMVAYGDDVLASYPFPIDCLELAKTGKEYGLTMTPAGKSPCFNEVTWENATFLKRGFLPDHQFPFLIHPTMPMKEIHESIRWTKDARNTQDHVRSLCLLAWHNGKDEYEKFVSTIRSVPVGKALAIPNFENLRRNWLELF

SEQ ID NO: 172 - protein, 2206 aa, synthetic construct
MGAQVSTQKTGAHENQNVAANGSTINYTTINYYKDSASNSATRQDLSQDPSKFTEPVKDLMLKTAPALNSPNVEACGYSDRVRQITLGNSTITTQEAANAIVAYGEWPTYINDSEANPVDAPTEPDVSSNRFYTLESVSWKTTSRGWWWKLPDCLKDMGMFGQNMYYHYLGRSGYTIHVQCNASKFHQGALGVFLIPEFVMACNTESKTSYVSYINANPGERGGEFTNTYNPSNTDVSEGRQFAALDYLLGSGVLAGNAFVYPHQIINLRTNNSATIVVPYVNSLVIDCMAKHNNWGIVILPLAPLAFAATSSPQVPITVTIAPMCTEFNGLRNITIPVHQGLPTMNTPGSNQFLTSDDFQSPCALPNFDVTPPIHIPGEVKNMMELAEIDTLIPMNAVDGKVNTMEMYQIPLNDNLSKAPIFCLSLSPASDKRLSHTMLGEILNYYTHWTGSIRFTFLFCGSMMATGKLLLSYSPPGAKPPTNRKDAMLGTHIIWDLGLQSSCSMVAPWISNTVYRRCARDDFTEGGFITCFYQTRIVVPASTPTSMFMLGFVSACPDFSVRLLRDTSHISQSKLIARTQGIEDLIDTAIKNALRVSQPLRPSQLKQPNGVNSQEVPALTAVETGASGQAIPSDVVETRHVINYKTRSESCLESFFGRAACVTILSLTNSSKSGEEKKHFNIWNITYTDTVQLRRKLEFFTYSRFDLEMTFVFTENYPSTASGEVRNQCDQIMYIPPGAPRPSSWDDYTWQSSSNPSIFYMYGNAPPRMSIPYVGIANAYSHFYDGFARVPLEGENTDAGDTFYGLVSINDFGVLAVRAVNRSNPHTIHTSVRVYMKPKHIRCWCPRPPRAVLYRGEGVDMISSAIQPLTKVDSITTFGFGHQNKAVYVAGYKICNYHLATPSDHLNAISVLWDRDLMVVESRAQGTDTIARCSCRCGVYYCESRRKYYLVTFTGPTFRFMEANDYYPARYQSHMLIGCGFAEPGDCGGILRCTHGVIGIITAGGEGIVAFADIRDLWVYEEEAMEQGITSYIESLGTAFGAGFTHTISEKVTELTTMVTSTITEKLLKNLVKIVSALVIVVRNYEDTTTILATLALLGCDISPWQWLKKKACDLLEIPHVMRQGDGWMKKFTEACNAAKGLRWVSNKISKFVDWLKCKIIPEAKDKVEFLTKLKQLDMLENQIATIHQSCPSQEQQEILFNNVRWLAVQSRRFAPLYAVEARRISKMESTINNYIQFKSKHRIEPVCMLVHGSPGTGKGIASSLIGRAIAERETTSVYSVPLAPSHFDGYKQQGYDMDDLNQNPDGMDMKLFCQMVSTVEFIPPMASLEEKGILFTSDYVLASTNSHSIAPPTVAHSDALTRRFAFDVEVYTMSEHSVKGKLNMATATQLCKDCPTPANFKKCCPLVCGKALQLMDRYTRQRFTVDEITTLIMNEKNRRANIGNCMEALFQGPLRYKDLKIDVKTVPPPECISDLLQAVDSQEVRDYCEKKGWIVNVTSQIQLERNINRAMTILQAVTTFAAVAGVVYVMYKLFAGQQGAYTGLPNKKPNVPTIRIAKVQGPGFDYAVAMAKRNIVTATTTKGEFTMLGVHDNVAILPTHAAPGETIIVDGKEVEILDARALEDQAGTNLEITIITLKRNEKFRDIRPHIPTQITETNDGVLIVNTSKYPNMYVPVGAVTEQGYLNLSGRQTARTLMYNFPTRAGQCGGIITCTGKVIGMHVGGNGSHGFAAALKRSYFTQNQGEIQWMRSSKEVGYPIINAPSKTKLEPSAFHYVFEGVKEPAVLTKNDPRLKTDFEEAIFSKYVGNKITEVDEYMKEAVDHYAGQLMSLDINTEQMCLEDAMYGTDGLEALDLSTSAGYPYVAMGKKKRDILDKQTRDTKEMQRLLDTYGINLPLVTYVKDELRSKTKVEQGKSRLIEASSLNDSVAMRMAFGNLYAAFHKNPGVVTGSAVGCDPDLFWSKIPVLMEEKLFAFDYTGYDASLSPAWFEALKMVLEKIGFGNRVDYIDYLNHSHHLYKNKTYCVKGGMPSGCSGTSIFNSMINNLIIRTLLLRTYKGIDLDHLKMIAYGDDVIASYPHEVDASLLAQSGKDYGLTMTPADKSATFETVTWENVTFLKRFFRADEKYPFLVHPVMPMKEIHESIRWTKDPRNTQDHVRSLCLLAWHNGEEEYNKFLAKIRSVPIGRALLLPEYSTLYRRWLDSF

SEQ ID NO: 173 - protein, 2214 aa, synthetic construct
MGAQVSSQKVGAHENTNVATGGSTVNYTTINYYKDSASNAASKLDFSQDPSKFTEPVKDIMLKSAPALNSPNVEACGYSDRVRQITLGNSTITTQEAANAVVAYGEWPSYLDDREANPIDAPTEPDVSSNRFYTLDSVQWTSTSRGWWWKLPDALKDMGMFGQNMYYHYLGRSGYTVHVQCNASKFHQGALGVFAIPEYVMACNTEAKTSYVSYINANPGEKGGVFSSTYNPSEEASEGRKFAALDYLLGCGVLAGNAFVYPHQIINLRTNNSATLVLPYVNSLAIDCMAKHNNWGLVILPLCKLDYAPNSSTEIPITVTIAPMFTEFNGLRNITVPATQGLPTMLTPGSSQFLTSDDFQSPCALPNFDVTPPIHIPGEVFNMMELAEIDSMIPMNSVTGKANTMEMYPIPLDDKGGKTPIFSISLSPASDKRLQYTMLGEILNYYTHWTGSLRFTFLFCGSMMATGKILLSYSPPGAKPPTTRKDAMLGTHIIWDLGLQSSCTMLAPWISNTVYRRCIKDDFTEGGYITCFYQTRIVVPSGTPTSMFMLAFVSACPDFSVRLLRDTNHISQRTLFARAQGIEETIDTVISNALQLSQPKPQKQLTAQSTPSTSGVNSQEVPALTAVETGVSGQAIPSDVIETRHVVNYKTRSESTLESFFGRSACVTMLEVENFNATTEADKKKQFTTWAITYTDTVQLRRKLEFFTYSRFDLEMTFVITERYYTSNTGYARNQVYQLMYIPPGAPRPTAWDDYTWQSSSNPSVFYTYGSAPPRISIPYVGIANAYSHFYDGFARVPLKDETVDSGDTYYGLVTINDFGTLAVRVVNEFNPARIISKIRVYMKPKHVRCWCPRPPRAVPYRGEGVDFKQDSITPLIAVENINTFGGFGHQNMAVYVAGYKICNYHLATPEDHDNAVRVLWNRDLMIVSSRAQGSDTIARCNCRTGVYYCKSMKKYYPVTVTEPTFQYMEANDYYPARYQTHMLLGMGFAEPGDCGGILRCNHGVMGIVTAGGNGIVAFADIRDLWVYEEEAMEQGITSYIESLGAAFGSGFTNQIGEKVSELTSMVTSSITEKLLKTLIKIISTLVIISRNYEDTTTVLATLALLGCDYSPWQWIKKKACDVLELPYVMRQGDSWLKKFTEACNAAKGLEWISNKISKFIDWLKERIIPEAKDKVEFITKLKQLGILENQINTIHQSCPSQEQQEILFNNVRWLAIQSRRFAPLYAVEAKRISKLENTINNYIQFKSKHRIEPVCMLIHGSPGTGKSIATSLIGRAIAEKENTSTYSLPPDPTHFDGYKQQGVVIMDDLNQNPDGNDMKLFCQMVSTVEFIPPMASLEEKGILFTSDYVLASTNSHTITPPTVSHSDALNRRFAFDMEVYTMSEHSIKGKLNMATATQLCKDCPQPANFKKCCPLVCGKALQLMDKNTRQRFTLDEITTLVINERNRRANIGNCMEALFQGPIQYRDVMIDIKETPAPDYINDLLQSVDSQELRDYCEKKGWIARLNNDLVMERNLNRAMTILQAVTTFAAVAAVVYVMYKLFAGHQGAYTGLPNKKPSVPTVRTAKVQGPGFDYAVAMAKRNILTATTSKGEFTMLGVHDNVAILPTHAAPGDSIVIDGKEVEVLDAEALEDQSGTNLEITIVKLKRNEKFRDIRPHIPTQITETNDGVLIVNTSKYPNMYVPVGAVTEQGYLNLGGRQTARTLMYNFPTRAGQCGGVITCTGKVIGMHVGGNGSHGFAAALKRSYFTQSQGEIQWMRPSKEVGYPVINAPSKTKLEPSVFHHVFEGAKEPAVLTKNDPRLKTDFEEAIFSKYVGNKITEVDEYMKEAVDHYAGQLMSLDINTEQMCLEDAMYGTDGLEALDLSTSAGYPYVAMGKKKRDILNKQTRDTKEMQRLLDTYGINLPLVTYVKDELRSKTKVEQGKSRLIEASSLNDSVAMRMAFGNLYAAFHKNPGVVTGSAVGCDPDLFWSKIPVLMEEKLFAFDYTGYDASLSPAWFEALKMVLEKIGFGDRVDYIDYLNHSHHLYKNKTYCVKGGMPSGCSGTSIFNSMINNLIIRTLLLKTYKGIDLDHLKMIAYGDDVIASYPHEVDASLLAQSGKDYGLTMTPADKSATFETVTWENVTFLKRFFRADEKYPFLVHPVMPMKEIHESIRWTKDPRNTQDHVRSLCLLAWHNGEEEYNKFLAKVRSVPIGRALLLPEYSTLYRRWLDSF

SEQ ID NO: 174 - protein, 2209 aa, synthetic construct
MGAQVSSQKVGAHENSNRAYGGSTINYTTINYYRDSASNAASKQDFSQDPSKFTEPIKDVLIKTAPMLNSPNIEACGYSDRVLQLTLGNSTITTQEAANSVVAYGRWPEYLRDSEANPVDQPTEPDVAACRFYTLDTVSWTKESRGWWWKLPDALRDMGLFGQNMYYHYLGRSGYTVHVQCNASKFHQGALGVFAVPEMCLAGDSNTTTMHTSYQNANPGEKGGTFTGTFTPDNNQTSPARRFCPVDYLLGNGTLLGNAFVFPHQIINLRTNNCATLVLPYVNSLSIDSMVKHNNWGIAILPLAPLNFASESSPEIPITLTIAPMCCEFNGLRNITLPRLQGLPVMNTPGSNQYLTADNFQSPCALPEFDVTPPIDIPGEVKNMMELAEIDTMIPFDLSATKKNTMEMYRVRLSDKPHTDDPILCLSLSPASDPRLSHTMLGEILNYYTHWAGSLKFTFLFCGFMMATGKLLVSYAPPGADPPKKRKEAMLGTHVIWDIGLQSSCTMVVPWISNTTYRQTIDDSFTEGGYISVFYQTRIVVPLSTPREMDILGFVSACNDFSVRLLRDTTHIEQKALAQGLGQMLESMIDNTVRETVGAATSRDALPNTEASGPTHSKEIPALTAVETGATNPLVPSDTVQTRHVVQHRSRSESSIESFFARGACVTIMTVDNPASTTNKDKLFAVWKITYKDTVQLRRKLEFFTYSRFDMELTFVVTANFTETNNGHALNQVYQIMYVPPGAPVPEKWDDYTWQTSSNPSIFYTYGTAPARISVPYVGISNAYSHFYDGFSKVPLKDQSAALGDSLYGAASLNDFGILAVRVVNDHNPTKVTSKIRVYLKPKHIRVWCPRPPRAVAYYGPGVDYKDGTLTPLSTKDLTTYGFGHQNKAVYTAGYKICNYHLATQDDLQNAVNVMWSRDLLVTESRAQGTDSIARCNCNAGVYYCESRRKYYPVSFVGPTFQYMEANNYYPARYQSHMLIGHGFASPGDCGGILRCHHGVIGIITAGGEGLVAFSDIRDLYAYEEEAMEQGITNYIESLGAAFGSGFTQQISDKITELTNMVTSTITEKLLKNLIKIISSLVIITRNYEDTTTVLATLALLGCDASPWQWLRKKACDVLEIPYVIKQGDSWLKKFTEACNAAKGLEWVSNKISKFIDWLKEKIIPQARDKLEFVTKLRQLEMLENQISTIHQSCPSQEHQEILFNNVRWLSIQSKRFAPLYAVEAKRIQKLEHTINNYIQFKSKHRIEPVCLLVHGSPGTGKSVATNLIARAIAERENTSTYSLPPDPSHFDGYKQQGVVIMDDLNQNPDGADMKLFCQMVSTVEFIPPMASLEEKGILFTSNYVLASTNSSRISPPTVAHSDALARRFAFDMDIQVMNEYSRDGKLNMAMATEMCKNCHQPANFKRCCPLVCGKAIQLMDKSSRVRYSIDQITTMIINERNRRSNIGNCMEALFQGPLQYKDLKIDIKTSPPPECINDLLQAVDSQEVRDYCEKKGWIVNITSQVQTERNINRAMTILQAVTTFAAVAGVVYVMYKLFAGHQGAYTGLPNKKPNVPTIRTAKVQGPGFDYAVAMAKRNIVTATTSKGEFTMLGVHDNVAILPTHASPGESIVIDGKEVEILDAKALEDQAGTNLEITIITLKRNEKFRDIRPHIPTQITETNDGVLIVNTSKYPNMYVPVGAVTEQGYLNLGGRQTARTLMYNFPTRAGQCGGVITCTGKVIGMHVGGNGSHGFAAALKRSYFTQSQGEIQWMRPSKEVGYPIINAPSKTKLEPSAFHYVFEGVKEPAVLTKNDPRLKTDFEEAIFSKYVGNKITEVDEYMKEAVDHYAGQLMSLDINTEQMCLEDAMYGTDGLEALDLSTSAGYPYVAMGKKKRDILNKQTRDTKEMQKLLDTYGINLPLVTYVKDELRSKTKVEQGKSRLIEASSLNDSVAMRMAFGNLYAAFHKNPGVITGSAVGCDPDLFWSKIPVLMEEKLFAFDYTGYDASLSPAWFEALKMVLEKIGFGDRVDYIDYLNHSHHLYKNKTYCVKGGMPSGCSGTSIFNSMINNLIIRTLLLKTYKGIDLDHLKMIAYGDDVIASYPHEVDASLLAQSGKDYGLTMTPADKSATFETVTWENVTFLKRFFRADEKYPFLIHPVMPMKEIHESIRWTKDPRNTQDHVRSLCLLAWHNGEEEYNKFLAKIRSVPIGRALLLPEYSTLYRRWLDSF

SEQ ID NO: 175 - protein, 2207 aa, synthetic construct
MGAQVSSQKVGAHENSNRAYGGSTINYTTINYYRDSASNAANKQDFAQDPSKFTEPIKDVLIKTAPMLNSPNIEACGYSDRVMQLTLGNSTITTQEAANSVVAYGRWPEYIRDAEANPVDQPTEPDVAACRFYTLDTVTWNKESRGWWWKLPDALKDMGLFGQNMFYHYLGRAGYTVHVQCNASKFHQGALGVFAVPEMCLAGDSTIHMFTKYENANPGEKGGVFKGSFTPDNNVTSPAREFCPVDYLFGSGVLAGNAFVYPHQIINLRTNNCATLVLPYVNSLSIDSMTKHNNWGIAILPLAPLDFATESSTEIPITLTIAPMCCEFNGLRNITVPRTQGLPVLNTPGSNQYLTADNYQSPCAIPEFDVTPPIDIPGEVHNMMELAEIDTMIPLNLTNQRKNTMDMYRVELNDAAHTDSPILCLSLSPASDPRLSHTMLGEILNYYTHWAGSLKFTFLFCGSMMATGKLLVSYAPPGAEAPKSRKEAMLGTHVIWDIGLQSSCTMVVPWISNTTYRQTINDSFTEGGYISMFYQTRVVVPLSTPRKMDILGFVSACNDFSVRLLRDTTHITQVAMPQGIGDVIEGAIEGITKNALVPLATTSSLPDTKPSGPAHSKEIPALTAVETGATNPLVPSDTVQTRHVIQRRTRSESTVESFFARGACVAIIEVDNDAPTKRASRLFSVWKITYKDTVQLRRKLEFFTYSRFDMEFTFVVTSNYTESNNGHALNQVYQIMYIPPGAPIPGKWDDYTWQTSSNPSVFYTYGAPPARISVPYVGIANAYSHFYDGFAKVPLANQTSAEGDSLYGAASLNDFGSLAVRVVNDHNPTRLTSKIRVYMKPKHVRVWCPRPPRAVPYYGPGVDYKDGLTPLPEKELTTYGFGHQNKAVYTAGYKICNYHLATQEDLQNAVSVMWDRDLLVVESRAPGTDSIARCSCNAGVYYCESRRKYYPVSFIGPTFQFMEANDYYPARYQSHMLIGHGFASPGDCGGILRCQHGVIGIITAGGEGLVAFSDIRDLHAYEEEAMEQGITNYIESLGAAFGSGFTQQIGDKVSELANMVTSTITEKLLKNLIKIISSLVIITRNYEDTTTVLATLALLGCDISPWQWLKKKACDILEIPYAIKQGDGWLKKFTEACNAAKGLEWVSSKISKFISWLQDKIIPQARDKLEFVTKLKQLEMLENQISTIHQSCPSQEHQEILFNNVRWLSIQSKRFAPLYAHEAKRIQKLEHTINNYIQFKSKHRIEPVCLLVHGSPGTGKSVATNLIARAIAEKENTSTYSLPPDPSHFDGYKQQGVVIMDDLNQNPDGADMKLFCQMVSTVEFIPPMASLEEKGILFTSNYVLASTNSSRITPPTVAHSDALARRFAFDMDIQVMSEYSRDGKLNMAMATEMCKNCHQPANFKRCCPLVCGKAIQLMDKSSRVRYSVDQITTMIINERNRRSNIGNCMEALFQGPLQYRDLKIDVKTSPPPECINDLLQAVDSQEVRDYCEKKGWIVNITSQVQTERNINRAMTILQAVTTFAAVAGVVYVMYKLFAGHQGAYTGLPNKRPNVPTIRTAKVQGPGFDYAVAMAKRNIVTATTSKGEFTMLGVHDNVAILPTHATSGESIVIDGKEVEILDAKALEDQAGTNLEITIVTLKRNEKFRDIRPHIPTQITETNDGVLIVNTSKYPNMYVPVGAVTEQGYLNLGGRQTARTLMYNFPTRAGQCGGIITCTGKVIGMHVGGNGSHGFAAALKRSYFTQVQGEIQWMKPSKEVGYPIINAPSKTKLEPSAFHYVFEGVKEPAVLTKNDPRLKTDFEEAIFSKYVGNKITEVDEYMKEAVDHYAGQLMSLDINTEQMCLEDAMYGTDGLEALDLSTSAGYPYVAMGKKKRDILNKQTRDTKEMQRLLDTYGINLPLVTYVKDELRSKTKVEQGKSRLIEASSLNDSVAMRMAFGNLYASFHKNPGVVTGSAVGCDPDLFWSKIPVLMEEKLFAFDYTGYDASLSPAWFEALKMVLEKIGFGDRVDYIDYLNHSHHLYKNKTYCVKGGMPSGCSGTSIFNSMINNLIIRTLLLKTYKGIDLDHLKMIAYGDDVIASYPHEVDASLLAQSGKDYGLTMTPADKSATFETVTWENVTFLKRFFRADEKYPFLVHPVMPMKEIHESIRWTKDPRNTQDHVRSLCLLAWHNGEEEYNKFLAKIRSVPIGRALLLPEYSTLYRRWLDSF

SEQ ID NO: 176 - protein, 2191 aa, synthetic construct
MGAQVSTQKTGAHETGLSASGNSIIHYTNINYYKDAASNSANRQDFTQDPGKFTEPVKDIMAKTLPALNSPSAEECGYSDRVRSITLGNSTITTQESANVVVGYGVWPDYLKDDEATAEDQPTNPDVATCRFYTLDSVSWMKESQGWWWKFPDALRDMGLFGQNMQYHYLGRSGYTIHVQCNASKFHQGCLLVVCVPEAEMGAATVNEKINREHLSNGEVANTFTGTKSSNTNGVQQAVFNAGMGVRVGNLTVFPHQWINLRTNNCATIVMPYINSVPMDNMFRHYNFTLMIIPFAKLDYAAGSSTYIPITVTVAPMCAEYNGLRLAGHQGLPVMSTPGSNQFLTSDDYQSPTAMPQFDVTPEMHIPGEVKNLMEIAEVDSVVPVNNVNENVNSLEAYRIPVHSVTETGAQVFGFTLQPGADSVMERTLHGEILNYYANWSGSIKLTFMYCGSAMATGKFLLAYSPPGAGVPKNRKEAMLGTHMIWDIGLQSRCVLCVPWISQTHYRFVSKDSYTDAGFITCWYQTSIVVPAEVQNQSVILCFVSACNDFSVRLLRDSPFVTQTAFYQNDVQNAVERSIVRVADTLPSGPSNSESIPALTAAETGHTSQVVPSDTIQTRHVRNFHVRSESSVENFLSRSACVYIVEYKTQDTTPDKMYDSWVINTRQVAQLRRKLEFFTYVRFDVEVTFVITSVQDDSTRQNTDTPVLTHQIMYVPPGGPIPHAVDDYNWQTSTNPSVFWTEGNAPPRMSIPFMSVGNAYSNFYDGWSHFSQTGVYGFNTLNNMGKLYFRHVNDRTISPITSKVRIYFKPKHVKAWVPRPPRLCEYTHKDNVDYEPKGVTTSRTSITITNSKHMETHGAFGQQSGAAYVGNYRVVNRHLATHTDWQNCVWEDYNRDLLVSTTTAHGCDTIARCHCTTGVYFCASRNKHHPVVFEGPGLVEVQESGYYPKRYQSHVLLAAGLSEPGDCGGILRCEHGVIGIVTMGGEGVVGFADVRDLLWLEDDAMEQGVKDYVEQLGNAFGSGFTNQICEQVNLLKESLIGQDSILEKSLKALVKIISALVIVVRNHDDLITVTATLALIGCTSSPWRWLKQKVSQYYGISMAERQNNGWLKKFTEMTNACKGMEWIAIKIQKFIEWLKVKILPEVREKHEFLNRLKQLPLLESQIATIEQSAPSQSDQEQLFSNVQYFAHYCRKYAPLYAAEAKRVFSLEKKMSNYIQFKSKCRIEPVCLLLHGSPGVGKSVATNLIGRSLAEKLNSSIYSLPPDPDHFDGYKQQAVVIMDDLCQNPDGKDVSLFCQMVSSVDFVPPMAALEEKGILFTSPFVLASTNAGSINAPTVSDSRALARRFHFDMNIEVISMYNQNGKINMPMSVKTCDEECCPVNFKKCCPLVCGKAIQFIDRRTQVRYSLDMLVTEMFREYNHRHSVGATLEALFQGPPVYREIKISVAPEIPPPPAIADLLKSVDSEAVRDYCKEKGWLVPEVNSTLQIEKHVSRAFICLQALTTFVSVAGIIYIIYKLFAGFQGAYTGMPNQKPKVPTLRQAKVQGPAFEFAVAMMKRNSSTVKTEYGEFTMLGIYDRWAVLPRHAKPGPTILMNDQEVGVVDAKELVDKDGTNLELTLLKLNRNEKFRDIRGFLAKEEVEVNEAVLAINTSKFPNMYIPVGQVTDYGFLNLGGTPTKRMLMYNFPTRAGQCGGVLMSTGKVLGIHVGGNGHQGFSAALLKHYFNDEQGEIEFIESSKDAGYPVINTPSRTKLEPSVFHQVFEGSKEPAVLRNGDPRLKANFEEAIFSKYIGNVNTHVDEYMLEAIDHYAGQLATLDISTEPMKLEDAVYGTEGLEALDLTTSAGYPYVALGIKKRDILSKKTRDLTKLKECMDKYGLNLPMVTYVKDELRSAEKVAKGKSRLIEASSLNDSVAMRQTFGNLYKAFHQNPGIVTGSAVGCDPDLFWSKIPVMLDGHLIAFDYSGYDASLSPVWFACLKLLLEKLGYTHKETNYIDYLCNSHHLYRDKHYFVRGGMPSGCSGTSIFNSMINNIIIRTLMLKVYKGIDLDQFRMIAYGDDVIASYPWPIDASLLAEAGKDYGLIMTPADKGECFNEVTWTNVTFLKRYFRADEQYPFLVHPVMPMKDIHESIRWTKDPKNTQDHVRSLCLLAWHNGEQEYEEFIQKIRSVPVGRCLTLPAFSTLRRKWLDSF

SEQ ID NO: 177 - protein, 2185 aa, synthetic construct
MGAQVSTQKTGAHETSLSAAGNSVIHYTNINYYKDAASNSANRQDFTQDPGKFTEPVKDIMVKSMPALNSPSAEECGYSDRVRSITLGNSTITTQECANVVVGYGVWPTYLKDEEATAEDQPTQPDVATCRFYTLESVMWQQSSPGWWWKFPDALSNMGLFGQNMQYHYLGRAGYTIHVQCNASKFHQGCLLVVCVPEAEMGCATLANKPDPKSLSKGEIANMFESQNSTGETAVQANVINAGMGVGVGNLTIFPHQWINLRTNNSATIVMPYINSVPMDNMFRHNNFTLMVIPFAPLSYSTGATTYVPITVTVAPMCAEYNGLRLAGKQGLPTLSTPGSNQFLTSDDFQSPSAMPQFDVTPEMDIPGQVNNLMEIAEVDSVVPVNNTEGKVMSIEAYQIPVQSNPTNGSQVFGFPLTPGANSVLNRTLLGEILNYYAHWSGSIKLTFMFCGSAMATGKFLLAYSPPGAGAPTTRKEAMLGTHVIWDVGLQSSCVLCIPWISQTHYRYVVMDEYTAGGYITCWYQTNIVVPADAQSDCKILCFASACNDFSVRMLKDTPFIKQDNFFQGPPGEVVERAIARVADTIGSGPVNSESIPALTAAETGHTSQVVPSDTMQTRHVKNYHSRSESTVENFLCRSACVFYTTYKNHDSDGDNFAYWVINTRQVAQLRRKLEMFTYARFDLELTFVITSTQEQPTVRGQDTPVLTHQIMYVPPGGPVPTKVNSYSWQTSTNPSVFWTEGSAPPRMSIPFIGIGNAYSMFYDGWARFDKQGTYGTSTLNNMGTLYMRHVNDGGPGPIVSTVRIYFKPKHVKTWVPRPPRLCQYQKAGNVNFEPTGVTEGRTDITTMKTTGAFGQQSGAVYVGNYRVVNIHLATRADWQNCVWEDYDRDLLVSTTTAHGCDTIARCDCTAGVYFCASRNKHYPVTFEGPGLVEVQESEYYPKKHQSHVLLAAGFAEPGDCGGILRCQHGVIGIVTMGGEGVVGFADVRDLLWLEDDAMEQGVRDYVEQLGNAFGSGFTNQICEQVTLLKESLIGQDSILEKSLKALVKIVSALVIVVRNHDDLITVTATLALIGCTTSPWRWLKQKVSQYYGIPMAERQNSGWLKKFTEMTNACKGMEWIAIKIQKFIEWLKVKILPEVKEKHEFLNRLKQLPLLESQIATIEQSAPSQSDQEQLFSNVQYFAHYCRKYAPLYAAEAKRVFSLEKKMSNYIQFKSKCRIEPVCLLLHGSPGAGKSVATNLIGRSLAEKLNSSVYSLPPDPDHFDGYKQQAVVIMDDLCQNPDGKDVSLFCQMVSSVDFVPPMAALEEKGILFTSPFVLASTNAGSVNAPTVSDSRALVRRFHFDMNIEVVSMYSQNGKINMPMAVKTCDEECCPVNFKKCCPLVCGKAIQFIDRRTQVRYSLDMLVTEMFREYNHRHSVGATLEALFQGPPVYREIKISVAPETPPPPAVADLLKSVDSEAVREYCKEKGWLIPEVDSTLQIEKHVNRAFICLQALTTFVSVAGIIYIIYKLFAGFQGAYTGMPNQKPKVPTLRQAKVQGPAFEFAVAMMKRNASTVKTEYGEFTMLGIYDRWAVLPRHAKPGPTILMNDQVVGVLDAKELVDKDGTNLELTLLKLNRNEKFRDIRGFLAREEVEANEAVLAINTSKFPNMYIPVGRVTDYGFLNLGGTPTKRMLMYNFPTRAGQCGGVLMSTGKVLGIHVGGNGHQGFSAALLRHYFNEEQGEIEFVESSKDAGFPVINTPSKTKLEPSVFHHVFEGNKEPAVLRNGDPRLKANFEEAIFSKYIGNVNTHVDEYMMEAVDYYAGQLATLDISTEPMKLEDAVYGTEGLEALDLTTSAGYPYVALGIKKRDILSKKTRDLTKLKECMDKYGLNLPMVTYVKDELRSADKVAKGKSRLIEASSLNDSVAMRQTFGNLYKTFHLNPGIVTGSAVGCDPDVFWSKIPVMLDGHLIAFDYSGYDASLSPVWFTCLKLLLEKLGYTNKETNYIDYLCNSHHLYRDKHYFVRGGMPSGCSGTSIFNSMINNIIIRTLMLKVYKGIDLDQFRMIAYGDDVIASYPWPIDASLLAEAGKGYGLIMTPADKGECFNEVTWTNVTFLKRYFRADEQYPFLVHPVMPMKDIHESIRWTKDPKNTQDHVRSLCLLAWHNGEHEYEEFIRKIRSVPVGRCLSLPAFSTLRRKWLDSF

SEQ ID NO: 178 - protein, 2185 aa, synthetic construct
MGAQVSTQKTGAHETGLRASGNSIIHYTNINYYKDAASNSANRQEFAQDPGKFTEPVKDIMIKSMPALNSPSAEECGYSDRVRSITLGNSTITTQECANVVVGYGTWPTYLKDEEATAEDQPTQPDVATCRFYTLESVMWQQSSPGWWWKFPDALSNMGLFGQNMQYHYLGRAGYTVHVQCNASKFHQGCLLVVCVPEAEMGCATLANKPDQKSLSNGETANTFDSQNTTGQTAVQANVINAGMGVGVGNLTIFPHQWINLRTNNSATIVMPYINSVPMDNMFRHNNFTLMIIPFAPLSYSTGATTYVPITVTVAPMCAEYNGLRLAGKQGLPTMLTPGSNQFLTSDDFQSPSAMPQFDVTPEMAIPGQVNNLMEIAEVDSVVPVNNTEGKVSSIEAYQIPVQSNSTNGSQVFGFPLIPGASSVLNRTLLGEILNYYTHWSGSIKLTFMFCGSAMATGKFLLAYSPPGAGAPTTRKEAMLGTHVIWDVGLQSSCVLCIPWISQTHYRYVVVDEYTAGGYITCWYQTNIVVPADTQSDCKILCFVSACNDFSVRMLKDTPFIKQDSFYQGPPGEAVERAIARVADTISSGPVNSESIPALTAAETGHTSQVVPADTMQTRHVKNYHSRSESTVENFLCRSACVYYTTYKNHGTDGDNFAYWVINTRQVAQLRRKLEMFTYARFDLELTFVITSTQEQSTIQGQDSPVLTHQIMYVPPGGPVPTKINSYSWQTSTNPSVFWTEGSAPPRISIPFISIGNAYSMFYDGWAKFDKQGTYGINTLNNMGTLYMRHVNDGSPGPIVSTVRIYFKPKHVKTWVPRPPRLCQYQKAGNVNFEPTGVTESRTEITAMQTTGVLGQQTGAICIGNYRVVNRHLATSEDWQRCVWEDYNRDLLVSTTTAHGCDTIARCRCSTGVYFCASRNKHYPVSFEGPGLVEVQESEYYPKRYQSHVLLAAGFSEPGDCGGILRCEHGVIGLVTMGGEGVVGFADVRDLLWLEDDAMEQGVKDYVEQLGNAFGSGFTNQICEQVNLLKESLVGQDSILEKSLKALVKIISALVIVVRNHDDLVTITATLALIGCTSSPWRWLKQKVSQYYGIPMAERQNNNWLKKFTEMTNACKGMEWIAVKIQKFIDWLKVKILPEVKEKHEFLNRLKQLPLLESQIATIEQSAPSQSDQEQLFSNVQYFAHYCRKYAPLYAAEAKRVFSLEKKMSNYIQFKSKCRIEPVCLLLHGSPGAGKSVATNLIGRSLAEKLNSSVYSLPPDPDHFDGYKQQAVVIMDDLCQNPDGGDISLFCQMVSSVDFVPPMAALEEKGILFTSPFVLASTNAGSINAPTVSDSRALARRFHFDMNIEVISMYSQNGKINMPMSVRTCDEECCPVNFKRCCPLVCGKAIQFIDRRTQVRYSLDMLVTEMFREYNHRHSVGATLEALFQGPPIYREIKISVAPDTPPPPAIADLLKSVDSEAVREYCREKGWLVPEINSTLQIEKHVSRAFICLQALTTFVSVAGIIYIIYKLFAGFQGAYTGMPNQKPKVPTLRQAKVQGPAFEFAVAMMKRNSSTVKTEYGEFTMLGIYDRWAVLPRHAKPGPTILMNDQEVGVVDAKELVDKDGTNLELTLLKLSRNEKFRDIRGFLAKEEVEVNEAVLAINTSKFPNMYIPVGQVTDYGFLNLGGTPTKRMLMYNFPTRAGQCGGVLMSTGKVLGIHVGGNGHQGFSAALLKHYFNDEQGEIEFIESSKEAGLPVINTPSKTKLEPSVFHQVFEGNKEPAVLRNGDPRLKANFEEAIFSKYIGNVNTHVDEYMLEAVDHYAGQLATLDISTEPMKLEDAVYGTEGLEALDLTTSAGYPYVALGIKKRDILSKKTKDLTKLKECMDKYGLNLPMVTYVKDELRSAEKVAKGKSRLIEASSLNDSVAMRQTFGNLYKTFHLNPGIVTGSAVGCDPDLFWSKIPVLLDGHLIAFDYSGYDASLSPVWFACLKLLLEKLGYTHKETNYIDYLCNSHHLYRDKHYFVRGGMPSGCSGTSIFNSMINNIIIRTLMLKVYKGIDLDQFRMIAYGDDVIASYPWPIDASLLAEAGKDYGLIMTPADKGECFNEVTWTNVTFLKRYFRADEQYPFLVHPVMPMKDIHESIRWTKDPKNTQDHVRSLCLLAWHNGEHEYEEFIKKIRSVPVGRCLTLPAFSTLRRKWLDSF

SEQ ID NO: 179 - protein, 2193 aa, synthetic construct
MGAQVSTQKTGAHETGLSASGNSIIHYTNINYYKDAASNSANRQDFTQDPGKFTEPVKDIMIKSMPALNSPTAEECGYSDRVRSITLGNSTITTQECANVVVGYGTWPDYLHDDEATAEDQPTQPDVATCRFYTLESIQWQKTSDGWWWKFPEALKDMGLFGQNMHYHYLGRSGYTIHVQCNASKFHQGCLLVVCVPEAEMGCATVANEVNAAALSSGETAKHFAKTGATGTHTVQSIVTNAGMGVGVGNLTIFPHQWINLRTNNSATIVMPYINSVPMDNMFRHYNFTLMIIPFVPLDFTAEASTYVPITVTVAPMCAEYNGLRLASHQGLPTMNTPGSNQFLTSDDFQSPSAMPQFDVTPELRIPGEVKNLMEIAEVDSVVPVNNTQDSVYNMDVYKIPVSGGNQLSTQVFGFQMQPGLNSVFKRTLLGEILNYYAHWSGSVKLTFVFCGSAMALAKFLLAYSPPGADPPKSRKEAMLGTHVIWDIGLQSSCVLCVPWISQTHYRLVQQDEYTSAGYVTCWYQTSLVVPPGAPATCGVLCLASACNDFSVRMLRDTPFIEQKQLLQGDVEEAVNRAVARVADTLPTGPRNSESIPALTAAETGHTSQVVPGDTMQTRHVKNYHSRTESSVENFLCRAACVYITKYKTKDSDPVQRYANWRINTRQMVQLRRKFELFTYLRFDMEVTFVITSSQDDGTQLAQDMPVLTHQVMYIPPGGPVPNSVTDFAWQSSTNPSIFWTEGNAPARMSIPFISIGNAYSNFYDGWSHFTQDGVYGFNSLNNMGSIYIRHVNEQSPYAITSTVRVYFKPKHVRAWVPRPPRLCAYEKSSNVNFKPTDVTTSRTSITEVPSLRPSVVNTGAFGQQSGAAYVGNYRVVNRHLATHVDWQNCVWEDYNRDLLVSTTTAHGCDTIARCQCTTGVYFCASRNKHYPVSFEGPGLVEVQESEYYPRRYQSHVLLAAGFSEPGDCGGILRCEHGVIGLVTMGGEGVVGFADVRDLLWLEDDAMEQGVKDYVEQLGNAFGSGFTNQICEQVNLLKESLVGHDSILEKSLKALVKIISALVIVVRNHDDLITVTATLALIGCTSSPWRWLKHKVSQYYGIPMAERQSNGWLKKFTEMTNACKGMEWIAIKIQKFIEWLKLKILPEVKEKHEFLNRLKQLPLLESQIATIEQSAPSQSDQEQLFSNVQYFAHYCRKYAPLYAAEAKRVFSLEKKMSNYIQFKSKCRIEPVCLLLHGSPGAGKSVATSLIGRSLAEKLNSSVYSLPPDPDHFDGYKQQAVVIMDDLCQNPDGKDVSLFCQMVSSVDFVPPMAALEEKGILFTSPFVLASTNAGSINAPTVSDSRALARRFHFDMNIEVISMYSQNGKINMPMSVKTCDEECCPVNFKRCCPLVCGKAIQFIDRRTQVRYSLDMLVTEMFREYNHRHSVGATLEALFQGPPVIREIKISVAPETPPPPAIADLLKSVDSEAVREYCKEKGWLVPEVNSTLQIEKHVSRAFICLQALTTFVSVAGIIYIIYKLFAGFQGAYTGMPNQKPKVPTLRQAKVQGPAFEFAVAMMKRNASTVKTEYGEFTMLGIYDRWAVLPHHAKPGPTILMNDQEIGVLDAKELVDKDGTNLELTLLKLNRNEKFRDIRGFLAREEAEVNEAVLAINTSKFPNMYIPVGQVTDYGFLNLGGTPTKRMLMYNFPTRAGQCGGVLMSTGKVLGIHVGGNGHQGFSAALLRHYFNEEQGEIEFIESSKDAGFPVINTPSKTKLEPSVFHQVFEGNKEPAVLRNGDPRLKVNFEEAIFSKYIGNINTHVDEYMLEAVDHYAGQLATLDISTEPMKLEDAVYGTEGLEALDLTTSAGYPYVAIGIKKRDILSKKTKDLTKLKECMDKYGLNLPMVTYVKDELRSSEKVAKGKSRLIEASSLNDSVAMRQTFGNLYKTFHLNPGIVTGSAVGCDPDLFWSKIPVMLDGHLIAFDYSGYDASLSPVWFACLKLLLEKLGYTHRETNYIDYLCNSHHLYRDKHYFVRGGMPSGCSGTSIFNSMINNIIIRTLMLKVYKGIDLDQFRMIAYGDDVIASYPWPIDASLLAEAGKGYGLIMTPADKGECFNEVTWTNVTFLKRYFRADEQYPFLVHPVMPMKDIHESIRWTKDPKNTQDHVRSLCLLAWHNGEQEYEEFIRKIRSVPVGRCLTLPAFSTLRRKWLDSF

SEQ ID NO: 180 - protein, 2195 aa, synthetic construct
MGAQVSTQKTGAHETGLNASGSSIIHYTNINYYKDAASNSANRQEFSQDPGKFTEPVKDIMVKSLPALNSPSAEECGYSDRVRSITLGNSTITTQESANVVVGYGRWPEYLKDNEATAEDQPTQPDVATCRFYTLESVTWERDSPGWWWKFPDALKDMGLFGQNMYYHYLGRAGYTLHVQCNASKFHQGCLLVVCVPEAEMGCSQVDGTVNEHGLSEGETAKKFSSTSTNGTNTVQTIVTNAGMGVGVGNLTIYPHQWINLRTNNCATIVMPYINNVPMDNMFRHHNFTLMIIPFVPLDYSSDSSTYVPITVTVAPMCAEYNGLRLSTSLQGLPVMNTPGSNQFLTSDDFQSPSAMPQFDVTPELNIPGEVQNLMEIAEVDSVVPVNNVEGKLDTMEVYRIPVQSGNHQSDQVFGFQVQPGLDSVFKHTLLGEILNYFAHWSGSIKLTFVFCGSAMATGKFLLAYAPPGANAPKNRKDAMLGTHIIWDVGLQSSCVLCVPWISQTHYRLVQQDEYTSAGNVTCWYQTGIVVPAGTPTSCSIMCFVSACNDFSVRLLKDTPFIEQTALLQGDVVEAVENAVARVADTIGSGPSNSQAVPALTAVETGHTSQVTPSDTMQTRHVKNYHSRSESSIENFLSRSACVYMGGYHTTNTDQTKLFASWTISARRMVQMRRKLEIFTYVRFDVEVTFVITSKQDQGSRLGQDMPPLTHQIMYIPPGGPIPKSVTDYAWQTSTNPSIFWTEGNAPPRMSIPFISIGNAYSNFYDGWSHFSQNGVYGYNTLNHMGQIYVRHVNGSSPLPMTSTVRMYFKPKHVKAWVPRPPRLCQYKNASTVNFTPTNVTDKRTSINYIPETVKPDLSNYGAFGYQSGAVYVVNYRVVNRHLATHTDWQNCVWEDYNRDLLISTTTAHGCDVIARCRCSTGVYYCQSKGKHYPVNFEGPGLVEVQESEYYPKRYQSHVLLAAGFSEPGDCGGILRCEHGVIGIVTMGGEGVVGFADVRDLLWLEDDAMEQGVKDYVEQLGNAFGSGFTNQICEQVNLLKESLVGQDSILEKSLKALVKIISALVIVVRNHDDLITVTATLALIGCTSSPWRWLKQKVSQYYGIPMAERQNNGWLKKFTEMTNSCKGMEWISIKIQKFIEWLKVKILPEVREKHEFLNRLKQLPLLESQIATIEQSAPSQSDQEQLFSNVQYFAHYCRKYAPLYASEAKRVFSLEKKMSNYIQFKSKCRIEPVCLLLHGSPGAGKSVATNLIGRSLAEKLNSSVYTLPPDPDHFDGYKQQAVVIVDDLCQNPDGKDVSLFCQMVSSVDFVPPMAALEEKGILFTSLFVLASTNAGSINAPTVSDSRALARRFHFDMNIEVISMYSQNGKINMPMSEKTCDEECCPVNFKRCCPLVCGKAIQFIDRRTQVRYSLDMLVTEMFREYNHRHSVGATLEALFQGPPIYREIKISVAPETPPPPAIADLLKSVDSEAVREYCKEKGWLVPEVNSTLQIEKHVSRAFICLQALTTFVSVAGIIYIIYKLFAGFQGAYTGMPNQKPKVPTLRQAKVQGPAFEFAVAMMKRNSSTVKTEYREFTMLGIYDRWAVLPRHAKPGPTILMNNQEVGVLDAKELVDKDGTNLELTLLKLNRNEKFRDIRGFLAKEEVEANQAVLAINTSKFPNMYIPVGQVTDYGFLNLGGTPTKRMLMSNFPTRAGQCGGVLMSTGKVLGIHVGGNGHQGFSAALLKHYFNDEQGEIEFIESSKDAGFPIINTPSKTKLEPSVFHQVFEGDKEPAVLRNGDPRLKANFEEAIFSKYIGNVNTHVDEYMLEAVDHYAGQLATLDISTEPMRLEDAVYGTEGLEALDLTTSAGYPYVALGIKKRDILSRRTRDLTKLKECMDKYGLNLPMVTYVKDELRSADKVAKGKSRLIEASSLNDSVAMRQTFGNLYRTFHLNPGIVTGSAVGCDPDLFWSKIPVMLDGHLIAFDYSGYDASLSPVWFACLKLLLEKLGYTHKETNYIDYLCNSHHLYRDKHYFERGGMPSGYSGTSMFNSMINNIIIRTLMLKVYKGIDLDQFRMIAYGDDVIASYPWPIDASLLAETGKGYGLIMTPADKGECFNEVTWTNVTFLKRYFRADEQYPFLVHPVMPMKDIHESIRWTKDPKNTQDHVRSLCLLAWHNGEHEYEEFIRKIRSVPVGRCLTLPAFSTLRRKWLDSF

SEQ ID NO: 181 - protein, 2201 aa, synthetic construct
MGAQVSTQKTGAHETSLSAAGNSIIHYTNINYYKDAASNSANRQDFTQDPSKFTEPVKDVMIKSLPALNSPTVEECGYSDRVRSITLGNSTITTQECANVVVGYGRWPTYLRDDEATAEDQPTQPDVATCRFYTLDSIKWEKGSVGWWWKFPEALSDMGLFGQNMQYHYLGRAGYTIHLQCNASKFHQGCLLVVCVPEAEMGGAVVGQAFSATAMANGDKAYEFTSATQSDQTKVQTAIHNAGMGVGVGNLTIYPHQWINLRTNNSATIVMPYINSVPMDNMFRHYNFTLMVIPFVKLDYADTASTYVPITVTVAPMCAEYNGLRLAQAQGLPTMNTPGSTQFLTSDDFQSPCALPQFDVTPSMNIPGEVKNLMEIAEVDSVVPVNNVQDTTDQMEMFRIPVTINAPLQQQVFGLRLQPGLDSVFKHTLLGEILNYYAHWSGSMKLTFVFCGSAMATGKFLIAYSPPGANPPKTRKDAMLGTHIIWDIGLQSSCVLCVPWISQTHYRLVQQDEYTSAGYVTCWYQTGMIVPPGTPNSSSIMCFASACNDFSVRMLRDTPFISQDNKLQGDVEEAIERARCTVADTMRTGPSNSASVPALTAVETGHTSQVTPSDTMQTRHVKNYHSRSESTVENFLGRSACVYMEEYKTTDKHVNKKFVAWPINTKQMVQMRRKLEMFTYLRFDMEVTFVITSRQDPGTTLAQDMPVLTRQIMYVPPGGPIPAKVDDYAWQTSTNPSIFWTEGNAPARMSIPFISIGNAYSNFYDGWSNFDQRGSYGYNTLNNLGHIYVRHVSGSSPHPITSTIRVYFKPKHTRAWVPRPPRLCQYKKAFSVDFTPTPITDTRKDINTVTTVAQSRRRGDMSTLNTHGAFGQQSGAVYVGNYRVINRHLATHTDWQNCVWEDYNRDLLVSTTTAHGCDVIARCQCTTGVYFCASKNKHYPVSFEGPGLVEVQESEYYPKRYQSHVLLAAGFSEPGDCGGILRCEHGVIGIVTMGGEGVVGFADVRDLLWLEDDAMEQGVKDYVEQLGNAFGSGFTNQICEQVNLLKESLVGQDSILEKSLKALVKIISALVIVVRNHDDLITVTAILALIGCTSSPWRWLKQKVSQYYGIPMAERQNDSWLKKFTEMTNACKRMEWIAIKIQKFIEWLKVKILPEVREKHEFLNRLKQLPLLESQIATIEQSAPSQSDQEQLFSNVQYFAHYCRKYAPLYAAEAKRVFSLEKKMSNYIQFKSKCRIEPVCLLLHGSPGAGKSVATNLIGRSLAEKLNSSVYSLPPDPDHFDGYKQQAVVIMDDLCQNPDGKDVSLFCQMVSSVDFVPPMAALEEKGILFTSPFVLASTNAGSINAPTVSDSRALARRFHFDMNIEVISMYSQNGKINMPMSVKTCDEECCPVNFKKCCPLVCGKAIQFIDRRTQVRYSLDMLVTEMFREYNHRHSVGATLEALFQGPPIYREIKISVAPETPPPPVIADLLKSVDSEDVREYCKEKGWLIPEVNSTLQIEKYVSRAFICLQAITTFVSVAGIIYIIYKLFAGFQGAYTGIPNQKPKVPTLRQAKVQGPAFEFAVAMMKRNSSTVKTEYGEFTMLGIYDRWAVLPRHAKPGPTILMNDQEVGVMDAKELVDKDGTNLELTLLKLNRNEKFRDIRGFLAKEEMEVNEAVLAINTSKFPNMYIPVGQVTDYGFLNLGGTPTKRMLMYNFPTRAGQCGGVLMSTGKVLGIHVGGNGHQGFSAALLKHYFNDEQGEIEFIESSKDAGFPIINTPSKTKLEPSVFHQVFEGVKEPAVLRNGDPRLKANFEEAIFSKYIGNVNTHVDEYMLEAVDHYAGQLATLDISTEPMKLEDAVYGTEGLEALDLTTSAGYPYVALGIKKRDILSKKTRDLTKLKECMDKYGLNLPMITYVKDQLRSAEKVAKGKSRLIEASSLNDSVAMRQTFGNLYKTFHLNPGIVTGSAVGCDPDLFWSKIPVMLNGHLIAFDYSGYDASLSPVWFACLKLLLEKLGYSHKETNYIDYLCNSHHLYRDKHYFVRGGMPSGCSGTSIFNSMINNIIIRTLMLKVYKGIDLDQFRMIAYGDDVIASYPWPIDASLLAEAGKDYGLIMTPADKGECFNEVTWTNVTFLKRYFRADEQYPFLVHPVMPMKDIHESIRWTKDPKNTQDHVRSLCLLAWHNGEHEYEEFIRKIRSVPVGRCLTLPAFSTLRRKWLDSF

SEQ ID NO: 182 - protein, 2185 aa, synthetic construct
MGAQVSTQKTGAHETGLNASGNSIIHYTNINYYKDAASNSANRQDFTQDPSKFTEPVKDIMIKSLPALNSPTVEECGYSDRVRSITLGNSTITTQECANVVVGYGVWPDYLKDSEATAEDQPTQPDVATCRFYTLDSVQWQKTSPGWWWKLPDALSNLGLFGQNMQYHYLGRTGYTIHVQCNASKFHQGCLLVVCVPEAEMGCATLNNTPSSAELLGGDSAKEFADKPVASGSNKLVQRVVYNAGMGVGVGNLTIFPHQWINLRTNNSATIVMPYTNSVPMDNMFRHNNVTLMVIPFVPLDYCPGSTTYVPITITIAPMCAEYNGLRLAGHQGLPTMNTPGSCQFLTSDDFQSPSAMPQYDVTPEMRIPGEVKNLMEIAEVDSVVPVQNVGEKVNSMEAYQIPVRSNEGSGTQVFGFPLQPGYSSVFSRTLLGEILNYYTHWSGSIKLTFMFCGSAMATGKFLLAYSPPGAGAPTKRVDAMLGTHVVWDVGLQSSCVLCIPWISQTHYRYVASDEYTAGGFITCWYQTNIVVPADAQSSCYIMCFVSACNDFSVRLLKDTPFISQQNFFQGPVEDAITAAIGRVADTVGTGPTNSEAIPALTAAETGHTSQVVPSDTMQTRHVKNYHSRSESTIENFLCRSACVYFTEYENSGAKRYAEWVITPRQAAQLRRKLEFFTYVRFDLELTFVITSTQQPSTTQNQDAQILTHQIMYVPPGGPVPDKVDSYVWQTSTNPSVFWTEGNAPPRMSVPFLSIGNAYSNFYDGWSEFSRNGVYGINTLNNMGTLYARHVNAGSTGPIKSTIRIYFKPKHVKAWIPRPPRLCQYEKAKNVNFQPSGVTTTRQSITTMTNTGAFGQQSGAVYVGNYRVVNRHLATSADWQNCVWENYNRDLLVSTTTAHGCDIIARCRCTTGVYFCASKNKHYPISFEGPGIVEVQESEYYPRRYQSHVLLAAGFSEPGDCGGILRCEHGVIGIVTMGGEGVVGFADIRDLLWLEDDAMEQGVKDYVEQLGNAFGSGFTNQICEQVNLLKESLVGQDSILEKSLKALVKIISALVIVVRNHDDLITVTATLALIGCTSSPWRWLKQKVSQYYGIPMAERQNNGWLKKFTEMTNACKGMEWIAIKIQKFIEWLKVKILPEVREKHEFLNRLKQLPLLESQIATIEQSAPSQSDQEQLFSNVQYFAHYCRKYAPLYASEAKRVFSLEKKMSNYIQFKSKCRIEPVCLLLHGSPGAGKSVATNLIGRSLAEKLNSSVYSLPPDPDHFDGYKQQAVVIMDDLCQKPDGKDVSLFCQMVSSVDFVPPMAALEEKGILFTSPFVLASTNAGSINAPTVSDSRALARRFHFDMNIEVISMYSQNGKINMPMSVKTCDEECCPVNFKKCCPLVCGKAIQFIDRRTQVRYSLDMLVTEMFREYNHRHSVGATLEALFQGPPVYREIKISVAPETPPPPRIADLLKSVDSEAVREYCKEKGWLVPEVNSTLQIEKHVSRAFICLQAITTFVSVAGIIYIIYKLFAGFQGAYTGIPNQKPKVPTLRQAKVQGPAFEFAVAMMKRNSSTVKTEYGEFTMLGIYDRWAVLPRHAKPGPTILMNDQEVGVLDAKELVDKDGTNLELTLLKLNRNEKFRDIRGFLAKEEVEVNEAVLAINTSKFPNMYIPVGQVTDYGFLNLGGTPTKRMLMYNFPTRAGQCGGVLMSTGKVLGIHVGGNGHQGFSAALLKHYFNDEQGEIEFIESSKEAGFPIINTPSKTKLEPSVFHQVFEGDKEPAVLRNGDPRLKVNFEEAIFSKYIGNVNTHVDEYMMEAVDHYAGQLATLDISTEPMKLEDAVYGTEGLEALDLTTSAGYPYVALGIKKRDILSKKTRDLTKLKECMDKYGLNLPMVTYVKDELRSAEKVAKGKSRLIEASSLNDSVAMRQTFGNLYKTFHLNPGVVTGSAVGCDPDLFWSKIPVMLDGHLIAFDYSGYDASLSPVWFACLKLLLEKLGYSHKETNYIDYLCNSHHLYRDKHYFVRGGMPSGCSGTSIFNSMINNIIIRTLMLKVYKGIDLDQFRMIAYGDDVIASYPWPIDASLLAEAGKDYGLIMTPADKGECFNEVTWTNVTFLKRYFRADEQYPFLVHPVMPMKDIHESIRWTKDPKNTQDHVRSLCLLAWHNGEHEYEEFIRKIRSVPVGRCLTLPAFSTIRRKWLDSF

SEQ ID NO: 183 - protein, 2183 aa, synthetic construct
MGAQVSTQKTGAHETSLSATGNSIIHYTNINYYKDAASNSANRQDFTQDPSKFTEPVKDVMIKSLPALNSPTVEECGYSDRVRSITLGNSTITTQECANVVVGYGVWPDYLSDEEATAEDQPTQPDVATCRFYTLKSVKWEMQSAGWWWKFPDALSEMGLFGQNMQYHYLGRSGYTIHVQCNASKFHQGCLLVVCVPEAEMGCTNAENAPTYGDLCGGETAKQFEQNAVTGETAVQTAVCNAGMGVGVGNLTIYPHQWINLRTNNSATIVMPYINSVPMDNMFRHNNFTLMIIPFAPLDYVTGASSYIPITVTVAPMSAEYNGLRLAGHQGLPTMLTPGSTQFLTSDDFQSPSAMPQFDVTPEMNIPGQVRNLMEIAEVDSVVPINNLQANLKTMEAYRVQVRSTDEMGGQIFGFPLQPGASSVLQRTLLGEILNYYTHWSGSLKLTFVFCGSAMATGKFLLAYSPPGAGAPDSRKNAMLGTHVIWDVGLQSSCVLCVPWISQTHYRYVVDDKYTASGFISCWYQTNVIVPAEAQKSCYIMCFVSACNDFSVRMLRDTQFIKQDTFYQGPTEESVERAMGRVADTIARGPSNSEQIPALTAVETGHTSQVDPSDTMQTRHVHNYHSRSESSIENFLCRSACVIYIKYSSAESNNLKRYAEWVINTRQVAQLRRKMEMFTYIRCDMEQTFVITSHQEMSTATNSVVPVQTHQIMYVPPGGPVPTSVNDYVWQTSTNPSIFWTEGNAPPRMSIPFMSIGNAYTMFYDGWSNFSRDGIYGYNSLNNMGTIYARHVNDSSPGGLTSTIRIYFKPKHVKAYVRPPRRLCQYKKAKNVNFDVEAVTTERASLVTTGPHGQQSGAVYVGNYRVVNRHLATHFDWQNYIWEDYNRDLLVSTTTAHGCDTIARCQCTSGVYFCVSRNKHYPVVFEGPGLVEVQESEYYPKRYQSHVLLARGFSEPGDCGGILRCEHGVIGIRTMGREGVVGFADVRDLLWLEDDAMEQGVKDYVEELGNSFGSGFTNQICEQVNLLKESLVGQDSILEKSLKALVKIISALVIVVRNHDDLVTVTATLALIGCTTSPWRWLKRKVSQYYGIPMAERQNNNWLKKFTEMTNACKGMEWIAVKIQKFIEWLKVKILPEVKEKHEFLNRLKQLPLLESQIATIEQSAPSQSDQEQLFSNVQYFAHYCRKYAPLYAAEAKRVFSLEKKMSNYIQFKSKCRIEPVCLLLHGSPGAGKSVATNLIGRSLAEKLNSSVYSLPPDPDHFDGYKQQAVVIMDDLCQNPDGKDVSLFCQMVSSVDFVPPMAALEEKGILFTSPFVLASTNAGSINAPTVSDSRALARRFHFDMNIEVISMYSQNGKINMPMSVKTCDEECCPVNFKKCCPLVCGKAIQFIDRRTQVRYSLDMLVTEMFREYNHRHSVGATLEALFQGPPIYREIRISVAPETPPPPAIADLLRSVDSEAVREYCKEKGWLVPEISSTLQIEKHVSRAFICLQALTTFVSVAGIIYIIYKLFAGFQGAYTGMPNQKPKVPTFRQAKVQGPAFEFAVAMMKRNASTVKTEYGEFTMLGIYDRWAVLPRHAKPGPTILMNDQEVGVVDAKELVDKDGTTLELTLLKLNRNEKFRDIRGFLAREEAEVNEAVLAINTSKFPNMYIPVGQVTEYGFLNLGGTPTKRMLMYNFPTRAGQCGGVLMSTGKVLGIHVGGNGHQGFSASLLRHYFNDEQGEIEFIESSKEAGFPVINTPNKTKLEPSVFHHIFEGNKEPAVLRNGDTRLKVNFEEAIFSKYIGNVNTHVDEYMMEAVDHYAGQLATLDISTEPMRLEDAVYGTEGLEALDLTTSAGYPYVTLGIKKRDILSKKTKDLTKLKECMDKYGLNLPMVTYVKDELRSAEKVAKGKSRLIEASSLNDSVAMRQTFGNLYKTFHLNPGIVTGSAVGCDPDLFWSKIPVMLDGHLRAFDYSGYDASLSPVWFACLKLLLEKLGYSHKETNYIDYLCNSHHLYRDKHYFVRGGMPSGCSGTSIFNSMINNIIIRTLMFKVYKGIDLDQFRMIAYGDDVIASYPLPIDASLLAEAGKGYGLIMTPADKGECFNELTWTNVTFLKRYFRADEQYPFLVHPVMPIKDIHESIRWTKDPKNTQYHVRSLCLLAWHNGEQEYEELYPKIRSVPVGRCLTLPAFSTLRRKWLDAF

SEQ ID NO: 184 - protein, 2193 aa, synthetic construct
MGAQVSTQKTGAHEASLSATGSSIIHYTNINYYKDSASNSANRQDFTQDPSKFTEPVKDVMIKSLPALNSPSAEECGFSDRVRSITIGNSTITTQECANVVVAYGRWPTYLRDDEATAEDQPTQPDVATCRFYTLESVQWQENSAGWWWKFPDALSNMGLFGQNMLYHYLGRAGYTIHVQCNASKFHQGCLLVVCVPEAEMGCSKVDGVVNAQGLSKGEIPIEFERTSTTAEVGVVQKAVYNAGMGIGVGNLTIFPHQWINLRTNNSATIVIPYINCVPMDNMFRHNNFTLMVIPFAPLKSSGGTNYVPVTITVAPMDAEYNGLRLAGHQGLPTMNTPGSTQFLTSDDFQSPCAMPEFDVTPCMDIPGKVHNLMEIAEVDIGVPVNNTSTHMEGTDAFQIKVTAGNVQDKNAIFSFQLNPGNSTVLRRTLLGEILNYYAHWSGSIKLTFLFCGSAMATGKLLLAYSPPGASVPKARRDAMLGTHVIWDVGLQSSCVLCVPWISQTHYRLVAQDEYTSAGYITCWYQTNIVVPPETPSDCVVLCFVSACNDFSVRMLKDTPFIEQTTELQSDVREAVEGAIGRVADTIRSGPSNSEAVPALTAAETGHTSQVVPSDTMQTRHVKNYHSRSESTIENFLCRSACVRMAKYEARGNLKALTLDAWEISVRDMVQLRRKCEMFTYLRFDVEVTFVITSYQRQGTSSIQICPYDAHQIMYIPPGGPIPKKVDGYEWQTSTNPSIFWTEGNAPPRMSIPFISIGNAYSSFYDGWSHFDSKGAYGFNTLNKMGHIYCRHVNKETPTKVTSYIRIYFKPKHVRAWVPRPPRLCQYMNKANVNFEATAVTDTRDTINTVPLSTHGVSRGAYGHQSGARYVGNYRIVNRHLATHTDWQKCVWEDYNRDLLVSTTTAHGCDTIARCQCTTGVYFCASKNKHYPVSFEGPGLVEVQASEYYPKRYQSHVLLAAGFSEPGDCGGILRCEHGVIGIVTMGGEGVVGFADVRDLLWLEDDAMEQGVKDYVEQLGNAFGSGFTNQICEQVNLLKESLVGQDSILEKSLKALVKIISALVIVVRNHDDLITVTAVLALIGCTTSPWRWLKQKVSQYYGIPMAERQNNRWLKKFTEMTNACKGMEWIAIKIQKFIEWLKIKILPEVKEKHEFLNRLKQLPLLESQIATIEQSAPSQGDQEQLFSNIQYFAHYCRKYAPLYAAEAKRVFSLEKKMSNYIQFKSKCRIEPVCLLLHGSPGAGKSVATNLIGRSLAEKLNSSVYSLPPDPDHFDGYKQQAVVIMDDLCQNPDGKDVSLFCQMVSSVDFVPPMAALEEKGILFTSPFVLASTNAGSINAPTVSDSRALARRFHFDMNIEVISMYSQNGKINMPMSVKTCDEECCPVNFKKCCPLVCGKAIQFIDRRTQVRYSLDMLVTEMFREYNHRHSVGTTLEALFQGPPIYREIKISVAPETPPPPAIADLLKSVDSEAVREYCKEKGWLVPEVNSTLQIEKHVSRAFICLQALTTFVSVAGIIYIIYKLFAGFQGAYTGMPNQKPRVPTLRQAKVQGPAFEFAVAMMKRNSSTVKTEYGEFTMLGIYDRWAVLPRHAKPGPTILMNDQEVGVLDAKELVDKDGTNLELTLLKLNRNEKFRDIRGFLAKEEVEVNEAVLAINTSKFPNMYIPVGQVTDYGFLNLGGTPTKRMLMYNFPTRAGQCGGVLMSTGKVLGIHVGGNGHQGFSAALLKHYFNDEQGEIEFIESSKEAGFPVINTPSKTKLEPSVFHQVFEGNKEPAVLRNGDPRLKANFEEALFSKYIGNVNTHVDEYMLEAVDHYAGQLATLDISTEPMRLEDAVYGTEGLEALDLTTSAGYPYVALGIKKRDILSKKTRDLTKLKECMDKYGLNLPMVTYVKDELRSAEKVAKGKSRLIEASSLNDSVAMRQTFGNLYKTFHLNPGIVTGSASGCDPDLFWSKIPVMLDGHLIAFDYSGYDASLSPVWFACLKLLLEKLGYSHKETNYIDYLCNSHHLYRDKHYFVRGGMPSGCSGTSIFNSMINNIIIRTLMLKVYKGIDLDQFRMIAYGDDVIASYPWPIDASLLAEAGKDYGLIMTPADKGECFNEVTWTNVTFLKRYFRADEQYPFLVHPVMPMKDIHESIRWTKDPKNTQDHVRSLCLLAWHNGEREYEEFIRKIRSVPVGRCLTLPAFSTLRRKWLDSF

SEQ ID NO: 185 - protein, 2194 aa, synthetic construct
MGAQVSTQKTGAHETGLSASGNSVIHYTNINYYKDSASNSLNRQDFTQDPSRFTEPVQDVLIKTLPALNSPTVEECGYSDRVRSITLGNSTITTQECANVVVGYGVWPTYLSDHEATAVDQPTQPDVATCRFYTLESVKWESSSAGWWWKFPEALSDMGLFGQNMQYHYLGRAGYTIHVQCNASKFHQGCLLVVCVPEAEMGAATTDHAMNHTKLSNIGQAMEFSAGKSTDQTGPQTAVHNAGMGVAVGNLTIYPHQWINLRTNNSATIVMPYINSVPMDNMYRHYNFTLMVIPFAKLEHSPQASTYVPITVTVAPMCAEYNGLRLAGHQGLPTMNTPGSTQFLTSDDFQSPSAMPQFDVTPEIQIPGQVRNLMEIAEVDSVVPVDNTEEHVNSIEAYRIPVRPQTNSGEQVFGFQLQPGYDSVLKHTLLGEILNYYANWSGSMKLTFMYCGAAMATGKFLIAYSPPGAGVPGSRKDAMLGTHVIWDVGLQSSCVLCVPWISQTNYRYVTRDAYTDAGYITCWYQTSIVTPPDIPTTSTILCFVSACNDFSVRLLRDTPFITQQALYQNDPEGALNKAVGRVADTIASGPVNTEQIPALTAVETGHTSQVVPSDTMQTRHVVNFHTRSESSLENFMGRAACAYIAHYTTEKANDDLDRYTNWEITTRQVAQLRRKLEMFTYMRFDLEITFVITSSQRTSNRYASDSPPLTHQIMYVPPGGPIPKGYEDFAWQTSTNPSVFWTEGNAPPRMSIPFMSVGNAYCNFYDGWSHFSQSGVYGYTTLNNMGHLYFRHVNKSTAYPVNSVARVYFKPKHVKAWVPRAPRLCPYLYAKNVNFDVQGVTESRGKITLDRSTHNPVLTTGAFEQQSGAAYVGNYRLVNRHLATHTDWQNCVWKDYNRDLLVSTTTAHGCDTIARCQCTTGVYFCASRNKHYPVTFEGPGLVEVQESEYYPKRYQSHVLLAAGFSEPGDCGGILRCEHGVIGLVTMGGEGVVGFADVRDLLWLEDDAMEQGVKDYVEQLGNAFGSGFTNQICEQVNLLKESLIGQDSILEKSLKALVKIISALVIVVRNHDDLITVTATLALIGCTTSPWRWLKHKVSQYYGIPMAERQNNNWLKKFTEMTNACKGMEWIAIKIQKFIEWLKVKILPEVKEKHEFLNRLKQLPLLESQIATIEQSAPSQSDQEQLFSNVQYFAHYCRKYAPLYAAEAKRVFSLEKKMSNYIQFKSKCRIEPVCLLLHGSPGAGKSVATNLIGRSLAEKLNSSVYSLPPDPDHFDGYKQQAVVIMDDLCQNPDGKDVSLFCQMVSSVDFVPPMAALEEKGILFTSPFVLASTNAGSINAPTVSDSRALARRFHFDMNIEVISMYSQNGKINMPMSVKTCDEECCPVNFKRCCPLVCGKAIQFIDRRTQVRYSLDMLVTEMFREYNHRHSVGATLEALFQGPPVYREIKISVAPETPPPPAIADLLKSVDSEAVREYCKEKGWLVPEINSTLQIEKHVSRAFICLQALTTFVSVAGIIYIIYKLFAGFQGAYSGMPNQKSKVPTLRQAKVQGPAFEFAVAMMKRNASTVKTEYGEFTMLGIYDRWAVLPRHAKPGPTIIMNDQEVGVVDAKELVDKDGTNLELTLLKLNRNEKFRDIRGFLAREEAEVNEAVLAINTSKFPNMYIPVGQVTDYGFLNLGGTPTKRMLMYNFPTRAGQCGGVLMSTGKVLGVHVGGNGHQGFSAALLRHYFNDEQGEIEFIESSKEAGFPVINTPSKTKLEPSVFHHVFEGNKEPAVLRNGDPRLKANFEEAIFSKYIGNVNTHVDEYMMEAVDHYAGQLATLDISTEPMKLEDAVYGTEGLEALDLTTSAGYPYVALGIKKRDILSKKTKDLAKLKECMDKYGLNLPMVTYVKDELRSAEKVAKGKSRLIEASSLNDSVAMRQTFGNLYKTFHMNPGIVTGSAVGCDPDLFWSKIPVMLDGHLIAFDYSGYDASLSPVWFACLKLLLEKLGYSHKETNYIDYLCNSHHLYRDKHYFVRGGMPSGCSGTSIFNSMINNIIIRTLMLKVYKGIDLDQFRMIAYGDDVIASYPHPIDASLLAEAGKGYGLIMTPADKGECFNEVTWTNVTFLKRYFRADEQYPFLVHPVMPMKDIHESIRWTKDPKNTQDHVRSLCLLAWHNGEQEYEEFVSKIRSVPVGRCLTLPAFSTLRRKWLDSF

SEQ ID NO: 186 - protein, 156 aa, synthetic construct
DPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTK

SEQ ID NO: 187 - protein, 156 aa, synthetic construct
DPLLVGQHVLKWIDKKCGKKNTLWFYGPPSTGKTNLAMAIAKSVPVYGMVNWNNENFPFNDVAGKSLVVWDEGIIKSTIVEAAKAILGGQPTRVDQKMRGSVAVPGVPVVITSNGDITFVVSGNTTTTVHAKALKERMVKLNFTVRCSPDMGLLTE

SEQ ID NO: 188 - protein, 156 aa, synthetic construct; identical to SEQ ID NO: 187

SEQ ID NO: 189 - protein, 156 aa, synthetic construct
DPAYAGSVFLGWAQKKFGKRNTIWLFGPATTGKTNIAEAIAHAVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLEHDFGKVTK

SEQ ID NO: 190 - protein, 156 aa, synthetic construct; identical to SEQ ID NO: 189

SEQ ID NO: 191 - protein, 156 aa, synthetic construct; identical to SEQ ID NO: 189

SEQ ID NO: 192 - protein, 156 aa, synthetic construct; identical to SEQ ID NO: 189

SEQ ID NO: 193 - protein, 156 aa, synthetic construct; identical to SEQ ID NO: 189

SEQ ID NO: 194 - protein, 156 aa, synthetic construct
EPAYAGSVFLGWAQKRFGKRNTIWLFGPATTGKTNIAEAIAHAVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLEHDFGKVTK

SEQ ID NO: 195 - protein, 156 aa, synthetic construct
DPAYAGSVFLGWAQKRFGKRNTIWLFGPATTGKTNIAEAIAHAVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLEHDFGKVTK

SEQ ID NO: 196 - protein, 156 aa, synthetic construct
DPQYAASVFLGWAQKKFGKRNTIWLFGPATTGKTNIAEAIAHAVPFYGCVNWTNENFPFNDCVDKMVIWWEEGKMTAKVVESAKAILGGSKVRVDQKCKASAQIDPTPVIVTSNTNMCAVIDGNSTTFEHQQPLQDRMFKFELTRRLDHDFGKVTK

SEQ ID NO: 197 - protein, 156 aa, synthetic construct
DPQYAASVFLGWAQKKFGKRNTIWLFGPATTGKTNIAEAIAHAVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTKRLEHDFGKVTK

SEQ ID NO: 198 - protein, 156 aa, synthetic construct; identical to SEQ ID NO: 197

SEQ ID NO: 199 - protein, 156 aa, synthetic construct
DPQYAASVFLGWAQKKFGKRNTIWLFGPATTGKTNIAEAIAHAVPFYGCVNWTNENFPFNDCVDKMVIWWEEGKMTAKVVESAKAILGGSKVRVDQKCKSSAQIEPTPVIVTSNTNMCAVIDGNSTTFEHQQPLQDRMFEFELTRRLDHDFGKVTK

SEQ ID NO: 200 - protein, 156 aa, synthetic construct
DPQYAASVFLGWAQKKFGKRNTIWLFGPATTGKTNIAEAIAHAVPFYGCVNWTNENFPFNDCVDKMVIWWEEGKMTAKVVESAKAILGGSKVRVDQKCKSSAQIEPTPVIVTSNTNMCAVIDGNSTTFEHQQPLQDRMFKFELTRRLDHDFGKVTK

SEQ ID NO: 201 - protein, 156 aa, synthetic construct
DPAYAGSILYGWCQRSFNKRNTVWLYGPATTGKTNIAEAIAHTVPFYGCVNWTNENFPFNDCVDKMLIWWEEGKMTNKVVESAKAILGGSKVRVDQKCKSSVQIDSTPVIVTSNTNMCVVVDGNSTTFEHQQPLEDRMFKFELTKRLPPDFGKITK

SEQ ID NO: 202 - protein, 156 aa, synthetic construct
NPWQVGHWLVMMLSKKTGKRNSTLFYGPASTGKTNLAKAICHAVGLYGCVNHNNKQFPFNDAPNKMILWWEECIMTTDYVEAAKCVLGGTHVRVDVKHKDSRELPQIPVLLSSNHDVYTVVGGNATFGVHAAPLKERITQMNFMKQLPNTFGEITP

SEQ ID NO: 203 - protein, 156 aa, synthetic construct
NPLAVGHALCCVLNKQFGKQNTVCFYGPASTGKTNMAKAIVQGIRLYGCVNHLNKGFVFNDCRQRLVVWWEECLMHQDWVEPAKCILGGTECRIDVKHRDSVLLTQTPVIISTNHDIYAVVGGNSVSHVHAAPLKERVIQLNFMKQLPQTFGEITA

SEQ ID NO: 204 - protein, 163 aa, synthetic construct
IFRMHGWNWIKVCHAIACVLNRQGGKRNTVLFHGPASTGKSIIAQAIAQAVGNVGCYNAANVNFPFNDCTNKNLIWIEEAGNFGQQVNQFKAICSGQTIRIDQKGKGSKQIEPTPVIMTTNENITIVRIGCEERPEHTQPIRDRMLNIKLVCKLPGDFGLVDK

SEQ ID NO: 205 - protein, 156 aa, synthetic construct
NYIKVCHAICCVLNRQGGKRNTVLFHGPASTGKSIIAQAIAQAVGNVGCYNAANVNFPFNDCTNKNLIWVEEAGNFGQQVNQFKAICSGQTIRIDQKGKGSKQIEPTPVIMTTNENITVVRIGCEERPEHTQPIRDRMLNIHLTRTLPGDFGLVDK

SEQ ID NO: 206 - protein, 156 aa, synthetic construct
NYIKVCHAITCVLNRQGGKRNTILFHGPASTGKSIIAQHIANLVGNVGCYNAANVNFPFNDCTNKNLIWIEEAGNFSNQVNQFKAICSGQTIRIDQKGKGSKQIEPTPVIMTTNEDITKVRIGCEERPEHTQPIRDRMLNINLTRKLPGDFGLLEE

SEQ ID NO: 207 - protein, 156 aa, synthetic construct
LNEQYLKKVLCTILTKQGGKRGCIWFYGPGGTGKTLLASLICKATVNYGMVTTSNPNFPWTDCGNRNIIWAEECGNFGNWVEDFKAITGGGDVKVDTKNKQPQSIKGCVIVTSNTNITKVTVGCVETNAHAEPLKQRMIKIRCMKTINPKTKITPG

SEQ ID NO: 208 - protein, 175 aa, synthetic construct
TVKRRNEILTNVMPKFARQEPFVVLFQGPGGIGKTHLVQQLATKCVNSFYQDHEDDYIEISPDDKYWPPLSGQRVAFFDEAGNLNDLTEDLLFRNIKSICSPAYFNCAAADIEHKISPCPFELVFATVNTDLDTLQSKISSTFGQASVFPIWRRCIVVECSWNEKELGPFNYKNP

SEQ ID NO: 209 - protein, 180 aa, synthetic construct
  MOD_RES 88: note = XXXXX
MRYLTEVFKSVDTSGAFGNKPRTQPIVIWLFGESGRGKSGMTWPLAIDLNNSLLDNVDEMRNFSKNIYMRNVEQEFWDNYQGQNIVCXDDFGQMRDSSSNPNPEFMELIRTANIAPYPLHMAHLEDKRKTKFTSKVIIMTSNVFEQDVNSLTFPDAFRRRVDLCAEVKNKDEFTKMCWSK

SEQ ID NO: 210 - protein, 175 aa, synthetic construct
AKSLYEQVLLSPVKGGGPKMRPITVWLTGESGIGKTQMIYPLCIDILREMGIVKPDAYKHQAYARQVETEYWDGYNGQKIVIYDDAFQLKDDKTKPNPEIFEVIRTCNTFPQHLHMAALQDKNMYSQAEVLLYTTNQFQVQLESITFPDAFYNRMKTHAYRVQIKQEKSIWVRNA

SEQ ID NO: 211 - protein, 175 aa, synthetic construct
AKQLYEYVSCSPVKGGGPRMRPICLWLVGESGVGKTEMVYPLCIDVLREMGMIKKDDFHHQVYGRQVETEFWDGYKGQKIVIYDDAFQKKDDKTAANPEIFEVIRSCNTFPQHLHMAALHDKNTFSAAELLLYTTNDYNVKLESITFPDAFFNRMGDMAYKVSPKKEYGIETEKG

SEQ ID NO: 212 - protein, 172 aa, synthetic construct
LQRAIDLLGQQGTMQKSKPVPFCLWVYGHSGCGKSHVCDNVLTEIGSALGINTANPIYTRSPDVEFWNGYTGQKLISWQDFAKITTGETYRKQVSELSSLIEPTPFNPPFAALEDKRKIADAWAVYVSSNKAFPEVQNMGMEDSALFYRRRHALVKMRINPEIIQEYARREP

SEQ ID NO: 213 - protein, 160 aa, synthetic construct
SSKIMELSTINAAALSAARKRVPAKIVVLCGPPGHGKSVAAHKLAKMLNPNEPSIWNPFSDHHDEYTAEDVVIIDETPAEPGQWVEDLIAMGSNSPFVPNYDRVENKTRCFDSKYVLITTNHNPLINPTHSRAAALARRLTWVYVNSPDVADFLRQHPGV

SEQ ID NO: 214 - protein, 160 aa, synthetic construct
SNKIVELSTMNASALSAARKRTPAKIIVLCGPPGHGKSVAAHKLAKMLNPNEPSIWNPFSDHHDEYTAEEVMVIDETPAEPGQWIEDLIAMGSNSPFVPNYDRVENKTRCFDSKYVIITTNHNPLINPTHTRAAALARRLTLVYVNSPDVADFLRQHPGV

SEQ ID NO: 215 - protein, 157 aa, synthetic construct
DSVISHCAKRKALATQRTAPVCIILTGPAGCGKTTLAYAIANRLSAQKPSVLNLNIDHHDAYTGNEVCIIDEFDSNPDSKFVEFVVEMVNTNPMLLNCDLIENKGKTFSSKYVIMTSNNETPVKPNSTRAPPFYRRVRIIDVTNPGVMSFKYENPGQ

SEQ ID NO: 216 - protein, 157 aa, synthetic construct
DGVITSCNKRKAIARKRQVPVCYILTGPPGCGKTTAAQALAKKLSDQEPSVINLDVDHHDTYTGNEVCIIDEFDSSDKVDYANFVIGMVNSAPMVLNCDMLENKGKLFTSKYIIMTSNSETPVKPSSKRAGAFYRRVTIIDVTNPFVESHKRARPGT

SEQ ID NO: 217 - protein, 157 aa, synthetic construct
DGVITSCNKRKAIAKKRPVPVCYILTGPPGCGKTTAALALAKKLSDQEPSVINLDVDHHDTYTGNEVCIVDEFDSSDKVDYANFVIGMVNSAPMVLNCDMLENKGKLFTSKYIIMTSNSETPVKPSSRRAGAFYRRVTIIDVANPLAESHKRARPGT

SEQ ID NO: 218 - protein, 157 aa, synthetic construct
DNVISCCTRRKAIATKRTAPVAVILTGPPGCGKTTAAFALAKRLSQQKPSIISLDVDHHDTYTGNEVCIIDEFDSSDKVDYANFVVNMVNTNPMVLNCDLVENKGKTFRSKYVIMKSNSETPVKPTSRRAGAFYRRVMIVDVKNTAVENWKRENPGK

SEQ ID NO: 219 - protein, 55 aa, synthetic construct
NKGKTFTSKYVIMTSNTETPVKPTSRRAGAFYRRVMIVDVTNNAVEKWKSDNPGK

SEQ ID NO: 220 - protein, 157 aa, synthetic construct
DKVISCCTRRKAIATKRTAPVAVILTGPPGCGKTTAAFALAKRLSQQKPSIISLDVDHHDTYTGNEVCIIDEFDSSDKVDYANFVVNMVNTNPMVLNCDLIENKGKTFTSKYVIMTSNTETPVKPTSRRAGAFYRRVMIVDVTNNAVDKWKSDNPGK

SEQ ID NO: 221 - protein, 155 aa, synthetic construct
QTTINELAQLMAYDTQRKPPAMVVFGGPPGIGKTRLVEALAKQLGEVSHFTMTVDHYDTYTGNTVAIWDEFDVDSKQAFIEATIGIVNCAPYPLNCDRPEAKGRVFTSKYVLATTNCPTPVMPDHPRAMAFWRRITFIDVTAPTIEQWLVDNPGR

SEQ ID NO: 222 - protein, 156 aa, synthetic construct
LESTANSIRSTILLDTPRKTPVAIILTGPPGIGKTRLAQHLAAGFGKVSNFSVTLDHHDSYTGNEVAIWDEFDVDTQGKFVETMIGVVNTAPYPLNCDRVENKGKVFTSDYIICTSNYPTSVLPDNPRAGAFYRRVTTIDVSSPTIEDWKKKNPGK

SEQ ID NO: 223 - protein, 155 aa, synthetic construct
ETQANNIRSTIKLDTPRRVPVVIILTGAPGIGKTRLAQYIGQRFGKTSNFSVAVDHHDGYTGNTVCIWDEFDVDSKGAFVETMIGIANTAPFPLNCDRVENKGRVFTSDYVICTSNYPTSVIPDNPRAAAFYRRVLTVDVSAPDLEEWKKRNPGK

SEQ ID NO: 224 - protein, 155 aa, synthetic construct
ETQANNIRSTIKLDTPRRVPVVIILTGAPGIGKTRLAQYVGQRFGKTSNFSVAVDHHDGYTGNTVCIWDEFDVDSKGAFVETMIGIANTAPFPLNCDRVENKGRVFTSDYVICTSNYPTSVIPDNPRAAAFYRRVLTVDVSAPDLEEWKKRNPGK

SEQ ID NO: 225 - protein, 161 aa, synthetic construct
QKVVADIHTLVLGKINMTKQRPQPVAVVFKGAPGIGKTYLVHRLAKDLGCPHPSNINFGLDHFDSYTGEDVAIADEFNTSGDERWVELFIQMVNTNPCPLNCDKVENKNKVFSSKYLLCTTNSSMVLNATHPRATAFYRRVIIVDVRNKAVEGWQSTRHGS

SEQ ID NO: 226 - protein, 162 aa, synthetic construct
QKVISDLHTMVLGKINTSKQRPQPVAVIFKGAPGIGKTYLVHRIARDLGCQHPSTINFGLDHFDSYTGEEVAIADESNTCGDGESWVELFIQMVNTNPCPLNCDKAENKNKVFNSKYLLCTTNSNMILNATHPRAGAFYRRVMIVEARNKAVESWQATRHGS

SEQ ID NO: 227 - protein, 162 aa, synthetic construct
QKVISDLHTMVLGKINMTKQRPQPVAVIFKGAPGIGKTYLVHRIARDLGCQHPSTINFGLDHFDSYTGEEVAIADEFNTCGDGESWVELFIQMVNTNPCPLNCDKAENKNKVFNSKYLLCTTNSNMILNATHPRAGAFYRRVMIVEARNKAVESWQATRHGS

SEQ ID NO: 228 - protein, 168 aa, synthetic construct
LARIAAARSLVHRAKEELSSRLRPVVVMISGKPGIGKTHLARELAKRIAASLTGDQRVGLIPRNGVDHWDAYKGERVVLWDDYGMSNPIHDALRLQELADTCPLTLNCDRIENKGKVFDSDAIIITTNLANPAPLDYVNFEACSRRIDFLVYAEAPEVEKAKRDFPGQ

SEQ ID NO: 229 - protein, 166 aa, synthetic construct
RISMARAALAKAQAEMTSRMRPVVIMMCGPPGIGKTKAAEHLAKRLANEIRPGGKVGLVPREAVDHWDGYHGEEVMLWDDYGMTKIQEDCNKLQAIADSAPLTLNCDRIENKGMQFVSDAIVITTNAPGPAPVDFVNLGPVCRRVDFLVYCTAPEVEHTRKVSPGD

SEQ ID NO: 230 - protein, 166 aa, synthetic construct
RISMARSALAKAQAEMTSRMRPVVIMMCGPPGIGKTKAAEHLAKRLANEIRPGGKVGLVPREAVDHWDGYHGEEVMLWDDYGMTKILDDCNKLQAIADSAPLTLNCDRIENKGMQFVSDAIVITTNAPGPAPVDFVNLGPVCRRVDFLVYCSAPEVEQIRRVSPGD

SEQ ID NO: 231 - protein, 167 aa, synthetic construct
KELLDETHRCKGASSTRVDPFHVSLYGSPGVGKSFVMGKLLDDVLDFMSEPQADRCYSKTPNEEYWSGYIGQTAVKCDDLGQDLSKGFSPTYNQIIQMKTNNCFIVPMADLANKGRTFTSKYIFSTTNVPGCGTKHGLADPGAFMRRRNIFVEVETEGDMIPGSTNH

SEQ ID NO: 232 - protein, 167 aa, synthetic construct
LELMNESYTYKGMKQSRIDPLHVCMLGAPGVGKSTIAHVVINHLLDHRGEPEVDRIYTRSCADAYWSNYHQEPVILYDDLGAIKSNLRLSDYAEIMGIKTNDPFSVPMAAVEDKGKHCTSKYVFSCTNVLNLDDTGDVVTKMAYYRRRNVLVKVERDPDVPKNEANP

SEQ ID NO: 233 - protein, 167 aa, synthetic construct
LELMNESYTYKGMKQSRIDPLHVCMLGAPGVGKSTIAHVVINNLLDHRGEPEVDRIYTRCCADAYWSNYHQEPVILYDDLGAIKSNLRLSDYAEIMGIKTNDPFSVPMAAVEDKGKHCTSKYVFSCTNVLNLDDTGDVVTKMAYYRRRNVLVKVERDPDVPKNEANP

SEQ ID NO: 234 - protein, 165 aa, synthetic construct
NYTLAISQHRKCQTGPRPEPVVVYLYGPPGTGKSLLASLLAQTLSQRLAGTPDDVYSPSSASCEYFDGYTGQTVHFIDDIGQDPEGRDWANFPNLVSSAPFIVPMASLEEKGTHYTSKVIVVTSNFHEPNERAARSMGALRRRVHLRINVTSNGVPFDPTNALNP

SEQ ID NO: 235 - protein, 165 aa, synthetic construct
YSTALSAISLVGTPGTRPEPLVVYLYGPPGTGKSLLASLLASTLAQALSGDPNNYYSPSSPDCKFYDGYSGQPVHYIDDIGQDPDGADWADFVNIVSSAPFIVPMADVNDKGRFYTSRVVIVTSNFPGPNPRSARCVAALERRLHIRLNVTARDGAAFSAAAALK

SEQ ID NO: 236 - protein, 165 aa, synthetic construct
YSTALSAISLVGTPGTRPEPLVVYLYGPPGTGKSLLASLLASTLAQALSGDPNNYYSPSSPDCKFYDGYSGQPVHYIDDIGQDPDGADWADFVNIVSSAPFIVPMADVNDKGRFYTSRVVIVTSNFPGPNPRSARCVAALERRLHIRLNVTARDGVAFSAAAALQ

SEQ ID NO: 237 - protein, 159 aa, synthetic construct
LARIPNRISTNNDLIRIEPIGIWIQGEPGQGKSFLTHTLSRQLQKSCKLNGVFTNPTASEFMDGYDNQDIHLIDDLGQTRKEKDIEMLCNCISSVPFIVPMAHLEEKGKFYTSKLVVATTNKSDFSSTVLQDSGALKRRFPYIMHIRAAKAYSKAGKLN

SEQ ID NO: 238 - protein, 162 aa, synthetic construct
FQELARIPNRISTQNDLIRVEPIGVWIQGEPGQGKSFLTHTLSRQLQKSCKLNGVYTNPTASEFMDGYDNQDIHLIDDLGQTRKEKDIEMLCNCISSVPFIVPMAHLEEKGKFYTSKLVIATTNKSDFSSTVLQDSGALKRRFPYIMHIRAAKAYSKSGKLN

SEQ ID NO: 239 - protein, 162 aa, synthetic construct
FQELARIPNRMATQNDLIRVEPIGIWIQGEPGQGKSFLTHTLSKQLQKTCGLQGIYTNPTASEFMDGYDNQDIHLIDDLGQTRKERDIEMLCNCISSDPDIVPMAHLEEKGKFYTSKLVIATTNKPDFSSTVLLDSGALRRRFPYIMHIRAAKHYSKSGKLN

SEQ ID NO: 240 - protein, 162 aa, synthetic construct
YSEMMRVNVRVPANTDLTRLEPIGIWVSSEPGQGKSFFTHMLSTCLLKSCNLEGIYTNPTGSEFMDGYIGQDIHIIDDAGQNREEKDLALLCQCISSVPFTVPMADLTEKGTFYTSKIVIATTNKFDFTSMVLTDPAALERRFPFHLRIRAVASYSRNNKLD

SEQ ID NO: 241 - protein, 173 aa, synthetic construct
LEVLLIEVKVLENSNQTRVKPVGVTIQGEKQIGKTNLVAILSRKICEYVQEHGDISFRNATKWTTWSRQCRDEFDTGYTGQEITYVDDAFQQKDNKDHLMWFTFISNTAVGTNQADLKQKGLPYRSKLVFTTCNKSPDKSVTIEDIEALHARFPHTICLRRNKNKMPKRGAID

SEQ ID NO: 242 - protein, 163 aa, synthetic construct
HQKLKNLGSINQAMVTRCEPVVCYLYGKRGGGKSLTSIALATKICKHYGVEPEKNIYTKPVASDYWDGYSGQLVCIIDDIGQNTTDEDWSDFCQLVSGCPMRLNMASLEEKGRHFSSPFIIATSNWSNPSPKTVYVKEAIDRRLHFKVEVKPASFFKNPHNDM

SEQ ID NO: 243 - protein, 163 aa, synthetic construct
HQKLKNLGSINQAMVTRCEPVVCYLYGKRGGGKSLTSIALATKICKHYGVEPEKNIYTKPVASDYWDGYSGQLVCIIDDIGQNTTDEDWSDFCQLVSGCPLRLNMASLEEKGRHFSSPFIIATSNWSNPSPKTVYVKEAIDRRLHFKVEVNPASFSKNPHNDM

SEQ ID NO: 244 - protein, 163 aa, synthetic construct
HQKLKNLGSINQAMVTRCEPVVCYLYGKRGGGKSLTSIALATKICKHYGVEPEKNIYTKPVASDYWDGYSGQLVCIIDDIGQNTTDEDWSDFCQLVSGCPMRLNMASLEEKGRHFSSPFIIATSNWSNPSPKTVYVKEAIDRRLHYKIEVKPASFYKNAHNDM

SEQ ID NO: 245 - protein, 163 aa, synthetic construct
LNKLGRLDKPNAPILVRAEPTVLYLYGNRGGGKSLASMAIAVKLCKELGISHVEGIYTKPIMSDFWDGYAGQPVVIMDDLGQSTSDEDWTNFCQLVSSCPLRLNMANLEKKGTQFNSPFIIASSNLSHPCPKTVYCTDAIARRLHIKVKVSPKEEFSTHAMLD

SEQ ID NO: 246 - protein, 163 aa, synthetic construct; identical to SEQ ID NO: 245

SEQ ID NO: 247 - protein, 163 aa, synthetic construct
VQIATYFRNFTTADSDTSRPEPVVVVLRGKPGVGKSAAATVMAAAVSKLLVGSQSVYTLSPDTEHMDGYHGQFVTLMDDLGQNPDGEDFRCFCQMVSCAQYRPAMADLKDKGILFTSRLLIATTNLPDFNPVTISDPRALDRRITFDILVTPGSAATKNGKLD

SEQ ID NO: 248 - protein, 169 aa, synthetic construct
LGKTNLAQSLTTMINAKQSSAQRVEPVVVVLRGKPGCGKSLASTLIAQAVSKRLYGSQSVYSLPPDPDFFDGYKGQFVTLMDDLGQNPDGQDFSTFCQMVSTAQFLPNMADLAEKGRPFTSNLIIATTNLPHFSPVTIADPSAVSRRINYDLTLEVSEAYKKHTRLNFD

SEQ ID NO: 249 - protein, 167 aa, synthetic construct
EKRTGIAAVCEKFRQKHDHATARCEPVVIVLRGDAGQGKSLSSQVIAQAVSKTIFGRQSVYSLPPDSDFFDGYENQFAAIMDDLGQNPDGSDFTTFCQMVSTTNFLPNMASLERKGTPFTSQLVVATTNLPEFRPVTIAHYPAVERRITFDYSVSAGPVCSKTEAGY

SEQ ID NO: 250 - protein, 167 aa, synthetic construct
EKRTGIAAVCEKFRQKHDHATARCEPVVIVLRGDAGQGKSLSSQIIAQAVSKTIFGRQSVYSLPPDSDFFDGYENQFAAIMDDLGQNPDGSDFTTFCQMVSTTNLLPNMASLERKGTPFTSQLVVATTNLPEFRPVTIAHYPAVERRITFDYSVSAGPVCSKTEAGC

SEQ ID NO: 251 - protein, 166 aa, synthetic construct
IPLATLCEKFKNKHDHAVARPEPVVVVLRGNAGQGKSVTSQIIAQAVSKLSFGRQSVYSLPPDSDYLDGYENQYSVIMDDLGQNPDGEDFKVFCQMVSSTNFLPNMAHLEKKGTPFTSNFIIATTNLPKFRPVTVAHYPAVDRRITFDLTVEAGDECVTHNGMLDV

SEQ ID NO: 252 - protein, 166 aa, synthetic construct
IPLASLCEKFKNRHDHSVTRPEPVVVVLRGAAGQGKSVTSQIIAQSVSKMAFGRQSVYSMPPDSEYFDGYENQFSVIMDDLGQNPDGEDFTVFCQMVSSTNFLPNMAHLERKGTPFTSSFIVATTNLPKFRPVTVAHYPAVDRRITFDFTVTAGPHCKTPAGMLDV

SEQ ID NO: 253 - protein, 166 aa, synthetic construct
IPLASLCEKFKNRHDHSVTRPEPVVAVLRGAAGQGKSVTSQIIAQSVSKMAFGRQSVYSMPPDSEYFDGYENQFSVIMDDLGQNPDGEDFTVFCQMVSSTNFLPNMAHLERKGTPFTSSFIVATTNLPKFRPVTVAHYPAVDRRITFDFTVTAGPHCKTPAGMLDI

SEQ ID NO: 254 - protein, 165 aa, synthetic construct
NVHIANLCKVVAPAPSKSRPEPVVVCLRGKSGQGKSFLANVLAQAISTHLTGRTDSVWYCPPDPDHFDGYNQQTVVVMDDLGQNPDGKDFKYFAQMVSTTGFIPPMASLEDKGKPFSSKVIIATTNLYSGFTPKTMVCPDALNRRFHFDIDVSAKDGYKINNKLD

SEQ ID NO: 255 - protein, 165 aa, synthetic construct; identical to SEQ ID NO: 254

SEQ ID NO: 256 - protein, 165 aa, synthetic construct
NVHIANLCKVVTPAPSKSRPEPVVVCLRGKSGQGKSFLANVLAQAISTHFTGRIDSVWYCPPDPDHFDGYNQQTVVVMDDLGQNPDGKDFKYFAQMVSTTGFIPPMASLEDKGKPFNSKVIITTTNLYSGFTPRTMVCPDALNRRFHFDIDVSAKDGYKVNNKLD

SEQ ID NO: 257 - protein, 163 aa, synthetic construct
EAKRIKVLYNKCSNIIKQRKRSEPVAVMIHGPPGTGKSITTNFLARMITNESDVYSLPPDPKYFDGYDNQSVVIMDDIMQNPDGEDMTLFCQMVSSVTFIPPMADLPDKGKPFDSRFILCSTNHSLLAPPTISSLPAMNRRFFFDLDIVVHDNYKDTQGKLDV

SEQ ID NO: 258 - protein, 161 aa, synthetic construct
KRIKVLYHKCTNIIKQKKRSEPVAVMIHGPPGTGKSITTSFLARMITNESDIYSLPPDPKYFDGYDNQSVVIMDDIMQNPGGEDMTLFCQMVSSVTFIPPMADLPDKGKPFDSRFVLCSTNHSLLAPPTISSLPAMNRRFYLDLDILVHDNYKDNQGKLDV

SEQ ID NO: 259 - protein, 163 aa, synthetic construct
EAKRIKTLYIKCDNIIKQKKRCEPVAIVIHGPPGAGKSITTNFLAKMITNDSDIYSLPPDPKYFDGYDQQSVVIMDDIMQNPAGDDMTLFCQMVSSVTFIPPMADLPDKGKAFDSRFVLCSTNHSLLTPPTITSLPAMNRRFFLDLDIIVHDNFKDPQGKLNV

SEQ ID NO: 260 - protein, 161 aa, synthetic construct
EARRIKNLYIKCSNVIKGGKRNEPVAVLIHGSPGTGKSLATSVLARMLTVETDIYSLPPDPKYFDGYDQQSVVIMDDIMQNPSGEDMTLFCQMVSSVPFIPPMADLPDKGKPFTSKFVLASTNHTLLTPPTVSSLPAMARRFYFDLDIQVKKEYLLDGKLD

SEQ ID NO: 261 - protein, 157 aa, synthetic construct
KNKMVNYMQFKSKQRIEPVCVLIHGTPGSGKSLTTSIVGRAIAEHFNSAVYSLPPDPKHFDGYQQQEVVIMDDLNQNPDGQDISMFCQMVSSVDFLPPMASLDNKGMLFTSNFVLASTNSNTLSPPTILNPEALVRRFGFDLDICLHTTYTKNGKLN

SEQ ID NO: 262 - protein, 157 aa, synthetic construct
KNKITNYMQFKSKQRTEPVCVLIHGTPGSGKSLTTSIVGRALAEHFNSSVYSLPPDPKHFDGYQQQEVVIMDDLNQNPDGQDISMFCQMVSSVDFLPPMASLDNKGMLFTSNFVLASTNSNTLSPPTILNPEALIRRFGFDLDICMHSTYTKNGKLN

SEQ ID NO: 263 - protein, 159 aa, synthetic construct
EKRVLGAMQFKTKNRIEPVCALIHGSPGTGQSLATMIVGRKLAEYEGSDVYSLPPDPDHFDGYQQQAVVVMDDLLQNPDGKDMTLFCQMVSTAPFTVPMAALEDKGKLFTSKFVLASTNAGQVTPPTVADYKALQRRFFFDCDIEVQKEYKRDGVTLDV

SEQ ID NO: 264 - protein, 159 aa, synthetic construct
EKRILGYIQFKSKQRIEPVCLLIHGTAGTGKSLATSIIGRKLAEYEHSEVYAIPPDSDHFDGYQQQAVVVMDDLNQNPDGKDMVAFCQMVSTVPYHVPMAAIEEKGMLFTSSYVLASTNSGSIHPPTVSNSKALSRRFAFDVDIEVSEHYKTHNGTLDV

SEQ ID NO: 265 - protein, 159 aa, synthetic construct
EKKINNYIQFKSKSRIEPVCLIIHGSPGTGKSVASNLIARAITEKLGGDIYSLPPDPKYFDGYKQQTVVLMDDLMQNPDGNDISMFCQMVSTVDFIPPMASLEEKGTLYTSPFLIATTNAGSIHAPTVSDSKALSRRFKFDVDIEVTDSYKDSNKLDMS

SEQ ID NO: 266 - protein, 157 aa, synthetic construct
ERKINNYIQFKSKSRIEPVCLIIHGSPGTGKSVASNLIARAITEKLGGDSYSLPPDPKYFDGYKQQTVVLMDDLMQNPDGNDIAMFCQMVSTVDFIPPMASLEEKGTLYTSPFLIATTNAGSIHAPTVSDSKALARRFKFDMEIESMESYKDGVRLD

SEQ ID NO: 267 - protein, 159 aa, synthetic construct
EKRMNNYMQFKSKHRIEPVCLIIRGSPGTGKSLATGIIARAIADKYHSSVYSLPPDPDHFDGYKQQVVTVMDDLCQNPDGKDMSLFCQMVSTVDFIPPMASLEEKGVSFTSKFVIASTNATNIIVPTVSDSDAIRRRFYMDCDIEVTDSYKTDLGRLDA

SEQ ID NO: 268 - protein, 159 aa, synthetic construct
EKRMNNYMQFKSKHRIEPVCLIIRGSPGTGKSLATGIIARAIADKYHSSVYSLPPDPDHFDGYKQQVVTVMDDLCQNPDGKDMSLFCQMVSTVDFIPPMASLEEKGVSFTSKFVIASTNASNIIVPTVSDSDAIRRRFYMDCDIEVTDSYKTDLGRLDA

SEQ ID NO: 269 - protein, 159 aa, synthetic construct
EKRMNNYMQFKSTHRIEPVCLIIRGSPGTGKSLATGIIARAIADKYHSSVYSLPPDPDHFDGYKQQVVTVMDDLCQNPDGKDMSLFYQMVSTVDIIPPMASLEEKGVSFTSKFVIASTNASNIIVPTVSDSDAIRRRFYMDCDIEVTDSSKTDLGRLDA

SEQ ID NO: 270 - protein, 156 aa, synthetic construct
ESTINNYIQFKSKHRIEPVCMLVHGSPGTGKGIASSLIGRAIAERETTSVYSVPLAPSHFDGYKQQGYDMDDLNQNPDGMDMKLFCQMVSTVEFIPPMASLEEKGILFTSDYVLASTNSHSIAPPTVAHSDALTRRFAFDVEVYTMSEHSVKGKLN

SEQ ID NO: 271 - protein, 157 aa, synthetic construct
ENTINNYIQFKSKHRIEPVCMLIHGSPGTGKSIATSLIGRAIAEKENTSTYSLPPDPTHFDGYKQQGVVIMDDLNQNPDGNDMKLFCQMVSTVEFIPPMASLEEKGILFTSDYVLASTNSHTITPPTVSHSDALNRRFAFDMEVYTMSEHSIKGKLN

SEQ ID NO: 272 - protein, 157 aa, synthetic construct
EHTINNYIQFKSKHRIEPVCLLVHGSPGTGKSVATNLIARAIAERENTSTYSLPPDPSHFDGYKQQGVVIMDDLNQNPDGADMKLFCQMVSTVEFIPPMASLEEKGILFTSNYVLASTNSSRISPPTVAHSDALARRFAFDMDIQVMNEYSRDGKLN

SEQ ID NO: 273 - protein, 157 aa, synthetic construct
EHTINNYIQFKSKHRIEPVCLLVHGSPGTGKSVATNLIARAIAEKENTSTYSLPPDPSHFDGYKQQGVVIMDDLNQNPDGADMKLFCQMVSTVEFIPPMASLEEKGILFTSNYVLASTNSSRITPPTVAHSDALARRFAFDMDIQVMSEYSRDGKLN

SEQ ID NO: 274 - protein, 157 aa, synthetic construct
EKKMSNYIQFKSKCRIEPVCLLLHGSPGVGKSVATNLIGRSLAEKLNSSIYSLPPDPDHFDGYKQQAVVIMDDLCQNPDGKDVSLFCQMVSSVDFVPPMAALEEKGILFTSPFVLASTNAGSINAPTVSDSRALARRFHFDMNIEVISMYNQNGKIN

SEQ ID NO: 275 - protein, 157 aa, synthetic construct
EKKMSNYIQFKSKCRIEPVCLLLHGSPGAGKSVATNLIGRSLAEKLNSSVYSLPPDPDHFDGYKQQAVVIMDDLCQNPDGKDVSLFCQMVSSVDFVPPMAALEEKGILFTSPFVLASTNAGSVNAPTVSDSRALVRRFHFDMNIEVVSMYSQNGKIN

SEQ ID NO: 276 - protein, 157 aa, synthetic construct
EKKMSNYIQFKSKCRIEPVCLLLHGSPGAGKSVATNLIGRSLAEKLNSSVYSLPPDPDHFDGYKQQAVVIMDDLCQNPDGGDISLFCQMVSSVDFVPPMAALEEKGILFTSPFVLASTNAGSINAPTVSDSRALARRFHFDMNIEVISMYSQNGKIN

SEQ ID NO: 277 - protein, 157 aa, synthetic construct
EKKMSNYIQFKSKCRIEPVCLLLHGSPGAGKSVATSLIGRSLAEKLNSSVYSLPPDPDHFDGYKQQAVVIMDDLCQNPDGKDVSLFCQMVSSVDFVPPMAALEEKGILFTSPFVLASTNAGSINAPTVSDSRALARRFHFDMNIEVISMYSQNGKIN

SEQ ID NO: 278 - protein, 157 aa, synthetic construct
EKKMSNYIQFKSKCRIEPVCLLLHGSPGAGKSVATNLIGRSLAEKLNSSVYTLPPDPDHFDGYKQQAVVIVDDLCQNPDGKDVSLFCQMVSSVDFVPPMAALEEKGILFTSLFVLASTNAGSINAPTVSDSRALARRFHFDMNIEVISMYSQNGKIN

SEQ ID NO: 279 - protein, 157 aa, synthetic construct
EKKMSNYIQFKSKCRIEPVCLLLHGSPGAGKSVATNLIGRSLAEKLNSSVYSLPPDPDHFDGYKQQAVVIMDDLCQNPDGKDVSLFCQMVSSVDFVPPMAALEEKGILFTSPFVLASTNAGSINAPTVSDSRALARRFHFDMNIEVISMYSQNGKIN

SEQ ID NO: 280 - protein, 157 aa, synthetic construct
EKKMSNYIQFKSKCRIEPVCLLLHGSPGAGKSVATNLIGRSLAEKLNSSVYSLPPDPDHFDGYKQQAVVIMDDLCQKPDGKDVSLFCQMVSSVDFVPPMAALEEKGILFTSPFVLASTNAGSINAPTVSDSRALARRFHFDMNIEVISMYSQNGKIN

SEQ ID NO: 281 - protein, 157 aa, synthetic construct; identical to SEQ ID NO: 279

SEQ ID NO: 282 - protein, 157 aa, synthetic construct; identical to SEQ ID NO: 279

SEQ ID NO: 283 - protein, 157 aa, synthetic construct; identical to SEQ ID NO: 279

SEQ ID NO: 284 - protein, 536 aa, synthetic construct
MPGFYEIVIKVPSDLDEHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRLARGHSL

SEQ ID NO: 285 - protein, 397 aa, synthetic construct
MELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 286 - protein, 312 aa, synthetic construct
MELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRLARGHSL

SEQ ID NO: 287 - protein, 156 aa, synthetic construct
DPQYAASVFLGWATKKFGKRNTIWLFGPATTGKTNIVEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFFFELTRRLDHDFGKVTK

SEQ ID NO: 288 - protein, 621 aa, synthetic construct
MPGFYEIVIKVPSDLDEHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIVEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFFFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 289 - protein, 621 aa, synthetic construct
MPGFYEIVIKVPSDLDEHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNFLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIVEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFFFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 290 - protein, 156 aa, synthetic construct
DPAYAGSVFLGWAQKKFGKRNTIWLFGPATTGKTNIVEAIAHAVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFFFELTRRLEHDFGKVTK

SEQ ID NO: 291 - protein, 625 aa, synthetic construct
MPGFYEIVIKVPSDLDEHLPGISDSFVNWVAEKEWELPPDSDMDRNLIEQAPLTVAEKLQRDFLVQWRRVSKAPEALFFVQFEKGESYFHLHVLVETTGVKSMVLGRFLSQIREKLGPDHLPAGSSPTLPNWFAVTKDAVMAPAGGNKVVDECYIPNYLLPKTQPELQWAWTNMEEYISACLNLAERKRLVAQHLTHVSQTQEQNKENLNPNSDAPVIRSKTSARYMELVGWLVDRGITSEKQWIQEDQASYISFNAASNSRSQIKAALDNAGKIMALTKSAPDYLVGPSLPADITQNRIYRILALNGYDPAYAGSVFLGWAQKKFGKRNTIWLFGPATTGKTNIVEAIAHAVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFFFELTRRLEHDFGKVTKQEVKEFFRWASDHVTEVAHEFYVRKGGASKRPAPDDADKSEPKRACPSVADPSTSDAEGAPVDFADRYQNKCSRHAGMLQMLFPCKTCERMNQNFNICFTHGVRDCSECFPGVSESQPVVRKRTYRKLCAIHHLLGRAPEIACSACDLVNVDLDDCVSEQ

SEQ ID NO: 292 - DNA, 1053 bp, synthetic construct
  source 1..1053: mol_type = other DNA
atgcctaaaaagaaaaggaaggtgagtaatttgcttacagtgcatcagaaccttccagcattgccagtagatgccacctccgatgaggtaaggaaaaacctcatggacatgttccgggatcggcaagcattttcagaacatacctggaaaatgcttctttcagtttgccggagttgggcagcatggtgtaaactcaataataggaagtggtttcctgctgagcccgaggacgtgcgagactatctcctttatctgcaggcaagggggcttgcagttaaaactatacaacaacacctcggccagctcaatatgcttcaccggcgcagtgggctgccccgcccaagcgattctaacgcagtgtcactcgttatgcgcaggattcggaaggagaacgtggatgcaggtgagcgcgctaaacaagcactggctttcgaaaggactgactttgaccaggtcaggtccctgatggaaaactcagacagatgccaagacatcagaaatcttgcctttcttggcattgcctacaatacattgcttcggattgccgaaatagcacgcataagggttaaagacattagtaggacagatgggggacgcatgctcatccatataggaaggactaagaccctcgtcagcaccgctggcgttgagaaagccttgtccttgggcgtcacaaagctcgtcgaacggtggatcagtgtcagcggagtcgccgacgatccaaataactacctcttctgcagggtgcgaaaaaatggcgtcgccgcaccaagtgctacttcacaattgagtaccagagctcttgaggggattttcgaggctactcatcggcttatatacggagccaaggatgatagcggtcagagatacttggcatggtctggtcacagcgctagagtgggtgccgcacgggacatggcacgcgctggagtgagcatacctgaaattatgcaagcaggcggctggactaatgtgaatattgttatgaattatatccgcaatctcgactccgaaaccggagccatggtacgcctgctcgaggacggtgactaa

SEQ ID NO: 293 - DNA, 1053 bp, synthetic construct
  source 1..1053: mol_type = other DNA
atgcccaaaaagaagaggaaagtaagtaatttgcttaccgtccatcagaatctccccgcacttcccgttgatgctacttccgatgaggttagaaagaacctgatggatatgttccgggaccgccaggctttttccgagcatacttggaagatgttgctttccgtgtgccggagctgggcagcgtggtgtaagcttaacaatagaaaatggtttcctgcggaacccgaggatgtgagagattatcttctttacttgcaggcacgaggcctcgccgtgaagacgattcagcaacacttgggccaacttaatatgctccatcgcagatccgggttgcctcgaccgagtgactctaatgcagtctcacttgtgatgcgacgaatccgaaaggaaaatgttgacgccggtgaacgagccaaacaagcactcgcattcgaacgcaccgacttcgaccaagtcaggagcctcatggaaaattcagaccgctgtcaagatatccgaaaccttgcctttctgggcatagcgtataacacattgttgaggatagcagaaatagcgaggattagggttaaggacatctctaggaccgatggaggtaggatgttgatccacatcggccgcacaaaaacactcgttagcacggcaggggtagaaaaggcactttctcttggagtaacgaaacttgttgaacgatggatttctgtgtctggagtcgcagatgacccgaataattacctcttctgccgagttaggaaaaacggcgtcgccgcgccgtctgctacaagtcaactttctacaagagcacttgagggcattttcgaagcaacgcatcgactgatttatggagcgaaggacgattctggccagcgatatctcgcgtggtccggccacagtgctcgcgtcggggcagcgcgggatatggcgcgcgcaggagtaagtataccggaaatcatgcaagcaggtggctggacgaacgttaatatcgtgatgaactatattaggaacctggatagtgaaactggagcgatggtccgcttgttggaagacggagactag

SEQ ID NO: 294 - DNA, 1053 bp, synthetic construct
  source 1..1053: mol_type = other DNA
atgcctaagaagaagaggaaggtgagcaacctgctgaccgtgcatcagaacctgcctgccctgcctgtggacgccacaagcgacgaggttaggaagaatttaatggacatgtttagggataggcaagccttcagcgagcacacctggaagatgctgctgagcgtgtgtaggagctgggccgcctggtgcaagctgaacaataggaagtggttccctgccgagcctgaggacgtgagggactacctgctgtacctgcaagctaggggcctggccgtgaagaccattcagcagcacctgggacagctgaacatgctgcataggaggagcggcctgcctaggcctagcgacagcaacgccgtgagcctggtgatgaggaggattaggaaggagaacgtggacgccggcgagagggccaagcaagccctggccttcgagaggaccgacttcgaccaagtgaggagcctgatggagaacagcgataggtgccaagacattaggaacctggctttcctgggcatcgcctacaacaccctgctgaggatcgccgagatcgctaggattagggtgaaggacatctctaggaccgacggcggaaggatgctgatccacatcggaaggaccaagaccctggtgagcaccgccggcgtggagaaggccctgagcctgggcgtgaccaagctggtggagaggtggatcagcgtgagcggcgtggccgacgaccctaacaactacctgttctgtagggtgaggaaaaatggggtagccgcccctagcgccacatctcagctgagcacaagggccctggagggcatcttcgaggccacccataggctgatctatggcgcgaaagacgacagcggacagcgctacctggcctggagcggtcacagcgctagggttggtgcggctagggacatggctagggccggcgtgagcatccctgagatcatgcaagccggcggctggaccaacgtgaacatcgtgatgaactacattaggaacctggacagtgagaccggcgccatggtgaggctgctggaggacggcgactga

SEQ ID NO: 295 - DNA, 1053 bp, synthetic construct
  source 1..1053: mol_type = other DNA
atgcccaagaagaagagaaaggtgagcaacctgctgaccgtgcatcagaacctgcccgccctgcccgtggacgccacaagcgacgaggtaagaaaaaacctcatggacatgttcagagacagacaagccttcagcgagcacacctggaagatgctgctgagcgtgtgcagaagctgggccgcctggtgcaagctgaacaacagaaagtggttccccgccgagcccgaggacgtgagagactacctgctgtacctgcaagctagaggcctggccgtgaagaccattcagcagcacctggggcagctgaacatgctgcacagaagaagcggcctgcctagacctagcgacagcaacgccgtgagcctggtgatgagaagaatcagaaaggagaacgtggacgccggcgagagagccaagcaagccctggccttcgagagaaccgacttcgaccaagtgagaagcctgatggagaacagcgacagatgccaagacataagaaacctggcctttctgggcatcgcctacaacaccctgctgagaatcgccgagatcgctagaatcagagtgaaggacatcagcagaaccgacggcggcagaatgctgatccacatcggcagaaccaagaccctggtgagcaccgccggcgtggagaaggccctgagcctgggcgtgaccaagctggtggagagatggatcagcgtgagcggcgtggccgacgaccccaacaactacctgttctgcagagtgagaaaaaatggagtggccgcccctagcgccacatctcagctgagcacaagagccctggagggcatcttcgaggccacccacagactgatctacggcgccaaggacgacagcgggcagcggtaccttgcttggagcggccacagtgcgagagtgggcgcagcccgtgacatggctagagccggcgtgagcatccccgagatcatgcaagccggcggctggaccaacgtgaacatcgtgatgaactacatcagaaatctggactctgagaccggcgccatggtgagactgctggaggacggcgactga

SEQ ID NO: 296 - DNA, 20 bp, synthetic construct
  source 1..20: mol_type = other DNA
cgaagctggtggaaagatgg

SEQ ID NO: 297 - DNA, 19 bp, synthetic construct
  source 1..19: mol_type = other DNA
caattggctggtggcagag

SEQ ID NO: 298 - DNA, 20 bp, synthetic construct
  source 1..20: mol_type = other DNA
ccgtgaagaccattcagcag

SEQ ID NO: 299 - DNA, 20 bp, synthetic construct
  source 1..20: mol_type = other DNA
cacttggtcgaagtcggttc

SEQ ID NO: 300 - DNA, 30 bp, synthetic construct
  source 1..30: mol_type = other DNA
tatgtcgtggagtctactggtgtcttcacc

SEQ ID NO: 301 - DNA, 28 bp, synthetic construct
  source 1..28: mol_type = other DNA
gttgtcatatttctcgtggttcacaccc

SEQ ID NO: 302 - DNA, 21 bp, synthetic construct
  source 1..21: mol_type = other DNA
actggtctaggacccgagaag

SEQ ID NO: 303 - DNA, 21 bp, synthetic construct
  source 1..21: mol_type = other DNA
tcaatggtgcctctggagatt

SEQ ID NO: 304 - DNA, 20 bp, synthetic construct
  source 1..20: mol_type = other DNA
agcggaccaacaagagagaa

SEQ ID NO: 305 - DNA, 20 bp, synthetic construct
  source 1..20: mol_type = other DNA
atcatcttgcagccatgcag

SEQ ID NO: 306 - DNA, 22 bp, synthetic construct
  source 1..22: mol_type = other DNA
tagaagggatatacaaagtgga

SEQ ID NO: 307 - DNA, 22 bp, synthetic construct
  source 1..22: mol_type = other DNA
tagaagggatatagaaagtatc

SEQ ID NO: 308 - DNA, 22 bp, synthetic construct
  source 1..22: mol_type = other DNA
tagcagggatatacaaagggaa

SEQ ID NO: 309 - DNA, 21 bp, synthetic construct
  source 1..21: mol_type = other DNA
ctccagcctgggtgacaagag

SEQ ID NO: 310 - DNA, 31 bp, synthetic construct
  source 1..31: mol_type = other DNA
atgtaagttcaagtcatgttactaagtggat

SEQ ID NO: 311 - DNA, 34 bp, synthetic construct
  source 1..34: mol_type = other DNA
ccattctttgtctgatttgcaatgctgatgttac

SEQ ID NO: 312 - DNA, 31 bp, synthetic construct
  source 1..31: mol_type = other DNA
gttttcactgatatgggagaatggtctgccc

SEQ ID NO: 313 - DNA, 29 bp, synthetic construct
  source 1..29: mol_type = other DNA
ggtgattggcaaactgttaggaatgaggc

SEQ ID NO: 314 - DNA, 31 bp, synthetic construct
  source 1..31: mol_type = other DNA
gtcatttctcctctttgggagttatttcctc

SEQ ID NO: 315 - DNA, 27 bp, synthetic construct
  source 1..27: mol_type = other DNA
ataggaaaaccagtgagtctggagagc

SEQ ID NO: 316 - DNA, 25 bp, synthetic construct
  source 1..25: mol_type = other DNA
ccaaaaccaaaacaaccctcgaagg

SEQ ID NO: 317 - DNA, 36 bp, synthetic construct
  source 1..36: mol_type = other DNA
accgtagtcaaacttctctgaatatttgtactagag

SEQ ID NO: 318 - DNA, 36 bp, synthetic construct
  source 1..36: mol_type = other DNA
aacctaacacaatgagaaacagaagatattacctac

SEQ ID NO: 319 - DNA, 29 bp, synthetic construct
  source 1..29: mol_type = other DNA
gtatacatgttcctgtgccttatactccc

SEQ ID NO: 320 - DNA, 24 bp, synthetic construct
  source 1..24: mol_type = other DNA
acatggcaggaaacaactcctgct

(19) United States
(12) Patent Application Publication
(10) Pub. No.: US 2026/0193673 A1
(43) Pub. Date: Jul. 9, 2026

(54) IMPROVED SYSTEM FOR PRODUCING RNA-PACKAGED AAV PARTICLES

(71) Applicant: HuidaGene Therapeutics (Singapore) Pte. Ltd., Singapore (SG)

(72) Inventors: Weiya Bai, Shanghai (CN); Zhen Liu, Shanghai (CN); Linyu Shi, Shanghai (CN)

(21) Appl. No.: 19/135,143

(22) PCT Filed: Dec. 8, 2023
(86) PCT No.: PCT/CN2023/137565

§ 371 Date: Jun. 3, 2025

(30) Foreign Application Priority Data
Dec. 8, 2022 (WO) PCT/CN2022/137625
Oct. 11, 2023 (WO) PCT/CN2023/124077

(51) Int. Cl. C12N 15/86; C07K 14/005; C12N 9/14

(52) U.S. Cl. CPC C12N 15/86; C07K 14/005; C12N 9/14; C12Y 306/04012; C12N 2750/14122; C12N 2750/14143; C12N 2750/14152

(57) ABSTRACT

Provided herein are helicase, Rep proteins comprising the helicase, and systems and methods using the Rep proteins for producing RNA-packaged AAV particles.

### REFERENCE TO RELATED APPLICATION

The instant application claims the priority to and the benefit of the filing date of PCT/CN2022/137625, filed on Dec. 8, 2022, and the filing date of PCT/CN2023/124077, filed on Oct. 11, 2023, the entire contents of which, including any drawings and sequence listing, are incorporated herein by reference.

### REFERENCE TO AN ELECTRONIC SEQUENCE LISTING

The disclosure contains a Sequence Listing XML file which has been submitted electronically in XML format and is hereby incorporated by reference in its entirety. Said XML copy, created on Dec. 8, 2023, by software “WIPO Sequence” according to WIPO Standard ST.26, is named HGP030PCT.xml, and is 526,281 bytes in size.

According to WIPO Standard ST.26, symbol “t” is used to denote both T in DNA and U in RNA. Thus, in the instant sequence listing prepared according to ST.26, wherever a sequence is an RNA, the T in the sequence shall be deemed as U.

### BACKGROUND

The applicant's previous WO2022/166954 (PCT/CN2022/075366), which, including any drawings and sequence listing thereof, is incorporated herein by reference in its entirety, presented an RNA sequence capable of being packaged into a DNA virus (e.g., AAV) viral particle to produce an RNA-packaged DNA virus viral particle (e.g., RNA-packaged AAV (RAAV) particles), and a system of packaging the RNA sequence into the DNA virus (e.g., AAV) viral particle. It would be desired to increase desired RNA packaging and/or reduce undesired DNA packaging of the (e.g., RAAV) packaging system.

Citation or identification of any document in the disclosure is not an admission that such a document is available as prior art to the disclosure. Each of the references mentioned or cited in the disclosure is incorporated by reference in its entirety.

### SUMMARY

The disclosure satisfies the above desire by providing an RAAV packaging system with increase RNA packaging ability (efficiency) and/or reduce DNA packaging ability (efficiency) by using a Rep protein containing a mutated helicase domain believed to have increased unwinding RNA property and/or have decreased DNA unwinding property.

In an aspect, the disclosure provides a Rep (e.g., Rep78, Rep68, Rep52, Rep40) protein comprising a helicase domain comprising an amino acid mutation relative to a reference helicase domain (e.g., SEQ ID NO: 186).

In another aspect, the disclosure provides a polynucleotide encoding a Rep (e.g., Rep78, Rep68, Rep52, Rep40) protein comprising a helicase domain comprising an amino acid mutation relative to a reference helicase domain (e.g., SEQ ID NO: 186).

In yet another aspect, the disclosure provides a polynucleotide encoding a Rep78 protein, a Rep68 protein, a Rep52 protein, and a Rep40 protein, wherein the Rep78 protein, the Rep68 protein, the Rep52 protein, and the Rep40 protein share a helicase domain comprising an amino acid mutation relative to a reference helicase domain (e.g., SEQ ID NO: 186).

In yet another aspect, the disclosure provides a helicase comprising a helicase domain comprising an amino acid mutation relative to a reference helicase domain (e.g., SEQ ID NO: 186).

In yet another aspect, the disclosure provides a polynucleotide encoding a helicase comprising a helicase domain comprising an amino acid mutation relative to a reference helicase domain (e.g., SEQ ID NO: 186).

In yet another aspect, the disclosure provides an RAAV packaging system, and specifically, a system for packaging an RNA into an AAV capsid to produce a recombinant RNA-packaged AAV particle (rRAAV particle), wherein the RNA comprises:
(a) an RNA sequence of interest (RSI), e.g., an RNA sequence encoding a protein of interest, and
(b) an RNA-packaging signal (RPS) capable of interacting, e.g., binding, directly or indirectly, with an RPS-interacting molecule that facilitates packaging of the RNA into the AAV capsid;
wherein the system comprises:
(1) one or more capsid proteins (e.g., VP1, VP2, and/or VP3) for assembling the AAV capsid, or one or more coding sequences therefor, or a polynucleotide comprising said coding sequences;
(2) one or more Rep proteins (e.g., Rep78, Rep68, Rep52, Rep40) comprising a helicase domain comprising an amino acid mutation relative to a reference helicase domain (e.g., SEQ ID NO: 186), or one or more coding sequences therefor, or a polynucleotide comprising said coding sequences;
(3) the RPS-interacting molecule, or a coding sequence therefor, or a polynucleotide comprising said coding sequence;
(4) the RNA, or a coding sequence therefor, or a polynucleotide comprising said coding sequence, e.g., a transgene vector comprising or encoding the RNA; and
(5) optionally, one or more helper proteins required for AAV packaging (e.g., helper proteins from adenoviral E2a, E4, and/or VA genes), or one or more coding sequences therefor, or a polynucleotide comprising said coding sequences.

In yet another aspect, the disclosure provides an RAAV packaging method, and specifically, a method for the production of a recombinant RNA-packaged AAV particle (rRAAV particle), said method comprising:
a) culturing for a sufficient time a cell comprising a system for packaging an RNA into a AAV capsid to produce the recombinant RNA-packaged AAV particle (rRAAV particle), and
b) harvesting the rRAAV particle or a population thereof;
wherein the RNA comprises:
(a) an RNA sequence of interest (RSI), e.g., an RNA sequence encoding a protein of interest, and (b) an RNA-packaging signal (RPS) capable of interacting, e.g., binding, directly or indirectly, with an RPS-interacting molecule that facilitates packaging of the RNA into the AAV capsid;
wherein the system comprises:
(1) one or more capsid proteins (e.g., VP1, VP2, and/or VP3) for assembling the AAV capsid, or one or more coding sequences therefor, or a polynucleotide comprising said coding sequences;
(2) one or more Rep proteins (e.g., Rep78, Rep68, Rep52, Rep40) comprising a helicase domain comprising an amino acid mutation relative to a reference helicase domain (e.g., SEQ ID NO: 186), or one or more coding sequences therefor, or a polynucleotide comprising said coding sequences;
(3) the RPS-interacting molecule, or a coding sequence therefor, or a polynucleotide comprising said coding sequence;
(4) the RNA, or a coding sequence therefor, or a polynucleotide comprising said coding sequence, e.g., a transgene vector comprising or encoding the RNA; and
(5) optionally, one or more helper proteins required for AAV packaging (e.g., helper proteins from adenoviral E2a, E4, and/or VA genes), or one or more coding sequences therefor, or a polynucleotide comprising said coding sequences.

In yet another aspect, the disclosure provides use of a Rep protein (e.g., Rep78, Rep68, Rep52, Rep40) comprising a helicase domain comprising an amino acid mutation relative to a reference helicase domain (e.g., SEQ ID NO: 186), or a polynucleotide encoding the Rep protein, in the production of a recombinant RNA-packaged AAV particle (rRAAV particle), said production comprising:
a) culturing for a sufficient time a cell comprising a system for packaging an RNA into a AAV capsid to produce the recombinant RNA-packaged AAV particle (rRAAV particle), and
b) harvesting the rRAAV particle or a population thereof;
wherein the RNA comprises:
(a) an RNA sequence of interest (RSI), e.g., an RNA sequence encoding a protein of interest, and
(b) an RNA-packaging signal (RPS) capable of interacting, e.g., binding, directly or indirectly, with an RPS-interacting molecule that facilitates packaging of the RNA into the AAV capsid;
wherein the system comprises:
(1) one or more capsid proteins (e.g., VP1, VP2, and/or VP3) for assembling the AAV capsid, or one or more coding sequences therefor, or a polynucleotide comprising said coding sequences;
(2) one or more said Rep proteins (e.g., Rep78, Rep68, Rep52, Rep40), or one or more coding sequences therefor, or a polynucleotide comprising said coding sequences;
(3) the RPS-interacting molecule, or a coding sequence therefor, or a polynucleotide comprising said coding sequence;
(4) the RNA, or a coding sequence therefor, or a polynucleotide comprising said coding sequence, e.g., a transgene vector comprising or encoding the RNA; and
(5) optionally, one or more helper proteins required for AAV packaging (e.g., helper proteins from adenoviral E2a, E4, and/or VA genes), or one or more coding sequences therefor, or a polynucleotide comprising said coding sequences.

In yet another aspect, the disclosure provides a vector comprising the polynucleotide of the disclosure; optionally, wherein the vector is a plasmid.

In yet another aspect, the disclosure provides a cell, an isolated cell, a host cell, or an isolated host cell that comprises the Rep protein, the helicase, the polynucleotide, the system, or the vector of the disclosure.

In yet another aspect, the disclosure provides a recombinant RNA-packaged AAV particle (rRAAV particle) or a population thereof produced by the method of the disclosure.

The details of one or more embodiments of the disclosure are set forth in the description below. Other features or advantages of the disclosure will be apparent from the following drawings and detailed description of several embodiments, and also from the appended claims. It is understood that any aspect or embodiment of the disclosure can be combined with any other one or more aspects or embodiments of the disclosure, including aspects or embodiments only described in one sub-section, only in the examples, or only in the claims, to constitute another embodiment explicitly or implicitly disclosed herein unless otherwise indicated.

### BRIEF DESCRIPTION OF THE DRAWINGS

An understanding of the features and advantages of the disclosure will be obtained by reference to the following detailed description that sets forth illustrative embodiments, in which the principles of the disclosure may be utilized, and the accompanying drawings of which:

FIG. 1A shows the structure and sequence of the wild type ITR of AAV2, including the A:A′ stem region sequences, the B:B′ and C:C′ T region sequences, and the unpaired D region sequence, in both the flip (SEQ ID NO: 5) and flop (SEQ ID NO: 28) configuration of 3′ ITR. The RBE, RBE′ and the TRS are also shown.

FIGS. 1B and 1C show multi-sequence alignments of 5′ (SEQ ID NOs: 40, 25, 27, 29, 31, 33, 35, and 37, respectively, in order of appearance) (FIG. 1B) and 3′ (SEQ ID NOs: 41, 26, 28, 30, 32, 34, 36, and 38, respectively, in order of appearance) (FIG. 1C) ITR sequences from AAV1-7.

FIG. 2 shows the life cycle of an AAV vector/viral particle, and the subject RAAV vector/viral particle.

FIG. 3 is a schematic diagram of transgene plasmids of RAAV-ITR vectors and control vectors, showing the relative position and orientation of the promoter (such as the CAG promoter or “C”), the GOI coding sequence (such as the coding sequence for the reporter gene tdTomato or “T”), the WPRE sequence (or “W”), the SV40 polyA signal sequence (or “S”), and the wild-type ITR, mutated/optimized ITR (dITR or dITR-D).

FIG. 4 is a schematic diagram showing the generation of AAV vectors and RAAV-ITR vectors with the triple-plasmid system. By co-transfecting three plasmids (e.g., a transgene plasmid, a packaging plasmid, and a helper plasmid) into a proper packaging cell like such as the HEK293 cells, recombinant AAV or RAAV viral vectors can be generated. Green ITR indicates wild type ITR, yellow ITR indicates optimized ITRs. pCAG-Transgene, pCAG-Transgene-ITR and pCAG-ITR-Transgene-ITR are transgene plasmids; pAAV-rep/cap is a packaging plasmid; and pHelper is the helper plasmid.

FIGS. 5A and 5B show representative viral vector titration process. FIG. 5A is a flowchart for RAAV titration. FIG. 5B shows primers and probes for Q-PCR.

FIG. 6A-6C show titration of RAAV-ITR vectors. FIG. 6A shows titration of CITWS group. FIG. 6B shows titration of CTWIS group. FIG. 6C shows titration of CITWIS group.

FIGS. 7A and 7B show titration and infection of RAAV-dITR-D vectors. FIG. 7A shows titration of RAAV-dITR-D vectors. FIG. 7B shows in vitro infection of RAAV-dITR-D vectors. The same volume (5 μL) of purified RAAV-dITR-D vectors had been used to infect 2×10⁵ HEK293T cells in vitro. Fluorescence photos were taken 3 and 5 days post infection.

FIG. 8A is a schematic diagram (not to scale) showing the different plasmid constructs used to demonstrate efficient packaging of RNA into RAAV particles.

FIG. 8B shows the results of specific DNA and RNA packaging of the AAV-tdTomato and RAAV-tdTomato constructs by detecting the WPRE sequence in the packaged DNA or RNA. Efficient RNA packaging occurred when both the heterologous RNA Packaging Signal (RPS) and its cognate RPS binding protein (RBP, e.g., MCP for MS2) are both present.

FIG. 9A-9C show reduced DNA packaging using enlarged plasmid backbone. FIG. 9A is a schematic diagram (not to scale) of the various plasmids, including the plasmid with the longer backbone sequence due to the inserted stuffer region (L-CTWM3S), used to generate the results in FIGS. 9B and 9C. FIG. 9B shows specific DNA packaging of AAV-tdTomato and RAAV-tdTomato by detecting the presence of CAG promoter sequence using CAG-specific primer pairs. FIG. 9C shows specific DNA and RNA packaging of AAV-tdTomato and RAAV-tdTomato by detecting the presence of WPRE sequence using WPRE-specific primer pairs. The results showed a surprising ~2-fold reduction of undesired DNA packaging by using enlarged/longer plasmid backbone sequence with stuffer sequences.

FIGS. 10A and 10B show efficient packaging of the Cre transgene into RAAV using the MS2/MCP packaging system.

FIG. 10A shows specific DNA packaging of AAV-Cre and RAAV-Cre by detecting the presence of CAG promoter sequence using CAG-specific primer pairs. Note that the CAG sequence is not present in the RAAV RNA sequence, and the detected RNA signal was background. FIG. 10B shows specific DNA and RNA packaging of AAV-Cre and RAAV-Cre by detecting the presence of WPRE sequence using WPRE-specific primer pairs.

FIG. 11A-11B show that RPS/RBP improved RNA packaging of conventional AAVs. FIG. 11A shows the results of AAV genome packaging in the presence of only DNA packaging signals (i.e., ITRs). FIG. 11B shows the AAV genome packaging in presence of both DNA packaging signals (ITRs) and RNA packaging signals (MS2X3).

FIG. 12A-12D show the results of optimizing the RAAV system and identification of the properties of optimized RAAVs. FIG. 12A represents the specific genome packaging of AAV-Cre and RAAV-Cre by detecting WPRE sequence. FIG. 12B represents the specific genome packaging of AAV-Cre and RAAV-Cre by detecting Cre sequence. FIG. 12C shows silver staining analysis of the composition of the AAV and RAAV particles. FIG. 12D shows the morphology analysis of the AAV and RAAV particles by TEM, scale bar 100 nm.

FIGS. 13A and 13B show results of reducing DNA packaging of AAV and RAAV. FIG. 13A shows that engineered Rep reduced DNA packaging of the conventional AAV. FIG. 13B shows reduction of DNA packaging in RAAV by using various mutant MCP fusion proteins, including double mutant MCP fusion protein DJ-MCPX2.

FIG. 14A-14D show that the RAAV viral particles express functional transgene-encoded proteins. Samples are designated the same way in FIG. 14A-14C. FIG. 14A shows a time course of Cre mRNA levels in infected cells. FIG. 14B shows fold change of Cre mRNA levels in infected cells from 20 hrs post infection. FIG. 14C shows a time course of Cre DNA levels in infected cells. FIG. 14D shows percentage of infected cells quantified by flow cytometry 5 days after infection, n=2 replicates.

FIG. 15A-15D show results of DNA and mRNA analysis for the AAV or RAAV infected Ai9-MEF cells. FIG. 15A shows Ct value of the Cre mRNA. FIG. 15B shows Ct value of the Cre DNA. FIG. 15C shows Ct value of the GAPDH mRNA. FIG. 15D shows Ct value of the 36B4 DNA.

FIG. 16 shows genotype identification of Ai9-MEF cells.

FIG. 17A-17B show transient transfer of RAAV particles. FIG. 17A shows Western blot analysis of the lifespan of Cre protein in infected cells after conventional AAV delivery. FIG. 17B shows Western blot analysis of the lifespan of Cre protein in infected cells after RAAV delivery.

FIG. 18 shows additional functional RPS/RBP pairs—the PP7/PCP pair, and the com/COM pair—tested in the RAAV system.

FIG. 19 shows that the RAAV system is applicable for various AAV serotypes, including AAV-DJ, AAV5, AAV8, and AAV9.

FIGS. 20A and 20B shows that additional AAP and MCP fusion proteins increased RAAV yield. FIG. 20A represents the specific genome packaging of RAAV-Cre by detecting Cre sequence. FIG. 20B shows comparison of the RNA packaging efficiency of RAAVs with AAP N- or C-terminal fusions (AM or MA fusion constructs).

FIG. 21A-21D show results of transient transfer of RAAV-Cre into the hippocampus of Ai9-Mice. FIG. 21A shows transfer of high dose of AAV-Cre into the hippocampus of Ai9-Mice. FIG. 21B shows transfer of low dose of AAV-Cre into the hippocampus of Ai9-Mice. FIG. 21C shows transfer of high dose of RAAV-Cre into the hippocampus of Ai9-Mice. FIG. 21D shows the results in a control mouse. Red signal: tdTomato; Green signal: Cre; Blue signal: DAPI (nuclei staining).

FIG. 22 shows establishment of RAAV systems for producing mRNA-carrying AAVs. FIG. 22A. The principle of conventional AAV production. ITRs, inverted terminal repeats; CAG, CAG promoter; WPRE, Woodchuck Hepatitis Virus Posttranscriptional Regulatory Element; pA, poly(A). FIG. 22B. Schematic of the establishment of the RAAV system. RPS, RNA packaging signal; RBP, RPS binding protein. The RBP is fused to the N terminus of Rep78/68. FIG. 22C and FIG. 22D. Quantification of the tdTomato coding RNA and DNA encapsidated in RAAVs and AAVs by RT-qPCR and qPCR using primers targeting WPRE (FIG. 22C) and CAG promoter (FIG. 22D). RAAV-v1, the first generation of RAAV system; MCP, MS2 coat protein; 1× or 3×MS2, one copy or three copies of MS2 stem-loops; vg per dish, vector genomes per 15-cm dish. Data are shown as individual data points and mean values±SD for n=3 biological replicates. FIG. 22E. Quantification of the Cre coding RNA and DNA encapsidated in RAAVs and AAVs by RT-qPCR and qPCR using primers targeting WPRE. Data are shown as individual data points and mean values±range for n=2 biological replicates. FIG. 22F. The principle of the RAAV-v2 system. The Y156F mutation was introduced into the MCP-fused Rep78/68 in RAAV-v1 to abolish their endonuclease activities, thus hindering undesired DNA release and packaging. FIG. 22G. Quantification of the Cre RNA and DNA encapsidated in RAAVs generated by RAAV-v2 system by RT-qPCR and qPCR using primers targeting WPRE. Data are shown as individual data points and mean values±SD for n=3 biological replicates; unpaired, two-tailed t-test; **P<0.01; ns, not significant. FIG. 22H. Two other RPS/RBP pairs were tested in the RAAV-v2 system. PP7, PP7 binding site; PCP, PP7 bacteriophage coat protein; com, Com binding site; COM, phage COM protein. FIG. 22I. Different AAV serotypes were tested in the RAAV-v2 system.

FIG. 23 shows helicase engineering to improve RAAV productivity. FIG. 23A. Schematic of the helicase domain in AAV2 Rep78 protein. All AAV2 Rep proteins (including Rep78, Rep68, Rep50, and Rep42) contain the helicase domain. FIG. 23B. Workflow for the helicase mutagenesis experiment. An artificial capsid DJ was used to produce RAAVs. ssDNA, single-stranded DNA; ssRNA, single-stranded RNA. FIG. 23C. Measurement of single helicase mutations in RAAV production. The dashed line was set at Y=3. Top candidates are labeled as red dots. FIG. 23D. Measurement of combined helicase mutations in RAAV production. Combine single mutations originating from the same or different motifs and evaluate their RNA packaging ability. The dashed line was set at Y=4. Top candidates are labeled as red dots. FIG. 23E. Compare the RNA and DNA packaging capability of mutated helicases to the wild-type helicase in the RAAV-v2 system. Data are shown as individual data points and mean values±range for n=2 biological replicates. FIG. 23F. Quantification of the Cre RNA and Cre DNA encapsidated in RAAVs generated by RAAV-v3 system by RT-qPCR and qPCR using primers targeting WPRE. optCre, optimized Cre coding sequence 4. vg per dish, vector genomes per 15-cm dish. Data are shown as individual data points and mean values±SD for n=3 biological replicates; unpaired, two-tailed t-test, *P<0.05, **P<0.01; ns, not significant.

FIG. 24 shows characterization of the properties of RAAVs. FIG. 24A. Morphology analysis of RAAV and AAV by transmission electron microscopy. An artificial capsid DJ was used to produce RAAV and AAV. The bottom images are magnified versions of the image. Scale bars, 50 nm. FIG. 24B. Analyzing the composition of AAV and RAAV by silver staining. FIG. 24C and FIG. 24D. Characterization of the purity of AAV-DJ-Cre (C) and RAAV-DJ-v3-optCre (D) by analytical ultracentrifugation assay. FIG. 24E. Schematic of viral vector genome analysis on denaturing agarose gel. The theoretical size of the genome of RAAV-DJ-v3-optCre and AAV-DJ-Cre are displayed in the image. FIG. 24F. Analyzing the genome of RAAVs and AAVs on a denaturing agarose gel stained with SYBR™ Green IL. DNaseI and RNaseI treatment groups were set to identify the RAAV genome. FIG. 24G. Workflow for sequencing-based analysis of RAAV genome. An artificial capsid DJ was used to produce RAAVs and AAVs. FIG. 24H. Differential mRNA abundance and significance of the VLP fraction in the presence or absence of MCP. FIG. 24I. Alignment of sequencing reads showing sequencing coverage of the optCre mRNA from FIG. 24G and FIG. 24H. FIG. 24J. Schematic of transgene expression cassettes with varying lengths used to assess RAAV packaging capacity. Four qPCR primers targeting different regions of the mRNAs transcribed from the transgene cassettes are displayed. An artificial capsid DJ was used to produce RAAVs and AAVs. SA, splice acceptor site. FIG. 24K. Assess RAAV packaging capacity. Quantification of the RNA and DNA encapsidated in RAAVs by RT-qPCR and qPCR using primers targeting different regions of mRNAs. vg per dish, vector genomes per 15-cm dish. Data are shown as individual data points and mean values±range for n=2 biological replicates.

FIG. 25 shows that RAAVs enable efficient transfer of mRNA to target cells to transiently express functional proteins. FIG. 25A. Schematic of RAAV/AAV in vitro infection assay. An artificial capsid DJ was used to produce RAAVs, AAVs, and negative controls of AAVs/RAAVs (no MS2, no MCP and no Cap). Vector genome titer was used for MOI calculation. DNA amounts were normalized among RAAV, RAAV with no MS2, and RAAV with no MCP. The volume was normalized between RAAV and RAAV with no Cap for infection. For all Ai9-MEFs infection experiments, cells were plated on 48-well plates at a density of 5E4 cells per well 24 hours before infection. FIG. 25B. Investigating the infectivity of RAAVs and AAVs by analyzing the percentage of tdTomato+Ai9-MEFs 5 days after infection by cytometry. Data are shown as individual data points and mean values±SD for n=3 biological replicates, multiple unpaired t-test; ns, not significant. FIG. 25C and FIG. 25D. Time course of Cre DNA (C) and mRNA (D) levels in RAAVs/AAVs infected Ai9-MEFs. Mock: uninfected control. Data are shown as mean values±SD for n=3 biological replicates. FIG. 25E. Western blot analysis of Cre protein amount and lifespan in RAAV/AAV infected Ai9-MEFs. RAAV-DJ-v3-optCre infected Ai9-MEFs at MOI of 3,000 vg, and AAV-DJ-Cre infected Ai9-MEFs at MOI of 300 vg. Tubulin is used as a loading control. FIG. 25F. Spatial distribution of viral RNA and DNA in RAAV/AAV infected cells over time. Hela cells were infected with RAAV/AAV. At various time points after infection, the cells were fixed and processed for RNAscope analysis. Nuclei were visualized using DAPI staining. Viral RNA and DNA were detected with a DNA probe that binds to the Cre mRNA and DNA. Red arrow, aggregated RNA signal in the cytoplasm; Scale bars, 50 μm. FIG. 25G. Indels at the hTTR locus in hTTR-gRNA-HEK293T cells infected with RAAV-DJ-v3- or AAV-DJ-Cas12Max. Indels were quantified by NGS 120 hours after viral vector addition. FIG. 25H. NGS analysis of the off-target effect in hTTR-gRNA-HEK293T cells infected with RAAV-DJ-v3- or AAV-DJ-Cas12Max. The off-target site for hTTR-gRNA was predicted by Cas-OFFinder (33). Indels at the predicted off-target site were quantified by NGS 120 hours after RAAV/AAV infection. FIG. 25I. Quantification of Cas12Max DNA copy number in RAAV/AAV infected hTTR-gRNA-HEK293T cells. (G to I) Mock: uninfected control. Vector genome titer was used for MOI calculation. Data are mean values±SD with n=3 biological replicates, unpaired, two-tailed t-test; *P<0.05, ****P<0.0001; ns, not significant.

FIG. 26 shows that RAAVs enable tropism-dependent mRNA delivery to target tissues/organs to transiently express functional proteins. FIG. 26A. Experimental workflow for RAAV/AAV-DJ in vivo hippocampus transduction. DNA amounts were normalized between RAAV and RAAV with no MCP (negative control). FIG. 26B. Fluorescence microscopy analysis of the expression of tdTomato and Cre in the hippocampus of Ai9 mice 4 weeks after stereotaxic injection with AAV/RAAV. The right images are magnified versions of the area indicated by the white box in the left images. Scale bars, 200 μm in left images and 50 μm in right images. FIG. 26C. Statistic analysis of tdTomato- and Cre-positive cells in (B). Data are shown as individual data points and mean values±SD for n=3 mice; unpaired, two-tailed t-test; **P<0.01; ns, not significant. FIG. 26D. Schematic of AAV/RAAV in vivo systemic infection assay. RAAVs (carrying optCre mRNA) were generated by the RAAV-v3 system. Vector genome titer was used for the MOI calculation. FIG. 26E. Transduction of RAAVs/AAVs in the liver of Ai9 mice 4 weeks after intravenous injection. N=2 mice. Scale bars, 1000 m. FIG. 26F. Fluorescence microscopy analysis of the expression of tdTomato and Cre in the liver of Ai9 mice 4 weeks after intravenous injection with RAAV9/AAV9. N=2 mice. Scale bars, 100 μm. FIG. 26G. Statistic analysis of tdTomato- and Cre-positive cells in (F). Data are shown as individual data points and mean values±range for n=2 mice. FIG. 26H. Transduction of RAAVs/AAVs in the brain of Ai9 mice 4 weeks after intravenous injection. N=2 mice. Scale bars, 1000 μm. FIG. 26I. Fluorescence microscopy analysis of the expression of tdTomato and Cre in the brain of Ai9 mice 4 weeks after intravenous injection with RAAV/AAV-PHP.eB. N=2 mice. Scale bars, 50 μm. FIG. 26J and FIG. 26K. Statistic analysis of tdTomato- (J) and Cre- (K) positive cells in (1). Data are shown as individual data points and mean values±range for n=2 mice.

FIG. 27A shows phylogenetic analysis of the helicase domains of 98 SF3 viral helicase using AlignX program of Vector NTI software.

FIG. 27B shows alignment of the helicase domains of 98 SF3 viral helicases using AlignX program of Vector NTI software. The mutations tested are marked with black boxes.

FIG. 27C shows alignment of the helicase domains of 98 SF3 viral helicases using MUSCLE program of Jalview software. The mutations tested are marked with black boxes.

FIG. 27D shows alignment of 23 full viral-protein sequences using AlignX program of Vector NTI software. Full sequences of randomly selected 23 helicase-containing viral proteins were aligned via AlignX, and partial alignment results were displayed. The mutations tested are marked with black boxes.

FIG. 28 shows cargo sequence optimization for improving RAAV infectivity. FIG. 28A. Quantification of the encapsidation of different Cre coding sequences in RAAVs by qPCR and RT-qPCR using primers targeting WPRE. Cre opt sequences were obtained by codon optimization using several online tools (Table.S4). RAAVs were generated by the RAAV-v2(harboring the L454F+D455F mutation in helicase) system. Data are shown as individual data points and mean values±range for n=2 biological replicates. FIG. 28B. Investigating the infectivity of RAAVs carrying different Cre coding sequences by analyzing the percentage of tdTomato+Ai9-MEFs 5 days after infection by cytometry. An artificial capsid DJ was used to produce AAV/RAAV vectors. Vector genome titer was used for the MOI calculation. Data are mean values±SD with n=3 biological replicates; unpaired, two-tailed t-test; ***P<0.001, ****P<0.0001.

FIG. 29 shows analyzing RAAV genome on denaturing agarose gels stained with SYBR™ Green II. DNaseI and RNaseI treatment groups were set to identify the RAAV genome. These are full images of FIG. 3F, the lanes within the white boxes are irrelevant samples.

FIG. 30 shows RAAV packaging specificity. FIG. 30A. Differential RNA abundance of the VLP fraction in the presence or absence of MCP. FIG. 30B. Only RPS-harboring mRNA (optCre) was efficiently packaged in RAAV.

FIG. 31 shows representative flow cytometry gating scheme for AAV/RAAV in vitro Ai9-MEF infection experiments. Cells were first gated on FSC and SSC to remove debris. Following the singlets were gated on SSC. The tdTomato+ cells were gated based on uninfected controls (mock).

FIG. 32 shows titration of specific controls of AAV and RAAV. Quantification of the encapsidated DNA and RNA in the VLP fraction of AAV/RAAVs and their controls by qPCR and RT-qPCR using primers targeting WPRE.

FIG. 33 shows investigation of the effects of the transcription inhibitor actinomycin D on the viral DNA and RNA levels in AAV/RAAV-DJ-infected Ai9-MEFs. Analyzing the viral DNA (FIG. 33A) and RNA (FIG. 33B) levels in AAV/RAAV infected Ai9-MEFs. Vector genome titer was used for the MOI calculation. Ai9-MEFs were infected by AAV-Cre or RAAV-v3-optCre at MOI 1000 vg, actinomycin D was added to the cells at a concentration of 5 μg/mL 2 hours post-infection, and cells were collected at 6- and 24-hours post-infection for analyzing viral DNA and RNA. Mock: uninfected control. Data are shown as mean values±SD for n=3 biological replicates.

FIG. 34 shows that cellular DNA/mRNA were analyzed as loading controls in AAV/RAAV-DJ infected Ai9-MEFs. FIG. 34A. Ct value of cellular housekeeping gene (36B4). FIG. 34B. Ct value of cellular GAPDH mRNA. Mock: uninfected control. Data are shown as mean values±SD for n=3 biological replicates.

FIG. 35 shows investigation of the effects of the vacuolar H+-ATPase inhibitor bafilomycin A 1 and the transcription inhibitor actinomycin D on AAV/RAAV-DJ-mediated infection. FIG. 35A. Schematic of the experiment. Hela cells were treated with bafilomycin A1 (100 nM) 2 h prior to infection with AAV-DJ-Cre (MOI 1,000 vg) or RAAV-DJ-v3-Cre (MOI 10,000 vg), and the transcription inhibitor actinomycin D was added at a concentration of 5 g/mL 1 h or 6 h post-infection. DMSO was set as solvent control. At 24 hours post-infection, the cells were fixed and processed for RNAscope analysis. FIG. 35B. The effects of the vacuolar H+-ATPase inhibitor bafilomycin A1 and the transcription inhibitor actinomycin D on AAV-/RAAV-mediated transduction. Nuclei were visualized using DAPI staining. Viral RNA and DNA were detected with a DNA probe that binds to the Cre mRNA and DNA. Scale bars, 50 m.

FIG. 36 shows NGS analysis of the off-target effects in hTTR-gRNA-HEK293T cells treated with AAV-DJ- or RAAV-DJ-Cas12Max. Another off-target site for hTTR-gRNA was predicted by Cas-OFFinder (33). Indels at the predicted off-target site 2 were quantified by NGS 120 hours after infection. Data are shown as individual data points and mean values±SD with n=3 biological replicates.

FIG. 37 shows quantification of viral DNA and RNA copy number in AAV/RAAV-DJ infected hippocampus tissues. Mock: uninfected control. Data are shown as individual data points and mean values±range for n=2 biological replicates.

FIG. 38 shows that two additional AAV capsids were tested in RAAV-v3. Quantification of the encapsidated DNA and RNA in AAV/RAAVs by qPCR and RT-qPCR using primers targeting WPRE. Data are shown as individual data points and mean values±range for n=2 biological replicates.

FIG. 39 shows that RAAV with No-MCP showed no infectivity. FIG. 39A. Infection of RAAV9 with No-MCP in the liver of Ai9 mice 4 weeks after intravenous injection. N=2 mice. FIG. 39B. Transduction of RAAV-PHP.eB with No-MCP in the brain of Ai9 mice 4 weeks after intravenous injection. N=2 mice.

FIG. 40 shows investigation of the cellular tropism of RAAVs. Comparison of the infectivity of AAVs and RAAVs in Ai9-MEFs (FIG. 40A) and HEK293T Cre reporter cells (FIG. 40B). RAAVs were generated by RAAV-v2. Vector genome titer was used for the MOI calculation. Investigating the infectivity of AAV and RAAV by analyzing the percentage of tdTomato+ cells 5 days after infection by cytometry. Data are shown as individual data points and mean values±SD for n 3 biological replicates.

The figures herein are for illustrative purposes only and are not necessarily drawn to scale.

### DETAILED DESCRIPTION

### 1. Overview

Adeno-associated viruses (AAVs) are commonly used vectors for DNA delivery in gene therapy. Here the inventor developed a system that enables the AAV shell to package RNAs by introduction of RNA-packaging components and/or modification of AAV Rep proteins. The resultant RNA-carrying AAVs (RAAVs) retained properties of conventional AAVs, including capsid composition, virus morphology, and tissue tropism. These RAAVs could mediate RNA (e.g., mRNA) transfer into target cells and tissues, leading to transient expression of the functional protein. Importantly, it is demonstrated that intravenously injected RAAVs efficiently crossed the blood-brain barrier (BBB) and infected the whole mouse brain. Thus, the DNA viral vector could be modified for RNA delivery, and the RAAV of the disclosure represents the first highly efficient BBB-crossing mRNA delivery system that could be used for therapeutic purposes via whole-brain infection.

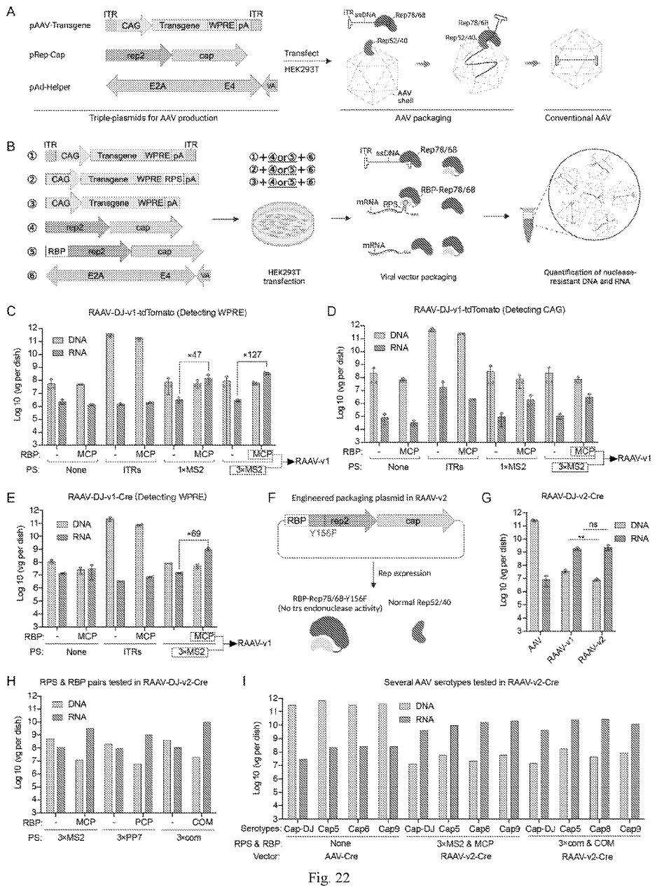

Messenger RNAs (mRNAs) have emerged as a new category of therapeutic agents for prevention and treatment of many diseases. For introducing exogenous mRNAs in vivo, the delivery system needs to protect the nucleic acid from degradation and allow effective cellular uptake and mRNA release (1). Lipid nanoparticles (LNPs) have been developed as a RNA-delivery system, and used clinically for the delivery of siRNA drugs (2) and mRNA vaccines (3-5), as exemplified by its use in delivering antigen mRNAs as coronavirus disease 2019 (COVID-19) vaccines (3-5). In addition, virus-like particles (VLPs) are used as mRNA-delivery tools to combine the high infection efficiency of viral vectors and the transient nature of introduced mRNA (6-10). However, systemic injection of mRNA-delivering LNPs and VLPs was found to target mainly liver (11), with low efficiency for delivery into many other tissues, particularly the central nervous system (CNS) due to the presence of blood-brain barrier (BBB). The use of naturally occurring and newly engineered AAV capsids is a promising strategy for targeting non-liver tissues, such as CNS (12), skeletal muscle (13), and heart (14). AAV is a small, non-enveloped virus that could package a single-stranded DNA (ssDNA) (15) and has been engineered for DNA delivery, by replacing all viral protein-coding sequences with the therapeutic gene expression cassette between two required packaging signals (inverted terminal repeat, ITR) (16). Unlike retroviruses-derived VLPs in which virus assembly and genome encapsidation occur simultaneously, synthesized AAV genome is pumped into a pre-assembled capsid in a 3′ to 5′ direction via the viral DNA helicase/ATPase activity of Rep proteins (Rep78, Rep68, Rep52, and Rep40) as the motor (17, 18). Non-structural Rep78/68 proteins also serve as the ‘bridge’ between the ssDNA genome and the pre-assembled AAV capsid during virus packaging (FIG. 22A) (15, 18). Based on these findings, the inventor hypothesized that replacing ITR with RNA packaging signals (RPSs) and enabling the binding of Rep78/68 proteins to RPS-harboring RNAs may convert the AAV into an RNA-packaging virus.

On the “left side” of the vector genome of natural AAV virus, there are two promoters called p5 and p19, from which two overlapping messenger ribonucleic acids (mRNAs) of different length can be produced. Each of these contains an intron which can be either spliced out or not. Given these possibilities, four various mRNAs, and consequently four various Rep proteins with overlapping sequence can be synthesized. Their names depict their sizes in kilodaltons (kDa): Rep78, Rep68, Rep52 and Rep40. Rep78 and 68 can specifically bind the hairpin formed by the ITR in the self-priming act and cleave at a specific region, designated terminal resolution site, within the hairpin. They were also shown to be necessary for the AAVS1-specific integration of the AAV genome. All four Rep proteins were shown to bind ATP and to possess helicase activity. It was also shown that they upregulate the transcription from the p40 promoter, but downregulate both p5 and p19 promoters.

The disclosure provides an RAAV packaging system with increase RNA packaging ability (efficiency) and/or reduce DNA packaging ability (efficiency), at least in part by using a Rep protein containing a mutated helicase domain believed to have increased unwinding RNA property and/or have decreased DNA unwinding property.

In an aspect, the disclosure provides a Rep (e.g., Rep78, Rep68, Rep52, Rep40) protein comprising a helicase domain comprising an amino acid mutation relative to a reference helicase domain (e.g., SEQ ID NO: 186).

In another aspect, the disclosure provides a polynucleotide encoding a Rep (e.g., Rep78, Rep68, Rep52, Rep40) protein comprising a helicase domain comprising an amino acid mutation relative to a reference helicase domain (e.g., SEQ ID NO: 186).

In yet another aspect, the disclosure provides a polynucleotide encoding a Rep78 protein, a Rep68 protein, a Rep52 protein, and a Rep40 protein, wherein the Rep78 protein, the Rep68 protein, the Rep52 protein, and the Rep40 protein share a helicase domain comprising an amino acid mutation relative to a reference helicase domain (e.g., SEQ ID NO: 186).

In yet another aspect, the disclosure provides a helicase comprising a helicase domain comprising an amino acid mutation relative to a reference helicase domain (e.g., SEQ ID NO: 186).

In yet another aspect, the disclosure provides a polynucleotide encoding a helicase comprising a helicase domain comprising an amino acid mutation relative to a reference helicase domain (e.g., SEQ ID NO: 186).

In yet another aspect, the disclosure provides an RAAV packaging system, and specifically, a system for packaging an RNA into an AAV capsid to produce a recombinant RNA-packaged AAV particle (rRAAV particle), wherein the RNA comprises:
(a) an RNA sequence of interest (RSI), e.g., an RNA sequence encoding a protein of interest, and (b) an RNA-packaging signal (RPS) capable of interacting, e.g., binding, directly or indirectly, with an RPS-interacting molecule that facilitates packaging of the RNA into the AAV capsid;
wherein the system comprises:
(1) one or more capsid proteins (e.g., VP1, VP2, and/or VP3) for assembling the AAV capsid, or one or more coding sequences therefor, or a polynucleotide comprising said coding sequences;
(2) one or more Rep proteins (e.g., Rep78, Rep68, Rep52, Rep40) comprising a helicase domain comprising an amino acid mutation relative to a reference helicase domain (e.g., SEQ ID NO: 186), or one or more coding sequences therefor, or a polynucleotide comprising said coding sequences;
(3) the RPS-interacting molecule, or a coding sequence therefor, or a polynucleotide comprising said coding sequence;
(4) the RNA, or a coding sequence therefor, or a polynucleotide comprising said coding sequence, e.g., a transgene vector comprising or encoding the RNA; and
(5) optionally, one or more helper proteins required for AAV packaging (e.g., helper proteins from adenoviral E2a, E4, and/or VA genes), or one or more coding sequences therefor, or a polynucleotide comprising said coding sequences.

In yet another aspect, the disclosure provides an RAAV packaging method, and specifically, a method for the production of a recombinant RNA-packaged AAV particle (rRAAV particle), said method comprising:
a) culturing for a sufficient time a cell comprising a system for packaging an RNA into a AAV capsid to produce the recombinant RNA-packaged AAV particle (rRAAV particle), and
b) harvesting the rRAAV particle or a population thereof;
wherein the RNA comprises:
(a) an RNA sequence of interest (RSI), e.g., an RNA sequence encoding a protein of interest, and
(b) an RNA-packaging signal (RPS) capable of interacting, e.g., binding, directly or indirectly, with an RPS-interacting molecule that facilitates packaging of the RNA into the AAV capsid;
wherein the system comprises:
(1) one or more capsid proteins (e.g., VP1, VP2, and/or VP3) for assembling the AAV capsid, or one or more coding sequences therefor, or a polynucleotide comprising said coding sequences;
(2) one or more Rep proteins (e.g., Rep78, Rep68, Rep52, Rep40) comprising a helicase domain comprising an amino acid mutation relative to a reference helicase domain (e.g., SEQ ID NO: 186), or one or more coding sequences therefor, or a polynucleotide comprising said coding sequences;
(3) the RPS-interacting molecule, or a coding sequence therefor, or a polynucleotide comprising said coding sequence;
(4) the RNA, or a coding sequence therefor, or a polynucleotide comprising said coding sequence, e.g., a transgene vector comprising or encoding the RNA; and
(5) optionally, one or more helper proteins required for AAV packaging (e.g., helper proteins from adenoviral E2a, E4, and/or VA genes), or one or more coding sequences therefor, or a polynucleotide comprising said coding sequences.

In yet another aspect, the disclosure provides use of a Rep protein (e.g., Rep78, Rep68, Rep52, Rep40) comprising a helicase domain comprising an amino acid mutation relative to a reference helicase domain (e.g., SEQ ID NO: 186), or a polynucleotide encoding the Rep protein, in the production of a recombinant RNA-packaged AAV particle (rRAAV particle), said production comprising:
a) culturing for a sufficient time a cell comprising a system for packaging an RNA into a AAV capsid to produce the recombinant RNA-packaged AAV particle (rRAAV particle), and
b) harvesting the rRAAV particle or a population thereof;
wherein the RNA comprises:
(a) an RNA sequence of interest (RSI), e.g., an RNA sequence encoding a protein of interest, and
(b) an RNA-packaging signal (RPS) capable of interacting, e.g., binding, directly or indirectly, with an RPS-interacting molecule that facilitates packaging of the RNA into the AAV capsid;
wherein the system comprises:
(1) one or more capsid proteins (e.g., VP1, VP2, and/or VP3) for assembling the AAV capsid, or one or more coding sequences therefor, or a polynucleotide comprising said coding sequences;
(2) one or more said Rep proteins (e.g., Rep78, Rep68, Rep52, Rep40), or one or more coding sequences therefor, or a polynucleotide comprising said coding sequences;
(3) the RPS-interacting molecule, or a coding sequence therefor, or a polynucleotide comprising said coding sequence;
(4) the RNA, or a coding sequence therefor, or a polynucleotide comprising said coding sequence, e.g., a transgene vector comprising or encoding the RNA; and
(5) optionally, one or more helper proteins required for AAV packaging (e.g., helper proteins from adenoviral E2a, E4, and/or VA genes), or one or more coding sequences therefor, or a polynucleotide comprising said coding sequences.

In some embodiments, the amino acid mutation leads to an increased RNA unwinding property and/or a decreased DNA unwinding property of the helicase or the Rep protein comprising the amino acid mutation.

The RNA unwinding property refers to the capability of the helicase domain of the disclosure to recognize and unwind an RNA to allow the unwound RNA to be packaged into a AAV capsid. The DNA unwinding property refers to the capability of the helicase domain of the disclosure to recognize and unwind a ssDNA to allow the unwound ssDNA to be packaged into a AAV capsid. Since natural AAV is a DNA virus with ssDNA vector genome, it is believed the helicase domain contained in wild type Rep proteins is capable of unwinding DNA. On the other hand, it was found in the Examples of the disclosure that RNA can be packaged into AAV capsid in the absence of engineering of wild type Rep proteins, demonstrating that wild type Rep proteins can also unwind RNA. For the purpose of the disclosure, reference Rep protein (e.g., wild type Rep protein) is engineered to increase the RNA unwinding property and/or decrease the DNA unwinding property of the helicase domain contained in the reference Rep protein.

The RNA unwinding property may be measured with any suitable measurement known in the art. Alternatively, the RNA unwinding property may be indicated by the RNA packaging efficiency of the RAAV packaging system or method of the disclosure, since it is believed that the packaging of vector genome utilizes the unwinding property of the helicase domain contained in the Rep protein. Thus, the increased or decreased RNA unwinding property may be indicated by the increased or decreased RNA packaging efficiency of the RAAV packaging system or method of the disclosure with the Rep protein of the disclosure comprising the amino acid mutation compared with an otherwise identical control RAAV packaging system or method without the amino acid mutation.

The DNA unwinding property may be measured with any suitable measurement known in the art. Alternatively, the DNA unwinding property may be indicated by the DNA packaging efficiency of the RAAV packaging system or method of the disclosure. Thus, the increased or decreased DNA unwinding property may be indicated by the increased or decreased DNA packaging efficiency of the RAAV packaging system or method of the disclosure with the Rep protein of the disclosure comprising the amino acid mutation compared with an otherwise identical control RAAV packaging system or method without the amino acid mutation.

In some embodiments, the amino acid mutation leads to increased RNA packaging efficiency and/or decreased DNA packaging efficiency. The increased or decreased RNA or DNA packaging efficiency refers to the increased or decreased RNA or DNA packaging ability (efficiency) of the RAAV packaging system or method of the disclosure with the Rep protein of the disclosure comprising the amino acid mutation compared with an otherwise identical control RAAV packaging system or method without the amino acid mutation. The Examples of the disclosure provides specific examples and details for the measurement of the RNA or DNA packaging ability (efficiency) of the RAAV packaging system or method of the disclosure.

The amino acid substitution of the disclosure may be introduced in various reference helicase domains as shown in the disclosure, including but not limited to the helicase domain (SEQ ID NO: 186) of wild type Rep proteins of wild type AAV2.

In some embodiments, the reference helicase domain is the helicase domain of a reference helicase, e.g., a wild type helicase.

In some embodiments, the reference helicase is a superfamily 3 (SF3) helicase.

In some embodiments, the reference helicase is a helicase capable of unwinding DNA.

In some embodiments, the reference helicase is a superfamily 3 (SF3) helicase capable of unwinding DNA.

In some embodiments, the reference helicase domain is the helicase domain of a reference Rep protein, e.g., a wild type Rep protein.

In some embodiments, the reference Rep protein is a reference Rep78 protein, a reference Rep68 protein, a reference Rep52 protein, or a reference Rep40 protein.

In some embodiments, the reference helicase domain is the reference helicase domain shared by a reference Rep78 protein, a reference Rep68 protein, a reference Rep52 protein, and a reference Rep40 protein of a same AAV virus.

In some embodiments, the reference Rep protein, the reference Rep78 protein, the reference Rep68 protein, the reference Rep52 protein, and the reference Rep40 protein are from a wild type AAV virus.

In some embodiments, the wild type AAV virus has a serotype selected from the group consisting of AAV1, AAV2, AAV3A, AAV3B, AAV4, AAV5, AAV6, AAV7, AAV8, AAV9, AAV10, AAV11, AAV12, AAV13, AAVrh74, AAVrh10, AAV-DJ, AAV.PHP.eB, Anc80L65, Anc80L65AAP, and 7m8.

In some embodiments, the reference helicase domain (e.g., SEQ ID NO: 186) comprises, from N- to C-terminus, Motif A, Motif B, Motif B′, Motif C, and Arginine Finger (R finger).

In some embodiments, the Motif A comprises, consists essentially of, or consists the amino acids at positions corresponding to position 329 through position 342 of the amino acid sequence of SEQ ID NO: 186, wherein the position is numbered according to SEQ ID NO: 88.

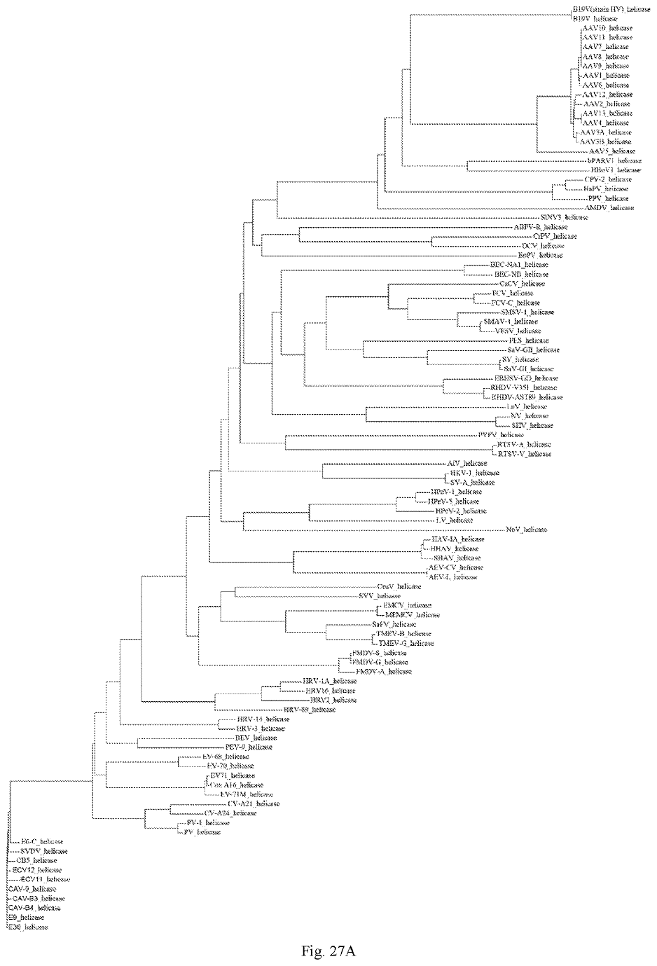

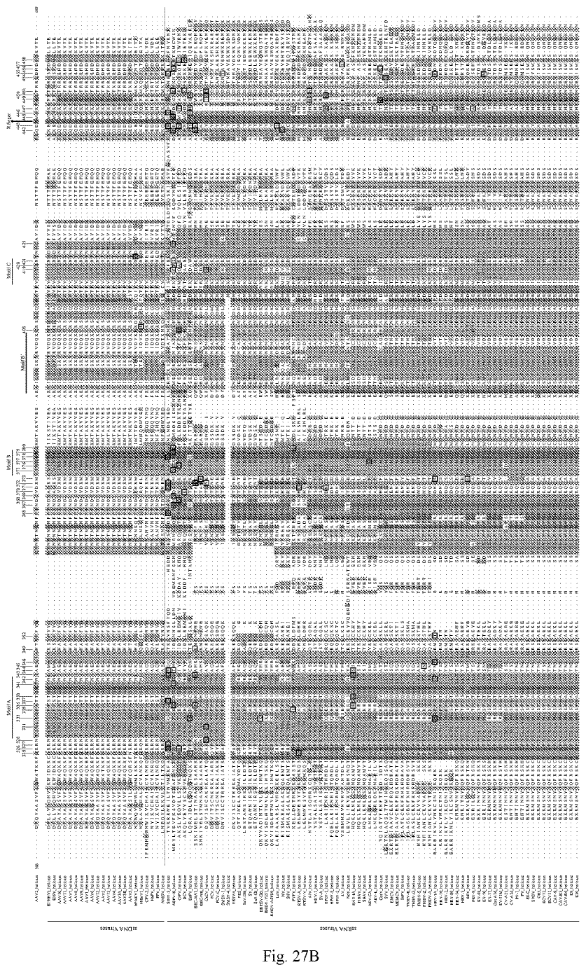

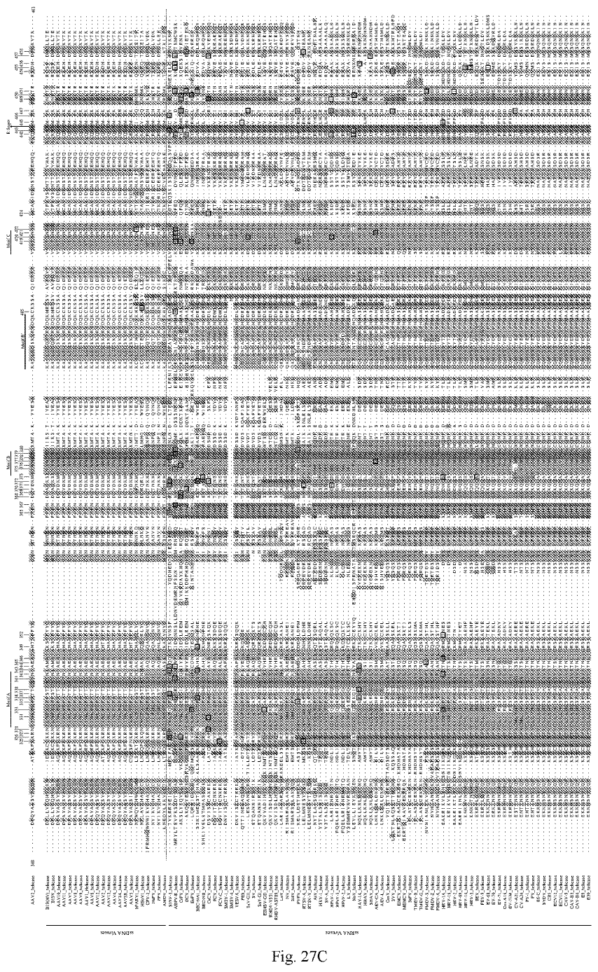

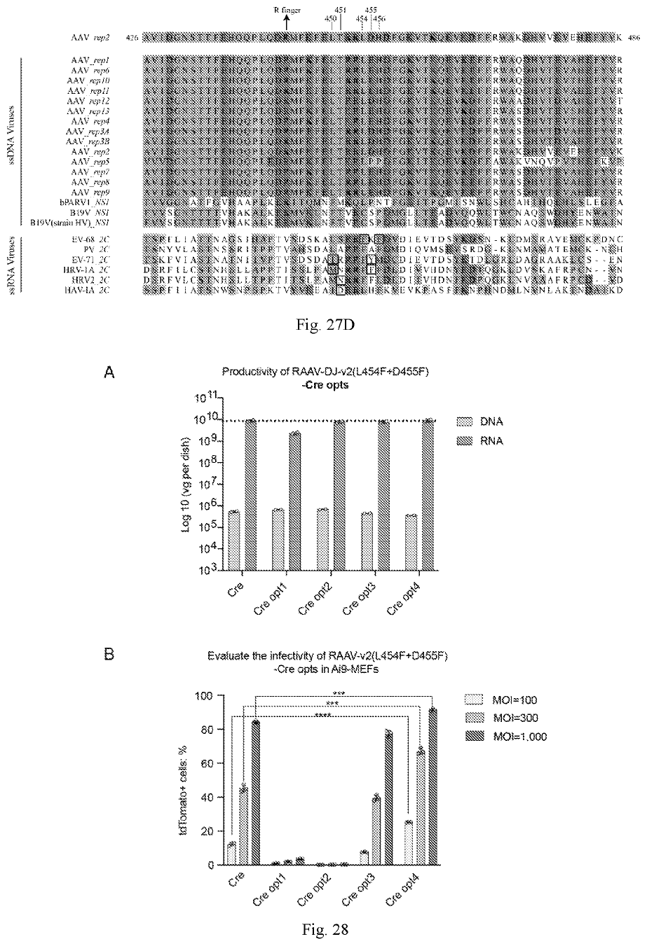

As used in the disclosure, “a position corresponding to a position” or “positions corresponding to positions” can be determined by sequence alignment. Exemplary sequence alignment of helicase domains or viral proteins are shown in FIG. 27A-27D. For example, a position of a Rep protein of the disclosure corresponding to position A344 of SEQ ID NO: 88 may be position A344 of AAV2 Rep78 of SEQ ID NO: 88, wherein the position is numbered according to SEQ ID NO: 88, or A346 of AAV8 Rep78 of SEQ ID NO: 94, wherein the position is numbered according to SEQ ID NO: 94. For another example, a position of a helicase domain of the disclosure corresponding to position A344 of SEQ ID NO: 186 may be position A344 of the AAV8 helicase domain of SEQ ID NO: 186, wherein the position is numbered according to SEQ ID NO: 88, or A346 of the AAV8 helicase domain of SEQ ID NO: 192, wherein the position is numbered according to SEQ ID NO: 94.

As used in the disclosure, “wherein the position is numbered according to SEQ ID NO: X” indicates how the indicated position is numbered. In the case that the Rep protein of the disclosure contains the helicase domain of the disclosure, a position in the helicase domain can be either numbered according to the Rep protein or numbered according to the helicase domain. For example, position A344 of AAV2 Rep78 of SEQ ID NO: 88 is numbered according to SEQ ID NO: 88; alternatively, position A344 can also be termed as position A37, in which case it is numbered according to SEQ ID NO: 186.

In some embodiments, the Motif B n comprises, consists essentially of, or consists the amino acids at positions corresponding to position 374 through position 379 of the amino acid sequence of SEQ ID NO: 186, wherein the position is numbered according to SEQ ID NO: 88.

In some embodiments, the Motif B′ comprises, consists essentially of, or consists the amino acids at positions corresponding to position 391 through position 404 of the amino acid sequence of SEQ ID NO: 186, wherein the position is numbered according to SEQ ID NO: 88.

In some embodiments, the Motif C comprises, consists essentially of, or consists the amino acids at positions corresponding to position 416 through 421 of the amino acid sequence of SEQ ID NO: 186, wherein the position is numbered according to SEQ ID NO: 88.

In some embodiments, the Arginine Finger (R finger) is Arginine at a position corresponding to position 444 of the amino acid sequence of SEQ ID NO: 186, wherein the position is numbered according to SEQ ID NO: 88.

In some embodiments, the reference Rep78 protein comprises, consists essentially of, or consists an amino acid sequence having a sequence identity of at least about 30% (e.g., at least about 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to the amino acid sequence of any one of SEQ ID NOs: 88-109; wherein the reference Rep68 protein comprises, consists essentially of, or consists of the amino acid sequence of SEQ ID NO: 284 or a corresponding amino acid sequence comprised in any one of SEQ ID NOs: 89-109; wherein the reference Rep52 protein comprises, consists essentially of, or consists of the amino acid sequence of SEQ ID NO: 285 or a corresponding amino acid sequence comprised in any one of SEQ ID NOs: 89-109; or wherein the reference Rep40 protein comprises, consists essentially of, or consists of the amino acid sequence of SEQ ID NO: 286 or a corresponding amino acid sequence comprised in any one of SEQ ID NOs: 89-109.

In some embodiments, the reference helicase domain comprises, consists essentially of, or consists an amino acid sequence having a sequence identity of at least about 30% (e.g., at least about 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to the amino acid sequence of any one of SEQ ID NOs: 186-207.

In some embodiments, the amino acid mutation is at a position corresponding to one or more positions of position 308 through position 463 (position 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463), optionally position 325 through position 461 (position 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461), of the amino acid sequence of any one of SEQ ID NOs: 186-207, wherein the position is numbered according to SEQ ID NO: 88.

In some embodiments, the amino acid mutation is at a position corresponding to the position of a conserved amino acid (e.g., a position corresponding to A344 of SEQ ID NO: 88) across at least 80%, at least 90%, or 100% of the Rep proteins (e.g., SEQ ID NOs: 88-109) of ssDNA viruses.

In some embodiments, the amino acid mutation is at a position corresponding to a position in one or more of Motif A, Motif B, Motif B′, Motif C of the reference helicase domain, a upstream region no more than about 30, 25, 20, 15, 10, or 5 amino acids from the N-terminal of any one of Motif A, Motif B, Motif B′, Motif C, and Arginine Finger (R finger) of the reference helicase domain, and a downstream region no more than about 30, 25, 20, 15, 10, or 5 amino acids from the C-terminal of any one of Motif A, Motif B, Motif B′, Motif C, and Arginine Finger (R finger) of the reference helicase domain.

In some embodiments, the Rep78 protein comprising said amino acid mutation comprises, consists essentially of, or consists an amino acid sequence having a sequence identity of at least about 30% (e.g., at least about 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9%) and less than 100% to the amino acid sequence of any one of SEQ ID NOs: 88-109.

In some embodiments, the helicase domain comprising said amino acid mutation comprises, consists essentially of, or consists an amino acid sequence having a sequence identity of at least about 30% (e.g., at least about 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9%) and less than 100% to the amino acid sequence of any one of SEQ ID NOs: 186-207.

In some embodiments, the amino acid mutation comprises a mutation at a position corresponding to G325, K326, R327, N328, W331, F333, P335, A336, T337, T338, T341, N342, I343, A344, E345, A346, H349, P352, P365, N367, D368, C369, V370, D371, K372, M373, I375, W376, W377, E378, E379, G380, C405, K406, T419, S420, N421, T422, M424, C425, Q442, D443, M445, F446, K447, E449, L450, T451, L454, D455, H456, D457, F458, and/or V461 of the amino acid sequence of SEQ ID NO: 186, wherein the position is numbered according to SEQ ID NO: 88.

In some embodiments, the amino acid mutation comprises a mutation at a position corresponding to G325, R327, W331, A336, T337, I343, A344, D371, K372, M373, I375, E378, C405, T419, S420, T422, C425, Q442, D443, M445, K447, E449, L450, T451, L454, D455, H456, D457, F458, and/or V461 of the amino acid sequence of SEQ ID NO: 186, wherein the position is numbered according to SEQ ID NO: 88.

In some embodiments, the amino acid mutation comprises a mutation at a position corresponding to A336, T337, I343, A344, K372, E378, D443, M445, K447, E449, L450, T451, L454, D455, H456, D457, F458, and/or V461 of the amino acid sequence of SEQ ID NO: 186, wherein the position is numbered according to SEQ ID NO: 88.

In some embodiments, the amino acid mutation comprises a substitution.

In some embodiments, the amino acid mutation comprises a conservative substitution or a non-conservative substitution.

In some embodiments, the amino acid mutation comprises a substitution with a non-polar amino acid residue (such as, Glycine (Gly/G), Alanine (Ala/A), Valine (Val/V), Cysteine (Cys/C), Proline (Pro/P), Leucine (Leu/L), Isoleucine (Ile/I), Methionine (Met/M), Tryptophan (Trp/W), Phenylalanine (Phe/F), a polar amino acid residue (such as, Serine (Ser/S), Threonine (Thr/T), Tyrosine (Tyr/Y), Asparagine (Asn/N), Glutamine (Gln/Q)), a positively charged amino acid residue (such as, Lysine (Lys/K), Arginine (Arg/R), Histidine (His/H)), or a negatively charged amino acid residue (such as, Aspartic Acid (Asp/D), Glutamic Acid (Glue/E)).

In some embodiments, the amino acid mutation comprises a substitution corresponding to a substitution selected from the group consisting of G325P, G325I, K326E, K326R, R327P, N328V, W331I, F333H, F333Y, F333K, P335S, A336P, A336S, A336R, T337G, T338G, T341S, N342I, I343T, I343A, I343L, A344T, A344V, A344S, E345N, A346F, H349K, P352T, P365Y, N367D, D368G, C369Y, V370K, D371Q, D371G, D371N, K372Q, K372E, K372N, M373S, M373E, M373A, I375V, W376I, W377M, E378D, E379D, G380L, G380F, C405H, K406R, T419S, T419F, T419I, T419L, S420A, S420M, S420C, N421T, N421S, T422H, T422S, M424N, C425I, Q442K, Q442F, Q442H, Q442R, Q442L, Q442V, D443S, D443R, D443Y, D443N, D443A, M445I, M445R, M445F, F446R, F446I, K447F, K447N, K447H, K447P, K447T, K447A, E449D, E449I, E449R, L450M, L450I, L450V, T451D, T451I, T451E, T451K, T451N, L454F, L454V, D455F, D455K, D455H, D455Y, D455T, D455M, H456F, H456D, H456S, D457E, D457S, D457F, F458Y, F458K, V461L, and a combination thereof, wherein the position is numbered according to SEQ ID NO: 88.

In some embodiments, the amino acid mutation comprises a substitution corresponding to a substitution selected from the group consisting of G325P, R327P, W331I, A336P, A336S, A336R, T337G, I343T, I343A, I343L, A344T, A344V, D371Q, K372Q, K372E, K372N, M373S, I375V, E378D, C405H, T419S, S420A, T422H, T422S, C425I, Q442H, Q442R, D443S, D443Y, D443N, D443A, M445I, K447F, K447N, K447T, E449D, L450M, L450I, L450V, T451D, T451E, L454F, D455F, D455Y, D455T, D455M, H456D, H456S, D457E, D457S, D457F, F458Y, V461L, and a combination thereof, wherein the position is numbered according to SEQ ID NO: 88.

In some embodiments, the amino acid mutation comprises a substitution corresponding to a substitution selected from the group consisting of A336P, T337G, I343T, A344T, A344V, K372Q, E378D, D443S, M445I, K447F, E449D, L450M, T451D, L454F, D455F, D455T, H456D, D457E, F458Y, V461L, and a combination thereof, wherein the position is numbered according to SEQ ID NO: 88.

In some embodiments, the amino acid mutation comprises or consists of a combination substitution corresponding a combination substitution selected from the group consisting of A336P+T337G, K372Q+E378D, D443S+M445I, D443S+L454F+D455F, K447F+E449D+T451D, K447F+L450M, K447F+F458Y, K447F+H456D+F458Y, K447F+V461L, E449D+L450M, L450M+T451D, L454F+D455F, D455T+H456D+D457E+F458Y, H456D+D457E+F458Y, F458Y+V461L, A344T+K372Q, A336P+A344T+K447F, A336P+A344V+K447F, I343T+K447F, T343T+L450M, A344T+K447F, A344V+K447F, A344T+L450M, A344V+L450M, A344T+K447F+E449D+T451D, K372Q+K447F, K372Q+L450M, K372Q++K447F+E449D+T451D, K372Q+V461L, E378D+K447F, E378D+L450M, E378D++K447F+E449D+T451D, A344T+K372Q+K447F, A344V+K372Q+K447F, and a combination thereof, wherein the position is numbered according to SEQ ID NO: 88.

In some embodiments, the amino acid mutation comprises or consists of a combination substitution corresponding a combination substitution of A344V and K447F, wherein the position is numbered according to SEQ ID NO: 88. For example, the combination substitution corresponding a combination substitution of A344V and K447F may be a combination substitution of A346V and K449F, wherein the position is numbered according to SEQ ID NO: 94 In some embodiments, the helicase domain comprising said amino acid mutation comprises, consists essentially of, or consists of the amino acid sequence of SEQ ID NO: 287. In this case, the reference helicase domain is the helicase domain (SEQ ID NO: 186) of the wild type Rep proteins of AAV2.

In some embodiments, the Rep protein comprising said amino acid mutation comprises, consists essentially of, or consists of the amino acid sequence of SEQ ID NO: 288. In this case, the reference helicase domain is the helicase domain (SEQ ID NO: 186) of the wild type Rep proteins of AAV2.

In some embodiments, the helicase domain comprising said amino acid mutation comprises, consists essentially of, or consists of the amino acid sequence of SEQ ID NO: 290. In this case, the reference helicase domain is the helicase domain (SEQ ID NO: 192) of the wild type Rep proteins of AAV8.

In some embodiments, the Rep protein comprising said amino acid mutation comprises, consists essentially of, or consists of the amino acid sequence of SEQ ID NO: 291. In this case, the reference helicase domain is the helicase domain (SEQ ID NO: 192) of the wild type Rep proteins of AAV8.

In some embodiments, the Rep protein comprise a mutation partially or substantially abolishing the endonuclease activity of the Rep protein, e.g., in the Original Binding Domain (OBD) of the Rep protein, optionally, the mutation comprises or consists of a mutation corresponding to a Y156F mutation, a K146A+D149A+E150A mutation (KDE-mu), or an E83A+K84A+E86A mutation (EKE-mu), wherein the position is numbered according to SEQ ID NO: 88.

In some embodiments, Rep protein comprises a combination substitution comprising or consisting of Y156F, A344V, and K447F, wherein the position is numbered according to SEQ ID NO: 88.

In some embodiments, the Rep protein comprise the amino acid sequence of SEQ ID NO: 289.

In some embodiments, the RPS-interacting molecule is said Rep protein.

In some embodiments, the RPS-interacting molecule comprises an RPS-binding protein (RPSBP) capable of binding directly or indirectly to the RNA packaging signal (RPS).

In some embodiments, the Rep protein is (e.g., N-terminally, C-terminally, internally) fused with the RPSBP, optionally, via a peptide linker.

In some embodiments, the RPS is located at or near the 5′ end of the RSI, at or near the 3′ end of the RSI, or internal to the RSI.

In some embodiments, the RNA comprises one, two, or three copies of the RPS.

In some embodiments, the RPS comprises an MS2 sequence (e.g., SEQ ID NO: 54), an PP7 binding site (e.g., SEQ ID NO: 56), and/or a Com binding site (e.g., SEQ ID NO: 58).

In some embodiments, (a) the RPS comprises an MS2 sequence (e.g., SEQ ID NO: 54), and the RPSBP comprises a bacteriophage-derived MS2 coat protein (MCP) (e.g., SEQ ID NO: 49); (b) the RPS comprises an PP7 binding site (e.g., SEQ ID NO: 56), and the RPSBP comprises a PP7 bacteriophage coat protein (PCP) (e.g., SEQ ID NO: 51), or (c) the RPS comprises a Com binding site (e.g., SEQ ID NO: 58), and the RPSBP comprises a phage COM protein (COM) (e.g., SEQ ID NO: 53).

In some embodiments, the RNA, or a coding sequence therefor, or a polynucleotide comprising said coding sequence, e.g., a transgene vector comprising or encoding the RNA, lacks a functional DNA packaging signal, e.g., an AAV ITR (such as, 5′ AAV2 ITR and/or 3′ AAV2 ITR), or a coding sequence therefor.

In some embodiments, the RNA is transcribed from a polynucleotide (e.g., a transgene plasmid) lacking a functional DNA packaging signal, e.g., an AAV ITR (such as, 5′ AAV2 ITR and/or 3′ AAV2 ITR), or a coding sequence therefor.

In some embodiments, the AAV capsid comprises a capsid from an AAV having a serotype selected from the group consisting of AAV1, AAV2, AAV3A, AAV3B, AAV4, AAV5, AAV6, AAV7, AAV8, AAV9, AAV10, AAV11, AAV12, AAV13, AAVrh74, AAVrh10, AAV-DJ, AAV.PHP.eB, Anc80L65, Anc80L65AAP, and 7m8.

In some embodiments, the RNA is not bound to the AAV capsid.

In some embodiments, the RSI is a RNA coding sequence for a gene of interest (GOI), a protein (e.g., a therapeutic protein, an antigen protein, or a gene-editing protein such as a CRISPR/Cas effector enzyme (“a Cas protein” for short), a ZFN protein, a TALEN protein)-encoding RNA, such as, a mRNA, or a non-coding, functional RNA (such as, a transfer RNA (tRNA), a ribosomal RNA (rRNA), a small interfering RNA (siRNA), a short hairpin RNA (shRNA), an antisense RNA, an antisense oligonucleotide, a micro RNA (miRNA), or an RNA component of a CRISPR-Cas (e.g., Cas9, Cas12, Cas13) system, including a guide RNA (or a gRNA), such as, a single guide RNA (or a sgRNA, a chimeric RNA, an RNA chimera), a CRISPR RNA (crRNA), and a tracr RNA), or a precursor thereof.

In some embodiments, the GOI comprises a protein (e.g., a fluorescent protein, a therapeutic protein, an antigen protein, or a gene-editing protein such as a Cas protein, a ZFN protein, a TALEN protein), an enzyme (such as a Cre protein, or a CRISPR/Cas effector enzyme, e.g., Cas9, Cas12, Cas13, or a variant thereof), a structural protein, an mRNA, a non-coding RNA (ncRNA), an siRNA, a piRNA, a short hairpin RNA or shRNA, a microRNA (miRNA) or a precursor thereof (including pre-miRNA and pri-miRNA), a ribosomal RNA (rRNA), an antisense sequence or oligonucleotide (ASO), an RNA component of a CRISPR-Cas system, including a guide RNA (or a gRNA), such as, a single guide RNA (or a sgRNA, a chimeric RNA, an RNA chimera), a CRISPR RNA (crRNA), and a tracr RNA, a guide RNA or gRNA for a CRISPR/Cas effector enzyme, an rRNA, a tRNA, a snoRNA, a snRNA, an exRNA, a scaRNA, a lncRNA, a Xist, and a HOTAIR.

In yet another aspect, the disclosure provides a vector comprising the polynucleotide of the disclosure; optionally, wherein the vector is a plasmid.

In yet another aspect, the disclosure provides a cell, an isolated cell, a host cell, or an isolated host cell that comprises the Rep protein, the helicase, the polynucleotide, the system, or the vector of the disclosure.

In yet another aspect, the disclosure provides a recombinant RNA-packaged AAV particle (rRAAV particle) or a population thereof produced by the method of the disclosure.

In an aspect, the invention described herein provides a recombinant viral particle comprising a DNA virus protein shell, and a “vector genome” comprising RNA, such as single-stranded RNA (rather than DNA). The “vector genome” may not be a typical viral RNA, in that it may have very little, if any, virus-originated sequences, other than the RNA Packaging Signal (RPS) described herein below. That is, the DNA virus normally or naturally encapsidates a DNA viral vector genome inside the protein shell, while the recombinant version of the DNA virus viral particle as described herein encapsidates instead an RNA.

By “RNA” or “ribonucleic acid” it means a stretch of ribonucleotides each composed of a phosphate, a ribose, and a base (A (adenine), U (uracil), G (guanine), or C (cytosine)), each of which ribonucleotides may be modified (for example, base-modified, glycosyl-modified, phosphate-modified, e.g., oxygen-modified, fluorine-modified, sulphur-modified, pseudo-modified (e.g., pseudo-uridine-modified), methylated, capped (e.g., 5-capped)) or unmodified, and, optionally, fused directly or indirectly with a stretch of deoxyribonucleotides each composed of a phosphate, a deoxyribose, and a base (A (adenine), T (thymine), G (guanine), or C (cytosine)), each of which deoxyribonucleotides may be modified (for example, base-modified, glycosyl-modified, phosphate-modified, e.g., oxygen-modified, fluorine-modified, sulphur-modified, pseudo-modified, methylated, capped (e.g., 5-capped)) or unmodified, e.g., a RNA-DNA chimera, a DNA-RNA-DNA chimera, a RNA-DNA-RNA chimera.

A typical (non-limiting) example of such a recombinant DNA virus viral particle is adeno-associated virus (AAV), which normally/naturally encapsidates a single-stranded DNA (ssDNA) vector genome. Another non-limiting example of such DNA virus is an oncolytic DNA virus, such as an oncolytic herpes virus (e.g., herpes simplex virus or HSV), an oncolytic adenovirus, a vaccinia virus (VACV), vesicular stomatitis virus (VSV), etc.

The invention is partly based on the surprising discovery that, transcribed AAV ITR, in RNA form, can facilitate high efficiency direct packaging of transcribed RNA encompassing such transcribed AAV ITR into conventional AAV viral particles.

The invention described herein is also partly based on the surprising discovery that, other than the transcribed AAV ITR (RNA), certain artificial or heterologous RNA sequences and their cognate/corresponding/native RNA binding proteins can also serve as pairs of RNA Packaging Signals (RPS) and RPS-Interacting Proteins (RPSIPs) to replace the function of wild-type packaging signal sequences and interacting proteins useful for DNA virus packaging, thus packaging an RNA into a DNA virus protein shell that normally/naturally encapsidates a DNA vector genome.

For example, in wild-type AAV, the ITR sequences at the 5′ and 3′ ends of the DNA vector genome comprise sequence elements such as Rep-Binding Element (RBE) and RBE′ that can interact with the Rep proteins (such as Rep68 and Rep78). The Rep proteins bind the ITR and facilitate the packaging of AAV ssDNA vector genome comprising such ITR sequence elements into the AAV viral particle.

The inventors have discovered that, by providing, as an RPS, a transcribed ITR sequence, and/or an artificial or heterologous RNA sequence, such as the MS2 sequence, to an RNA sequence of interest (RSI), the resulting RNA sequence comprised of the RPS and the RSI can be efficiently packaged into an AAV viral protein shell in the presence of MCP—the bacteriophage-derived MS2 coat protein (MCP) that naturally binds MS2. The ability of the artificial RPS/RPSIP pair—e.g., MS2/MCP- to facilitate RNA packaging into a DNA virus protein shell, does not depend on the presence of, but can function independently of, the native ITR packaging signal for DNA packaging. In a sense, the heterologous MS2-MCP pair constitutes an artificial system of RPS and RPSIP pair that can effectively replace the natural ITR-Rep DNA packaging system, with the former efficiently facilitates RNA packaging. Such RNA-containing DNA virus, such as AAV, maybe referred herein as R-DNA viral particle (or RAAV in the case of AAV), or recombinant R-DNA viral particle (or rRAAV in the case of AAV).

The R-DNA viral particle and RAAV viral particles of the disclosure can be used to deliver the RNA transcript of any transgene or gene of interest (GOI) of suitable length (e.g., within the packaging limit of the various DNA virus or AAVs) or any guide RNA to a host cell compatible with the tropism of the DNA viral protein shell or AAV viral capsid shell. As used herein, the recombinant DNA viral particles such as recombinant AAV vectors, vector genomes, and recombinant AAV viral particles or recombinant AAV particles, are referred to herein as rRAAV vectors (recombinant RNA adeno-associated virus vectors), vector genomes, and recombinant RAAV (rRAAV) viral particles or rRAAV particles, respectively (the “rRAAV vectors” and “rRAAV particles” are used exchangeably herein).

Specifically, on the one hand, just like any normal or conventional AAV vectors, the subject RAAV vectors can also be composed of any of the same capsid shells found in any wild-type AAVs carrying DNA as the viral genetic material. Thus, the subject RAAV vectors possess all the usual advantages derived from the AAV shell, such as specific/broad tropism and low immugenicity.

However, on the other hand, the genome of the subject RAAV vectors are comprised of RNAs (e.g., mRNAs), which have short lifespans, and thereby leading to a transient expression of any encoded gene product on such RNA genetic material.

Such transient expression is desired in at least some cases. For example, the RAAV vectors of the disclosure are advantageous for in vivo DNA gene editing, since time-restricted exposure to RAAV-encoded DNA gene editors (such as the mRNA coding sequence for a CRISPR/Cas system effector enzyme Cas9 and variants thereof fused to a base editor) may enable efficient gene editing. Such transiently expressed DNA editors also improves the safety profile of the gene therapy, by reducing off-target gene targeting, and reducing immunogenicity compared to the persistent expression of the same DNA gene editors expressed from conventional DNA-based AAV vectors.

In addition, compared to traditional DNA-based AAV vectors, the subject RAAV vectors can carry longer transgenes, because of the exclusion of at least the promoter (and also any non-transcribed enhancer sequences that may be) required for expression of the GOI encoded by a DNA-based AAV vector.

Although the subject rRAAV vectors have different sequence elements and organization compared to traditional DNA-based AAV vectors, the rRAAV viral particles have the same entry and intracellular-trafficking processes as the conventional DNA-based AAV vectors. However, they have quite different fates after entering into the host cell nucleus. After entering into the nucleus, the mRNA genome of the subject RAAV vector is released and subsequently transported to the cytoplasm, leading to translation. As is understood, mRNAs generally have short lifespans, ranging from several minutes to days, and are eventually degraded via many cellular mechanisms. However, the limited mRNA lifespan still enables the host cell to complete the protein synthesis, often without the delay due to the 2^{nd} strand cDNA synthesis in DNA-based AAV vectors, and allowing the encoded proteins to function rapidly.

Numerous such RPS/RPSIP pairs can be used for RNA packaging into DNA virus. The inventors have demonstrated at least two additional such pairs, including the PP7 sequence and the PP7 bacteriophage coat protein (PCP), and the com sequence and the phage COM protein (COM), that efficiently package RNA comprising the heterologous RPS (i.e., PP7 and com sequences, respectively). The three pairs of RPS/RPSIP as demonstrated encompass at least two categories. Unlike MS2/MCP and PP7/PCP that are natural viral packaging systems, com/COM is not a natural viral packaging system but known to be transcription regulators that play roles in the transcription initiation of the bacteriophage Mu mom gene. Numerous transcribed modified AAV ITR sequences can also be used as RPS of the disclosure.

The invention described herein is also not limited to a specific serotype of DNA virus (e.g., a specific AAV serotype). The inventors have demonstrated efficient packaging of RNA sequences with suitable RPS into representative AAV viruses including AAV5, AAV8, AAV9, and AAV-DJ, using in conjunction with compatible RPSIP in each case.

The invention described herein is also based on the discovery that the efficiency of packaging undesired DNA into natural DNA virus viral particles can be decreased by several independent approaches.

In certain embodiments, the undesired DNA packaging efficiency can be reduced by increasing the overall size of the DNA vector from which the RNA of interest is transcribed. For example, in the often used triple transfection method for AAV production, the gene of interest (GOI) can be carried by a first plasmid, the required Rep and Cap proteins are encoded by the rep and cap genes on a second plasmid, while the other AAV packaging required components are provided by a third plasmid. According to this embodiment of the disclosure, the RNA sequence to be packaged into the DNA virus can be transcribed from the first plasmid, and the overall size of the first plasmid can be artificially increased by including a random stuffer sequence (e.g., an intron), such as a stuffer sequence that is at least about 1 kb, 2 kb, 3 kb, 4 kb, 5 kb or more in length, or a stuffer sequence that increases the overall size of the first plasmid by 1 kb, 2 kb, 3 kb, 4 kb, 5 kb or more, e.g., to about 6 kb, 7 kb, 8 kb, 9 kb, 10 kb or more, etc.

In certain other embodiments, the undesired DNA packaging efficiency can be reduced by inhibiting the function of a canonical element that facilitates DNA packaging. Such a canonical element for DNA packaging may include a DNA sequence (such as an element of the AAV ITR sequence that facilitates DNA packaging, including the trs sequence, the RBE or RBE′ sequence, or the entire ITR sequence of an AAV); and/or a protein element participating in the DNA packaging, such as, a protein that interacts with the DNA sequence (such as a mutant Rep68 or Rep 78 protein that lacks or has diminished trs-endonuclease activity).

Thus, one aspect of the disclosure provides a ribonucleotide (RNA) sequence capable of being packaged into a DNA virus viral particle, such as a DNA virus that naturally packages DNA, wherein the RNA sequence comprises: (1) an RNA sequence of interest (RSI); and, (2) an RNA-packaging signal (RPS) capable of interacting, e.g., binding, directly or indirectly to an RPS-interacting molecule (e.g., an RPS-interacting protein or RPSIP) that facilitates packaging of the RNA sequence into the DNA virus viral particle.

Such an RNA sequence can comprise any RSI (RNA), which may be encoded by “a gene of interest” or “GOI” (DNA).

As used herein, “a gene of interest” or “GOI” includes any coding sequence for a protein or polypeptide, including intron and exon sequences, and/or coding sequence for any non-translated RNA or non-coding RNA (neRNA, such as siRNA, piRNA, short hairpin RNA or shRNA, microRNA or miRNA or precursors thereof including pre-miRNA and pri-miRNA, antisense sequence or oligonucleotide (ASO), guide RNA or gRNA for CRISPR/Cas, rRNA, tRNA, snoRNA, snRNA, exRNA, scaRNA, lncRNA, Xist, and HOTAIR, etc.).

Similarly, representative (non-limiting) RSI includes, for example, a protein (e.g., a therapeutic protein, an antigen protein, or a gene-editing protein such as a CRISPR/Cas effector enzyme (“a Cas protein” for short), a ZFN protein, a TALEN protein)-encoding RNA, such as an mRNA, or a non-coding, functional RNA (such as a transfer RNA (tRNA), a ribosomal RNA (rRNA), a transfer-messenger RNA (tmRNA), a small interfering RNA (siRNA), a short hairpin RNA (shRNA), an antisense RNA or oligonucleotide (ASO), a micro RNA (miRNA), an RNA aptamer, or an RNA component of a CRISPR-Cas (e.g., Cas9, Cas12, Cas13) system, such as, a single guide RNA (or an sgRNA, a chimeric RNA, an RNA chimera), a CRISPR RNA (crRNA) and a tracr RNA), or a precursor thereof, or an RNA component of a RISC complex or RNAi pathway (such as shRNA, miRNA, or siRNA), a regulatory RNA, Piwi-interacting RNAs (piRNAs), small nucleolar RNAs (snoRNAs), a long non-coding RNA (lncRNA) (including intergenic lincRNA, intronic ncRNA, and sense/antisense lncRNA), a long intervening/intergenic noncoding RNA (lincRNA), an enhancer RNA, a bacterial small RNA (sRNA), snRNA, exRNA, scaRNA, Xist, and HOTAIR, and a precursor thereof.

The RNA sequence of the disclosure or GOI can comprise one coding sequence, or more than one (e.g., 2, 3, 4, 5, 6, 7, 8, 9, 10 or more) coding sequences. The length of the coding sequence, or the combined length of all coding sequences, may be no more than the maximum length of RNA that can be packaged into a particular or chosen DNA virus viral particle (e.g., AAV viral particle), which can differ from one specific DNA virus (e.g., AAV) viral particle from another.

In certain embodiments, a DNA sequence encoding or corresponding to the RNA of the disclosure, or a reverse complement of the DNA sequence, has reduced, diminished, or substantially no capacity of being packaged into the DNA virus viral particle. For example, the DNA sequence may encode the RNA of the disclosure (e.g., the DNA sequence has the reverse complement sequence of the RNA of the disclosure). The DNA sequence may also correspond to the RNA of the disclosure, in that the DNA sequence has otherwise identical nucleotide sequence as the RNA of the disclosure, except that the DNA sequence has T's, instead of the U's in the RNA of the disclosure. Regardless, the DNA sequence or the reverse complement thereof may lack a functional DNA packaging signal for packaging into the DNA virus viral particle, such as an AAV ITR for AAV packaging, such that the DNA sequence or the reverse complement thereof (DNA) has reduced, diminished, or substantially no capacity of being packaged into the DNA virus viral particle.

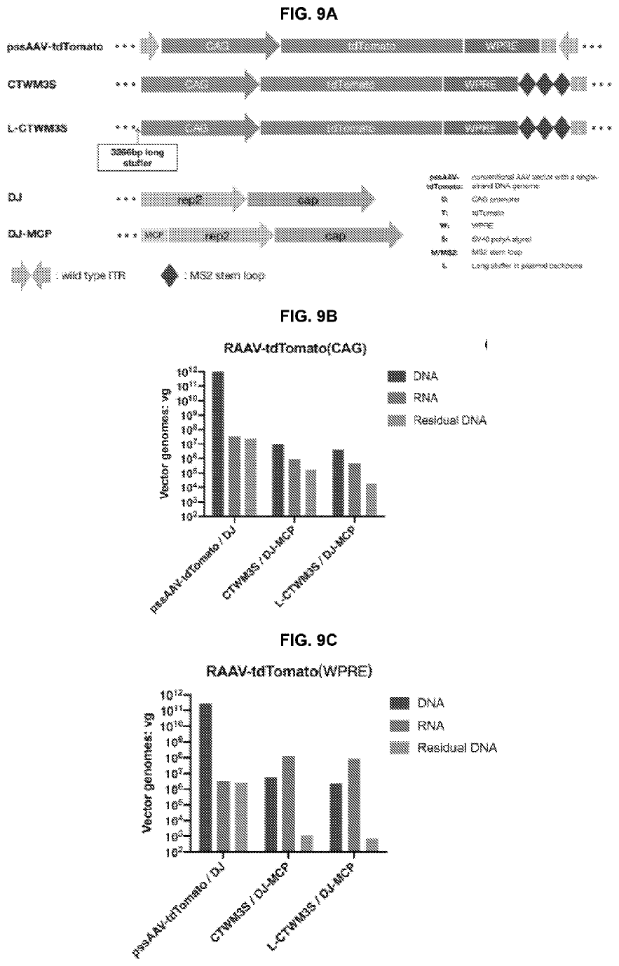

In certain embodiments, the RNA of the disclosure is transcribed from a DNA construct, such as transcribed from a DNA plasmid encoding the RNA sequence, wherein the DNA construct/plasmid comprises a stuffer sequence (e.g., an intron sequence) in its backbone sequence to enhance packaging of the RNA of the disclosure, and/or to reduce undesired packaging of DNA into the DNA virus viral particle. For example, the RNA of the disclosure can be transcribed from a DNA construct/plasmid, and the overall size of the DNA construct/plasmid can be artificially increased by including a random DNA stuffer sequence, such as a stuffer sequence that is at least about 1 kb, 2 kb, 3 kb, 4 kb, 5 kb or more in length, or a stuffer sequence that increases the overall size of the DNA construct/plasmid by 1 kb, 2 kb, 3 kb, 4 kb, 5 kb or more, e.g., to about 6 kb, 7 kb, 8 kb, 9 kb, 10 kb or more, etc. The stuffer sequence can be located upstream (e.g., immediately upstream) of the transcription unit comprising the coding sequence for the RNA of the disclosure (see FIG. 9A, in which a long stuffer sequence of >3 kb is inserted immediately upstream of a CAG promoter that drives the transcription of an exemplary RNA sequence of the disclosure). In certain embodiments, the stuffer sequence is inserted immediately upstream of a promoter operably linked to the codon sequence for the RNA of the disclosure. Optionally, in some embodiment, the coding sequence for the RNA of the disclosure is devoid of a functional natural DNA packaging signals for the DNA virus viral particle, such as devoid of a functional ITR sequence that supports packaging into an AAV viral particle.

In certain embodiments, the RNA of the disclosure is capable of being packaged into a DNA virus viral particle that is an AAV viral particle. Any AAV virus can be used to package the RNA of the disclosure, including, but not limited to, AAV1, AAV2, AAV3A, AAV3B, AAV4, AAV5, AAV6, AAV7, AAV8, AAV9, AAV10, AAV 11, AAV 12, AAV 13, AAVrh10, AAVrh74, AAVhu32, AAVhu37, AAV-DJ, AAV PHP.eB, Anc80L65, Anc80L65AAP, AAVrh74, or 7m8.

In certain embodiments, the RNA of the disclosure is capable of being packaged into a DNA virus viral particle that is an oncolytic viral particle. Exemplary (non-limiting) oncolytic viral particles include: oncolytic herpes virus (e.g., herpes simplex virus or HSV), an oncolytic adenovirus, a vaccinia virus (VACV), vesicular stomatitis virus (VSV), etc.

The location of the RPS in the RNA of the disclosure can be flexible. In certain embodiments, the RPS is located at or near the 5′ end of the RNA of the disclosure, at or near the 3′ end of the RNA of the disclosure, or internal to the RNA of the disclosure. In certain embodiments, the RPS is located at or near the 5′ end of the RNA sequence of interest (RSI), at or near the 3′ end of the RNA sequence of interest (RSI), or internal to the RNA sequence of interest (e.g., inside an intron of an mRNA).

There can be one or more RPS in the RNA of the disclosure. In certain embodiments, the RNA of the disclosure comprises more than one (e.g., 1, 2, 3, or more) RPS that are identical or substantially identical. In certain embodiments, the RNA of the disclosure comprises more than one (e.g., 1, 2, 3, or more) RPS, and at least two of which are different from each other.

In cases where more than one RPS are present on the RNA of the disclosure, at least two of the more than one RPS are adjacent to each other, such as in tandem, with an optional linker sequence in between. The linker between any two adjacent RPS sequences may be the same or different. The linker sequence may be a randomized RNA sequence with no substantial secondary structure, no known functional sequences or elements, and/or may be less than 50% in GC content. The length of the linker may be any where between 1-1 kb, 1-500 bases, 1-200 bases, 1 to about 100 bases, 1 to about 60 bases, about 5 to about 55 bases, about 10 to about 30 bases, or about 15-25 bases.

In certain embodiments, the RNA of the disclosure comprises 3 RPS sequences adjacent to one another, separated by two linker sequences, each independently about 20 or about 50 bases. For example, the first two of three identical RPS sequences may be separated by a linker of 20 bases, and/or the last two of the RPS sequences may be separated by a linker of 51 bases.

In certain embodiments, the RNA of the disclosure comprises more than one RPS (e.g., 1, 2, 3, 4, or 5 RPS), wherein at least two of the more than one RPS are not adjacent to each other. For example, one of the RPS may be located at the 5′ end of the RNA of the disclosure, while another RPS may be located at the 3′ end of the RNA of the disclosure, and an optional 3^{rd} RPS may be located inside an intron of an mRNA as the RSI within the RNA of the disclosure. A 4^{th} and/or a 5^{th} RPS may be located close or adjacent to any one the first, second, or third RPS.

In certain embodiments, the RNA of the disclosure comprises at least two (e.g., two or more) RPS sequences that are not adjacent to each other, e.g., one each located at or near one end of the RNA sequence of interest (RSI).

In certain embodiments, the RPS comprises a transcribed modified AAV inverted terminal repeat (ITR), wherein the transcribed modified AAV ITR (a) comprises a transcribed functional Rep-Binding Element (RBE), optionally further comprising a transcribed functional RBE′; and, (b) lacks either a transcribed terminal resolution site (TRS), or a transcribed reverse complement TRS (rcTRS), or both. In certain embodiments, the transcribed modified AAV ITR further comprises a transcribed D region sequence (D sequence or D′ sequence). In certain embodiments, the RPS-interacting molecule is Rep78, Rep68, Rep52, and/or Rep40.

As used herein, “AAV viral particle” includes viral particles comprising any wild-type capsids of adeno-associated virus (AAV) (belonging to the genus Dependoparvovirus, which in turn belongs to the family Parvoviridae), as well as engineered or variants thereof having modified sequence and/or tissue or host tropism.

As used herein, “intron” refers to a non-coding segment of a DNA or an RNA, which are normally removed a transcribed RNA through splicing. However, the RNA of the disclosure may comprise an intron sequence, such as an intron sequence from a heterologous gene (“heterologous” with respect to the gene of interest or GOI, which is to be expressed as a transgene delivered to a host cell by the rRAAV viral particle of the disclosure), in order to enhance the expression of the GOI. Such intron sequence in the RNA of the disclosure may or may not be removed by splicing. In addition, such intron sequence may further comprise a transcribed enhancer or a part thereof, since certain enhancers can be located within an intron of a coding DNA.

As used herein, “exon” refers to a coding segment of a DNA or an RNA, which exon is to be translated into a protein sequence. However, in certain embodiments, an exon sequence within the RNA of the disclosure may encode part of or the entirety of the GOI to be expressed as a transgene delivered to a host cell by the rRAAV viral particle of the disclosure. In other embodiments, an exon sequence within the RNA of the disclosure may belong to a heterologous gene (with respect to the GOI), and the presence of such exon may enhance the expression of the GOI.

As used herein, “coding sequence” includes a polynucleotide sequence of a DNA or an RNA which encodes a product that can be (a) a protein or a polypeptide, or (2) other than a protein or a polypeptide (e.g., ncRNA, such as siRNA, piRNA, short hairpin RNA or shRNA, microRNA or miRNA or precursors thereof including pre-miRNA and pri-miRNA, antisense sequence or oligonucleotide (ASO), guide RNA or gRNA for CRISPR/Cas, rRNA, tRNA, snoRNA, snRNA, exRNA, scaRNA, lncRNA, Xist, and HOTAIR, etc.).

The ribonucleotide coding sequence for the gene of interest may be further processed inside the cell, once the RNA content of the RAAV viral particle is separated from the AAV capsid and released into the cell. Processing of the coding sequence can produce one or more RNA products, such as siRNA, miRNA, and/or mRNA, which may be further translated into protein product(s), or be incorporated into other cellular machinery such as the RISC complex or a CRISPR/Cas effector enzyme (such as a Class 2, type II, V, or VI effector enzyme).

As used herein, the term “transcribed,” and grammatical variations thereof, refers to a nucleotide sequence comprising ribonucleic acid (RNA) nucleotides that have been transcribed from a DNA template (e.g., double-stranded DNA and/or single-stranded DNA). The transcribed RNA molecule can corresponds to either a plus strand or a minus strand of an AAV ssDNA, wherein the transcribed plus strand RNA was transcribed from the minus strand of the DNA template and the transcribed minus strand RNA was transcribed from the plus strand of the DNA template. In certain embodiments, the transcribed RNA molecule can either be transcribed from the sense or antisense strand of a double stranded DNA template. For example, when the dsDNA sequence is represented by the sequence of only one strand (such as SEQ ID NO: 1), a transcribed RNA using the dsDNA as template may have the same sequence as the sense strand or the antisense strand, as the case may be. That is, RNA transcribed from double-stranded DNA shown as SEQ ID NO: 1 may have the same sequence as SEQ ID NO: 1 or its reverse complement, except that the T's in DNA are replaced by U's in the transcribed RNA.

The transcribed modified AAV inverted terminal repeat (ITR) sequence of the disclosure is an RNA sequence (as opposed to the single-stranded DNA sequence in the conventional AAV viral genome encapsidated within the AAV viral particle). As the wild-type AAV ITR DNA sequence, the transcribed modified AAV ITR sequence (RNA) also supports binding of the RNA of the disclosure to the AAV Rep protein, and is thus capable of supporting the direct packaging of the RNA of the disclosure into the AAV viral particle. In certain embodiments, the transcribed modified ITR sequence comprises a transcribed Rep-binding element (RBE) (e.g., a transcribed functional RBE), and optionally a transcribed RBE′ (e.g., a transcribed functional RBE′), for Rep binding. In certain embodiments, the transcribed modified ITR sequence supports or facilitates packaging or encapsidation of the RNA sequence into an AAV viral particle.

In certain embodiments, the modified ITR comprises a wild-type RBE.

In certain embodiments, the modified ITR comprises a functional RBE that retains at least about 60%, 70%, 80%, 90%, 95%, 100% or more of the ability of wild-type RBE for supporting AAV packaging, such as Rep binding. In certain embodiments, the functional RBE comprises up to about 30%, 25%, 20%, 15%, 10%, or 5% of sequence variation compared to the wild-type RBE, due to, for example, insertion, deletion, substitution, and/or other mutation of one or more nucleotides of the RBE.

In certain embodiments, the modified AAV ITR DNA template, from which the transcribed modified AAV ITR is transcribed, is defective as an ITR, in that it lacks one or more functions of the corresponding wild-type AAV ITR, such as being able to be cleaved at the TRS (transcribed terminal resolution site, see below).

This can be due to, for example, the lack of a functional TRS. In one embodiment, the wild-type TRS is completely deleted such that the modified ITR has no TRS. In one embodiment, the wild-type TRS is mutated by deleting, inserting, substituting, and/or mutating one or more nucleotides such that it can no longer to recognized and cleaved by Rep during AAV replication.

In certain embodiments, the modified AAV ITR DNA template retains the RBE or a functional variant thereof as described herein, and optionally the RBE′ or a functional variant thereof. In certain embodiments, the RBE and/or RBE′ is/are functional with respect to binding to AAV Rep78/68.

The transcribed modified AAV inverted terminal repeat (ITR) of the disclosure further lacks either a transcribed terminal resolution site (TRS), or a transcribed reverse complement TRS (rcTRS), or both. In certain embodiments, the TRS is at the 5′ end of the modified AAV ITR. In certain embodiments, the TRS is between the D region sequence and the RBE.

In certain embodiments, the transcribed modified AAV ITR lacks both the transcribed TRS and the transcribed rcTRS.

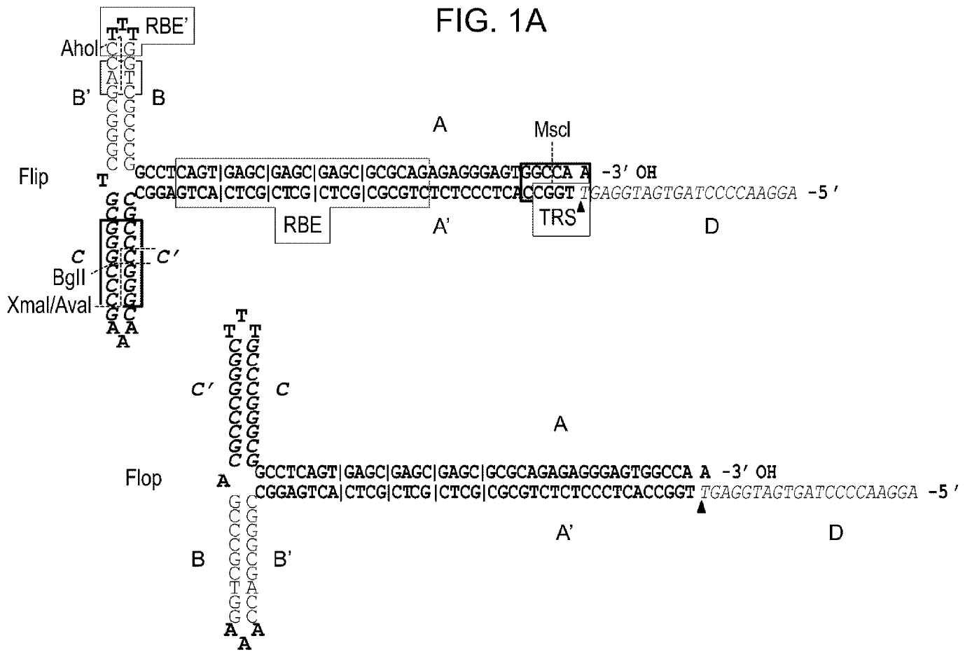

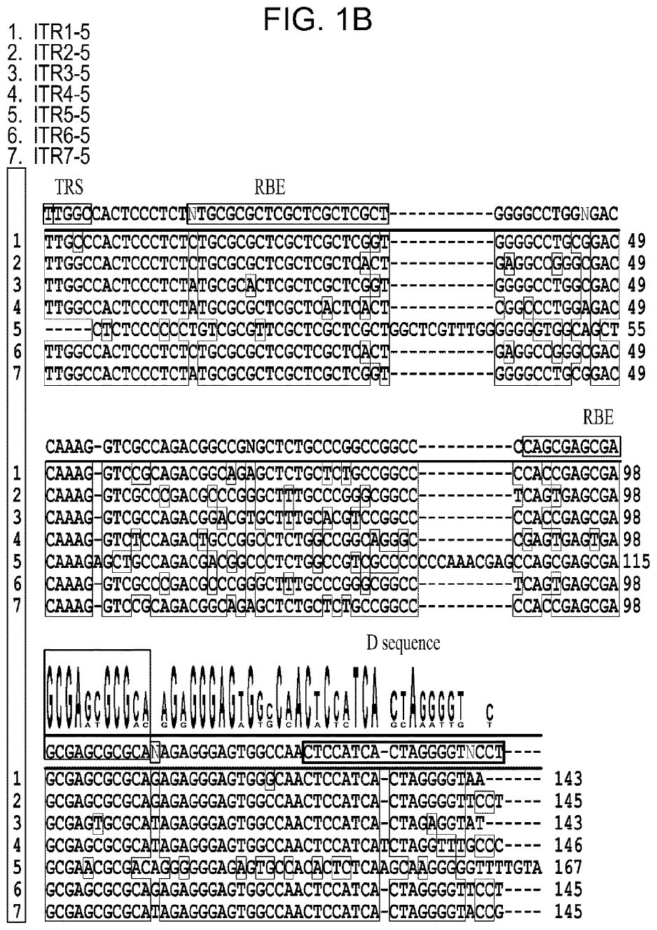

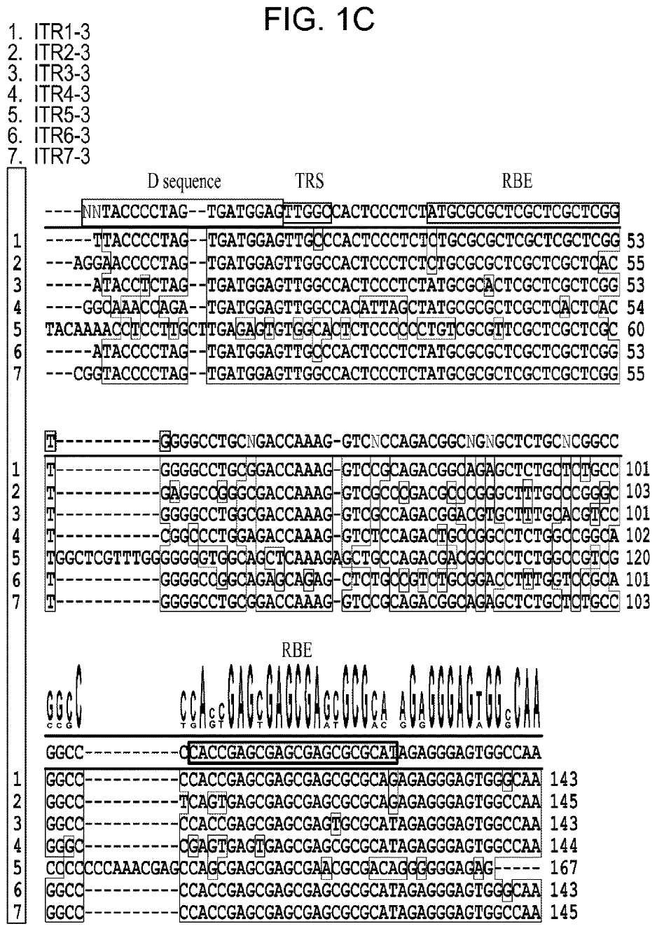

As used herein, “terminal resolution site” or “TRS” refers to the single-stranded DNA sequence in the single-stranded AAV vector genome (plus or minus strand) that is recognized and nicked by the AAV Rep proteins during AAV replication. As used herein, “reverse complement TRS (rcTRS)” refers to the single-stranded DNA sequence in the single-stranded AAV vector genome (plus or minus strand) that is reverse complement sequence of the TRS. The rcTRS pairs with the TRS to form a double stranded DNA region at one end of the A region stem. See FIG. 1A-1C.

In AAV2 ITR, the TRS comprises the sequence of TTGGC, with the Rep cleavage site in between the two T's; while the rcTRS comprises the sequence of GCCAA. One TRS is located at the juncture of the D and A region sequences, and is at the most 5′ end of the A region sequence (e.g., between the D region sequence and the RBE). See FIGS. 1B and 1C for the TRS and rcTRS in 5′ and 3′ ITR multi-sequence alignment of representative AAV's.

As used herein, a “transcribed TRS” is a single-stranded RNA sequence resulting from transcribing the TRS DNA template. For AAV2 TRS comprising TTGGC, the transcribed TRS comprises GCCAA.

As used herein, a “transcribed rcTRS” is a single-stranded RNA sequence resulting from transcribing the rcTRS DNA template. For AAV2 rcTRS comprising GCCAA, the transcribed rcTRS comprises UUGGC.

Thus, a transcribed modified AAV ITR “lacks a transcribed AAV2 TRS,” if it does not have the GCCAA sequence at the location the GCCAA sequence normally appears in a corresponding transcribed wild-type AAV2 ITR, e.g., due to complete deletion of the GCCAA sequence, or due to insertion, deletion, substitution, and/or other mutation of one or more nucleotides within the GCCAA sequence. This can result from transcribing a modified AAV ITR having a complete deletion of the TRS (TTGGC), or due to insertion, deletion, substitution, and/or other mutation of one or more nucleotides within the wild-type TRS.

Thus, in certain embodiments, the RNA of the disclosure or the transcribed modified AAV ITR lacks a transcribed functional TRS.

Similarly, a transcribed modified AAV ITR “lacks a transcribed AAV2 rcTRS,” if it does not have the UUGGC sequence at the location the UUGGC sequence normally appears in a corresponding transcribed wild-type AAV2 ITR, e.g., due to complete deletion of the GCCAA sequence, or due to insertion, deletion, substitution, and/or other mutation of one or more nucleotides within the GCCAA sequence. This can result from transcribing a modified AAV ITR having a complete deletion of the rcTRS, or due to insertion, deletion, substitution, and/or other mutation of one or more nucleotides within the wild-type rcTRS.

In certain embodiments, the transcribed modified AAV ITR further comprises a transcribed D region sequence (D or D′ sequence in a wild-type AAV ITR) or a mutant D region sequence (e.g., one with one or more nucleotide insertion, deletion, substitution, and/or other mutation) that substantially retains the function of a wild-type D region sequence. In other embodiment, the transcribed modified AAV ITR does not comprises a transcribed D region sequence, or does not comprise a mutant D region sequence (e.g., one with one or more nucleotide insertion, deletion, substitution, and/or other mutation) that substantially retains the function of a wild-type D region sequence.

In certain embodiments, the transcribed modified AAV ITR comprises the transcribed (functional) D region sequence. Optionally, the modified AAV ITR DNA template has the nucleotide sequence of SEQ ID NO: 3. Optionally, the transcribed modified AAV ITR comprises an RNA equivalent of SEQ ID NO: 3 (i.e., the RNA equivalent has the same base sequence as the DNA sequence of SEQ ID NO: 3). Optionally, the transcribed modified AAV ITR comprises an RNA equivalent of the reverse complement of SEQ ID NO: 3 (i.e., the RNA equivalent has the same base sequence as the DNA sequence of the reverse complement of SEQ ID NO: 3).

In certain embodiments, the transcribed modified AAV ITR lacks the transcribed (functional) D region sequence. Optionally, the modified AAV ITR DNA template has the nucleotide sequence of SEQ ID NO: 2. Optionally, the transcribed modified AAV ITR comprises an RNA equivalent of SEQ ID NO: 2 (i.e., the RNA equivalent has the same base sequence as the DNA sequence of SEQ ID NO: 2). Optionally, the transcribed modified AAV ITR comprises an RNA equivalent of the reverse complement of SEQ ID NO: 2 (i.e., the RNA equivalent has the same base sequence as the DNA sequence of the reverse complement of SEQ ID NO: 2).

As used herein, “D region sequence” refers to either the D sequence or its reverse complement D′ sequence. Location of the D region sequence depends on whether the ITR takes the “flip” or the “flop” configuration. See FIG. 1A-1C. For example, in wild-type AAV2 ITR (see FIG. 2 of Srivastava et al., J. Viol. 45(2):555-564, 1983, incorporated herein by reference), the plus strand ssDNA sequence comprises, from 5′ to 3′, palindromic sequence segments named A, B, B′, C, C′, A′, D, . . . , D′, A, C, C, B, B′, and A′, in which A:A′, B:B′, C:C′ and D:D′ are reverse complement sequences of each other and can form base-paired stem sequences (though the D and D′ sequences may not actually base-pair with each other in the ssDNA AAV vector genome). The 5′ ITR of the plus strand has the B:B′ stem closer to one end (5′ end) of the sequence than the C:C′ stem, and is known as the flip ITR. The 3′ ITR of the plus strand has the C:C′ stem closer to one end (3′ end) of the sequence than the B:B′ stem, and is known as the flop ITR.

The transcribed modified AAV ITR sequence of the disclosure may lack a functional transcribed D region sequence (D or D′ sequence) by, for example, deletion, insertion, substitution, and/or other mutation of one or more nucleotides of the transcribed wild-type D region sequence.

In certain embodiments, the RNA or transcribed modified AAV ITR sequence of the disclosure comprises a mutated transcribed D region sequence and/or a mutated transcribed TRS sequence. In certain embodiments, the RNA or transcribed modified AAV ITR sequence of the disclosure comprises no transcribed D region sequence and/or no transcribed TRS/rcTRS sequence.

In certain embodiments, the transcribed modified AAV ITR is modified based on a transcribed wild-type flip ITR or a wild-type flop ITR.

In certain embodiments, the wild-type flip ITR or the wild-type flop ITR is from AAV1, AAV2, AAV3A, AAV3B, AAV4, AAV5, AAV6, AAV7, AAV8, AAV9, AAV10, AAV 11, AAV 12, AAV 13, AAVrh10, AAVrh74, AAVhu32, AAVhu37, AAV PHP.eB, Anc80L65, Anc80L65AAP, AAVrh74, or 7m8. Optionally, the wild-type flop ITR has the nucleotide sequence of SEQ ID NO: 1.

In certain embodiments, the transcribed D region sequence is present, and is not within the 3′ end 50 nucleotides (e.g., 40 nt, 30 nt, 25 nt, or 20 nt) of the RNA.

In certain embodiments, the transcribed D region sequence is present, and is within the 3′ end 50 nucleotides (e.g., 40 nt, 30 nt, 25 nt, or 20 nt) of the RNA.

In certain embodiments, the transcribed modified AAV ITR is within the 3′ end 1000 nucleotides of the RNA. In certain embodiments, the transcribed modified AAV ITR is within the 3′ end 800 nucleotides of the RNA.

In certain embodiments, the transcribed modified AAV ITR is within the 3′ end 500 nucleotides of the RNA. In certain embodiments, the transcribed modified AAV ITR is within the 3′ end 300 nucleotides of the RNA. In certain embodiments, the transcribed modified AAV ITR is within the 3′ end 200 nucleotides of the RNA.

In certain embodiments, the transcribed modified AAV ITR is 5′ to a polyA sequence, a polyA signal sequence (e.g., AAUAAA), or a sequence for RNA transcription termination (e.g., a histone downstream element).

As used herein, “polyA sequence” or “polyA tail” refers to a string of adenine ribonucleotides or adenosine monophosphates (e.g., a string of RNA with each base therewithin an adenine). Such a polyA tail is important for the nuclear export, translation and stability of mRNA. The length of the polyA sequence can vary in different mRNA or the RNA of the disclosure, and can be about 250 nucleotides of polyA, about 230 nucleotides of polyA, about 200 nucleotides of polyA, about 180 nucleotides of polyA, about 160 nucleotides of polyA, about 140 nucleotides of polyA, about 120 nucleotides of polyA, about 100 nucleotides of polyA, or less.

As used herein, “polyA signal sequence” refers to an RNA sequence (such as AAUAAA) that is located downstream of the most 3′ exon, and is recognized by an RNA cleavage complex that cleaves off the 3′ terminal sequence of a newly transcribed RNA by RNA polymerase (such as Pol II) such that polyadenylation can occur. Polyadenylate polymerase then adds and extends the poly(A) tail by adding adenosine monophosphate units from ATP to the nascent cleaved 3′ end of the RNA. The initial RNA cleavage is typically catalyzed by the enzyme CPSF (cleavage/polyadenylation specificity factor), and occurs about 10-30 nucleotides downstream of its binding site—the polyA signal sequence, which is often AAUAAA on the transcribed RNA. The sequence at/or immediately 5′ to the site of RNA cleavage is frequently (but not always) CA. The polyA signal sequence recognized by the RNA cleavage complex varies between different groups of eukaryotes, with most human polyadenylation sites containing the AAUAAA sequence, though this sequence is less common in plants and fungi mRNA. In addition, other variants that bind more weakly to CPSF exist. All such sequence motifs recognized by the RNA cleavage complex to enable RNA cleavage and the subsequent polyadenylation are within the scope of the polyA signal sequence.

Also as used herein, “a transcribed GU-rich region downstream of the polyA site” refers to a sequence that may be used by other proteins (such as the cleavage stimulation factor or CstF) to enhance binding specificity of CPSF to the polyA signal sequence (e.g., AAUAAA).

In certain embodiments, the RNA of the disclosure further comprises a recognition sequence for CFI (cleavage factor I), such as a set of UGUAA sequences in mammals, that can recruit CPSF even if the AAUAAA polyA signal sequence is missing.

As used herein, “a sequence for RNA transcription termination” includes an RNA sequence motif present at or near the 3′ end of a transcribed RNA (such as a transcribed RNA without a polyA tail) that terminates transcription. Almost all eukaryotic mRNAs are polyadenylated, with the exception of metazoan replication-dependent histone mRNAs, in which mRNA processing occurs at a site of highly conserved stem-loop structure and a purine rich region around 20 nucleotides downstream. These are the few (if not the only) eukaryotic mRNAs that lack a poly(A) tail, ending instead in a stem-loop structure followed by a purine-rich sequence, termed histone downstream element (HDE) or histone 3′ UTR stem-loop.

HDE directs where the RNA is cleaved during/after transcription, so that the 3′ end of the histone mRNA is formed. HDE is involved in nucleocytoplasmic transport of the histone mRNAs, and in the regulation of stability and of translation efficiency in the cytoplasm.

In certain embodiments, the RNA of the disclosure further comprises a second transcribed modified AAV ITR of the disclosure. In certain embodiments, the second transcribed modified AAV ITR has a transcribed functional RBE sequence but lacks either a second transcribed TRS or a second transcribed rcTRS or both; optionally, the second transcribed modified AAV ITR further comprises or lacks a second transcribed D region sequence. In certain embodiments, the second transcribed modified AAV ITR comprises a second transcribed mutated D region sequence and/or a second transcribed mutated TRS sequence.

In certain embodiments, for the RNA of the disclosure having two transcribed modified AAV ITR, the transcribed modified AAV ITR and the second transcribed modified AAV ITR are identical.

In certain embodiments, for the RNA of the disclosure having two transcribed modified AAV ITR, the transcribed modified AAV ITR and the second transcribed modified AAV ITR are different.

In certain embodiments, the transcribed modified AAV ITR, the second transcribed modified AAV ITR (if present), comprise a deletion from, a mutation in, or an insertion into a corresponding transcribed wild-type AAV ITR D region sequence or a corresponding transcribed wild-type TRS/rcTRS.

In certain embodiments, for the RNA of the disclosure having two transcribed modified AAV ITR, the second transcribed modified AAV ITR is within 5′ end 1000 nucleotides, 800 nucleotides, 500 nucleotides, 250 nucleotides, or 150 nucleotides of the RNA sequence.

In certain embodiments, the RPS comprises an MS2 sequence, an PP7 binding site, or a com binding site, and the RPS-interacting molecule comprises an RPS-interacting protein (RPSIP; e.g., a RPS-binding protein) capably of interacting, e.g., recognizing and binding, directly or indirectly, to the RPS, such as a bacteriophage-derived MS2 coat protein (MCP) for an MS2 sequence, a PP7 bacteriophage coat protein (PCP) for an PP7 binding site, or a phage COM protein (COM) for a com binding site. Sequences of these RPS/RPSIP pair are described in the sequence section of the specification.

Any of the one or more RPS sequences described herein above, including any of the transcribed modified ITR sequences, and any of the MS2 sequence, PP7 binding site, and/or com binding site, alone or in combination, can facilitate the packaging of the RNA of the disclosure into the DNA virus viral particle, in the presence of a suitable/compatible cognate RPSIP.

In certain embodiments, the RPSIP is, or is associated directly or indirectly with, a protein component of the viral packaging system for the DNA virus viral particle. For example, in some embodiments, the RPSIP is a protein component of the viral packaging system for the DNA virus, such as, Rep78, Rep68, Rep52, and/or Rep40 for AAV. For example, in some embodiments, the RPSIP may be directly fused to a protein component of the viral packaging system for the DNA virus. Exemplary protein components of the viral packaging system for AAV include any of the Rep proteins (such as Rep78 and/or Rep68 of adeno-associated virus 2 (AAV2)), and/or any of the assembly-activating protein (AAP).

In certain embodiments, the fusion is an N-terminal fusion wherein the RPSIP (such as MCP, PCP, or COM) is fused N-terminal to a Rep68/78 protein, and/or to an AAP.

In certain embodiments, the fusion is an N-terminal fusion wherein the RPSIP (such as MCP, PCP, or COM) is fused C-terminal to a Rep68/78 protein, and/or to an AAP.

In certain embodiments, the fusion is a direct fusion with no linker sequences in-between.

In certain embodiments, the fusion is through one or more linker sequence, such as a flexible peptide linker that may include a Gly and Ser rich linker or GS linker. Representative GS linkers include 1, 2, 3, 4, 5 or more repeats of Gly or Ser, such as GS, GSS, GSSS (SEQ ID NO: 44), GSSSS (SEQ ID NO: 45), and repeats thereof (e.g., (GSₚ)ₙ (SEQ ID NO: 87), wherein p is an integer between 1-5, and n is an integer between 1-20. One typical such GS linker is GS₃ (SEQ ID NO: 44) linker or GS₄ (SEQ ID NO: 45) linker. In certain embodiments, p is 3 or 4, and n is 1.

In certain embodiments, the RNA of the disclosure can comprise, but preferably does not comprise, a transcribed DNA packaging signal, for example, a transcribed wild-type AAV ITR sequence. For example, the RNA of the disclosure may comprise a transcribed modified AAV ITR sequence having an addition, a deletion, and/or a substitution of a nucleotide of a corresponding transcribed wild-type AAV ITR sequence to reduce the DNA packaging capability of the DNA virus viral particle.

In certain embodiments, the RNA of the disclosure further comprises one or more of: (1) a coding sequence for a protein (such as an mRNA encoding a therapeutic protein or a CRISPR/Cas effector enzyme including any of the Cas effectors described herein below, e.g., Cas9, or a variant thereof, optionally fused to a base editor), a non-coding RNA (ncRNA), or a functional RNA (such as a tRNA, a ribosomal RNA (rRNA), an RNAi reagent or precursor thereof, siRNA, shRNA, miRNA or precursors thereof including pre-miRNA and pri-miRNA, antisense RNA (ASO), piRNA, an RNA component of CRISPR-Cas system such as a guide RNA (or gRNA), a single guide RNA (or sgRNA, chimeric RNA, RNA chimera), a CRISPR RNA (crRNA), or a tracr RNA), snoRNA, snRNA, exRNA, scaRNA, lncRNA, Xist, and HOTAIR, etc.); (2) a transcribed transcription enhancer; (3) a transcribed intron sequence or exon sequence (such as one for enhancing protein expression); (4) a 5′ UTR sequence; (5) a 3′ UTR sequence; (6) a polyA sequence, or a (transcribed) polyadenylation (polyA) signal sequence, and optionally a transcribed polyA site and a transcribed GU-rich region downstream of the polyA site; (7) a posttranscriptional regulatory element or sequence, such as a transcribed Woodchuck Hepatitis Virus (WHP) Posttranscriptional Regulatory Element (WPRE) sequence; and/or, (8) a transcription termination sequence (such as a histone downstream element).

In certain embodiments, the RNA of the disclosure comprises an RPS located 3′ to the posttranscriptional regulatory element or sequence, and 5′ to the polyA sequence or the polyA signal sequence.

For example, in certain embodiments, the RNA of the disclosure comprises, in 5′ to 3′ orientation, the RSI; the optional transcribed WPRE sequence (that may or may not be present); the RPS (such as the transcribed modified AAV ITR, the MS2 sequence, the PP7 binding site, or the com binding site); and the polyA sequence or the polyA signal sequence.

In certain embodiments, the RNA of the disclosure encodes, or the GOI comprises, a protein (e.g., a fluorescent protein, a therapeutic protein, an antigen protein, or a gene-editing protein such as a Cas protein, a ZFN protein, a TALEN protein), an enzyme (such as a Cre protein, or a CRISPR/Cas effector enzyme, e.g., Cas9, Cas12, Cas13, or a variant thereof), a structural protein, an mRNA, a non-coding RNA (ncRNA), an siRNA, a piRNA, a short hairpin RNA or shRNA, a microRNA (miRNA) or a precursor thereof (including pre-miRNA and pri-miRNA), a ribosomal RNA (rRNA), an antisense sequence or oligonucleotide (ASO), an RNA component of a CRISPR-Cas system, including a guide RNA (or a gRNA), such as a single guide RNA (or an sgRNA, a chimeric RNA, an RNA chimera), a CRISPR RNA (crRNA), and a tracr RNA, a guide RNA or gRNA for a CRISPR/Cas effector enzyme, an rRNA, a tRNA, a snoRNA, a snRNA, an exRNA, a scaRNA, a lncRNA, a Xist, and a HOTAIR.

The overall length of the RNA of the disclosure depends on the packaging capacity of the AAV viral particle. Most AAV viral particles have a packaging capacity of about 4,700-5,200 nucleotides, but certain AAV viral particles such as AAV5 particles can package up to 8,900 nucleotides.

Thus, in certain embodiments, the RNA of the disclosure to be packaged into an AAV viral particle is a single-stranded RNA (ssRNA) less than about 8,900 nucleotides in length.

In certain embodiments, the RNA sequence is a ssRNA less than about 8,000 nucleotides in length. In certain embodiments, the RNA sequence is a ssRNA less than about 7,000 nucleotides in length. In certain embodiments, the RNA sequence is a ssRNA less than about 6,000 nucleotides in length. In certain embodiments, the RNA sequence is a ssRNA less than about 5,200 nucleotides in length. In certain embodiments, the RNA sequence is a ssRNA less than about 4,000 nucleotides in length. In certain embodiments, the RNA sequence is a ssRNA less than about 3,000 nucleotides in length. In certain embodiments, the RNA sequence is a ssRNA less than about 2,000 nucleotides in length.

In certain embodiments, the RNA sequence is a ssRNA about 4,700-5,200 nucleotides in length. In certain embodiments, the RNA sequence is a ssRNA about 4,700-5,000 nucleotide in length. In certain embodiments, the RNA sequence is a ssRNA about 4,700-4,800 nucleotides in length. In certain embodiments, the RNA sequence is a ssRNA about 4,700 nucleotides in length.

Another aspect of the disclosure provides a polynucleotide comprising a (transcription) cassette encoding the RNA of the disclosure; optionally, the polynucleotide is a DNA sequence (e.g., a DNA plasmid), optionally comprising a stuffer sequence in the backbone of the DNA plasmid, and/or optionally comprising no functional DNA packaging signal such as AAV ITR.

In certain embodiments, the polynucleotide comprising the cassette is a DNA vector encoding the RNA of the disclosure. Such DNA vector and/or the cassette thereof can be used to transcribe and produce the RNA of the disclosure for further packaging into, e.g., an AAV viral particle.

In certain embodiments, the polynucleotide further comprises a promoter operably linked to and driving the transcription of the RNA of the disclosure encoded by the cassette to produce the RNA of the disclosure.

In certain embodiments, the promoter is a ubiquitous promoter.

In certain embodiments, the promoter is a tissue-specific promoter.

In certain embodiments, the promoter is a constitutive promoter.

In certain embodiments, the promoter is an inducible promoter.

In certain embodiments, the polynucleotide further comprises an enhancer that enhances the transcription of the RNA sequence driven by the promoter.

Another aspect of the disclosure provides a recombinant DNA virus viral particle comprising an RNA genome (such as the RNA of the disclosure, or the RNA sequence transcribed from the polynucleotide of the disclosure) packaged within the protein shell (such as capsid) of a DNA virus (such as an AAV virus, or an oncolytic virus).

In certain embodiments, the DNA virus is AAV, and the recombinant DNA virus viral particle is a recombinant RNA adeno-associated virus (rRAAV) particle, comprising: (1) an AAV capsid; and, (2) the RNA of the disclosure, or the RNA sequence transcribed from the polynucleotide of the disclosure, packaged within the AAV capsid.

In certain embodiments, the AAV capsid comprises a capsid from an AAV of the serotype AAV1, AAV2, AAV3A, AAV3B, AAV4, AAV5, AAV6, AAV7, AAVrh74, AAV8, AAV9, AAV10, AAV11, AAV12, AAV13, AAV-DJ, AAV PHP.eB, Anc80L65, Anc80L65AAP, or 7m8.

A related aspect of the disclosure provides a population of recombinant DNA virus viral particles (e.g., rRAAV particles) comprising a plurality of recombinant DNA virus viral particle (e.g., rRAAV particle) of the disclosure, wherein at least about 10%, 20%, 30%, 40%, 50%, 60%, 70%, 80%, 90%, 95%, 96%, 97%, 98%, 99% or more of the recombinant DNA virus viral particles (e.g., rRAAV particles) within the population have encapsidated RNA sequence of the disclosure, or the RNA sequence transcribed from the polynucleotide of the disclosure packaged therein.

In certain embodiments, the population of recombinant viral particles (e.g., rRAAV particles) comprises at least 1×10⁴ viral particles, at least 2×10⁴ viral particles, at least 5×10⁴ viral particles, at least 1×10⁵ viral particles, at least 2×10⁵ viral particles, at least 5×10⁵ viral particles, at least 1×10⁶ viral particles, at least 2×10⁶ viral particles, at least 5×10⁶ viral particles, at least 1×10⁷ viral particles, at least 2×10⁷ viral particles, at least 5×10⁷ viral particles, at least 1×10⁷ viral particles, at least 2×10⁸ viral particles, at least 5×10⁸ viral particles, at least 1×10⁹ viral particles, at least 2×10⁹ viral particles, at least 5×10⁹ viral particles, at least 1×10¹⁰ viral particles, at least 2×10¹⁰ viral particles, at least 5×10¹⁰ viral particles, at least 1×10¹¹ viral particles, at least 2×10¹¹ viral particles, at least 5×10¹¹ viral particles, at least 1×10¹¹ viral particles, at least 2×10¹¹ viral particles, at least 5×10¹¹ viral particles, at least 1×10¹³ viral particles, at least 2×10¹³ viral particles, at least 5×10¹³ viral particles, at least 1×10¹⁴ viral particles, at least 2×10¹⁴ viral particles, at least 5×10¹⁴ viral particles, at least 1×10¹⁵ viral particles, at least 2×10¹⁵ viral particles, at least 5×10¹⁶ viral particles, at least 1×10¹⁶ viral particles, at least 2×10¹⁶ viral particles, or at least 5×10¹⁶ viral particles.

In certain embodiments, at most 50%, 45%, 40%, 35%, 30%, 25%, 20%, 15%, 10%, 5%, 3%, 2%, 1%, 0.1%, 0.01% or less of the population of recombinant viral particles encapsidate non-RNA (e.g., DNA) within the viral particles.

Another aspect of the disclosure provides a host cell comprising the RNA of the disclosure, the polynucleotide of the disclosure, the RNA sequence transcribed from the polynucleotide of the disclosure, the recombinant DNA virus viral particle (e.g., rRAAV particle) of the disclosure, and/or the population of recombinant DNA virus viral particle (e.g., rAAV particle) of the disclosure.

In certain embodiments, the host cell further comprises a viral packaging system that facilitates packaging of the RNA of the disclosure, or the RNA sequence transcribed from the polynucleotide of the disclosure into the DNA virus viral particle.

In certain embodiments, the viral packaging system comprises: (1) an AAV rep gene (e.g., coding sequence for Rep78, Rep68, Rep52, and/or Rep40) and an AAV cap gene (e.g., coding sequence for VP1, VP2, and/or VP3, AAP, and/or MAAP), under the transcriptional control of one or more promoters that drive the transcription of the rep gene and cap gene, or the expression products thereof; (2) one or more coding sequences for one or more proteins required for AAV packaging, such as adenoviral E2A, E4, and VA genes, or the one or more proteins; and (3) the RPS-interacting molecule or a coding sequence thereof.

In certain embodiments, the capacity of the viral packaging system of packaging a DNA sequence into the DNA virus viral particle is reduced, diminished, or substantially eliminated by, for example, (1) removing a part or all of the DNA packaging signals such as AAV ITR on the polynucleotide encoding the RNA of the disclosure or on the polynucleotide of the disclosure, (2) modifying, e.g., mutating, the AAV rep gene, the AAV cap gene, and/or the one or more coding sequences for one or more proteins required for AAV packaging to reduce, diminish, or substantially eliminate the capacity of the respective translated protein in order to facilitate the packaging of the DNA sequence into the DNA virus viral particle (e.g., a Y156F mutation in the common sequence of Rep78 and Rep68 proteins, KDE-mu, or EKE-mu); and/or (3) enlarging the size of the polynucleotide encoding the RNA of the disclosure or the polynucleotide of the disclosure. In an embodiment, enlarging the size of the polynucleotide encoding the RNA of the disclosure or the polynucleotide of the disclosure is made by inserting a stuffer sequence (e.g., an intron) into (e.g., the backbone of) the polynucleotide (e.g., a DNAplasmid).

In certain embodiments, the AAV rep gene, the AAV cap gene, and/or the proteins required for AAV packaging comprises a mutation that diminishes or reduces capacity to facilitate packaging of DNA into the DNA virus viral particle.

In certain embodiments, the Rep68/Rep 78 protein required for DNA packaging comprises a mutation that compromises or diminishes its trs-endonuclease activity. The trs-endonuclease activity is believed to be required to resolve AAV replication (DNA) intermediates at the trs sequence or site, such that individual units of AAV ssDNA can be resolved before packaging into the AAV capsid.

In certain embodiments, the trs-endonuclease mutation comprise a Y156F mutation in the common sequence of Rep78 and Rep68 proteins.

In certain embodiments, the Rep78/Rep68 proteins comprise a KDE-mu mutation (see sequence below in the sequence section).

In certain embodiments, Rep78/Rep68 proteins comprise a EKE-mu mutation (see sequence below in the sequence section).

In certain embodiments, Rep78/Rep68 proteins comprise two or more mutations selected form the Y156F mutation, the KDE-mu mutation, and the EKE-mu mutation.

In certain embodiments, the Rep68/Rep78 are from any one of the AAVs with serotype of AAV1, AAV2, AAV3A, AAV3B, AAV4, AAV5, AAV6, AAV7, AAV8, AAV9, AAV10, AAV11, AAV12, AAV13, AAV-DJ, AAV PHP.eB, Anc80L65, Anc80L65AAP, AAVrh74, or 7m8, and has a corresponding trs-endonuclease mutation of the Y156F mutation, the KDE-mu mutation, and/or the EKE-mu mutation.

In certain embodiments, the host cell further comprises: (1) a coding sequence for an AAV rep gene and an AAV cap gene, under the transcriptional control of one or more promoters that drive the transcription of the rep gene and cap gene; and, (2) coding sequences for proteins required for AAV packaging, such as adenoviral E2A, E4, and VA genes.

In certain embodiments, the host cell is a mammalian cell, such as a HEK293 cell or a variant thereof (e.g., HEK293T cell), or an insect cell, such as Sf9 or Sf21 cells.

Another aspect of the disclosure provides a method of generating the recombinant DNA virus viral particle (e.g., rRAAV particle) or the population of recombinant DNA virus viral particles (e.g., rRAAV particles) of the disclosure, the method comprising: a) culturing the host cell of the disclosure for a sufficient time, and b) harvesting the recombinant DNA virus viral particle or the population of recombinant DNA virus viral particles.

In certain embodiments, the method further comprises isolating or purifying the recombinant DNA virus viral particle or the population of recombinant DNA virus viral particles.

Another aspect of the disclosure provides a method of generating a recombinant DNA virus viral particle (e.g., rRAAV particle) or a population of recombinant DNA virus viral particles, the method comprising: a) contacting a viral packaging system (e.g., an AAV packaging system) with the RNA of the disclosure or the RNA sequence transcribed from the polynucleotide of the disclosure, for a period of time sufficient to produce the recombinant DNA virus viral particle of the disclosure, or the population of recombinant DNA virus viral particles of the disclosure, and b) harvesting the recombinant DNA virus viral particle of the disclosure, or the population of recombinant DNA virus viral particles of the disclosure; and, optionally, c) isolating or purifying the harvested recombinant DNA virus viral particle of the disclosure, or the population of recombinant DNA virus viral particles of the disclosure.

In certain embodiments, the viral packaging system (e.g., a AAV packaging system) comprises: (1) one or more proteins for assemblying the protein shell (e.g., VP1, VP2, and/or VP3 for assembling AAV capsid) of the DNA virus viral particle for packaging the RNA sequence, or one or more coding sequences thereof; (2) one or more proteins (e.g., Rep78, Rep68, Rep52, and/or Rep40 for AAV packaging) for facilitating the assemblying of the protein shell and/or the packaging of the RNA sequence into the protein shell of the DNA virus viral particle, or one or more coding sequences thereof (e.g., adenoviral E2a, E4, and VA genes); and (3) the RPS-interacting molecule or a coding sequence thereof. Optionally, the capacity of the viral packaging system of packaging a DNA sequence into the DNA virus viral particle is reduced, diminished, or substantially eliminated by, for example, (1) removing a part or all of the DNA packaging signals such as AAV ITR on the polynucleotide encoding the RNA of the disclosure, or on the polynucleotide of the disclosure, (2) modifying, e.g., mutating, the AAV rep gene, the AAV cap gene, and/or the one or more coding sequences for one or more proteins required for AAV packaging to reduce, diminish, or substantially eliminate the capacity of the respective translated protein to facilitate the packaging of the DNA sequence into the DNA virus viral particle (e.g., a Y156F mutation in the common sequence of Rep78 and Rep68 proteins, KDE-mu, or EKE-mu); and/or (3) enlarging the size of the polynucleotide encoding the RNA of the disclosure or the polynucleotide of the disclosure.

Another aspect of the disclosure provides a system of packaging the RNA of the disclosure or the RNA sequence transcribed from the polynucleotide of the disclosure into a DNA virus viral particle, the system comprising: (1) one or more proteins for assemblying the protein shell (e.g., VP1, VP2, and/or VP3 for assembling AAV capsid) of the DNA virus viral particle for packaging the RNA sequence, or one or more coding sequences thereof; (2) one or more proteins (e.g., Rep78, Rep68, Rep52, and/or Rep40 for AAV packaging) for facilitating the assemblying of the protein shell and/or the packaging of the RNA of the disclosure into the protein shell of the DNA virus viral particle, or one or more coding sequences thereof (e.g., adenoviral E2a, E4, and VA genes); and (3) the RPS-interacting molecule or a coding sequence thereof. Optionally, the capacity of the viral packaging system of packaging a DNA sequence into the DNA virus viral particle is reduced, diminished, or substantially eliminated by, for example, (1) removing a part or all of the DNA packaging signals such as AAV ITR on the polynucleotide encoding the RNA of the disclosure or on the polynucleotide of the disclosure, (2) modifying, e.g., mutating, the AAV rep gene, the AAV cap gene, and/or the one or more coding sequences for one or more proteins required for AAV packaging to reduce, diminish, or substantially eliminate the capacity of the respective translated protein to facilitate the packaging of the DNA sequence into the DNA virus viral particle (e.g., a Y156F mutation in the common sequence of Rep78 and Rep68 proteins, KDE-mu, or EKE-mu); and/or (3) enlarging the size of the polynucleotide encoding the RNA of the disclosure or the polynucleotide of the disclosure.

Another aspect of the disclosure provides a method of delivering an RNA sequence of interest (RSI) into a cell, a plant, or an animal, the method comprising contacting the cell, the plant, or the animal with the recombinant DNA virus viral particle (e.g., rRAAV particle) of the disclosure, the population of recombinant DNA virus viral particles (e.g., rRAAV particles) of the disclosure, or the recombinant DNA virus viral particle (e.g., rRAAV particle) or the population of recombinant DNA virus viral particles (e.g., rRAAV particles) produced by the method of the disclosure, wherein the GOI is optionally encoded by the RNA of the disclosure.

Another aspect of the disclosure provides a method of diagnosing, preventing, or treating a disease or disorder in a subject in need thereof, comprising administrating to the subject a therapeutically effective amount or dose of the population of the recombinant DNA virus viral particles (e.g., rRAAV particles) of the disclosure, or produced by the method of the disclosure.

Another aspect of the disclosure provides a use of the recombinant DNA virus viral particle (e.g., rRAAV particle) of the disclosure, the population of the recombinant DNA virus viral particles (e.g., rRAAV particles) of the disclosure, or the recombinant DNA virus viral particle (e.g., rRAAV particle) or the population of the recombinant DNA virus viral particles (e.g., rRAAV particles) produced by the method of the disclosure, in the manufacture of a medicament for diagnosing, preventing, or treating a disease or disorder in a subject in need thereof.

Another aspect of the disclosure provides a fusion protein or a conjugate, comprising an RPSIP of the disclosure fused or conjugated to a protein component of the viral packaging system for the DNA virus, wherein the RPSIP interacts with/binds to an RPS on the RNA of the disclosure to facilitate the packaging of the RNA sequence into the DNA virus.

In certain embodiments, the RPS is MS2, and the RPSIP is MCP.

In certain embodiments, the RPS is PP7 binding site, and the RPSIP is PCP.

In certain embodiments, the RPS is com, and the RPSIP is phage COM protein.

In certain embodiments, the fusion or conjugate comprises more than one RPSIP, each independently binds to one or more RPS on the RNA of the disclosure. In certain embodiments, at least two of the more than one RPSIP are identical. In certain embodiments, at least two of the more than one RPSIP are different.

In certain embodiments, the fusion or conjugate comprises two MCP in tandem.

In certain embodiments, the protein component of the viral packaging system for the DNA virus comprises a Rep protein of an AAV, such as a Rep68 or a Rep78 of the AAV.

In certain embodiments, the Rep protein comprises one or more mutations that compromises or diminishes trs-endonuclease activity. In certain embodiments, the mutations comprise the Y156F mutation, the KDE-mu mutation, and/or the EKE-mu mutation.

In certain embodiments, the protein component of the viral packaging system for the DNA virus comprises an assembly-activating protein (AAP).

In certain embodiments, the RPSIP is fused to the protein component of the viral packaging system for the DNA virus (e.g., a Rep protein or an AAP) directly.

In certain embodiments, the RPSIP is fused to the protein component of the viral packaging system for the DNA virus (e.g., a Rep protein or an AAP) through a peptide linker.

In certain embodiments, the peptide linker is a flexible linker, such as a Gly and Ser containing linker. In certain embodiments, the Gly and Ser containing linker comprises 1-20 repeats (e.g., 1-5 or 1-3 repeats) of GSₙ, wherein n is 1, 2, 3, 4, or 5. In certain embodiments, the GSₙ linker is GS₂, GS₃ (SEQ ID NO: 44), or GS₄ (SEQ ID NO: 45), with 1-4 (e.g., 2) repeats. In certain embodiments, the linker is GSSGSS (SEQ ID NO: 46).

In certain embodiments, the fusion protein comprises MCP and Rep, wherein the Rep optionally comprises a Y156F mutation, a KDE-mu mutation, and/or a EKE-mu mutation. In certain embodiments, MCP is fused N-terminal to Rep (MCP-Rep). In certain embodiments, the Rep fused to MCP comprises a Y156F mutation, a KDE-mu mutation, and/or a EKE-mu mutation. In certain embodiments, the MCP-Rep fusion is linked by a GSₙ linker, such as GSSGSS (SEQ ID NO: 46). In certain embodiments, the MCP-Rep comprises two MCP in tandem (e.g., without any linker between the two MCP moieties). In certain embodiments, the MCP is C-terminal to another GSₙ linker, such as GSSGSS (SEQ ID NO: 46).

In certain embodiments, the fusion protein comprises PCP and Rep, wherein the Rep optionally comprises a Y156F mutation, a KDE-mu mutation, and/or a EKE-mu mutation. In certain embodiments, PCP is fused N-terminal to Rep (PCP-Rep). In certain embodiments, the Rep fused to PCP comprises a Y156F mutation, a KDE-mu mutation, and/or a EKE-mu mutation. In certain embodiments, the PCP-Rep fusion is linked by a GSₙ linker, such as GSSGSS (SEQ ID NO: 46). In certain embodiments, the PCP-Rep comprises two PCP in tandem (e.g., without any linker between the two PCP moieties). In certain embodiments, the PCP is C-terminal to another GSₙ linker, such as GSSGSS (SEQ ID NO: 46).

In certain embodiments, the fusion protein comprises COM and Rep, wherein the Rep optionally comprises a Y156F mutation, a KDE-mu mutation, and/or a EKE-mu mutation. In certain embodiments, COM is fused N-terminal to Rep (COM-Rep). In certain embodiments, the Rep fused to COM comprises a Y156F mutation, a KDE-mu mutation, and/or a EKE-mu mutation. In certain embodiments, the COM-Rep fusion is linked by a GSₙ linker, such as GSSGSS (SEQ ID NO: 46). In certain embodiments, the COM-Rep comprises two COM in tandem (e.g., without any linker between the two COM moieties). In certain embodiments, the COM is C-terminal to another GSₙ linker, such as GSSGSS (SEQ ID NO: 46).

In certain embodiments, the fusion protein comprises MCP and AAP. In certain embodiments, MCP is fused N-terminal to AAP (MCP-AAP, or MA). In certain embodiments, MCP is fused C-terminal to AAP (AAP-MCP, or AM). In certain embodiments, the MCP-AAP or AAP-MCP fusion is linked by a GSₙ linker, such as GSSGSS (SEQ ID NO: 46). In certain embodiments, the MCP-AAP fusion is C-terminal to another GSₙ linker, such as GSSGSS (SEQ ID NO: 46). In certain embodiments, the AAP-MCP fusion is N-terminal to another GSₙ linker, such as GSSGSS (SEQ ID NO: 46).

Another aspect of the disclosure provides a polynucleotide encoding any one of the fusions between the RPSIP of the disclosure and the protein component of the viral packaging system for the DNA virus (e.g., AAP or a Rep protein).

With the general aspects of the disclosure described herein above, the following sections provides additional details for specific elements of the disclosure described herein. Each specific element is contemplated to be able to combined with any one or more additional elements of the disclosure, even though all possible combinations or permutations of the elements are not explicitly recited.

### 2. AAV Serotypes

AAV particles packaging ribopolynucleotides of the disclosure may comprise or be derived from any natural or recombinant AAV serotypes.

According to the disclosure, the AAV particles may utilize or be based on a serotype selected from any of the following serotypes, and variants thereof, including but not limited to: AAV1, AAV10, AAV106.1/hu.37, AAV11, AAV114.3/hu.40, AAV 12, AAV127.2/hu.41, AAV127.5/hu.42, AAV128.1/hu.43, AAV128.3/hu.44, AAV130.4/hu.48, AAV145.1/hu.53, AAV145.5/hu.54, AAV145.6/hu.55, AAV16.12/hu.11, AAV16.3, AAV16.8/hu.10, AAV161.10/hu.60, AAV161.6/hu.61, AAVl-7/rh.48, AAVl-8/rh.49, AAV2, AAV2.5T, AAV2-15/rh.62, AAV223.1, AAV223.2, AAV223.4, AAV 223.5, AAV223.6, AAV223.7, AAV2-3/rh.61, AAV24.1, AAV2-4/rh.50, AAV2-5/rh.51, AAV27.3, AAV29.3/bb.1, AAV29.5/bb.2, AAV2G9, AAV-2-pre-miRNA-101, AAV3, AAV3.1/hu.6, AAV3.1/hu.9, AAV3-11/rh.53, AAV3-3, AAV33.12/hu. 17, AAV33.4/hu. 15, AAV33.8/hu.16, AAV3-9/rh.52, AAV3a, AAV3b, AAV4, AAV4-19/rh.55, AAV42.12, AAV42-10, AAV42-11, AAV42-12, AAV42-13, AAV42-15, AAV42-1b, AAV42-2, AAV42-3a, AAV42-3b, AAV42-4, AAV42-5a, AAV42-5b, AAV42-6b, AAV42-8, AAV42-aa, AAV43-1, AAV43-12, AAV43-20, AAV43-21, AAV43-23, AAV43-25, AAV43-5, AAV4-4, AAV44.1, AAV44.2, AAV44.5, AAV46.2/hu.28, AAV46.6/hu.29, AAV4-8/r 11.64, AAV4-8/rh.64, AAV4-9/rh.54, AAV5, AAV52.1/hu.20, AAV52/hu.19, AAV5-22/rh.58, AAV5-3/rh.57, AAV54.1/hu.21, AAV54.2/hu.22, AAV54.4R/hu.27, AAV54.5/hu.23, AAV54.7/hu.24, AAV58.2/hu.25, AAV6, AAV6.1, AAV6.1.2, AAV6.2, AAV7, AAV7.2, AAV7.3/hu.7, AAV8, AAV-8b, AAV-8h, AAV9, AAV9.11, AAV9.13, AAV9.16, AAV9.24, AAV9.45, AAV9.47, AAV9.61, AAV9.68, AAV9.84, AAV9.9, AAV A3.3, AAV A3.4, AAV A3.5, AAV A3.7, AAV-b, AAVC1, AAVC2, AAVC5, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAV-DJ, AAV-DJ8, AAVF3, AAVF5, AAV-h, AAVH-1/hu.1, AAVH2, AAVH-5/hu.3, AAVH6, AAVhE1.1, AAVhER1.14, AAVhEr1.16, AAVhEr1.18, AAVhER1.23, AAVhEr1.35, AAVhEr1.36, AAVhEr1.5, AAVhEr1.7, AAVhEr1.8, AAVhEr2.16, AAVhEr2.29, AAVhEr2.30, AAVhEr2.31, AAVhEr2.36, AAVhEr2.4, AAVhEr3.1, AAVhu.1, AAVhu.10, AAVhu.11, AAVhu.1, AAVhu.12, AAVhu.13, AAVhu.14/9, AAVhu.15, AAVhu.16, AAVhu.17, AAVhu.18, AAVhu.19, AAVhu.2, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.3, AAVhu.31, AAVhu.32, AAVhu.34, AAVhu.35, AAVhu.37, AAVhu.39, AAVhu.4, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.5, AAVhu.51, AAVhu.52, AAVhu.53, AAVhu.54, AAVhu.55, AAVhu.56, AAVhu.57, AAVhu.58, AAVhu.6, AAVhu.60, AAVhu.61, AAVhu.63, AAVhu.64, AAVhu.66, AAVhu.67, AAVhu.7, AAVhu.8, AAVhu.9, AAVhu.t 19, AAVLG-10/rh.40, AAVLG-4/rh.38, AAVLG-9/hu.39, AAVLG-9/hu.39, AAV-LK01, AAV-LK02, AAVLK03, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LKt5, AAV-LK17, AAV-LK18, AAV-LK19, AAVN721-8/rh.43, AAV-PAEC, AAV-PAEC11, AAV-PAEC12, AAV-PAEC2, AAV-PAEC4, AAV-PAEC6, AAV-PAEC7, AAV-PAEC8, AAVpi. 1, AAVpi.2, AAVpi.3, AAVrh.10, AAVrh.12, AAVrh.13, AAVrh. 13R, AAVrh.14, AAVrh.17, AAVrh.18, AAVrh.19, AAVrh.2, AAVrh.20, AAVrh.21, AAVrh.22, AAVrh.23, AAVrh.24, AAVrh.25, AAVrh.2R, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.43, AAVrh.44, AAVrh.45, AAVrh.46, AAVrh.47, AAVrh.48, AAVrh.48, AAVrh.48.1, AAVrh.48.1.2, AAVrh.48.2, AAVrh.49, AAVrh.50, AAVrh.51, AAVrh.52, AAVrh.53, AAVrh.54, AAVrh.55, AAVrh.56, AAVrh.57, AAVrh.58, AAVrh.59, AAVrh.60, AAVrh.61, AAVrh.62, AAVrh.64, AAVrh.64R1, AAVrh.64R2, AAVrh.65, AAVrh.67, AAVrh.68, AAVrh.69, AAVrh.70, AAVrh.72, AAVrh.73, AAVrh.74, AAVrh.8, AAVrh.8R, AAVrh8R, AAVrh8R A586R mutant, AAVrh8R R533A mutant, BAAV, BNP6t AAV, BNP62 AAV, BNP63 AAV, bovine AAV, caprine AAV, Japanese AAV 10, true type AAV (ttAAV), UPENN AAV 10, AAV-LK16, AAAV, AAV Shuffle 100-1, AAV Shuffle 100-2, AAV Shuffle 100-3, AAV Shuffle 100-7, AAV Shuffle 10-2, AAV Shuffle 10-6, AAV Shuffle 10-8, AAV SM 100-10, AAV SM 100-3, AAV SM 10-1, AAV SM 10-2, and/or AAV SM 10-8.

In certain embodiments, the AAV serotype may comprise a mutation in the AAV9 sequence, such as the sequence described by Pulicherla et al. (Molecular Therapy 19(6): 1070-1078, 2011), such as AAV9.9, AAV9.11, AAV9.13, AAV9.16, AAV9.24, AAV9.45, AAV9.47, AAV9.61, AAV9.68, AAV9.84.

In certain embodiments, the AAV serotype may comprise a sequence described in U.S. Pat. No. 6,156,303, such as AAV3B (SEQ ID NOs: 1 and 10 of U.S. Pat. No. 6,156,303), AAV6 (SEQ ID NOs: 2, 7 and 11 of U.S. Pat. No. 6,156,303), AAV2 (SEQ ID NOs: 3 and 8 of U.S. Pat. No. 6,156,303), AAV3A (SEQ ID NOs: 4 and 9, of U.S. Pat. No. 6,156,303), or derivatives thereof.

In certain embodiments, the serotype may be AAV-DJ or a variant thereof, such as AAVDJ8 (or AAV-DJ8), as described by Grimm et al. (Journal of Virology 82(12): 5887-5911, 2008). The amino acid sequence of AAV-DJ8 may comprise two or more mutations in order to remove the heparin binding domain (HBD).

As a non-limiting example, the AAV-DJ sequence described as SEQ ID NO: 1 in U.S. Pat. No. 7,588,772 may comprise two mutations: (1) R587Q (Arg at amino acid 587 is changed to glutamine Gln), and (2) R590T.

As another non-limiting example, the AAV-DJ sequence may comprise three mutations: (1) K406R, (2) R587Q, and (3) R590T.

In certain embodiments, the AAV serotype may comprise a sequence as described in WO2015/121501, such as true type AAV (ttAAV) (SEQ ID NO: 2 of WO2015/121501), the so-called UPenn AAV10 (SEQ ID NO: 8 of WO2015/121501), or the so-called Japanese AAV10 (SEQ ID NO: 9 of WO2015/121501), or variants thereof.

AAV capsid serotype selection or use may be from a variety of species. In certain embodiments, the AAV may be an avian AAV (aAAV). The aAAV serotype may comprise a sequence described in U.S. Pat. No. 9,238,800, such as aAAV (SEQ ID NOs: 1, 2, 4, 6, 8, 10, 12, and 14 of U.S. Pat. No. 9,238,800), or variants thereof.

In certain embodiments, the AAV may be a bovine AAV (bAAV). The bAAV serotype may comprise a sequence described in U.S. Pat. No. 9,193,769, such as bAAV (SEQ ID NOs: 1 and 6 of U.S. Pat. No. 9,193,769), or variants thereof.

The bAAV serotype may comprise a sequence as described in U.S. Pat. No. 7,427,396, such as bAAV (SEQ ID NOs: 5 and 6 of U.S. Pat. No. 7,427,396), or variants thereof.

In certain embodiments, the AAV may be a caprine AAV. The caprine AAV serotype may comprise a sequence described in U.S. Pat. No. 7,427,396, such as caprine AAV (SEQ ID NO: 3 of U.S. Pat. No. 7,427,396), or variants thereof.

In certain embodiments, the AAV may be engineered as a hybrid AAV from two or more parental serotypes.

In certain embodiments, the AAV may be AAV2G9, which comprises sequences from AAV2 and AAV9. The AAV2G9 AAV serotype may comprise a sequence described in US 2016-0017005 A1. (incorporated herein by reference).

In certain embodiments, the AAV may be a serotype generated by the AAV9 capsid library with mutations in amino acids 390-627 (VP1 numbering) as described by Pulicherla et al. (Molecular Therapy 19(6): 1070-1078, 2011, incorporated herein by reference). The serotype and corresponding nucleotide and amino acid substitutions may be, but is not limited to: AAV9.1 (G1594C; D532H), AAV6.2 (T1418A and T1436X; V473D and 1479K), AAV9.3 (T1238A; F413Y), AAV9.4 (T1250C and A1617T; F417S), AAV9.5 (A1235G, A1314T, A1642G, C1760T; Q412R, T548A, A587V), AAV9.6 (T1231A; F4111), AAV9.9 (G1203A, G1785T, W595C), AAV9.10 (A1500G, T1676C; M559T), AAV9.11 (A1425T, A1702C, A1769T; T568P, Q590L), AAV9.13 (A1369C, A1720T; N457H, T574S), AAV9.14 (T1340A, T1362C, T1560C, G1713A; L447H), AAV9.16 (A1775T; Q592L), AAV9.24 (T1507C, T1521G; W503R), AAV9.26 (A1337G, A1769C; Y446C, Q590P), AAV9.33 (A1667C; D556A), AAV9.34 (A1534G, C1794T; N512D), AAV9.35 (A1289T, T1450A, C1494T, A1515T, C1794A, G1816A; Q430L, Y484N, N98K, V6061), AAV9.40 (A1694T, E565V), AAV9.41 (A1348T, T1362C; T450S), AAV9.44 (A1684C, A1701T, A1737G; N562H, K567N), AAV9.45 (A1492T, C1804T; N498Y, L602F), AAV9.46 (G1441C, T1525C, T1549G; G481R, W509R, L517V), 9.47 (G1241A, G1358A, A1669G, C1745T; S414N, G453D, K557E, T5821), AAV9.48 (C1445T, A1736T; P482L, Q579L), AAV9.50 (A1638T, C1683T, T1805A; Q546H, L602H), AAV9.53 (G1301A, A1405C, C1664T, G1811T; R134Q, S469R, A555V, G604V), AAV9.54 (C1531A, T1609A; L511I, L537M), AAV9.55 (T1605A; F535L), AAV9.58 (C1475T, C1579A; T492I, H527N), AAV.59 (T1336C; Y446H), AAV9.61 (A1493T; N4981), AAV9.64 (C1531A, A1617T; L511I), AAV9.65 (C1335T, T1530C, C1568A; A523D), AAV9.68 (C1510A; P504T), AAV9.80 (G1441A; G48IR), AAV9.83 (C1402A, A1500T; P468T, E500D), AAV9.87 (T1464C, T1468C; S490P), AAV9.90 (A1196T; Y399F), AAV9.91 (T1316G, A1583T, C1782G, T1806C; L439R, K528I), AAV9.93 (A1273G, A1421G, A1638C, C1712T, G1732A, A1744T, A1832T; S425G, Q474R, Q546H, P571L, G578R, T582S, D611V),AAV9.94 (A1675T; M559L) and AAV9.95 (T1605A; F535L).

In certain embodiments, the AAV may be a serotype comprising at least one AAV capsid CD8+ T-cell epitope. As a non-limiting example, the serotype may be AAV1, AAV2 or AAV 8.

In certain embodiments, the AAV may be a variant, such as PHP.A or PHP.B as described in Deverman (Nature Biotechnology. 34(2): 204-209, 2016, incorporated herein by reference).

In certain embodiments, the AAV may be a serotype generated by Cre-recombination-based AAV targeted evolution (CREATE) described by Deverman et al., (Nature Biotechnology 34(2):204-209, 2016, incorporated herein by reference). In certain embodiments, the AAV serotypes generated in this manner have improved CNS transduction and/or neuronal and astrocytic tropism, as compared to other AAV serotypes.

In some embodiments, the AAV serotypes may be an AAV9 derivative with a 7-amino acid insertion between amino acids 588-589. Non-limiting examples of these 7-amino acid insertions include PHP.A, PHP.B, PHP.B2, PHP.B3, PHP.N, PHP.S, G2A12, G2A15, G2A3, G2B4, and G2B5.

In certain embodiments, the AAV may be a serotype selected from any of those found in SEQ ID NOs: 4,734-5,302 and in Table 2 of WO2018/002719A1 (incorporated herein by reference). In certain embodiments, the AAV may be encoded by a sequence, fragment or variant as described in SEQ ID NOs: 4,734-5,302 of WO2018/002719A1 (incorporated herein by reference).

In certain embodiments, the AAV VP1 capsid sequence is one of the following: AAV1, AAV2, AAV3A, AAV3B, AAV4, AAV5, AAV6, AAV7, AAV8, AAV9, AAV10, AAV11, AAV12, AAV13, AAV-DJ, AAV PHP.eB, Anc80L65, Anc80L65AAP, or 7m8.

### 3. Modified AAV ITR

Any transcribed AAV ITR sequences (RNA) can be modified according to the disclosure herein, by engineering the encoding modified AAV ITR DNA template to, e.g., eliminate or inactivate the TRS or equivalent thereof, and/or to eliminate the D region sequence thereof. The transcribed modified AAV ITR, resulting from transcribing such modified AAV ITR DNA template, retains the ability to facilitate the packaging of the RNA of the disclosure into an AAV viral particle.

During AAV DNA replication, the ITRs are nicked by the virus-encoded Rep proteins at the terminal resolution site (TRS). This origin function requires three DNA sequence elements, namely the Rep binding element (RBE), a small palindrome that comprises a single tip of an internal hairpin within the terminal repeat (RBE′), and the TRS. Rep is tethered to the RBE (DNA) in a specific orientation during TRS nicking. This orientation appears to align Rep on the AAV ITR, allowing specific nucleotide contacts with the RBE′ and directing nicking to the TRS. Alterations in the polarity or position of the RBE relative to the TRS greatly inhibit Rep nicking. Substitutions within the RBE′ also reduce Rep specific activity, but only to a lesser extent. Rep interactions with the RBE and RBE′ during TRS nicking are functionally distinct, in that the Rep contact with the RBE is necessary for both the DNA helicase activity and the TRS cleavage. Meanwhile, Rep interaction with RBE′ is required primarily for ITR unwinding and formation of the TRS stem-loop structure, but is not required for TRS cleavage.

At least one transcribed modified ITR sequence (RNA) of the disclosure is present on the RNA of the disclosure. The transcribed modified ITR sequence of the disclosure is preferably located closer to the 3′ end of the RNA of the disclosure.

In certain embodiments, the RNA of the disclosure comprises two transcribed modified ITR sequences.

In certain embodiments, the two transcribed modified ITR sequences may be derived from the same AAV serotype.

In another embodiment, the two transcribed modified ITR sequences may be derived from two different AAVs of different serotypes.

In certain embodiments, the transcribed modified ITR sequence(s) include(s) an insertion, deletion, and/or a mutation.

In some embodiments, the rRAAV RNA sequence of the disclosure comprises one transcribed modified/mutated ITR sequence and one transcribed wild-type ITR sequence.

In some embodiments, the transcribed modified ITR sequence(s) is/are based on a wild-type ITR in either the flip orientation or the flop orientation.

The subject transcribed modified ITR sequences, or their coding DNA sequences, can be readily prepared based on wild-type ITR sequences known in the art.

Representative (non-limiting) wild-type ITR sequences (DNA) including at least the following sequences listed in Table 1. A multi-sequence alignment for the 5′ ITR sequences, and a multi-sequence alignment for the 3′ ITR sequences of AAV1-AAV7 are shown in FIGS. 1B and 1C, respectively, including the consensus sequences, the TRS, the RBE, and the D region sequences.

As used herein, “RBE sequence” or “RBE” refers to the AAV ITR sequences within the A:A′ palindromic stem sequences that, when base-paired, form a stem (usually a double stranded region of about 21-23, or about 22 bp) and facilitate ITR binding to AAV Rep proteins (Rep78 and Rep68). A representative RBE sequence is shown in FIG. 1A, in both the flip and flop configurations of the wild-type AAV2 ITR.

Wild-type ITR sequences of the numerous AAV serotypes known in the art are readily available, each can be aligned with the other AAV ITRs as in FIGS. 1B and 1C. The results of the alignment can be used to identify the RBE sequences for any AAV ITR.

A “transcribed (functional) RBE” refers to a transcribed RNA corresponding to the RBE DNA template, which is either wild-type RBE, or a functional variant thereof with one or more nucleotide insertions, deletions, substitutions, and/or other mutations, so long as the functional variant RBE substantially retains the ability to bind to Rep (e.g., retains at least about 60%, 70%, 80%, 90%, 95%, or enhanced binding to Rep of the same serotype). In certain embodiments, the RBE DNA template or the transcribed RBE RNA differs from the wild-type sequence by no more than 10, 9, 8, 7, 6, 5, 4, 3, 2, 1 nucleotide(s). In certain embodiments, the functional RBE comprises up to about 30%, 25%, 20%, 15%, 10%, or 5% of sequence variation compared to the wild-type RBE, due to, for example, insertion, deletion, substitution, and/or other mutation of one or more nucleotides of the RBE.

In certain embodiments, the nucleotide sequence difference does not result in loss of paired base pair (e.g., a GC pair in the wild-type RBE can be changed to CG, AT/AU or TA/UA in the variant RBE without losing the original paired base pair).

In certain embodiments, the transcribed modified ITR sequence (RNA) retains a transcribed Rep-binding element (transcribed RBE) or a functional variant thereof, to facilitate Rep-mediated packaging. For example, the RBE DNA sequence for wild-type AAV2 ITR is SEQ ID NO: 5.

In certain embodiments, the transcribed modified ITR sequence (RNA) further retains a transcribed Rep-binding element′ (transcribed RBE′) sequence. For example, in FIG. 1A, the CTTTG DNA sequence forming the hairpin or loop structure in the B:B′ segment of the flip ITR is the RBE′ sequence.

In certain embodiments, the transcribed modified ITR sequence lacks a transcribed TRS, and/or a transcribed rcTRS, or both.

In certain embodiments, the RNA of the disclosure lacks a transcribed (functional) TRS sequence, due to the fact that its corresponding DNA sequence lacks certain sequence elements of the wild-type TRS, such that wild-type TRS function is lost in the DNA (e.g., the sequence or internal strand normally occupied by the wild-type TRS sequence between the A:A′ segment and D region sequence, which is normally recognized and cleaved by endonuclease during AAV replication, is not cleaved if present in the ssDNA vector genome of AAV ITR).

For example, in some embodiments, the reverse complement of the TRS may be deleted or mutated, as in the dITR and dITR-D sequence used in the examples.

Alternatively or in addition, the TRS normally between the A:A′ segment and D region sequence may lack one or more nucleotides, or have one or more nucleotide substitutions or mutations (such as lacking or substituting/mutating 4 nucleotides in the dITR sequence used in the examples).

In certain embodiments, the entire or nearly the entire TRS/rcTRS in the wild-type sequence is deleted such that the resulting RNA transcript lacks a functional TRS sequence.

In certain embodiments, a part of the wild-type TRS/rcTRS sequence is altered/mutated by, for example, having an insertion, deletion, substitution, and/or other mutation in the wild-type sequence, such that the mutated TRS/rcTRS produces a corresponding RNA transcript that lacks a transcribed functional TRS. For example, in certain embodiments, 1 2, 3, 4, or 5 consecutive or non-consecutive TRS nucleotides and/or rcTRS nucleotides can be deleted or substituted in a mutated sequence.

In certain embodiments, the transcribed modified ITR sequence is transcribed from a modified ITR lacking a D region sequence, or at least a functional D region sequence (D sequence or D′ sequence, depending on the flip or flop configuration). For example, in some embodiments, the entire D region sequence is deleted such that the resulting RNA transcript lacks a transcribed functional D region sequence. In other embodiments, at least a portion of the D region sequence is mutated (e.g., having deletion, insertion, substitution, and/or other mutation) such that the resulting RNA transcript lacks a transcribed functional D region sequence. In certain embodiments, the mutated D region sequence has no more than 10, 9, 8, 7, 6, 5, 4, 3, 2, or 1 nucleotide of the wild-type sequence.

In certain embodiments, the modified ITR sequence (DNA template) lacks 1, 2, 3, 4, 5, 6, 7, 8, 9, or 10 most 5′ end nucleotides of the wild-type ITR sequence. For example, the dITR sequence (SEQ ID NO: 2) and the dITR-D sequence (SEQ ID NO: 3) both lack the most 5′ end 8 nucleotides compared to the wild-type ITR sequence (SEQ ID NO: 1).

Corresponding DNA sequences encoding any of the above described transcribed RNA coding sequence (DNA coding sequence for the GOI), transcribed modified AAV ITR (modified AAV ITR), transcribed functional RBE (functional RBE), transcribed functional D region sequence (functional D region sequence), and transcribed functional TRS sequence (functional TRS sequence) are expressly contemplated as within the scope of the disclosure.

### 4. Introns, Exons, UTRs, Enhancers, and Other Elements

The RNA sequence of the disclosure to be encapsidated in the rRAAV viral particles of the disclosure may further comprise additional optional sequence elements (such as expression control elements) that may enhance or regulate the expression of the GOI.

Expression control elements present within the RNA of the disclosure facilitate proper heterologous polynucleotide (e.g., GOI) transcription and/or translation, including, e.g., splicing signal for introns, maintenance of the correct reading frame of the gene to permit in-frame translation of mRNA and, stop codons etc.

Typically, expression control elements, some within the RNA of the disclosure, and others present on the DNA encoding the RNA of the disclosure, are nucleic acid sequence(s), such as promoters and enhancers that influence expression of an operably linked heterologous polynucleotide (e.g., GOI). Such elements typically act in cis but may also act in trans. Expression control can be effected at the level of transcription, translation, splicing, message stability, etc. Typically, an expression control element that modulates transcription is juxtaposed near the 5′ end (i.e., “upstream”) of the transcribed polynucleotide. Expression control elements can also be located at the 3′ end (i.e., “downstream”) of the transcribed sequence or within the transcript (e.g., in an intron). Expression control elements can be located at a distance away from the transcribed gene of interest sequence (e.g., 100 to 500, 500 to 1000, 2,000 to 5,000, or more nucleotides from the gene of interest polynucleotide). Nevertheless, owing to the polynucleotide length limitations for viral vectors, such as AAV vectors, such expression control elements will typically be within 1-1,000, 1-500, 1-250, or 1-100 nucleotides from the transcribed gene of interest sequence.

Some non-limiting expression control elements that may be present on the RNA of the disclosure, or DNA encoding the RNA of the disclosure, are described in further details herein below.

### Introns

Introns are known to possess a posttranscriptional regulatory element that efficiently induces transport of mRNA out of the nucleus and enhances mRNA stability.

In certain embodiments, the rRAAV can include one or more introns or a fragment thereof. In some embodiments, the one or more introns are fragments of the gene of interest. In some embodiments, the one or more introns are heterologous to the gene of interest.

Introns have been reported to affect the levels of gene expression. This effect is known as Intron Mediated Enhancement (IME) of gene expression (Lu et al., Mol Genet Genomics 279:563-572, 2008). In some embodiments, the levels of gene expression are increases by about 1.5-fold, about 2-fold, about 2.5-fold, about 3-fold, about 3.5 fold, about 4-fold, about 4.5-fold, about 5-fold, about 5.5-fold, about 6-fold, about 6.5-fold, about 7-fold, about 7.5-fold, about 8-fold, about 8.5-fold, about 9-fold, about 9.5-fold, or about 10-fold when compared to gene expression from a sequence without the one or more introns.

Non-limiting introns include SV40 intron, beta globin intron, and short chimeric intron (CIB). Other introns include the ColE2-RNA-OUT, OIPR, and R6K-RNA-OUT introns described in Lu et al., Hum Gene Ther. 2017; 28(1):125-134 (incorporated by reference); the human hemoglobin subunit beta (HBB2) synthetic intron (Snyder et al., Hum Gene Ther, 8 (1997), pp. 1891-1900, incorporated by reference).

In some embodiments, the one or more introns may be less than 25 nucleotides, less than 50 nucleotides, less than 100 nucleotides, less than 150 nucleotides, less than 200 nucleotides, less than 250 nucleotides, less than 300 nucleotides, less than 350 nucleotides, less than 400 nucleotides, less than 450 nucleotides, or less than 500 nucleotides.

In some embodiments, the one or more introns may be more than 25 nucleotides, more than 50 nucleotides, more than 100 nucleotides, more than 150 nucleotides, more than 200 nucleotides, more than 250 nucleotides, more than 300 nucleotides, more than 350 nucleotides, more than 400 nucleotides, more than 450 nucleotides, or more than 500 nucleotides.

In some embodiments, the one or more introns may be about 50 to about 100 nucleotides, about 50 to about 200 nucleotides, about 50 to about 300 nucleotides, about 50 to about 400 nucleotides, about 50 to about 500 nucleotides, about 100 to about 200 nucleotides, about 100 to about 300 nucleotides, about 100 to about 400 nucleotides, about 100 to about 500 nucleotides, about 200 to about 300 nucleotides, about 200 to about 400 nucleotides, about 200 to about 500 nucleotides, about 300 to about 400 nucleotides, about 300 to about 500 nucleotides, or about 400 to about 500 nucleotides.

### Enhancers

The term “enhancer” as used herein can refer to a sequence that is located adjacent to the gene of interest. Enhancer elements are typically located upstream of a promoter element in the DNA encoding the RNA of the disclosure, but can also be located downstream of or within an intron sequence (e.g., a gene of interest) and remain functional. Thus the enhancer or part thereof may be present in the transcribed RNA sequence of the disclosure.

Non-limiting examples of suitable enhancers include a CMV enhancer.

In certain embodiments, an enhancer element can be located 100 base pairs, 200 base pairs, or 300 or more base pairs upstream or downstream of a gene of interest (e.g., in the RNA of the disclosure or a DNA coding sequence therefor). Enhancer elements typically increase expressed of a gene of interest above increased expression afforded by a promoter element.

### Untranslated Regions (UTRs)

As used herein, “Untranslated Regions” (“UTRs”) refer to RNA that are not translated after transcription. For example, the 5′ UTR is upstream of the start code of the gene of interest and the 3′ UTR is downstream of the stop codon of the gene of interest. In some embodiments, the 5′ and/or 3′ UTRs may have an insertion, deletion, or modification to enhance stability of the transcribed gene of interest. For Example, the 5′ UTR may comprise a translation initiation sequence such as, but not limited to, a Kozak sequence and an internal ribosome entry site (IRES). Kozak sequences have the consensus CCR(A/G)CCAUGG (SEQ ID NO: 47), where R is a purine (adenine or guanine) three bases upstream of the start codon (AUG), which is followed by another ‘G’.

3′ UTRs are known to have stretches of Adenosines and Uridines embedded in them. These AU rich signatures are particularly prevalent in genes with high rates of turnover. Based on their sequence features and functional properties, the AU rich elements (AREs) can be separated into three classes (Chen et al, 1995): Class I AREs contain several dispersed copies of an AUUUA motif within U-rich regions. C-Myc and MyoD contain class I AREs. Class II AREs possess two or more overlapping UUAUUUA(U/A)(U/A) nonamers. Molecules containing this type of AREs include GM-CSF and TNF-a. Class III ARES are less well defined. These U rich regions do not contain an AUUUA motif. c-Jun and Myogenin are two well-studied examples of this class. Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo. Any of these 5′ and/or 3′ UTR sequences can be present in the RNA of the disclosure.

In some embodiments, the 5′ UTR and/or 3′UTR may comprise heterologous sequence to the gene of interest. In some embodiments, the 5′ UTR and/or 3′ UTR are native to the gene of interest.

In certain embodiments, a 5′ UTR and/or a 3′ UTR from an mRNA normally expressed in a specific tissue or organ, such as lung, liver, pancreas, endothelial cells, CNS, neurons, astrocytes, skeletal muscle, cardiac muscle, smooth muscle, blood, hematopoietic cells may be used in the RNA of the disclosure comprising a GOT targeted to one or more of these tissues.

### Polyadenylation Sequence

In certain embodiments, the RNA of the disclosure comprise a transcribed modified AAV ITR that is 5′ to a polyA sequence, a polyA signal sequence (e.g., AAUAAA), or a sequence for RNA transcription termination (e.g., a histone downstream element).

The “polyA sequence,” “polyA tail,” “polyA signal sequence,” and “a sequence for RNA transcription termination” are defined herein above.

In certain embodiments, the RNA of the disclosure comprises a polyA tail. Such RNA sequence can be packaged into the rRAAV viral particles of the disclosure and be delivered directly into a target cell, and the GOT encoded by the RNA of the disclosure can be directly translated.

In certain embodiments, the RNA of the disclosure comprises a polyA signal sequence and optionally a transcribed GU-rich region downstream of the polyA site. Such RNA sequence can be packaged into the rRAAV viral particles of the disclosure and be delivered directly into a target cell. Once inside the target cell, the polyA signal sequence may be recognized and further processed by the cytosolic polyA addition enzymes to produce a polyA tail, before the GOT encoded by the RNA of the disclosure is translated.

Representative polyA signal sequence and surrounding sequences include human growth hormone (hGH) polyA sequence (see Liu et al., Gene Ther 20:308-317, 2013, incorporated by reference), bovine growth hormone polyadenylation signal (bGHpA) (Goodwin and Rottman, J Biol Chem. 1992 Aug. 15; 267(23):16330-4, incorporated by reference), SV40 early or late polyadenylation signal, and the synthetic polyA signal used in Choi et al. (Mol Brain. 2014; 7:17, incorporated herein by reference).

### Transcription Enhancer

As used herein, a “transcription enhancer” refer to cis-acting nucleotide sequences that can increase the transcription of the gene of interest. In some embodiments, the transcription enhancer can be located in the intron or partially in an exon region of the transcribed RAAV RNA sequence of the disclosure.

### WPRE

In certain embodiments, the RNA of the disclosure comprises a transcribed WPRE sequence, encoded by the WPRE sequence on the encoding DNA.

Woodchuck Hepatitis Virus (WHP) Posttranscriptional Regulatory Element (WPRE) is a 600-bp or so DNA sequence that, when transcribed, creates a tertiary structure enhancing expression.

WPRE is commonly used in molecular biology to increase expression of genes delivered by viral vectors. It is a tripartite regulatory element with gamma, alpha, and beta components. The alpha component is 80 bp long: GCCACGGCGGAACTCATCGCCGCCTGCCTTGCCCGCTGCTGGACAGGGGCTCGGCTGTTGG GCACTGACAATTCCGTGGT (SEQ ID NO: 39). When used alone, the alpha component is only 9% as active as the full tripartite WPRE sequence, which is 100% identical to base pairs 1093-1684 of the Woodchuck hepatitis B virus (WHV8) genome.

In certain embodiments, the transcribed WPRE sequence or part thereof (such as the gamma, alpha, and beta elements, preferably in the given order) is present in a 3′ UTR region of a GOI on the subject RNA sequence encapsidated in the rRAAV viral particle of the disclosure, to substantially increase stability and protein yield of the RNA of the disclosure.

In certain embodiments, the WPRE sequence is a shorted WPRE (WPRE2) containing a minimal gamma element and a partial alpha-beta element (see Kalev-Zylinska, J Neurosci. 2007, 27: 10456-10467, incorporated by reference).

In certain embodiments, the WPRE sequence is a shorted WPRE (WPRE3) containing minimal gamma and alpha elements (see Choi et al., Mol Brain 7, 17 (2014), incorporated by reference).

In certain embodiments, the RNA of the disclosure comprises a WPRE sequence and a GOI lacking introns.

### Promoters

The term “promoter” as used herein is defined as a DNA sequence recognized by the synthetic machinery of the cell, or introduced synthetic machinery, required to initiate the specific transcription of a polynucleotide sequence.

Thus the RNA of the disclosure does not comprise a promoter. On the other hand, a DNA encoding the RNA of the disclosure (such as an expression cassette or expression vector encoding the RNA of the disclosure) comprises a promoter for transcribing the RNA of the disclosure.

As used herein, the term “promoter/regulatory sequence” means a nucleic acid sequence which is required for expression of a gene product operably linked to the promoter/regulatory sequence. In some instances, this sequence may be the core promoter sequence. In other instances, this sequence may also include an enhancer sequence and other regulatory elements which are required for expression of the gene product.

The promoter/regulatory sequence may, for example, be one which expresses the gene product (e.g., the RNA of the disclosure) in a tissue or cell type specific manner.

As used herein, the term “operable linkage” or “operably linked” refers to a physical or functional juxtaposition of the components so described as to permit them to function in their intended manner. In the example of an expression control element in operable linkage with a heterologous polynucleotide, the relationship is such that the control element modulates expression of the heterologous polynucleotide. More specifically, for example, two DNA sequences operably linked means that the two DNAs are arranged (cis or trans) in such a relationship that at least one of the DNA sequences is able to exert a physiological effect upon the other sequence.

In certain embodiments, the promoter is a constitutive promoter.

As used herein, a “constitutive” promoter is a nucleotide sequence which, when operably linked with a polynucleotide which encodes or specifies a gene product, causes the gene product to be produced in a cell under most or all physiological conditions of the cell.

In certain embodiments, a promoter that can be used to constitutively drive the expression of the RNA of the disclosure from a DNA encoding the same can include: a p glucuronidase (GUSB) promoter, a cytomegalovirus (CMV) immediate-early (Je) enhancer and/or promoter, a chicken β-actin (CBA) promoter or derivative thereof such as a CAG promoter, CB promoter, a (human) elongation factor 1α-subunit (EF1α) promoter, and a ubiquitin C (UBC) promoter.

In certain embodiments, the promoter is an inducible promoter.

As used herein, an “inducible” promoter is a nucleotide sequence which, when operably linked with a polynucleotide which encodes or specifies a gene product, causes the gene product to be produced in a cell substantially only when an inducer which corresponds to the promoter is present in the cell.

In certain embodiments, the promoter is a tissue-specific promoter, a species specific promoter, or a cell cycle-specific promoter. See Parr et al., Nat. Med. 3:1145-9, 1997 (entire contents incorporated herein by reference).

As used herein, a “tissue- or cell-type-specific” promoter is a nucleotide sequence which, when operably linked with a polynucleotide encodes or specified by a gene, causes the gene product to be produced in a specific cell type or a specific tissue preferentially, due to, for example, the cell/tissue is a cell type or tissue type in which the promoter is normally active.

Tissue- or cell type-specific promoters may include neuronal tissue specific promoter; CNS- or PNS-specific promoter such as astrocyte, oligodendrocyte, or neuronal promotor; hematopietic lineage specific promoter such as B cell promoter, T cell promoter, NK cell promoter, monocyte promoter, leukocyte promoter, macrophage promoter; endothelial cell promoter; pancreatic promoter; liver/hepatic cell promoter; lung tissue promoter, etc.

Representative tissue-specific promoters include prion promoter, neuron-specific enolase (NSE), neurofilament light (NFL) promoter, neurofilament heavy (NFH) promoter, platelet-derived growth factor (PDGF), platelet-derived growth factor B-chain (PDGF-β), synapsin (Syn), synapsin 1 (Syn1), methyl-CpG binding protein 2 (MeCP2), Ca2+/calmodulin-dependent protein kinase II (CaMKII), metabotropic glutamate receptor 2 (mGluR2), neurofilament light (NFL) or heavy (NFH), β-globin minigene nP2, preproenkephalin (PPE), enkephalin (Enk) and excitatory amino acid transporter 2 (EAAT2) promoters.

Astrocyte-specific promoters include glial fibrillary acidic protein (GFAP) and EAAT2 promoters.

Oligodendrocyte-specific promoters include the myelin basic protein (MBP) promoter.

In some embodiments, the promoter is heterologous to the gene of interest. In some embodiments, the promoter is the natural promoter of the gene of interest. In some embodiments, the heterologous promoter includes an insertion, deletion, substitution, and/or other mutation. In some embodiments, the natural promoter includes an insertion, deletion, substitution, and/or other mutation.

In certain embodiments, the promoter is a Pol II promoter. In certain embodiments, the promoter is a Pol III promoter, such as U6 promoter.

### 5. Vectors (Plasmids or Bacmids)

As used herein, a “vector” generally refers to a composition of matter which comprises an isolated nucleic acid (DNA or RNA) and which can be used to deliver the isolated nucleic acid to the interior of a cell.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids, bacmids (e.g., naked or contained in liposomes) and viruses (e.g., lentiviruses, retroviruses, adenoviruses, and adeno-associated viruses) that incorporate the recombinant polynucleotide.

An rRAAV RNA sequence of the disclosure comprising a GOI is a vector for delivering the GOI into a target/host cell through a rRAAV viral particle encapsidating the vector.

In certain embodiments, the rRAAV RNA sequence of the disclosure is encoded by a DNA expression vector, such as a plasmid or bacmid (e.g., one that can be maintained or replicated like a baculovirus inside an insect cell). Such DNA expression vector can transcribe the RNA of the disclosure within a suitable host cell, such as a mammalian packaging cell (e.g., HEK293T cells) or an insect packaging cell (e.g., Sf9 cells), such that the subject rRAAV viral particles can be produced in the presence of other elements necessary for rRAAV packaging (such as rep and cap coding sequences).

Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

In some embodiments, the RAAV is transcribed from a plasmid or bacmid. The plasmid or bacmid can include the gene of interest sequence. In some embodiments, the promoter is operably linked to the gene of interest and is located upstream of the gene of interest. In some embodiments, promoter is not in the transcribed RAAV.

### 6. AAV Particles and Populations of AAV Particles

In certain embodiments, the invention provides an isolated rRAAV viral particle comprising any one of the RNA of the disclosure encapsidated within any one of the AAV capsid or viral particle described herein.

In some embodiments, the AAV capsid or viral particle is of a serotype or a combination of one or more serotypes described herein.

In the rRAAV vectors or RNA of the disclosure, the rRAAV genome (RNA) may be either a single stranded (ss) nucleic acid or a double stranded (ds), self-complementary (sc) nucleic acid.

A related aspect of the disclosure provides a population of recombinant viral particles (e.g., rRAAV particles) comprising a plurality of recombinant viral particle (e.g., rRAAV particle) of the disclosure, wherein at least about 10%, 20%, 30%, 40%, 50%, 60%, 70%, 80%, 90%, 95%, 96%, 97%, 98%, 99% or more of the recombinant viral particles (e.g., rRAAV particles) within the population have encapsidated RNA sequence of the disclosure.

In some embodiments, the population of rRAAV particles contain a plurality of rRAAV viral particle of the disclosure, wherein about 40%, 50%, 60%, 70%, 80%, 90%, 95%, 96%, 97%, 98%, 99% or more of the rRAAV particles within the population have encapsidated RNA sequence of the disclosure.

In certain embodiments, the population of recombinant viral particles (e.g., rRAAV particles) comprises at least 1×10⁴ viral particles, at least 2×10⁴ viral particles, at least 5×10⁴ viral particles, at least 1×10⁵ viral particles, at least 2×10⁵ viral particles, at least 5×10⁵ viral particles, at least 1×10⁶ viral particles, at least 2×10⁶ viral particles, at least 5×10⁶ viral particles, at least 1×10⁷ viral particles, at least 2×10⁷ viral particles, at least 5×10⁷ viral particles, at least 1×10⁸ viral particles, at least 2×10⁸ viral particles, at least 5×10⁸ viral particles, at least 1×10⁹ viral particles, at least 2×10⁹ viral particles, at least 5×10⁹ viral particles, at least 1×10¹⁰ viral particles, at least 2×10¹⁰ viral particles, at least 5×10¹⁰ viral particles, at least 1×10¹¹ viral particles, at least 2×10¹¹ viral particles, at least 5×10¹¹ viral particles, at least 1×10¹² viral particles, at least 2×10¹² viral particles, at least 5×10¹² viral particles, at least 1×10¹³ viral particles, at least 2×10¹³ viral particles, at least 5×10¹³ viral particles, at least 1×10¹⁴ viral particles, at least 2×10¹⁴ viral particles, at least 5×10¹⁴ viral particles, at least 1×10¹⁵ viral particles, at least 2×10¹⁵ viral particles, at least 5×10¹⁵ viral particles, at least 1×10¹⁶ viral particles, at least 2×10¹⁶ viral particles, or at least 5×10¹⁶ viral particles.

In certain embodiments, at most 50%, 45%, 40%, 35%, 30%, 25%, 20%, 15%, 10%, 5%, 3%, 2%, 1%, 0.1%, 0.01% or less of the population of recombinant viral particles encapsidate non-RNA (e.g., DNA) within the viral particles.

### 7. Host Cells and AAV Production

General principles of rAAV production are known in the art. See review in, for example, Carter (Current Opinions in Biotechnology, 1533-539, 1992); and Muzyczka, Cur: Topics in Microbial, and Immunol 158:97-129, 1992, both incorporated herein by reference). Various approaches are described in Ratschin et al (Mol. Cell. Biol. 4:2072, 1984; Hermonat et al. (Proc. Natl. Acad. Sci. USA 81:6466, 1984); Tratschin et al. (Mol. Cell. Biol. 5:3251, 1985); McLaughlin et al. (J. Virol 62:1963, 1988); and Lebkowski et al. (Mol. Cell. Biol 7:349, 1988), Samulski et al. (J Virol 63:3822-3828, 1989); U.S. Pat. No. 5,173,414; WO 95/13365 and U.S. Pat. No. 5,658,776; WO 95/13392; WO 96/17947; PCT/US98/18600; WO 97/09441; WO 97/08298; WO 97/21825; WO 97/06243; WO 99/11764; Perrin et al. (Vaccine 13:1244-1250, 1995; Paul et al. (Human Gene Therapy 4:609-615, 1993); Clark et al. (Gene Therapy 3:1124-1132, 1996; U.S. Pat. Nos. 5,786,211; 5,871,982; and 6,258,595.

AAV vector serotypes can be matched to target cell types. For example, Table 2 of WO2018002719A1 lists exemplary cell types that can be transduced by the indicated AAV serotypes (incorporated herein by reference).

Packaging cells are used to form virus particles that are capable of infecting a host cell. Such cells include HEK293 and Sf9 cells, which can be used to package AAV and adenovirus.

Viral vectors used in gene therapy are usually generated by a producer cell line that packages a nucleic acid vector into a viral particle. The vectors typically contain the minimal viral sequences required for packaging and subsequent integration into a host (if applicable), other viral sequences being replaced by an expression cassette encoding the protein to be expressed. The missing viral functions can be supplied in trans by the packaging cell line, usually as a result of expression of these viral functions/proteins (such as the rep and cap genes for AAV) either as transgenes integrated into the packaging cell, or as transgenes on a second viral vector or expression vector introduced into the packaging cell.

For example, AAV vectors used in gene therapy typically only possess inverted terminal repeat (ITR) sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV.

In some embodiments, recombinant AAVs maybe produced using the triple transfection method (described in detail in U.S. Pat. No. 6,001,650). Typically, the recombinant AAVs are produced by transfecting a host cell with an recombinant AAV vector (comprising a gene of interest) to be packaged into AAV particles, an AAV helper function vector, and an accessory function vector. An AAV helper function vector encodes the “AAV helper function” sequences (e.g., rep and cap), which function in trans for productive AAV replication and encapsidation. Preferably, the AAV helper function vector supports efficient AAV vector production without generating any detectable wild-type AAV virions (e.g., AAV virions containing functional rep and cap genes). The accessory function vector encodes nucleotide sequences for non-AAV derived viral and/or cellular functions upon which AAV is dependent for replication (e.g., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpesvirus (other than herpes simplex virus type-1), and vaccinia virus.

In some embodiments, the subject rRAAV is produced using a baculovirus expression system packaged in insect cells such as Sf9 cells. See, for example, WO2007046703, WO2007148971, WO2009014445, WO2009104964, WO2013036118, WO2011112089, WO2016083560, WO2015137802, and WO2019016349, all incorporated herein by reference.

The vector titers are usually expressed as viral genomes per ml (vg/ml). In certain embodiments, viral titers is above 1×10⁹, above 5×10¹⁰, above 1×10¹¹, above 5×10¹¹, above 1×10¹², above 5×10¹², or above 1×10¹³ vg/ml.

### 8. Gene of Interest (GOI) or RNA Sequence of Interest (RSI)

The rRAAV particles of the disclosure can be used to deliver any gene of interest (GOI) or RNA sequence of interest (RSI) to a host cell, for any purpose, so long as the GOI is an RNA within the packaging limit of the chosen AAV viral capsid or AAV viral particle shell, such as about 4,700 nucleotides overall length for most AAV viral particles, up to about 8,900 nucleotides for certain large capacity AAV viral particles such as AAV5.

In certain embodiments, representative (non-limiting) RNA sequence of interest (RSI) includes, for example, a protein-encoding RNA, an mRNA, a non-coding RNA (ncRNA), a tRNA, a ribosomal RNA (rRNA), a transfer-messenger RNA (tmRNA), an antisense oligonucleotide (ASO), an RNA aptamer, an RNA component of CRISPR-Cas system such as a single guide RNA (or sgRNA, chimeric RNA, RNA chimera), CRISPR RNA (crRNA), tracr RNA, or an RNA component of a RISC complex or RNAi pathway (such as shRNA, miRNA, or siRNA), a regulatory RNA, Piwi-interacting RNAs (piRNAs), small nucleolar RNAs (snoRNAs), a long non-coding RNA (lncRNA) (including intergenic lincRNA, intronic ncRNA, and sense/antisense lncRNA), a long intervening/intergenic noncoding RNA (lincRNA), an enhancer RNA, a bacterial small RNA (sRNA), snRNA, exRNA, scaRNA, Xist, and HOTAIR, and a precursor thereof.

In certain embodiments, the RNA of the disclosure comprises a coding sequence for a protein or polypeptide.

In certain embodiments, protein or polypeptide is a wild-type protein or functional equivalent or variant thereof (such as an enzyme or a structural protein) that can be used to replace a defective protein in a target cell, tissue, or organism.

In certain embodiments, protein or polypeptide is a wild-type protein or functional equivalent or variant thereof (such as an enzyme or a structural protein) that can be used to antagonize the detrimental effect of a compound (small molecule compound, or macromolecules such as lipids, fatty acids, protein, nucleic acid, etc) in a target cell, tissue, or organism.

For example, in certain embodiments, the RNA of the disclosure comprises a coding sequence for an effector enzyme of IscB system (an IscB polypeptide). In certain embodiments, the IscB polypeptide is disclosed in PCT/CN2023/129167 and PCT/CN2023/125069, each of which is incorporated herein by reference in its entirety.

For example, in certain embodiments, the RNA of the disclosure comprises a coding sequence for an effector enzyme of CRISPR/Cas system.

In certain embodiments, the CRISPR-Cas system is a Class 1 system, and the effector enzyme is a type I, III, or IV effector enzyme.

In certain embodiments, the CRISPR-Cas system is a Class 2 system, and the effector enzyme is a type II, V, or VI effector enzyme.

For example, in some embodiments, the effector enzyme is a Class 2, type II enzyme such as Cas9, including Streptococcus pyogenes (SpCas9) or SaCas9 (see WO 2014/093622 (PCT/US2013/074667), incorporated by reference).

In certain embodiments, the Cas effector enzyme is a Class 2, type V Cas protein (Cas12 protein), including Cas12a (formerly known as Cpf1, such as Francisella novicida Cas12a), C2ct, and C2c3, Cas12b, Cas12c, Cas12d, Cas12e, Cas12f, Cas12g, Cas12h, Cas12i, Cas12j, Cas12k, Cas121, Cas12m, Cas12n. Exemplary Cas12 proteins are disclosed in PCT/CN2023/090695 and PCT/CN2023/090685, each of which is incorporated herein by reference in its entirety.

In certain embodiments, the Cas effector enzyme is a Class 2, type VI Cas protein (Cas13 protein), including Cas13a (also known as C2c2), Cas13b, Cas13c, Cas13d, Cas13e, and Cas13f. These Cas proteins use their crRNA to recognize target RNA sequences, rather than target DNA sequences in Cas9 and Cas12a.

Exemplary Cas13 proteins are disclosed in PCT/CN2020/077211, PCT/CN2021/121926, PCT/CN2023/084489, and PCT/CN2022/101884, each of which is incorporated herein by reference in its entirety.

In certain embodiments, the Cas effector enzyme is any one of the Cas effector enzymes described in WO2020/028555 (entire content incorporated herein by reference), including any of Cas9, Cas12 (e.g., Cas12a, Cas12b, Cas12c, Cas12d, etc.), Cas13 (e.g., Cas13a, Cas13b (such as Cas13b-t1, Cas13b-t2, Cas13b-t3), Cas13c, Cas13d, etc.), Cas14, CasX, and CasY.

In certain embodiments, the Cas effector enzyme is fused to a DNA and/or RNA base editor, such as Cytosine or Adenine base editors (CBEs or ABEs). In certain embodiments, the base editor preferantially edits DNA bases and optionally have reduced or substantially no off-target RNA base editing capability. In certain embodiments, the base editor preferentially edits RNA bases and optionally have reduced or substantially no off-target DNA base editing capability. In certain embodiments, the base editor edits both DNA and RNA bases.

In certain embodiments, the base editor is a first, second (BE2), third (BE3), or fourth generation (BE4) base editor.

In certain embodiments, the base editor is a dual base editor.

In certain embodiments, the base editor is an RNA adenosine deaminase (ADAR), such as ADAR1, ADAR2, or ADARDD including ADAR2DD (E488Q).

In any of the above embodiments, the RNA of the disclosure can further comprise a guide RNA sequence designed to be loaded into the encoded CRISPR/Cas effector enzyme for binding to a target polynucleotide sequence complementary to the guide RNA. Such gRNA sequence can be processed by cellular nucleases and be released/separated from the RNA of the disclosure after the RNA of the disclosure has been delivered by the rRAAV viral particles of the disclosure to a target host cell. For example, the gRNA can be present in an unpaired 5′ or 3′ flanking region sequence of a pri-miRNA hairpin structure that is part of the RNA of the disclosure, and, upon processing of the pri-miRNA by cellular enzymes such as Drosha, is released/separated from the primary pri-miRNA transcript.

In certain embodiments, the RNA of the disclosure comprises a coding sequence for an effector enzyme of CRISPR/Cas system, and further comprising a coding sequence for the DNA or RNA base-editing enzyme or domain, such that a fusion of a Cas effector enzyme and the DNA/RNA base-editing enzyme/domain is encoded by the RNA sequence. In certain embodiments, the Cas effector enzyme is defective in nuclease activity, such that it is able to bind to a target polynucleotide sequence through the guide RNA it binds, but is unable to cleave the DNA/RNA target polynucleotide.

In certain embodiments, the RNA of the disclosure comprises a coding sequence for a variant or derivative of the effector enzyme of CRISPR/Cas system, wherein the variant comprises deletions (such as N and/or C terminal deletions, e.g., N-terminal deletion of no more than 210 residues, and/or a C-terminal deletion of no more than 180 residues for Cas13e or Cas13f), insertions, or substitutions of a wild-type CRISPR/Cas system effector enzyme but substantially retains the ability of the wild-type effector enzyme to bind to the gRNA, and/or to cleave the target polynucleotide. In certain embodiments, the variant lacks activity to cleave a target polynucleotide.

In certain embodiments, the RNA base-editing domain encoded by the RNA of the disclosure is an adenosine deaminase, such as a double-stranded RNA-specific adenosine deaminase (e.g., ADAR1 or ADAR2); apolipoprotein B mRNA editing enzyme; catalytic polypeptide-like (APOBEC); or activation-induced cytidine deaminase (AID).

In certain embodiments, the RNA base-editing domain encoded by the RNA of the disclosure comprises an adenosine deaminase and/or a cytidine deaminase, such as a cytidine deaminase acting on RNA (CDAR), such as a double-stranded RNA-specific adenosine deaminase (ADAR) (e.g., ADAR1 or ADAR2), apolipoprotein B mRNA editing enzyme, catalytic polypeptide-like (APOBEC, such as APOBEC1, APOBEC2, APOBEC3A, APOBEC3B, APOBEC3C, APOBEC3D, APOBEC3E, APOBEC3F, APOBEC3G, APOBEC3H, and APOBEC4), activation-induced cytidine deaminase (AID), a cytidine deaminase 1 (CDA1), or a mutant thereof.

In certain embodiments, the ADAR has E488Q/T375G double mutation or is ADAR2DD.

In certain embodiments, the base-editing domain is further fused to an RNA-binding domain, such as MS2.

In certain embodiments, the variant or derivative of the encoded CRISPR/Cas effector enzyme further comprises an RNA methyltransferase, a RNA demethylase, an RNA splicing modifier, a localization factor, or a translation modification factor.

In certain embodiments, the Cas effector enzyme, the variant/derivative, or a functional fragment thereof comprises a nuclear localization signal (NLS) sequence or a nuclear export signal (NES).

In certain embodiments, the Cas effector enzyme, the variant/derivative thereof, or the functional fragment thereof, is fused to a heterologous functional domain. In certain embodiments, the heterologous functional domain comprises: a nuclear localization signal (NLS), a reporter protein or a detection label (e.g., GST, HRP, CAT, GFP, HcRed, DsRed, CFP, YFP, BFP), a localization signal, a protein targeting moiety, a DNA binding domain (e.g., MBP, Lex A DBD, Gal4 DBD), an epitope tag (e.g., His, mye, V5, FLAG, HA, VSV-G, Trx, etc), a transcription activation domain (e.g., VP64 or VPR), a transcription inhibition domain (e.g., KRAB moiety or SID moiety), a nuclease (e.g., FokI), a deamination domain (e.g., ADAR1, ADAR2, APOBEC, AID, or TAD), a methylase, a demethylase, a transcription release factor, an HDAC, a polypeptide having ssRNA cleavage activity, a polypeptide having dsRNA cleavage activity, a polypeptide having ssDNA cleavage activity, a polypeptide having dsDNA cleavage activity, a DNA or RNA ligase, or any combination thereof. In certain embodiments, the heterologous functional domain is fused N-terminally, C-terminally, or internally in the fusion protein.

In certain embodiments, the heterologous functional domain is fused N-terminally, C-terminally, or internally in the fusion protein.

In certain embodiments, the RNA of the disclosure encodes a codon-optimized polynucleotide encoding a wild-type CasPR (e.g., Cas5d, Cas6, or Csf5), a homolog thereof, an ortholog thereof, a paralog thereof, a variant or derivative thereof, or a functional fragment thereof, wherein the polynucleotide is codon-optimized for mammalian (e.g., human) expression, optionally, the wild-type CasPR has the amino acid sequence of anyone of Sequences 1-11. In certain embodiments, the codon-optimized polynucleotide has the amino acid sequence of any one of Sequences 34-44. In certain embodiments, the codon-optimized polynucleotide further comprises sequence encoding a heterologous functional domain. In certain embodiments, the heterologous functional domain comprises an RNA base editor.

In certain embodiments, the RNA of the disclosure encodes a non-naturally occurring polynucleotide comprising a derivative of any one of Sequences 12-33, wherein the derivative (i) has one or more (e.g., 1, 2, 3, 4, 5, 6, 7, 8, 9 or 10) nucleotides additions, deletions, substitutions, and/or other mutations compared to any one of Sequences 12-33; (ii) has at least 20%, 30%, 40%, 50%, 60%, 70%, 80%, 90%, 95%, or 97% sequence identity to any one of Sequences 12-33; (iii) hybridize under stringent conditions with any one of Sequences 12-33, or any of (i) and (ii); or (iv) is a complement of any of (i)-(iii), provided that the derivative is not any one of Sequences 12-33, and that the derivative encodes an RNA (or is an RNA) that has maintained substantially the same secondary structure (e.g., stems, loops, bulges, single-stranded regions) as any of the RNA encoded by Sequences 12-33. In certain embodiments, the derivative functions as a DR sequence for any one of the CasPR, the ortholog thereof, the paralog thereof, the variant thereof, the derivative thereof, or the functional fragment thereof, of the disclosure.

In certain embodiments, the RNA of the disclosure comprises a coding sequence for an engineered Clustered Regularly Interspaced Short Palindromic Repeat (CRISPR)-Cas13 effector enzyme, wherein the engineered Cas13: (1) comprises a mutation in a region spatially close to an endonuclease catalytic domain of the corresponding wild-type Cas13 effector enzyme; (2) substantially preserves guide sequence-specific endonuclease cleavage activity of the wild-type Cas13 towards a target RNA complementary to the guide sequence; and, (3) substantially lacks guide sequence-independent collateral endonuclease cleavage activity of the wild-type Cas13 towards a non-target RNA that does not bind to the guide sequence.

In certain embodiments, the Cas13 is a Cas13a, a Cas13b, a Cas13c, a Cas13d (including CasRx), a Cas13e, or a Cas13f.

In certain embodiments, the Cas13e has the amino acid sequence of SEQ ID NO: 4 of PCT/CN2020/119559 (incorporated herein by reference).

In certain embodiments, the engineered Cas13 of the disclosure has the amino acid sequence of any one of SEQ ID NOs: 6-10 of PCT/CN2020/119559 (incorporated by reference). In certain embodiments, the engineered Cas13 of the disclosure has the amino acid sequence of SEQ ID NO: 9 or 10 of PCT/CN2020/119559

### INCORPORATED BY REFERENCE

In certain embodiments, the engineered Cas13 of the disclosure further comprises a nuclear localization signal (NLS) sequence or a nuclear export signal (NES). In certain embodiments, the engineered Cas13 comprises an N- and/or a C-terminal NLS.

In certain embodiments, the RNA of the disclosure encoding the engineered CRISPR/Cas13 effector enzyme of the disclosure is codon-optimized for expression in a eukaryote, a mammal, such as a human or a non-human mammal, a plant, an insect, a bird, a reptile, a rodent (e.g., mouse, rat), a fish, a worm/nematode, or a yeast.

In certain embodiments, the RNA of the disclosure comprises a coding sequence for the engineered Clustered Regularly Interspaced Short Palindromic Repeat (CRISPR)-Cas13 effector enzyme, the coding sequence having (i) one or more (e.g., 1, 2, 3, 4, 5, 6, 7, 8, 9 or 10) nucleotides additions, deletions, substitutions, and/or other mutations compared to the wild-type sequence; (ii) at least 50%, 60%, 70%, 80%, 90%, 95%, or 97% sequence identity to the wild-type sequence; (iii) hybridize under stringent conditions with the wild-type sequence, or any of (i) and (ii); or (iv) is a complement of any of (i)-(iii).

In certain embodiments, the RNA of the disclosure comprises a coding sequence for a non-coding RNA (ncRNA), such as siRNA, piRNA, short hairpin RNA or shRNA, microRNA or miRNA or precursors thereof including pre-miRNA and pri-miRNA, antisense sequence or oligonucleotide (ASO), guide RNA or gRNA for CRISPR/Cas, rRNA, tRNA, snoRNA, snRNA, exRNA, scaRNA, lncRNA, Xist, and HOTAIR, etc.

### 9. Method of Use

The rRAAV viral particles and RNA sequences of the disclosure can be used to deliver any GOI/RSI to any suitable target cell, tissue, or organism for any use for gene therapy.

In certain embodiments, the rRAAV viral particles and RNA sequences of the disclosure can be used in a method of treatment, in which a defective or loss of function disease gene can be replaced by a functional version of the gene to restore the lost function. For example, in certain embodiments, a wild-type coding sequence, or a variant coding sequence encoding a variant protein of the wild-type protein and having preserved at least one desired functions of the wild-type protein can be delivered to the target cell/tissue/organ, to express the encoded wild-type of variant thereof, in order to compensate for the loss of function of the disease gene.

In certain other embodiments, the rRAAV viral particles and RNA sequences of the disclosure can be used in a method of treatment, in which a defective or gain of function disease gene can be knocked out, knocked down, or otherwise down-regulated by a gene targeting agent to alleviate the detrimental effect of the disease gene. The gene targeting agent can be a CRISPR/Cas effector enzyme (such as an engineered Cas9 or Cas13 effector enzyme as described herein), optionally with a guide RNA that is provided simultaneously (or separately), that together target the disease gene. In certain embodiments, the gene targeting agent can be a Cas effector enzyme linked to a DNA or RNA base editor for DNA-RNA base editing. In certain embodiments, the gene targeting agent is an siRNA, shRNA, microRNA, or antisense RNA.

In certain embodiments, the invention provides a method of modifying a target RNA in a target cell, the method comprising contacting the target cell with an rRAAV viral particle or RNA sequence of the disclosure encoding a CasPR or engineered CRISPR/Cas effector enzyme described herein (or ortholog, paralog, variant, derivative, or functional fragment thereof), wherein a guide sequence for the CasPR/Cas effector enzyme is complementary to at least 15 nucleotides of the target RNA, and wherein the CasPR/engineered Cas effector enzyme associates with the guide sequence to form a complex that binds to and modified the target RNA.

In certain embodiments, the invention provides a method of treating a condition or disease in a subject in need thereof, the method comprising administering to the subject a composition comprising the an rRAAV viral particle or RNA sequence of the disclosure encoding a CasPR or engineered CRISPR/Cas effector enzyme described herein (or ortholog, paralog, variant, derivative, or functional fragment thereof), wherein a guide sequence for the CasPR/Cas effector enzyme is complementary to at least 15 nucleotides of the target RNA, and wherein the CasPR/engineered Cas effector enzyme associates with the guide sequence to form a complex that binds to and modified the target RNA, thereby treating the condition or disease in the subject.

In certain embodiments, the target RNA is modified by cleavage by the CasPR or engineered Cas effector enzyme complex. In certain embodiments, the target RNA is modified by deamination by a derivative comprising a double-stranded RNA-specific adenosine and/or cytidine deaminase. In certain embodiments, the target RNA is an mRNA, a tRNA, an rRNA, a non-coding RNA, an lncRNA, or a nuclear RNA. In certain embodiments, the target RNA is within a cell. In certain embodiments, the cell is a cancer cell. In certain embodiments, the cell is infected with an infectious agent. In certain embodiments, the infectious agent is a virus, a prion, a protozoan, a fungus, or a parasite. In certain embodiments, the cell is a neuronal cell (e.g., astrocyte, glial cell (e.g., Muller glia cell, oligodendrocyte, ependymal cell, Schwan cell, NG2 cell, or satellite cell)).

In certain embodiments, the condition or disease is a cancer or an infectious disease. In certain embodiments, the cancer is Wilms' tumor, Ewing sarcoma, a neuroendocrine tumor, a glioblastoma, a neuroblastoma, a melanoma, skin cancer, breast cancer, colon cancer, rectal cancer, prostate cancer, liver cancer, renal cancer, pancreatic cancer, lung cancer, biliary cancer, cervical cancer, endometrial cancer, esophageal cancer, gastric cancer, head and neck cancer, medullary thyroid carcinoma, ovarian cancer, glioma, lymphoma, leukemia, myeloma, acute lymphoblastic leukemia, acute myelogenous leukemia, chronic lymphocytic leukemia, chronic myelogenous leukemia, Hodgkin's lymphoma, non-Hodgkin's lymphoma, or urinary bladder cancer. In certain embodiments, the method is an in vitro method, an in vivo method, or an ex vivo method. In certain embodiments, upon binding of the complex to the target RNA, the engineered Cas13 does not exhibit substantial (or detectable) collateral RNase activity.

In certain embodiments, the condition or disease is a neurological condition such as glaucoma, age-related RGC loss, optic nerve injury, retinal ischemia, Leber's hereditary optic neuropathy, a neurological condition associated with degeneration of RGC neurons, a neurological condition associated with degeneration of functional neurons in the striatum of a subject in need thereof, Parkinson's disease, Alzheimer's disease, Huntington's disease, Schizophrenia, depression, drug addiction, movement disorder such as chorea, choreoathetosis, and dyskinesias, bipolar disorder, Autism spectrum disorder (ASD), or dysfunction.

In certain embodiments, the method of the disclosure causes one or more of: (i) in vitro or in vivo induction of cellular senescence; (ii) in vitro or in vivo cell cycle arrest; (iii) in vitro or in vivo cell growth inhibition and/or cell growth inhibition; (iv) in vitro or in vitro induction of anergy; (v) in vitro or in vitro induction of apoptosis; and (vi) in vitro or in vitro induction of necrosis.

### EXAMPLES

The following examples are provided to further illustrate some embodiments of the disclosure but are not intended to limit the scope of the disclosure; it will be understood by their exemplary nature that other procedures, methodologies, or techniques known to those skilled in the art may alternatively be used.

### Materials and Methods

### Cell Culture

Human embryonic kidney cells (HEK-293T), mouse embryonic fibroblasts (MEFs) and Hela cells were maintained at 37° C. with 5% CO2 in DMEM (Hyclone, H30243.01) supplemented with 10% fetal bovine serum (Gibco, 10099-141C), 1% MEM Non-Essential Amino Acids Solution (Gibco, 11140050) and 1% Penicillin-Streptomycin-Glutamine (Gibco, 10378016).

### Plasmids

A list of relevant plasmids can be found in Table S5. Plasmids were cloned using PCR amplification with Phanta Max Super-Fidelity DNA Polymerase (Vazyme, P505-d1) and assembled with NEBuilder HiFi DNA Assembly Master Mix (NEB, E262IL).

### Mice

Homozygous Ai9 mice were obtained from the Jackson Laboratory. Heterozygous Ai9 mice were derived from crossing wild-type C57BL/6J females with homozygous Ai9 males. All housing and procedures were performed according to protocols approved by the Institutional Animal Care and Use Committees (IACUC) of HUIDAGENE Therapeutics Co., Ltd. All mice were housed in a room maintained on a 12 h light and dark cycle with ad libitum access to standard rodent diet and water. Animals were randomly assigned to various experimental groups. The AAVs and RAAVs were injected into the hippocampus by stereotaxic injection, and into the mice by intravenous injection.

### Production of AAVs and RAAVs

Both AAVs and RAAVs were produced and purified in an identical manner. HEK293T cells were maintained in DMEM with 10% fetal bovine serum in 150-mm dishes and passaged every 2-3 days. Cells were seeded at 1.5E7 cells per 15 cm dish one day before polyethyleneimine (Polysciences 24765-1) transfection. Then, 15 μg AAV/RAAV transgene plasmid, 15 g AAV/RAAV packaging plasmid and 30 μg pAd-Helper were transfected per plate. The day after transfection, the media was exchanged for fresh DMEM with 2% fetal bovine serum. The supernatants of transfected cells were collected on day 2 and day 5 post transfection. Cells were also scraped with a rubber cell scraper on day 5, pelleted by centrifugation for 10 min at 3000 g, resuspended in 500 μL hypertonic lysis buffer per plate (10 mM Tris base, 150 mM NaCl and 10 mM MgCl2) and lysed via three repeated cycles of freeze/thaw. Add 125 U mL⁻¹ Benzonase nuclease (Sigma, E1014-25KU) to the cell lysate and incubate at 37° C. for 1 h to remove cellular nucleic acids and residual plasmids. The collected supernatants were mixed with a 5× solution of 40% poly (ethylene glycol) (PEG) in 2.5 M NaCl (final concentration: 8% PEG/500 mM NaCl), incubated on ice overnight to facilitate PEG precipitation, and spun at 3000 g for 15 min. The pellet was resuspended in 500 μL lysis buffer per plate and also treated with 100 U mL⁻¹ Benzonase nuclease (Sigma, E1014-25KU) at 37° C. for 1 h. Combine the resuspended virus from concentrated supernatants with cell lysates, and the obtained crude virus were clarified by centrifugation at 3000 g for 10 min and added to Beckman Quick-Seal tubes (Beckman, 342414) via Cotton-plugged Sterile Pasteur Pipets (Kimble, 63B95P). A discontinuous iodixanol gradient was formed by sequentially floating layers: 9 mL 15% iodixanol in lysis buffer with 1 M NaCl, 7 mL each of 25 and 40% iodixanol in lysis buffer, and 5 mL 58% iodixanol in lysis buffer. Phenol red at a final concentration of 1 g mL-1 was added to the 25 and 58% layers to facilitate identification. Ultracentrifugation was performed using a Type 70 Ti rotor in an OPTIMA XE-90 Ultracentrifuge (Beckman Coulter) at 68,000 rpm for 1 h 30 min at 18° C. Following ultracentrifugation, 5 mL of solution was withdrawn from the 40-58% iodixanol interface via a 14-gauge needle, dialyzed with PBS containing 0.001% F-68 using 100-kD MWCO columns (EMD Millipore). The concentrated viral solution was sterile-filtered using a 0.22-μm filter. The final AAV/RAAV preparation was aliquoted and stored at −80° C. until use.

### Extraction and Quantification of Viral Genome

The purified AAVs and RAAVs were first subjected to nuclease treatment (including DNase I and RNase I) at 37° C. for 3 hours to remove unencapsidated DNA and RNA. After nucleases digestion, AAVs and RAAVs were treated with proteinase K (0.5 mg/mL) in a buffer containing 25 mM Tris-HCl (pH7.4), 10 mM EDTA, 100 mM NaCl and 0.5% SDS at 65° C. for about 3 hours to rupture the viral particles and release the packaged genomes. The nuclease-resistant viral genomes were then purified by phenol/chloroform extraction, recovered by isopropanol precipitation (Add 1 μg carrier DNA to each sample), and dissolved in nuclease-free Water.

The extracted viral genomes were directly subjected to qPCR to quantify the viral DNA titer. To quantify the viral RNA titer, extracted genomes were first digested with gDNA wiper Mix (Vazyme, R223-01) to remove viral DNAs. Undigested viral RNAs were then reverse-transcribed into cDNAs and quantified via qPCR. Pairs of primers (Table S6) were designed targeting AAV/RAAV genomes.

### Transmission Electron Microscopy Analysis

In sample preparation for negative stain-electron microscopy, 10 μL purified AAVs and RAAVs were dropped on a 300 meshes copper grid coated with a continuous carbon film. The sample was allowed to adsorb for 2 minutes after which excess solution was removed with kimwipes. Subsequently, 10 μL negative staining solution containing 3% aqueous phosphotungstic acid was dropped on the TEM grid and incubated for 2 minutes, the excess solution was removed by touching the edge with kimwipes. The sample was allowed to dry before observation under a Talos L120C transmission electron microscope with a magnification of 73,000. Images were taken using Thermo Scientific™ CETA 16 4K×4K CMOS camera.

### Silver Stain

Samples from purified AAV/RAAV vectors were loaded onto 4-20% Bis-Tris Gradient Precast Gels (Tanon, 180-9115H) and ran using 1×MOPS running buffer (Tanon, BT8100-2002). The gels were stained with Fast Silver Stain Kit (Beyotime, P0017S).

### Sedimentation Velocity AUC

Sedimentation velocity analytical ultracentrifugation (SV-AUC) analysis was performed using a Proteome Lab XL-I (Beckman Coulter, Indianapolis, IN). 400 μL of the sample was loaded into the sample sector of a two-sector velocity cell, and 400 μL of PBS (containing 0.001% F-68) was loaded into the corresponding reference sector. The sample was placed in the four-hole rotor and allowed to equilibrate in the instrument until a temperature of 20° C. and a full vacuum were maintained for one hour. Sedimentation velocity centrifugation was performed at 20,000 rpm and 20° C. Absorbance (260 nm) optics were used to record the radial concentration as a function of time until the lightest sedimenting component cleared the optical window (1.2 hours).

The percentage of virions containing a full genome was determined by analyzing approximately 200 scans using the absorbance detection method and the SEDFIT (NIH/ www.analyticalultracentrifugation.com) continuous-size C(S) distribution model. Second (2nd) derivative regularization was applied to the fitting with a confidence level of F statistic=0.68. The following C(S) parameters were held constant: resolution=200S, S min=1, S max=200 and frictional ratio=1.0. RI and TI noise subtractions were applied, and the meniscus position was allowed to float, letting the software choose the optimal position. This model fits the data to the Lamm equation, and the resulting size distribution is a “distribution of sedimentation coefficients” that resembles a chromatogram with the area under each peak proportional to the concentration in units of Fringes or OD260 units. The sedimentation coefficient in Svedberg units and the relative concentration in OD units were determined for each component in the distribution. The results of the AUC analyses are plotted as the normalized differential coefficient distribution value, C(S), vs. sedimentation coefficient (S).

### Analysis of Viral Genome on Agarose Denaturing Gel

Mix 10 ng viral genomes with a 0.5 volume of Glyoxal Load Dye (Invitrogen, AM8551), and incubate the samples at 50° C. for 1 h. The denatured viral genomes were subsequently separated on a 1% glyoxal denaturing agarose gel (added 1/5000 SYBR™ Green II) at room temperature for 1 h. The image was taken by the Tanon 2500 series automatic gel image analysis system.

### Mouse Embryonic Fibroblasts (MEFs) Isolation

Embryos from homozygous Ai9 mice were isolated between E12.5 and E18.5. After the heads, tails, limbs, and most of the internal organs were removed, the embryos were minced and trypsinized for 20 min and then seeded into 10 cm cell culture dishes in 10 mL of complete DMEM media. The cells were split at 1:2-1:3 ratios when freshly confluent, passaged two or three times to obtain a morphologically homogenous culture, and then frozen or expanded for further studies.

### HEK293T Cre Reporter Cell Line Generation

HEK293T Cre reporter cell line was generated with the PiggyBac transposon system. A PB-T-loxP-tdTomato cassette was generated by subcloning the loxP-tdTomato cassette from Ai9 (Addgene #22799) into a PB-T plasmid. HEK293T reporter cell lines were created by seeding cells at 50% confluency in 6 well plates. The following day, the PB-T-loxP-tdTomato construct were cotransfected with the helper plasmid pCAG-PBase using polyethylenimine. Transfected cells were selected in puromycin (Thermo Fisher, A1113803) for 2 weeks and then sorted based on GFP on a BD FACSAria™ III Cell Sorter. Single sorted-cells were deposited into 96-well plates to get monoclonal cell lines.

mRNA-Sequencing of Whole Cell RNA and Viral Vector Genomes

RAAV and its no-MCP control were generated (with ten 15 cm-dishes per group) and VLP RNA was extracted as viral vector genome extraction described above. Whole cell RNA was extracted with TRizol Reagent (Invitrogen, 15596018) and purified using the phenol-chloroform extraction method. Subsequently, 1 μg RNA was used for the following library preparation. To mitigate potential bias resulting from the small amount of VLP RNA used during library preparation, the inventor supplemented the viral vector genomes with 1 μg of carrier RNA. The poly(A) mRNA isolation was performed using Oligo(dT) beads, and the multiplexed RNA sequencing library was prepared using VAHTS® Universal V8 RNA-seq Library Prep Kit for Illumina (Vazyme, NR605). Libraries were sequenced on the Illumina novaseq 6000 using a 2×150 paired end (PE) configuration according to the manufacturer's instructions. Quality control was performed using Cutadapt (V1.9.1, phred cutoff: 20, error rate: 0.1, adapter overlap: lbp, min. length: 75, proportion of N: 0.1). Clean data were aligned to reference genome (GRCh38.p13+optCre) via software Hisat2 (v2.2.1). Differential gene expression analysis was performed using the DESeq2 Bioconductor package, a model based on the negative binomial distribution. Estimation of dispersion and logarithmic fold changes incorporate data-driven prior distributions, with Padj of genes set to <=0.05 to detect differentially expressed ones. Full read alignments were generated using Geneious prime.

### AAV and RAAV Infection

For all Ai9-MEFs infection experiments, cells were plated on 48-well plates at a density of 5E4 cells per well 24 hours before infection. Purified AAVs and RAAVs were added to Ai9-MEFs in triplicate. Vector genome titer was used for MOI calculation. Infected cells were collected at different time points for analyzing Cre DNA, Cre RNA, and Cre protein, or maintained for 5 days before flow cytometry analysis. To investigate the source of viral RNAs in infected cells, the transcription inhibitor—actinomycin D (AAT Bioquest 17505) was added to the cells at a concentration of 5 g/mL 2 hours post-infection.

For HEK293T Cre reporter cell line infection experiments, cells were plated on 48-well plates at a density of 8E4 cells per well 24 hours before infection. Purified AAVs and RAAVs were added to HEK293T Cre reporter cells in triplicate. Vector genome titer was used for MOI calculation. Cells were maintained for 5 days before flow cytometry analysis.

qPCR and RT-qPCR

Total cellular DNA was extracted with TIANamp genomic DNA kit (TIANGEN, DP304-03). Total cellular RNA was extracted with TRIzol Reagent (Invitrogen, 15596018) and purified using the phenol-chloroform extraction method. Total RNA was reverse transcribed using the HiScript II Q RT SuperMix for qPCR (+gDNA wiper) (Vazyme, R223-01) according to the manufacturer's guidelines. Gene-specific primers used for qPCR and RT-qPCR are shown in Table S6. qPCR was performed using AceQ qPCR SYBR Green Master Mix (Vazyme, Q 111-02) on a CFX96 Touch™ Real-time PCR System (Bio-Rad) according to manufacturer's guidelines.

### Flow Cytometry Analysis

Five days after transduction, transduced Ai9-MEFs or HEK293T Cre reporter cells were washed once with 1×PBS and dissociated with 0.25% trypsin-EDTA. Cells were resuspended with DMEM (containing 10% FBS), and the rescued tdTomato signals were determined using flow cytometry (Beckman CytoFlex). Analysis was performed using FlowJo v10.7 (BD Biosciences). Representative gating schemes are shown in Figure S5.

### RNAScope Assay

HeLa (human cervical carcinoma) cells were seeded in 8-well glass chamber slides (MERCK, #PEZGS0816) at a density of 8E3 cells per chamber 24 hours before infection. Cells were then infected with RAAV-DJ (MOI=10,000 vg) or AAV-DJ (MOI=1,000 vg) in DMEM (containing 2% FBS). BafilomycinA1 (Selleck, #S1413) was applied to the cells 1 h prior to infection at a concentration of 100 nM and kept in the medium for 24 h, concomitantly with the infection. At 1 or 6 h after transfection, actinomycin D (AAT Bioquest, #17505) was added at the final concentration of 5 μg/ml. At various time points post-infection, the cells were fixed and processed for RNAscope analysis.

RNAscope assay was performed according to the manufacturer's protocols of RNAscope™ Multiplex Fluorescent Reagent Kit v2 (ACD, #323100). Briefly, fixed cells were pretreated using the Universal Pretreatment Reagents (ACD, #322380). The chemically modified Cre probe (ACD, #474001) consists of 22 ZZ pairs. The pretreated cells were hybridized with the target probes at 40° C. for 2 h and labeled with TSA Vivid fluorescent dye 520 (ACD, #323271) at a concentration of 1:1000. Nuclei are visualized using DAPI staining. The imaging was performed using a confocal microscope (Nikon C2si, Nikon).

### Western Blot

For all the western blotting experiments, cells were lysed in LDS Sample Buffer (Biofuraw 180-8201D). The proteins were separated using SDS-polyacrylamide gel electrophoresis and transferred to polyvinylidene difluoride membranes. The membranes were blocked by 5% fat-free milk dissolved in TBS/0.05% Tween-20 (TBST) for 1 h, and incubated with anti-Cre monoclonal antibodies (1:1000, Cell Signaling Technology, 15036S) at 4° C. for 3 hours, washed 5 times in 1×TBST, incubated with anti-rabbit secondary antibodies (1:1000, Cell Signaling Technology, 7074S) for 1 h at room temperature, washed 3times in 1×TBST, then imaged with Tanon 4600. Tubulin detected using anti-tubulin polyclonal antibodies (1:3000, Bioworld, AP0064).

Stereotaxic Injection (into Hippocampus) & Intravenous Injection

To investigate the infectivity of AAVs and RAAVs in mice hippocampus, Ai9 Mice (8 weeks old) were anesthetized with a mixture of zoletil (60 g/g) and xylazine (10 μg/g), and then unilatrally, stereotactically injected with 1 μL AAV-Cre (two doses were set: 1E8 vg/mouse and 1E7 vg/mouse) or 1 μL RAAV-Cre (1E8 vg/mouse) into the dentate gyrus region of the right hippocampus according to the following coordinates: anteroposterior (A/P)=−1.7 mm, mediolateral (M/L)=−1.0 mm, dorsoventral (D/V)=−2.1 mm.

To investigate the tropisms of AAVs and RAAVs in mice brain, Ai9 Mice (8 weeks old) were anesthetized and intravenously injected with 300 μL AAV-Cre (1E11 vg/mouse) or RAAV-Cre (three doses were set: 1E11 vg/mouse, 3E11 vg/mouse, and 1E12 vg/mouse).

### Immunofluorescence Staining and Imaging of Tissues.

To investigate the infectivity and persistence of AAVs and RAAVs in mice, paraformaldehyde-fixed cryostat tissue section samples (brain and liver) were prepared 4 weeks after injection. Tissue sections were stained with anti-Cre antibodies (1: 800, cell signaling technology, 150365) and followed by Alexa Fluor 488-AffiniPure Donkey Anti-Rabbit IgG (H+L) (1: 1000, Jackson ImmunoResearch, 711-545-152). The nuclei were stained by DAPI (D3571, Invitrogen) and mounted with SlowFade Diamond Antifade Mountant (Invitrogen, S36972) on glass slides. The imaging was performed using a confocal microscope (Nikon C2si, Nikon).

gRNA Guide Cell Line Generation

A guide against human TTR gene was cloned using NEBuilder HiFi DNA Assembly under the control of a U6 promoter into a custom PB-T vector. HEK293T cells were seeded at 50% confluency in 6 well plates. The following day, the PB-T-U6-gRNA construct was cotransfected with the helper plasmid pCAG-PBase using polyethyleneimine. Transfected cells were selected in puromycin (Thermo Fisher, A1113803) for 2 weeks and then sorted based on BFP on a BD FACSAria™ III Cell Sorter. Single-sorted cells were deposited into 96-well plates to get monoclonal cell lines.

### Indel Sequencing of In Vitro Edited Cells

In vitro, 96-well plates of tissue culture cells were infected with AAV-DJ-Cas12Max and RAAV-DJ-Cas12Max, and cells were lysed with 20 μL lysis buffer from One Step Mouse Genotyping Kit (Vazyme, PD101-01) 5 days after infection. The target region was amplified from genomic DNA by nested PCR with primers described in Table S7. Barcoded PCR products were pooled together, purified with Gel Extraction Kit (OMEGA, D2500-02), and sequenced on an Illumina HiSeq system (150-bp paired-end reads). Indels were quantified from the resulting library using the script which has been deposited on github (https://github.com/yszhou2016/Cas12f/blob/main/0.Cas-Finder/3.Indel_Calculate.pl).

### Helicase Sequence Alignment and Phylogenic Analysis

The inventor downloaded 98 viral protein sequences that contain a SF3 helicase (22 ssDNA viral helicases and 76 ssRNA viral helicases) from Genbank and Uniprot (Table. S1). The core sequence of these 98 helicases were aligned and phylogenetically analyzed via AlignX (FIGS. 27A and 27B). These 98 core sequences were also aligned by MUSCLE, and full sequences of randomly selected 23 helicase-containing proteins were aligned via AlignX (FIGS. 27C and 27D). The sequences in the alignment were arranged according to their genetic similarities determined by the phylogenetic tree (FIG. 27).

### Statistics

Data were analyzed using GraphPad Prism 8. Quantitative data are presented as mean SD with n 3 biological replicates per condition. Unless otherwise stated, biological replicates represent independent treatments in separate virus batches, culture wells or mice. Statistical significance was computed using unpaired t-test. The specific statistical method applied, and descriptions of replicates are provided in the figure legends. The asterisks indicate statistical significance; unless otherwise specified, *P<0.05, **P<0.01, ***P<0.001, ****P<0.0001; ns, non-significant.

### Example 1: Multi-Step AAV Engineering for RNA-Carrying Capability

AAV is conventionally produced by co-transfection of transgene plasmid, packaging plasmid (Rep-Cap plasmid), and helper plasmid (pAd-Helper).

In the first step of the new AAV design of the Example, the inventor removed both ITRs from the transgene plasmid (expressing tdTomato) and introduced RNA-packaging signal (RPS) of either one or three copies of MS2 stem-loops (1× or 3×MS2) at the 3′ end of the transgene cassette (between WPRE and poly-A tail). The construct of this transgene plasmid did not comprise 5′ ITR and 3′ ITR and comprised, from 5′ to 3′, promoter (CAG), Kozak sequence, tdTomato coding sequence (transgene), WPRE, 1× or 3×MS2, and SV40 poly(A) signal (FIG. 22B). In parallel, a transgene plasmid with no ITR and no RPS (MS2) was constructed as a negative control (FIG. 22B). See Table B for the sequence of the RNA-packaging signals.

For the packaging plasmid, the inventor fused MS2 coat protein (MCP; capable of binding MS2; serving as an RPS-binding protein (RBP)) to the N-terminus of AAV2 Rep78/68 (the fusion comprised, from N- to C-terminus, MP (the first two amino acids of AAV2 Rep78/68), MCP, and the remaining AAV2 Rep78/68) on the packaging plasmid to enable its binding to MS2 in the specific RNA transcribed from the RPS-carrying transgene plasmid. This packaging plasmid comprised, from 5′ to 3′, a polynucleotide encoding RBP-Rep fusion protein and a polynucleotide encoding AAV-DJ Cap protein (FIG. 22B). Another packaging plasmid with no MCP was used as a negative control (FIG. 22B). See Table A for the sequences of the RBPs. See Table C for the sequences of the RBP-Rep fusion proteins.

The inventor co-transfected HEK293T cells with the new RPS-carrying transgene plasmid and the new RPS-binding protein-carrying packaging plasmid in the presence of the pAd-Helper to produce the RNA-carrying AAVs (termed “RAAVs”) (FIG. 22B). The Inventor also generated conventional AAVs for comparison (FIG. 22A).

After harvesting RAAV particles from the producer cells and supernatants, the inventor analyzed the packaged nucleic acid of the RAAV particles to determine whether the AAV capsid of the RAAV particles packaged the MS2-containing RNA. To avoid the high background plasmid signals, the inventor treated the virus stock with nucleases before extracting AAV capsid-protected (packaged) nucleic acids. The extracted nuclease-resistant RNA and DNA (if any) were quantified by RT-qPCR and qPCR. In addition, two pairs of qPCR primers were designed to distinguish packaged DNAs and packaged RNAs. Specifically, CAG-targeting primers were used for detecting DNA only, while WPRE-targeting primers detected both DNA and RNA. As expected, packaging of DNA was essentially eliminated after removing ITRs from the transgene plasmid, since the detected DNA titer was about 4 orders of magnitude lower than that found in the conventional AAV prepared by using transgene plasmid with ITRs (FIGS. 22C and 22D). In the meantime, RNA-containing RPSs with 1× or 3×MS2 were efficiently packaged into AAV capsids, which is believed to be due to the introduction of MCP-fused Rep78/68 into the packaging system after removing ITRs. This resulted in RNA titers for ‘1×MS2’ and ‘3×MS2’ groups 47- and 127-fold of that found for the no MCP group, respectively. The inventor thus used the 3×MS2 & MCP combination as the first generation RAAV system (termed “RAAV-v1”) (FIGS. 22C and 22D). The inventor also demonstrated that Cre mRNA could be packaged with a titer similar to that of tdTomato mRNA (FIG. 22E), indicating that the RAAV-v1 is effective for packaging various RNA transgenes.

Although most of the packaged nucleic acids in the above RAAVs were found to be RNA, the inventor detected a small number of DNAs (4.8% of the packaged nucleic acids) (FIG. 22E). The inventor reasoned that these DNAs in RAAV vectors might come from non-specific recognition and cleavage of the transfected plasmid by the endonuclease activity of expressed Rep78/68 proteins. The inventor thus constructed “RAAV-v2” system by introducing ‘Y156F’ mutation to the MCP-fused Rep78/68 protein (MCP-Rep78/68^{Y156F}) in RAAV-v1 to abolish the endonuclease activity but retain the helicase/ATPase activity of Rep78/68 protein (FIG. 22F). RAAV-v2 indeed showed significantly reduced DNA packaging without affecting the RNA packaging, as compared to that found for RAAV-v1 (FIG. 22G).

To demonstrate that the approach of using RPS & RBP in AAV engineering to generate RAAVs could be generalized, the inventor examined the RNA-packaging efficiency of RAAV-v2 system using two additional pairs of RPS & RBP: (1) PP7 binding site and PP7 bacteriophage coat protein (PP7/PCP) and (2) Com binding site and phage COM protein (com/COM). Transgene plasmids harboring three copies of RPS (3×PP7 or 3×com) and their corresponding packaging plasmids (containing PCP- or COM-fused Rep78/68^{Y156F}) were constructed. RAAVs were produced, purified, and titrated as described above. The inventor found that both PP7/PCP and com/COM pairs indeed conferred markedly elevated RNA packaging capability of RAAV, compared to that found for the conventional AAV (FIG. 22H). In addition, the inventor tested more AAV capsids (capsid 5, 8, and 9) and found that the RNA packaging efficiency of RAAVs (with MS2/MCP or com/COM pair) was slightly higher when AAV capsids (capsid 5, 8, and 9) other than capsid DJ were used (FIG. 221).

### Example 2: Helicase Engineering and Cargo Sequence Optimization

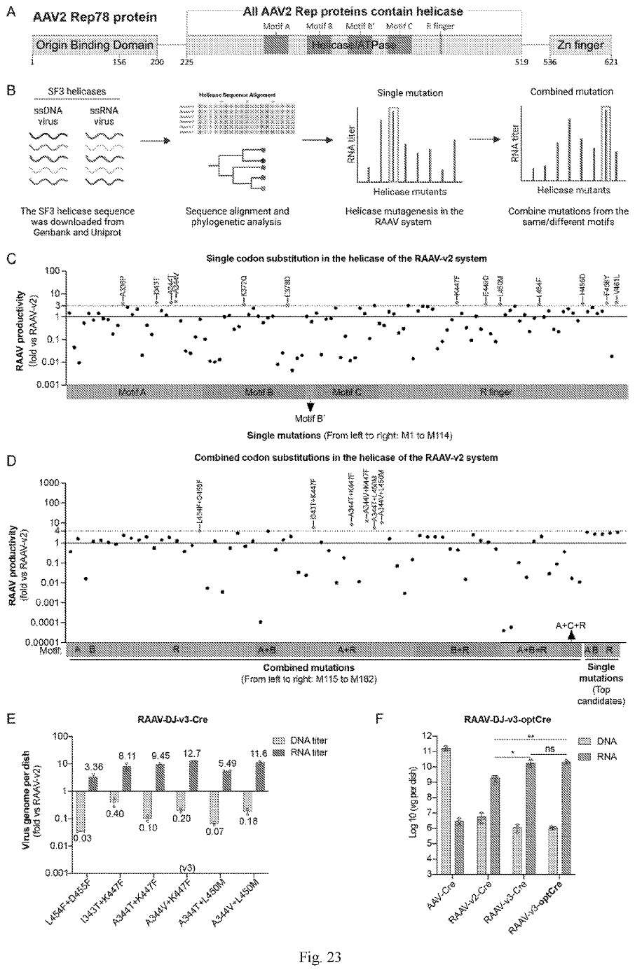

Although the above engineering procedure substantially improved specific RNA packaging of RAAV and reduced undesired DNA packaging, it would be desired to further increase the titer of packaged RNA in RAAV. The Rep proteins of AAV contained a helicase/ATPase (“helicase” for short) for DNA packaging (FIG. 23A). For example, Rep proteins of AAV2, i.e., AAV2 Rep78 (SEQ ID NO: 88), AAV2 Rep68 (SEQ ID NO: 284), AAV2 Rep52 (SEQ ID NO: 285), and AAV2 Rep40 (SEQ ID NO: 286), share a common helicase domain (SEQ ID NO: 186). Although the above results demonstrated that this helicase could also translocate RNA, the inventor surmised that the RNA packaging efficiency may be elevated by engineering helicase via mutagenesis. AAV helicases belong to the superfamily 3 (SF3) helicases, which contain four conserved motifs, Motif A, Motif B, Motif B′, and Motif C that constitute the core of the helicase domain (FIG. 23A). A conserved arginine finger is located after motif C. SF3 helicases could be encoded by both DNA and RNA viruses. The inventor downloaded 98 SF3 helicase-containing viral protein sequences (22 ssDNA and 76 ssRNA viral proteins) from GenBank and UniProt (Table S1). The helicase domains of these 98 helicases were aligned and phylogenetically analyzed via AlignX (FIG. 23B; FIGS. 27A and 27B). They were also aligned by MUSCLE, and the full sequences of randomly selected 23 helicase-containing viral proteins were aligned via AlignX (FIGS. 27C and 27D). These analyses revealed multiple highly conserved regions across all viral helicases, as well as some divergent positions (loci) between ssDNA and ssRNA viruses (FIGS. 27B to 27D). For example, each amino acid residue of the helicase domains of all the 22 ssDNA viruses at a position corresponding to position 344 (numbered according to SEQ ID NO: 88; or position 37 numbered according to SEQ ID NO: 186) of the helicase domain (SEQ ID NO: 186) of AAV2 Rep78 (SEQ ID NO: 88) are A, e.g., A344 (numbered according to SEQ ID NO: 88; or position 37 numbered according to SEQ ID NO: 186) of the helicase domain (SEQ ID NO: 186) of AAV2 Rep78 (SEQ ID NO: 88), A346 (numbered according to SEQ ID NO: 94; or A37 (numbered according to SEQ ID NO: 192)) of the helicase domain (SEQ ID NO: 192) of AAV8 Rep78 (SEQ ID NO: 94), whereas most (54) of the amino acid residue of the helicase domains of the 76 ssDNA viruses at a position corresponding to position 344 (numbered according to SEQ ID NO: 88; or position 37 numbered according to SEQ ID NO: 186) of the helicase domain (SEQ ID NO: 186) of AAV2 Rep78 (SEQ ID NO: 88) are not A. Thus, the position (locus) of the helicase domains of ssDNA viruses corresponding to position 344 of the helicase domain (SEQ ID NO: 186) of AAV2 Rep78 (SEQ ID NO: 88) is a divergent position (locus) between ssDNA and ssRNA viruses.

The inventor focused on the divergent loci within conserved regions and identified 114 amino acids as candidates for mutagenesis (black boxes in FIG. 27B to 27D). The inventor conducted single substitutions in the helicase domain (SEQ ID NO: 186) shared by AAV2 Rep proteins (SEQ ID NOs: 88 and 284-286) of the RAAV-v2 at the 54 positions in Table S2, e.g., G325, K326 . . . F458, V461, and identified 53 of 114 single substitutions (at 30 positions: G325, R327, W331, A336, T337, I343, A344, D371, K372, M373, I375, E378, C405, T419, S420, T422, C425, Q442, D443, M445, K447, E449, L450, T451, L454, D455, H456, D457, F458, V461) that exhibited increased RNA packaging efficiency than negative control without such substitution and 13 of 114 single substitutions that exhibited over 3-fold enhancement in RNA packaging efficiency than negative control without such substitution (FIG. 23C, Table S2).

Subsequent motif-directed combinatorial mutagenesis for multiple (2 to 5) amino acid substitutions, based on the above single substitutions, resulted in 68 mutated helicase domains with multiple mutations at the same or different motifs. 6 of the 68 mutated helicase domains yielded RNA packaging capability at least 4.1-fold of that found for negative control without such substitution. The combination mutations of A344V and K447F achieved the highest (12.7-fold) RNA packaging efficiency (FIGS. 23D and 23E, Table S3), and this mutated helicase domain/mutated AAV2 Rep78/68/52/40 was used as “RAAV-v3” system in the subsequent experiments. Furthermore, the DNA titer of this RAAV-v3 was 5-fold lower than that of RAAV-v2 (FIG. 23E). Overall, the RAAV-v3 system now achieved ~6,000-fold higher RNA packaging and ~14,100-fold lower DNA packaging, as compared with those of conventional AAV. Consequently, the RNA titer in RAAV-v3 was only 8.93-fold lower than the DNA titer in AAV (FIG. 23F). Although the helicase mutations tested herein are based on the helicase domain (SEQ ID NO: 186) of AAV2 Rep proteins (SEQ ID NOs: 88 and 284-286), similar or corresponding mutations of Rep proteins from other AAV or DNA viruses, e.g., AAV1, AAV2, AAV3, AAV3A, AAV3B, AAV4, AAV5, AAV6, AAV7, AAVrh74, AAV8, AAV9, AAV10, AAV11, AAV12, AAV13, AAVrh10, are also within the scope of the disclosure.

In addition to the capability of selective RNA packaging, the efficiency of expressing exogenous proteins in cells by RAAVs may also depend on the translation of the delivered RNA. Thus, the inventor further pursued codon optimization of RNA to improve its protein translation. The Cre-coding sequence was optimized using two codon optimization online tools (Table S4). The inventor introduced RAAV (with capsid DJ) carrying optimized Cre-coding sequences (RAAV-Cre opt1 to opt4) into cultured mouse embryonic fibroblasts (MEFs) isolated from Cre reporter mice (Ai9 mice harboring loxP-tdTomato). The RAAV-v3 carrying original Cre-coding sequences (RAAV-Cre) was used for comparison. Five days after infection, the inventor found that RAAV-Cre opt4 yielded significantly higher percentages of tdTomato* cells than RAAV-Cre at the same multiplicity of infection (MOI, calculated as vector genomes per cell) without affecting the RNA titer (FIG. 28), indicating elevated Cre protein expression. Therefore, RAAV-v3 with Cre mRNA using opt4 (RAAV-v3-optCre), generated by the combination of helicase mutagenesis and Cre coding sequence optimization, was currently the most efficient RAAV vector (FIG. 23F) and was used in all following experiments, except indicated otherwise.

### Example 3: Further Characterization of RAAVs

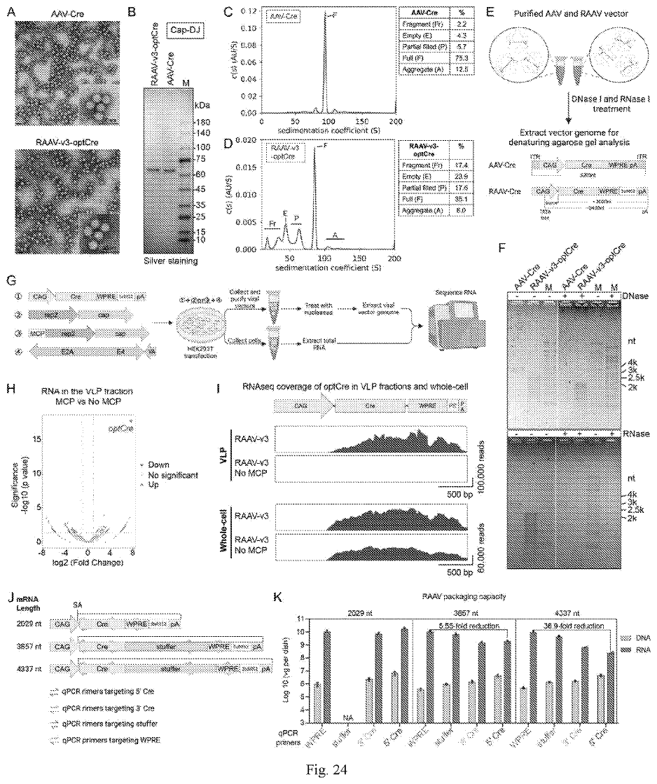

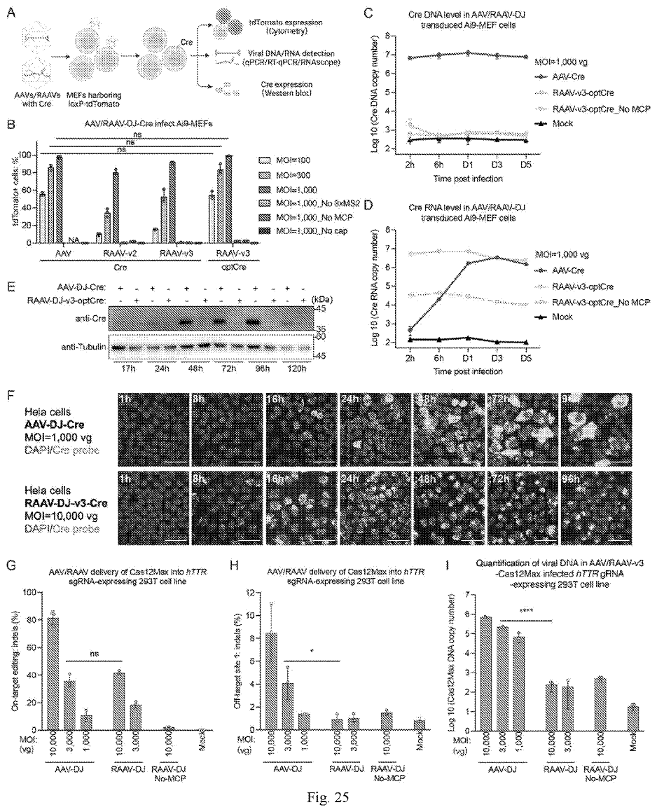

A comprehensive assessment of the properties of RAAV-v3-optCre was further conducted, using silver staining of protein composition of the RAAV with the SDS-PAGE method and visualization of RAAV particles with transmission electron microscopy. The inventor found that RAAVs and AAVs were indistinguishable in the capsid composition and morphology (FIGS. 24A and 24B). The particle heterogeneity of RAAV-v3-optCre was further examined with sedimentation velocity analytical ultracentrifugation (SV-AUC). The RAAV-v3-optCre vector containing the full-length RNA was represented by the sedimentation peak at 88S, which accounted for 35.1% of the entire RAAV-v3-optCre preparation (FIG. 24D) and lower than that found for the conventional AAV preparation (75.3%) (FIG. 24C). Further optimization of production and purification processes may improve the relative amount of intact RNA-loaded RAAV, as that commonly practiced for improving AAV quality for translational applications (16, 29).

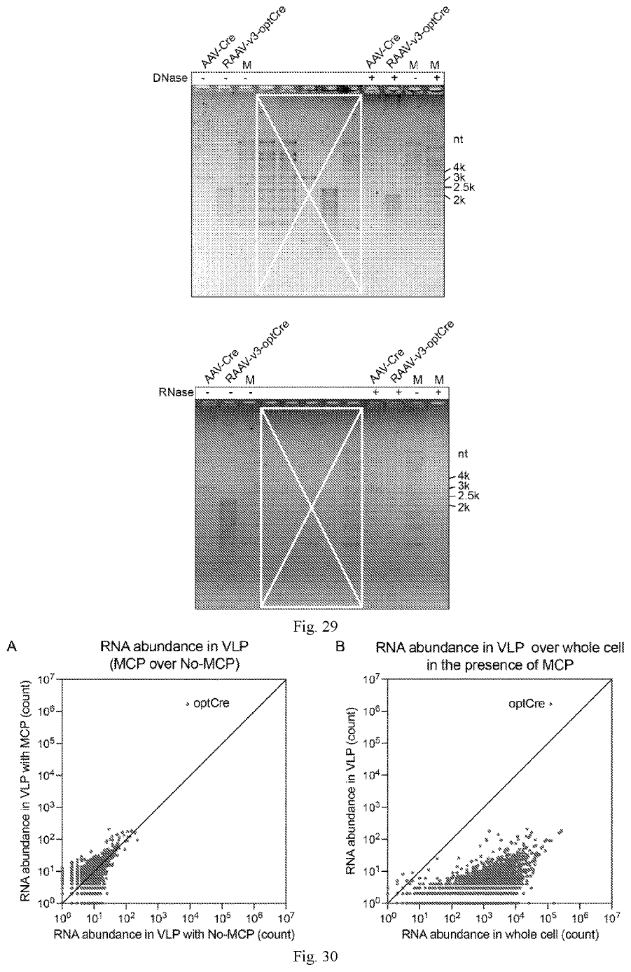

To assess the specificity and integrity of the genome of RAAV, the inventor extracted and analyzed the genomes of RAAV-v3-optCre and AAV-Cre on denaturing agarose gels stained with SYBR™ Green II. The inventor observed a 2000-2400 nt band (consistent with the expected size of Cre mRNA in RAAV) that was resistant to DNase I but not RNase I, indicating that most packaged genomes in RAAV were intact RNAs (FIGS. 24E and 24F, FIG. 29), whereas the 3265 nt-long ssDNA in AAV-Cre was highly susceptible to degradation by DNase I but not RNase I. In addition, RNA sequencing on whole-cell lysate and virus-like particle (VLP) fraction (after removing residual unencapsidated RNA by nuclease) was performed to identify various RNA species in the VLP fraction (FIG. 24G), with and without the presence of MCP. The inventor found that MCP markedly and specifically elevated the amount of full-length optCre transcripts in the VLP fraction, but only slightly increased the optCre transcripts in whole-cell lysates (FIGS. 24H and 24I, FIG. 30).

Conventional AAV has an upper limit for packaging ssDNA of ~4.7K nt. The inventor next examined the packaging capacity of RAAV-v3 by generating RAAV that contained RNAs of different lengths (2029, 3857, and 4337 nt), with identical sequences at both ends. These RAAVs were then titrated with 4 pairs of qPCR primers that targeted different regions of the RNA genome (FIG. 24J). The inventor found no difference in the titers of WPRE and the stuffer (with primers more proximal to RPS) among various RAAVs with different genome sizes (FIG. 24K). However, the titers of 3′-Cre and 5′-Cre (with primers more distal to RPS) decreased with increasing RNA lengths. Compared with the WPRE titer, 5′-Cre titer was 5.6-fold and 36.9-fold lower for 3857 and 4337 nt RNAs, respectively (FIG. 24K). Thus, the size of the RNA may impact its full-length packaging in RAAV, and this may also be affected by other factors, like RNA stability and heterogeneity.

### Example 4: Transient Protein Expression of RAAV-Delivered RNA in Cells

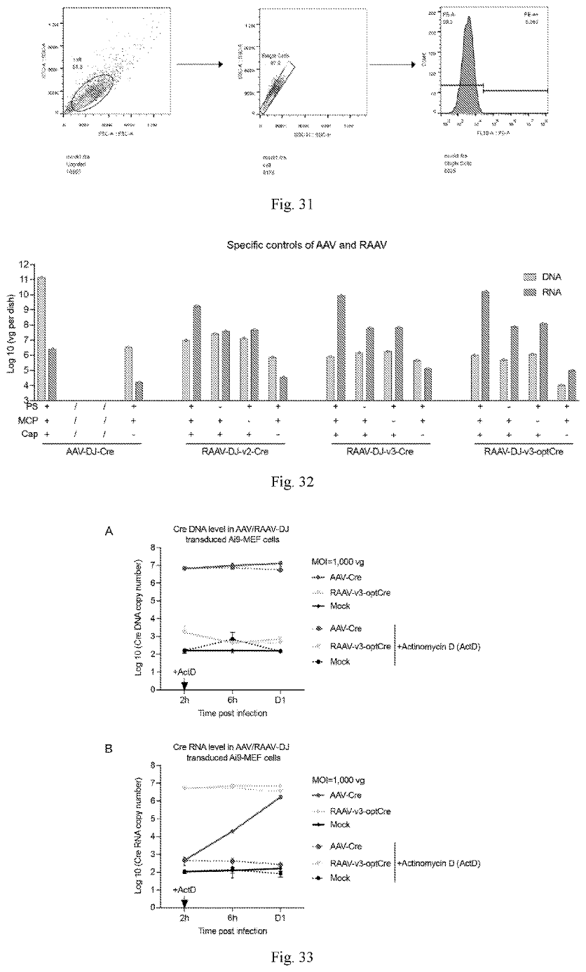

Next, the inventor explored the cell infectivity of RAAVs in parallel with conventional AAVs by the Cre reporter system (loxP-tdTomato), and the vector genome (vg) titer was used for the MOI calculation. RAAV-Cre and conventional AAV-Cre vectors were applied for 12 hours to cultured Ai9-MEFs, and the percentage of tdTomato* cells was analyzed by flow cytometry 5 days after infection (FIG. 25A). The results showed that RAAV-v2-Cre, RAAV-v3-Cre, and RAAV-v3-optCre all achieved the successful transfer of Cre mRNA and expression of functional Cre protein, as indicated by tdTomato fluorescence, and the infection ability of RAAV-v3-optCre was comparable to that of AAV-Cre (FIG. 25B, FIG. 31). By contrast, negative controls of RAAV-v3-optCre (no MS2, no MCP, or no Cap) were also prepared and none showed obvious infectivity (FIG. 25B). For the infection assay, the DNA amount was normalized among RAAV, RAAV with no MS2, and RAAV with no MCP, and the infection volume was normalized between RAAV and RAAV with no Cap. Since RAAV negative controls contained significantly lower amounts of RNA than RAAV (FIG. 32), the infectivity of RAAV could be attributed to encapsidated RNA, rather than residual DNA.

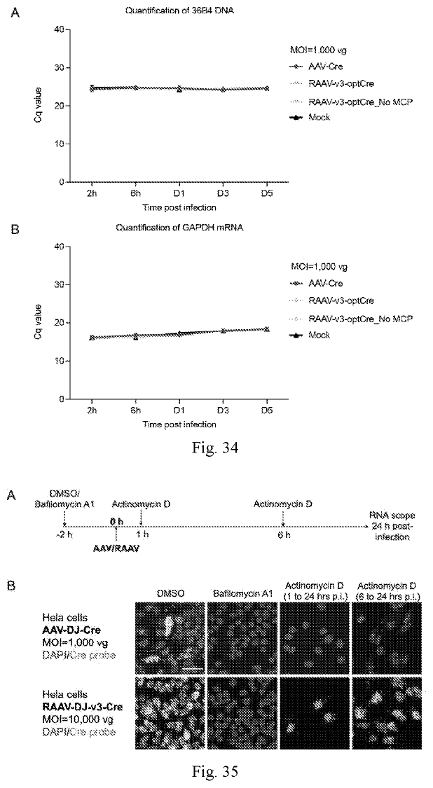

To determine the exact level and lifespan of the viral vector-derived mRNA and the translated Cre recombinase, the inventor infected cultured Ai9-MEFs with RAAV-v3-optCre, and the cells were collected at various time points for assaying the amounts of Cre DNA, mRNA and protein (FIG. 25A). The inventor found that the Cre DNA level in cells infected with RAAV with and without MCP was the same as the background level found in uninfected control (MOCK) cells (FIG. 25C). The RAAV-derived mRNA could be detected in these cells as early as 2 hours post-infection, peaked at 6 hours, and decreased afterward to a lower level of ~30% of the peak level by Day 5 (FIG. 25D). By contrast, the amount of Cre mRNA in AAV-Cre-infected cells was extremely low (~0.01% of those in the RAAV group) at 2 hours post-infection, and then markedly elevated by 7,400-fold at day 3 (FIG. 25D). As expected, the transcription inhibitor actinomycin D largely abolished the mRNA elevation by AAV infection but had no effect on the mRNA elevation by RAAV infection (FIG. 33). Furthermore, the inventor found that both infections did not affect the expression of housekeeping mRNA for mGAPDH and DNA for 36B4 (30), confirming specific infection and gene expression induced by the viral vectors (FIG. 34). At the protein level, unlike AAV-Cre which expressed Cre persistently after infection, the expression of Cre in RAAV-infected cells could be detected as early as 17 hours post-infection and disappeared after one day (FIG. 25E). To express an equivalent amount of Cre at 17 hours post-infection, the viral genome copy number for RAAV (MOI=3,000 vg) was found to be about 10-fold of that for AAV (MOI=300 vg) (FIG. 25E), consistent with the Inventor's expectation that infected AAV-Cre could transcribe multiple copies of Cre mRNAs, leading to higher amounts of protein expressed. Furthermore, the RNAscope assay showed that RAAV entered the nucleus as early as 8 hours post-infection in Hela cells like conventional AAV (29) (FIG. 25F), and the nuclear entry of RAAV could be inhibited by bafilomycin A1 that interfered with viral infection (31) but not by actinomycin D that only affected mRNA transcription induced by AAV (FIG. 35). Notably, while the qPCR assay showed nRNAs remained detectable 5 days after RAAV infection, the expressed Cre protein disappeared by 1 day, suggesting termination of the expressed mRNAs. This is in line with the finding that although mRNA exit from the nucleus was observed as early as 8 hours post-infection, with increasing cytoplasmic aggregation of mRNAs afterward (possibly involving mRNA sequestration within P-bodies), a process known to downregulate translation (32) (FIGS. 25D and 25F). Taken together, the Inventor's findings showed that RAAV could mediate successful mRNA transfer to cultured cells and transiently express functional proteins, via trafficking to the nucleus similar to the conventional AAV. Thus, RAAV could also be used for introducing exogenous RNAs that regulate nuclear RNAs in the cell.

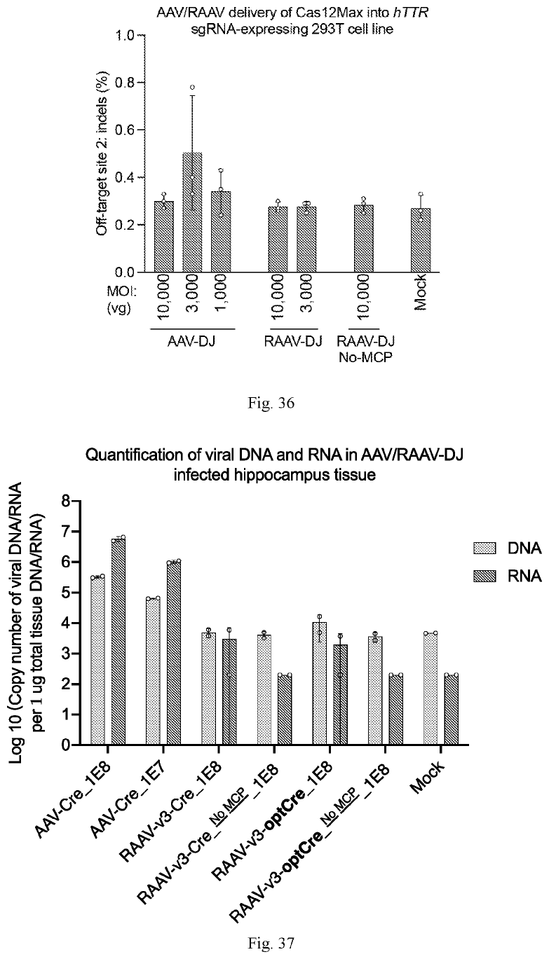

To expand the applicability of the RAAV system, the inventor examined whether RAAV-v3 could mediate the functional transfer of a large CRISPR-Cas12Max transcript (3774 nt), which exhibits editing activity comparable to Cas9 (33). The inventor incubated RAAV-v3-Cas12Max with 293T cells that constitutively express a guide RNA (gRNA) targeting hTTR (33) and analyzed gene editing efficacy 5 days after infection. The results showed that RAAVs were able to functionally transfer Cas12Max mRNAs, leading to 41.3±1.9% insertions and deletions (indels) at a MOI of 10,000 vg in recipient cells, with background-level editing at predicted off-target sites as that found in untreated cultured cells (FIGS. 25G and 25H, FIG. 36). Furthermore, at a similar level of on-target editing efficiency, the editing rate at the off-target site 1 of RAAV-Cas12Max (at MOI of 10,000 vg group) was 4.4-fold lower than that of AAV-Cas12Max (at MOI of 3,000 vg) (FIGS. 25G and 25H, FIG. 36). Finally, only trace amounts of viral vector DNA were detected in RAAV-Cas12Max-infected cells (FIG. 25I), greatly reducing the possibility of DNA insertional mutations (34). Overall, these results indicated that RAAV-mediated mRNA delivery provides a gene editing method with reduced off-target effects.

### Example 5: RAAV Delivery of RNAs Exhibits Tissue Tropism

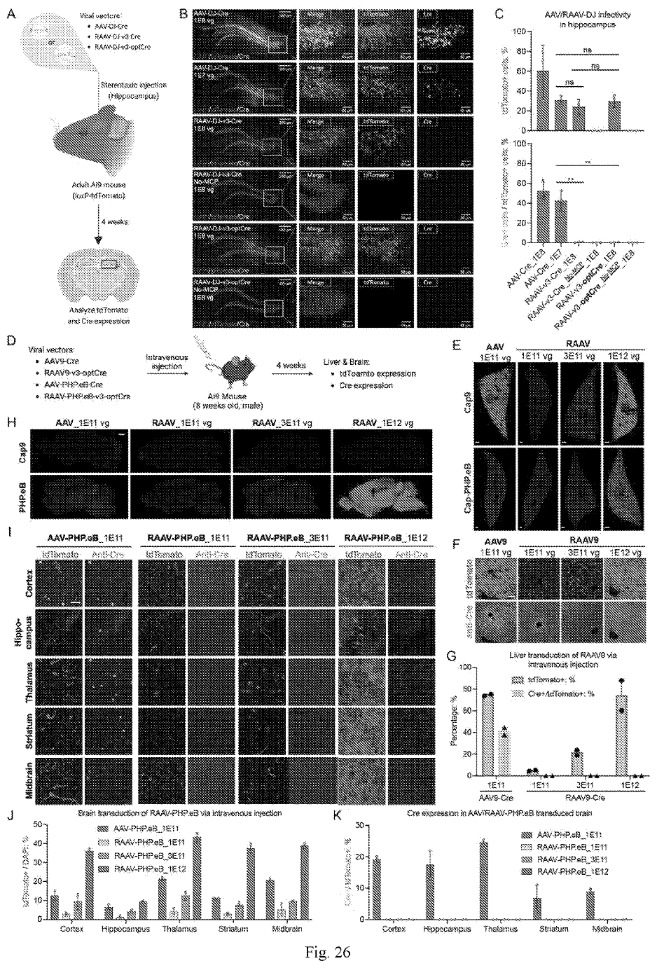

To assess the in vivo delivery efficacy of RAAVs, the inventor injected RAAV-v3-Cre, RAAV-v3-optCre, and AAV-Cre with capsid DJ into the hippocampus of adult Ai9 mice. Four weeks after injection, the inventor analyzed the hippocampal expression of tdTomato and Cre (FIG. 26A). The average percentage of tdTomato+ cells in hippocampi injected with RAAV-v3-Cre (24.2±7.1%, n=3) and RAAV-v3-optCre (29.9±6.0%) was lower than that observed in hippocampi injected with AAV-Cre at the same genome dose (1E8 vg/hippocampus) (60.2±24.0%), but was comparable to that of hippocampi injected with a lower dose (1E7 vg/hippocampus) of AAV-Cre (30.8±4.6%) (FIGS. 26B and 26C). In addition, Cre expression could only be detected in hippocampi infected with AAV-Cre (FIGS. 26B and 26C), whereas no viral vector-associated Cre DNA, RNA, or proteins was detected in RAAV-Cre-infection hippocampi (FIGS. 26B and 26C, FIG. 37). In comparison, no tdTomato+ or Cre+ cell was observed in the control mice treated with RAAV with no MCP (FIGS. 26B and 26C). Thus, RAAVs could efficiently deliver Cre mRNAs into the mouse hippocampus, and transiently express functional Cre protein.

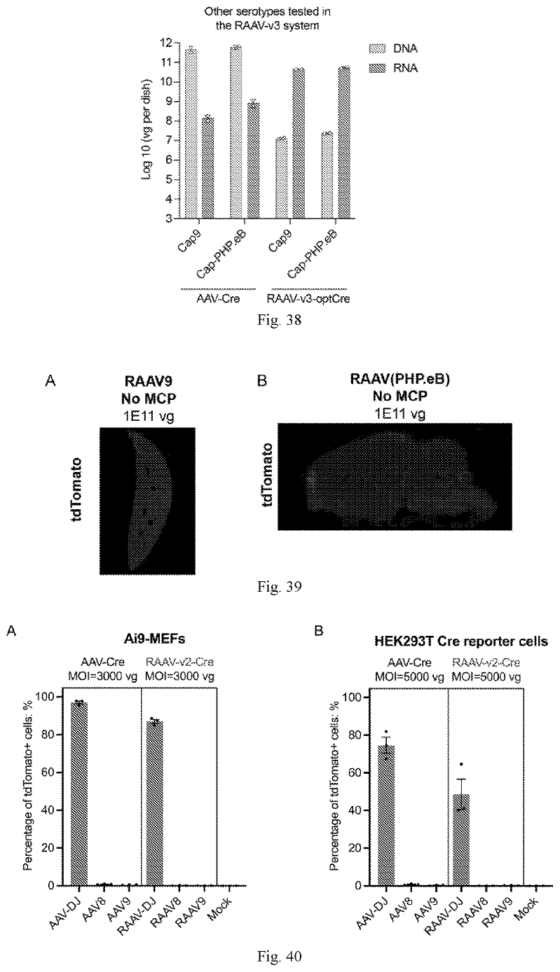

The AAV capsids isolated from various mammals (35) or engineered artificially (29) exhibit a wide variety of cell and tissue tropisms. Since the capsid is a major determinant of cell/tissue tropism of AAVs (35), the inventor next examined whether RAAV vectors could retain their original capsid tropism. Intravenously injected AAV with the capsid PHP.eB is known to exhibit higher infection efficiency in the brain and lower infection in the liver compared to AAV with capsid 9 (12, 36, 37). The inventor generated Cre-coding RAAVs and AAVs with either capsid PHP.eB or capsid 9 (FIG. 38) and intravenously injected each viral vector into the Cre reporter mouse Ai9. The mice were sacrificed 4 weeks after infection to analyze tdTomato and Cre expression in the liver and brain tissues (FIG. 26D). For capsid 9, the inventor found high percentages of tdTomato+ cells (74.7%) and Cre+ cells (41.3%) in the liver of AAV9-Cre injected mice (at a dose of 1E11 vg). In comparison, the percentage of tdTomato* cells in the liver of RAAV9-v3-optCre-injected mice was increased from 4.8% to 74%, as the vector dose increased from 1E11 vg to 1E12 vg, while no Cre+ cells were detected in the liver of RAAV9-v3-optCre infected mice as expected (FIG. 26E to 26G). In addition, both AAV9-Cre and RAAV9-v3-optCre yielded no obvious infection in the brain (FIG. 26H). As for the capsid PHP.eB, the inventor found much lower liver infection in AAV-PHP.eB-Cre and RAAV-PHP.eB-Cre infected mice compared with that of AAV9-Cre and RAAV9-Cre infected mice at the same dose, respectively (FIG. 26E). In contrast, the inventor found clear infection tropism for brain tissues, with high percentages of tdTomato+ cells in various brain tissues of AAV-PHP.eB-Cre-infected mice (12.6%, cortex; 6.4%, hippocampus; 21.4%, thalamus; 11.3%, striatum; 20.9%, midbrain), as compared to those found in AAV9-Cre-injected mice (at a dose of 1E11 vg) (FIG. 26H to 26J), consistent with previous studies (12, 36, 37). Interestingly, the inventor found that RAAV-PHP.eB-v3-optCre infected mice also effectively infected the whole brain, showing high percentage of tdTomato+ cells (36.1%, cortex; 9.4%, hippocampus; 43.4%, thalamus; 37.6%, striatum; 38.9%, midbrain) at a dose of 1E12 vg (FIG. 26H to 26J), indicating that RAAV-PHP.eB shares the same tropism with AAV-PHP.eB. However, due to the short lifetime of RAAV-PHP.eB-delivered mRNA and translated Cre, the inventor only observed Cre expression in AAV-PHP.eB-infected brains but not in RAAV-PHP.eB-infected brains (FIG. 26K). As controls, very few tdTomato+ cells were observed in the control mice treated with RAAV9 or RAAV-PHP.eB with no MCP (FIG. 39). In addition to the tissue tropism, RAAVs also retained their cellular tropisms, as shown by results using RAAV with various capsids to infect different types of culture cells (FIG. 40). Overall, these results indicated that RAAVs retained the infection tropism from their capsids and could mediate specific infection to target cells, tissues, and organs to transiently express functional proteins of interest.

In summary, by multi-step engineering, the inventor has developed a RNA delivery vector RAAV from the DNA virus AAV that exhibited high selectivity of RNA and very low residual DNA packaging (~0.005%). The RAAV systems combined the transient nature of RNA with a variety of tissue tropism of the AAV capsid, making them ideal for either broad-spectrum or tissue-specific RNA delivery. The results from this study demonstrate the feasibility of using rational engineering to change the virus genome type. Our strategy in developing RAAVs could be extended to other DNA viruses, in order to endow them the RNA packaging capability. Further optimization of the RAAV system of the disclosure could be achieved by improving the RNA-packaging specificity, vector productivity, translation efficiency, as well as the integrity and stability of packaged genome, especially for large-size RNAs. Nevertheless, the RAAV of the disclosure represents the first BBB-crossing RNA delivery system that could efficiently infect the whole brain, for basic neuroscience studies and therapeutic applications.

TABLE S1
List of 98 helicase-containing viral protein sequences used for alignment.

| Viral | | | | |
|---|---|---|---|---|
| genome | | | Accession | Accession |
| types | Abbreviations | Virus complete names | (GenBank) | (UniProt) |
| Single- | B19V (strain | Human parvovirus B19 (strain HV) | | Q9PZT1 |
| stranded | HV) | (HPV B19) | | |
| DNA | B19V | Human parvovirus B19 (HPV B19) | | Q6TV13 |
| (ssDNA) | AAV10 | Adeno-associated virus 10 | AY631965.1 | |
| | AAV11 | Adeno-associated virus 11 | AY631966.1 | |
| | AAV7 | Adeno-associated virus 7 | NC_006260.1 | |
| | AAV8 | Adeno-associated virus 8 | NC_006261.1 | |
| | AAV9 | Adeno-associated virus 9 | AY530579.1 | |
| | AAV1 | Adeno-associated virus 1 | NC_002077.1 | |
| | AAV6 | Adeno-associated virus 6 | AF028704.1 | |
| | AAV12 | Adeno-associated virus 12 | DQ813647.1 | |
| | AAV2 | Adeno-associated virus 2 | NC_001401.2 | |
| | AAV13 | Adeno-associated virus 13 | EU285562.1 | |
| | AAV4 | Adeno-associated virus 4 | NC_001829.1 | |
| | AAV3A | Adeno-associated virus 3 A | JB292182.1 | |
| | AAV3B | Adeno-associated virus 3 B | AF028705.1 | |
| | AAV5 | Adeno-associated virus 5 | NC_006152.1 | |
| | bPARV1 | Bovine parvovirus 1 | | Q2LD58 |
| | HBoV1 | Primate bocaparvovirus 1 (strain | | D0EZM8 |
| | | Human bocavirus 1 type 1) (HBoV1) | | |
| | | (Human bocavirus type 1) | | |
| | CPV-2 | Canine parvovirus type 2 (isolate | | P12929 |
| | | Dog/United States/CPV-N/1978) (CPV- | | |
| | | 2) | | |
| | HaPV | Hamster parvovirus H1 | | P03133 |
| | PPV | Porcine parvovirus (strain Kresse) | | P52502 |
| | | (PPV) | | |
| | AMDV | Aleutian mink disease parvovirus (strain | | P24030 |
| | | G) (ADV) | | |
| Single- | SINV-3 | Solenopsis invicta virus 3 (SINV-3) | | C1JCT1 |
| stranded | ABPV-R | Acute bee paralysis virus (strain | | Q9DSN9 |
| RNA | | Rothamsted) (ABPV) | | |
| (ssRNA) | CrPV | Cricket paralysis virus (isolate | | Q9IJX4 |
| | | Teleogryllus | | |
| | | commodus/Australia/CrPVVIC/1968) | | |
| | | (CrPV) | | |
| | DCV | Drosophila C virus (strain EB) (DCV) | | O36966 |
| | EoPV | Ectropis obliqua picorna-like virus | | Q6UP17 |
| | | (EoPV) | | |
| | BEC-NA1 | Bovine enteric calicivirus Newbury | | Q288N7 |
| | | agent-1 (isolate | | |
| | | Bovine/UK/Newbury1/1976) (BEC) | | |
| | BEC-NB | Bovine enteric calicivirus NB (isolate | | Q8JN60 |
| | | Bovine/United States/N ebraska/1980) | | |
| | | (BEC-NB) | | |
| | CaCV | Canine calicivirus (strain 48) (CaCV) | | Q8V736 |
| | FCV | Feline calicivirus (strain F9) (FCV) | | P27409 |
| | FCV-C | Feline calicivirus (strain Cat/United | | Q66914 |
| | | States/Urbana/1960) (FCV) | | |
| | SMSV-1 | San Miguel sea lion virus serotype 1 | | P36286 |
| | | (SMSV-1) (SMSV serotype 1) | | |
| | SMSV-4 | San Miguel sea lion virus serotype 4 | | P36287 |
| | | (SMSV-4) (SMSV serotype 4) | | |
| | VESV | Vesicular exanthema of swine virus | | Q9DUN3 |
| | | serotype A48 (isolate Swine/United | | |
| | | States/A48/1948) (VESV) | | |
| | PES | Porcine enteric sapovirus (isolate | | Q9QEJ5 |
| | | Swine/United States/Cowden/1980) | | |
| | | (Sw/SV/Cowden/1980/US) | | |
| | SaV-GII | Sapporo virus (isolate | | Q6XDK8 |
| | | GII/Human/Thailand/Mc10/2000) | | |
| | | (Hu/SaV/Mc10/2000/Thailand) | | |
| | SV | Sapporo virus (strain Human/United | | Q69014 |
| | | Kingdom/Manchester/1993) | | |
| | | (Hu/SV/Man/1993/UK) | | |
| | SaV-GI | Sapporo virus (isolate | | Q672I1 |
| | | GI/Human/Germany/pJG-Sap01) | | |
| | | (Hu/Dresden/pJG-Sap01/DE) | | |
| | EBHSV-GD | European brown hare syndrome virus | | Q96725 |
| | | (strain GD) | | |
| | | (Ha/LV/EBHSV/GD/1989/FR) | | |
| | | (EBHSV-GD) | | |
| | RHDV-V351 | Rabbit hemorrhagic disease virus (strain | | P27411 |
| | | V-351) (Ra/LV/RHDV/V351/1991/CK) | | |
| | | (RHDV-V351) | | |
| | RHDV- | Rabbit hemorrhagic disease virus (strain | | Q86119 |
| | AST89 | AST89) | | |
| | | (Ra/LV/RHDV/AST89/1989/SP) | | |
| | | (RHDV-AST89) | | |
| | LoV | Lordsdale virus (strain | | P54634 |
| | | GII/Human/United | | |
| | | Kingdom/Lordsdale/1993) (Human | | |
| | | enteric calicivirus) | | |
| | | (Hu/NV/LD/1993/UK) | | |
| | NV | Norwalk virus (strain GI/Human/United | | Q83883 |
| | | States/Norwalk/1968) | | |
| | | (Hu/NV/NV/1968/US) | | |
| | SHV | Southampton virus (strain | | Q04544 |
| | | GI/Human/United | | |
| | | Kingdom/Southampton/1991) (SHV) | | |
| | | (Hu/NV/SHV/1991/UK) | | |
| | PYFV | Parsnip yellow fleck virus (isolate P- | | Q05057 |
| | | 121) (PYFV) | | |
| | RTSV-A | Rice tungro spherical virus (strain A) | | Q83034 |
| | | (RTSV) (Rice tungro spherical | | |
| | | waikavirus) | | |
| | RTSV-V | Rice tungro spherical virus (strain Vt6) | | Q91PP5 |
| | | (RTSV) (Rice tungro spherical | | |
| | | waikavirus) | | |
| | AiV | Aichi virus (strain | | O91464 |
| | | Human/A846/88/1989) (AiV) (Aichi | | |
| | | virus (strain A846/88)) | | |
| | HKV-1 | Human klassevirus 1 (HKV-1) | | C6KEF6 |
| | SV-A | Salivirus A (isolate Human/Nigeria/NG- | | C5MSH2 |
| | | J1/2007) (SV-A) | | |
| | HPeV-1 | Human parechovirus 1 (strain Harris) | | Q66578 |
| | | (HPeV-1) (Echovirus 22) | | |
| | HPeV-5 | Human parechovirus 5 (strain CT86- | | Q9YID8 |
| | | 6760) (HPeV-5) (Echovirus 23) | | |
| | HPeV-2 | Human parechovirus 2 (strain | | O73556 |
| | | Williamson) (HPeV-2) (Echovirus 23) | | |
| | LV | Ljunganvirus 1 (LV) (Ljungan virus 1) | | Q8JV21 |
| | NoV | Nora virus | | Q27YG9 |
| | HAV-IA | Human hepatitis A virus genotype IA | | A3FMB2 |
| | | (isolate H2) (HHAV) (Human hepatitis | | |
| | | A virus (isolate | | |
| | | Human/China/H2/1982)) | | |
| | HHAV | Human hepatitis A virus genotype IB | | P13901 |
| | | (isolate MBB) (HHAV) (Human | | |
| | | hepatitis A virus (isolate | | |
| | | Human/Northern Africa/MBB/1978)) | | |
| | SHAV | Simian hepatitis A virus genotype V | | P14553 |
| | | (isolate AGM-27) (SHAV) (Simian | | |
| | | hepatitis A virus (isolate | | |
| | | Cercopithecus/Kenya/AGM-27/1985)) | | |
| | AEV-CV | Avian encephalomyelitis virus (strain | | Q9YLS4 |
| | | Calnek vaccine) (AEV) | | |
| | AEV-L | Avian encephalomyelitis virus (strain | | Q6WQ42 |
| | | L2Z) (AEV) | | |
| | CosV | Cosavirus A (isolate | | B8XTP8 |
| | | Human/Pakistan/0553/—) (HCoSV-A) | | |
| | SVV | Seneca Valley virus (isolate —/United | | Q155Z9 |
| | | States/SSV-001/2002) (SVV) | | |
| | EMCV | Encephalomyocarditis virus | | P03304 |
| | MEMCV | Mengo encephalomyocarditis virus | | P12296 |
| | SaFV | Saffold virus (SafV) (Human TMEV- | | C0MHL9 |
| | | like virus-Saffold) | | |
| | TMEV-B | Theiler's murine encephalomyelitis | | P08544 |
| | | virus (strain BeAn 8386) (TMEV) | | |
| | TMEV-G | Theiler's murine encephalomyelitis | | P08545 |
| | | virus (strain GDVII) (TMEV) | | |
| | FMDV-S | Foot-and-mouth disease virus (isolate —/ | | P03311 |
| | | Spain/S8c1SantaPau/1970 serotype C) | | |
| | | (FMDV) | | |
| | FMDV-G | Foot-and-mouth disease virus (isolate —/ | | P15072 |
| | | Germany/C1Oberbayen/1960 serotype | | |
| | | C) (FMDV) | | |
| | FMDV-A | Foot-and-mouth disease virus (isolate —/ | | P49303 |
| | | Azerbaijan/A22-550/1965 serotype A) | | |
| | | (FMDV) | | |
| | HRV-1A | Human rhinovirus 1A (HRV-1A) | | P23008 |
| | HRV-16 | Human rhinovirus 16 (HRV-16) | | Q82122 |
| | HRV2 | Human rhinovirus 2 (HRV-2) | | P04936 |
| | HRV-89 | Human rhinovirus A serotype 89 (strain | | P07210 |
| | | 41467-Gallo) (HRV-89) | | |
| | HRV-14 | Human rhinovirus 14 (HRV-14) | | P03303 |
| | HRV-3 | Human rhinovirus 3 (HRV-3) | | Q82081 |
| | BEV | Bovine enterovirus (strain VG-5-27) | | P12915 |
| | | (BEV) | | |
| | PEV-9 | Porcine enterovirus 9 (strain | | O41174 |
| | | UKG/410/73) | | |
| | EV-68 | Human enterovirus D68 (EV68) (EV- | | Q68T42 |
| | | 68) | | |
| | EV-70 | Human enterovirus 70 (strain J670/71) | | P32537 |
| | | (EV70) (EV-70) | | |
| | EV-71 | Human enterovirus 71 (EV71) (EV-71) | | B9VUU3 |
| | Cox A16 | Coxsackievirus A16 (strain G-10) | | Q65900 |
| | EV-71M | Human enterovirus 71 (strain | | Q66479 |
| | | 7423/MS/87) (EV71) (EV-71) | | |
| | CV-A21 | Coxsackievirus A21 (strain Coe) | | P22055 |
| | CV-A24 | Coxsackievirus A24 (strain EH24/70) | | P36290 |
| | PV-1 | Poliovirus type 1 (strain Mahoney) | | P03300 |
| | PV | Human poliovirus 2 | | Q4QZ09 |
| | E6-C | Echovirus 6 (strain Charles) | | Q66474 |
| | SVDV | Swine vesicular disease virus (strain | | P16604 |
| | | H/3 ′76) (SVDV) | | |
| | CB5 | Coxsackievirus B5 (strain Peterborough/ | | Q03053 |
| | | 1954/UK/85) | | |
| | ECV12 | Echovirus 12 (strain Travis) | | Q66575 |
| | ECV11 | Echovirus 11 (strain Gregory) | | P29813 |
| | CAV-9 | Coxsackievirus A9 (strain Griggs) | | P21404 |
| | CAV-B3 | Coxsackievirus B3 (strain Woodruff) | | Q66282 |
| | CAV-B4 | Coxsackievirus B4 (strain E2) | | Q86887 |
| | E9 | Echovirus 9 (strain Hill) | | Q66849 |
| | E30 | Echovirus 30 (strain Bastianni) | | Q9WN78 |

TABLE S2
List of single helicase mutations.

| | Mutation | | | |
|---|---|---|---|---|
| | description | | | |
| | (Mutation | RNA | | |
| | site in AAV2 | Packaging | Sequences used for alignment- | Alignment |
| Mutants | Rep78) | Efficiency | Alignment tools | results |
| M1 | G325P | 1.46330 | Helicase core sequence- | FIG. 27B/FIG. 27C |
| | | | AlignX/MUSCLE | |
| M2 | G325I | 0.04546 | Helicase core sequence- | FIG. 27B/FIG. 27C |
| | | | AlignX/MUSCLE | |
| M3 | K326E | 0.00940 | Helicase core sequence- | FIG. 27B/FIG. 27C |
| | | | AlignX/MUSCLE | |
| M4 | K326R | 0.52683 | Helicase core sequence- | FIG. 27B/FIG. 27C |
| | | | AlignX/MUSCLE | |
| M5 | R327P | 1.39031 | Helicase core sequence- | FIG. 27B/FIG. 27C |
| | | | AlignX/MUSCLE | |
| M6 | N328V | 0.70942 | Helicase core sequence- | FIG. 27B/FIG. 27C |
| | | | AlignX/MUSCLE | |
| M7 | W331I | 1.40986 | Helicase core sequence- | FIG. 27B/FIG. 27C |
| | | | AlignX/MUSCLE | |
| M8 | F333H | 0.82036 | Helicase core sequence- | FIG. 27B/FIG. 27C |
| | | | AlignX/MUSCLE | |
| M9 | F333Y | 0.75441 | Helicase core sequence- | FIG. 27B/FIG. 27C |
| | | | AlignX/MUSCLE | |
| M10 | F333K | 0.17432 | Helicase core sequence- | FIG. 27B/FIG. 27C |
| | | | AlignX/MUSCLE | |
| M11 | P335S | 0.41111 | Helicase core sequence- | FIG. 27B/FIG. 27C |
| | | | AlignX/MUSCLE | |
| M12 | A336P | 3.65482 | Helicase core sequence- | FIG. 27B/FIG. 27C |
| | | | AlignX/MUSCLE | |
| M13 | A336S | 2.61469 | Helicase core sequence- | FIG. 27B/FIG. 27C |
| | | | AlignX/MUSCLE | |
| M14 | A336R | 1.23847 | Helicase core sequence- | FIG. 27B/FIG. 27C |
| | | | AlignX/MUSCLE | |
| M15 | T337G | 2.17237 | Helicase core sequence- | FIG. 27B/FIG. 27C |
| | | | AlignX/MUSCLE | |
| M16 | T338G | 0.02057 | Helicase core sequence- | FIG. 27B/FIG. 27C |
| | | | AlignX/MUSCLE | |
| M17 | T341S | 0.42000 | Helicase core sequence- | FIG. 27B/FIG. 27C |
| | | | AlignX/MUSCLE | |
| M18 | N342I | 0.16545 | Helicase core sequence- | FIG. 27B/FIG. 27C |
| | | | AlignX/MUSCLE | |
| M19 | I343T | 4.29701 | Helicase core sequence- | FIG. 27B/FIG. 27C |
| | | | AlignX/MUSCLE | |
| M20 | I343A | 1.84896 | Helicase core sequence- | FIG. 27B/FIG. 27C |
| | | | AlignX/MUSCLE | |
| M21 | I343L | 1.17213 | Helicase core sequence- | FIG. 27B/FIG. 27C |
| | | | AlignX/MUSCLE | |
| M22 | A344T | 4.22375 | Helicase core sequence- | FIG. 27B/FIG. 27C |
| | | | AlignX/MUSCLE | |
| M23 | A344V | 4.64451 | Helicase core sequence- | FIG. 27B/FIG. 27C |
| | | | AlignX/MUSCLE | |
| M24 | A344S | 0.66657 | Helicase core sequence- | FIG. 27B/FIG. 27C |
| | | | AlignX/MUSCLE | |
| M25 | E345N | 0.03140 | Helicase core sequence- | FIG. 27B/FIG. 27C |
| | | | AlignX/MUSCLE | |
| M26 | A346F | 0.02491 | Helicase core sequence- | FIG. 27B/FIG. 27C |
| | | | AlignX/MUSCLE | |
| M27 | H349K | 0.13027 | Helicase core sequence- | FIG. 27B/FIG. 27C |
| | | | AlignX/MUSCLE | |
| M28 | P352T | 0.78490 | Helicase core sequence- | FIG. 27B/FIG. 27C |
| | | | AlignX/MUSCLE | |
| M29 | P365Y | 0.10346 | Helicase core sequence- | FIG. 27B/FIG. 27C |
| | | | AlignX/MUSCLE | |
| M30 | N367D | 0.01121 | Helicase core sequence- | FIG. 27B/FIG. 27C |
| | | | AlignX/MUSCLE | |
| M31 | D368G | 0.01026 | Helicase core sequence- | FIG. 27B/FIG. 27C |
| | | | AlignX/MUSCLE | |
| M32 | C369Y | 0.01311 | Helicase core sequence- | FIG. 27B/FIG. 27C |
| | | | AlignX/MUSCLE | |
| M33 | V370K | 0.99370 | Helicase core sequence- | FIG. 27B/FIG. 27C |
| | | | AlignX/MUSCLE | |
| M34 | D371Q | 1.13495 | Helicase core sequence- | FIG. 27B/FIG. 27C |
| | | | AlignX/MUSCLE | |
| M35 | D371G | 0.28173 | Helicase core sequence- | FIG. 27B/FIG. 27C |
| | | | AlignX/MUSCLE | |
| M36 | D371N | 0.37067 | Helicase core sequence- | FIG. 27B/FIG. 27C |
| | | | AlignX/MUSCLE | |
| M37 | K372Q | 3.42519 | Helicase core sequence- | FIG. 27B/FIG. 27C |
| | | | AlignX/MUSCLE | |
| M38 | K372E | 1.24679 | Helicase core sequence- | FIG. 27B/FIG. 27C |
| | | | AlignX/MUSCLE | |
| M39 | K372N | 2.38293 | Helicase core sequence- | FIG. 27B/FIG. 27C |
| | | | AlignX/MUSCLE | |
| M40 | M373S | 1.04496 | Helicase core sequence- | FIG. 27B/FIG. 27C |
| | | | AlignX/MUSCLE | |
| M41 | M373E | 0.54895 | Helicase core sequence- | FIG. 27B/FIG. 27C |
| | | | AlignX/MUSCLE | |
| M42 | M373A | 0.91848 | Helicase core sequence- | FIG. 27B/FIG. 27C |
| | | | AlignX/MUSCLE | |
| M43 | I375V | 1.05809 | Helicase core sequence- | FIG. 27B/FIG. 27C |
| | | | AlignX/MUSCLE | |
| M44 | W376I | 0.00814 | Helicase core sequence- | FIG. 27B/FIG. 27C |
| | | | AlignX/MUSCLE | |
| M45 | W377M | 0.02543 | Helicase core sequence- | FIG. 27B/FIG. 27C |
| | | | AlignX/MUSCLE | |
| M46 | E378D | 3.23780 | Helicase core sequence- | FIG. 27B/FIG. 27C |
| | | | AlignX/MUSCLE | |
| M47 | E379D | 0.00438 | Helicase core sequence- | FIG. 27B/FIG. 27C |
| | | | AlignX/MUSCLE | |
| M48 | G380L | 0.01503 | Helicase core sequence- | FIG. 27B/FIG. 27C |
| | | | AlignX/MUSCLE | |
| M49 | G380F | 0.02050 | Helicase core sequence- | FIG. 27B/FIG. 27C |
| | | | AlignX/MUSCLE | |
| M50 | C405H | 1.01224 | Helicase core sequence- | FIG. 27B/FIG. 27C |
| | | | AlignX/MUSCLE | |
| M51 | K406R | 0.60638 | Helicase core sequence- | FIG. 27B/FIG. 27C |
| | | | AlignX/MUSCLE | |
| M52 | T419S | 1.45999 | Helicase core sequence- | FIG. 27B/FIG. 27C |
| | | | AlignX/MUSCLE | |
| M53 | T419F | 0.02221 | Helicase core sequence- | FIG. 27B/FIG. 27C |
| | | | AlignX/MUSCLE | |
| M54 | T419I | 0.78724 | Helicase core sequence-MUSCLE | FIG. 27C |
| M55 | T419L | 0.83450 | Helicase core sequence-MUSCLE | FIG. 27C |
| M56 | S420A | 2.42642 | Helicase core sequence- | FIG. 27B/FIG. 27C |
| | | | AlignX/MUSCLE | |
| M57 | S420M | 0.01624 | Helicase core sequence-MUSCLE | FIG. 27C |
| M58 | S420C | 0.13999 | Helicase core sequence-MUSCLE | FIG. 27C |
| M59 | N421T | 0.01190 | Helicase core sequence-MUSCLE | FIG. 27C |
| M60 | N421S | 0.01571 | Helicase core sequence- | FIG. 27B/FIG. 27C |
| | | | AlignX/MUSCLE | |
| M61 | T422H | 2.37646 | Helicase core sequence- | FIG. 27B/FIG. 27C |
| | | | AlignX/MUSCLE | |
| M62 | T422S | 1.35068 | Helicase core sequence-MUSCLE | FIG. 27C |
| M63 | M424N | 0.11135 | Helicase core sequence-MUSCLE | FIG. 27C |
| M64 | C425I | 3.07812 | Helicase core sequence-AlignX | FIG. 27B |
| M65 | Q442K | 0.50973 | Helicase core sequence-MUSCLE | FIG. 27C |
| M66 | Q442F | 0.40881 | Helicase core sequence- | FIG. 27B/FIG. 27C |
| | | | AlignX/MUSCLE | |
| M67 | Q442H | 1.69419 | Helicase core sequence-MUSCLE | FIG. 27C |
| M68 | Q442R | 1.32131 | Helicase core sequence-MUSCLE | FIG. 27C |
| M69 | Q442L | 0.19391 | Helicase core sequence-AlignX | FIG. 27B |
| M70 | Q442V | 0.29877 | Helicase core sequence-AlignX | FIG. 27B |
| M71 | D443S | 3.05550 | Helicase core sequence-AlignX | FIG. 27B |
| M72 | D443R | 0.01460 | Helicase core sequence- | FIG. 27B/FIG. 27C |
| | | | AlignX/MUSCLE | |
| M73 | D443Y | 1.77945 | Helicase core sequence-AlignX | FIG. 27B |
| M74 | D443N | 2.86941 | Helicase core sequence- | FIG. 27B/FIG. 27C |
| | | | AlignX/MUSCLE | |
| M75 | D443A | 2.76411 | Helicase core sequence- | FIG. 27B/FIG. 27C |
| | | | AlignX/MUSCLE | |
| M76 | M445I | 2.16563 | Helicase core sequence-MUSCLE | FIG. 27C |
| M77 | M445R | 0.03985 | Helicase core sequence-AlignX | FIG. 27B |
| M78 | M445F | 0.08321 | Helicase core sequence-MUSCLE | FIG. 27C |
| M79 | F446R | 0.26141 | Helicase core sequence-AlignX | FIG. 27B |
| M80 | F446I | 0.74098 | Helicase core sequence- | FIG. 27B/FIG. 27C |
| | | | AlignX/MUSCLE | |
| M81 | K447F | 4.16352 | Helicase core sequence- | FIG. 27B/FIG. 27C |
| | | | AlignX/MUSCLE | |
| M82 | K447N | 1.38774 | Helicase core sequence- | FIG. 27B/FIG. 27C |
| | | | AlignX/MUSCLE | |
| M83 | K447H | 0.32861 | Helicase core sequence- | FIG. 27B/FIG. 27C |
| | | | AlignX/MUSCLE | |
| M84 | K447P | 0.09325 | Helicase core sequence- | FIG. 27B/FIG. 27C |
| | | | AlignX/MUSCLE | |
| M85 | K447T | 1.05156 | Helicase core sequence- | FIG. 27B/FIG. 27C |
| | | | AlignX/MUSCLE | |
| M86 | K447A | 0.28676 | Helicase core sequence- | FIG. 27B/FIG. 27C |
| | | | AlignX/MUSCLE | |
| M87 | E449D | 3.72501 | Helicase core sequence- | FIG. 27B/FIG. 27C |
| | | | AlignX/MUSCLE | |
| M88 | E449I | 0.18097 | Helicase core sequence- | FIG. 27B/FIG. 27C |
| | | | AlignX/MUSCLE | |
| M89 | E449R | 0.08135 | Helicase core sequence- | FIG. 27B/FIG. 27C |
| | | | AlignX/MUSCLE | |
| M90 | L450M | 3.47444 | Helicase core sequence- | FIG. 27B/FIG. |
| | | | AlignX/MUSCLE | 27C/FIG. 27D |
| | | | complete viral protein sequence | |
| | | | (harboring an SF3 helicase)-AlignX | |
| M91 | L450I | 1.23088 | Helicase core sequence- | FIG. 27B/FIG. |
| | | | AlignX/MUSCLE | 27C/FIG. 27D |
| | | | complete viral protein sequence | |
| | | | (harboring an SF3 helicase)-AlignX | |
| M92 | L450V | 1.90031 | Helicase core sequence- | FIG. 27B/FIG. 27C |
| | | | AlignX/MUSCLE | |
| M93 | T451D | 2.86526 | Helicase core sequence- | FIG. 27B/FIG. |
| | | | AlignX/MUSCLE | 27C/FIG. 27D |
| | | | complete viral protein sequence | |
| | | | (harboring an SF3 helicase)-AlignX | |
| M94 | T451I | 0.65118 | Helicase core sequence-MUSCLE | FIG. 27C |
| M95 | T451E | 1.23498 | Helicase core sequence- | FIG. 27B/FIG. 27C |
| | | | AlignX/MUSCLE | |
| M96 | T451K | 0.22213 | Helicase core sequence- | FIG. 27B/FIG. 27C |
| | | | AlignX/MUSCLE | |
| M97 | T451N | 0.88349 | Helicase core sequence- | FIG. 27B/FIG. |
| | | | AlignX/MUSCLE | 27C/FIG. 27D |
| | | | complete viral protein sequence | |
| | | | (harboring an SF3 helicase)-AlignX | |
| M98 | L454F | 3.47694 | complete viral protein sequence | FIG. 27D |
| | | | (harboring an SF3 helicase)-AlignX | |
| M99 | L454V | 0.97041 | Helicase core sequence- | FIG. 27B/FIG. 27C |
| | | | AlignX/MUSCLE | |
| M100 | D455F | 1.81295 | complete viral protein sequence | FIG. 27D |
| | | | (harboring an SF3 helicase)-AlignX | |
| M101 | D455K | 0.27878 | Helicase core sequence- | FIG. 27B/FIG. 27C |
| | | | AlignX/MUSCLE | |
| M102 | D455H | 0.22192 | Helicase core sequence- | FIG. 27B/FIG. 27C |
| | | | AlignX/MUSCLE | |
| M103 | D455Y | 1.67406 | complete viral protein sequence | FIG. 27D |
| | | | (harboring an SF3 helicase)-AlignX | |
| M104 | D455T | 2.25634 | Helicase core sequence- | FIG. 27B/FIG. 27C |
| | | | AlignX/MUSCLE | |
| M105 | D455M | 1.45338 | Helicase core sequence- | FIG. 27B/FIG. 27C |
| | | | AlignX/MUSCLE | |
| M106 | H456F | 0.66235 | complete viral protein sequence | FIG. 27D |
| | | | (harboring an SF3 helicase)-AlignX | |
| M107 | H456D | 3.87216 | Helicase core sequence- | FIG. 27B/FIG. 27C |
| | | | AlignX/MUSCLE | |
| M108 | H456S | 1.66982 | Helicase core sequence- | FIG. 27B/FIG. 27C |
| | | | AlignX/MUSCLE | |
| M109 | D457E | 2.55194 | Helicase core sequence- | FIG. 27B/FIG. 27C |
| | | | AlignX/MUSCLE | |
| M110 | D457S | 1.38270 | Helicase core sequence- | FIG. 27B/FIG. 27C |
| | | | AlignX/MUSCLE | |
| M111 | D457F | 1.75484 | Helicase core sequence-MUSCLE | FIG. 27C |
| M112 | F458Y | 4.02989 | Helicase core sequence- | FIG. 27B/FIG. 27C |
| | | | AlignX/MUSCLE | |
| M113 | F458K | 0.01841 | Helicase core sequence- | FIG. 27B/FIG. 27C |
| | | | AlignX/MUSCLE | |
| M114 | V461L | 4.21838 | Rational design (DOI: | |
| | | | 10.1371/journal.ppat.1007304) | |

TABLE S3
List of combined helicase mutations.

| | Mutation description (Mutation sites in | RNA Packaging | Combination |
|---|---|---|---|
| Mutants | AAV2 Rep78) | Efficiency | method |
| M115 | R327P + A336P + T337G | 0.36421 | A + A |
| M116 | A336P + T337G | 1.59983 | A + A |
| M117 | I343T + A344T | 0.01608 | A + A |
| M118 | K372Q + E378D | 1.21999 | B + B |
| M119 | D443S + M445I | 1.32532 | R + R |
| M120 | D443S + L454F + D455F | 1.05394 | R + R |
| M121 | K447F + E449D | 0.86355 | R + R |
| M122 | K447F + E449D + T451D | 2.37624 | R + R |
| M123 | K447F + L450M | 1.71678 | R + R |
| M124 | K447F + F458Y | 1.39142 | R + R |
| M125 | K447F + H456D + F458Y | 2.05314 | R + R |
| M126 | K447F + E449D + H456D + F458Y | 0.56130 | R + R |
| M127 | K447F + V461L | 1.40225 | R + R |
| M128 | E449D + L450M | 1.88761 | R + R |
| M129 | L450M + T451D | 1.27433 | R + R |
| M130 | L450M + L454F + D455F | 0.37186 | R + R |
| M131 | L450M + V461L | 0.75258 | R + R |
| M132 | L454F + D455F | 4.11069 | R + R |
| M133 | L454F + D455F + V461L | 0.00542 | R + R |
| M134 | D455T + H456D + D457E + F458Y | 1.23872 | R + R |
| M135 | D455T + H456D + D457E + F458Y + V461L | 0.00352 | R + R |
| M136 | H456D + D457E | 0.55666 | R + R |
| M137 | H456D + D457E + F458Y | 3.08094 | R + R |
| M138 | H456D + F458Y | 0.67510 | R + R |
| M139 | F458Y + V461L | 1.24407 | R + R |
| M140 | R327P + E378D | 0.00011 | A + B |
| M141 | A344T + K372Q | 3.80808 | A + B |
| M142 | A344T + E378D | 0.45666 | A + B |
| M143 | A336P + A344T + K447F | 1.42594 | A + R |
| M144 | A336P + A344V + K447F | 2.15909 | A + R |
| M145 | A336P + I343T + K447F | 0.03361 | A + R |
| M146 | I343T + D443N + K447F | 0.02407 | A + R |
| M147 | I343T + K447F | 5.96797 | A + R |
| M148 | I343T + L450M | 1.07929 | A + R |
| M149 | I343T + K447F + E449D + T451D | 0.40626 | A + R |
| M150 | I343T + K447F + E449D + L450M + T451D | 0.01005 | A + R |
| M151 | A344T + D443N + K447F | 0.17932 | A + R |
| M152 | A344T + K447F | 8.47876 | A + R |
| M153 | A344V + D443N + K447F | 0.01145 | A + R |
| M154 | A344V + K447F | 12.45554 | A + R |
| M155 | A344T + LA50M | 5.49697 | A + R |
| M156 | A344V + L450M | 10.48506 | A + R |
| M157 | A344T + K447F + E449D + T451D | 1.62286 | A + R |
| M158 | A344T + K447F + E449D + L450M + T451D | 0.06964 | A + R |
| M159 | A344T + LA54F + D455F | 0.00300 | A + R |
| M160 | A344T + V461L | 0.14733 | A + R |
| M161 | K372Q + K447F | 2.34934 | B + R |
| M162 | K372Q + L450M | 2.05281 | B + R |
| M163 | K372Q ++ K447F + E449D + T451D | 2.05715 | B + R |
| M164 | K372Q + V461L | 1.95938 | B + R |
| M165 | E378D + M445I | 0.50391 | B + R |
| M166 | E378D + L454F | 0.43972 | B + R |
| M167 | E378D + L454F + D455F | 0.01513 | B + R |
| M168 | E378D + K447F | 2.53588 | B + R |
| M169 | E378D + LA50M | 1.30844 | B + R |
| M170 | E378D ++ K447F + E449D + T451D | 1.08150 | B + R |
| M171 | E378D + V461L | 0.49693 | B + R |
| M172 | R327P + E378D + L454F | 0.00004 | A + B + R |
| M173 | R327P + E378D + L454F + D455F | 0.00006 | A + B + R |
| M174 | I343T + K372Q + K447F | 0.10181 | A + B + R |
| M175 | I343T + K372Q + L450M | 0.01858 | A + B + R |
| M176 | A344T + K372Q + K447F | 1.19473 | A + B + R |
| M177 | A344V + K372Q + K447F | 2.08465 | A + B + R |
| M178 | A344T + K372Q + L450M | 0.02895 | A + B + R |
| M179 | A344V + K372Q + L450M | 0.08780 | A + B + R |
| M180 | I343T + S420A + K447F | 0.36259 | A + C + R |
| M181 | A344T + S420A + K447F | 0.01664 | A + C + R |
| M182 | A344V + S420A + K447F | 0.01091 | A + C + R |
| M23 | A344V | 3.50500 | NA |
| M37 | K372Q | 2.76686 | NA |
| M81 | K447F | 2.77086 | NA |
| M90 | L450M | 3.14696 | NA |
| M98 | L454F | 3.40978 | NA |

TABLE S4
Optimized Cre coding sequences.

| Name | Sequences | Online tools |
|---|---|---|
| Cre opt1 | ATGCCTAAAAAGAAAAGGAAGGTGAGTAATTTGCTTA | https://sg.idtdna. |
| | CAGTGCATCAGAACCTTCCAGCATTGCCAGTAGATGCC | com/CodonOpt |
| | ACCTCCGATGAGGTAAGGAAAAACCTCATGGACATGT | |
| | TCCGGGATCGGCAAGCATTTTCAGAACATACCTGGAA | |
| | AATGCTTCTTTCAGTTTGCCGGAGTTGGGCAGCATGGT | |
| | GTAAACTCAATAATAGGAAGTGGTTTCCTGCTGAGCCC | |
| | GAGGACGTGCGAGACTATCTCCTTTATCTGCAGGCAAG | |
| | GGGGCTTGCAGTTAAAACTATACAACAACACCTCGGC | |
| | CAGCTCAATATGCTTCACCGGCGCAGTGGGCTGCCCCG | |
| | CCCAAGCGATTCTAACGCAGTGTCACTCGTTATGCGCA | |
| | GGATTCGGAAGGAGAACGTGGATGCAGGTGAGCGCGC | |
| | TAAACAAGCACTGGCTTTCGAAAGGACTGACTTTGAC | |
| | CAGGTCAGGTCCCTGATGGAAAACTCAGACAGATGCC | |
| | AAGACATCAGAAATCTTGCCTTTCTTGGCATTGCCTAC | |
| | AATACATTGCTTCGGATTGCCGAAATAGCACGCATAAG | |
| | GGTTAAAGACATTAGTAGGACAGATGGGGGACGCATG | |
| | CTCATCCATATAGGAAGGACTAAGACCCTCGTCAGCAC | |
| | CGCTGGCGTTGAGAAAGCCTTGTCCTTGGGCGTCACA | |
| | AAGCTCGTCGAACGGTGGATCAGTGTCAGCGGAGTCG | |
| | CCGACGATCCAAATAACTACCTCTTCTGCAGGGTGCGA | |
| | AAAAATGGCGTCGCCGCACCAAGTGCTACTTCACAAT | |
| | TGAGTACCAGAGCTCTTGAGGGGATTTTCGAGGCTACT | |
| | CATCGGCTTATATACGGAGCCAAGGATGATAGCGGTCA | |
| | GAGATACTTGGCATGGTCTGGTCACAGCGCTAGAGTG | |
| | GGTGCCGCACGGGACATGGCACGCGCTGGAGTGAGCA | |
| | TACCTGAAATTATGCAAGCAGGCGGCTGGACTAATGTG | |
| | AATATTGTTATGAATTATATCCGCAATCTCGACTCCGAA | |
| | ACCGGAGCCATGGTACGCCTGCTCGAGGACGGTGACT | |
| | AA | |
| Cre opt2 | ATGCCCAAAAAGAAGAGGAAAGTAAGTAATTTGCTTA | https://sg.idtdna. |
| | CCGTCCATCAGAATCTCCCCGCACTTCCCGTTGATGCT | com/CodonOpt |
| | ACTTCCGATGAGGTTAGAAAGAACCTGATGGATATGTT | |
| | CCGGGACCGCCAGGCTTTTTCCGAGCATACTTGGAAG | |
| | ATGTTGCTTTCCGTGTGCCGGAGCTGGGCAGCGTGGT | |
| | GTAAGCTTAACAATAGAAAATGGTTTCCTGCGGAACCC | |
| | GAGGATGTGAGAGATTATCTTCTTTACTTGCAGGCACG | |
| | AGGCCTCGCCGTGAAGACGATTCAGCAACACTTGGGC | |
| | CAACTTAATATGCTCCATCGCAGATCCGGGTTGCCTCG | |
| | ACCGAGTGACTCTAATGCAGTCTCACTTGTGATGCGAC | |
| | GAATCCGAAAGGAAAATGTTGACGCCGGTGAACGAGC | |
| | CAAACAAGCACTCGCATTCGAACGCACCGACTTCGAC | |
| | CAAGTCAGGAGCCTCATGGAAAATTCAGACCGCTGTC | |
| | AAGATATCCGAAACCTTGCCTTTCTGGGCATAGCGTAT | |
| | AACACATTGTTGAGGATAGCAGAAATAGCGAGGATTA | |
| | GGGTTAAGGACATCTCTAGGACCGATGGAGGTAGGAT | |
| | GTTGATCCACATCGGCCGCACAAAAACACTCGTTAGC | |
| | ACGGCAGGGGTAGAAAAGGCACTTTCTCTTGGAGTAA | |
| | CGAAACTTGTTGAACGATGGATTTCTGTGTCTGGAGTC | |
| | GCAGATGACCCGAATAATTACCTCTTCTGCCGAGTTAG | |
| | GAAAAACGGCGTCGCCGCGCCGTCTGCTACAAGTCAA | |
| | CTTTCTACAAGAGCACTTGAGGGCATTTTCGAAGCAA | |
| | CGCATCGACTGATTTATGGAGCGAAGGACGATTCTGGC | |
| | CAGCGATATCTCGCGTGGTCCGGCCACAGTGCTCGCGT | |
| | CGGGGCAGCGCGGGATATGGCGCGCGCAGGAGTAAGT | |
| | ATACCGGAAATCATGCAAGCAGGTGGCTGGACGAACG | |
| | TTAATATCGTGATGAACTATATTAGGAACCTGGATAGTG | |
| | AAACTGGAGCGATGGTCCGCTTGTTGGAAGACGGAGA | |
| | CTAG | |
| Cre opt3 | ATGCCTAAGAAGAAGAGGAAGGTGAGCAACCTGCTG | https://clims4. |
| | ACCGTGCATCAGAACCTGCCTGCCCTGCCTGTGGACG | genewiz.com/ |
| | CCACAAGCGACGAGGTTAGGAAGAATTTAATGGACAT | Toolbox/ |
| | GTTTAGGGATAGGCAAGCCTTCAGCGAGCACACCTGG | CodonOptimization |
| | AAGATGCTGCTGAGCGTGTGTAGGAGCTGGGCCGCCT | |
| | GGTGCAAGCTGAACAATAGGAAGTGGTTCCCTGCCGA | |
| | GCCTGAGGACGTGAGGGACTACCTGCTGTACCTGCAA | |
| | GCTAGGGGCCTGGCCGTGAAGACCATTCAGCAGCACC | |
| | TGGGACAGCTGAACATGCTGCATAGGAGGAGCGGCCT | |
| | GCCTAGGCCTAGCGACAGCAACGCCGTGAGCCTGGTG | |
| | ATGAGGAGGATTAGGAAGGAGAACGTGGACGCCGGC | |
| | GAGAGGGCCAAGCAAGCCCTGGCCTTCGAGAGGACC | |
| | GACTTCGACCAAGTGAGGAGCCTGATGGAGAACAGCG | |
| | ATAGGTGCCAAGACATTAGGAACCTGGCTTTCCTGGGC | |
| | ATCGCCTACAACACCCTGCTGAGGATCGCCGAGATCGC | |
| | TAGGATTAGGGTGAAGGACATCTCTAGGACCGACGGC | |
| | GGAAGGATGCTGATCCACATCGGAAGGACCAAGACCC | |
| | TGGTGAGCACCGCCGGCGTGGAGAAGGCCCTGAGCCT | |
| | GGGCGTGACCAAGCTGGTGGAGAGGTGGATCAGCGTG | |
| | AGCGGCGTGGCCGACGACCCTAACAACTACCTGTTCT | |
| | GTAGGGTGAGGAAAAATGGGGTAGCCGCCCCTAGCGC | |
| | CACATCTCAGCTGAGCACAAGGGCCCTGGAGGGCATC | |
| | TTCGAGGCCACCCATAGGCTGATCTATGGCGCGAAAGA | |
| | CGACAGCGGACAGCGCTACCTGGCCTGGAGCGGTCAC | |
| | AGCGCTAGGGTTGGTGCGGCTAGGGACATGGCTAGGG | |
| | CCGGCGTGAGCATCCCTGAGATCATGCAAGCCGGCGG | |
| | CTGGACCAACGTGAACATCGTGATGAACTACATTAGGA | |
| | ACCTGGACAGTGAGACCGGCGCCATGGTGAGGCTGCT | |
| | GGAGGACGGCGACTGA | |
| Cre opt4 | ATGCCCAAGAAGAAGAGAAAGGTGAGCAACCTGCTG | https://clims4. |
| | ACCGTGCATCAGAACCTGCCCGCCCTGCCCGTGGACG | genewiz.com/ |
| | CCACAAGCGACGAGGTAAGAAAAAACCTCATGGACAT | Toolbox/ |
| | GTTCAGAGACAGACAAGCCTTCAGCGAGCACACCTGG | CodonOptimization |
| | AAGATGCTGCTGAGCGTGTGCAGAAGCTGGGCCGCCT | |
| | GGTGCAAGCTGAACAACAGAAAGTGGTTCCCCGCCGA | |
| | GCCCGAGGACGTGAGAGACTACCTGCTGTACCTGCAA | |
| | GCTAGAGGCCTGGCCGTGAAGACCATTCAGCAGCACC | |
| | TGGGGCAGCTGAACATGCTGCACAGAAGAAGCGGCCT | |
| | GCCTAGACCTAGCGACAGCAACGCCGTGAGCCTGGTG | |
| | ATGAGAAGAATCAGAAAGGAGAACGTGGACGCCGGC | |
| | GAGAGAGCCAAGCAAGCCCTGGCCTTCGAGAGAACC | |
| | GACTTCGACCAAGTGAGAAGCCTGATGGAGAACAGCG | |
| | ACAGATGCCAAGACATAAGAAACCTGGCCTTTCTGGG | |
| | CATCGCCTACAACACCCTGCTGAGAATCGCCGAGATCG | |
| | CTAGAATCAGAGTGAAGGACATCAGCAGAACCGACGG | |
| | CGGCAGAATGCTGATCCACATCGGCAGAACCAAGACC | |
| | CTGGTGAGCACCGCCGGCGTGGAGAAGGCCCTGAGCC | |
| | TGGGCGTGACCAAGCTGGTGGAGAGATGGATCAGCGT | |
| | GAGCGGCGTGGCCGACGACCCCAACAACTACCTGTTC | |
| | TGCAGAGTGAGAAAAAATGGAGTGGCCGCCCCTAGCG | |
| | CCACATCTCAGCTGAGCACAAGAGCCCTGGAGGGCAT | |
| | CTTCGAGGCCACCCACAGACTGATCTACGGCGCCAAG | |
| | GACGACAGCGGGCAGCGGTACCTTGCTTGGAGCGGCC | |
| | ACAGTGCGAGAGTGGGCGCAGCCCGTGACATGGCTAG | |
| | AGCCGGCGTGAGCATCCCCGAGATCATGCAAGCCGGC | |
| | GGCTGGACCAACGTGAACATCGTGATGAACTACATCA | |
| | GAAATCTGGACTCTGAGACCGGCGCCATGGTGAGACT | |
| | GCTGGAGGACGGCGACTGA | |

TABLE S5
Table of plasmid information.

| ID | Plasmid name | Description |
|---|---|---|
| 1 | pAAV-tdTomato (+ITR) | AAV transgene plasmid encoding tdTomato |
| 2 | pRep-Cap-DJ | AAV packaging plasmid: Rep2-Cap-DJ |
| 3 | pAd-Helper | Adeno-helper plasmid |
| 4 | pRAAV-tdTomato-no PS | RAAV-tdTomato transgene plasmid containing no |
| | | packaging signal |
| 5 | pRAAV-tdTomato-1 × MS2 | RAAV-tdTomato transgene plasmid containing 1 × MS2 as |
| | | packaging signal |
| 6 | pRAAV-tdTomato-3 × MS2 | RAAV-tdTomato transgene plasmid containing 3 × MS2 as |
| | | packaging signal |
| 7 | pMRC-DJ | RAAV packaging plasmid: MCP-Rep2-Cap-DJ |
| 8 | pMR^{Y156F}C-DJ | RAAV-v2 packaging plasmid: MCP-Rep2(Y156F)-Cap-DJ |
| 9 | pAAV-Cre (+ITR) | AAV transgene plasmid encoding Cre |
| 10 | pRAAV-Cre-no PS | RAAV-Cre transgene plasmid containing no packaging |
| | | signal |
| 11 | pRAAV-Cre-3 × MS2 | RAAV-Cre transgene plasmid containing 3 × MS2 as |
| | | packaging signal |
| 12 | pPR^{Y156F}C-DJ | RAAV packaging plasmid: PCP-Rep2(Y156F)-Cap-DJ |
| 13 | pCR^{Y156F}C-DJ | RAAV packaging plasmid: COM-Rep2(Y156F)-Cap-DJ |
| 14 | pRAAV-Cre-3 × PP7 | RAAV-Cre transgene plasmid containing 3 × PP7 as |
| | | packaging signal |
| 15 | pRAAV-Cre-3 × com | RAAV-Cre transgene plasmid containing 3 × com as |
| | | packaging signal |
| 16 | pRep-Cap5 | AAV packaging plasmid: Rep2-Cap5 |
| 17 | pRep-Cap8 | AAV packaging plasmid: Rep2-Cap8 |
| 18 | pRep-Cap9 | AAV packaging plasmid: Rep2-Cap9 |
| 19 | pMR^{Y156F}C5 | RAAV-v2 packaging plasmid: MCP-Rep2(Y156F)-Cap5 |
| 20 | pMR^{Y156F}C8 | RAAV-v2 packaging plasmid: MCP-Rep2(Y156F)-Cap8 |
| 21 | pMR^{Y156F}C9 | RAAV-v2 packaging plasmid: MCP-Rep2(Y156F)-Cap9 |
| 22 | pCR^{Y156F}C5 | RAAV-v2 packaging plasmid: COM-Rep2(Y156F)-Cap5 |
| 23 | pCR^{Y156F}C8 | RAAV-v2 packaging plasmid: COM-Rep2(Y156F)-Cap8 |
| 24 | pCR^{Y156F}C9 | RAAV-v2 packaging plasmid: COM-Rep2(Y156F)-Cap9 |
| 25 | pMR^{L454F+D455F}C-DJ | RAAV-v3.1 packaging plasmid: MCP- |
| | | Rep2(L454F + D455F)-Cap-DJ |
| 26 | pMR^{I343T+K447F}C-DJ | RAAV-v3.2 packaging plasmid: MCP- |
| | | Rep2(I343T + K447F)-Cap-DJ |
| 27 | pMR^{A344T+K447F}C-DJ | RAAV-v3.3 packaging plasmid: MCP- |
| | | Rep2(A344T + K447F)-Cap-DJ |
| 28 | pMR^{A344V+K447F}C-DJ | RAAV-v3 packaging plasmid: MCP-Rep2(A344V + K447F)- |
| | | Cap-DJ |
| 29 | pMR^{A344T+L450M}C-DJ | RAAV-v3.5 packaging plasmid: MCP- |
| | | Rep2(A344T + L450M)-Cap-DJ |
| 30 | pMR^{A344V+L450M}C-DJ | RAAV-v3.6 packaging plasmid: MCP- |
| | | Rep2(A344V + L450M)-Cap-DJ |
| 31 | pRAAV-Cre opt1-3 × MS2 | RAAV-Cre opt1 transgene plasmid containing 3 × MS2 as |
| | | packaging signal |
| 32 | pRAAV-Cre opt2-3 × MS2 | RAAV-Cre opt2 transgene plasmid containing 3 × MS2 as |
| | | packaging signal |
| 33 | pRAAV-Cre opt3-3 × MS2 | RAAV-Cre opt3 transgene plasmid containing 3 × MS2 as |
| | | packaging signal |
| 34 | pRAAV-Cre opt4-3 × MS2 | RAAV-Cre opt4 transgene plasmid containing 3 × MS2 as |
| | | packaging signal |
| 35 | pRAAV-Cre opt5-3 × MS2 | RAAV-Cre opt5 transgene plasmid containing 3 × MS2 as |
| | | packaging signal |
| 36 | pRAAV-Cre-stuffer1-3 × MS2 (3857 | RAAV-Cre transgene plasmid containing a 1828 bp stuffer |
| | nt) | sequence |
| 37 | pRAAV-Cre-stuffer2-3 × MS2 (4337 | RAAV-Cre transgene plasmid containing a 2308 bp stuffer |
| | nt) | sequence |
| 38 | pRAAV-optCre-no PS | RAAV- optCre transgene plasmid containing no packaging |
| | | signal |
| 39 | pR^{Y156F}C-DJ | No MCP control of the RAAV-v2 packaging plasmid: MCP- |
| | | Rep2(Y156F)-Cap-DJ |
| 40 | pR^{A344V+K447F}C-DJ | No MCP control of the RAAV-v3 packaging plasmid: MCP- |
| | | Rep2(A344V + K447F)-Cap-DJ |
| 41 | pRep | No Cap control of the AAV packaging plasmid: Rep2-Cap- |
| | | DJ |
| 42 | pMR^{Y156F} | No Cap control of the RAAV-v2 packaging plasmid: MCP- |
| | | Rep2(Y156F)-Cap-DJ |
| 43 | pMR^{A344V+K447F} | No Cap control of the RAAV-v3 packaging plasmid: MCP- |
| | | Rep2(A344V + K447F)-Cap-DJ |
| 44 | pAAV-Cas12Max (+ITR) | AAV transgene plasmid encoding Cas12Max |
| 45 | pRAAV-Cas12Max | RAAV-Cas12Max transgene plasmid containing 3 × MS2 as |
| | | packaging signal |
| 46 | pMR^{A344V+K447F}C9 | RAAV-v3 packaging plasmid: MCP-Rep2(A344V + K447F)- |
| | | Cap9 |
| 47 | pMR^{A344V+K447F}C-PHP.eB | RAAV-v3 packaging plasmid: MCP-Rep2(A344V + K447F)- |
| | | Cap-PHP.eB |

TABLE S6
Primers used in qPCR and RT-qPCR.

| Primer names | Primer sequences (5′-3′) | Description |
|---|---|---|
| CAG-F | CTTCTCCTCCGGGCTGTAAT | for titration of RAAVs |
| CAG-R | CTTTCACGCAGCCACAGAAA | |
| WPRE-F | CCCGTATGGCTTTCATTTTCTCC | |
| WPRE-R | GGCAATGCCCCAACCAGTG | |
| 5′Cre-F | CCAGTAGATGCCACTAGCGA | |
| 5′Cre-R | GCCTGGAGATACAGCAGGTA | |
| 3′Cre-F | CGAAGCTGGTGGAAAGATGG | |
| 3′Cre-R | CAATTGGCTGGTGGCAGAG | |
| Stuffer-F | CCGTGAAGACCATTCAGCAG | |
| Stuffer-R | CACTTGGTCGAAGTCGGTTC | |
| mGAPDH-F | TATGTCGTGGAGTCTACTGGTGTCTT | for detecting the mGAPDH mRNA in |
| | CACC | transduced Ai9-MEFs |
| mGAPDH-R | GTTGTCATATTTCTCGTGGTTCACAC | |
| | CC | |
| 36B4-F | ACTGGTCTAGGACCCGAGAAG | for detecting the 36B4 gene in |
| 36B4-F | TCAATGGTGCCTCTGGAGATT | transduced Ai9-MEFs |
| Cas12Max-F | AGCGGACCAACAAGAGAGAA | for titration of RAAV-Cas12Max |
| Cas12Max-R | ATCATCTTGCAGCCATGCAG | |

TABLE S7
Primers used to amplify on-target and predicted off-trget sites for NGS.

| | | 1st round | | | |
|---|---|---|---|---|---|
| On- and | gRNA | PCR | | NGS | |
| off- | Sequence | primer | 1st round PCR primer | primer | NGS primer |
| targets | (5′-3′) | names | sequence (5′-3′) | names | sequences (5′-3′) |
| hTTR on- | tagaagggat | hTR-ON-1-F | ctccagcctgggtgacaaga | hTR-ON-F | ataggaaaaccagtgagtctg |
| target | atacaaagtg | | g | | gagagc |
| | ga | hTR-ON-1-R | atgtaagttcaagtcatgtta | hTR-ON-R | ccaaaaccaaaacaaccctcg |
| | | | ctaagtggat | | aagg |
| hTTR | TAGAAG | hTR-OT1-1-F | ccattctttgtctgatttgca | hTR-OT1-F | accgtagtcaaacttctctgaa |
| off- | GGATAT | | atgctgatgttac | | tatttgtactagag |
| target | AgAAAG | hTR-OT1-1-R | gttttcactgatatgggagaa | hTR-OT1-R | aacctaacacaatgagaaaca |
| 1 | Tatc | | tggtctgccc | | gaagatattacctac |
| hTTR | TAGCAG | hTR-OT2-1-F | ggtgattggcaaactgttagg | hTR-OT2-F | gtatacatgttcctgtgcctta |
| off- | GGATAT | | aatgaggc | | tactccc |
| target | ACAAAG | hTR-OT2-1-R | gtcatttctcctctttgggag | hTR-OT2-R | acatggcaggaaacaactcct |
| 2 | gGaA | | ttatttcctc | | gct |

### Example 6: Efficient Packaging of RNA into RAAV Viral Particles

This example demonstrates that RNA vector genome can be efficiently packaged into AAV viral capsids, especially with the modified/recombinant RNA designed for direct packaging into AAV capsids.

First, it was surprisingly shown that the AAV packaging signal-ITR (DNA), when transcribed (as RNA), was able to facilitate the packaging of the RNA of the disclosure (e.g., the rRAAV vector genome RNA) into AAV particles, especially when it is presented in certain configurations (e.g., when the transcribed modified AAV ITR sequence is close to the 3′ end of the transcribed RNA sequence of the disclosure).

Specifically, wild-type and modified AAV ITR sequences (DNA) from the ends of the AAV vector genome were moved into their respective transgene expression cassettes, to ensure that all the transgene transcripts (RNA's) contain a candidate packaging signal. In order to block the production of conventional AAV vectors with ssDNA genomes during RAAV production, optimized ITRs (dITR (SEQ ID NO: 2) and dITR-D (SEQ ID NO: 3)) were used instead of wild type ITR (SEQ ID NO: 1).

| ITR Names | Nucleic Acid (DNA) Sequences |
|---|---|
| wild type | TTGGCCACTCCCTCTCTGCGCGCTCGCTCGCTCACTGAGGCCGCCCGGGCAAA |
| ITR2 (Flop) | GCCCGGGCGTCGGGCGACCTTTGGTCGCCCGGCCTCAGTGAGCGAGCGAGCGC |
| SEQ ID NO: 1 | GCAGAGAGGGAGTGGCCAACTCCATCACTAGGGGTTCCT |
| dITR | TCCCTCTCTGCGCGCTCGCTCGCTCACTGAGGCCGCCCGGGCAAAGCCCGGGC |
| SEQ ID NO: 2 | GTCGGGCGACCTTTGGTCGCCCGGCCTCAGTGAGCGAGCGAGCGCGCAGAGA |
| | GGGAGTGG |
| dITR-D | TCCCTCTCTGCGCGCTCGCTCGCTCACTGAGGCCGCCCGGGCAAAGCCCGGGC |
| SEQ ID NO: 3 | GTCGGGCGACCTTTGGTCGCCCGGCCTCAGTGAGCGAGCGAGCGCGCAGAGA |
| | GGGAGTGGACTAGCTCCATCACTAGGGGTTCCT |

Specifically, in the wild-type AAV2 ITR (ITR2) sequence in the Flop configuration, the TRS (“TTGGC”) is at the 5′ end of the ITR, and its reverse complement sequence GCCAA is double underlined. This sequence can be cloned into the coding plasmid in either direction (i.e., either the sequence shown as SEQ ID NO: 1, or its reverse complement sequence, can be used as template to transcribe the RNA of the disclosure). In the experiments herein, the wild-type AAV2 ITR sequence was cloned in an orientation such that the transcribed RNA had the same sequence as SEQ ID NO: t (or SEQ ID NO: 2 or 3 below) except that T's were replaced by U's in the transcribed RNA. Regardless, upon transcription of either this sequence or its reverse transcript, the resulting transcribed RNA of the wild-type ITR2 comprises the palindromic transcribed RBE (shaded in grey). In the experiment herein, the transcribed RNA comprises a transcribed wild-type AAV2 ITR that is equivalent to SEQ ID NO: 1, except that all T's were replaced by U's. If the reverse complement sequence of SEQ ID NO: 1 were used as the DNA template, the transcribed RNA would comprise a transcribed TRS (UUGGC) encoded by GCCAA. The transcribed TRS is located between the transcribed R3E and the transcribed D sequence.

One of the modified ITR sequence is “delta ITR” (or “dITR” for short), which is defective because the dITR lacks both the D region sequence (bold italic), the TRS at the 5′ end, and the reverse complement TRS sequence (“GCCAA”) except for the first G. Upon transcription of this sequence, the transcribed RNA of the dITR also comprises the palindromic transcribed RBE (shaded in grey), and a transcribed defective ITR that lacks a transcribed TRS (UUGGC) encoded by GCCAA. In this experiment, however, the reverse complement sequence of SEQ ID NO: 2 served as the DNA template, such that the transcribed RNA comprises a transcribed modified AAV2 ITR (transcribed dITR) having the same sequence as SEQ ID NO: 2, except that all T's were replaced by U's.

Another one of the modified ITR sequence is “dITR-D,” which is also defective because it retains its D sequence (“CTCCATCACTAGGGGTTCCT,” SEQ ID NO: 4) but lacks the 5′ end TRS (TTGGC). In addition, only the first G in the reverse complement TRS (GCCAA) is retained in dITR-D, and the remaining CCAA sequence is replaced by an unrelated ACTAG sequence. In this experiment, the reverse complement sequence of SEQ ID NO: 3 served as the DNA template, such that the transcribed RNA comprises a transcribed modified AAV2 ITR (transcribed dITR-D) having the same sequence as SEQ ID NO: 3, except that all T's were replaced by U's.

Note that both the dITR and dITR-D sequences retain the shaded palindromic RBE sequence SEQ ID NOs: 42-43, respectively (CTGCGCGCTCGCTCGCTCACTG . . . CAGTGAGCGAGCGAGCGCGCAG), and their respective transcribed modified ITR's also have the RBE sequence.

Such optimized ITR coding sequences (DNA) were inserted into two positions of the tdTomato expression cassette—one located in-between the promoter and the tdTomato coding sequence, and the other located in-between the Woodchuck Hepatitis Virus (WHP) Posttranscriptional Regulatory Element (WPRE) and the SV40 polyA signal.

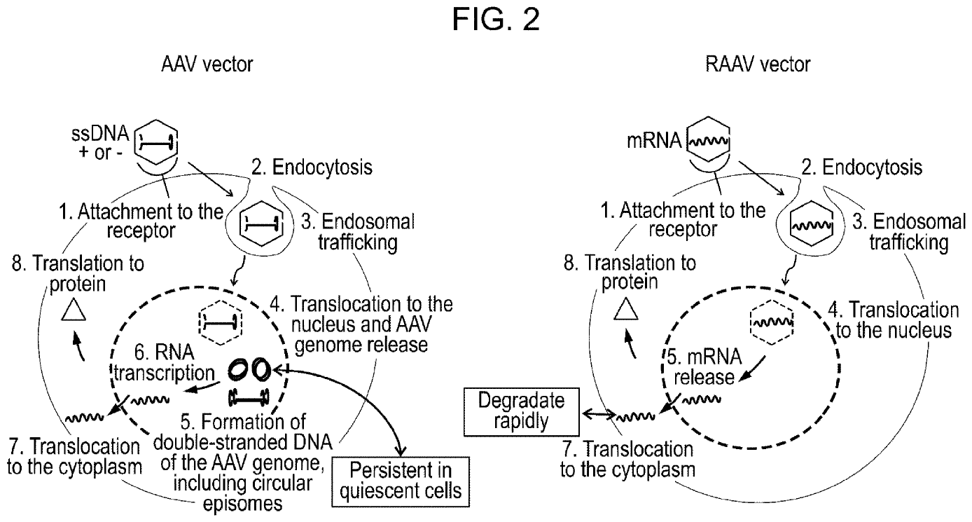

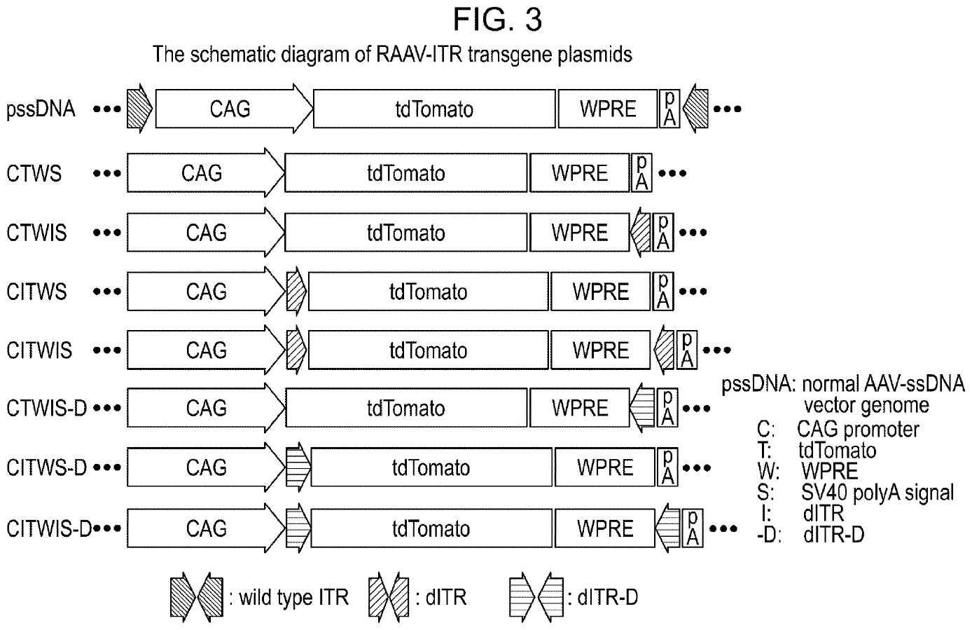

Based on the sequences, numbers and positions of the optimized-ITRs used, a series of the various ITR-based RAAV vectors were constructed (see FIG. 3).

A conventional AAV vector with a ssDNA vector genome and no ITR sequences at either end (“CTWS,” which stands for the sequence elements CAG promoter, tdTomato transgene, WPRE sequence, and SV40 polyA signal sequence) were used as a control. For this experiment, AAV serotype DJ was chosen because of its excellent transduction efficiency in cultured cells used. AAV-DJ is a synthetic serotype with a chimeric capsid of AAV-2, 8, and 9. It contains a heparin-binding domain in its capsid, which may efficiently transduce a broad range of cell types and escape from immune neutralization (Grimm et al., J Virol. 82:5887-5911, 2008).

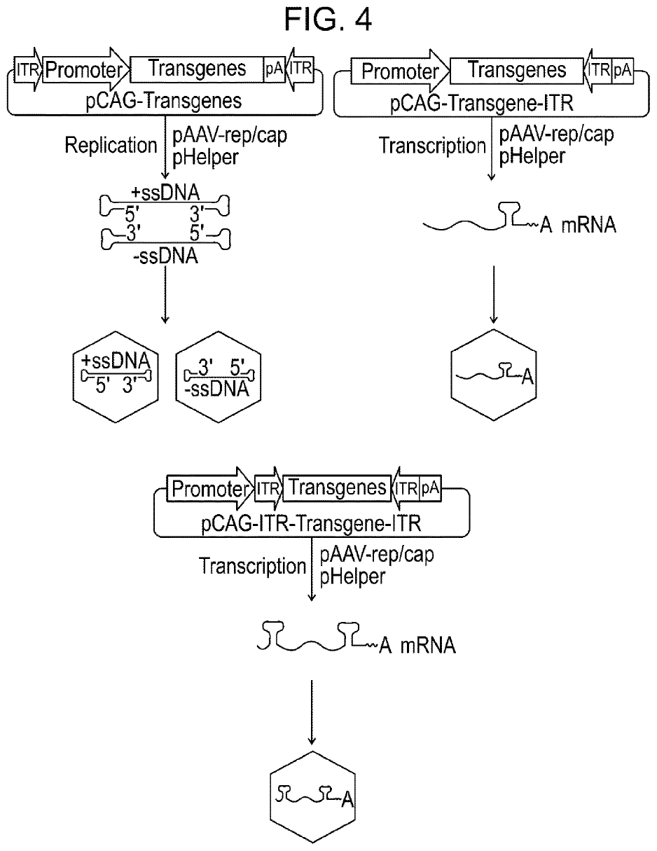

Both the various RAAV-ITR viral particles and the control viral particles were generated by using the triple-plasmid transfection system (FIG. 4).

In particular, the RAAV vectors were generated by co-transfecting transgene plasmid, packaging plasmid and helper plasmid (weight ratio was 1:1:2) into HEK293T cells. The HEK293T cells were cultured in competent DMEM medium, and the cells were plated 24 hrs before transfection. Before transfection, the culture medium was replaced with fresh DMEM containing 2% FBS. PEI-MAX was used as the transfection reagent. The supernatant was then collected at Day 2 and Day 5 post transfection, and transfected cells were harvested on Day 5. RAAV vectors were purified by using iodixanol density gradient ultracentrifugation.

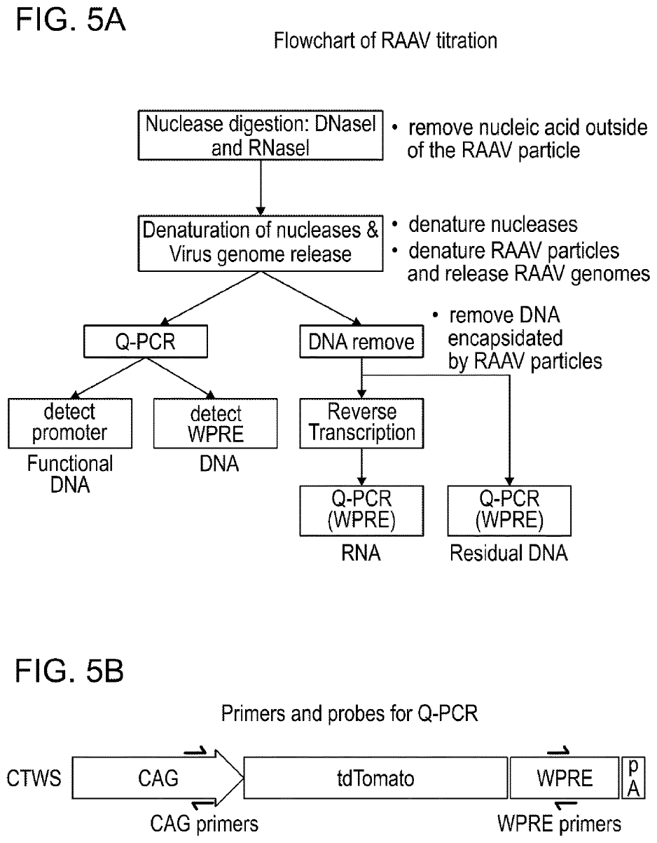

Viral titers (DNA titer and RNA titer) were determined by Q-PCR and Reverse transcription-PCR (RT-PCT), respectively, using the procedure in FIG. 5A.

Briefly, the harvested and purified RAAV viral particles were first subjected to DNase I and RNase I treatment at 37° C. for 2 hours to remove all nucleic acids outside the protein shells of the viral particles. Next, the nucleases were denatured at 100° C. for about 30 min, before the RAAV viral particles were denatured and ruptured to release the RAAV nucleic acid contents for further analysis.

Q-PCR was used to analyze the nuclease-resistant products, in order to titrate the DNA vector genome encapsidated within the RAAV viral particles, Specifically, a primer pair specific for the promoter sequence was used in one set of Q-PCR to detect/quantitate any functional DNA, and a primer pair specific for the WPRE sequence was used in another set of Q-PCR to detect/quantitate any DNA vector genome encapsidated in the RAAV viral particles. See FIG. 5B.

Meanwhile, in another sample, any RAAV-encapsidated DNA was first removed by DNA removal through Dnase I digestion, before the remaining RNA was subjected to reverse-transcription, and the resulting cDNA was used as Q-PCR templates for detection/quantitation of WPRE sequences transcribed into RNA. To detect/quantitate any residue DNA that may be present after incomplete DNA removal, a sample after the DNA removal step was directly amplified using Q-PCR to detect any WPRE (DNA) sequences that might be present in that sample. See FIG. 5A.

To test the packaging efficiency of the CITWS construct (see FIG. 6A), when the conventional pssDNA construct (with wild-type ITR sequences on both ends) was used to generate viral particles, the vast majority of the viral particles contained functional DNA vector genome with promoter sequences and the WPRE sequences. Very occasionally (two orders of magnitude, or about 1% of the time), RNA vector genome was also packaged into viral particles (see the bar labelled as “RNA” which is about 2 orders of magnitude lower than the bar labelled as “DNA” and “Functional DNA”). Residual DNA is one order of magnitude less than the packaged RNA vector genome.

Meanwhile, removing the ITR sequences from both ends of the AAV vector genome essentially abolished packaging—the CTWS construct (ITR-free) in FIG. 6A produced 2-2.5 orders of magnitude less of packaged DNA, and even less RNA.

Adding back only one optimized ITR sequence (either the dITR or dITR-D sequence), between the 3′ of the promoter and 5′ to the GOI coding sequence, did not appear to enhance RNA packaging compared to the CTWS control, though DNA packaging seemed to have slightly improved. See FIG. 6A.

Interestingly, a very different result was achieved in FIG. 6B, in which the CTWIS constructs were tested. Specifically, essentially the same results were obtained regarding packaging the pssDNA constructs (compared FIGS. 6A and 6B)—most packaged viral particles contained DNA (99% or more) and negligible amount (1% or less) of RNA. However, including an optimized ITR sequence (dITR) between the 3′ end of the WPRE sequence and the 5′ end of the polyA sequence significantly reduced or even reversed the packaging efficiency difference between DNA and RNA. This effect is even more prominent when the dITR-D sequence was used, when the vast majority of the packaged nucleic acids are RNA (1-2 orders of magnitude over packaged DNA).

Essentially the same results were obtained if one additional (i.e., a second) optimized ITR sequence was inserted between the promoter and the GOI coding sequence in the CITWIS constructs. See FIG. 6C.

These results demonstrated that, optimized ITRs (dITR and dITR-D) impaired the replication of the conventional AAV vectors, thereby leading to a reduction of DNA packaging into the RAAV viral particle.

Compared to the control vector CTWS (ITR-free) and the RAAV-dITR vectors, RAAV vectors with the dITR-D optimized ITR seem to have a better ability to encapsidate the transcribed mRNAs directly into RAAV particles, especially when the dITR-D ITR is located downstream of the mRNA coding sequence and WPRE sequence (for example, just 5′ to the polyA signal). See FIG. 6A-6C. In contrast, a dITR-D sequence located upstream of the mRNA coding sequence (e.g., right after the promoter sequence in the expression construct) hardly facilitated direct mRNA packaging into the RAAV viral particles. Meanwhile, if another dITR-D sequence was inserted downstream of the mRNA coding sequence (e.g., right 5′ to the polyA sequence), packaging of the resulting RAAV mRNAs was similarly highly increased (FIG. 6C).

In conclusion, CITWIS-D, which harbours dITR-D signals at both ends of its mRNA genome, has the best ability to encapsidate specific mRNAs, despite the fact that its yield (mRNA-harbouring particles) is 20-fold lower than the yield of conventional AAV vectors with ssDNA vector genomes (pssAAV group). Unlike the conventional AAV vectors, RAAV vector CITWIS-D have an impaired DNA packaging, with its DNA-carrying particles only taking up about 20% or less of the RAAV vector stock, and the percentage of the particles harbouring functional DNAs is even lower (e.g., less than 10%) (FIG. 6A-6C).

Since the AAV packaging capacity is limited (<4700 nt), the undesired RAAV DNA packaging could be reduced by enlarging the size of the transgene plasmids, and functional DNA packaging could be further reduced by increasing the length of the transgene cassette, for example, by inserting cis-acting elements (such as, enhancer, intron, etc.) or non-functional stuffer sequence into the cassette.

### Example 7: The RAAV Viral Particles are Functional

This example demonstrates that the subject RAAV-dITR-D vectors are infectious and can be used as gene delivery vectors.

The same volume of purified RAAV-dITR-D (CITWIS-D) vectors were used to infect 2×10⁵ HEK293T cells in vitro, at the same MOI of about 50,000 (the MOI of the CITWIS-D vectors was calculated based on the sum of the number of DNA-particles and mRNA-particles).

Specifically, HEK293T cells were plated into 24-well plates about 24 hrs before infection. RAAV vectors were then mixed completely with 1 mL of DMEM (containing 2% FBS). The culture medium of the cells was then removed, and the cells were incubated with mixed RAAV vectors overnight. Fluorescence photos were taken 3 and 5 days post infection.

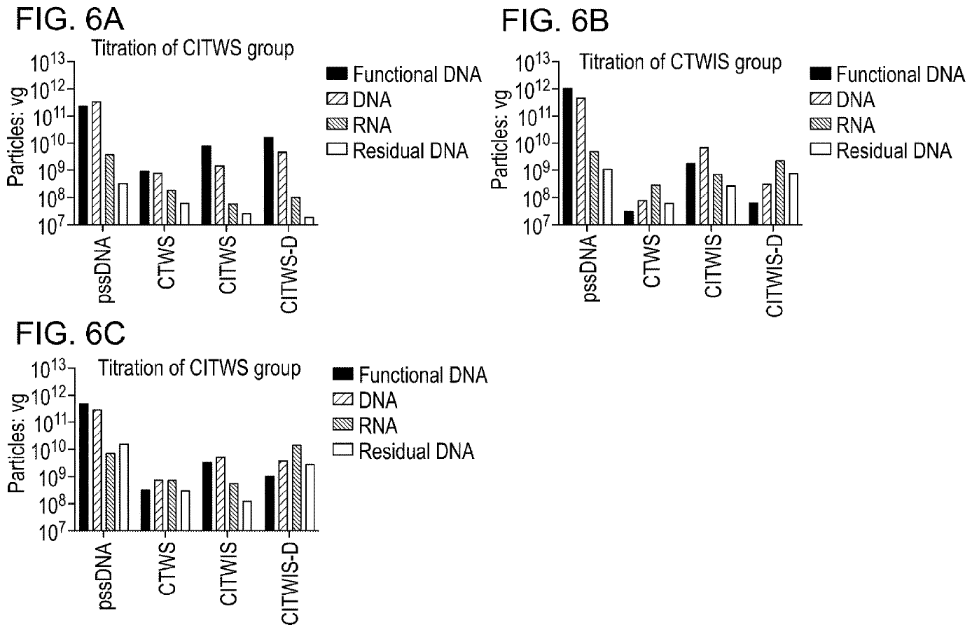

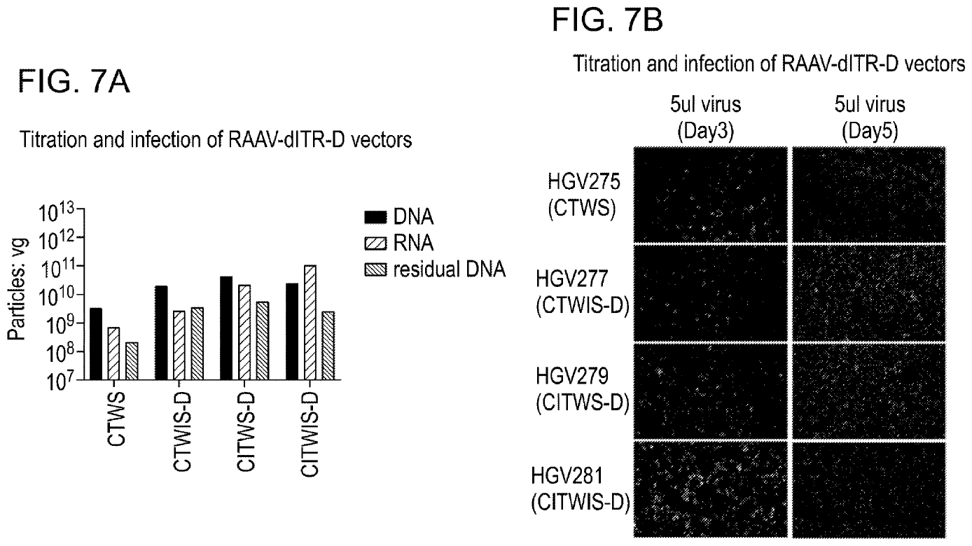

The results showed that, tdTomato expression by the CITWIS-D vector was quicker than that of the other vectors, but the expression was down-regulated rapidly too (see FIG. 7B). This quick expression and degradation phenomenon may be due to the short lifetime of its mRNA genome (FIG. 7B).

It is interesting to note that the CTWS construct without any ITR sequences were apparently packaged to some degree, though the precise mechanism underlying this packaging remains unclear. At least two possibilities can explain the packaging of mRNA vector genome when the CTWS vectors were used: overexpressed cellular mRNAs could be packaged into the RAAV vectors non-specifically, or CTWS mRNA might have some RNA structures that interact with Rep2 or Cap-DJ. Meanwhile, CTWS DNA packaging may be due to the small size of the plasmid CTWS, and DNA packaging may be reduced by increasing the size of the CTWS plasmid.

### Example 8: Efficient Packaging of RNAs into RAAV Particles

This Example demonstrated that RNA genomes can be efficiently packaged into AAV capsids, especially with the modified/recombinant RNA constructs designed herein for direct packaging into AAV capsids to produce RAAV particles.

### 1. Design

The inventors have designed a strategy to utilize the strong interaction between bacteriophage-derived MS2 coat protein (MCP) and its recognizing stem loop MS2 as a novel packaging signal for packaging heterologous RNA into DNA virus viral particles.

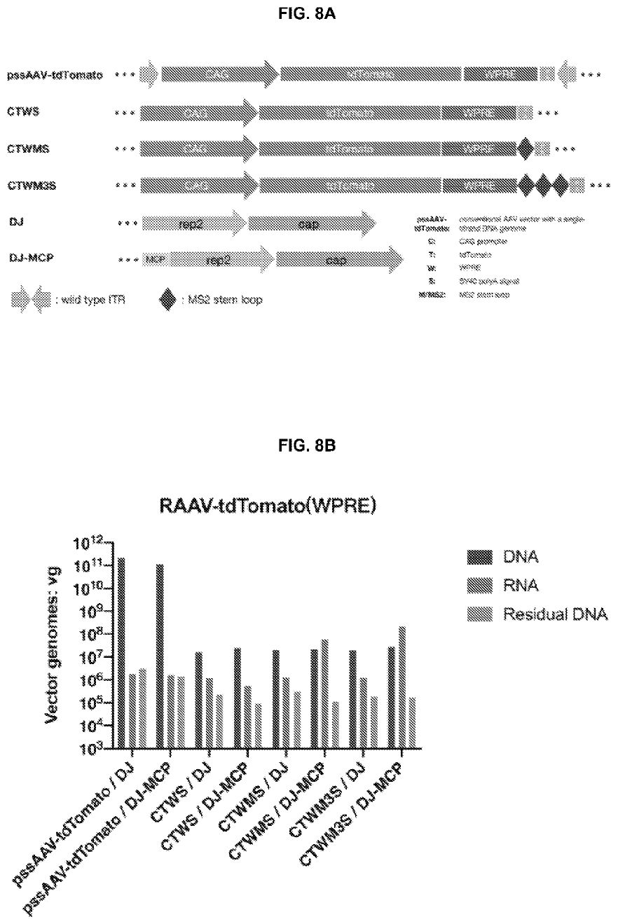

First, in order to inhibit/reduce the production of conventional AAV particles with packaged ssDNA genomes during RAAV production, the conventional AAV packaging signals, ITRs, were removed. Instead, one copy or three copies of RNA packaging signals (RPS), MS2 stem loop (or “MS2” for short; See Table B for its sequence), were inserted into a tdTomato expression cassette, in-between the Woodchuck Hepatitis Virus (WHP) Posttranscriptional Regulatory Element (WPRE) and SV40 polyA signal, in order to ensure that all the transcribed mRNAs would have the RPS, so as to be recognized by the binding protein, bacteriophage-derived MS2 coat protein (or “MCP” for short; See Table A for its sequence), corresponding to the MS2 (FIG. 8A).

Since AAV Rep proteins are non-structural proteins, and they conventionally serve as bridges between the ITRs of ssDNA genomes and the AAV capsids during AAV packaging, MCP was fused to the N-terminus of Rep78 protein and Rep68 protein from AAV2 (Rep68 is a C-terminal truncation of Rep78; See Table C for their sequences). The ability of these MCP-Rep78/68 fusions to interact with the MS2 sequence harbored inside the RNA genomes and to facilitate the packaging of the RNA genomes into the AAV capsids was verified.

The conventional AAV vector pssAAV-tdTomato (with two wild type functional ITRs) and CTWS without functional wild-type ITRs (“CTWS,” which stands for the sequence elements CAG promoter, tdTomato transgene, WPRE sequence, and SV40 polyA signal sequence) were used as controls (FIG. 8A). In this Example, AAV serotype DJ (“AAV-DJ” or “DJ”) was selected for use because of its excellent transduction efficiency in the cultured cells, HEK293T cells, used in this Example. AAV-DJ is a synthetic serotype with a chimeric capsid of AAV-2, 8, and 9.

### 2. RAAV Packaging and Production

Both the RAAV and control AAV particles herein were produced by using conventional triple-plasmid transfection system mutatis mutandis, by co-transfecting the respective transgene plasmids, packaging plasmids, and helper plasmids in a weight ratio of 1:1:2 into HEK293T cells.

Specifically, the HEK293T cells were cultured in competent DMEM medium, and the cells were plated 24 hrs before transfection. Shortly before transfection, the culture medium was replaced with fresh DMEM containing 2% FBS. PET-MAX was used as the transfection reagent. Transcription of the RPS-harbouring transgene plasmids to generate the RNA genomes to be packaged occurred after the transfection into the infected cells. The supernatant was then collected at Day 2 and Day 5 post transfection, and the transfected cells were harvested on Day 5. The RAAV and control AAV particles were purified by using iodixanol density gradient ultracentrifugation.

### 3. Detection of Packaged Genomes

The purified RAAV and control AAV particles were first subjected to nuclease treatment, including DNase I and RNase I treatment, at 37° C. for 2 hours, in order to remove possibly existed nucleic acids outside the viral particles. Next, the nucleases and the RAAV or control AAV particles were denatured by proteinase K/SDS digestion at 65° C. for about 3 hrs to rupture the viral particles in order to release the genomes packaged therein. The nuclease-resistant polynucleotides containing the released viral genomes were then extracted and purified by phenol/chloroform extraction.

To detect the DNA genome titer of the control AAV and RAAV particles, Q-PCR was used to analyze the nuclease-resistant polynucleotides directly. A pair of WPRE primers (as set forth in the sequence tables below) specific for the WPRE sequence on the viral genomes was used in the Q-PCR to detect and quantitate any DNA genomes encapsidated in the control AAV or RAAV particles.

To detect the RNA genome titer of the control AAV and RAAV particles, any control AAV- or RAAV-encapsidated DNA genomes was removed by DNA removal through Dnase I digestion, before the encapsidated RNA genomes were subjected to reverse-transcription, and the resulting cDNA was used as Q-PCR templates for the detection and quantitation of WPRE sequences with the same pair of WPRE primers aforementioned. To detect and quantitate any potentially residual DNA that might be present due to the incomplete DNA removal, a sample after the DNA removal step was directly amplified (without reverse-transcription) using Q-PCR to detect the WPRE (DNA) sequence with the same pair of WPRE primers aforementioned, which was also used for all the other PCR reactions specific for WPRE sequence.

### 4. Comparison of Packaging Efficiency

When the conventional transgene plasmid containing the pssAAV-tdTomato construct (with wild-type ITRs on both ends) and the conventional packaging plasmid for AAV-DJ were used to produce the control AAV particles, the vast majority of the particles contained DNA genomes with the WPRE sequences. Very occasionally, RNA genomes were also packaged into the particles (see the bar labelled as “RNA,” which was about 5 orders of magnitude lower than the bar labelled as “DNA”). The presence of residual DNA is comparable to that of RNA, which may be due to the inefficient digestion of packaged DNA genomes with DNase I before reverse-transcription.

Interestingly, when the recombinant packaging plasmid DJ-MCP (MCP fused to the N-terminus of Rep78 and 68 proteins) was used instead of DJ, the packaging of DNA genomes was slightly reduced (about 0.5 order of magnitude lower), but the pattern of viral genome distribution was almost the same, which DNA packaging was about 5 orders of magnitude higher than RNA packaging. This result indicated that fusing MCP to the N-terminus of Rep78/68 proteins did not significantly impair their natural functions (FIG. 8B).

Meanwhile, removing the ITRs from both ends of the pssAAV-tdTomato construct, leading to the CTWS construct, significantly abolished DNA packaging. The CTWS construct (ITR-free) produced about 4 orders of magnitude less of packaged DNA, and even less packaged RNA, no matter which packaging plasmid (DJ or DJ-MCP) was used.

Further, by adding one or three copies of the RPS (MS2) between the 3′ of the WPRE and 5′ of the SV40 polyA signal on the viral genomes without ITRs, the RAAV transgene plasmids, CTWMS and CTWM3S respectively, were obtained. In the absence of MCP, the CTWMS and CTWM3S constructs could barely be encapsidated as DNA or RNA genomes, just like the genome distribution pattern of CTWS. Surprisingly, the use of DJ-MCP as the packaging plasmid instead of DJ significantly reversed the packaging efficiency difference between DNA and RNA genomes, and the vast majority of the packaged genomes were RNA.

Compared to CTWS/DJ-MCP, the numbers of the packaged RNA genomes of CTWMS/DJ-MCP and CTWM3S/DJ-MCP were about 100- and 400-fold higher, respectively, whereas no significant difference was observed in the DNA-packaged number of the three. This result suggested that the MCP-Rep78/68 fusions could recognize the RPS, MS2, embedded in the RNA transcripts of the CTWMS and CTWM3S plasmids specifically and facilitate their RNA packaging into RAAV particles, and three copies of RPS in the CTWM3S construct provided an even better RNA packaging efficiency than one copy (FIG. 8B).

In conclusion, the introduction of the MS2/MCP pair into conventional AAV packaging system enabled the packaging of MS2-harboring RNA genomes into AAV particles in the presence of the MCP-Rep78/68 fusions, leading to the generation of RAAV particles. The undesired DNA packaging only constituted about 10% of the whole viral particle population produced by using CTWM3S/DJ-MCP.

In other words, the artificial/heterologous RNA packaging signal (RPS)—the MS2 sequence—can be used with its cognate binding protein MCP to replace the native DNA virus packaging signal pair (i.e., ITR and Rep), in order to dramatically boost the packaging efficiency of RNA into an otherwise DNA virus, while suppressing its inherent packaging of DNA into the same DNA virus.

### Example 9: Enlarged Plasmid Backbone Reduced Undesired DNA Packaging of RAAV

This example demonstrates that increasing the backbone size of the AAV transgene plasmid by inserting a stuffer sequence into the backbone of the plasmid could reduce undesired DNA packaging into RAAV particles.

Although the CTWMS and CTWM3S constructs for RAAV particles in Example 3 did not have ITRs and no reverse packaging existed in the RAAV production, it was speculated that the relative small size (5~6 kb) of the RAAV transgene plasmids might still lead to undesired DNA packaging.

Therefore, a 3266 bp non-coding sequence (stuffer sequence; see the sequence tables below) was inserted upstream of the tdTomato expression cassette of CTWM3S in order to increase the backbone length of the CTWM3S transgene plasmid, and the resulting construct was named L-CTWM3S. The schematic diagram of the plasmid is shown in FIG. 9A.

The conventional AAV genome construct, pssAAV-tdTomato, and the RAAV genome construct, CTWM3S, used in Example 3 were used as controls herein. In the same way as in Example 3, RAAV particles were produced by co-transfecting CTWM3S or L-CTWM3S transgene plasmid together with the packaging plasmid DJ-MCP and the helper plasmid into HEK293T cells, and the resulting RAAV particles were purified and the viral genomes were quantified. The same pair of WPRE primers were used to detect and quantitate any DNA and RNA genomes encapsidated in the AAV and RAAV particles, and an additional pair of CAG primers specific for the CAG promoter sequence in the viral genomes were used in Q-PCR to detect and quantitate any functional DNA (meaning DNA containing the CAG promoter sequence and able to express functional transgene proteins).

It was noted that the packaged RNA genomes cannot be detected with the CAG primers since they did not contain the CAG promoter, and the RNA columns on the drawings with CAG primers represented background RNA signals (see FIG. 9B).

Surprisingly, the DNA genome titer of the L-CTWM3S group was about 2 times lower than that of the CTWM3S group, no matter which pair of primers was used in Q-PCR (see FIGS. 9B and 9C). The RNA genome titers of the CTWM3S and L-CTWM3S groups were substantially equivalent (see FIG. 9C). Since there is no CAG promoter sequence in the transcribed RNA from the CTWM3S and L-CTWM3S transgene plasmids, the packaged RNA genomes could only be detected with the pair of WPRE primers (FIG. 9C).

In conclusion, increasing the backbone length of the transgene plasmid could reduce undesired DNA packaging of the RAAV particles without interfering with their RNA packaging, showing that the deconstruction of the DNA packaging system and the establishment of the RNA packaging system in AAV particles are two separate lines, and this long-stuffer sequence was used to the RAAV transgene plasmids in the subsequent Examples.

### Example 10: Using RAAV-MS2/MCP System for Additional Transgenes

In order to verify the general applicability of the RAAV-MS2/MCP system to additional transgenes, and to ensure that the observed RNA packaging is not a mere artifact associated with the reporter gene used, a series of AAV and RAAV transgene plasmids containing a Cre recombinase expression cassette were generated.

Conventional pssAAV-Cre (with the tdTomato coding sequence in the pssAAV-tdTomato construct in FIG. 8A replaced with a Cre coding sequence) and a corresponding L-CCWS construct (with the tdTomato coding sequence in CTWS in FIG. 8A replaced with the Cre coding sequence and inserted with the stuffer sequence in Example 4) were used as controls, with the second C standing for the Cre recombinase transgene. The L-CTWM3S construct in Example 4 was also redesigned as L-CCWM3S construct, after replacing the tdTomato coding sequence with the Cre coding sequence.

The Cre transgene plasmids were co-transfected with the packing plasmid DJ or DJ-MCP, and together with the helper plasmid in HEK293T cells, respectively, to produce AAV and RAAV particles. The resulting viral particles were purified, and the viral genomes were quantified as described in Example 3.

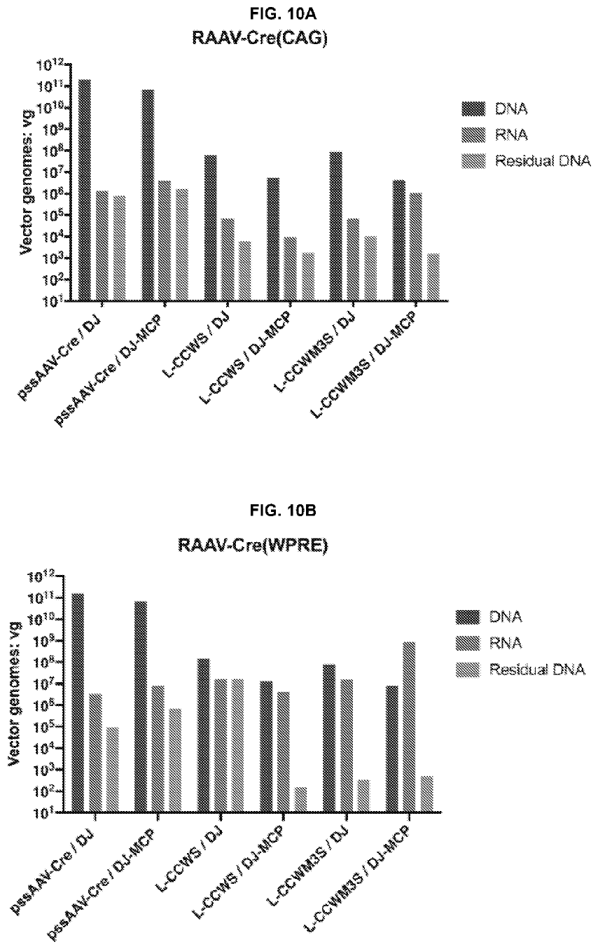

The same viral genome distribution results as AAV-tdTomato and RAAV-tdTomato were achieved for AAV-Cre and RAAV-Cre. For pssAAV-Cre, most viral particles contained DNA genomes, and the DNA genome titer was about 4-5 orders of magnitude higher than that of RNA genomes. For L-CCWS, DNA and RNA genomes were barely encapsidated, due to the lack of both DNA and RNA packaging signals. For L-CCWM3S, RNA packaging was significantly improved with DJ-MCP by about 200-fold compared to that of L-CCWS/DJ-MCP, and the undesired DNA-harbouring viral particles only constituted about 1% of the whole viral particle population (FIGS. 10A and 10B).

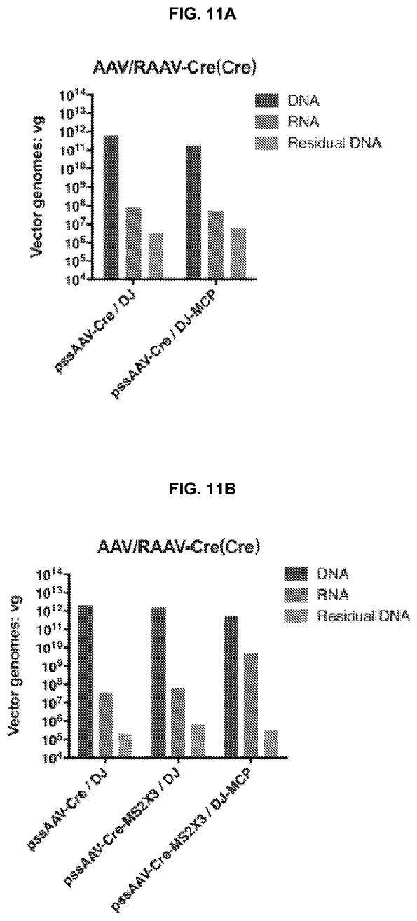

Since the DJ-MCP fusion not only assisted the RNA packaging but also retained the DNA packaging ability, its performance was also assessed in a construct containing both DNA packaging signals (ITRs) and RNA packaging signals (3 copies of MS2) designated as pssAAV-Cre-MS2X3, which was constructed by inserting 3 copies of MS2 in-between WPRE and SV40 polyA of the pssAAV-Cre construct. The results showed that in the absence of MCP, most viral particles contained packaged DNA genomes, and only a negligible amount of RNA genomes was packaged with or without the RNA packaging signals. The RNA packaging was remarkably improved when DJ-MCP was used instead of DJ as the packaging plasmid in combination with the RNA binding signals, and surprisingly, the increased RNA packaging did not significantly interfere with the DNA packaging of the pssAAV-Cre-MS2X3 construct (FIG. 11B).

In another view, it was also demonstrated that the introduction of the MS2/MCP pair could significantly increase RNA packaging even without removing the DNA packaging signal-ITRs, indicating that the deconstruction of the DNA packaging system and the establishment of the RNA packaging system in AAV particles are two separate lines and the removal of ITRs is not the essential basis for the increased RNA packaging by the introduction of RPS/RBP pair.

In conclusion, the subject RAAV-MS2/MCP system can be applied to any transgenes in general, such as the Cre recombinase as demonstrated above. Interestingly, the RAAV-Cre construct produced a better yield than that of the RAAV-tdTomato construct. While not wishing to be bound by any particular theory, this may be due to the simpler secondary structure of the Cre mRNA comparing to the tdTomato mRNA, based on online RNA secondary structure prediction such as that found at ma.tbi.univie.ac.at/cgi-bin/RNAWebSuite/RNAfold.cgi.

### Example 11: Optimization of RAAV Production System and Identification of the Properties of Optimized RAAV Particles

The endonuclease activity of the Rep68 and Rep78 proteins (Rep68/78) is essential for the DNA genome replication during the conventional DNA packaging of AAV particles. Without the functional trs-endonuclease, the newly-synthesized viral ssDNA cannot be released for packaging. It was investigated in this Example whether the undesired DNA packaging of RAAV particles could be further reduced by disrupting the activity of the trs-endonuclease.

To investigate this, three trs-endonuclease negative mutants were constructed, namely DJ-MCP (Y156F, wherein the Y156F mutation was in the common sequence of Rep68 and Rep78 proteins, i.e., Rep68-Y156F and Rep78-Y156F), DJ-MCP (KDE-mu) and DJ-MCP (EKE-mu) (see the sequence tables below).

The DNA and RNA packaging efficiencies for DJ-MCP (Y156F) were firstly assessed with the transgene plasmid, pssAAV-Cre-MS2X3 containing both the DNA and RNA packaging signals, as described in Example 5. DJ and DJ-MCP were set as packaging plasmid controls. Viral particles were produced, purified, and titrated as described in Example 3.

The results demonstrated that the Y156F mutation in DJ-MCP significantly reduced the ITR-mediated DNA packaging for pssAAV-Cre-MS2X3 without interfering with the RNA packaging.

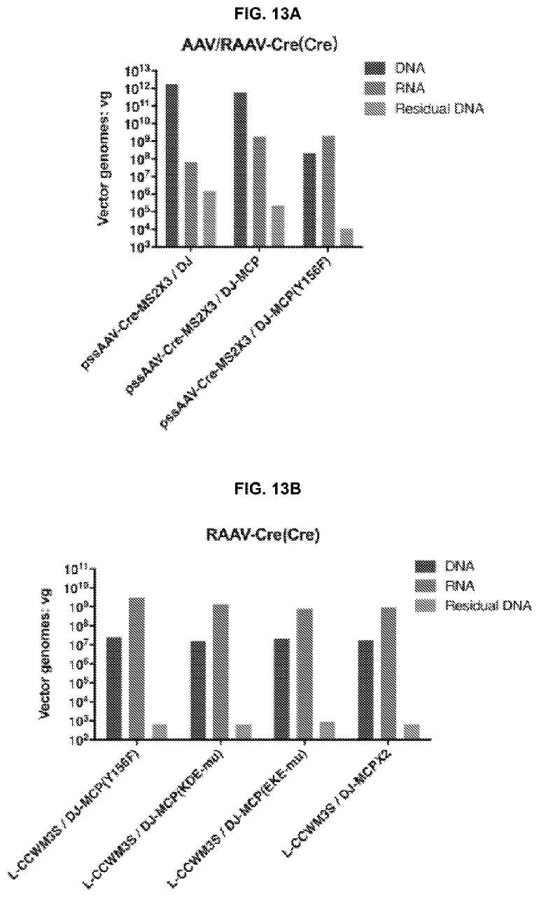

Therefore, in addition to the removal of DNA packaging signals ITR as shown in the previous Examples, modifying, for example, mutating the functional proteins like Rep78/68 proteins participating in the DNA packaging process to weaken or eliminate their DNA-packaging-associated functions could serve as another strategy to reduce or inhibit the conventional DNA packaging of AAV particles (FIG. 13A).

Then, L-CCWM3S in Example 5 was used as a RAAV transgene plasmid to provide viral genomes in place of pssAAV-Cre-MS2X3. The DJ-MCP, which was trs-endonuclease positive, was used as a control against DJ-MCP (Y156F). Viral particles were produced, purified, and titrated as described in Example 3. Two pairs of primers were used here to titrate viral genomes, one pair for targeting WPRE sequence as above and one pair (see sequence tables below) for targeting the 5′ terminus of the Cre coding sequence.

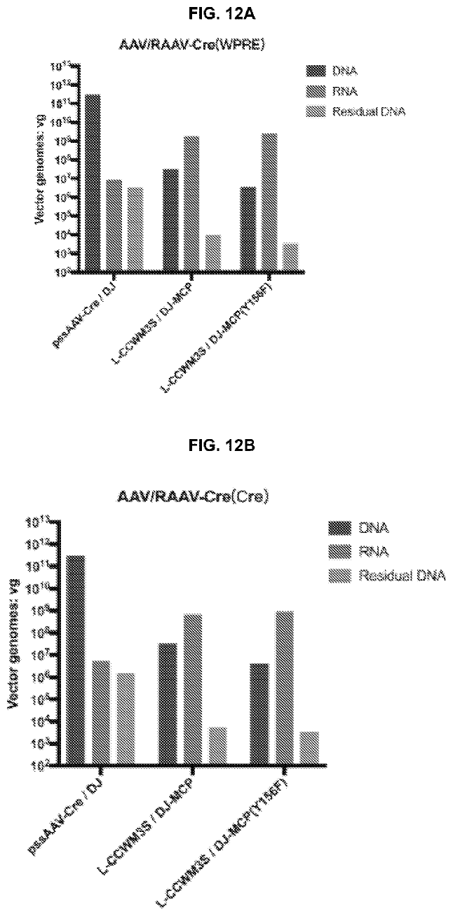

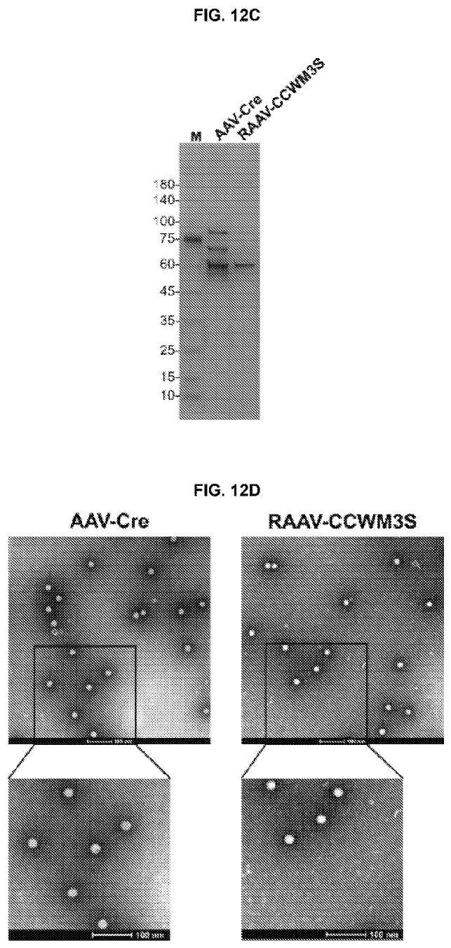

The results showed that the Y156F mutation in Rep78/68 protein not only reduced the undesired DNA packaging by about 10-fold, but also increased the desired RNA packaging by about 50%. The patterns of the packaging efficiency difference between the packaged DNA and RNA genomes were substantially the same for both pairs of primers (i.e., WPRE primer pairs and Cre primer pairs) used in Q-PCR (FIG. 12A-12B).

Two other trs-endonuclease mutants, DJ-MCP (KDE-mu) and DJ-MCP (EKE-mu), were also tested, and were demonstrated to have the same ability to reduce undesired DNA packaging as DJ-MCP(Y156F), but only DJ-MCP (Y156F) showed improved RNA packaging (FIG. 13B).

It was further demonstrated that fusing two copies of MCP to the N-terminus of Rep 78/68 proteins (MCPx2-Rep78 and MCPx2-Rep68) could also achieve the result of reducing undesired DNA packaging (FIG. 13B).

The compositions of the AAV and RAAV particles were analyzed by silver-stained SDS-PAGE, and the RAAV capsids were also composed of three VP proteins (VP1, VP2 and VP3) with a similar VP1/2/3 ratio to conventional AAV particles (FIG. 12C).

In order to analyze the morphology of the RAAV particles, 10 μL of the purified AAV and RAAV particles were placed on a 300 μm carbon-over-Pioloform-coated copper grid and incubated for 2 min. at room temperature. The excess of the sample was blotted with filter paper and immediately replaced by 10 μL of staining agent (3% phosphotungstic acid), which was allowed to settle for 2 min. and then blotted again. Visualization of the samples was performed by using a Talos L120C transmission electron microscope. The RAAV particles were morphologically similar to the conventional AAV vectors, where full viral particles encapsidating genomes were viewed as 25-nm solid spheres, and empty viral particles without genomes encapsidated were 25-nm donut-like structures (FIG. 12D).

In conclusion, the mutation of functional proteins including Rep proteins participating in the DNA packaging process of AAV production to weaken or eliminate their DNA-packaging-associated functions in combination with the removal of DNA packaging signals, ITRs, is an optimized strategy to reduce or inhibit undesired DNA packaging of RAAV particles. The produced RAAV particles have similar compositions and morphology to the conventional AAV particles.

### Example 12: RAAV Vectors Expressing Functional Proteins

This Example demonstrated that the subject RAAV vectors were infectious and can be used as gene delivery vectors.

Cre-loxP system, a highly sensitive system, was used for investigating the infectivity of the inventive RAAV vectors. Mouse embryonic fibroblast (MEF) cells isolated from homo-Ai9 (bearing loxP-tdTomato-reporter system) mice were incubated with the purified AAV (pssAAV-Cre/DJ) or RAAV (L-CCWM3S/DJ-MCP (Y156F)) vectors in Example 5 overnight, and Multiplicity of Infections (MOIs) (the number of virions added per cell during infection) were set, including 7 MOIs for conventional AAV vectors and 3 MOIs for RAAV vectors. The dominant genome titer quantified by detecting Cre coding sequence with the 5′-terminus Cre primers aforementioned was used as the infection titer. In other words, the DNA genome titer was used for the conventional AAV vectors, and the RNA genome titer was used for the RAAV vectors.

Specifically, Ai9-MEF cells were plated into 48-well plates in about 5×10⁴ cells per well about 24 hrs before infection. AAV vectors or RAAV vectors were mixed completely with 0.5 mL of DMEM containing 2% FBS. The culture medium of the plated cells was removed, and then the cells were incubated with mixed AAV or RAAV vectors overnight at 37° C. The infected cells were collected at different time points and subjected to RNA and DNA analysis. A pair of primers targeting the 5′-terminus of Cre-coding sequence as aforementioned was used for detecting the specific Cre-coding DNA and mRNA derived from the vectors. Fluorescence photos were taken daily post infection (p.i), and the fluorescence-positive cells were quantified by flow cytometry 5 days p.i.

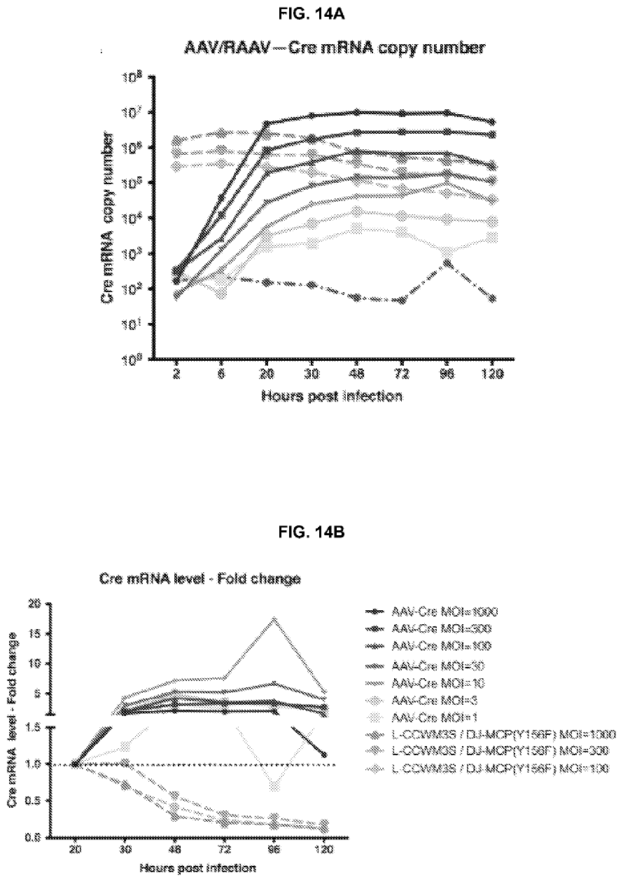

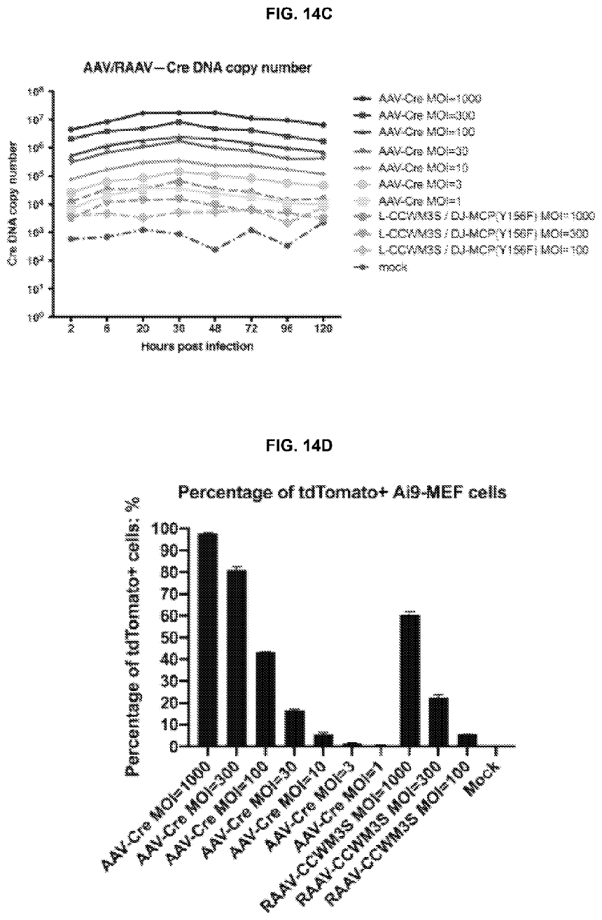

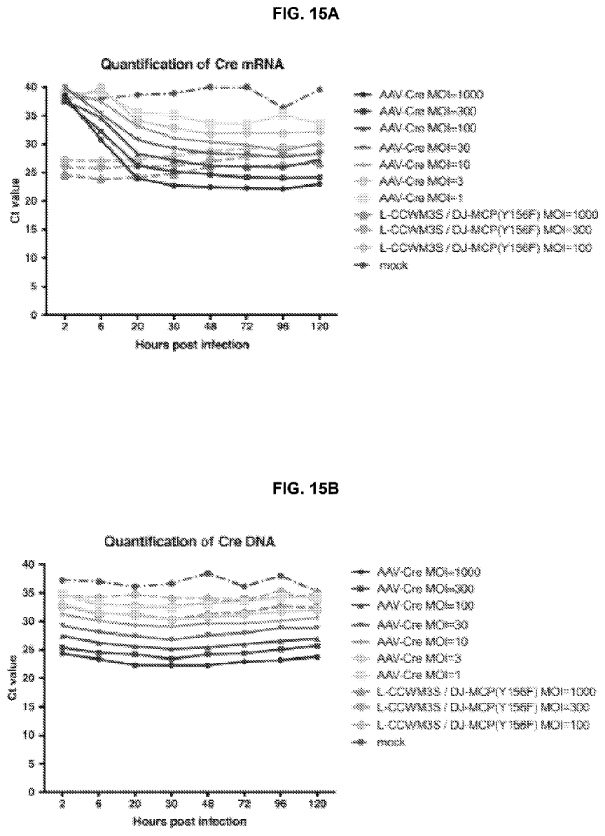

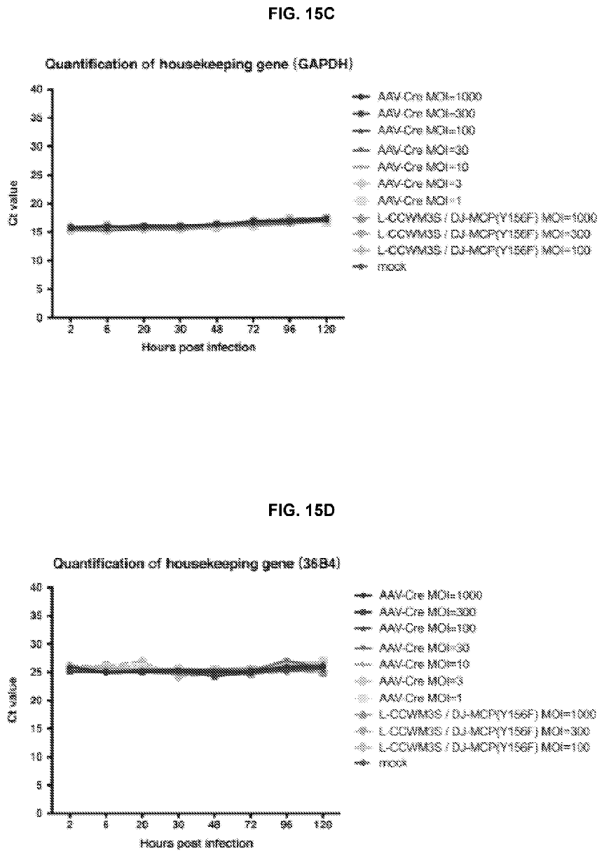

The mRNA analysis results showed that the specific mRNA was detected in the RAAV-infected cells as early as 2 hrs p.i, peaked at 6 hrs p.i, and then decreased. In the cells infected with the conventional AAV vectors, no apparent transcription was detected at 2 hrs p.i, but a rapid increase of transcribed mRNA was observed from 6 hrs to 20 hrs p.i, reaching a plateau at 30 hrs. In contrast to the results for the RAAV vectors, the mRNA level in the cells infected with the conventional AAV vectors did not decrease after reaching the plateau. The copy numbers of the Cre mRNAs were positively correlated with MOIs in all the samples (FIG. 14A-14B and FIG. 15A). Meanwhile, mGAPDH mRNA was tested as a reference transcript (housekeeping gene), and as expected, there was no difference in mGAPDH mRNA levels among all the samples (FIG. 15C).

The DNA results were quite different from the mRNA results. Conventional AAV and RAAV vectors had substantially the same DNA copy number pattern, the majority of DNA genomes was detected in the infected cells as early as 2 hrs post infection, and then a slight increase followed from 2 hrs to 20 hrs p.i, which was very similar to the trend of the mRNA levels in the RAAV-infected cells. After that, the DNA level reached a plateau or descended slowly. The copy numbers of the Cre DNA were also positively correlated with MOIs in all the samples, but much lower numbers of the Cre DNA were detected in the RAAV-infected cells (the DNA copy number of RAAV-CCWM3S MOI=100 or 300 group was less than that of AAV-Cre MOI=1 group, and the DNA copy number of RAAV-CCWM3S MOI=1000 group was less than that of AAV-Cre MOI=3 group) (FIG. 14C and FIG. 15B). Similarly, the DNA level of another housekeeping gene 36B4 was quantified as a reference gene, and as expected, no obvious difference in the DNA levels was observed among all the samples (FIG. 15D).

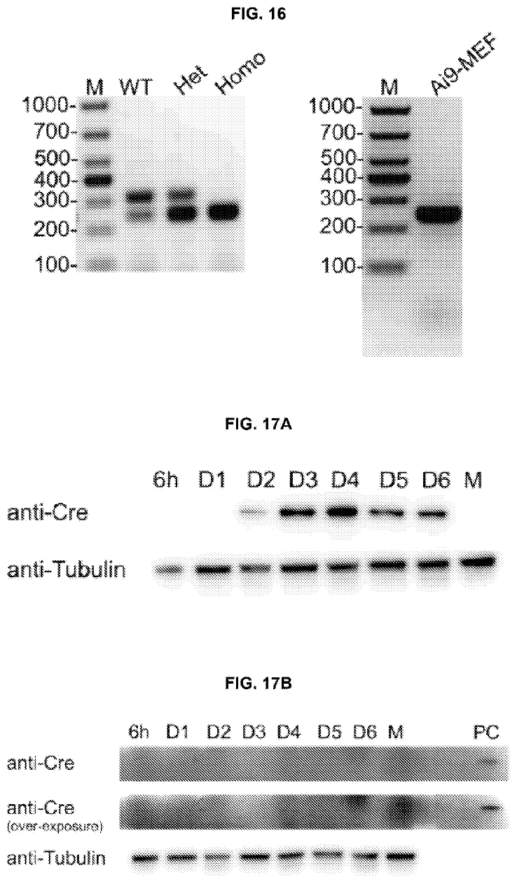

Successful infection of AAV-Cre or RAAV-CCWM3S vectors would lead to the expression of functional Cre recombinase and rescue the tdTomato expression in Ai9-MEF cells, and thus the fluorescence photos of the infected cells were taken and analyzed to assess the infectivity of the viral vectors by counting cells emitting tdTomato red fluorescence. The results showed that the number of fluorescence-positive cells generated by RAAV-CCWM3S was comparable to that generated by AAV-Cre with a 10-fold lower MOI (FIG. 14D). The lower fluorescent intensity of tdTomato in the RAAV-infected cells was possibly due to the short lifetime of the Cre mRNAs delivered thereinto and the inability of the limited amount of the translated Cre recombinase to rescue both of the two copies of tdTomato expression cassettes in the homo-Ai9-MEF cells (FIG. 16).

By comparing the results of the DNA titer and cytometric data, it was indicated that the majority of the tdTomato red fluorescence signals in the RAAV-CCWM3S infected cells were generated by the Cre mRNA-harboring RAAV particles.

In conclusion, the inventive RAAV vector could deliver functional Cre mRNAs into cells and express functional Cre recombinase.

### Example 13: In Vitro Transient Transfer of Functional Gene by RAAV Particles into Cells

To determine the exact lifespan of the Cre protein produced via AAV-Cre or RAAV-CCWM3S delivery as in Example 7, Ai9-MEF cells were seeded 24 hrs before infection at a cell confluence of 5×10⁴ cells per well, and then incubated with AAV-Cre (MOI=300) or RAAV-CCWM3S (MOI=10,000) vectors overnight as described above. After infection, cells were collected at several time points, and fluorescence photos were taken prior to the cell collection.

For AAV-Cre, Cre expression increased during the first 4 days, but then decreased. By contrast, a small amount of Cre was detected at as early as about 24 hrs after RAAV-CCWM3S transfer and disappeared after Day 2. This quick expression and degradation phenomenon may be due to the instant appearance and short lifetime of the delivered functional Cre mRNA (FIGS. 17A and 17B).

### Example 14: In Vivo Transient Transfer of Functional Gene by RAAV Particles into Ai9-Mouse

This example demonstrates that the RAAV particles can be used as a tool for in vivo gene delivery and to express the functional Cre recombinase transiently.

To investigate the infectivity of RAAV particles in vivo, Ai9-Mice (2.5-4 months old) were anesthetized and injected with 1 μL AAV-Cre (pssAAV-Cre/DJ) (high dose: 1E9 vg/mouse; low dose: 3E6 vg/mouse) or 1 μL RAAV-Cre (L-CCWM3S/DJ-MCP (Y156F)) (1E9 vg/mouse) into the right hippocampus according to the following coordinates: anteroposterior (A/P) ~1.7 mm, mediolateral (M/L) ~1.0 mm, dorsoventral (D/V) ~2.1 mm. Also, AAV capsid-DJ was used in this assay as a control.

Six weeks after AAV or RAAV injection, mice were anesthetized and transcardially perfused with PBS at room temperature at pH 7.4 and then with freshly prepared, ice-cold 4% paraformaldehyde (PFA) in phosphate buffers (PB). The brains were post-fixed in 4% PFA overnight. The fixed brains were embedded with OCT for frozen section after dehydration. Brains were sectioned in 20 μm thickness using a freezing microtome (Leica CM1950), and the sections were mounted to slides directly. The slides were baked at 60° C. for 1-2 hours followed by blocking with 5% BSA serum in PBS for 1 h. Subsequently, the slides were incubated with the primary antibody against Cre (10536; Cell Signaling Technology; 1:800 dilution) in 5% BSA in PBS (0.1% Triton-X) overnight at room temperature. After five washes with PBS, the slides were incubated in 1% BSA in PBS containing secondary antibody against the primary antibody and DAPI (D3571, Invitrogen). The secondary antibody used was Alexa Fluor 488 donkey anti-rabbit IgG (711-545-152, Jackson ImmunoResearch) (at 1:1000 dilution). Images were acquired with Nikon C2si+ Confocal Microscope.

The acquired images showing fluorescence from tdTomato expression system demonstrated that RAAV-Cre infected the cells in Ai9-mice hippocampus and rescued the expression of tdTomato. The number of the infected cells in the RAAV-Cre group was less than that of the AAV-Cre group at the same dose. However, the RAAV-Cre infection generated much more tdTomato positive cells relative to the low-dose group (30-fold lower dose) of AAV-Cre infection.

Very interestingly, the Cre expression was easily detected in both the high-dose and low-dose groups of AAV-Cre infection, but no significant Cre expression was detected in the RAAV-Cre infected cells despite of the detected tdTomato fluorescence proving the once existence of Cre.

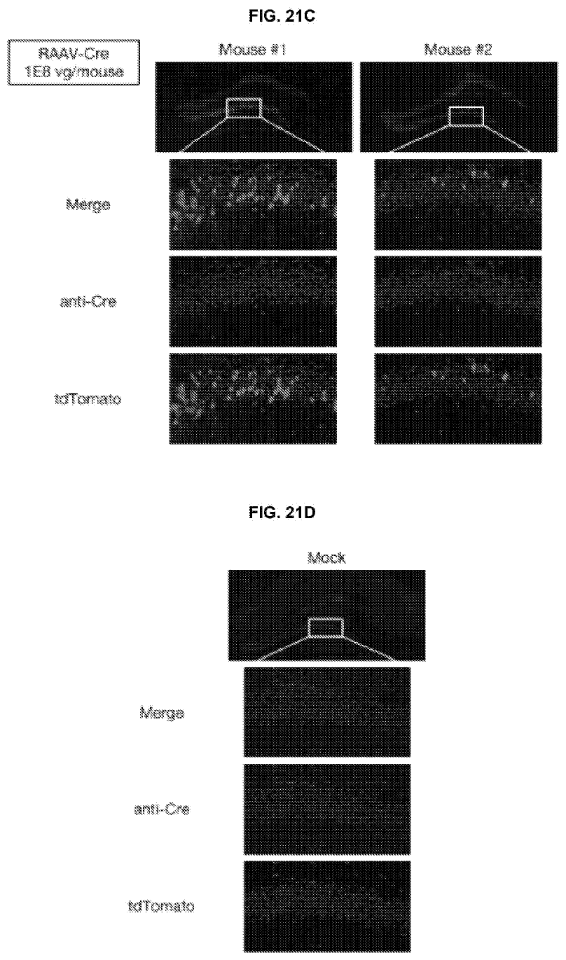

Overall, the RAAV-Cre had an inferior transduction efficiency compared to the conventional AAV-Cre as shown by the fluorescent photos (positive cell counts) for the two at the same high dose of 1E9 vg/mouse (FIG. 21A vs. FIG. 21C), since multiple mRNAs for protein translation can be transcribed from one successfully transduced AAV DNA genome. To further evaluate the Cre expression levels, the transduction efficiency of the AAV-Cre was normalized to that of the RAAV-Cre by reducing the high dose of AAV-Cre of 1E9 vg/mouse to a low dose of 3E6 vg/mouse (FIG. 21B), and the results showed that although the RAAV-Cre had a superior transduction efficiency to the low dose group of AAV-Cre as shown by the fluorescent photos (positive cell counts), no Cre expression was detected in the RAAV-Cre infected cells as compared with the AAV-Cre group, which indicated that the Cre expression in RAAV-Cre group was transient but functional.

### Example 15: Additional RPS/RBP Pairs for RAAV System

In addition to the MS2/MCP pair used in Examples 3-8, two additional pairs of RNA aptamer/aptamer-binding proteins (or RNA packaging signal/RNA binding protein, “RPS/RBP” herein) were tested for RAAV packaging: (1) PP7 binding site/PP7 bacteriophage coat protein (“PP7/PCP,” or “PCP” or “P” for short, or “P” in L-CCWP3S) and (2) Com binding site/phage COM protein (“com/COM,” or “COM” for short, or “C” in L-CCWC3S). Unlike MS2/MCP and PP7/PCP that are natural viral packaging systems, com/COM is not a natural viral packaging system but known to be transcription regulators that play roles in the transcription initiation of the bacteriophage Mu mom gene.

Transgene plasmids harboring three copies of RPS (L-CCWP3S and L-CCWC3S) and their corresponding packaging plasmids [DJ-PCP (Y156F) with PP7 bacteriophage coat protein (PCP) fused to the N-terminus of Rep78-Y156F and Rep68-Y156F and DJ-COM (Y156F) with phage COM protein (COM) fused to the N-terminus of Rep78-Y156F and Rep68-Y156F] were constructed. Viral particles were produced, purified, and titrated as described in Example 3.

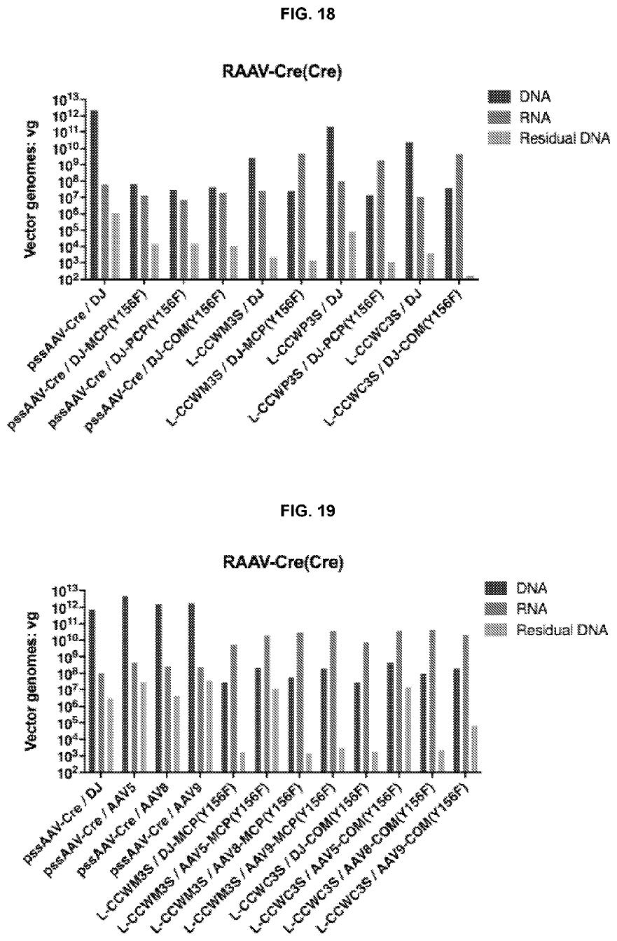

The results showed that similar to the MS2/MCP pair well demonstrated in various aspects in Examples 3-8, the two pairs of PP7/PCP and com/COM also led to the remarkable RNA packaging of RAAV particles (FIG. 18), thereby expanding the scope of various RAAV packaging system.

### Example 16: Application of RAAV System to Various AAV Serotypes

To investigate the application of the inventive RAAV packaging system to various AAV serotypes in addition to AAV-DJ tested in Examples 3-9, two pairs of RPS/RBP (MS2/MCP and com/COM) were examined in AAV-DJ and another three different AAV serotypes (AAV5, AAV8 and AAV9). Viral particles were produced, purified, and titrated as described in Example 3.

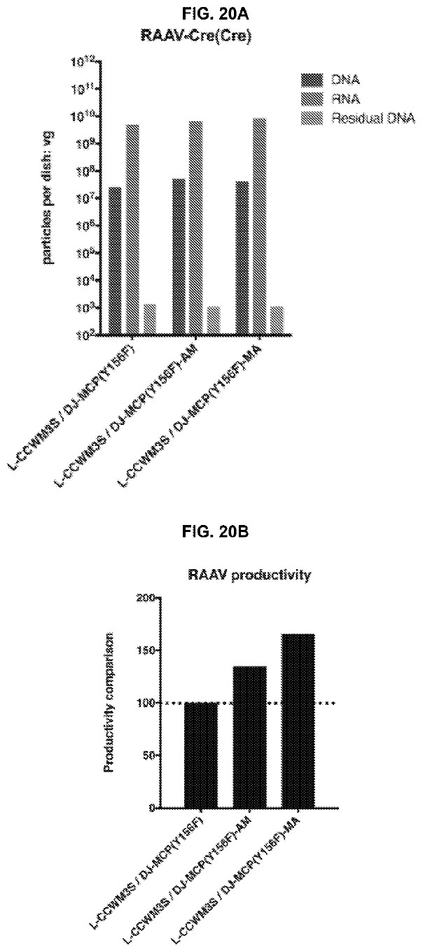

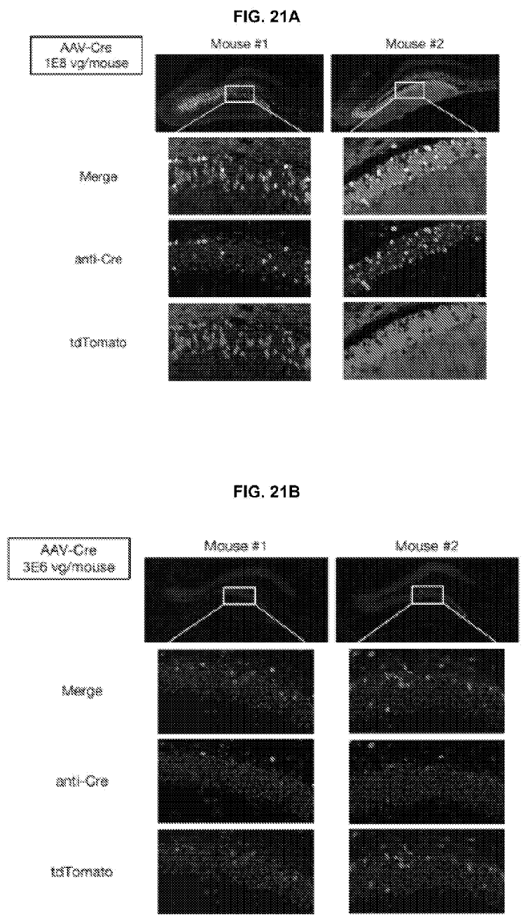

Both RAAV-MS2/MCP and RAAV-com/COM system worked well in all the four serotypes, suggesting the general applicability of the RAAV packaging systems to different AAV serotypes (i.e., not limited to AAV-DJ). In the presence of the RBP, Cre RNA genomes containing the corresponding RPS were efficiently encapsidated into the respective RAAV particles. Though the yields of the RNA-packaged RAAV particles varied from serotype to serotype, all of the RAAV5, RAAV8 and RAAV9 particles had a higher productivity than RAAV-DJ (FIG. 19).

### Example 17: AAP and MCP Fusion Protein Increased RAAV Yield

Generally, AAV encodes a unique assembly-activating protein (AAP) within their natural viral genomes that is essential for capsid assembly. Specifically, AAP was found to be essential for capsid protein stabilization and generation of functional AAV particles.

An AAP-MCP (with MCP fused to the C-terminus of AAP) or MCP-AAP (with MCP fused to the N-terminus of AAP) fusion protein expression cassette was inserted inversely into the backbone of the packaging plasmid DJ-MCP (Y156F) used in Examples 6-10, and the resulting constructs were named DJ-MCP(Y156F)-AM and DJ-MCP(Y156F)-MA, respectively. Such constructs then expressed both MCP-Rep78/68(Y156F) fusion and AAP-MCP or MCP-AAP fusion, increasing the amount of RNA binding proteins (RBPs) assisting in RNA packaging compared with MCP-Rep78/68 fusion alone. Viral particles were produced, purified, and titrated as described in Example 3.

The results showed that the yields of RNA-packaged RAAV particles were increased by about 65% in DJ-MCP (Y156F)-MA and about 35% in DJ-MCP (Y156F)-AM compared with MCP-Rep78/68 fusion alone (FIGS. 20A and 20B), suggesting that RBPs could be additionally fused to or associated with any other proteins which play roles in the packaging or assembly of AAV particles in order to enhance the RNA packaging of RAAV particles.

Using AAP-MCP or MCP-AAP alone, without MCP-Rep78/68, are also within the scope of the disclosure.

Various modifications and variations of the described products, methods, and uses of the disclosure will be apparent to those skilled in the art without departing from the scope and spirit of the disclosure. Although the disclosure has been described in connection with specific embodiments, it will be understood that it is capable of further modifications and that the disclosure as claimed should not be unduly limited to such specific embodiments. Indeed, various modifications of the described modes for carrying out the disclosure that are obvious to those skilled in the art are intended to be within the scope of the disclosure. This application is intended to cover any variations, uses, or adaptations of the disclosure following, in general, the principles of the disclosure and including such departures from the disclosure come within known customary practice within the art to which the disclosure pertains and may be applied to the essential features herein before set forth.

### REFERENCES, WHICH ARE INCORPORATED HEREIN BY REFERENCE IN THEIR ENTIRETY

1. X. Hou, T. Zaks, R. Langer, Y. Dong, Lipid nanoparticles for mRNA delivery. Nat Rev Mater 6, 1078-1094 (2021).
2. A. Akinc et al., The Onpattro story and the clinical translation of nanomedicines containing nucleic acid-based drugs. Nat Nanotechnol 14, 1084-1087 (2019).
3. E. J. Anderson et al., Safety and Immunogenicity of SARS-CoV-2 mRNA-1273 Vaccine in Older Adults. N Engl J Med 383, 2427-2438 (2020).
4. L. R. Baden et al., Efficacy and Safety of the mRNA-1273 SARS-CoV-2 Vaccine. N Engl J Med 384, 403-416 (2021).
5. F. P. Polack et al., Safety and Efficacy of the BNT162b2 mRNA Covid-19 Vaccine. N Engl J Med 383, 2603-2615 (2020).
6. P. Lyu, L. Wang, B. Lu, Virus-Like Particle Mediated CRISPR/Cas9 Delivery for Efficient and Safe Genome Editing. Life (Basel) 10, (2020).
7. M. Segel et al., Mammalian retrovirus-like protein PEG10 packages its own mRNA and can be pseudotyped for mRNA delivery. Science 373, 882-889 (2021).
8. S. Ling et al., Lentiviral delivery of co-packaged Cas9 mRNA and a Vegfa-targeting guide RNA prevents wet age-related macular degeneration in mice. Nat Biomed Eng 5, 144-156 (2021).
9. Y. Knopp et al., Transient Retrovirus-Based CRISPR/Cas9 All-in-One Particles for Efficient, Targeted Gene Knockout. Mol Ther Nucleic Acids 13, 256-274 (2018).
10. B. Lu et al., Delivering SaCas9 mRNA by lentivirus-like bionanoparticles for transient expression and efficient genome editing. Nucleic Acids Res 47, e44 (2019).
11. A. Raguram, S. Banskota, D. R. Liu, Therapeutic in vivo delivery of gene editing agents. Cell 185, 2806-2827 (2022).
12. D. Goertsen et al., AAV capsid variants with brain-wide transgene expression and decreased liver targeting after intravenous delivery in mouse and marmoset. Nat Neurosci 25, 106-115 (2022).
13. M. Tabebordbar et al., Directed evolution of a family of AAV capsid variants enabling potent muscle-directed gene delivery across species. Cell 184, 4919-4938.e4922 (2021).
14. L. W. Koblan et al., In vivo base editing rescues Hutchinson-Gilford progeria syndrome in mice. Nature 589, 608-614 (2021).
15. R. J. Samulski, N. Muzyczka, AAV-Mediated Gene Therapy for Research and Therapeutic Purposes. Annu Rev Virol 1, 427-451 (2014).
16. D. Wang, P. W. L. Tai, G. Gao, Adeno-associated virus vector as a platform for gene therapy delivery. Nat Rev Drug Discov 18, 358-378 (2019).
17. J. A. King, R. Dubielzig, D. Grimm, J. A. Kleinschmidt, DNA helicase-mediated packaging of adeno-associated virus type 2 genomes into preformed capsids. Embo j 20, 3282-3291 (2001).
18. A. C. Maurer, M. D. Weitzman, Adeno-Associated Virus Genome Interactions Important for Vector Production and Transduction. Hum Gene Ther 31, 499-511 (2020).
19. A. Prel et al., Highly efficient in vitro and in vivo delivery of functional RNAs using new versatile MS2-chimeric retrovirus-like particles. Mol Ther Methods Clin Dev 2, 15039 (2015).
20. Y. Baron et al., Improved alpharetrovirus-based Gag.MS2 particles for efficient and transient delivery of CRISPR-Cas9 into target cells. Mol Ther Nucleic Acids 27, 810-823 (2022).
21. M. Yadav, A. Atala, B. Lu, Developing all-in-one virus-like particles for Cas9 mRNA/single guide RNA co-delivery and aptamer-containing lentiviral vectors for improved gene expression. Int J Biol Macromol 209, 1260-1270 (2022).
22. M. D. Davis, J. Wu, R. A. Owens, Mutational analysis of adeno-associated virus type 2 Rep68 protein endonuclease activity on partially single-stranded substrates. J Virol 74, 2936-2942 (2000).
23. F. Lim, T. P. Downey, D. S. Peabody, Translational repression and specific RNA binding by the coat protein of the Pseudomonas phage PP7. J Biol Chem 276, 22507-22513 (2001).
24. F. G. Wulczyn, R. Kahmann, Translational stimulation: RNA sequence and structure requirements for binding of Com protein. Cell 65, 259-269 (1991).
25. V. Santosh et al., The Cryo-EM structure of AAV2 Rep68 in complex with ssDNA reveals a malleable AAA+ machine that can switch between oligomeric states. Nucleic Acids Res 48, 12983-12999 (2020).
26. M. R. Singleton, M. S. Dillingham, D. B. Wigley, Structure and mechanism of helicases and nucleic acid translocases. Annu Rev Biochem 76, 23-50 (2007).
27. L. M. Iyer, L. Aravind, E. V. Koonin, Common origin of four diverse families of large eukaryotic DNA viruses. J Virol 75, 11720-11734 (2001).
28. L. M. Iyer, D. D. Leipe, E. V. Koonin, L. Aravind, Evolutionary history and higher order classification of AAA+ ATPases. J Struct Biol 146, 11-31 (2004).
29. C. Li, R. J. Samulski, Engineering adeno-associated virus vectors for gene therapy. Nat Rev Genet 21, 255-272 (2020).
30. Y. Yu et al., Pwp1 regulates telomere length by stabilizing shelterin complex and maintaining histone H4K20 trimethylation. Cell Discov 5, 47 (2019).
31. S. O. Sutter et al., Adeno-associated virus type 2 (AAV2) uncoating is a stepwise process and is linked to structural reorganization of the nucleolus. PLoS Pathog 18, e1010187 (2022).
32. J. M. Halstead et al., Translation. An RNA biosensor for imaging the first round of translation from single cells to living animals. Science 347, 1367-1671 (2015).
33. H. Zhang et al., An engineered xCas12i with high activity, high specificity and broad PAM range. Protein & Cell, (2022).
34. J. Yin et al., Safeguarding genome integrity during gene-editing therapy in a mouse model of age-related macular degeneration. Nat Commun 13, 7867 (2022).
35. R. Col&1-Thillet, K. R. Jerome, D. Stone, Optimization of AAV vectors to target persistent viral reservoirs. Virol J 18, 85 (2021).
36. K. Y. Chan et al., Engineered AAVs for efficient noninvasive gene delivery to the central and peripheral nervous systems. Nat Neurosci 20, 1172-1179 (2017).
37. S. Ravindra Kumar et al., Multiplexed Cre-dependent selection yields systemic AAVs for targeting distinct brain cell types. Nat Methods 17, 541-550 (2020).

### SEQUENCES

Certain sequences, including those referenced in the examples above, are provided herein below.

TABLE A
Nucleic Acid Sequence and Amino Acid Sequence of RPS-binding protein

| RPS-binding protein | Sequences |
|---|---|
| MCP nucleic acid | gcttctaactttactcagttcgttctcgtcgacaatggcggaactggcgacgtgactgtegccccaagcaac |
| sequence | ttcgctaacggggtcgctgaatggatcagctctaactcgcgttcacaggcttacaaagtaacctgtagcgtt |
| SEQ ID NO: 48 | cgtcagagctctgcgcagaatcgcaaatacaccatcaaagtcgaggtgcctaaagtggcaacccagactgtt |
| | ggtggagtagagcttcctgtagccgcatggcgttcgtacttaaatatggaactaaccattccaattttcgct |
| | acgaattccgactgcgagcttattgttaaggcaatgcaaggtctcctaaaagatggaaacccgattccctca |
| | gcaatcgcagcaaactccggcatctac |
| MCP amino acid | ASNFTQFVLVDNGGTGDVTVAPSNFANGVAEWISSNSRSQAYKVTCSVRQ |
| sequence | SSAQNRKYTIKVEVPKVATQTVGGVELPVAAWRSYLNMELTIPIFATNSDCE |
| SEQ ID NO: 49 | LIVKAMQGLLKDGNPIPSAIAANSGIY |
| PCP nucleic acid | tccaaaacaatagtcctctccgtaggggaggcaacacggactttgaccgaaatccagtcaaccgctgaccga |
| sequence | caaatctttgaagagaaagtagggcctcttgtgggccgactgcgcttgactgcaagcttgcgacaaaacggc |
| SEQ ID NO: 50 | gcaaagactgcctatagggtcaaccttaaactcgaccaagccgacgtggtcgatagcggtctccctaaggtt |
| | cggtatacgcaggtctggagtcatgacgtaacaatcgtagcaaacagcacagaagcctcccgaaaaagcctc |
| | tacgatctgacgaaatccttggtggctacgtcacaggtggaagacctcgttgtcaaccttgtacctctgggt |
| | cga |
| PCP amino acid | SKTIVLSVGEATRTLTEIQSTADRQIFEEKVGPLVGRLRLTASLRQNGAKTAY |
| sequence | RVNLKLDQADVVDSGLPKVRYTQVWSHDVTIVANSTEASRKSLYDLTKSL |
| SEQ ID NO: 51 | VATSQVEDLVVNLVPLGR |
| COM nucleic acid | atgaaatcaattcgctgtaaaaactgcaacaaactgttatttaaggcggattcctttgatcacattgaaatc |
| sequence | aggtgtccgcgttgcaaacgtcacatcataatgctgaatgcctgcgagcatcccacggagaaacattgtggg |
| SEQ ID NO: 52 | aaaagagaaaaaatcacgcattctgacgaaaccgtgcgttat |
| COM amino acid | MKSIRCKNCNKLLFKADSFDHIEIRCPRCKRHIIMLNACEHPTEKHCGKRE |
| sequence | KITHSDETVRY |
| SEQ ID NO: 53 | |

TABLE B
Nucleic Acid Sequences of RNA packaging signal (RPS)

| Packaging signal | Nucleic acid sequences |
|---|---|
| MS2 | acatgaggatcacccatgt |
| SEQ ID NO: 54 | |
| MS2 × 3 (MS2- | acatgaggatcacccatgtctgcaggtcgactctagaaaacatgaggatcacccatgtctgcagtattcccgg |
| linker-MS2- | gttcattagatcctaaggtacctaattgcctagaaaacatgaggatcacccatgt |
| linker-MS2) | |
| SEQ ID NO: 55 | |
| PP7 | GGAGCAGACGATATGGCGTCGCTCC |
| SEQ ID NO: 56 | |
| PP7 × 3 (PP7- | GGAGCAGACGATATGGCGTCGCTCCctgcaggtcgactctagaaaGGAGCAGAC |
| linker-PP7- | GATATGGCGTCGCTCCctgcagtattcccgggttcattagatcctaaggtacctaattgcctagaaa |
| linker-PP7) | GGAGCAGACGATATGGCGTCGCTCC |
| SEQ ID NO: 57 | |
| com | GAATGCCTGCGAGCATCC |
| SEQ ID NO: 58 | |
| com × 3 (com- | GAATGCCTGCGAGCATCCctgcaggtcgactctagaaaGAATGCCTGCGAGCATC |
| linker-com- | CctgcagtattcccgggttcattagatcctaaggtacctaattgcctagaaaGAATGCCTGCGAGCA |
| linker-com) | TCC |
| SEQ ID NO: 59 | |

*Sequence elements are matched based on formatting styles (e.g., bold and/or italic fonts, etc.)

TABLE C
Nucleic Acid Sequence and Amino Acid Sequence of Rep proteins

| Rep proteins | Sequences |
|---|---|
| Rep2 (Rep78 from | ATGCCGGGGTTTTACGAGATTGTGATTAAGGTCCCCAGCGACCTTGACG |
| AAV2) nucleic acid | AGCATCTGCCCGGCATTTCTGACAGCTTTGTGAACTGGGTGGCCGAGAA |
| sequence | GGAATGGGAGTTGCCGCCAGATTCTGACATGGATCTGAATCTGATTGAG |
| SEQ ID NO: 60 | CAGGCACCCCTGACCGTGGCCGAGAAGCTGCAGCGCGACTTTCTGACG |
| | GAATGGCGCCGTGTGAGTAAGGCCCCGGAGGCCCTTTTCTTTGTGCAAT |
| | TTGAGAAGGGAGAGAGCTACTTCCACATGCACGTGCTCGTGGAAACCA |
| | CCGGGGTGAAATCCATGGTTTTGGGACGTTTCCTGAGTCAGATTCGCGA |
| | AAAACTGATTCAGAGAATTTACCGCGGGATCGAGCCGACTTTGCCAAAC |
| | TGGTTCGCGGTCACAAAGACCAGAAATGGCGCCGGAGGCGGGAACaaG |
| | GTGGTGGaTGAGTGCTACATCCCCAATTaCTTGCTCCCCAAAACCCAGCC |
| | TGAGCTCCAGTGGGCGTGGACTAATATGGAACAGTATTTAAGCGCCTGT |
| | TTGAATCTCACGGAGCGTAAACGGTTGGTGGCGCAGCATCTGACGCAC |
| | GTGTCGCAGACGCAGGAGCAGAACAAAGAGAATCAGAATCCCAATTCT |
| | GATGCGCCGGTGATCAGATCAAAAACTTCAGCCAGGTACATGGAGCTGG |
| | TCGGGTGGCTCGTGGACAAGGGGATTACCTCGGAGAAGCAGTGGATCC |
| | AGGAGGACCAGGCCTCATACATCTCCTTCAATGCGGCCTCCAACTCGCG |
| | GTCCCAAATCAAGGCTGCCTTGGACAATGCGGGAAAGATTATGAGCCTG |
| | ACTAAAACCGCCCCCGACTACCTGGTGGGCCAGCAGCCCGTGGAGGAC |
| | ATTTCCAGCAATCGGATTTATAAAATTTTGGAACTAAACGGGTACGATCC |
| | CCAATATGCGGCTTCCGTCTTTCTGGGATGGGCCACGAAAAAGTTCGGC |
| | AAGAGGAACACCATCTGGCTGTTTGGGCCTGCAACTACCGGGAAGACC |
| | AACATCGCGGAGGCCATAGCCCACACTGTGCCCTTCTACGGGTGCGTAA |
| | ACTGGACCAATGAGAACTTTCCCTTCAACGACTGTGTCGACAAGATGGT |
| | GATCTGGTGGGAGGAGGGGAAGATGACCGCCAAGGTCGTGGAGTCGGC |
| | CAAAGCCATTCTCGGAGGAAGCAAGGTGCGCGTGGACCAGAAATGCAA |
| | GTCCTCGGCCCAGATAGACCCGACTCCCGTGATCGTCACCTCCAACACC |
| | AACATGTGCGCCGTGATTGACGGGAACTCAACGACCTTCGAACACCAG |
| | CAGCCGTTGCAAGACCGGATGTTCAAATTTGAACTCACCCGCCGTCTGG |
| | ATCATGACTTTGGGAAGGTCACCAAGCAGGAAGTCAAAGACTTTTTCCG |
| | GTGGGCAAAGGATCACGTGGTTGAGGTGGAGCATGAATTCTACGTCAA |
| | AAAGGGTGGAGCCAAGAAAAGACCCGCCCCCAGTGACGCAGATATAAG |
| | TGAGCCCAAACGGGTGCGCGAGTCAGTTGCGCAGCCATCGACGTCAGA |
| | CGCGGAAGCTTCGATCAACTACGCAGACAGGTACCAAAACAAATGTTCT |
| | CGTCACGTGGGCATGAATCTGATGCTGTTTCCCTGCAGACAATGCGAGA |
| | GAATGAATCAGAATTCAAATATCTGCTTCACTCACGGACAGAAAGACTG |
| | TTTAGAGTGCTTTCCCGTGTCAGAATCTCAACCCGTTTCTGTCGTCAAA |
| | AAGGCGTATCAGAAACTGTGCTACATTCATCATATCATGGGAAAGGTGC |
| | CAGACGCTTGCACTGCCTGCGATCTGGTCAATGTGGATTTGGATGACTG |
| | CATCTTTGAACAATAA |
| Rep2 (Rep78 from | MPGFYEIVIKVPSDLDEHLPGISDSFVNWVAEKEWELPPDSDMDLNLIEQA |
| AAV2) amino acid | PLTVAEKLQRDFLTEWRRVSKAPEALFFVQFEKGESYFHMHVLVETTGVKS |
| sequence | MVLGRFLSQIREKLIQRIYRGIEPTLPNWFAVTKTRNGAGGGNKVVDECYI |
| SEQ ID NO: 61 | PNYLLPKTQPELQWAWTNMEQYLSACLNLTERKRLVAQHLTHVSQTQEQN |
| | KENQNPNSDAPVIRSKTSARYMELVGWLVDKGITSEKQWIQEDQASYISFN |
| | AASNSRSQIKAALDNAGKIMSLIKTAPDYLVGQQPVEDISSNRIYKILELNG |
| | YDPQYAASVFLGWATKKFGKRNTIWLFGPATTGKTNIAEAIAHTVPFYGCV |
| | NWTNENFPFNDCVDKMVIWWEEGKMTAKVVESAKAILGGSKVRVDQKC |
| | KSSAQIDPTPVIVTSNTNMCAVIDGNSTTFEHQQPLQDRMFKFELTRRLDH |
| | DFGKVTKQEVKDFFRWAKDHVVEVEHEFYVKKGGAKKRPAPSDADISEP |
| | KRVRESVAQPSTSDAEASINYADRYQNKCSRHVGMNLMLFPCRQCERMN |
| | QNSNICFTHGQKDCLECFPVSESQPVSVVKKAYQKLCYIHHIMGKVPDAC |
| | TACDLVNVDLDDCIFEQ |
| MCP-Rep2 nucleic | ATGCCcGGCAGCTCCGGCAGTAGCgcttctaactttactcagttcgttctcgtcgacaatggcgg |
| acid sequence (MP- | aactggcgacgtgactgtcgccccaagcaacttcgctaacggggtcgctgaatggatcagctctaactcg |
| linker-MCP-linker- | cgttcacaggcttacaaagtaacctgtagcgttcgtcagagctctgcgcagaatcgcaaatacaccatca |
| remaining Rep2) | aagtcgaggtgcctaaagtggcaacccagactgttggtggagtagagcttcctgtagccgcatggcgttc |
| SEQ ID NO: 62 | gtacttaaatatggaactaaccattccaattttcgctacgaattccgactgcgagcttattgttaaggca |
| | atgcaaggtctcctaaaagatggaaacccgattccctcagcaatcgcagcaaactccggcatctac |
| | GGCAGTAGTGGGTCCTCTGGGTTTTACGAGATTGTGATTAAGGTCCCCAGCGACCTTGACGAGCATCTGCC |
| | CGGCATTTCTGACAGCTTTGTGAACTGGGTGGCCGAGAAGGAATGGGA |
| | GTTGCCGCCAGATTCTGACATGGATCTGAATCTGATTGAGCAGGCACCC |
| | CTGACCGTGGCCGAGAAGCTGCAGCGCGACTTTCTGACGGAATGGCGC |
| | CGTGTGAGTAAGGCCCCGGAGGCCCTTTTCTTTGTGCAATTTGAGAAGG |
| | GAGAGAGCTACTTCCACATGCACGTGCTCGTGGAAACCACCGGGGTGA |
| | AATCCATGGTTTTGGGACGTTTCCTGAGTCAGATTCGCGAAAAACTGAT |
| | TCAGAGAATTTACCGCGGGATCGAGCCGACTTTGCCAAACTGGTTCGCG |
| | GTCACAAAGACCAGAAATGGCGCCGGAGGCGGGAACAAGGTGGTGGA |
| | TGAGTGCTACATCCCCAATTACTTGCTCCCCAAAACCCAGCCTGAGCTC |
| | CAGTGGGCGTGGACTAATATGGAACAGTATTTAAGCGCCTGTTTGAATCT |
| | CACGGAGCGTAAACGGTTGGTGGCGCAGCATCTGACGCACGTGTCGCA |
| | GACGCAGGAGCAGAACAAAGAGAATCAGAATCCCAATTCTGATGCGCC |
| | GGTGATCAGATCAAAAACTTCAGCCAGGTACATGGAGCTGGTCGGGTG |
| | GCTCGTGGACAAGGGGATTACCTCGGAGAAGCAGTGGATCCAGGAGGA |
| | CCAGGCCTCATACATCTCCTTCAATGCGGCCTCCAACTCGCGGTCCCAA |
| | ATCAAGGCTGCCTTGGACAATGCGGGAAAGATTATGAGCCTGACTAAAA |
| | CCGCCCCCGACTACCTGGTGGGCCAGCAGCCCGTGGAGGACATTTCCA |
| | GCAATCGGATTTATAAAATTTTGGAACTAAACGGGTACGATCCCCAATAT |
| | GCGGCTTCCGTCTTTCTGGGATGGGCCACGAAAAAGTTCGGCAAGAGG |
| | AACACCATCTGGCTGTTTGGGCCTGCAACTACCGGGAAGACCAACATCG |
| | CGGAGGCCATAGCCCACACTGTGCCCTTCTACGGGTGCGTAAACTGGAC |
| | CAATGAGAACTTTCCCTTCAACGACTGTGTCGACAAGATGGTGATCTGG |
| | TGGGAGGAGGGGAAGATGACCGCCAAGGTCGTGGAGTCGGCCAAAGC |
| | CATTCTCGGAGGAAGCAAGGTGCGCGTGGACCAGAAATGCAAGTCCTC |
| | GGCCCAGATAGACCCGACTCCCGTGATCGTCACCTCCAACACCAACATG |
| | TGCGCCGTGATTGACGGGAACTCAACGACCTTCGAACACCAGCAGCCG |
| | TTGCAAGACCGGATGTTCAAATTTGAACTCACCCGCCGTCTGGATCATG |
| | ACTTTGGGAAGGTCACCAAGCAGGAAGTCAAAGACTTTTTCCGGTGGG |
| | CAAAGGATCACGTGGTTGAGGTGGAGCATGAATTCTACGTCAAAAAGG |
| | GTGGAGCCAAGAAAAGACCCGCCCCCAGTGACGCAGATATAAGTGAGC |
| | CCAAACGGGTGCGCGAGTCAGTTGCGCAGCCATCGACGTCAGACGCGG |
| | AAGCTTCGATCAACTACGCAGACAGGTACCAAAACAAATGTTCTCGTCA |
| | CGTGGGCATGAATCTGATGCTGTTTCCCTGCAGACAATGCGAGAGAATG |
| | AATCAGAATTCAAATATCTGCTTCACTCACGGACAGAAAGACTGTTTAG |
| | AGTGCTTTCCCGTGTCAGAATCTCAACCCGTTTCTGTCGTCAAAAAGGC |
| | GTATCAGAAACTGTGCTACATTCATCATATCATGGGAAAGGTGCCAGAC |
| | GCTTGCACTGCCTGCGATCTGGTCAATGTGGATTTGGATGACTGCATCTT |
| | TGAACAATAA |
| MCP-Rep2 amino acid | MPGSSGSSASNFTQFVLVDNGGTGDVTVAPSNFANGVAEWISSNSRSQAYK |
| sequence (MP- | VTCSVRQSSAQNRKYTIKVEVPKVATQTVGGVELPVAAWRSYLNMELTIPIF |
| linker-MCP-linker- | ATNSDCELIVKAMQGLLKDGNPIPSAIAANSGIYGSSGSSGFYEIVIKVPSDL |
| remaining Rep2) | DEHLPGISDSFVNWVAEKEWELPPDSDMDLNLIEQAPLTVAEKLQRDFLTE |
| SEQ ID NO: 63 | WRRVSKAPEALFFVQFEKGESYFHMHVLVETTGVKSMVLGRFLSQIREKLI |
| | QRIYRGIEPTLPNWFAVTKTRNGAGGGNKVVDECYIPNYLLPKTQPELQW |
| | AWTNMEQYLSACLNLTERKRLVAQHLTHVSQTQEQNKENQNPNSDAPVIR |
| | SKTSARYMELVGWLVDKGITSEKQWIQEDQASYISFNAASNSRSQIKAALD |
| | NAGKIMSLTKTAPDYLVGQQPVEDISSNRIYKILELNGYDPQYAASVFLGW |
| | ATKKFGKRNTIWLFGPATTGKTNIAEAIAHTVPFYGCVNWTNENFPFNDCV |
| | DKMVIWWEEGKMTAKVVESAKAILGGSKVRVDQKCKSSAQIDPTPVIVTS |
| | NTNMCAVIDGNSTTFEHQQPLQDRMFKFELTRRLDHDFGKVTKQEVKDFF |
| | RWAKDHVVEVEHEFYVKKGGAKKRPAPSDADISEPKRVRESVAQPSTSDA |
| | EASINYADRYQNKCSRHVGMNLMLFPCRQCERMNQNSNICFTHGQKDCL |
| | ECFPVSESQPVSVVKKAYQKLCYIHHIMGKVPDACTACDLVNVDLDDCIFE |
| | Q |
| MCP-Rep2-Y156F | ATGCCcGGCAGCTCCGGCAGTAGCgcttctaactttactcagttcgttctcgtcgacaatggcgg |
| nucleic acid | aactggcgacgtgactgtcgccccaagcaacttcgctaacggggtcgctgaatggatcagctctaactcg |
| sequence | cgttcacaggcttacaaagtaacctgtagcgttcgtcagagctctgcgcagaatcgcaaatacaccatca |
| (MP-linker-MCP- | aagtcgaggtgcctaaagtggcaacccagactgttggtggagtagagcttcctgtagccgcatggcgttc |
| linker-remaining | gtacttaaatatggaactaaccattccaattttcgctacgaattccgactgcgagcttattgttaaggca |
| Rep2) | atgcaaggtctcctaaaagatggaaacccgattccctcagcaatcgcagcaaactccggcatctac |
| SEQ ID NO: 64 | GGCAGTAGTGGGTCCTCTGGGTTTTACGAGATTGTGATTAAGGTCCCCAGCGACCTTGACGAGCATCTGCC |
| | CGGCATTTCTGACAGCTTTGTGAACTGGGTGGCCGAGAAGGAATGGGA |
| | GTTGCCGCCAGATTCTGACATGGATCTGAATCTGATTGAGCAGGCACCC |
| | CTGACCGTGGCCGAGAAGCTGCAGCGCGACTTTCTGACGGAATGGCGC |
| | CGTGTGAGTAAGGCCCCGGAGGCCCTTTTCTTTGTGCAATTTGAGAAGG |
| | GAGAGAGCTACTTCCACATGCACGTGCTCGTGGAAACCACCGGGGTGA |
| | AATCCATGGTTTTGGGACGTTTCCTGAGTCAGATTCGCGAAAAACTGAT |
| | TCAGAGAATTTACCGCGGGATCGAGCCGACTTTGCCAAACTGGTTCGCG |
| | GTCACAAAGACCAGAAATGGCGCCGGAGGCGGGAACAAGGTGGTGGA |
| | TGAGTGCTACATCCCCAATTtCTTGCTCCCCAAAACCCAGCCTGAGCTCC |
| | AGTGGGCGTGGACTAATATGGAACAGTATTTAAGCGCCTGTTTGAATCTC |
| | ACGGAGCGTAAACGGTTGGTGGCGCAGCATCTGACGCACGTGTCGCAG |
| | ACGCAGGAGCAGAACAAAGAGAATCAGAATCCCAATTCTGATGCGCCG |
| | GTGATCAGATCAAAAACTTCAGCCAGGTACATGGAGCTGGTCGGGTGG |
| | CTCGTGGACAAGGGGATTACCTCGGAGAAGCAGTGGATCCAGGAGGAC |
| | CAGGCCTCATACATCTCCTTCAATGCGGCCTCCAACTCGCGGTCCCAAAT |
| | CAAGGCTGCCTTGGACAATGCGGGAAAGATTATGAGCCTGACTAAAACC |
| | GCCCCCGACTACCTGGTGGGCCAGCAGCCCGTGGAGGACATTTCCAGC |
| | AATCGGATTTATAAAATTTTGGAACTAAACGGGTACGATCCCCAATATGC |
| | GGCTTCCGTCTTTCTGGGATGGGCCACGAAAAAGTTCGGCAAGAGGAA |
| | CACCATCTGGCTGTTTGGGCCTGCAACTACCGGGAAGACCAACATCGCG |
| | GAGGCCATAGCCCACACTGTGCCCTTCTACGGGTGCGTAAACTGGACCA |
| | ATGAGAACTTTCCCTTCAACGACTGTGTCGACAAGATGGTGATCTGGTG |
| | GGAGGAGGGGAAGATGACCGCCAAGGTCGTGGAGTCGGCCAAAGCCA |
| | TTCTCGGAGGAAGCAAGGTGCGCGTGGACCAGAAATGCAAGTCCTCGG |
| | CCCAGATAGACCCGACTCCCGTGATCGTCACCTCCAACACCAACATGTG |
| | CGCCGTGATTGACGGGAACTCAACGACCTTCGAACACCAGCAGCCGTT |
| | GCAAGACCGGATGTTCAAATTTGAACTCACCCGCCGTCTGGATCATGAC |
| | TTTGGGAAGGTCACCAAGCAGGAAGTCAAAGACTTTTTCCGGTGGGCA |
| | AAGGATCACGTGGTTGAGGTGGAGCATGAATTCTACGTCAAAAAGGGT |
| | GGAGCCAAGAAAAGACCCGCCCCCAGTGACGCAGATATAAGTGAGCCC |
| | AAACGGGTGCGCGAGTCAGTTGCGCAGCCATCGACGTCAGACGCGGAA |
| | GCTTCGATCAACTACGCAGACAGGTACCAAAACAAATGTTCTCGTCACG |
| | TGGGCATGAATCTGATGCTGTTTCCCTGCAGACAATGCGAGAGAATGAA |
| | TCAGAATTCAAATATCTGCTTCACTCACGGACAGAAAGACTGTTTAGAG |
| | TGCTTTCCCGTGTCAGAATCTCAACCCGTTTCTGTCGTCAAAAAGGCGT |
| | ATCAGAAACTGTGCTACATTCATCATATCATGGGAAAGGTGCCAGACGC |
| | TTGCACTGCCTGCGATCTGGTCAATGTGGATTTGGATGACTGCATCTTTG |
| | AACAATAA |
| MCP-Rep2-Y156F | MPGSSGSSASNFTQFVLVDNGGTGDVTVAPSNFANGVAEWISSNSRSQAYK |
| amino acid sequence | VTCSVRQSSAQNRKYTIKVEVPKVATQTVGGVELPVAAWRSYLNMELTIPIF |
| (MP-linker-MCP- | ATNSDCELIVKAMQGLLKDGNPIPSAIAANSGIYGSSGSSGFYEIVIKVPSDL |
| linker-remaining | DEHLPGISDSFVNWVAEKEWELPPDSDMDLNLIEQAPLTVAEKLQRDFLTE |
| Rep2) | WRRVSKAPEALFFVQFEKGESYFHMHVLVETTGVKSMVLGRFLSQIREKLI |
| SEQ ID NO: 65 | QRIYRGIEPTLPNWFAVTKTRNGAGGGNKVVDECYIPNFLLPKTQPELQW |
| | AWTNMEQYLSACLNLTERKRLVAQHLTHVSQTQEQNKENQNPNSDAPVIR |
| | SKTSARYMELVGWLVDKGITSEKQWIQEDQASYISFNAASNSRSQIKAALD |
| | NAGKIMSLIKTAPDYLVGQQPVEDISSNRIYKILELNGYDPQYAASVFLGW |
| | ATKKFGKRNTIWLFGPATTGKTNIAEAIAHTVPFYGCVNWTNENFPFNDCV |
| | DKMVIWWEEGKMTAKVVESAKAILGGSKVRVDQKCKSSAQIDPTPVIVTS |
| | NTNMCAVIDGNSTTFEHQQPLQDRMFKFELTRRLDHDFGKVTKQEVKDFF |
| | RWAKDHVVEVEHEFYVKKGGAKKRPAPSDADISEPKRVRESVAQPSTSDA |
| | EASINYADRYQNKCSRHVGMNLMLFPCRQCERMNQNSNICFTHGQKDCL |
| | ECFPVSESQPVSVVKKAYQKLCYIHHIMGKVPDACTACDLVNVDLDDCIFE |
| | Q |
| MCP-Rep2-KDE-mu | ATGCCcGGCAGCTCCGGCAGTAGCgcttctaactttactcagttcgttctcgtcgacaatggcgg |
| nucleic acid | aactggcgacgtgactgtcgccccaagcaacttcgctaacggggtcgctgaatggatcagctctaactcg |
| sequence | cgttcacaggcttacaaagtaacctgtagcgttcgtcagagctctgcgcagaatcgcaaatacaccatca |
| (MP-linker-MCP- | aagtcgaggtgcctaaagtggcaacccagactgttggtggagtagagcttcctgtagccgcatggcgttc |
| linker-remaining | gtacttaaatatggaactaaccattccaattttcgctacgaattccgactgcgagcttattgttaaggca |
| Rep2) | atgcaaggtctcctaaaagatggaaacccgattccctcagcaatcgcagcaaactccggcatctac |
| SEQ ID NO: 66 | GGCAGTAGTGGGTCCTCTGGGTTTTACGAGATTGTGATTAAGGTCCCCAGCGACCTTGACGAGCATCTGCC |
| | CGGCATTTCTGACAGCTTTGTGAACTGGGTGGCCGAGAAGGAATGGGA |
| | GTTGCCGCCAGATTCTGACATGGATCTGAATCTGATTGAGCAGGCACCC |
| | CTGACCGTGGCCGAGAAGCTGCAGCGCGACTTTCTGACGGAATGGCGC |
| | CGTGTGAGTAAGGCCCCGGAGGCCCTTTTCTTTGTGCAATTTGAGAAGG |
| | GAGAGAGCTACTTCCACATGCACGTGCTCGTGGAAACCACCGGGGTGA |
| | AATCCATGGTTTTGGGACGTTTCCTGAGTCAGATTCGCGAAAAACTGAT |
| | TCAGAGAATTTACCGCGGGATCGAGCCGACTTTGCCAAACTGGTTCGCG |
| | GTCACAAAGACCAGAAATGGCGCCGGAGGCGGGAACgcGGTGGTG |
| | GcT |
| | GcGTGCTACATCCCCAATTaCTTGCTCCCCAAAACCCAGCCTGAGCTCCA |
| | GTGGGCGTGGACTAATATGGAACAGTATTTAAGCGCCTGTTTGAATCTCA |
| | CGGAGCGTAAACGGTTGGTGGCGCAGCATCTGACGCACGTGTCGCAGA |
| | CGCAGGAGCAGAACAAAGAGAATCAGAATCCCAATTCTGATGCGCCGG |
| | TGATCAGATCAAAAACTTCAGCCAGGTACATGGAGCTGGTCGGGTGGCT |
| | CGTGGACAAGGGGATTACCTCGGAGAAGCAGTGGATCCAGGAGGACCA |
| | GGCCTCATACATCTCCTTCAATGCGGCCTCCAACTCGCGGTCCCAAATCA |
| | AGGCTGCCTTGGACAATGCGGGAAAGATTATGAGCCTGACTAAAACCGC |
| | CCCCGACTACCTGGTGGGCCAGCAGCCCGTGGAGGACATTTCCAGCAAT |
| | CGGATTTATAAAATTTTGGAACTAAACGGGTACGATCCCCAATATGCGGC |
| | TTCCGTCTTTCTGGGATGGGCCACGAAAAAGTTCGGCAAGAGGAACAC |
| | CATCTGGCTGTTTGGGCCTGCAACTACCGGGAAGACCAACATCGCGGA |
| | GGCCATAGCCCACACTGTGCCCTTCTACGGGTGCGTAAACTGGACCAAT |
| | GAGAACTTTCCCTTCAACGACTGTGTCGACAAGATGGTGATCTGGTGGG |
| | AGGAGGGGAAGATGACCGCCAAGGTCGTGGAGTCGGCCAAAGCCATTC |
| | TCGGAGGAAGCAAGGTGCGCGTGGACCAGAAATGCAAGTCCTCGGCCC |
| | AGATAGACCCGACTCCCGTGATCGTCACCTCCAACACCAACATGTGCGC |
| | CGTGATTGACGGGAACTCAACGACCTTCGAACACCAGCAGCCGTTGCA |
| | AGACCGGATGTTCAAATTTGAACTCACCCGCCGTCTGGATCATGACTTT |
| | GGGAAGGTCACCAAGCAGGAAGTCAAAGACTTTTTCCGGTGGGCAAAG |
| | GATCACGTGGTTGAGGTGGAGCATGAATTCTACGTCAAAAAGGGTGGA |
| | GCCAAGAAAAGACCCGCCCCCAGTGACGCAGATATAAGTGAGCCCAAA |
| | CGGGTGCGCGAGTCAGTTGCGCAGCCATCGACGTCAGACGCGGAAGCT |
| | TCGATCAACTACGCAGACAGGTACCAAAACAAATGTTCTCGTCACGTGG |
| | GCATGAATCTGATGCTGTTTCCCTGCAGACAATGCGAGAGAATGAATCA |
| | GAATTCAAATATCTGCTTCACTCACGGACAGAAAGACTGTTTAGAGTGC |
| | TTTCCCGTGTCAGAATCTCAACCCGTTTCTGTCGTCAAAAAGGCGTATC |
| | AGAAACTGTGCTACATTCATCATATCATGGGAAAGGTGCCAGACGCTTG |
| | CACTGCCTGCGATCTGGTCAATGTGGATTTGGATGACTGCATCTTTGAAC |
| | AATAA |
| MCP-Rep2-KDE-mu | MPGSSGSSASNFTQFVLVDNGGTGDVTVAPSNFANGVAEWISSNSRSQAYK |
| amino acid | VTCSVRQSSAQNRKYTIKVEVPKVATQTVGGVELPVAAWRSYLNMELTIPIF |
| sequence (MP- | ATNSDCELIVKAMQGLLKDGNPIPSAIAANSGIYGSSGSSGFYEIVIKVPSDL |
| linker-MCP-linker- | DEHLPGISDSFVNWVAEKEWELPPDSDMDLNLIEQAPLTVAEKLQRDFLTE |
| remaining Rep2) | WRRVSKAPEALFFVQFEKGESYFHMHVLVETTGVKSMVLGRFLSQIREKLI |
| SEQ ID NO: 67 | QRIYRGIEPTLPNWFAVTKTRNGAGGGNAVV |
| | AACYIPNYLLPKTQPELQW |
| | AWTNMEQYLSACLNLTERKRLVAQHLTHVSQTQEQNKENQNPNSDAPVIR |
| | SKTSARYMELVGWLVDKGITSEKQWIQEDQASYISFNAASNSRSQIKAALD |
| | NAGKIMSLTKTAPDYLVGQQPVEDISSNRIYKILELNGYDPQYAASVFLGW |
| | ATKKFGKRNTIWLFGPATTGKTNIAEAIAHTVPFYGCVNWTNENFPFNDCV |
| | DKMVIWWEEGKMTAKVVESAKAILGGSKVRVDQKCKSSAQIDPTPVIVTS |
| | NTNMCAVIDGNSTTFEHQQPLQDRMFKFELTRRLDHDFGKVTKQEVKDFF |
| | RWAKDHVVEVEHEFYVKKGGAKKRPAPSDADISEPKRVRESVAQPSTSDA |
| | EASINYADRYQNKCSRHVGMNLMLFPCRQCERMNQNSNICFTHGQKDCL |
| | ECFPVSESQPVSVVKKAYQKLCYIHHIMGKVPDACTACDLVNVDLDDCIFE |
| | Q |
| MCP-Rep2-EKE-mu | ATGCCcGGCAGCTCCGGCAGTAGCgcttctaactttactcagttcgttctcgtcgacaatggcgg |
| nucleic acid | aactggcgacgtgactgtcgccccaagcaacttcgctaacggggtcgctgaatggatcagctctaactcgc |
| sequence | gttcacaggcttacaaagtaacctgtagcgttcgtcagagctctgcgcagaatcgcaaatacaccatcaaa |
| (MP-linker-MCP- | gtcgaggtgcctaaagtggcaacccagactgttggtggagtagagcttcctgtagccgcatggcgttcgta |
| linker-remaining | cttaaatatggaactaaccattccaattttcgctacgaattccgactgcgagcttattgttaaggcaatgc |
| Rep2) | aaggtctcctaaaagatggaaacccgattccctcagcaatcgcagcaaactccggcatctacGGCAGTAGT |
| SEQ ID NO: 68 | GGGTCCTCTGGGTTTTACGAGATTGTGATTAAGGTCCCCAGCGACCTTGACGAGCATCTGCC |
| | CGGCATTTCTGACAGCTTTGTGAACTGGGTGGCCGAGAAGGAATGGGA |
| | GTTGCCGCCAGATTCTGACATGGATCTGAATCTGATTGAGCAGGCACCC |
| | CTGACCGTGGCCGAGAAGCTGCAGCGCGACTTTCTGACGGAATGGCGC |
| | CGTGTGAGTAAGGCCCCGGAGGCCCTTTTCTTTGTGCAATTTGcGgcGGG |
| | AGcGAGCTACTTCCACATGCACGTGCTCGTGGAAACCACCGGGGTGAA |
| | ATCCATGGTTTTGGGACGTTTCCTGAGTCAGATTCGCGAAAAACTGATT |
| | CAGAGAATTTACCGCGGGATCGAGCCGACTTTGCCAAACTGGTTCGCGG |
| | TCACAAAGACCAGAAATGGCGCCGGAGGCGGGAACaaGGTGGTGGaTG |
| | AGTGCTACATCCCCAATTaCTTGCTCCCCAAAACCCAGCCTGAGCTCCAG |
| | TGGGCGTGGACTAATATGGAACAGTATTTAAGCGCCTGTTTGAATCTCAC |
| | GGAGCGTAAACGGTTGGTGGCGCAGCATCTGACGCACGTGTCGCAGAC |
| | GCAGGAGCAGAACAAAGAGAATCAGAATCCCAATTCTGATGCGCCGGT |
| | GATCAGATCAAAAACTTCAGCCAGGTACATGGAGCTGGTCGGGTGGCTC |
| | GTGGACAAGGGGATTACCTCGGAGAAGCAGTGGATCCAGGAGGACCAG |
| | GCCTCATACATCTCCTTCAATGCGGCCTCCAACTCGCGGTCCCAAATCAA |
| | GGCTGCCTTGGACAATGCGGGAAAGATTATGAGCCTGACTAAAACCGCC |
| | CCCGACTACCTGGTGGGCCAGCAGCCCGTGGAGGACATTTCCAGCAATC |
| | GGATTTATAAAATTTTGGAACTAAACGGGTACGATCCCCAATATGCGGCT |
| | TCCGTCTTTCTGGGATGGGCCACGAAAAAGTTCGGCAAGAGGAACACC |
| | ATCTGGCTGTTTGGGCCTGCAACTACCGGGAAGACCAACATCGCGGAG |
| | GCCATAGCCCACACTGTGCCCTTCTACGGGTGCGTAAACTGGACCAATG |
| | AGAACTTTCCCTTCAACGACTGTGTCGACAAGATGGTGATCTGGTGGGA |
| | GGAGGGGAAGATGACCGCCAAGGTCGTGGAGTCGGCCAAAGCCATTCT |
| | CGGAGGAAGCAAGGTGCGCGTGGACCAGAAATGCAAGTCCTCGGCCCA |
| | GATAGACCCGACTCCCGTGATCGTCACCTCCAACACCAACATGTGCGCC |
| | GTGATTGACGGGAACTCAACGACCTTCGAACACCAGCAGCCGTTGCAA |
| | GACCGGATGTTCAAATTTGAACTCACCCGCCGTCTGGATCATGACTTTG |
| | GGAAGGTCACCAAGCAGGAAGTCAAAGACTTTTTCCGGTGGGCAAAGG |
| | ATCACGTGGTTGAGGTGGAGCATGAATTCTACGTCAAAAAGGGTGGAG |
| | CCAAGAAAAGACCCGCCCCCAGTGACGCAGATATAAGTGAGCCCAAAC |
| | GGGTGCGCGAGTCAGTTGCGCAGCCATCGACGTCAGACGCGGAAGCTT |
| | CGATCAACTACGCAGACAGGTACCAAAACAAATGTTCTCGTCACGTGGG |
| | CATGAATCTGATGCTGTTTCCCTGCAGACAATGCGAGAGAATGAATCAG |
| | AATTCAAATATCTGCTTCACTCACGGACAGAAAGACTGTTTAGAGTGCT |
| | TTCCCGTGTCAGAATCTCAACCCGTTTCTGTCGTCAAAAAGGCGTATCA |
| | GAAACTGTGCTACATTCATCATATCATGGGAAAGGTGCCAGACGCTTGC |
| | ACTGCCTGCGATCTGGTCAATGTGGATTTGGATGACTGCATCTTTGAACA |
| | ATAA |
| MCP-Rep2-EKE-mu | MPGSSGSSASNFTQFVLVDNGGTGDVTVAPSNFANGVAEWISSNSRSQAYK |
| amino acid sequence | VTCSVRQSSAQNRKYTIKVEVPKVATQTVGGVELPVAAWRSYLNMELTIPIF |
| (MP-linker-MCP- | ATNSDCELIVKAMQGLLKDGNPIPSAIAANSGIYGSSGSSGFYEIVIKVPSDL |
| linker-remaining | DEHLPGISDSFVNWVAEKEWELPPDSDMDLNLIEQAPLTVAEKLQRDFLTE |
| Rep2) | WRRVSKAPEALFFVQFAAG |
| SEQ ID NO: 69 | ASYFHMHVLVETTGVKSMVLGRFLSQIREKLI |
| | QRIYRGIEPTLPNWFAVTKTRNGAGGGNKVVDECYIPNYLLPKTQPELQW |
| | AWTNMEQYLSACLNLTERKRLVAQHLTHVSQTQEQNKENQNPNSDAPVIR |
| | SKTSARYMELVGWLVDKGITSEKQWIQEDQASYISFNAASNSRSQIKAALD |
| | NAGKIMSLTKTAPDYLVGQQPVEDISSNRIYKILELNGYDPQYAASVFLGW |
| | ATKKFGKRNTIWLFGPATTGKTNIAEAIAHTVPFYGCVNWTNENFPFNDCV |
| | DKMVIWWEEGKMTAKVVESAKAILGGSKVRVDQKCKSSAQIDPTPVIVTS |
| | NTNMCAVIDGNSTTFEHQQPLQDRMFKFELTRRLDHDFGKVTKQEVKDFF |
| | RWAKDHVVEVEHEFYVKKGGAKKRPAPSDADISEPKRVRESVAQPSTSDA |
| | EASINYADRYQNKCSRHVGMNLMLFPCRQCERMNQNSNICFTHGQKDCL |
| | ECFPVSESQPVSVVKKAYQKLCYIHHIMGKVPDACTACDLVNVDLDDCIFE |
| | Q |
| 2×MCP-Rep2 nucleic | ATGCCcGGCAGCTCCGGCAGTAGCGCTTCTAACTTTACTCAGTTCGTTCT |
| acid sequence (MP- | CGTCGACAATGGCGGAACTGGCGACGTGACTGTCGCCCCAAGCAACTTC |
| linker-2xMCP- | GCTAACGGGATCGCTGAATGGATCAGCTCTAACTCGCGTTCACAGGCTTA |
| linker- | CAAAGTAACCTGTAGCGTTCGTCAGAGCTCTGCGCAGAATCGCAAATACA |
| remaining Rep2) | CCATCAAAGTCGAGGTGCCTAAAGGCGCCTGGCGTTCGTACTTAAATATG |
| SEQ ID NO: 70 | GAACTAACCATTCCAATTTTCGCCACGAATTCCGACTGCGAGCTTATTGTT |
| | AAGGCAATGCAAGGTCTCCTAAAAGATGGAAACCCGATTCCCTCAGCAAT |
| | CGCAGCAAACTCCGGCATCTACGGTGGTGGAGGAGGAATGGCGTCCAAT |
| | TTCACGCAGTTCGTCCTGGTTGACAACGGGGGGACTGGGGACGTTACGG |
| | TCGCTCCGAGCAACTTTGCCAATGGTATTGCGGAGTGGATTTCTTCTAAT |
| | TCACGGTCCCAAGCTTACAAAGTGACCTGTTCCGTGCGGCAAAGTTCTGC |
| | TCAGAATAGAAAGTACACTATAAAGGTCGAAGTCCCTAAGGGGGCCTGGC |
| | GATCATATCTCAATATGGAGCTTACCATCCCAATATTTGCCACTAATTCTGA |
| | TTGTGAATTGATTGTCAAAGCAATGCAAGGACTCTTGAAAGACGGAAACC |
| | CAATCCCCAGCGCAATCGCAGCCAACTCCGGTATATACGGCAGTAGTGGG |
| | TCCTCTGGGTTTTACGAGATTGTGATTAAGGTCCCCAGCGACCTTGACG |
| | AGCATCTGCCCGGCATTTCTGACAGCTTTGTGAACTGGGTGGCCGAGAA |
| | GGAATGGGAGTTGCCGCCAGATTCTGACATGGATCTGAATCTGATTGAG |
| | CAGGCACCCCTGACCGTGGCCGAGAAGCTGCAGCGCGACTTTCTGACG |
| | GAATGGCGCCGTGTGAGTAAGGCCCCGGAGGCCCTTTTCTTTGTGCAAT |
| | TTGAGAAGGGAGAGAGCTACTTCCACATGCACGTGCTCGTGGAAACCA |
| | CCGGGGTGAAATCCATGGTTTTGGGACGTTTCCTGAGTCAGATTCGCGA |
| | AAAACTGATTCAGAGAATTTACCGCGGGATCGAGCCGACTTTGCCAAAC |
| | TGGTTCGCGGTCACAAAGACCAGAAATGGCGCCGGAGGCGGGAACAA |
| | GGTGGTGGATGAGTGCTACATCCCCAATTACTTGCTCCCCAAAACCCAG |
| | CCTGAGCTCCAGTGGGCGTGGACTAATATGGAACAGTATTTAAGCGCCT |
| | GTTTGAATCTCACGGAGCGTAAACGGTTGGTGGCGCAGCATCTGACGCA |
| | CGTGTCGCAGACGCAGGAGCAGAACAAAGAGAATCAGAATCCCAATTC |
| | TGATGCGCCGGTGATCAGATCAAAAACTTCAGCCAGGTACATGGAGCTG |
| | GTCGGGTGGCTCGTGGACAAGGGGATTACCTCGGAGAAGCAGTGGATC |
| | CAGGAGGACCAGGCCTCATACATCTCCTTCAATGCGGCCTCCAACTCGC |
| | GGTCCCAAATCAAGGCTGCCTTGGACAATGCGGGAAAGATTATGAGCCT |
| | GACTAAAACCGCCCCCGACTACCTGGTGGGCCAGCAGCCCGTGGAGGA |
| | CATTTCCAGCAATCGGATTTATAAAATTTTGGAACTAAACGGGTACGATC |
| | CCCAATATGCGGCTTCCGTCTTTCTGGGATGGGCCACGAAAAAGTTCGG |
| | CAAGAGGAACACCATCTGGCTGTTTGGGCCTGCAACTACCGGGAAGAC |
| | CAACATCGCGGAGGCCATAGCCCACACTGTGCCCTTCTACGGGTGCGTA |
| | AACTGGACCAATGAGAACTTTCCCTTCAACGACTGTGTCGACAAGATGG |
| | TGATCTGGTGGGAGGAGGGGAAGATGACCGCCAAGGTCGTGGAGTCGG |
| | CCAAAGCCATTCTCGGAGGAAGCAAGGTGCGCGTGGACCAGAAATGCA |
| | AGTCCTCGGCCCAGATAGACCCGACTCCCGTGATCGTCACCTCCAACAC |
| | CAACATGTGCGCCGTGATTGACGGGAACTCAACGACCTTCGAACACCA |
| | GCAGCCGTTGCAAGACCGGATGTTCAAATTTGAACTCACCCGCCGTCTG |
| | GATCATGACTTTGGGAAGGTCACCAAGCAGGAAGTCAAAGACTTTTTCC |
| | GGTGGGCAAAGGATCACGTGGTTGAGGTGGAGCATGAATTCTACGTCA |
| | AAAAGGGTGGAGCCAAGAAAAGACCCGCCCCCAGTGACGCAGATATAA |
| | GTGAGCCCAAACGGGTGCGCGAGTCAGTTGCGCAGCCATCGACGTCAG |
| | ACGCGGAAGCTTCGATCAACTACGCAGACAGGTACCAAAACAAATGTT |
| | CTCGTCACGTGGGCATGAATCTGATGCTGTTTCCCTGCAGACAATGCGA |
| | GAGAATGAATCAGAATTCAAATATCTGCTTCACTCACGGACAGAAAGAC |
| | TGTTTAGAGTGCTTTCCCGTGTCAGAATCTCAACCCGTTTCTGTCGTCAA |
| | AAAGGCGTATCAGAAACTGTGCTACATTCATCATATCATGGGAAAGGTG |
| | CCAGACGCTTGCACTGCCTGCGATCTGGTCAATGTGGATTTGGATGACT |
| | GCATCTTTGAACAATAA |
| 2×MCP-Rep2 amino | MPGSSGSSASNFTQFVLVDNGGTGDVTVAPSNFANGIAEWISSNSRSQAYK |
| acid sequence (MP- | VTCSVRQSSAQNRKYTIKVEVPKGAWRSYLNMELTIPIFATNSDCELIVKAM |
| linker-2×MCP- | QGLLKDGNPIPSAIAANSGIYGGGGGMASNFTQFVLVDNGGTGDVTVAPS |
| linker- | NFANGIAEWISSNSRSQAYKVTCSVRQSSAQNRKYTIKVEVPKGAWRSYLN |
| remaining Rep2) | MELTIPIFATNSDCELIVKAMQGLLKDGNPIPSAIAANSGIYGSSGSSGFYEI |
| SEQ ID NO: 71 | VIKVPSDLDEHLPGISDSFVNWVAEKEWELPPDSDMDLNLIEQAPLTVAEK |
| | LQRDFLTEWRRVSKAPEALFFVQFEKGESYFHMHVLVETTGVKSMVLGRF |
| | LSQIREKLIQRIYRGIEPTLPNWFAVTKTRNGAGGGNKVVDECYIPNYLLPK |
| | TQPELQWAWTNMEQYLSACLNLTERKRLVAQHLTHVSQTQEQNKENQNP |
| | NSDAPVIRSKTSARYMELVGWLVDKGITSEKQWIQEDQASYISFNAASNSR |
| | SQIKAALDNAGKIMSLTKTAPDYLVGQQPVEDISSNRIYKILELNGYDPQYA |
| | ASVFLGWATKKFGKRNTIWLFGPATTGKTNIAEAIAHTVPFYGCVNWTNE |
| | NFPFNDCVDKMVIWWEEGKMTAKVVESAKAILGGSKVRVDQKCKSSAQI |
| | DPTPVIVTSNTNMCAVIDGNSTTFEHQQPLQDRMFKFELTRRLDHDFGKVT |
| | KQEVKDFFRWAKDHVVEVEHEFYVKKGGAKKRPAPSDADISEPKRVRESV |
| | AQPSTSDAEASINYADRYQNKCSRHVGMNLMLFPCRQCERMNQNSNICFT |
| | HGQKDCLECFPVSESQPVSVVKKAYQKLCYIHHIMGKVPDACTACDLVNV |
| | DLDDCIFEQ |
| PCP-Rep2-Y156F | ATGCCcGGCAGCTCCGGCAGTAGCtccaaaacaatagtcctctccgtaggggaggcaacacg |
| nucleic acid | gactttgaccgaaatccagtcaaccgctgaccgacaaatctttgaagagaaagtagggcctcttgtgggcc |
| sequence | gactgcgcttgactgcaagcttgcgacaaaacggcgcaaagactgcctatagggtcaaccttaaactcgac |
| (MP-linker-PCP- | caagccgacgtggtcgatagcggtctccctaaggttcggtatacgcaggtctggagtcatgacgtaacaat |
| linker-remaining | cgtagcaaacagcacagaagcctcccgaaaaagcctctacgatctgacgaaatccttggtggctacgtcac |
| Rep2) | aggtggaagacctcgttgtcaaccttgtacctctgggtcgaGGCAGTAGTGGGTCCTCTGGGTTTTACGAG |
| SEQ ID NO: 72 | ATTGTGATTAAGGTCCCCAGCGACCTTGACGAGCATCTGCCCGGCATTTCTGA |
| | CAGCTTTGTGAACTGGGTGGCCGAGAAGGAATGGGAGTTGCCGCCAGA |
| | TTCTGACATGGATCTGAATCTGATTGAGCAGGCACCCCTGACCGTGGCC |
| | GAGAAGCTGCAGCGCGACTTTCTGACGGAATGGCGCCGTGTGAGTAAG |
| | GCCCCGGAGGCCCTTTTCTTTGTGCAATTTGAGAAGGGAGAGAGCTACT |
| | TCCACATGCACGTGCTCGTGGAAACCACCGGGGTGAAATCCATGGTTTT |
| | GGGACGTTTCCTGAGTCAGATTCGCGAAAAACTGATTCAGAGAATTTAC |
| | CGCGGGATCGAGCCGACTTTGCCAAACTGGTTCGCGGTCACAAAGACC |
| | AGAAATGGCGCCGGAGGCGGGAACAAGGTGGTGGATGAGTGCTACATC |
| | CCCAATTtCTTGCTCCCCAAAACCCAGCCTGAGCTCCAGTGGGCGTGGA |
| | CTAATATGGAACAGTATTTAAGCGCCTGTTTGAATCTCACGGAGCGTAAA |
| | CGGTTGGTGGCGCAGCATCTGACGCACGTGTCGCAGACGCAGGAGCAG |
| | AACAAAGAGAATCAGAATCCCAATTCTGATGCGCCGGTGATCAGATCAA |
| | AAACTTCAGCCAGGTACATGGAGCTGGTCGGGTGGCTCGTGGACAAGG |
| | GGATTACCTCGGAGAAGCAGTGGATCCAGGAGGACCAGGCCTCATACAT |
| | CTCCTTCAATGCGGCCTCCAACTCGCGGTCCCAAATCAAGGCTGCCTTG |
| | GACAATGCGGGAAAGATTATGAGCCTGACTAAAACCGCCCCCGACTACC |
| | TGGTGGGCCAGCAGCCCGTGGAGGACATTTCCAGCAATCGGATTTATAA |
| | AATTTTGGAACTAAACGGGTACGATCCCCAATATGCGGCTTCCGTCTTTC |
| | TGGGATGGGCCACGAAAAAGTTCGGCAAGAGGAACACCATCTGGCTGT |
| | TTGGGCCTGCAACTACCGGGAAGACCAACATCGCGGAGGCCATAGCCC |
| | ACACTGTGCCCTTCTACGGGTGCGTAAACTGGACCAATGAGAACTTTCC |
| | CTTCAACGACTGTGTCGACAAGATGGTGATCTGGTGGGAGGAGGGGAA |
| | GATGACCGCCAAGGTCGTGGAGTCGGCCAAAGCCATTCTCGGAGGAAG |
| | CAAGGTGCGCGTGGACCAGAAATGCAAGTCCTCGGCCCAGATAGACCC |
| | GACTCCCGTGATCGTCACCTCCAACACCAACATGTGCGCCGTGATTGAC |
| | GGGAACTCAACGACCTTCGAACACCAGCAGCCGTTGCAAGACCGGATG |
| | TTCAAATTTGAACTCACCCGCCGTCTGGATCATGACTTTGGGAAGGTCA |
| | CCAAGCAGGAAGTCAAAGACTTTTTCCGGTGGGCAAAGGATCACGTGG |
| | TTGAGGTGGAGCATGAATTCTACGTCAAAAAGGGTGGAGCCAAGAAAA |
| | GACCCGCCCCCAGTGACGCAGATATAAGTGAGCCCAAACGGGTGCGCG |
| | AGTCAGTTGCGCAGCCATCGACGTCAGACGCGGAAGCTTCGATCAACT |
| | ACGCAGACAGGTACCAAAACAAATGTTCTCGTCACGTGGGCATGAATCT |
| | GATGCTGTTTCCCTGCAGACAATGCGAGAGAATGAATCAGAATTCAAAT |
| | ATCTGCTTCACTCACGGACAGAAAGACTGTTTAGAGTGCTTTCCCGTGT |
| | CAGAATCTCAACCCGTTTCTGTCGTCAAAAAGGCGTATCAGAAACTGTG |
| | CTACATTCATCATATCATGGGAAAGGTGCCAGACGCTTGCACTGCCTGCG |
| | ATCTGGTCAATGTGGATTTGGATGACTGCATCTTTGAACAATAA |
| PCP-Rep2-V156F | MPGSSGSSSKTIVLSVGEATRTLTEIQSTADRQIFEEKVGPLVGRLRLTASLR |
| amino acid | QNGAKTAYRVNLKLDQADVVDSGLPKVRYTQVWSHDVTIVANSTEASRKS |
| sequence (MP- | LYDLTKSLVATSQVEDLVVNLVPLGRGSSGSSGFYEIVIKVPSDLDEHLPGIS |
| linker-PCP- | DSFVNWVAEKEWELPPDSDMDLNLIEQAPLTVAEKLQRDFLTEWRRVSKA |
| linker-remaining | PEALFFVQFEKGESYFHMHVLVETTGVKSMVLGRFLSQIREKLIQRIYRGIE |
| Rep2) | PTLPNWFAVTKTRNGAGGGNKVVDECYIPNFLLPKTQPELQWAWINMEQ |
| SEQ ID NO: 73 | YLSACLNLTERKRLVAQHLTHVSQTQEQNKENQNPNSDAPVIRSKTSARY |
| | MELVGWLVDKGITSEKQWIQEDQASYISFNAASNSRSQIKAALDNAGKIM |
| | SLTKTAPDYLVGQQPVEDISSNRIYKILELNGYDPQYAASVFLGWATKKFG |
| | KRNTIWLFGPATTGKTNIAEAIAHTVPFYGCVNWTNENFPFNDCVDKMVI |
| | WWEEGKMTAKVVESAKAILGGSKVRVDQKCKSSAQIDPTPVIVTSNTNM |
| | CAVIDGNSTTFEHQQPLQDRMFKFELTRRLDHDFGKVTKQEVKDFFRWAK |
| | DHVVEVEHEFYVKKGGAKKRPAPSDADISEPKRVRESVAQPSTSDAEASIN |
| | YADRYQNKCSRHVGMNLMLFPCRQCERMNQNSNICFTHGQKDCLECFPV |
| | SESQPVSVVKKAYQKLCYIHHIMGKVPDACTACDLVNVDLDDCIFEQ |
| COM-2Rep-Y156F | ATGCCcGGCAGCTCCGGCAGTAGCatgaaatcaattcgctgtaaaaactgcaacaaactgttat |
| nucleic acid | ttaaggcggattcctttgatcacattgaaatcaggtgtccgcgttgcaaacgtcacatcataatgctgaat |
| sequence | gcctgcgagcatcccacggagaaacattgtgggaaaagagaaaaaatcacgcattctgacgaaaccgtgcg |
| (MP-linker-COM- | ttatGGCAGTAGTGGGTCCTCTGGGTTTTACGAGATTGTGATTAAGGTCCCCAGCG |
| linker-remaining | ACCTTGACGAGCATCTGCCCGGCATTTCTGACAGCTTTGTGAACTGGGT |
| Rep2) | GGCCGAGAAGGAATGGGAGTTGCCGCCAGATTCTGACATGGATCTGAAT |
| SEQ ID NO: 74 | CTGATTGAGCAGGCACCCCTGACCGTGGCCGAGAAGCTGCAGCGCGAC |
| | TTTCTGACGGAATGGCGCCGTGTGAGTAAGGCCCCGGAGGCCCTTTTCT |
| | TTGTGCAATTTGAGAAGGGAGAGAGCTACTTCCACATGCACGTGCTCGT |
| | GGAAACCACCGGGGTGAAATCCATGGTTTTGGGACGTTTCCTGAGTCAG |
| | ATTCGCGAAAAACTGATTCAGAGAATTTACCGCGGGATCGAGCCGACTT |
| | TGCCAAACTGGTTCGCGGTCACAAAGACCAGAAATGGCGCCGGAGGCG |
| | GGAACAAGGTGGTGGATGAGTGCTACATCCCCAATTtCTTGCTCCCCAA |
| | AACCCAGCCTGAGCTCCAGTGGGCGTGGACTAATATGGAACAGTATTTA |
| | AGCGCCTGTTTGAATCTCACGGAGCGTAAACGGTTGGTGGCGCAGCATC |
| | TGACGCACGTGTCGCAGACGCAGGAGCAGAACAAAGAGAATCAGAATC |
| | CCAATTCTGATGCGCCGGTGATCAGATCAAAAACTTCAGCCAGGTACAT |
| | GGAGCTGGTCGGGTGGCTCGTGGACAAGGGGATTACCTCGGAGAAGCA |
| | GTGGATCCAGGAGGACCAGGCCTCATACATCTCCTTCAATGCGGCCTCC |
| | AACTCGCGGTCCCAAATCAAGGCTGCCTTGGACAATGCGGGAAAGATT |
| | ATGAGCCTGACTAAAACCGCCCCCGACTACCTGGTGGGCCAGCAGCCC |
| | GTGGAGGACATTTCCAGCAATCGGATTTATAAAATTTTGGAACTAAACG |
| | GGTACGATCCCCAATATGCGGCTTCCGTCTTTCTGGGATGGGCCACGAA |
| | AAAGTTCGGCAAGAGGAACACCATCTGGCTGTTTGGGCCTGCAACTAC |
| | CGGGAAGACCAACATCGCGGAGGCCATAGCCCACACTGTGCCCTTCTAC |
| | GGGTGCGTAAACTGGACCAATGAGAACTTTCCCTTCAACGACTGTGTCG |
| | ACAAGATGGTGATCTGGTGGGAGGAGGGGAAGATGACCGCCAAGGTCG |
| | TGGAGTCGGCCAAAGCCATTCTCGGAGGAAGCAAGGTGCGCGTGGACC |
| | AGAAATGCAAGTCCTCGGCCCAGATAGACCCGACTCCCGTGATCGTCAC |
| | CTCCAACACCAACATGTGCGCCGTGATTGACGGGAACTCAACGACCTTC |
| | GAACACCAGCAGCCGTTGCAAGACCGGATGTTCAAATTTGAACTCACC |
| | CGCCGTCTGGATCATGACTTTGGGAAGGTCACCAAGCAGGAAGTCAAA |
| | GACTTTTTCCGGTGGGCAAAGGATCACGTGGTTGAGGTGGAGCATGAAT |
| | TCTACGTCAAAAAGGGTGGAGCCAAGAAAAGACCCGCCCCCAGTGACG |
| | CAGATATAAGTGAGCCCAAACGGGTGCGCGAGTCAGTTGCGCAGCCATC |
| | GACGTCAGACGCGGAAGCTTCGATCAACTACGCAGACAGGTACCAAAA |
| | CAAATGTTCTCGTCACGTGGGCATGAATCTGATGCTGTTTCCCTGCAGAC |
| | AATGCGAGAGAATGAATCAGAATTCAAATATCTGCTTCACTCACGGACA |
| | GAAAGACTGTTTAGAGTGCTTTCCCGTGTCAGAATCTCAACCCGTTTCT |
| | GTCGTCAAAAAGGCGTATCAGAAACTGTGCTACATTCATCATATCATGGG |
| | AAAGGTGCCAGACGCTTGCACTGCCTGCGATCTGGTCAATGTGGATTTG |
| | GATGACTGCATCTTTGAACAATAA |
| COM-Rep2-Y156F | MPGSSGSSMKSIRCKNCNKLLFKADSFDHIEIRCPRCKRHIIMLNACEHPT |
| amino acid sequence | EKHCGKREKITHSDETVRYGSSGSSGFYEIVIKVPSDLDEHLPGISDSFVNW |
| (MP-linker-COM- | VAEKEWELPPDSDMDLNLIEQAPLTVAEKLQRDFLTEWRRVSKAPEALFFV |
| linker-remaining | QFEKGESYFHMHVLVETTGVKSMVLGRFLSQIREKLIQRIYRGIEPTLPNW |
| Rep2) | FAVTKTRNGAGGGNKVVDECYIPNFLLPKTQPELQWAWTNMEQYLSACL |
| SEQ ID NO: 75 | NLTERKRLVAQHLTHVSQTQEQNKENQNPNSDAPVIRSKTSARYMELVGW |
| | LVDKGITSEKQWIQEDQASYISFNAASNSRSQIKAALDNAGKIMSLTKTAPD |
| | YLVGQQPVEDISSNRIYKILELNGYDPQYAASVFLGWATKKFGKRNTIWLF |
| | GPATTGKTNIAEAIAHTVPFYGCVNWTNENFPFNDCVDKMVIWWEEGKM |
| | TAKVVESAKAILGGSKVRVDQKCKSSAQIDPTPVIVTSNTNMCAVIDGNST |
| | TFEHQQPLQDRMFKFELTRRLDHDFGKVTKQEVKDFFRWAKDHVVEVEH |
| | EFYVKKGGAKKRPAPSDADISEPKRVRESVAQPSTSDAEASINYADRYQNK |
| | CSRHVGMNLMLFPCRQCERMNQNSNICFTHGQKDCLECFPVSESQPVSVV |
| | KKAYQKLCYIHHIMGKVPDACTACDLVNVDLDDCIFEQ |

*Sequence elements are matched based on formatting styles (e.g., double underline, bold, and/or italic fonts, etc.)

TABLE D
Nucleic Acid Sequence and Amino Acid Sequence of AAP and MCP fusion proteins

| AAP and MCP fusion | |
|---|---|
| proteins | Sequences |
| MCP-AAP(DJ) nucleic | atgGGCAGCTCCGGCAGTAGCgcttctaactttactcagttcgttctcgtcgacaatggcggaactgg |
| acid sequence | cgacgtgactgtcgccccaagcaacttcgctaacggggtcgctgaatggatcagctctaactcgcgttcacag |
| (linker- | gcttacaaagtaacctgtagcgttcgtcagagctctgcgcagaatcgcaaatacaccatcaaagtcgaggtgc |
| MCP-linker-AAP) | ctaaagtggcaacccagactgttggtggagtagagcttcctgtagccgcatggcgttcgtacttaaatatgga |
| SEQ ID NO: 76 | actaaccattccaattttcgctacgaattccgactgcgagcttattgttaaggcaatgcaaggtctcctaaaa |
| | gatggaaacccgattccctcagcaatcgcagcaaactccggcatctacGGCAGTAGTGGGTCCTCTctggaga |
| | cgcagactcagtcccagaccctcaaccaatcggagaacctcccgcagccccctcaggtgtgggatctcttaca |
| | atggctgcaggcggtggcgcaccaatggcagacaataacgagggcgccgacggagtgggtaattcctcgggaa |
| | attggcattgcgattccacatggatgggcgacagagtcatcaccaccagcacccgaacctgggccctgcccac |
| | ctacaacaaccacctctacaagcaaatctccaacagcacatctggaggatcttcaaatgacaacgcctacttc |
| | ggctacagcaccccctgggggtattttgactttaacagattccactgccacttttcaccacgtgactggcagc |
| | gactcatcaacaacaactggggattccggcccaagagactcagcttcaagctcttcaacatccaggtcaagga |
| | ggtcacgcagaatgaaggcaccaagaccatcgccaataacctcaccagcaccatccaggtgtttacggactcg |
| | gagtaccagctgccgtacgttctcggctctgcccaccagggctgcctgcctccgttcccggcggacgtgttca |
| | tga |
| MCP-AAP(DJ) amino | MGSSGSSASNFTQFVLVDNGGTGDVTVAPSNFANGVAEWISSNSRSQAYKV |
| acid sequence | TCSVRQSSAQNRKYTIKVEVPKVATQTVGGVELPVAAWRSYLNMELTIPIFA |
| (linker- | TNSDCELIVKAMQGLLKDGNPIPSALAANSGIYGSSGSSLETQTQSQTLNQS |
| MCP-linker-AAP) | ENLPQPPQVWDLLQWLQAVAHQWQTITRAPTEWVIPREIGIAIPHGWATES |
| SEQ ID NO: 77 | SPPAPEPGPCPPTTTTSTSKSPTAHLEDLQMTTPTSATAPPGGILTLTDSTATF |
| | HHVTGSDSSTTTGDSGPRDSASSSSTSRSRRSRRMKAPRPSPITSPAPSRCLR |
| | TRSTSCRTFSALPTRAACLRSRRTCS |
| AAP-MCP(DJ) nucleic | atgctggagacgcagactcagtcccagaccctcaaccaatcggagaacctcccgcagccccctcaggtgtggg |
| acid sequence (AAP- | atctcttacaatggctgcaggcggtggcgcaccaatggcagacaataacgagggcgccgacggagtgggtaat |
| linker-MCP-linker) | tcctcgggaaattggcattgcgattccacatggatgggcgacagagtcatcaccaccagcacccgaacctggg |
| SEQ ID NO: 78 | ccctgcccacctacaacaaccacctctacaagcaaatctccaacagcacatctggaggatcttcaaatgacaa |
| | cgcctacttcggctacagcaccccctgggggtattttgactttaacagattccactgccacttttcaccacgt |
| | gactggcagcgactcatcaacaacaactggggattccggcccaagagactcagcttcaagctcttcaacatcc |
| | aggtcaaggaggtcacgcagaatgaaggcaccaagaccatcgccaataacctcaccagcaccatccaggtgtt |
| | tacggactcggagtaccagctgccgtacgttctcggctctgcccaccagggctgcctgcctccgttcccggcg |
| | gacgtgttcaGGCAGCTCCGGCAGTAGCgcttctaactttactcagttcgttctcgtcgacaatggcggaact |
| | ggcgacgtgactgtcgccccaagcaacttcgctaacggggtcgctgaatggatcagctctaactcgcgttcac |
| | aggcttacaaagtaacctgtagcgttcgtcagagctctgcgcagaatcgcaaatacaccatcaaagtcgaggt |
| | gcctaaagtggcaacccagactgttggtggagtagagcttcctgtagccgcatggcgttcgtacttaaatatg |
| | gaactaaccattccaattttcgctacgaattccgactgcgagcttattgttaaggcaatgcaaggtctcctaa |
| | aagatggaaacccgattccctcagcaatcgcagcaaactccggcatctacGGCAGTAGTGGGTCCTCTtga |
| AAP-MCP(DJ) amino | MLETQTQSQTLNQSENLPQPPQVWDLLQWLQAVAHQWQTITRAPTEWVIP |
| acid sequence (AAP- | REIGIAIPHGWATESSPPAPEPGPCPPTTTTSTSKSPTAHLEDLQMTTPTSATA |
| linker-MCP-linker) | PPGGILTLTDSTATFHHVTGSDSSTTTGDSGPRDSASSSSTSRSRRSRRMKAP |
| SEQ ID NO: 79 | RPSPITSPAPSRCLRTRSTSCRTFSALPTRAACLRSRRTCSGSSGSSASNFTQF |
| | VLVDNGGTGDVTVAPSNFANGVAEWISSNSRSQAYKVTCSVRQSSAQNRKY |
| | TIKVEVPKVATQTVGGVELPVAAWRSYLNMELTIPIFATNSDCELIVKAMQG |
| | LLKDGNPIPSALAANSGIYGSSGSS |

*Sequence elements are matched based on formatting styles (e.g., double underline, bold and/or italic fonts, etc.)

TABLE E
Primer sequences

| Primer | Sequences |
|---|---|
| WPRE-F | CCCGTATGGCTTTCATTTTCTCC |
| SEQ ID NO: 80 | |
| WPRE-R | GGCAATGCCCCAACCAGTG |
| SEQ ID NO: 81 | |
| Cre-F | CCAGTAGATGCCACTAGCGA |
| SEQ ID NO: 82 | |
| Cre-R | GCCTGGAGATACAGCAGGTA |
| SEQ ID NO: 83 | |
| CAG-F | CTTCTCCTCCGGGCTGTAAT |
| SEQ ID NO: 84 | |
| CAG-R | CTTTCACGCAGCCACAGAAA |
| SEQ ID NO: 85 | |

* F and R stand for forward and reverse primers, respectively.

TABLE F
Stuffer sequence

| Stuffer | Sequences |
|---|---|
| Stuffer | atatttggagggcagcttgatttcgacttcgggagggaagctgcgccatgcgatgttatcggtgcggtgaatgcaaag |
| nucleic | aagataaccgcttccgaccaaatcaaccttactggaatcgatggtgtctccggtgtgaaagaacaccaacaggggtgt |
| acid | taccactaccgcaggaaaaggaggacgtgccgcgagacagcgacgaagtatcaccgacataatctgcgaaaactg |
| sequence | caaataccttccaacgaaacgcaccagaaataaacccaagccaatcccaaaagaatctgacgtaaaaaccttcaacta |
| SEQ ID NO: | cacggctcacctgtgggatatccggtggctaagacgtcgtgcgaggaaaacaaggccattgaccaaaatcgaagtta |
| 86 | cgaacaagaaagcgtcgagcgagctttaacgtgcgctaactgcggtcagaagctgcatgtgctggaagttcacgtgt |
| | gtgagcactgctgcgcagaactgatgagcgatccgaatagctcgatgcacgaggaagaaggccgccgctaaacca |
| | gcgcgaagacgatgtaaaaacgatgaatgccgggaatggtttcaccctgcattcgctaatcagtggtggtgctctcca |
| | gagtgtggaaccaagatagcactcgaacgacgaagtaaagaacgcgaaaaagcggaaaaagcagcagagaagaa |
| | acgacgacgagaggagcagaaacagaaagataaacttaagattcgaaaactcgccttaaagccccgcagttactgg |
| | attaaacaagcccaacaagccgtaaacgccttcatcagagaaagagaccgcgacttaccatgtatctcgtgcggaac |
| | gctcacgtctgctcagtgggatgccggacattaccggacaactgctgcggcacctcaactccgatttaatgaacgcaa |
| | tattcacaagcaatgcgtggtgtgcaaccagcacaaaagcggaaatctcgttccgtatcgcgtcgaactgattagccg |
| | catcgggcaggaagcagtagacgaaatcgaatcaaaccataaccgccatcgctggactatcgaagagtgcaaggcg |
| | atcaaggcagagtaccaacagaaactcaaagacctgcgaaatagcagaagtgaggccgcgccacgttctcagtaaa |
| | aaccattccagacatgctcgttgaagcatacggaaatcagacagaagtagcacgcagactgaaatgtagtcgcggta |
| | cggtcagaaaatacgttgatgataaagacgggaaaatgcacgccatcgtcaacgacgttctcatggttcatcgcggat |
| | ggagtgaaagaggcccgctattacgaaaaaattgatggcagcaaataccgaaatatttgggtagttggcgatctgcac |
| | ggatgctacacgaacctgatgaacaaactggatacgattggattcgacaacaaaaaagacctgcttatctcggtgggc |
| | gatttggttgatcgtggtgcagagaacgttgaatgcctggaattaatcacattcccctggttcagagctgtacgtggaaa |
| | ccatgagcaaatgatgattgatggcttatcagagcgtggaaacgttaatcactggctgcttaatggcggtggctggttct |
| | ttaatctcgattacgacaaagaaattctggctaaagctcttgcccataaagcagatgaacttccgttaatcatcgaactgg |
| | tgagcaaagataaaaaatatgttatctgccacgccgattatccctttgacgaatacgagtttggaaagccagttgatcatc |
| | agcaggtaatctggaaccgcgaacgaatcagcaactcacaaaacgggatcgtgaaagaaatcaaaggcgcggaca |
| | cgttcatctttggtcatacgccagcagtgaaaccactcaagtttgccaaccaaatgtatatcgataccggcgcagtgttct |
| | gcggaaacctaacattgattcaggtacagggagaaggcgcgccagactcgaaagcgtagctaaatttcattcgccaa |
| | aaagcccgatgatgagcgactcaccacgggccacggcttctgactctctttccggtactgatgtgatggctgctatggg |
| | gatggcgcaatcacaagccggattcggtatggctgcattctgcggtaagcacgaactcagccagaacgacaaacaa |
| | aaggctatcaactatctgatgcaatttgcacacaaggtatcggggaaataccgtggtgtggcaaagcttgaaggaaata |
| | ctaaggcaaaggtactgcaagtgctcgcaacattcgcttatgcggattattgccgtagtgccgcgacgccgggggca |
| | agatgcagagattgccatggtacaggccgtgcggttgatattgccaaaacagagctgtgggggagagttgtcgagaa |
| | agagtgcggaagatgcaaaggcgtcggctattcaaggatgccagcaagcgcagcatatcgcgctgtgacgatgcta |
| | atcccaaaccttacccaacccacctggtcacgcactgttaagccgctgtatgacgctctggtggtgcaatgccacaaa |
| | gaagagtcaatcgcagacaacattttgaatgcggtcacacgttagcagcatgattgccacggatggcaacatattaac |
| | ggcatgatattgacttattgaataaaattgggtaaatttgactcaacgatgggttaattcgctcgttgtggtagtgaggcc |
| | aaaagaggcggcgcttactaccgattccgcctagttggtcacttcgacgtatcgtctggaactccaaccatcgcaggca |
| | gagaggtctgcaaaatgcaatcccgaaacagttcgcaggtaatagttagagcctgcataacggtttcgggattttttatat |
| | ctgcacaacaggtaagagcattgagtcgataatcgtgaagagtcggcgagcctggttagccagtgctctttccgttgtg |
| | ctgaattaagcgaataccggaagcagaaccggatcaccaaatgcgtacaggcgtcatcgccgcccagcaacagcac |
| | aacccaaactgagccgtagccactgtctgtcctAaattcattagtaatagttacgctgcggccttttacacatgaccttcg |
| | tgaaagcgggtggcaggaggtcgcgctaacaacctcctgccgttttgcccgtgcatatcggtcacgaacaaatctgat |
| | tactaaacacagtagcctggatttgttctatcagtaatcgaccttattcctaattaaatagagcaaatcccctt |

SEQ ID NO: 88: Rep78 from AAV2, 621 aa
MPGFYEIVIKVPSDLDEHLPGISDSFVNWVAEKEWELPPDSDMDLNLIEQAPLTVAEKLQRDFLTEWRRVS
KAPEALFFVQFEKGESYFHMHVLVETTGVKSMVLGRFLSQIREKLIQRIYRGIEPTLPNWFAVTKTRNGAG

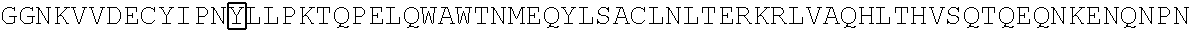

SDAPVIRSKTSARYMELVGWLVDKGITSEKQWIQEDQASYISFNAASNSRSQIKAALDNAGKIMSLTKTAP

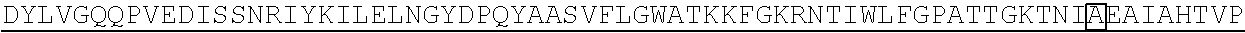

FYGCVNWTNENFPFNDCVDKMVIWWEEGKMTAKVVESAKAILGGSKVRVDQKCKSSAQIDPTPVIVTS

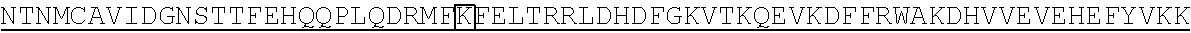

GGAKKRPAPSDADISEPKRVRESVAQPSTSDAEASINYADRYQNKCSRHVGMNLMLFPCRQCERMNQNS
NICFTHGQKDCLECFPVSESQPVSVVKKAYQKLCYIHHIMGKVPDACTACDLVNVDLDDCIFEQ
SEQ ID NO: 284: Rep68 from AAV2, 536 aa
MPGFYEIVIKVPSDLDEHLPGISDSFVNWVAEKEWELPPDSDMDLNLIEQAPLTVAEKLQRDFLTEWRRVS
KAPEALFFVQFEKGESYFHMHVLVETTGVKSMVLGRFLSQIREKLIQRIYRGIEPTLPNWFAVTKTRNGAG
GGNKVVDECYIPNYLLPKTQPELQWAWTNMEQYLSACLNLTERKRLVAQHLTHVSQTQEQNKENQNPN
SDAPVIRSKTSARYMELVGWLVDKGITSEKQWIQEDQASYISFNAASNSRSQIKAALDNAGKIMSLTKTAP
DYLVGQQPVEDISSNRIYKILELNGYDPQYAASVELGWATKKFGKRNTIWLFGPATTGKTNIAEAIAHTVP
FYGCVNWTNENFPFNDCVDKMVIWWEEGKMTAKVVESAKAILGGSKVRVDOKCKSSAQIDPTPVIVTS
NTNMCAVIDGNSTTFEHQQPLODRMEKFELTRRLDHDFGKVTKQEVKDFFRWAKDHVVEVEHEFYVKK
GGAKKRPAPSDADISEPKRVRESVAQPSTSDAEASINYADRLARGHSL
SEQ ID NO: 285: Rep52 from AAV2, 397 aa
MELVGWLVDKGITSEKOWIQEDQASYISFNAASNSRSQIKAALDNAGKIMSLIKTAPDYLVGQQPVEDISS
NRIYKILELNGYDPQYAASVELGWATKKFGKRNTIWLFGPATTGKTNIAEAIAHTVPFYGCVNWTNENFP
FNDCVDKMVIWWEEGKMTAKVVESAKAILGGSKVRVDQKCKSSAQIDPTPVIVTSNTNMCAVIDGNSTT
FEHQQPLQDRMFKFELTRRLDHDFGKVTKQEVKDFFRWAKDHVVEVEHEFYVKKGGAKKRPAPSDADI
SEPKRVRESVAQPSTSDAEASINYADRYQNKCSRHVGMNLMLFPCRQCERMNQNSNICFTHGQKDCLEC
FPVSESQPVSVVKKAYQKLCYIHHIMGKVPDACTACDLVNVDLDDCIFEQ
SEQ ID NO: 286: Rep40 from AAV2, 312 aa
MELVGWLVDKGITSEKQWIQEDQASYISFNAASNSRSQIKAALDNAGKIMSLTKTAPDYLVGQQPVEDISS
NRIYKILELNGYDPQYAASVELGWATKKFGKRNTIWLFGPATTGKTNIAEAIAHTVPFYGCVNWTNENFP
FNDCVDKMVIWWEEGKMTAKVVESAKAILGGSKVRVDQKCKSSAQIDPTPVIVTSNTNMCAVIDGNSTT
FEHQQPLQDRMFKFELTRRLDHDFGKVTKQEVKDFFRWAKDHVVEVEHEFYVKKGGAKKRPAPSDADI
SEPKRVRESVAQPSTSDAEASINYADRLARGHSL
SEQ ID NO: 186: Helicase domain shared by Rep78/68/52/40 from AAV2, position 308-463, 156 aa

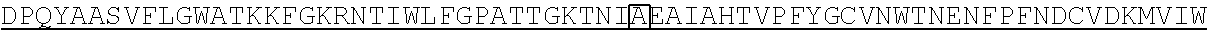

WEEGKMTAKVVESAKAILGGSKVRVDQKCKSSAQIDPTPVIVTSNTNMCAVIDGNSTTFEHQQPLQDRM

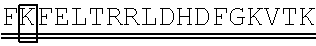

SEQ ID NO: 287: Helicase domain-A344V,K447F based on AAV2, 156 aa

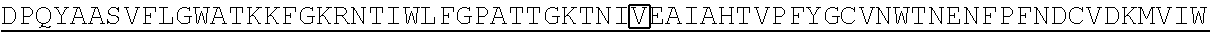

WEEGKMTAKVVESAKAILGGSKVRVDQKCKSSAQIDPTPVIVTSNTNMCAVIDGNSTTFEHQQPLQDRM

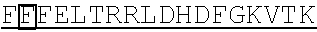

SEQ ID NO: 288: Rep78-A344V,K447F based on AAV2, 621 aa
MPGFYEIVIKVPSDLDEHLPGISDSFVNWVAEKEWELPPDSDMDLNLIEQAPLTVAEKLQRDFLTEWRRVS
KAPEALFFVQFEKGESYFHMHVLVETTGVKSMVLGRFLSQIREKLIQRIYRGIEPTLPNWFAVTKTRNGAG

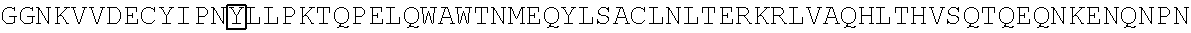

SDAPVIRSKTSARYMELVGWLVDKGITSEKQWIQEDQASYISFNAASNSRSQIKAALDNAGKIMSLTKTAP

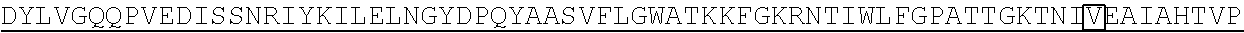

FYGCVNWTNENFPFNDCVDKMVIWWEEGKMTAKVVESAKAILGGSKVRVDQKCKSSAQIDPTPVIVTS

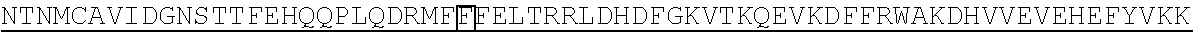

GGAKKRPAPSDADISEPKRVRESVAQPSTSDAEASINYADRYQNKCSRHVGMNLMLFPCRQCERMNQNS
NICFTHGQKDCLECFPVSESQPVSVVKKAYQKLCYIHHIMGKVPDACTACDLVNVDLDDCIFEQ
SEQ ID NO: 289: Rep78-Y156F,A344V,K447F based on AAV2, 621 aa
MPGFYEIVIKVPSDLDEHLPGISDSFVNWVAEKEWELPPDSDMDLNLIEQAPLTVAEKLQRDFLTEWRRVS
KAPEALFFVQFEKGESYFHMHVLVETTGVKSMVLGRFLSQIREKLIQRIYRGIEPTLPNWFAVTKTRNGAG

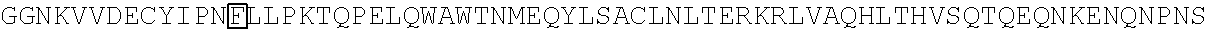

DAPVIRSKTSARYMELVGWLVDKGITSEKOWIQEDQASYISFNAASNSRSQIKAALDNAGKIMSLTKTAP

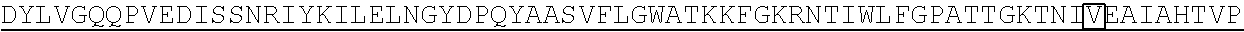

FYGCVNWTNENFPFNDCVDKMVIWWEEGKMTAKVVESAKAILGGSKVRVDQKCKSSAQIDPTPVIVTS

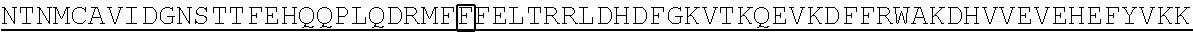

GGAKKRPAPSDADISEPKRVRESVAQPSTSDAEASINYADRYQNKCSRHVGMNLMLFPCRQCERMNQNS
NICFTHGQKDCLECFPVSESQPVSVVKKAYQKLCYIHHIMGKVPDACTACDLVNVDLDDCIFEQ
SEQ ID NO: 290, helicase domain-A346V,K449F based on AAV8, 156 aa

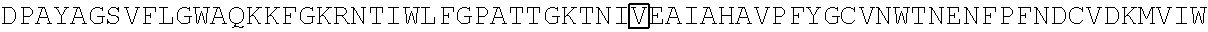

WEEGKMTAKVVESAKAILGGSKVRVDQKCKSSAQIDPTPVIVTSNTNMCAVIDGNSTTFEHQQPLQDRM

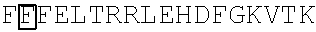

SEQ ID NO: 291, Rep78-A346V,K449F based on AAV8, 625 aa
MPGFYEIVIKVPSDLDEHLPGISDSFVNWVAEKEWELPPDSDMDRNLIEQAPLTVAEKLQRDFLVQWRRV
SKAPEALFFVQFEKGESYFHLHVLVETTGVKSMVLGRFLSQIREKLGPDHLPAGSSPTLPNWFAVTKDAV
MAPAGGNKVVDECYIPNYLLPKTQPELQWAWTNMEEYISACLNLAERKRLVAQHLTHVSQTQEQNKEN
LNPNSDAPVIRSKTSARYMELVGWLVDRGITSEKQWIQEDQASYISFNAASNSRSQIKAALDNAGKIMAL

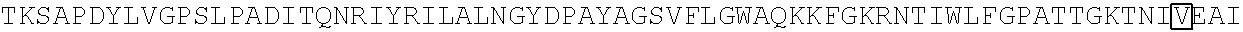

AHAVPFYGCVNWTNENFPFNDCVDKMVIWWEEGKMTAKVVESAKAILGGSKVRVDQKCKSSAQIDPTP

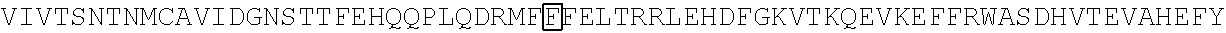

VRKGGASKRPAPDDADKSEPKRACPSVADPSTSDAEGAPVDFADRYQNKCSRHAGMLQMLFPCKTCER
MNQNFNICFTHGVRDCSECFPGVSESQPVVRKRTYRKLCAIHHLLGRAPEIACSACDLVNVDLDDCVSEQ

| | Full viral protein | Helicase domain |
|---|---|---|
| Abbreviations | sequences SEQ ID NO: | SEQ ID NO: |
| B19V (strain HV) | 89 | 187 |
| B19V | 90 | 188 |
| AAV10 | 91 | 189 |
| AAV11 | 92 | 190 |
| AAV7 | 93 | 191 |
| AAV8 | 94 | 192 |
| AAV9 | 95 | 193 |
| AAV1 | 96 | 194 |
| AAV6 | 97 | 195 |
| AAV12 | 98 | 196 |
| AAV2 | 88 | 186 |
| AAV13 | 99 | 197 |
| AAV4 | 100 | 198 |
| AAV3A | 101 | 199 |
| AAV3B | 102 | 200 |
| AAV5 | 103 | 201 |
| bPARV1 | 104 | 202 |
| HBoV1 | 105 | 203 |
| CPV-2 | 106 | 204 |
| HaPV | 107 | 205 |
| PPV | 108 | 206 |
| AMDV | 109 | 207 |
| SINV-3 | 110 | 208 |
| ABPV-R | 111 | 209 |
| CrPV | 112 | 210 |
| DCV | 113 | 211 |
| EoPV | 114 | 212 |
| BEC-NA1 | 115 | 213 |
| BEC-NB | 116 | 214 |
| CaCV | 117 | 215 |
| FCV | 118 | 216 |
| FCV-C | 119 | 217 |
| SMSV-1 | 120 | 218 |
| SMSV-4 | 121 | 219 |
| VESV | 122 | 220 |
| PES | 123 | 221 |
| SaV-GII | 124 | 222 |
| SV | 125 | 223 |
| SaV-GI | 126 | 224 |
| EBHSV-GD | 127 | 225 |
| RHDV-V351 | 128 | 226 |
| RHDV-AST89 | 129 | 227 |
| LoV | 130 | 228 |
| NV | 131 | 229 |
| SHV | 132 | 230 |
| PYFV | 133 | 231 |
| RTSV-A | 134 | 232 |
| RTSV-V | 135 | 233 |
| AiV | 136 | 234 |
| HKV-1 | 137 | 235 |
| SV-A | 138 | 236 |
| HPeV-1 | 139 | 237 |
| HPeV-5 | 140 | 238 |
| HPeV-2 | 141 | 239 |
| LV | 142 | 240 |
| NoV | 143 | 241 |
| HAV-IA | 144 | 242 |
| HHAV | 145 | 243 |
| SHAV | 146 | 244 |
| AEV-CV | 147 | 245 |
| AEV-L | 148 | 246 |
| CosV | 149 | 247 |
| SVV | 150 | 248 |
| EMCV | 151 | 249 |
| MEMCV | 152 | 250 |
| SaFV | 153 | 251 |
| TMEV-B | 154 | 252 |
| TMEV-G | 155 | 253 |
| FMDV-S | 156 | 254 |
| FMDV-G | 157 | 255 |
| FMDV-A | 158 | 256 |
| HRV-1A | 159 | 257 |
| HRV-16 | 160 | 258 |
| HRV2 | 161 | 259 |
| HRV-89 | 162 | 260 |
| HRV-14 | 163 | 261 |
| HRV-3 | 164 | 262 |
| BEV | 165 | 263 |
| PEV-9 | 166 | 264 |
| EV-68 | 167 | 265 |
| EV-70 | 168 | 266 |
| EV-71 | 169 | 267 |
| Cox A16 | 170 | 268 |
| EV-71M | 171 | 269 |
| CV-A21 | 172 | 270 |
| CV-A24 | 173 | 271 |
| PV-1 | 174 | 272 |
| PV | 175 | 273 |
| E6-C | 176 | 274 |
| SVDV | 177 | 275 |
| CB5 | 178 | 276 |
| ECV12 | 179 | 277 |
| ECV11 | 180 | 278 |
| CAV-9 | 181 | 279 |
| CAV-B3 | 182 | 280 |
| CAV-B4 | 183 | 281 |
| E9 | 184 | 282 |
| E30 | 185 | 283 |

1. A Rep (e.g., Rep78, Rep68, Rep52, Rep40) protein comprising a helicase domain comprising an amino acid mutation relative to a reference helicase domain (e.g., SEQ ID NO: 186).

2. A polynucleotide encoding a Rep (e.g., Rep78, Rep68, Rep52, Rep40) protein comprising a helicase domain comprising an amino acid mutation relative to a reference helicase domain (e.g., SEQ ID NO: 186).

3. A polynucleotide encoding a Rep78 protein, a Rep68 protein, a Rep52 protein, and a Rep40 protein, wherein the Rep78 protein, the Rep68 protein, the Rep52 protein, and the Rep40 protein share a helicase domain comprising an amino acid mutation relative to a reference helicase domain (e.g., SEQ ID NO: 186).

4. A helicase comprising a helicase domain comprising an amino acid mutation relative to a reference helicase domain (e.g., SEQ ID NO: 186).

5. A polynucleotide encoding a helicase comprising a helicase domain comprising an amino acid mutation relative to a reference helicase domain (e.g., SEQ ID NO: 186).

6. A system for packaging an RNA into an AAV capsid to produce a recombinant RNA-packaged AAV particle (rRAAV particle),
wherein the RNA comprises:
(a) an RNA sequence of interest (RSI), e.g., an RNA sequence encoding a protein of interest, and
(b) an RNA-packaging signal (RPS) capable of interacting, e.g., binding, directly or indirectly, with an RPS-interacting molecule that facilitates packaging of the RNA into the AAV capsid;
wherein the system comprises:
(1) one or more capsid proteins (e.g., VP1, VP2, and/or VP3) for assembling the AAV capsid, or one or more coding sequences therefor, or a polynucleotide comprising said coding sequences;
(2) one or more Rep proteins (e.g., Rep78, Rep68, Rep52, Rep40) comprising a helicase domain comprising an amino acid mutation relative to a reference helicase domain (e.g., SEQ ID NO: 186), or one or more coding sequences therefor, or a polynucleotide comprising said coding sequences;
(3) the RPS-interacting molecule, or a coding sequence therefor, or a polynucleotide comprising said coding sequence;
(4) the RNA, or a coding sequence therefor, or a polynucleotide comprising said coding sequence, e.g., a transgene vector comprising or encoding the RNA; and
(5) optionally, one or more helper proteins required for AAV packaging (e.g., helper proteins from adenoviral E2a, E4, and/or VA genes), or one or more coding sequences therefor, or a polynucleotide comprising said coding sequences.

7. A method for the production of a recombinant RNA-packaged AAV particle (rRAAV particle), said method comprising:
a) culturing for a sufficient time a cell comprising a system for packaging an RNA into a AAV capsid to produce the recombinant RNA-packaged AAV particle (rRAAV particle), and
b) harvesting the rRAAV particle or a population thereof;
wherein the RNA comprises:
(a) an RNA sequence of interest (RSI), e.g., an RNA sequence encoding a protein of interest, and
(b) an RNA-packaging signal (RPS) capable of interacting, e.g., binding, directly or indirectly, with an RPS-interacting molecule that facilitates packaging of the RNA into the AAV capsid;
wherein the system comprises:
(1) one or more capsid proteins (e.g., VP1, VP2, and/or VP3) for assembling the AAV capsid, or one or more coding sequences therefor, or a polynucleotide comprising said coding sequences;
(2) one or more Rep proteins (e.g., Rep78, Rep68, Rep52, Rep40) comprising a helicase domain comprising an amino acid mutation relative to a reference helicase domain (e.g., SEQ ID NO: 186), or one or more coding sequences therefor, or a polynucleotide comprising said coding sequences;
(3) the RPS-interacting molecule, or a coding sequence therefor, or a polynucleotide comprising said coding sequence;
(4) the RNA, or a coding sequence therefor, or a polynucleotide comprising said coding sequence, e.g., a transgene vector comprising or encoding the RNA; and
(5) optionally, one or more helper proteins required for AAV packaging (e.g., helper proteins from adenoviral E2a, E4, and/or VA genes), or one or more coding sequences therefor, or a polynucleotide comprising said coding sequences.

8. Use of a Rep protein (e.g., Rep78, Rep68, Rep52, Rep40) comprising a helicase domain comprising an amino acid mutation relative to a reference helicase domain (e.g., SEQ ID NO: 186), or a polynucleotide encoding the Rep protein, in the production of a recombinant RNA-packaged AAV particle (rRAAV particle), said production comprising:
a) culturing for a sufficient time a cell comprising a system for packaging an RNA into a AAV capsid to produce the recombinant RNA-packaged AAV particle (rRAAV particle), and
b) harvesting the rRAAV particle or a population thereof;
wherein the RNA comprises:
(a) an RNA sequence of interest (RSI), e.g., an RNA sequence encoding a protein of interest, and
(b) an RNA-packaging signal (RPS) capable of interacting, e.g., binding, directly or indirectly, with an RPS-interacting molecule that facilitates packaging of the RNA into the AAV capsid;
wherein the system comprises:
(1) one or more capsid proteins (e.g., VP1, VP2, and/or VP3) for assembling the AAV capsid, or one or more coding sequences therefor, or a polynucleotide comprising said coding sequences;
(2) one or more said Rep proteins (e.g., Rep78, Rep68, Rep52, Rep40), or one or more coding sequences therefor, or a polynucleotide comprising said coding sequences;
(3) the RPS-interacting molecule, or a coding sequence therefor, or a polynucleotide comprising said coding sequence;
(4) the RNA, or a coding sequence therefor, or a polynucleotide comprising said coding sequence, e.g., a transgene vector comprising or encoding the RNA; and
(5) optionally, one or more helper proteins required for AAV packaging (e.g., helper proteins from adenoviral E2a, E4, and/or VA genes), or one or more coding sequences therefor, or a polynucleotide comprising said coding sequences.

9. The Rep protein, the helicase, the polynucleotide, the system, the method, or the use of any preceding claim, wherein the amino acid mutation leads to an increased RNA unwinding property and/or a decreased DNA unwinding property of the helicase or the Rep protein comprising the amino acid mutation.

10. The Rep protein, the helicase, the polynucleotide, the system, the method, or the use of any preceding claim, wherein the amino acid mutation leads to increased RNA packaging efficiency and/or decreased DNA packaging efficiency.

11. The Rep protein, the helicase, the polynucleotide, the system, the method, or the use of any preceding claim, wherein the reference helicase domain is the helicase domain of a reference helicase, e.g., a wild type helicase.

12. The Rep protein, the helicase, the polynucleotide, the system, the method, or the use of any preceding claim, wherein the reference helicase is a superfamily 3 (SF3) helicase.

13. The Rep protein, the helicase, the polynucleotide, the system, the method, or the use of any preceding claim, wherein the reference helicase is a helicase capable of unwinding DNA.

14. The Rep protein, the helicase, the polynucleotide, the system, the method, or the use of any preceding claim, wherein the reference helicase is a superfamily 3 (SF3) helicase capable of unwinding DNA.

15. The Rep protein, the helicase, the polynucleotide, the system, the method, or the use of any preceding claim, wherein the reference helicase domain is the helicase domain of a reference Rep protein, e.g., a wild type Rep protein.

16. The Rep protein, the helicase, the polynucleotide, the system, the method, or the use of any preceding claim, wherein the reference Rep protein is a reference Rep78 protein, a reference Rep68 protein, a reference Rep52 protein, or a reference Rep40 protein.

17. The Rep protein, the helicase, the polynucleotide, the system, the method, or the use of any preceding claim, wherein the reference helicase domain is the reference helicase domain shared by a reference Rep78 protein, a reference Rep68 protein, a reference Rep52 protein, and a reference Rep40 protein of a same AAV virus.

18. The Rep protein, the helicase, the polynucleotide, the system, the method, or the use of any preceding claim, wherein the reference Rep protein, the reference Rep78 protein, the reference Rep68 protein, the reference Rep52 protein, and the reference Rep40 protein are from a wild type AAV virus.

19. The Rep protein, the helicase, the polynucleotide, the system, the method, or the use of any preceding claim, wherein the wild type AAV virus has a serotype selected from the group consisting of AAV1, AAV2, AAV3A, AAV3B, AAV4, AAV5, AAV6, AAV7, AAV8, AAV9, AAV10, AAV11, AAV12, AAV13, AAVrh74, AAVrh10, AAV-DJ, AAV.PHP.eB, Anc80L65, Anc80L65AAP, and 7m8.

20. The Rep protein, the helicase, the polynucleotide, the system, the method, or the use of any preceding claim, wherein the reference helicase domain (e.g., SEQ ID NO: 186) comprises, from N- to C-terminus, Motif A, Motif B, Motif B′, Motif C, and Arginine Finger (R finger).

21. The Rep protein, the helicase, the polynucleotide, the system, the method, or the use of any preceding claim, wherein the reference Rep78 protein comprises, consists essentially of, or consists an amino acid sequence having a sequence identity of at least about 30% (e.g., at least about 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to the amino acid sequence of any one of SEQ ID NOs: 88-10⁹; wherein the reference Rep68 protein comprises, consists essentially of, or consists of the amino acid sequence of SEQ ID NO: 284 or a corresponding amino acid sequence comprised in any one of SEQ ID NOs: 89-10⁹; wherein the reference Rep52 protein comprises, consists essentially of, or consists of the amino acid sequence of SEQ ID NO: 285 or a corresponding amino acid sequence comprised in any one of SEQ ID NOs: 89-10⁹; or wherein the reference Rep40 protein comprises, consists essentially of, or consists of the amino acid sequence of SEQ ID NO: 286 or a corresponding amino acid sequence comprised in any one of SEQ ID NOs: 89-10⁹.

22. The Rep protein, the helicase, the polynucleotide, the system, the method, or the use of any preceding claim, wherein the reference helicase domain comprises, consists essentially of, or consists an amino acid sequence having a sequence identity of at least about 30% (e.g., at least about 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to the amino acid sequence of any one of SEQ ID NOs: 186-207.

23. The Rep protein, the helicase, the polynucleotide, the system, the method, or the use of any preceding claim, wherein the amino acid mutation is at a position corresponding to one or more positions of position 308 through position 463, optionally position 325 through position 461, of the amino acid sequence of any one of SEQ ID NOs: 186-207, wherein the position is numbered according to SEQ ID NO: 88.

24. The Rep protein, the helicase, the polynucleotide, the system, the method, or the use of any preceding claim, wherein the amino acid mutation is at a position corresponding to the position of a conserved amino acid (e.g., a position corresponding to A344 of SEQ ID NO: 88) across at least 80%, at least 90%, or 100% of the Rep proteins (e.g., SEQ ID NOs: 88-10⁹) of ssDNA viruses.

25. The Rep protein, the helicase, the polynucleotide, the system, the method, or the use of any preceding claim, wherein the amino acid mutation is at a position corresponding to a position in one or more of Motif A, Motif B, Motif B′, Motif C of the reference helicase domain, a upstream region no more than about 30, 25, 20, 15, 10, or 5 amino acids from the N-terminal of any one of Motif A, Motif B, Motif B′, Motif C, and Arginine Finger (R finger) of the reference helicase domain, and a downstream region no more than about 30, 25, 20, 15, 10, or 5 amino acids from the C-terminal of any one of Motif A, Motif B, Motif B′, Motif C, and Arginine Finger (R finger) of the reference helicase domain.

26. The Rep protein, the helicase, the polynucleotide, the system, the method, or the use of any preceding claim, wherein the Rep78 protein comprising said amino acid mutation comprises, consists essentially of, or consists an amino acid sequence having a sequence identity of at least about 30% (e.g., at least about 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9%) and less than 100% to the amino acid sequence of any one of SEQ ID NOs: 88-10⁹.

27. The Rep protein, the helicase, the polynucleotide, the system, the method, or the use of any preceding claim, wherein the helicase domain comprising said amino acid mutation comprises, consists essentially of, or consists an amino acid sequence having a sequence identity of at least about 30% (e.g., at least about 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9%) and less than 100% to the amino acid sequence of any one of SEQ ID NOs: 186-207.

28. The Rep protein, the helicase, the polynucleotide, the system, the method, or the use of any preceding claim, wherein the amino acid mutation comprises a mutation at a position corresponding to G325, R327, W331, A336, T337, I343, A344, D371, K372, M373, I375, E378, C405, T419, S420, T422, C425, Q442, D443, M445, K447, E449, L450, T451, L454, D455, H456, D457, F458, and/or V461 of the amino acid sequence of SEQ ID NO: 186, wherein the position is numbered according to SEQ ID NO: 88.

29. The Rep protein, the helicase, the polynucleotide, the system, the method, or the use of any preceding claim, wherein the amino acid mutation comprises a mutation at a position corresponding to A336, T337, I343, A344, K372, E378, D443, M445, K447, E449, L450, T451, L454, D455, H456, D457, F458, and/or V461 of the amino acid sequence of SEQ ID NO: 186, wherein the position is numbered according to SEQ ID NO: 88.

30. The Rep protein, the helicase, the polynucleotide, the system, the method, or the use of any preceding claim, wherein the amino acid mutation comprises a substitution corresponding to a substitution selected from the group consisting of G325P, G325I, K326E, K326R, R327P, N328V, W331I, F333H, F333Y, F333K, P335S, A336P, A336S, A336R, T337G, T338G, T341S, N342I, I343T, I343A, I343L, A344T, A344V, A344S, E345N, A346F, H349K, P352T, P365Y, N367D, D368G, C369Y, V370K, D371Q, D371G, D371N, K372Q, K372E, K372N, M373S, M373E, M373A, I375V, W376I, W377M, E378D, E379D, G380L, G380F, C405H, K406R, T419S, T419F, T419I, T419L, S420A, S420M, S420C, N421T, N421S, T422H, T422S, M424N, C425I, Q442K, Q442F, Q442H, Q442R, Q442L, Q442V, D443S, D443R, D443Y, D443N, D443A, M445I, M445R, M445F, F446R, F446I, K447F, K447N, K447H, K447P, K447T, K447A, E449D, E449I, E449R, L450M, L450I, L450V, T451D, T451I, T451E, T451K, T451N, L454F, L454V, D455F, D455K, D455H, D455Y, D455T, D455M, H456F, H456D, H456S, D457E, D457S, D457F, F458Y, F458K, V461L, and a combination thereof, wherein the position is numbered according to SEQ ID NO: 88.

31. The Rep protein, the helicase, the polynucleotide, the system, the method, or the use of any preceding claim, wherein the amino acid mutation comprises a substitution corresponding to a substitution selected from the group consisting of G325P, R327P, W331I, A336P, A336S, A336R, T337G, I343T, I343A, I343L, A344T, A344V, D371Q, K372Q, K372E, K372N, M373S, I375V, E378D, C405H, T419S, S420A, T422H, T422S, C425I, Q442H, Q442R, D443S, D443Y, D443N, D443A, M445I, K447F, K447N, K447T, E449D, L450M, L450I, L450V, T451D, T451E, L454F, D455F, D455Y, D455T, D455M, H456D, H456S, D457E, D457S, D457F, F458Y, V461L, and a combination thereof, wherein the position is numbered according to SEQ ID NO: 88.

32. The Rep protein, the helicase, the polynucleotide, the system, the method, or the use of any preceding claim, wherein the amino acid mutation comprises a substitution corresponding to a substitution selected from the group consisting of A336P, T337G, I343T, A344T, A344V, K372Q, E378D, D443S, M445I, K447F, E449D, L450M, T451D, L454F, D455F, D455T, H456D, D457E, F458Y, V461L, and a combination thereof, wherein the position is numbered according to SEQ ID NO: 88.

33. The Rep protein, the helicase, the polynucleotide, the system, the method, or the use of any preceding claim, wherein the amino acid mutation comprises or consists of a combination substitution corresponding a combination substitution selected from the group consisting of A336P+T337G, K372Q+E378D, D443S+M445I, D443S+L454F+D455F, K447F+E449D+T451D, K447F+L450M, K447F+F458Y, K447F+H456D+F458Y, K447F+V461L, E449D+L450M, L450M+T451D, L454F+D455F, D455T+H456D+D457E+F458Y, H456D+D457E+F458Y, F458Y+V461L, A344T+K372Q, A336P+A344T+K447F, A336P+A344V+K447F, I343T+K447F, I343T+L450M, A344T+K447F, A344V+K447F, A344T+L450M, A344V+L450M, A344T+K447F+E449D+T451D, K372Q+K447F, K372Q+L450M, K372Q++K447F+E449D+T451D, K372Q+V461L, E378D+K447F, E378D+L450M, E378D++K447F+E449D+T451D, A344T+K372Q+K447F, A344V+K372Q+K447F, and a combination thereof, wherein the position is numbered according to SEQ ID NO: 88.

34. The Rep protein, the helicase, the polynucleotide, the system, the method, or the use of any preceding claim, wherein the amino acid mutation comprises or consists of a combination substitution corresponding a combination substitution of A344V and K447F, wherein the position is numbered according to SEQ ID NO: 88.

35. The Rep protein, the helicase, the polynucleotide, the system, the method, or the use of any preceding claim, wherein the helicase domain comprising said amino acid mutation comprises, consists essentially of, or consists of the amino acid sequence of SEQ ID NO: 287.

36. The Rep protein, the helicase, the polynucleotide, the system, the method, or the use of any preceding claim, wherein the Rep protein comprising said amino acid mutation comprises, consists essentially of, or consists of the amino acid sequence of SEQ ID NO: 288.

37. The Rep protein, the helicase, the polynucleotide, the system, the method, or the use of any preceding claim, wherein the Rep protein comprise a mutation partially or substantially abolishing the endonuclease activity of the Rep protein, e.g., in the Original Binding Domain (OBD) of the Rep protein, optionally, the mutation comprises or consists of a mutation corresponding to a Y156F mutation, a K146A+D149A+E150A mutation (KDE-mu), or an E83A+K84A+E86A mutation (EKE-mu), wherein the position is numbered according to SEQ ID NO: 88.

38. The Rep protein, the helicase, the polynucleotide, the system, the method, or the use of any preceding claim, wherein Rep protein comprises a combination substitution comprising or consisting of Y156F, A344V, and K447F, wherein the position is numbered according to SEQ ID NO: 88.

39. The Rep protein, the helicase, the polynucleotide, the system, the method, or the use of any preceding claim, wherein the Rep protein comprise the amino acid sequence of SEQ ID NO: 289.

40. The Rep protein, the helicase, the polynucleotide, the system, the method, or the use of any preceding claim, wherein the RPS-interacting molecule is said Rep protein.

41. The Rep protein, the helicase, the polynucleotide, the system, the method, or the use of any preceding claim, wherein the RPS-interacting molecule comprises an RPS-binding protein (RPSBP) capable of binding directly or indirectly to the RNA packaging signal (RPS).

42. The Rep protein, the helicase, the polynucleotide, the system, the method, or the use of any preceding claim, wherein the Rep protein is (e.g., N-terminally, C-terminally, internally) fused with the RPSBP, optionally, via a peptide linker.

43. The Rep protein, the helicase, the polynucleotide, the system, the method, or the use of any preceding claim, wherein the RPS is located at or near the 5′ end of the RSI, at or near the 3′ end of the RSI, or internal to the RSI.

44. The Rep protein, the helicase, the polynucleotide, the system, the method, or the use of any preceding claim, wherein the RNA comprises one, two, or three copies of the RPS.

45. The Rep protein, the helicase, the polynucleotide, the system, the method, or the use of any preceding claim, wherein the RPS comprises an MS2 sequence (e.g., SEQ ID NO: 54), an PP7 binding site (e.g., SEQ ID NO: 56), and/or a Com binding site (e.g., SEQ ID NO: 58).

46. The Rep protein, the helicase, the polynucleotide, the system, the method, or the use of any preceding claim, wherein (a) the RPS comprises an MS2 sequence (e.g., SEQ ID NO: 54), and the RPSBP comprises a bacteriophage-derived MS2 coat protein (MCP) (e.g., SEQ ID NO: 49); (b) the RPS comprises an PP7 binding site (e.g., SEQ ID NO: 56), and the RPSBP comprises a PP7 bacteriophage coat protein (PCP) (e.g., SEQ ID NO: 51), or (c) the RPS comprises a Com binding site (e.g., SEQ ID NO: 58), and the RPSBP comprises a phage COM protein (COM) (e.g., SEQ ID NO: 53).

47. The Rep protein, the helicase, the polynucleotide, the system, the method, or the use of any preceding claim, wherein the RNA, or a coding sequence therefor, or a polynucleotide comprising said coding sequence, e.g., a transgene vector comprising or encoding the RNA, lacks a functional DNA packaging signal, e.g., an AAV ITR (such as, 5′ AAV2 ITR and/or 3′ AAV2 ITR), or a coding sequence therefor.

48. The Rep protein, the helicase, the polynucleotide, the system, the method, or the use of any preceding claim, wherein the RNA is transcribed from a polynucleotide (e.g., a transgene plasmid) lacking a functional DNA packaging signal, e.g., an AAV ITR (such as, 5′ AAV2 ITR and/or 3′ AAV2 ITR), or a coding sequence therefor.

49. The Rep protein, the helicase, the polynucleotide, the system, the method, or the use of any preceding claim, wherein the AAV capsid comprises a capsid from an AAV having a serotype selected from the group consisting of AAV1, AAV2, AAV3A, AAV3B, AAV4, AAV5, AAV6, AAV7, AAV8, AAV9, AAV10, AAV11, AAV12, AAV13, AAVrh74, AAVrh10, AAV-DJ, AAV.PHP.eB. Anc80L65, Anc80L65AAP, and 7m8.

50. The Rep protein, the helicase, the polynucleotide, the system, the method, or the use of any preceding claim, wherein the RNA is not bound to the AAV capsid.

51. A vector comprising the polynucleotide of any preceding claim; optionally, wherein the vector is a plasmid.

52. A (isolated) (host) cell comprising the Rep protein, the helicase, the polynucleotide, the system, or the vector of any preceding claim.

53. A recombinant RNA-packaged AAV particle (rRAAV particle) or a population thereof produced by the method of any preceding claim.
